(19) United States
(12) Patent Application Publication
(10) Pub. No.: US 2022/0144839 A1
(43) Pub. Date: May 12, 2022

(54) INDOLE AHR INHIBITORS AND USES THEREOF

(71) Applicant: Ikena Oncology, Inc., Boston, MA (US)

(72) Inventor: Alfredo C. CASTRO, Somerville, MA (US)

(21) Appl. No.: 17/281,395

(22) PCT Filed: Oct. 16, 2019
(86) PCT No.: PCT/US2019/056455

§ 371 Date: Mar. 30, 2021

Related U.S. Application Data
(60) Provisional application No. 62/746,277, filed on Oct. 16, 2018.

(51) Int. Cl. C07D 487/04; C07D 519/00; C07D 401/14; C07D 487/14

(52) U.S. Cl. CPC C07D 487/04; C07D 519/00; C07B 2200/05; C07D 487/14; C07D 401/14

(57) ABSTRACT

The present invention provides compounds useful as inhibitors of AHR, compositions thereof, and methods of using the same.

### BACKGROUND OF THE INVENTION

The aryl hydrocarbon receptor (AHR) is a transcription factor that without ligand exists in the inactive state in the cytoplasm bound to HSP90. Upon ligand binding, AHR translocates to the nucleus where it dimerizes with ARNT forming a functional transcription factor. AHR/ARNT binds dioxin response elements (DRE) in the promotor of many genes where it modulates gene transcription. The most well documented genes regulated by AHR are the cytochrome P450 genes Cyp1b1 and Cyp1a1, where activation of AHR greatly increases expression of these genes. Therefore, Cyp1b1 and Cyp1a1 mRNA levels are a selective readout of AHR activation (reviewed in Murray et al., 2014).

Many exogenous and endogenous agonists of AHR exist that activate the receptor. The best characterized exogenous ligand class are the dioxins. One of the first endogenous ligands to be characterized is kynurenine, generated by TDO (Opitz 2011) or IDO (Mezrich 2010). Kynurenine is a stable metabolite in the IDO/TDO pathway and is the product of tryptophan degradation. Kynurenine has been shown to activate AHR as measured by an increase in Cyp1a1 and/or Cyp1b1 mRNA levels in multiple cell types, along with other DRE-driven genes.

AHR activation has pro-tumor effects by acting directly on the tumor cells and indirectly by causing immunosuppression, therefore not allowing the body's own immune system to attack the tumor. For example, AHR activation through multiple ligands leads to increased expression of FoxP3 and results in a polarization of CD4+ T-cells toward a suppressive subset called Foxp3+ T-regulatory cells (Tregs). These T-reg cells inhibit the proliferation of activated T cells (Funatake 2005, other refs). Interestingly, kynurenine has been shown to induce immunosuppressive Tregs through AHR. Kynurenine does not affect T-reg generation in AHR-null T cells or when an AHR antagonist is added (Mezrich). In addition to T-regs, AHR activation also leads to expansion of suppressive Tr1 T cells (Gandhi 2010). It has also been shown that expression of IDO is regulated by AHR activation in both tumor cells and T cells, leading to increased immune suppression (Vogel). It is likely there is also a role for AHR in immune suppressive myeloid cells (Nguyen 2013). Immune suppression is often associated with high levels of anti-inflammatory cytokines and there is evidence that AHR is involved in activation of many of these cytokines, such as IL-10 (Gandhi 2010, Wagage 2014).

There remains an unmet need to develop inhibitors of AHR for treating diseases, disorders and conditions associated therewith.

### SUMMARY OF THE INVENTION

It has now been found that compounds of this invention, and pharmaceutically acceptable compositions thereof, are effective as inhibitors of AHR. Such compounds have the general formula I:

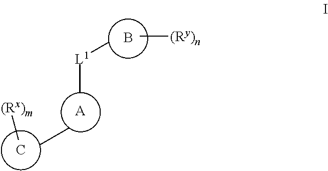

or a pharmaceutically acceptable salt thereof, wherein each variable is as defined and described herein.

Compounds of the present invention, and pharmaceutically acceptable compositions thereof, are useful for treating a variety of diseases, disorders or conditions, associated with AHR. Such diseases, disorders, or conditions include those described herein.

Compounds provided by this invention are also useful for the study of AHR in biological and pathological phenomena; the study of intracellular signal transduction pathways; and the comparative evaluation of new AHR inhibitors in vitro or in vivo.

### DETAILED DESCRIPTION OF CERTAIN EMBODIMENTS

### 1. General Description of Compounds of the Invention

In certain embodiments, the present invention provides inhibitors of AHR. In some embodiments, such compounds include those of formula I.

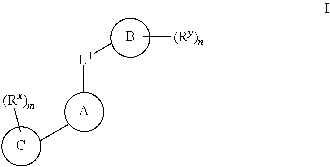

or a pharmaceutically acceptable salt thereof, wherein:
Ring A is selected from:

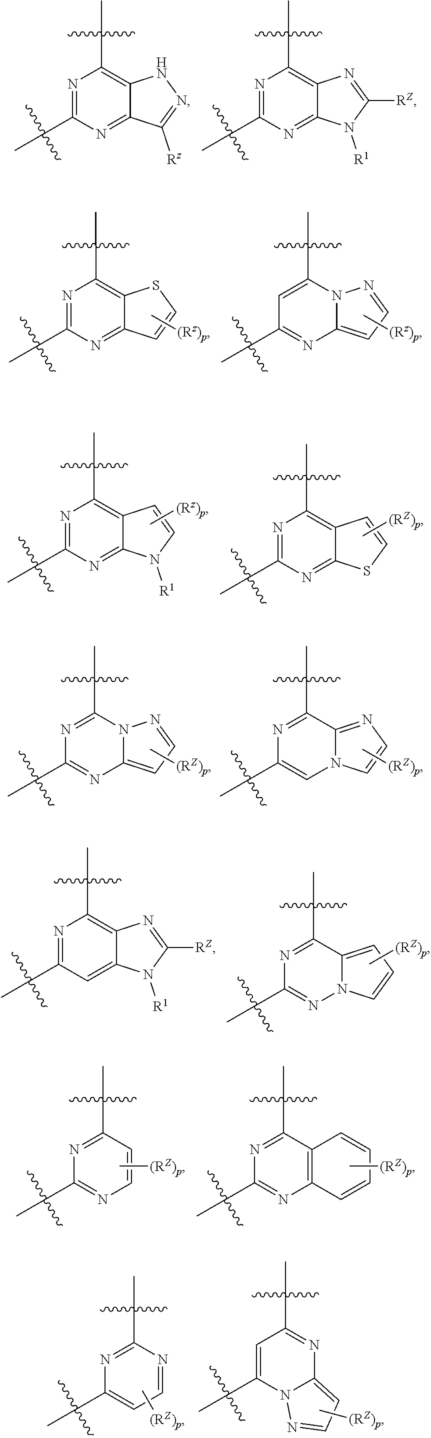

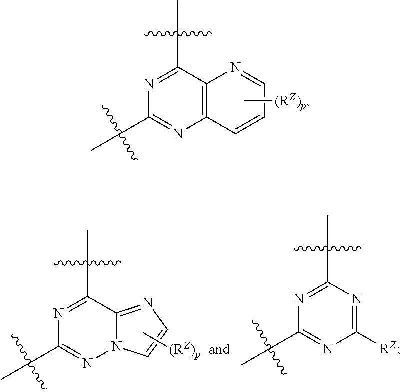

each p is independently 0, 1, or 2, as valency will allow;
each R¹ is independently selected from R, —C(O)R, —C(O)OR, —SO₂R, —C(O)N(R)₂, or —SO₂RN(R)₂;
each R is independently hydrogen, deuterium, or an optionally substituted group selected from C₁₋₆ aliphatic, a 3-8 membered saturated or partially unsaturated monocyclic carbocyclic ring, phenyl, an 8-10 membered bicyclic aromatic carbocyclic ring; a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur; or two R on the same nitrogen are taken together with their intervening atoms to form a 4-7 membered saturated, partially unsaturated, or aromatic ring having 1-2 heteroatoms in addition to the nitrogen independently selected from oxygen, nitrogen, or sulfur;
each or R^{x}, R^{y}, and R^{z} is independently selected from R, halogen, cyano, nitro, —OR, —SR, —N(R)₂, —N(R)C(O)R, —C(O)N(R)₂, —C(O)N(R)OR, —N(R)C(O)N(R)₂, —N(R)C(O)OR, —OC(O)N(R)₂, —N(R)SO₂R, —SO₂RN(R)₂, —C(O)R, —C(O)OR, —OC(O)R, —C(O)OR, —S(O)R, or —SO₂R, or:
two R^{z} on adjacent atoms are taken together with their intervening atoms to form a 3-7 membered saturated, partially unsaturated, or aromatic ring having 0-2 heteroatoms independently selected from nitrogen, oxygen, or sulfur;
two R^{x} on the same carbon are taken together to form ═O or ═S; or:
two R^{y} on the same carbon are taken together to form ═O or ═S;
each of m and n is independently 1, 2, 3, 4, or 5;
Ring B is absent or a 4-8 membered saturated or partially unsaturated carbocyclic ring; phenyl, a 7-10 membered bicyclic partially unsaturated or aromatic carbocyclic ring, a 5-6 membered monocyclic heteroaromatic ring having 1-4 heteroatoms independently selected from nitrogen, oxygen, or sulfur, an 8-10 membered bicyclic partially unsaturated or heteroaryl ring having 1-4 heteroatoms independently selected from nitrogen, oxygen, or sulfur, or a 12-15 membered partially unsaturated or aromatic tricyclic ring having 1-4 heteroatoms independently selected from nitrogen, oxygen, or sulfur;
Ring C is phenyl, a 5-6 membered saturated, partially unsaturated or heteroaryl ring having 1-4 heteroatoms independently selected from nitrogen, oxygen, or sulfur, or an 8-10 membered bicyclic partially unsaturated or heteroaryl ring having 1-4 heteroatoms independently selected from nitrogen, oxygen, or sulfur;
L¹ is a covalent bond or an optionally substituted C₁₋₆ membered straight or branched bivalent hydrocarbon chain wherein a methylene unit of L¹ is optionally replaced with -Cy-, —O—, —S—, —NR—, —C(O)—, —C(O)O—, —OC(O)—, —C(O)N(R)—, —N(R)C(O)—, —SO₂—, —N(R)SO₂—, or —SO₂N(R)—S; and
-Cy- is a 3-8 membered bivalent saturated, partially unsaturated, or aromatic monocyclic ring having 0-2 heteroatoms independently selected from nitrogen, oxygen, or sulfur, or an 8-10 membered bivalent saturated, partially unsaturated, or aromatic bicyclic ring having 0-3 heteroatoms independently selected from nitrogen, oxygen, or sulfur.

In some embodiments, the present invention provides a compound of formula I, with the proviso that when Ring A is

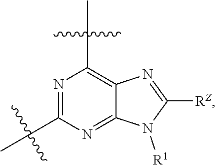

### Ring B is not

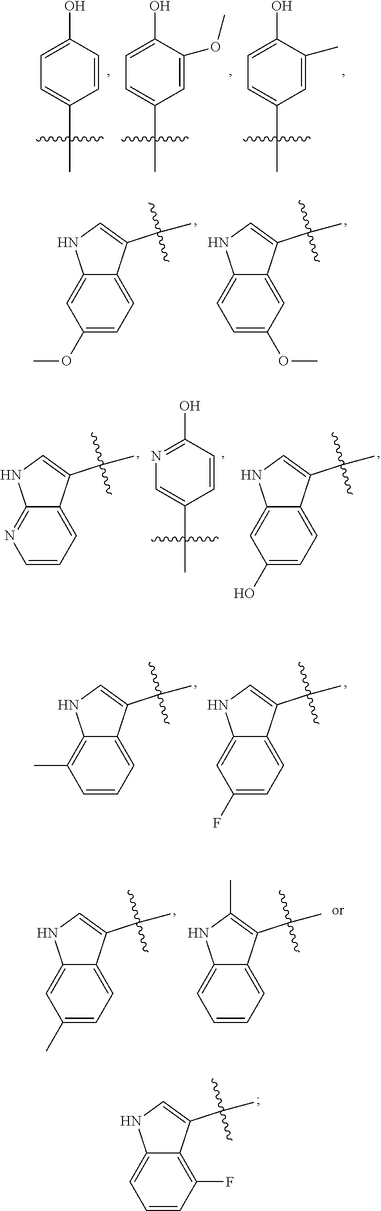

and/or Ring C is not

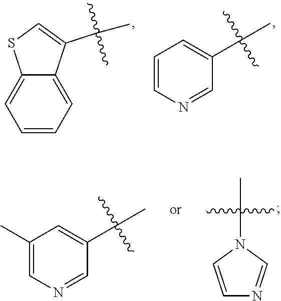

and/or R¹ is not

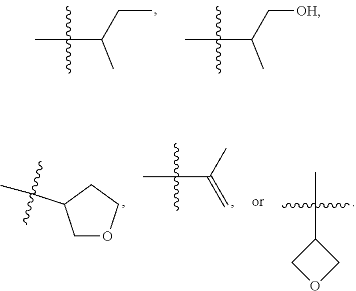

As generally defined above, Ring A is selected from:

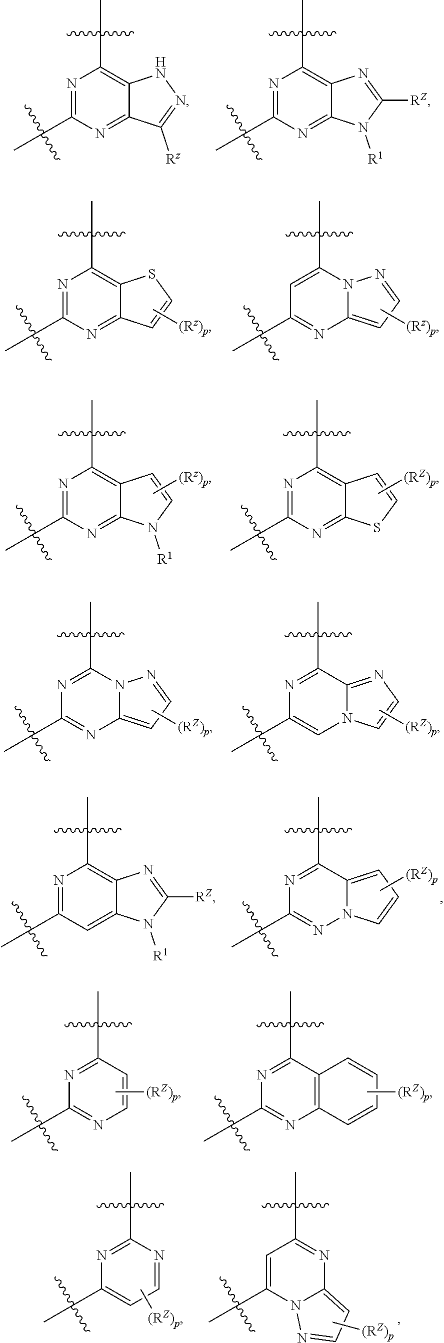

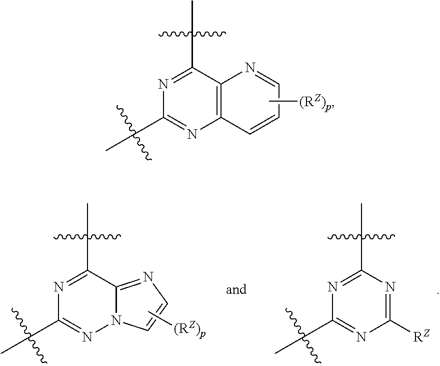

One of ordinary skill in the art would readily understand and appreciate that there are multiple orientations of Ring A. For example, and for the purposes of clarity, when Ring A is selected to be

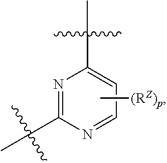

embodiments may be envisioned whereby Ring A is oriented in formula I as

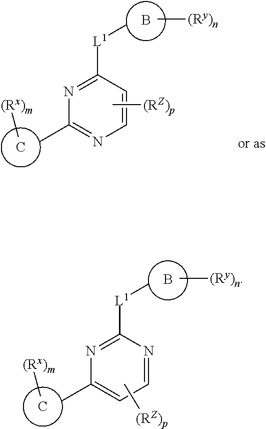

Accordingly, both such orientations are contemplated by the present invention.

In some embodiments, the present invention provides a compound of formula I, with the proviso that L¹ is not —NHCH₂CH₂—. In some embodiments, the present invention provides a compound of formula I, with the proviso that when Ring A is

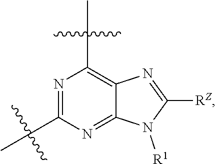

L¹ is not —NHCH₂CH₂—.

### 2. Compounds and Definitions

Compounds of this invention include those described generally above, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

As used herein, the term “cyclopropylenyl” refers to a bivalent cyclopropyl group of the following structure:

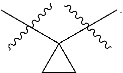 [SMILES: *C1(*)CC1]

As used herein, the term “cyclobutylenyl” refers to a bivalent cyclobutyl group of the following structure:

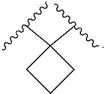 [SMILES: *C1(*)CCC1]

As used herein, the term “oxetanyl” refers to a bivalent oxetanyl group of the following structure:

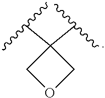 [SMILES: *C1(*)COC1]

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.”

The term “aryl” used alone or as part of a larger moiety as in “aralkyl,” “aralkoxy,” or “aryloxyalkyl,” refers to monocyclic and bicyclic ring systems having a total of five to 10 ring members, wherein at least one ring in the system is aromatic and wherein each ring in the system contains three to seven ring members. The term “aryl” may be used interchangeably with the term “aryl ring”. In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Non-limiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O) R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄ C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}; —(CH₂)₀₋₄ S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂— (5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR_{●}, —(CH₂)₀₋₂CH(OR^{●})₂; —O(haloR^{●}), —CN, —N₃, —(CH₂)₀₋₂C(O)R^{●}, —(CH₂)₀₋₂OC(O)OH, —(CH₂)₀₋₂OC(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R^{●} include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

### 3. Description of Exemplary Embodiments

In certain embodiments, the present invention provides inhibitors of AHR. In some embodiments, such compounds include those of formula I:

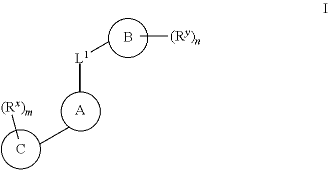

or a pharmaceutically acceptable salt thereof, wherein:
Ring A is selected from:

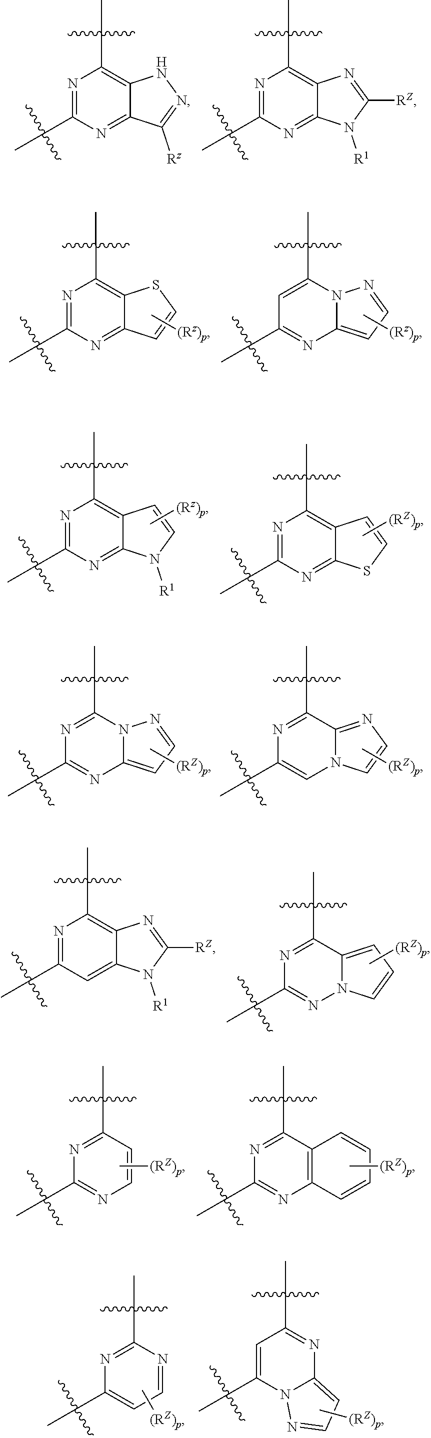

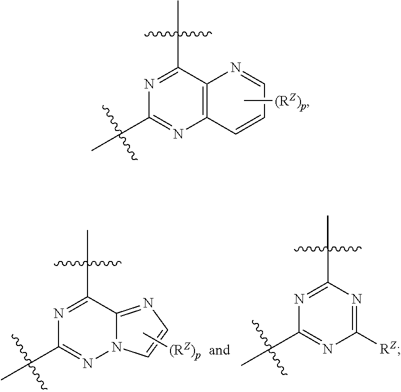

each p is independently 0, 1, or 2, as valency will allow;
each R¹ is independently selected from R, —C(O)R, —C(O)OR, —SO₂R, —C(O)N(R)₂, or —SO₂RN(R)₂;
each R is independently hydrogen, deuterium, or an optionally substituted group selected from C₁₋₆ aliphatic, a 3-8 membered saturated or partially unsaturated monocyclic carbocyclic ring, phenyl, an 8-10 membered bicyclic aromatic carbocyclic ring; a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur; or two R on the same nitrogen are taken together with their intervening atoms to form a 4-7 membered saturated, partially unsaturated, or aromatic ring having 1-2 heteroatoms in addition to the nitrogen independently selected from oxygen, nitrogen, or sulfur;
each or R^{x}, R^{y}, and R^{z} is independently selected from R, halogen, cyano, nitro, —OR, —SR, —N(R)₂, —N(R)C(O)R, —C(O)N(R)₂, —C(O)N(R)OR, —N(R)C(O)N(R)₂, —N(R)C(O)OR, —OC(O)N(R)₂, —N(R)SO₂R, —SO₂RN(R)₂, —C(O)R, —C(O)OR, —OC(O)R, —C(O)OR, —S(O)R, or —SO₂R, or:
   two R^{z} on adjacent atoms are taken together with their intervening atoms to form a 3-7 membered saturated, partially unsaturated, or aromatic ring having 0-2 heteroatoms independently selected from nitrogen, oxygen, or sulfur;
   two R^{x} on the same carbon are taken together to form ═O or ═S; or:
   two R^{y} on the same carbon are taken together to form ═O or ═S;
each of m and n is independently 1, 2, 3, 4, or 5;
Ring B is absent or a 4-8 membered saturated or partially unsaturated carbocyclic ring; phenyl, a 7-10 membered bicyclic partially unsaturated or aromatic carbocyclic ring, a 5-6 membered monocyclic heteroaromatic ring having 1-4 heteroatoms independently selected from nitrogen, oxygen, or sulfur, an 8-10 membered bicyclic partially unsaturated or heteroaryl ring having 1-4 heteroatoms independently selected from nitrogen, oxygen, or sulfur, or a 12-15 membered partially unsaturated or aromatic tricyclic ring having 1-4 heteroatoms independently selected from nitrogen, oxygen, or sulfur;
Ring C is phenyl, a 5-6 membered saturated, partially unsaturated or heteroaryl ring having 1-4 heteroatoms independently selected from nitrogen, oxygen, or sulfur, or an 8-10 membered bicyclic partially unsaturated or heteroaryl ring having 1-4 heteroatoms independently selected from nitrogen, oxygen, or sulfur;
L¹ is a covalent bond or an optionally substituted C¹⁻⁶ membered straight or branched bivalent hydrocarbon chain wherein a methylene unit of L¹ is optionally replaced with -Cy-, —O—, —S—, —NR—, —C(O)—, —C(O)O—, —OC(O)—, —C(O)N(R)—, —N(R)C(O)—, —SO₂—, —N(R)SO₂—, or —SO₂N(R)—S; and
-Cy- is a 3-8 membered bivalent saturated, partially unsaturated, or aromatic monocyclic ring having 0-2 heteroatoms independently selected from nitrogen, oxygen, or sulfur, or an 8-10 membered bivalent saturated, partially unsaturated, or aromatic bicyclic ring having 0-3 heteroatoms independently selected from nitrogen, oxygen, or sulfur.

In some embodiments, the present invention provides a compound of formula I, with the proviso that when Ring A is

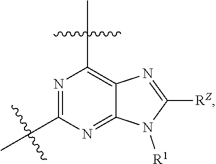

### Ring B is not

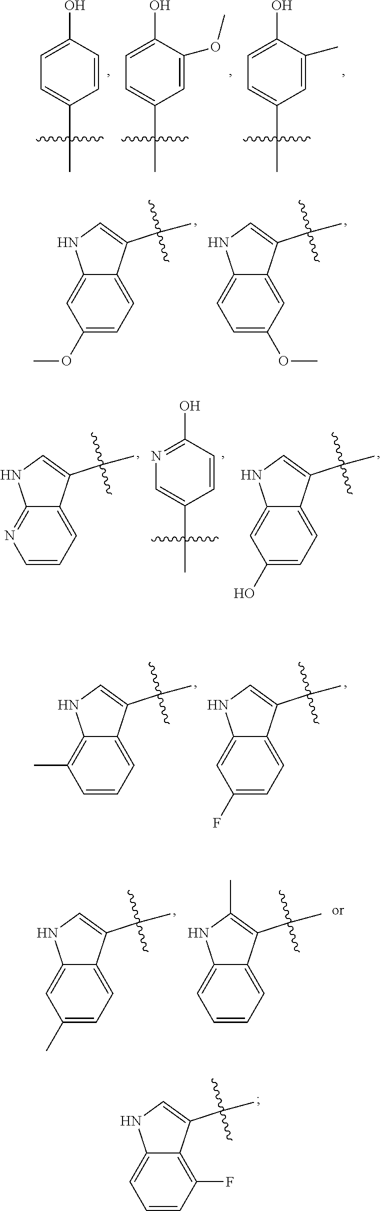

and/or Ring C is not

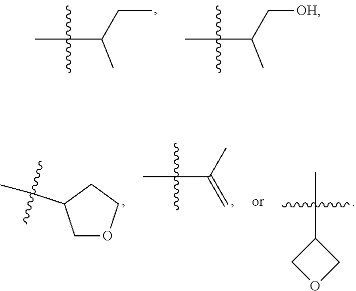

and/or R¹ is not

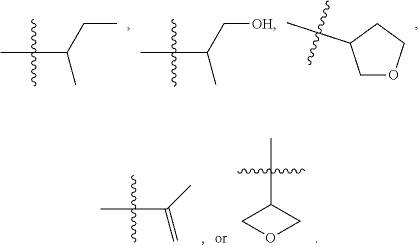

In some embodiments, the present invention provides a compound of formula I, with the proviso that L¹ is not —NHCH₂CH₂—. In some embodiments, the present invention provides a compound of formula I, with the proviso that when Ring A is

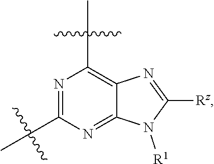

L¹ is not —NHCH₂CH₂—.

In some embodiments, the present invention provides a compound of formula I, with the proviso that the compound is other than:

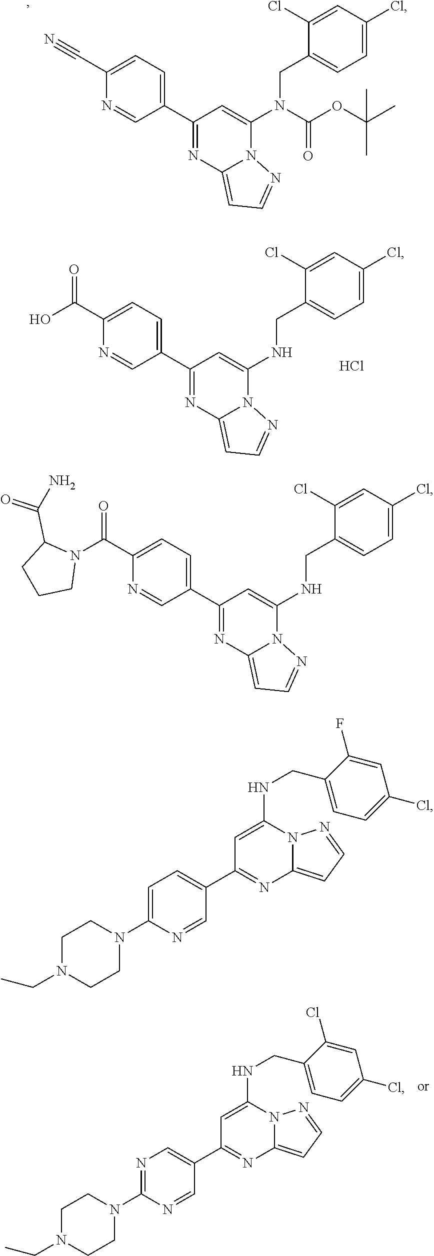

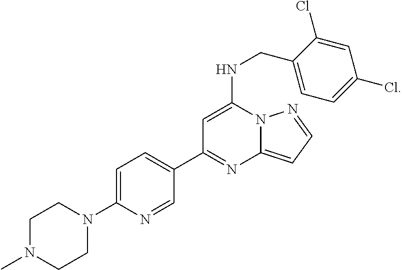 [SMILES: CN1CCN(c2ccc(-c3cc(NCc4ccc(Cl)cc4Cl)n4nccc4n3)cn2)CC1]

In some embodiments, a provided compound is other than

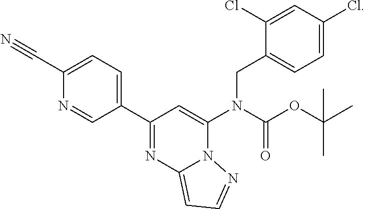 [SMILES: CC(C)(C)OC(=O)N(Cc1ccc(Cl)cc1Cl)c1cc(-c2ccc(C#N)nc2)nc2ccnn12]

In some embodiments, a provided compound is other than

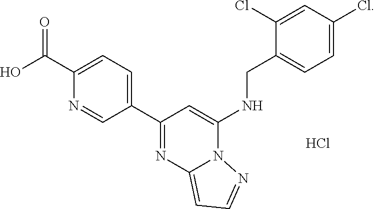 [SMILES: O=C(O)c1ccc(-c2cc(NCc3ccc(Cl)cc3Cl)n3nccc3n2)cn1 ; Cl]

In some embodiments, a provided compound is other than

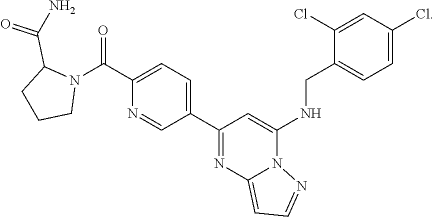 [SMILES: NC(=O)C1CCCN1C(=O)c1ccc(-c2cc(NCc3ccc(Cl)cc3Cl)n3nccc3n2)cn1]

In some embodiments, a provided compound is other than

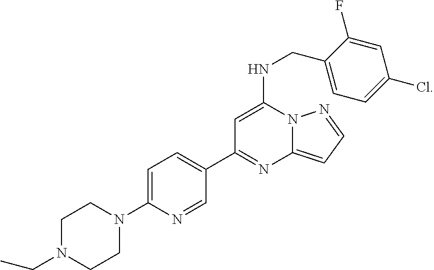 [SMILES: CCN1CCN(c2ccc(-c3cc(NCc4ccc(Cl)cc4F)n4nccc4n3)cn2)CC1]

In some embodiments, a provided compound is other than

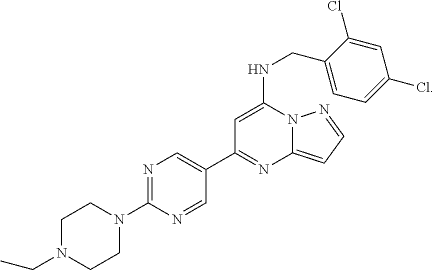 [SMILES: CCN1CCN(c2ncc(-c3cc(NCc4ccc(Cl)cc4Cl)n4nccc4n3)cn2)CC1]

In some embodiments, a provided compound is other than

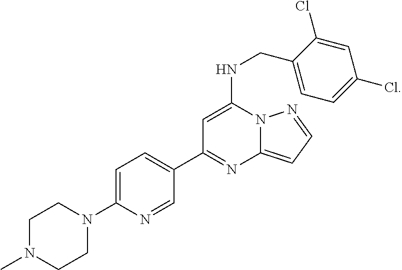 [SMILES: CN1CCN(c2ccc(-c3cc(NCc4ccc(Cl)cc4Cl)n4nccc4n3)cn2)CC1]

As defined generally above, R¹ is R, —C(O)R, —C(O)OR, —SO₂R, —C(O)N(R)₂, or —SO₂RN(R)₂. In some embodiments, R¹ is hydrogen. In some embodiments, R¹ is R. In some embodiments, R¹ is —C(O)R. In some embodiments, R¹ is —C(O)OR. In some embodiments, R¹ is —SO₂R. In some embodiments, R¹ is —C(O)N(R)₂. In some embodiments, R¹ is —SO₂RN(R)₂. In some embodiments, R¹ is hydrogen. In some embodiments, R¹ is deuterium. In some embodiments, R¹ is an optionally substituted group selected from C₁₋₆ aliphatic. In some embodiments, R¹ is selected from those depicted in Table 1, below.

As defined generally above, each R^{x} is independently R, halogen, cyano, nitro, —OR, —SR, —N(R)₂, —N(R)C(O)R, —C(O)N(R)₂, —C(O)N(R)OR, —N(R)C(O)N(R)₂, —N(R)C(O)OR, —OC(O)N(R)₂, —N(R)SO₂R, —SO₂RN(R)₂, —C(O)R, —C(O)OR, —OC(O)R, —C(O)OR, —S(O)R, or —SO₂R, or two R^{x} on the same carbon are taken together to form ═O or ═S. In some embodiments, each R^{x} is the same. In some embodiments, each R^{x} is different. In some embodiments, R^{x} is hydrogen. In some embodiments, R^{x} is R. In some embodiments, R^{x} is halogen. In some embodiments, R^{x} is cyano. In some embodiments, R^{x} is nitro. In some embodiments, R^{x} is —OR. In some embodiments, R^{x} is —SR. In some embodiments, R^{x} is —N(R)₂. In some embodiments, R^{x} is —N(R)C(O)R. In some embodiments, R^{x} is —C(O)N(R)₂. In some embodiments, R^{x} is —C(O)N(R)OR. In some embodiments, R^{x} is —N(R)C(O)N(R)₂. In some embodiments, R^{x} is —N(R)C(O)OR. In some embodiments, R^{x} is —OC(O)N(R)₂. In some embodiments, R^{x} is —N(R)SO₂R. In some embodiments, R^{x} is —SO₂RN(R)₂. In some embodiments, R^{x} is —C(O)R. In some embodiments, R^{x} is —C(O)OR. In some embodiments, R^{x} is —CO(O)R. In some embodiments, R^{x} is —S(O)R. In some embodiments, R^{x} is —SO₂R. In some embodiments, two R^{x} on the same carbon are taken together to form ═O or ═S. In some embodiments, R^{x} is hydrogen. In some embodiments, R^{x} is deuterium. In some embodiments, R^{x} is an optionally substituted group selected from C₁₋₆ aliphatic. In some embodiments, R^{x} is selected from those depicted in Table 1, below.

As defined generally above, each R^{y} is independently R, halogen, cyano, nitro, —OR, —SR, —N(R)₂, —N(R)C(O)R, —C(O)N(R)₂, —C(O)N(R)OR, —N(R)C(O)N(R)₂, —N(R)C(O)OR, —OC(O)N(R)₂, —N(R)SO₂R, —SO₂RN(R)₂, —C(O)R, —C(O)OR, —OC(O)R, —C(O)OR, —S(O)R, or —SO₂R, or two R^{y} on the same carbon are taken together to form ═O or ═S. In some embodiments, each R^{y} is the same. In some embodiments, each R^{y} is different. In some embodiments, R^{y} is hydrogen. In some embodiments, R^{y} is R. In some embodiments, R^{y} is halogen. In some embodiments, R^{y} is cyano. In some embodiments, R^{y} is nitro. In some embodiments, R^{y} is —OR. In some embodiments, R^{y} is —SR. In some embodiments, R^{y} is —N(R)₂. In some embodiments, R^{y} is —C(O)N(R)OR. In some embodiments, R^{y} is —N(R)C(O)R. In some embodiments, R^{y} is —C(O)N(R)₂. In some embodiments, R^{y} is —N(R)C(O)N(R)₂. In some embodiments, R^{y} is —N(R)C(O)OR. In some embodiments, R^{y} is —OC(O)N(R)₂. In some embodiments, R^{y} is —N(R)SO₂R. In some embodiments, R^{y} is —SO₂RN(R)₂. In some embodiments, R^{y} is —C(O)R. In some embodiments, R^{y} is —C(O)OR. In some embodiments, R^{y} is —CO(O)R. In some embodiments, R^{y} is —S(O)R. In some embodiments, R^{y} is —SO₂R. In some embodiments, two R^{y} on the same carbon are taken together to form ═O or ═S. In some embodiments, R^{y} is hydrogen. In some embodiments, R^{y} is deuterium. In some embodiments, R^{y} is an optionally substituted group selected from C₁₋₆ aliphatic. In some embodiments, R^{y} is selected from those depicted in Table 1, below.

As defined generally above, each R^{z} is independently R, halogen, cyano, nitro, —OR, —SR, —N(R)₂, —N(R)C(O)R, —C(O)N(R)₂, —C(O)N(R)OR, —N(R)C(O)N(R)₂, —N(R)C(O)OR, —OC(O)N(R)₂, —N(R)SO₂R, —SO₂RN(R)₂, —C(O)R, —C(O)OR, —OC(O)R, —C(O)OR, —S(O)R, or —SO₂R; or two R^{z} on adjacent atoms are taken together with their intervening atoms to form a 3-7 membered saturated, partially unsaturated, or aromatic ring having 0-2 heteroatoms independently selected from nitrogen, oxygen, or sulfur. In some embodiments, R^{z} is hydrogen. In some embodiments, R^{z} is R. In some embodiments, R^{z} is halogen. In some embodiments, R^{z} is cyano. In some embodiments, R^{z} is nitro. In some embodiments, R^{z} is —OR. In some embodiments, R^{z} is —SR. In some embodiments, R^{z} is —N(R)₂. In some embodiments, R^{z} is —C(O)N(R)OR. In some embodiments, R^{z} is —N(R)C(O)R. In some embodiments, R^{z} is —C(O)N(R)₂. In some embodiments, R^{z} is —N(R)C(O)N(R)₂. In some embodiments, R^{z} is —N(R)C(O)OR. In some embodiments, R^{z} is —OC(O)N(R)₂. In some embodiments, R^{z} is —N(R)SO₂R. In some embodiments, R^{z} is —SO₂RN(R)₂. In some embodiments, R^{z} is —C(O)R. In some embodiments, R^{z} is —C(O)OR. In some embodiments, R^{z} is —CO(O)R. In some embodiments, R^{z} is —S(O)R. In some embodiments, R^{z} is —SO₂R. In some embodiments, R^{z} is hydrogen. In some embodiments, R^{z} is deuterium. In some embodiments, R^{z} is an optionally substituted group selected from C₁₋₆ aliphatic. In some embodiments, two Ron adjacent atoms are taken together with their intervening atoms to form a 3-7 membered saturated, partially unsaturated, or aromatic ring having 0-2 heteroatoms independently selected from nitrogen, oxygen, or sulfur. In some embodiments, R^{z} is selected from those depicted in Table 1, below.

As defined generally above, p is 0, 1 or 2. In some embodiments, p is 0. In some embodiments, p is 1. In some embodiments, p is 2. In some embodiments, p is selected from those depicted in Table 1, below.

As defined generally above, n is 1, 2, 3, 4 or 5. In some embodiments, n is 1. In some embodiments, n is 2. In some embodiments, n is 3. In some embodiments, n is 4. In some embodiments, n is 5. In some embodiments, n is selected from those depicted in Table 1, below.

As defined generally above, m is 1, 2, 3, 4 or 5. In some embodiments, m is 1. In some embodiments, m is 2. In some embodiments, m is 3. In some embodiments, m is 4. In some embodiments, m is 5. In some embodiments, m is selected from those depicted in Table 1, below.

As defined generally above, Ring B is absent or a 4-8 membered saturated or partially unsaturated carbocyclic ring; phenyl, a 7-10 membered bicyclic partially unsaturated or aromatic carbocyclic ring, a 5-6 membered monocyclic heteroaromatic ring having 1-4 heteroatoms independently selected from nitrogen, oxygen, or sulfur, an 8-10 membered bicyclic partially unsaturated or heteroaryl ring having 1-4 heteroatoms independently selected from nitrogen, oxygen, or sulfur, or a 12-15 membered partially unsaturated or aromatic tricyclic ring having 1-4 heteroatoms independently selected from nitrogen, oxygen, or sulfur. In some embodiments, Ring B is absent. In some embodiments, Ring B is a 4-8 membered saturated or partially unsaturated carbocyclic ring. In some embodiments, Ring B is a 7-10 membered bicyclic partially unsaturated or aromatic carbocyclic ring. In some embodiments, Ring B is a 12-15 membered partially unsaturated or aromatic tricyclic ring having 1-4 heteroatoms independently selected from nitrogen, oxygen, or sulfur. In some embodiments, Ring B is a 5-6 membered monocyclic heteroaromatic ring having 1-4 heteroatoms independently selected from nitrogen, oxygen, or sulfur. In some embodiments, Ring B is an 8-10 membered bicyclic partially unsaturated or heteroaryl ring having 1-4 heteroatoms independently selected from nitrogen, oxygen, or sulfur. In some embodiments, Ring B is phenyl. In some embodiments, Ring B is thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl or pteridinyl, indolycarl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, tetrahydroquinolinyl, tetrahydroisoquinolinyl, or pyrido[2,3-b]-1,4-oxazin-3(4H)-one. In some embodiments, Ring B is selected from those depicted in Table 1, below.

As defined generally above, Ring C is phenyl, a 5-6 membered saturated, partially unsaturated or heteroaryl ring having 1-4 heteroatoms independently selected from nitrogen, oxygen, or sulfur, or an 8-10 membered bicyclic partially unsaturated or heteroaryl ring having 1-4 heteroatoms independently selected from nitrogen, oxygen, or sulfur. In some embodiments, Ring C is phenyl. In some embodiments, Ring C is a 5-6 membered saturated, partially unsaturated or heteroaryl ring having 1-4 heteroatoms independently selected from nitrogen, oxygen, or sulfur. In some embodiments, Ring C is an 8-10 membered bicyclic partially unsaturated or heteroaryl ring having 1-4 heteroatoms independently selected from nitrogen, oxygen, or sulfur. In some embodiments, Ring C is thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl or pteridinyl, indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, tetrahydroquinolinyl, tetrahydroisoquinolinyl, or pyrido[2,3-b]-1,4-oxazin-3(4H)-one. In some embodiments, Ring C is selected from those depicted in Table 1, below.

As defined generally above, L¹ is a covalent bond or an optionally substituted C₁₋₆ membered straight or branched bivalent hydrocarbon chain wherein a methylene unit of L¹ is optionally replaced with -Cy-, —O—, —S—, —NR—, —C(O)—, —C(O)O—, —OC(O)—, —C(O)N(R)—, —N(R)C(O)—, —SO₂—, —N(R)SO₂—, or —SO₂N(R)—S. In some embodiments, L¹ is a covalent bond. In some embodiments, L¹ is an optionally substituted C₁₋₆ membered straight or branched bivalent hydrocarbon chain. In some embodiments, L¹ is -Cy-. In some embodiments, L¹ is phenylene, heterocyclylene, heteroarylene, cyclopropylene, cyclobutylenyl, cyclopentylene, cyclohexylene or oxetanyl. In some embodiments, L¹ is —NR—. In some embodiments, L¹ is —N(CH₂)₂—. In some embodiments, L¹ is selected from those depicted in Table 1, below.

In some embodiments, -Cy- is phenylene, heterocyclylene, heteroarylene, cyclopropylene, cyclobutylenyl, cyclopentylene, cyclohexylene and oxetanyl. In some embodiments, -Cy- is selected from:

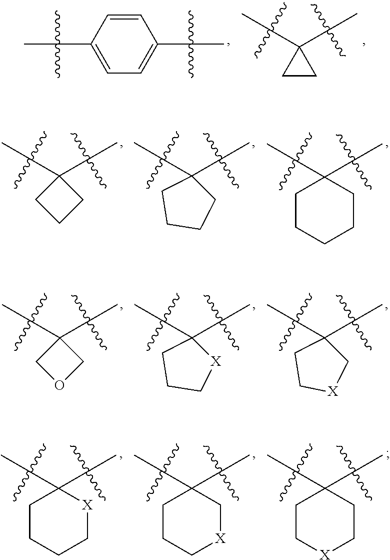

wherein X is a heteroatom selected from nitrogen, oxygen, or sulfur. In some embodiments, -Cy- is selected from those depicted in Table 1, below.

In some embodiments, the present invention provides a compound selected from any of formulae I-a, I-b, I-c, I-d, I-e, I-f, I-g, I-h, I-i, I-j, I-k, I-l, I-m, I-n, I-o, I-p, I-q, I-r, I-s, I-t and I-u:

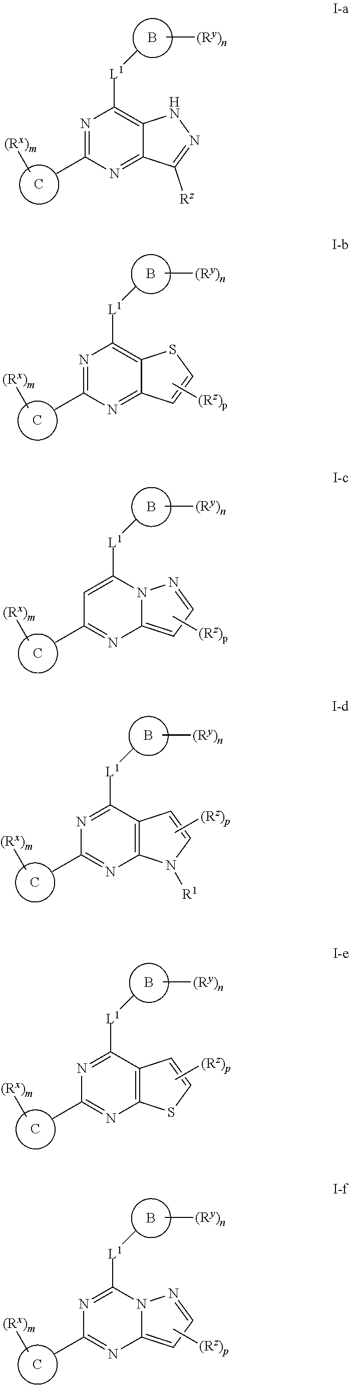

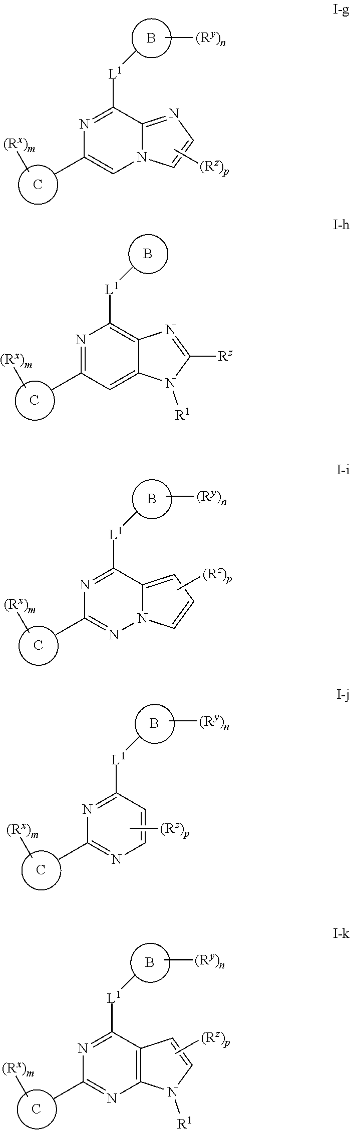

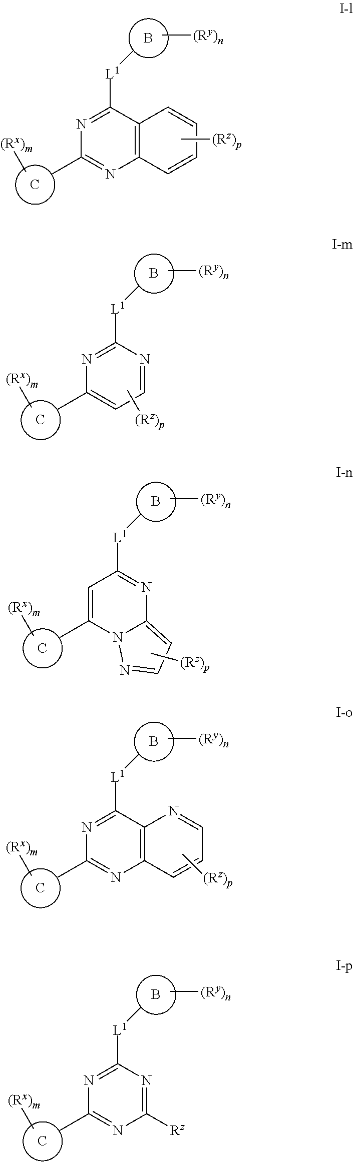

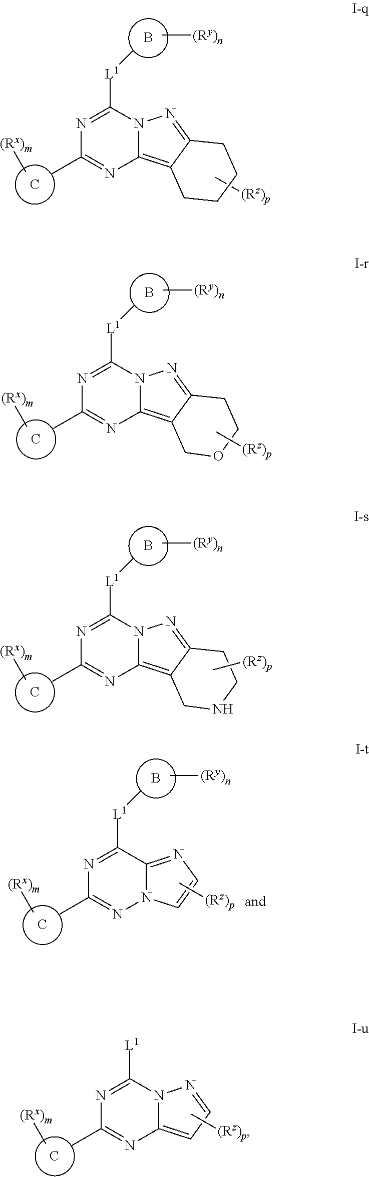

pharmaceutically acceptable salt thereof; wherein each variable is as defined herein and described in embodiments for formula I, supra, or described in embodiments herein, both singly and in combination.

In some embodiments, the present invention provides a compound selected from any of formulae II-a, II-b, II-c, II-d, II-e, II-f, II-g, II-h, II-i, II-j, II-k, II-l, II-m, II-n, II-o, II-p, II-q, II-r, II-s, II-t and II-u:

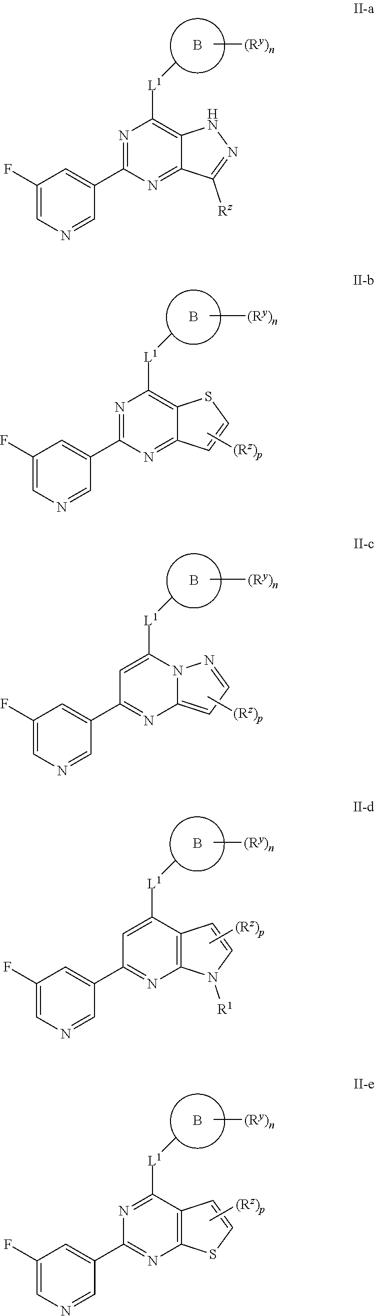

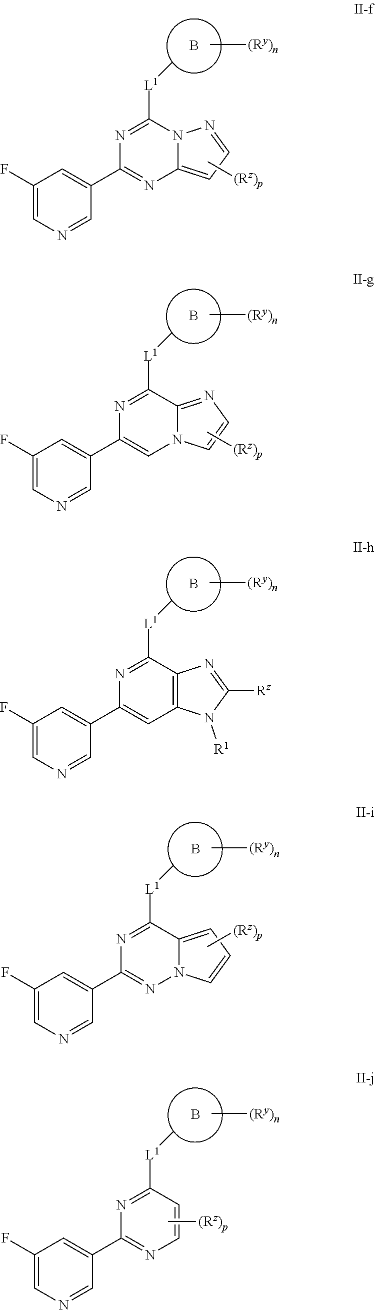

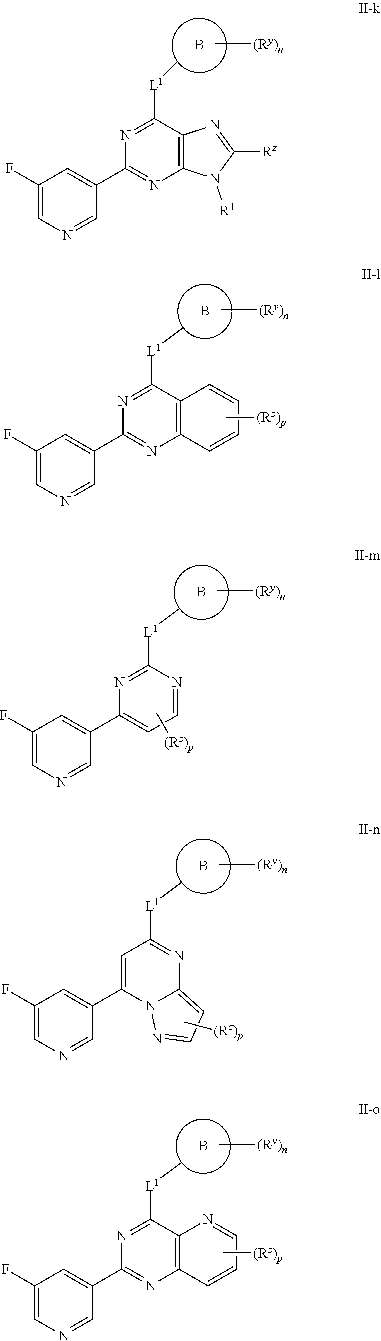

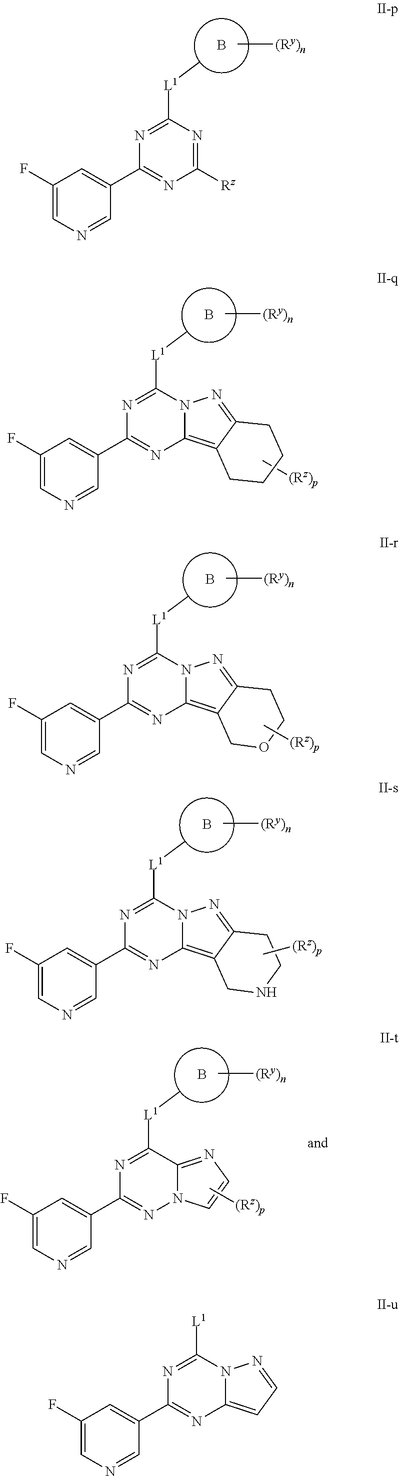

or a pharmaceutically acceptable salt thereof; wherein each variable is as defined herein and described in embodiments for formula I, supra, or described in embodiments herein, both singly and in combination.

In some embodiments, the present invention provides a compound selected from any of formulae III-a, III-b, III-c, III-d, III-e, III-f, III-g, III-h, III-i, III-j, III-k, III-l, III-m, III-n, III-o, III-p, III-q, III-r, III-s, III-t, III-u, III-v, III-w, III-x, III-y, III-z, III-aa, III-bb, III-cc, III-dd, III-ee, III-ff, III-gg, III-hh, III-ii, III-jj and III-kk:

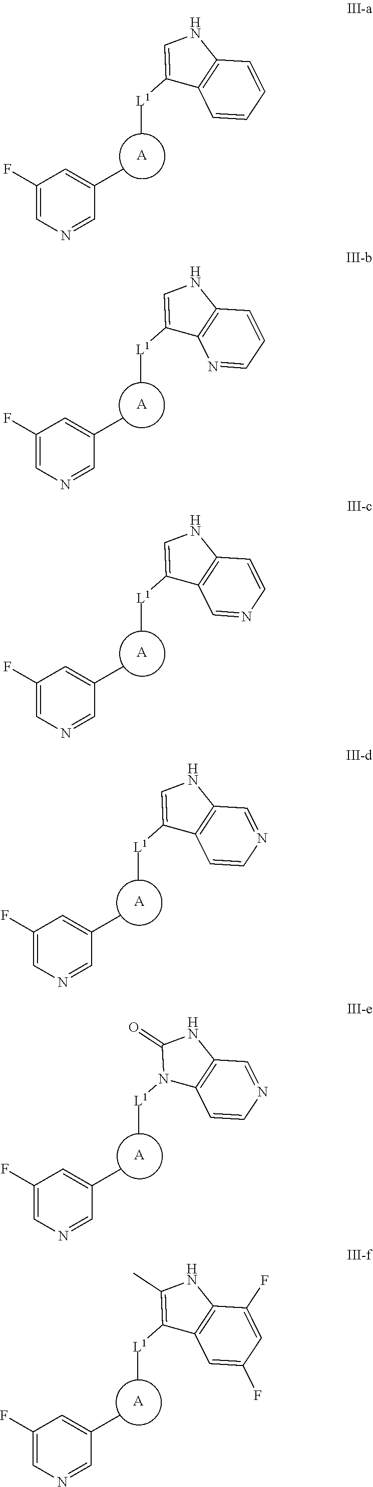

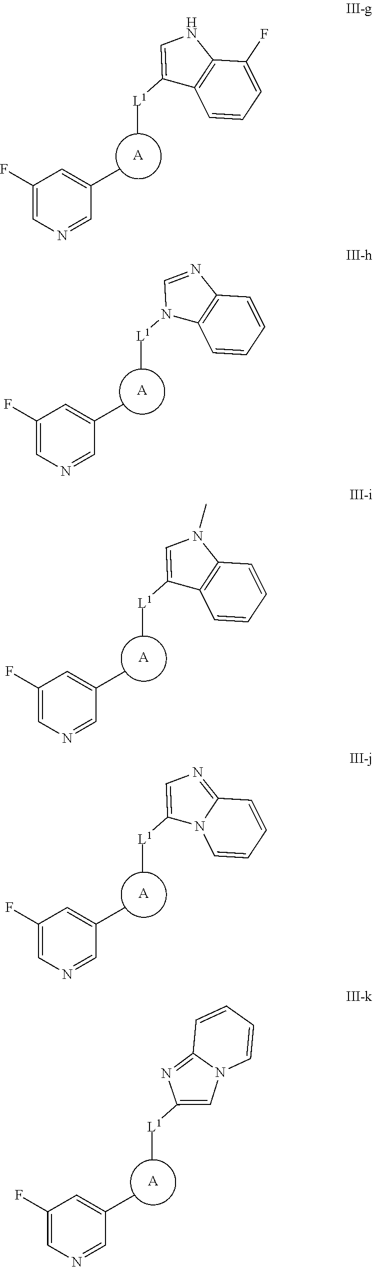

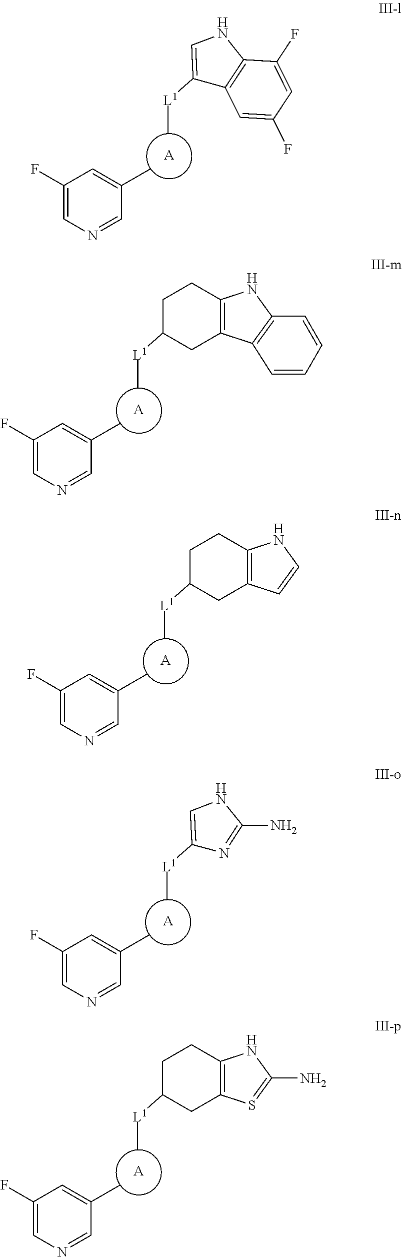

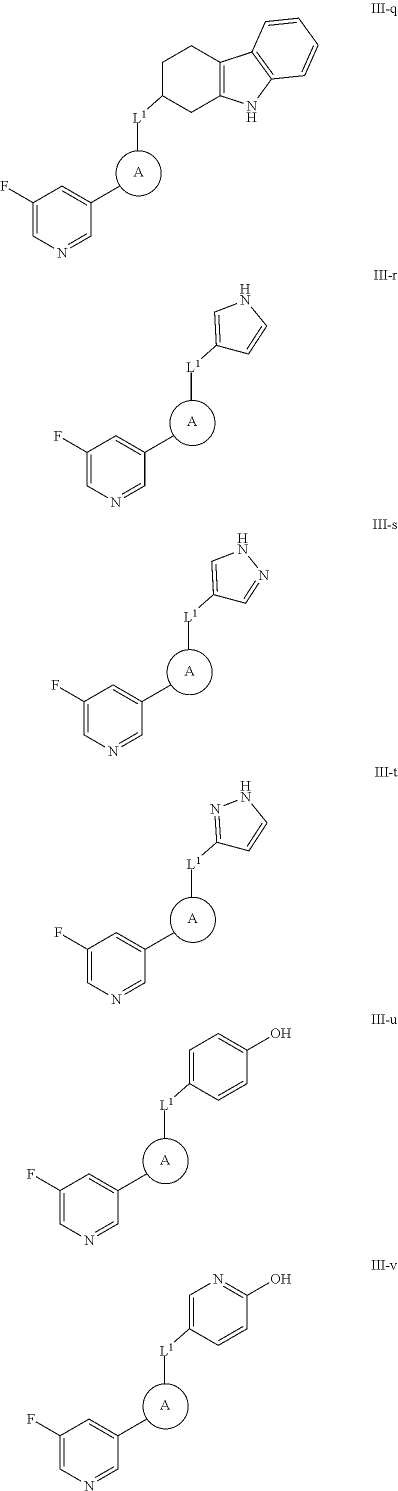

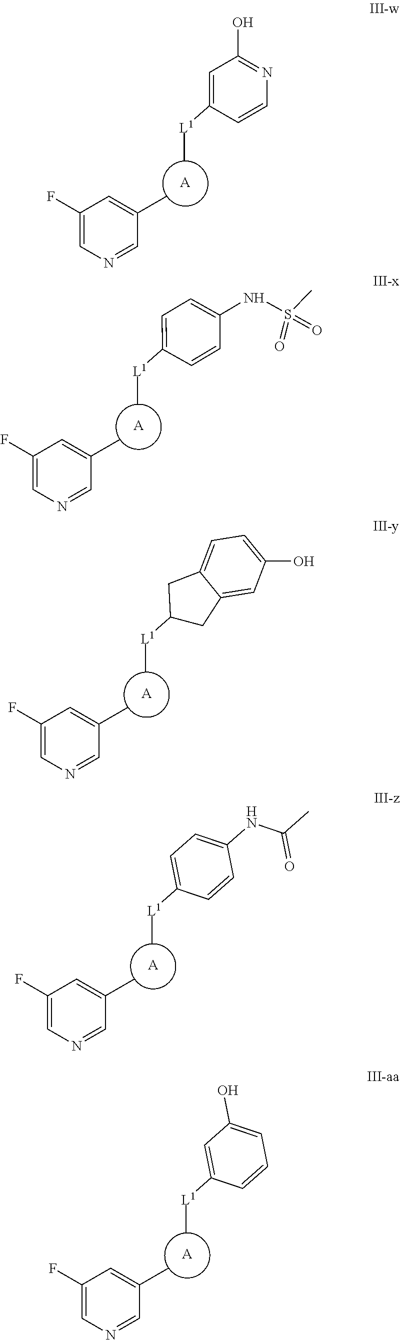

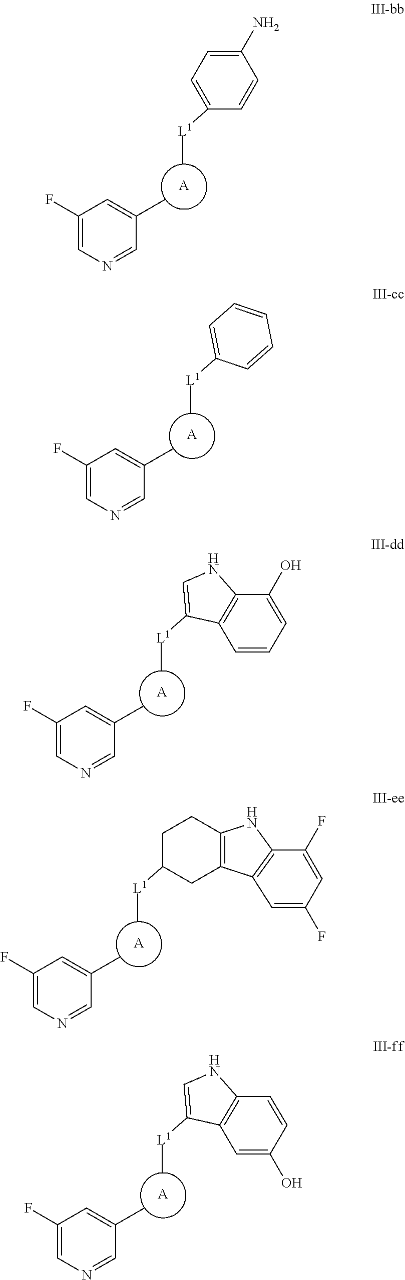

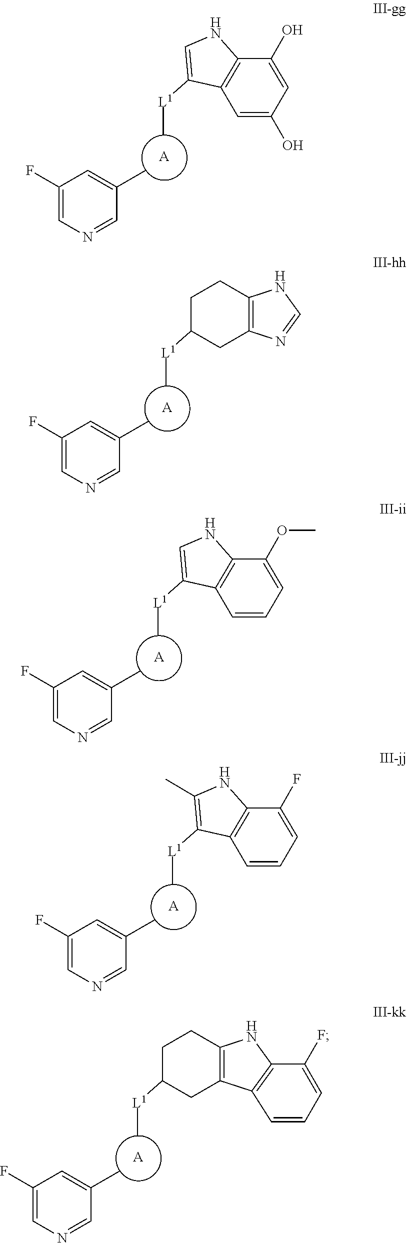

or a pharmaceutically acceptable salt thereof, wherein each variable is as defined herein and described in embodiments for formula I, supra, or described in embodiments herein, both singly and in combination.

In some embodiments, the present invention provides a compound selected from any of formulae IV-a, IV-b, IV-c, IV-d, IV-e, IV-f, IV-g, IV-h, IV-i, IV-j, IV-k, IV-l, IV-m, IV-n, IV-o, IV-p and IV-q:

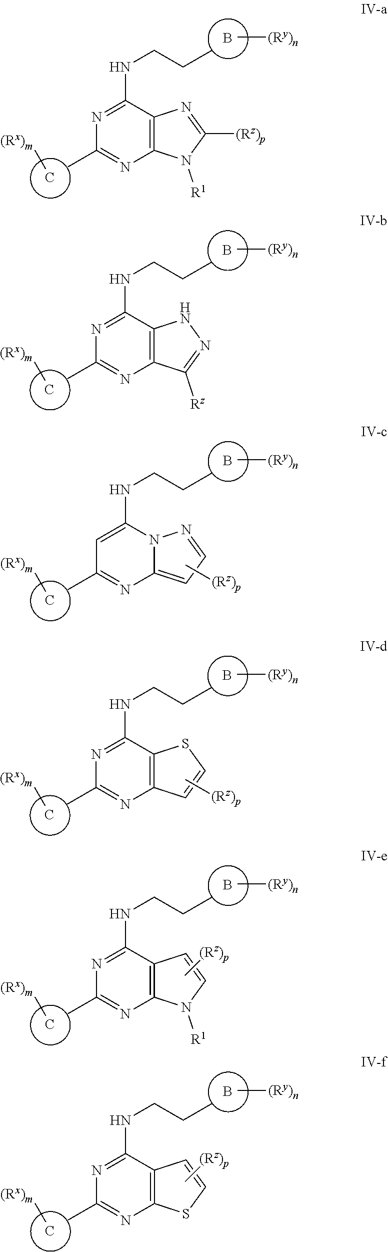

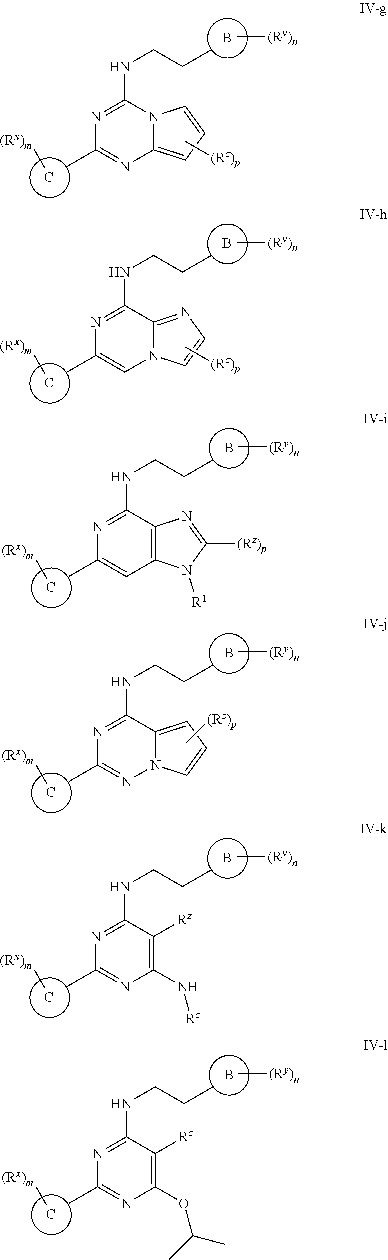

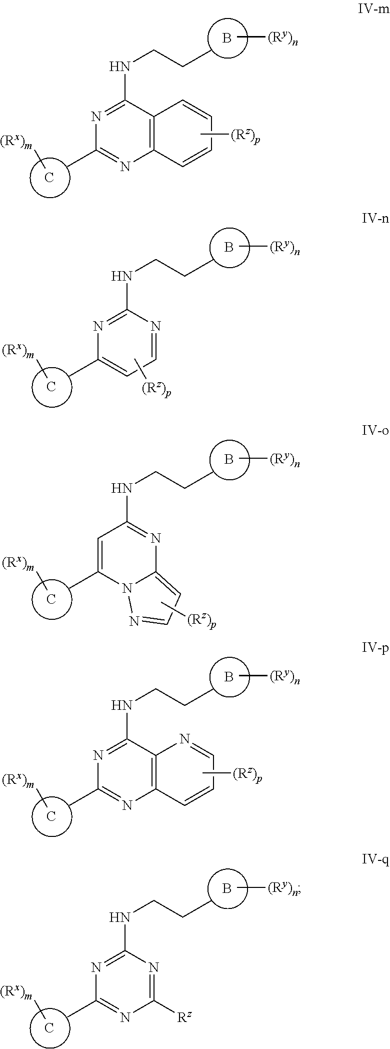

or a pharmaceutically acceptable salt thereof, wherein each variable is as defined herein and described in embodiments for formula I, supra, or described in embodiments herein, both singly and in combination.

In some embodiments, the present invention provides a compound selected from any of formulae V-a, V-b, V-c, V-d, V-e, V-f, V-g, V-h, V-i, V-j, V-k and V-l:

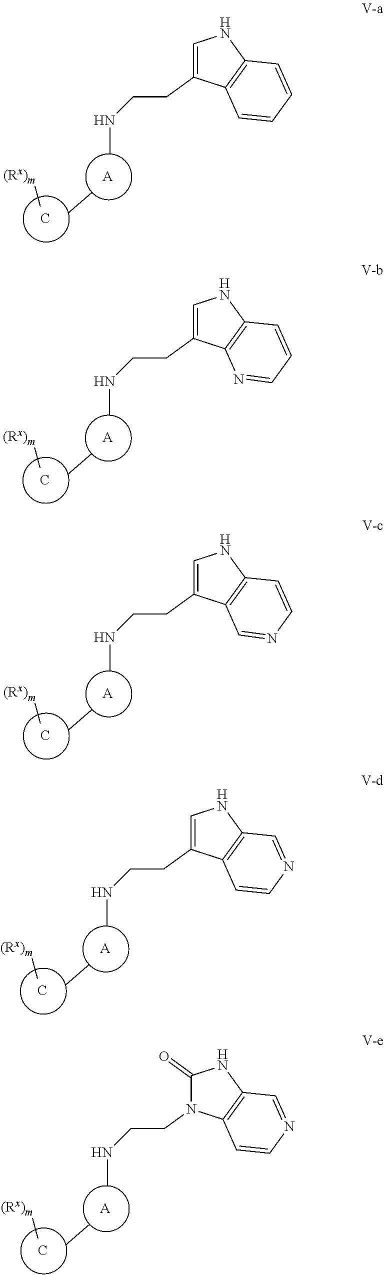

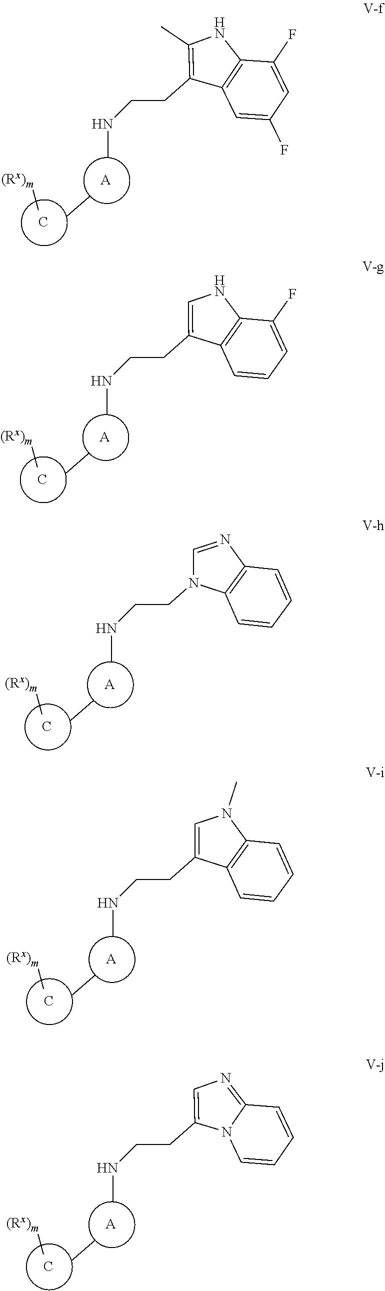

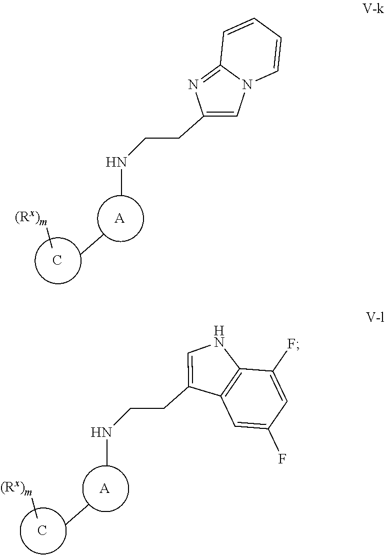

or a pharmaceutically acceptable salt thereof, wherein each variable is as defined herein and described in embodiments for formula I, supra, or described in embodiments herein, both singly and in combination.

In some embodiments, the present invention provides a compound selected from any of formulae VI-a, VI-b, VI-c, VI-d, VI-e, VI-f, VI-g, VI-h, VI-i, VI-j and VI-k:

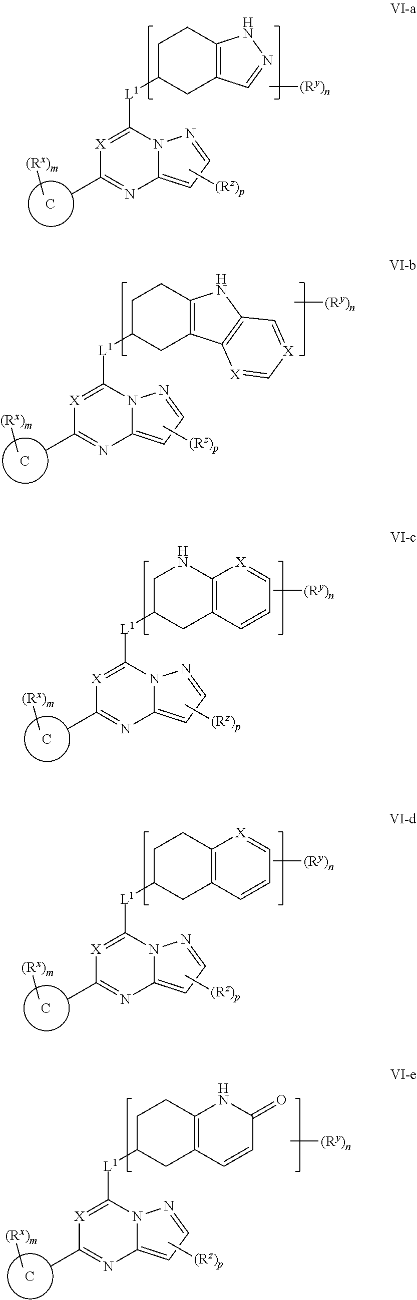

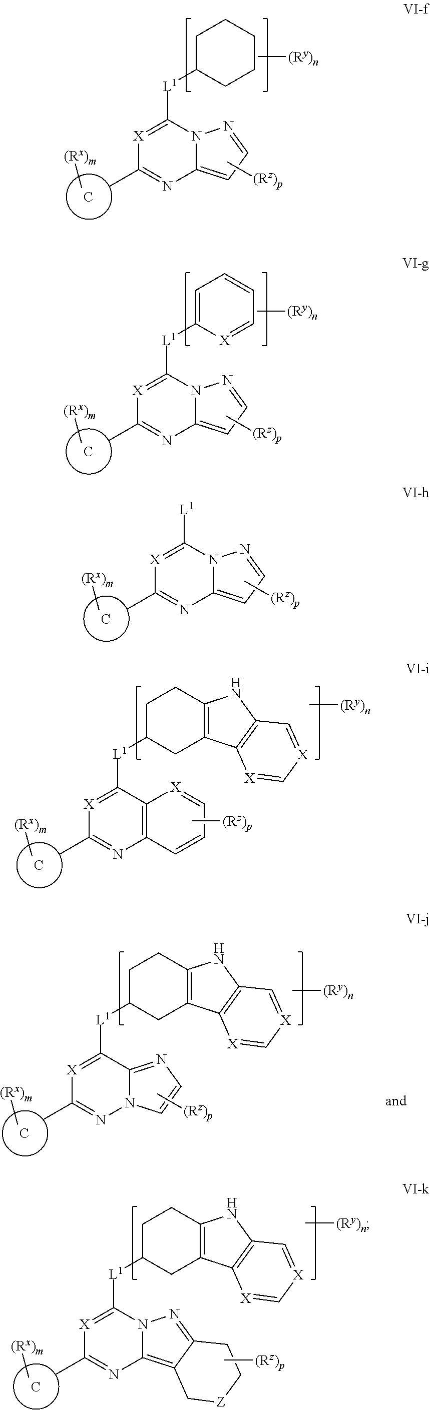

or a pharmaceutically acceptable salt thereof wherein X is N or CH and Z is CH₂, NH or O; wherein each variable is as defined herein and described in embodiments for formula I, supra, or described in embodiments herein, both singly and in combination.

In some embodiments, the present invention provides a compound selected from any of formulae VII-a, VII-b, VII-c, VII-d, VII-e, VII-f, VII-g, VII-h, VII-i and VII-j:

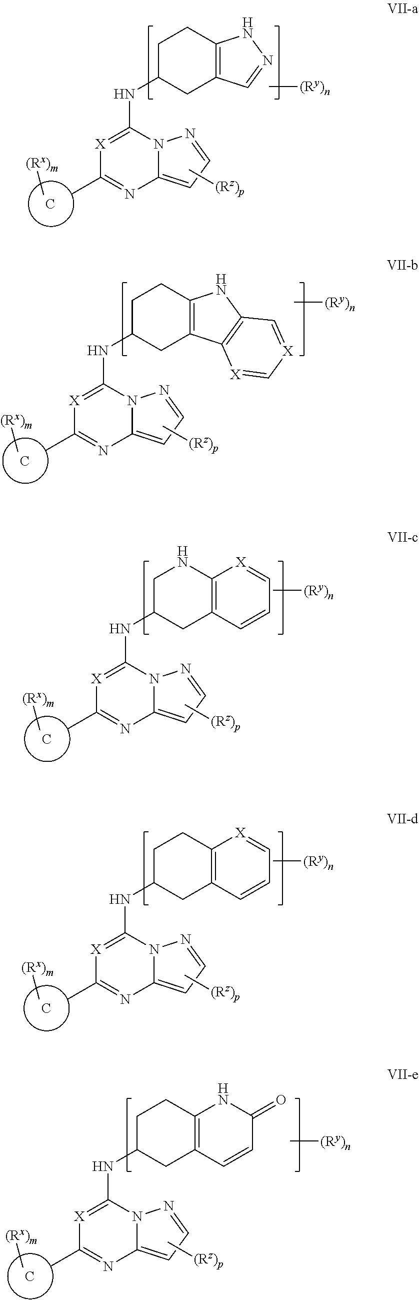

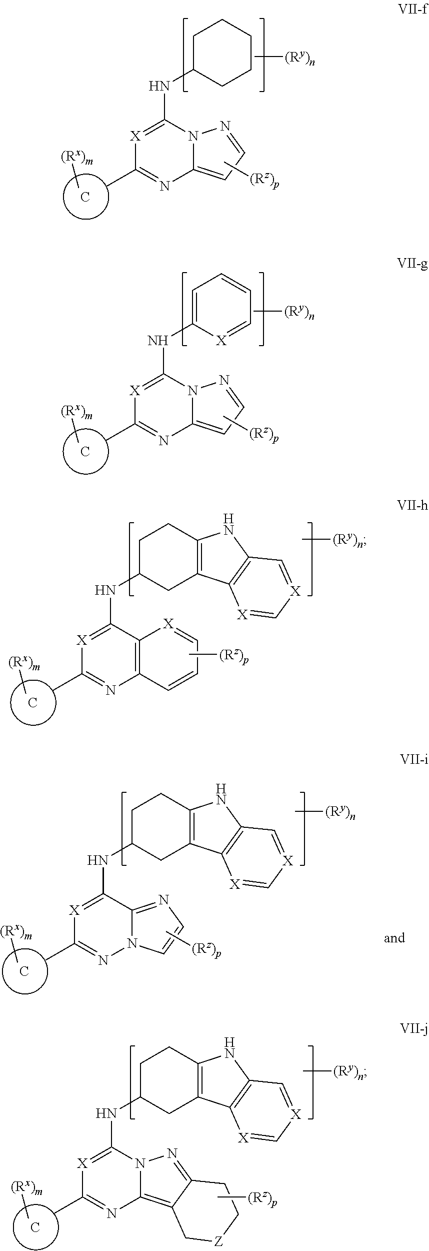

or a pharmaceutically acceptable salt thereof wherein X is N or CH and Z is CH₂, NH or O; wherein each variable is as defined herein and described in embodiments for formula I, supra, or described in embodiments herein, both singly and in combination.

In some embodiments, the present invention provides a compound selected from any of formulae VIII-a, VIII-b, VII-c, VIII-d, VIII-e, VIII-f, VII-g, VIII-h, VIII-i, VIII-j, VIII-k, VIII-l, VIII-m, VIII-n, VIII-o, VIII-p, VIII-q and VIII-r:

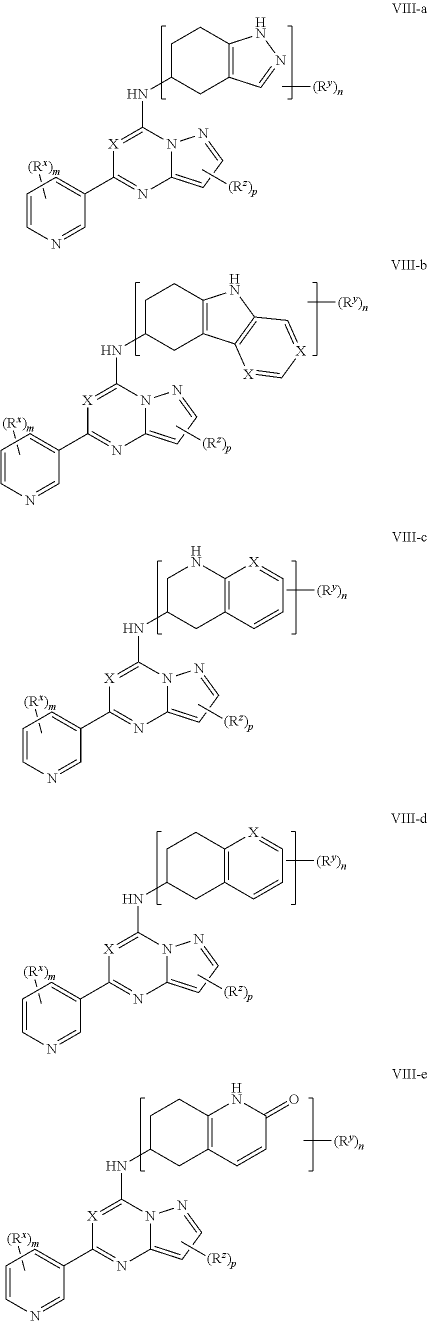

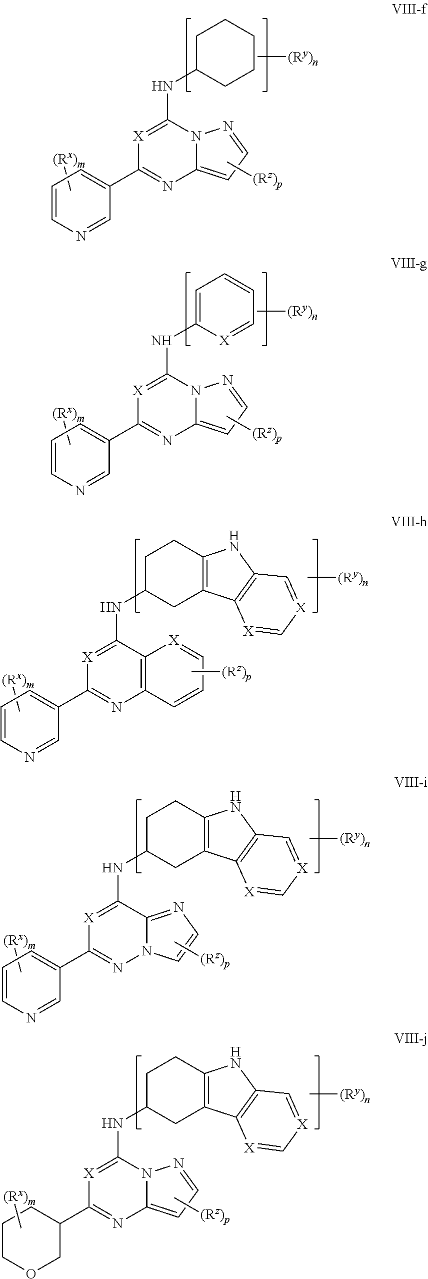

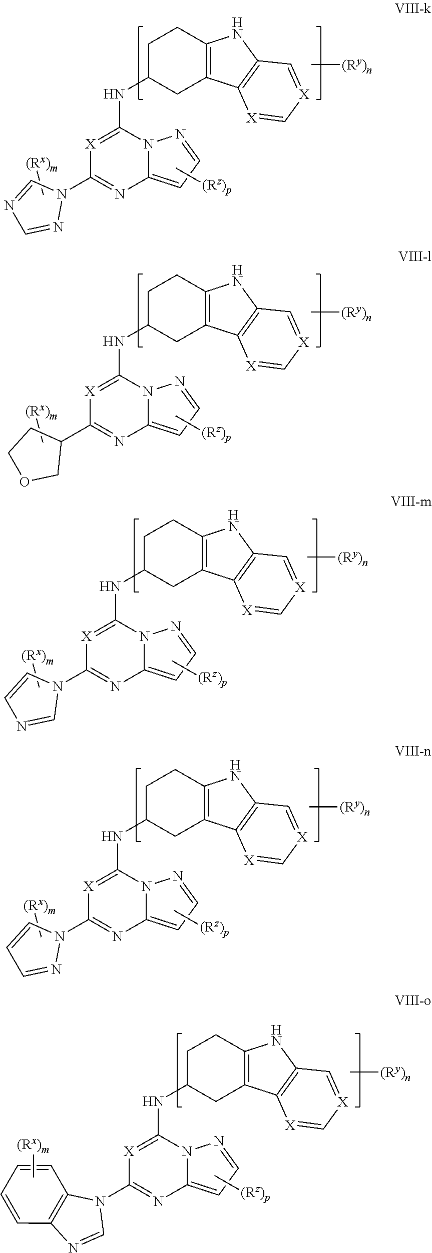

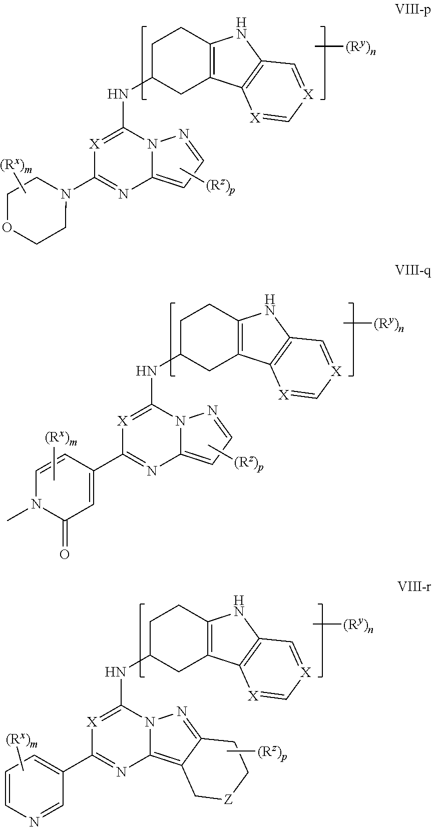

or a pharmaceutically acceptable salt thereof wherein X is N or CH and Z is CH₂, NH or O; wherein each variable is as defined herein and described in embodiments for formula I, supra, or described in embodiments herein, both singly and in combination.

In some embodiments, the present invention provides a compound selected from any of formulae IX-a, IX-b, IX-c, IX-d, IX-e, IX-f, IX-g, IX-h, IX-i, IX-j, IX-k, IX-l, IX-m, IX-n, IX-o, IX-p, IX-q, IX-r, IX-s and IX-t:

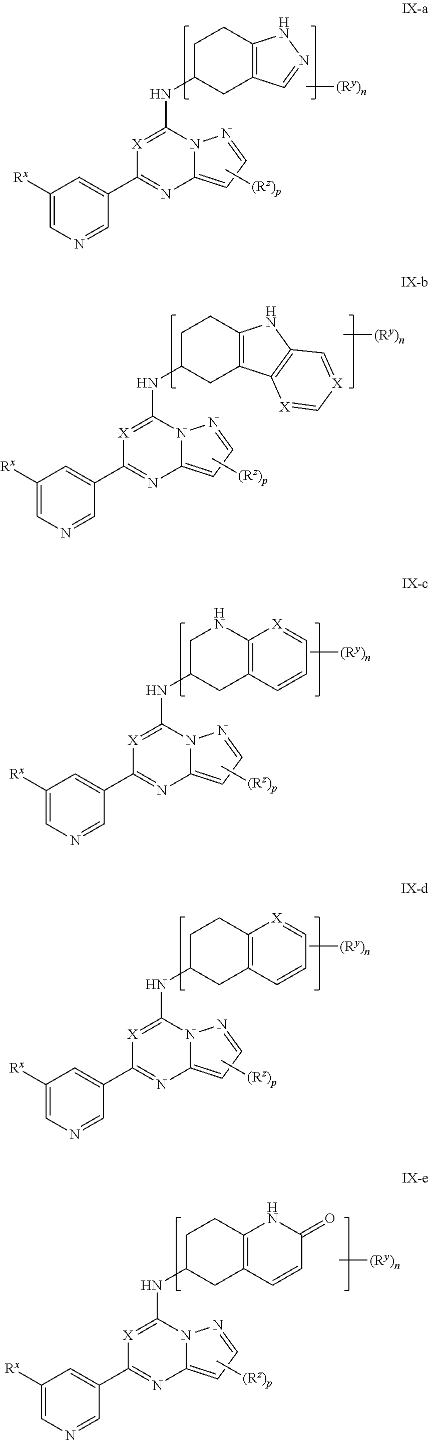

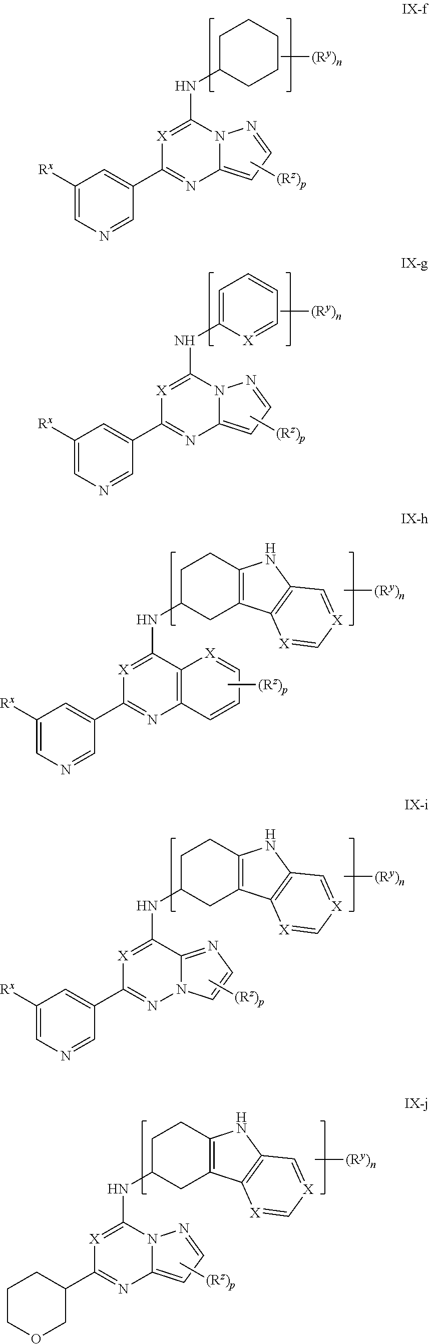

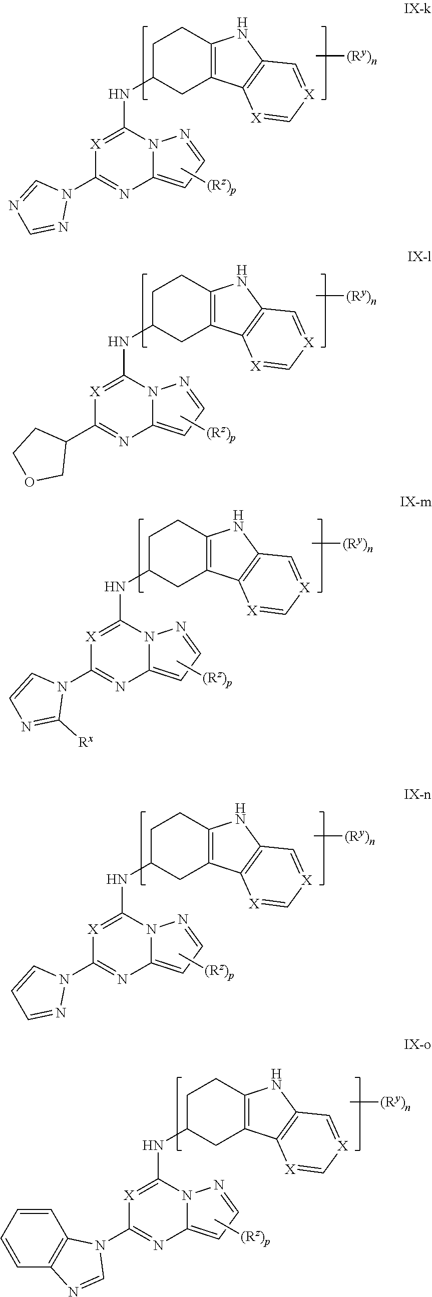

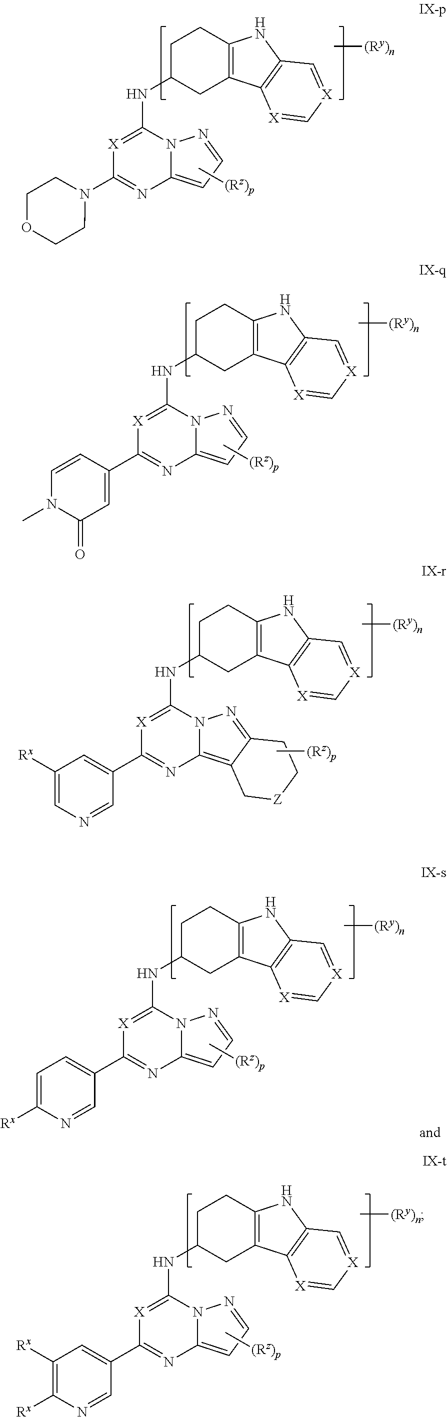

or a pharmaceutically acceptable salt thereof wherein X is N or CH and Z is CH₂, NH or O; wherein each variable is as defined herein and described in embodiments for formula I, supra, or described in embodiments herein, both singly and in combination.

In some embodiments, the present invention provides a compound selected from any of formulae X-a, X-b, X-c, X-d, X-e, X-f, X-g, X-h, X-i, X-j, X-k, X-l, X-m, X-n, X-o, X-p, X-q, X-r, X-s, X-t and X-u:

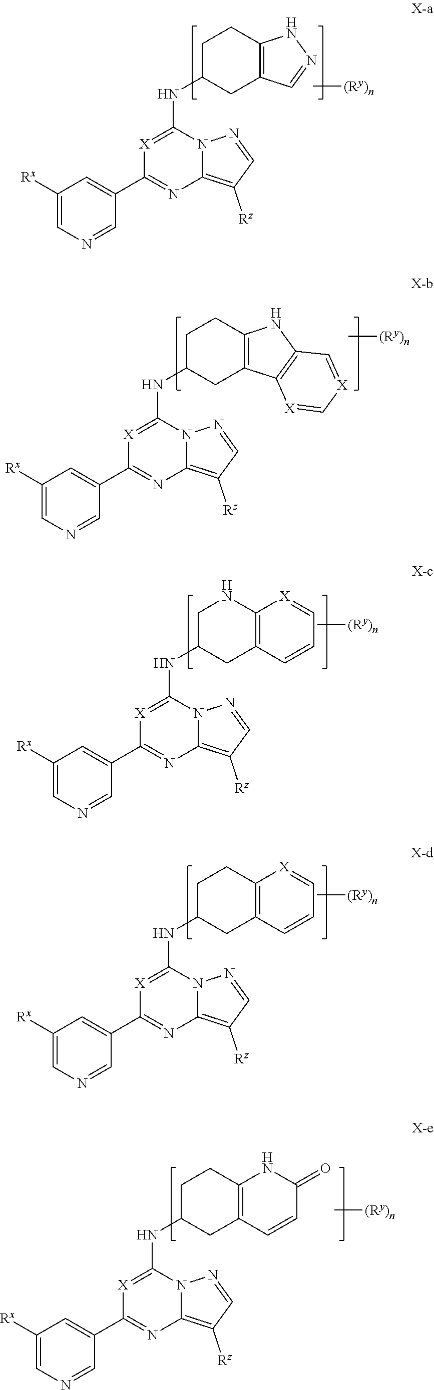

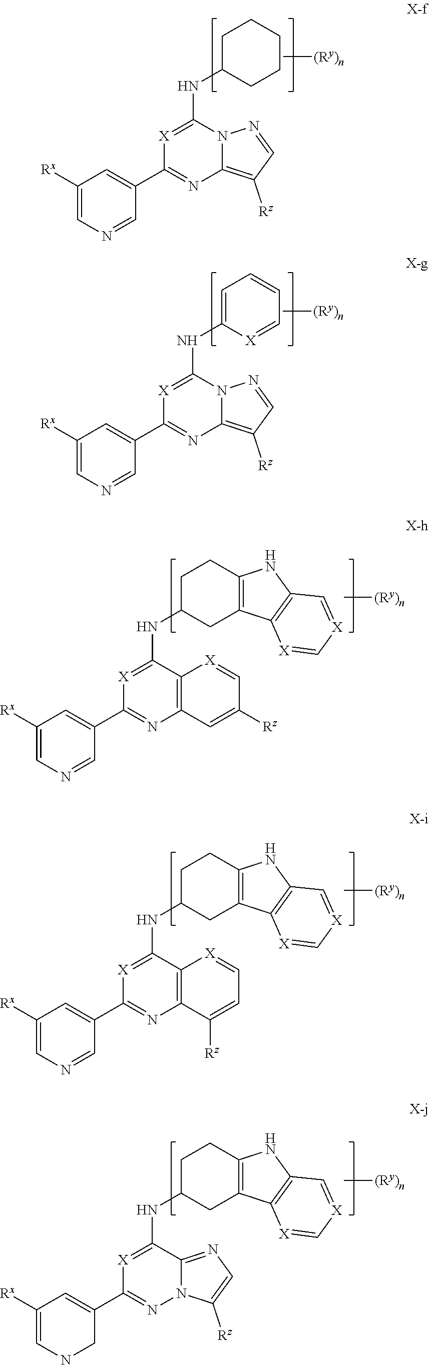

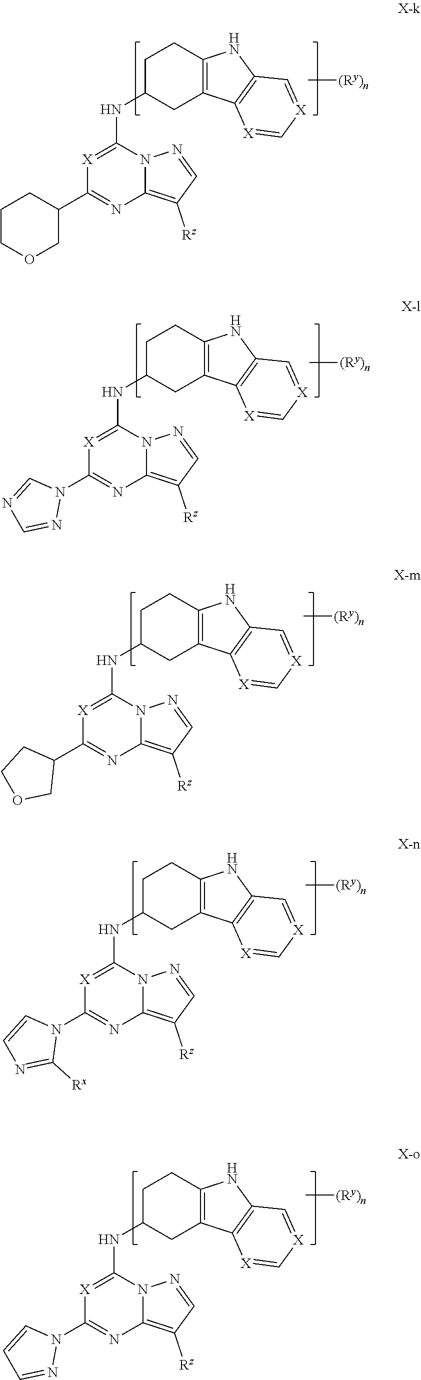

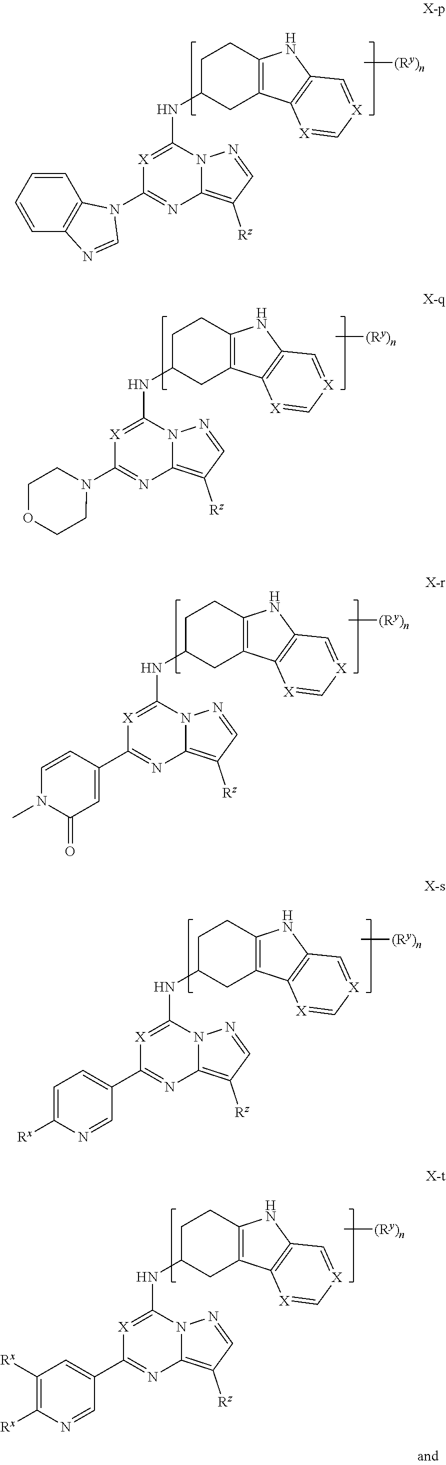

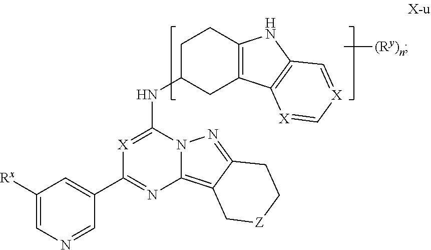

or a pharmaceutically acceptable salt thereof wherein X is N or CH and Z is CH₂, NH or O; wherein each variable is as defined herein and described in embodiments for formula I, supra, or described in embodiments herein, both singly and in combination.

In some embodiments, the present invention provides a compound selected from any of formulae XI-a, XI-b, XI-c and XI-d:

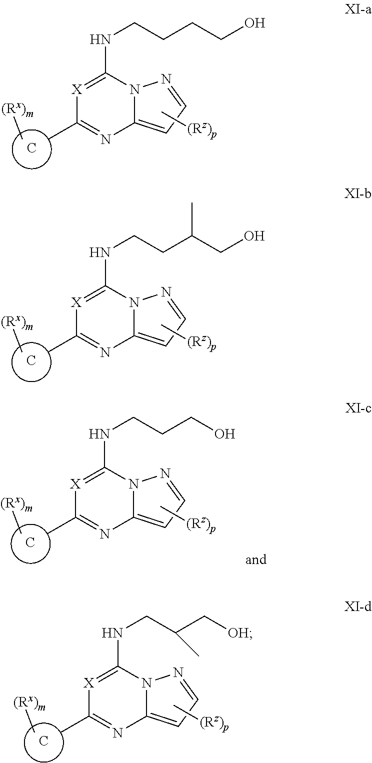

or a pharmaceutically acceptable salt thereof wherein X is N or CH; wherein each variable is as defined herein and described in embodiments for formula I, supra, or described in embodiments herein, both singly and in combination.

In some embodiments, the present invention provides a compound of formula I provided that when Ring A is

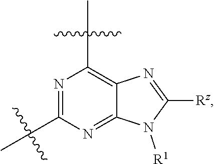

### Ring B is not

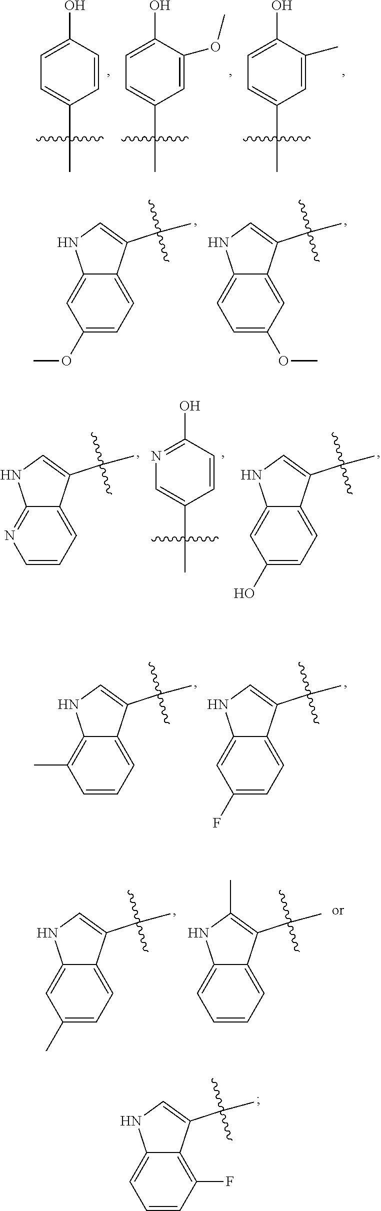

and/or RC is not

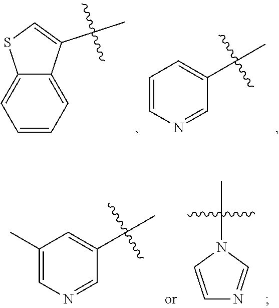

and/or R¹ is not

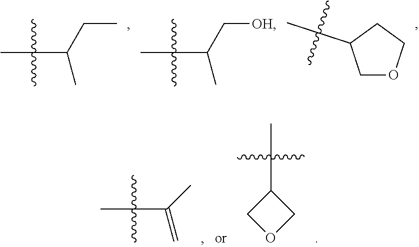

In some embodiments, the present invention provides a compound of formula I provided that L¹ is not —NHCH₂CH₂—. In some embodiments, the present invention provides a compound of formula I provided that when Ring A is

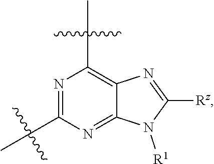

L¹ is not —NHCH₂CH₂—.

Exemplary compounds of the present invention are set forth in Table 1, below:

TABLE 1
Exemplary Compounds of Formula I

| | |
|---|---|
| 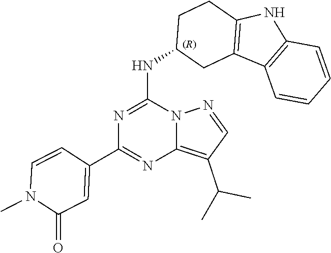 | I-1 |
| 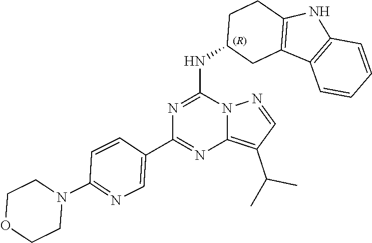 | I-2 |
| 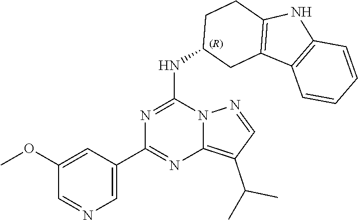 | I-3 |
| 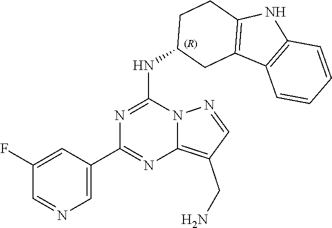 | I-4 |
| 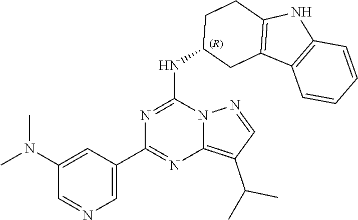 | I-5 |
| 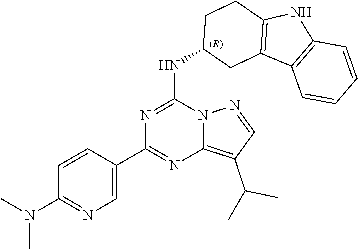 | I-6 |
| 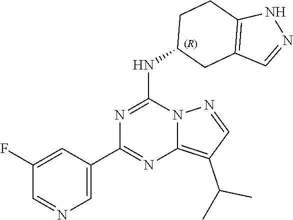 | I-7a |
| 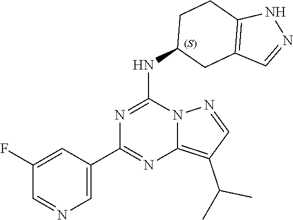 | I-7b |
| 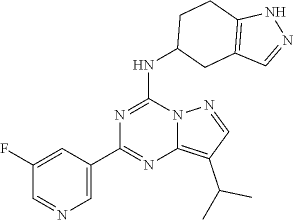 [SMILES: CC(C)c1cnn2c(NC3CCc4[nH]ncc4C3)nc(-c3cncc(F)c3)nc12] | I-7c |
| 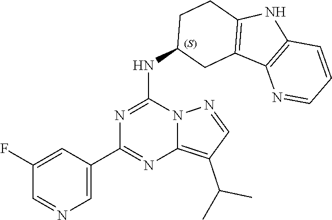 | I-8a |
| 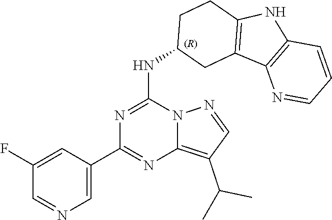 | I-8b |
| 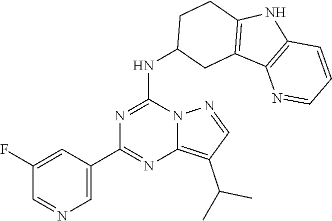 [SMILES: CC(C)c1cnn2c(NC3CCc4[nH]c5cccnc5c4C3)nc(-c3cncc(F)c3)nc12] | I-8c |
| 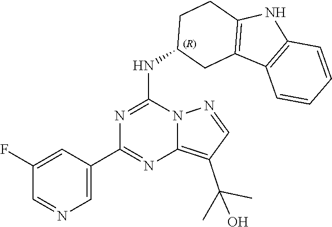 | I-9 |
| 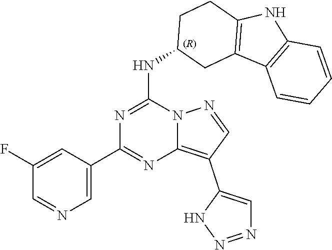 | I-10 |
| 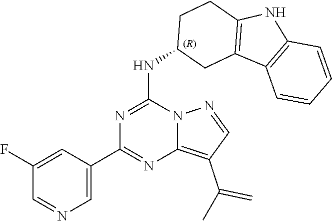 | I-11 |
| 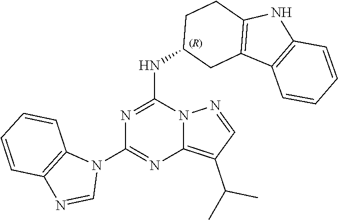 | I-12 |
| 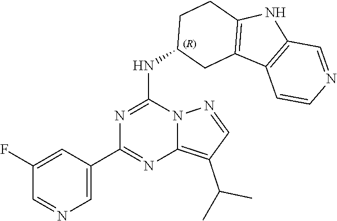 | I-13a |
| 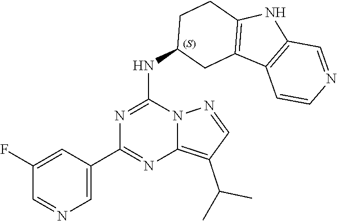 | I-13b |
| 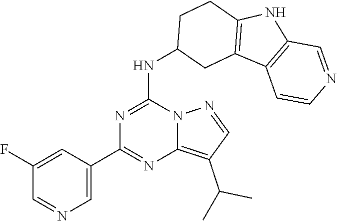 [SMILES: CC(C)c1cnn2c(NC3CCc4[nH]c5cnccc5c4C3)nc(-c3cncc(F)c3)nc12] | I-13c |
| 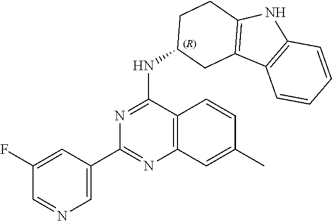 | I-14 |
| 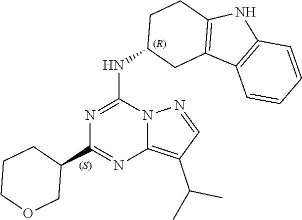 | I-15a |
| 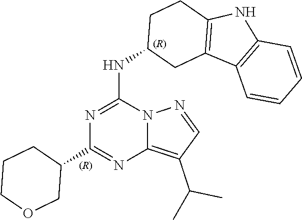 | I-15b |
| 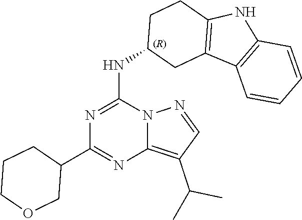 | I-15c |
| 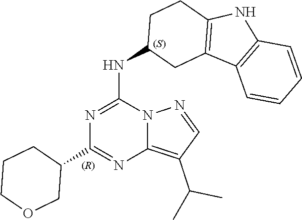 | I-15d |
| 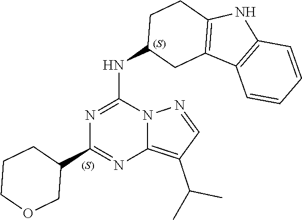 | I-15e |
| 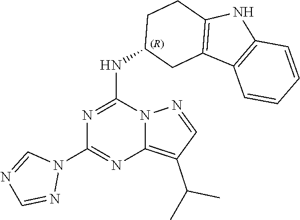 | I-16 |
| 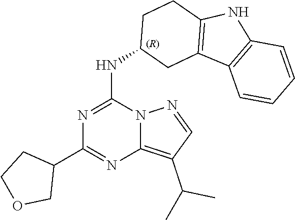 | I-17 |
| 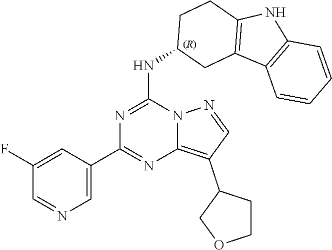 | I-18 |
| 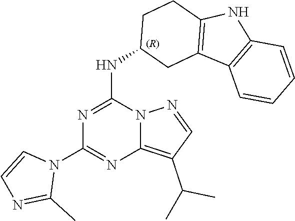 | I-19 |
| 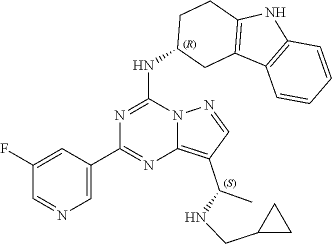 | I-20a |
| 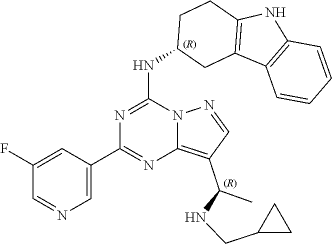 | I-20b |
| 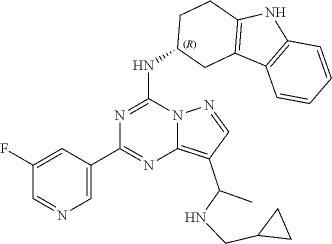 | I-20c |
| 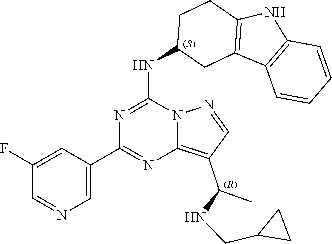 | I-20d |
| 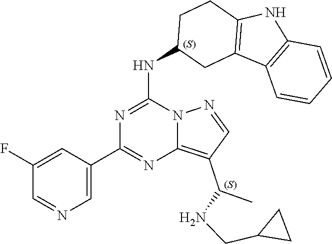 | I-20e |
| 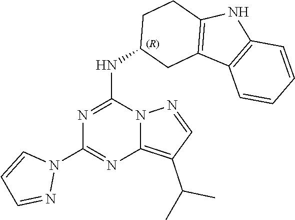 | I-21 |
| 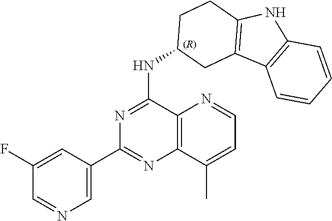 | I-22 |
| 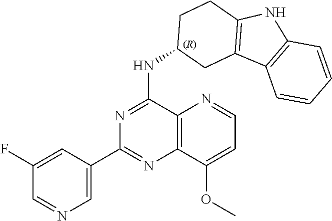 | I-23 |
| 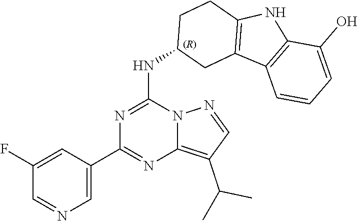 | I-24a |
| 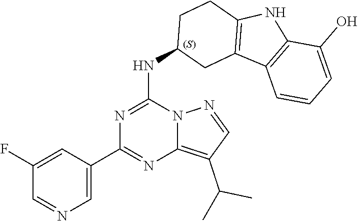 | I-24b |
| 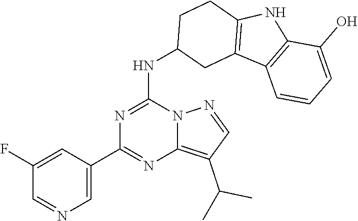 [SMILES: CC(C)c1cnn2c(NC3CCc4[nH]c5c(O)cccc5c4C3)nc(-c3cncc(F)c3)nc12] | I-24c |
| 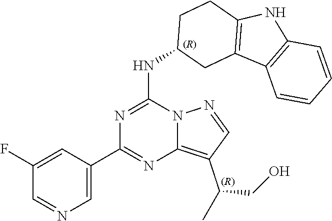 | I-25a |
| 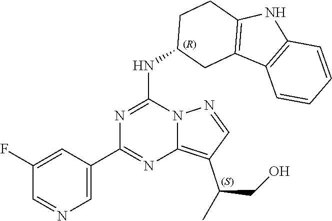 | I-25b |
| 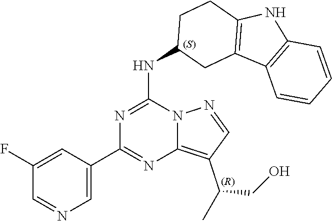 | I-25c |
| 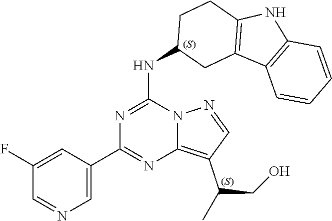 | I-25d |
| 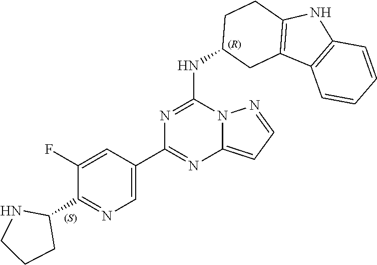 | I-26a |
| 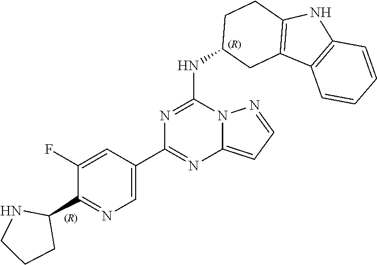 | I-26b |
| 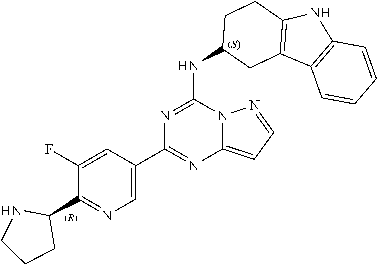 | I-26c |
| 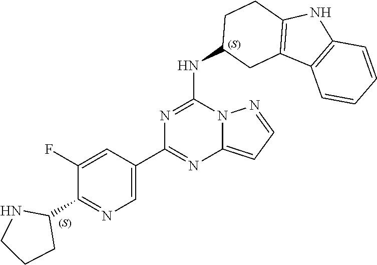 | I-26d |
| 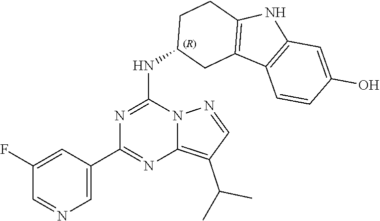 | I-27a |
| 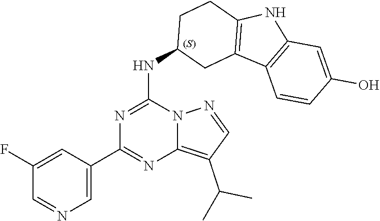 | I-27b |
| 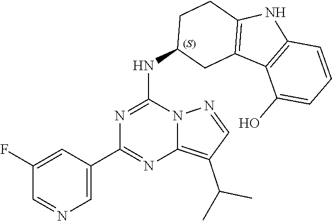 | I-29a |
| 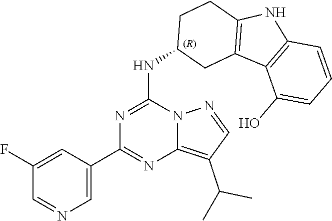 | I-29b |
| 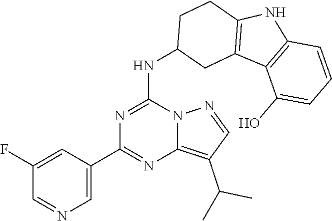 [SMILES: CC(C)c1cnn2c(NC3CCc4[nH]c5cccc(O)c5c4C3)nc(-c3cncc(F)c3)nc12] | I-29c |
| 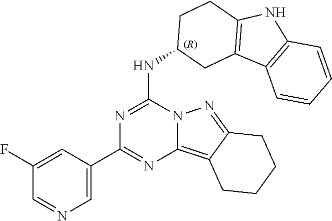 | I-30 |
| 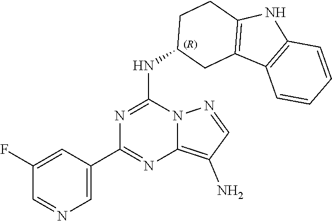 | I-31 |
| 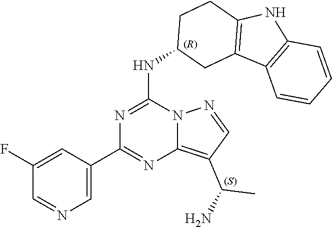 | I-32a |
| 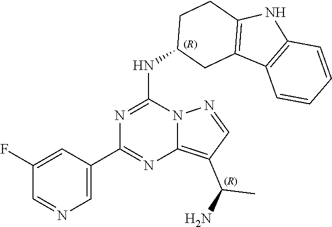 | I-32b |
| 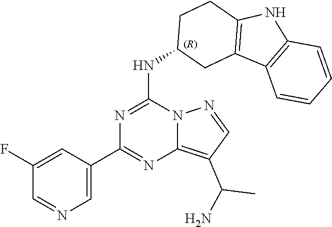 | I-32c |
| 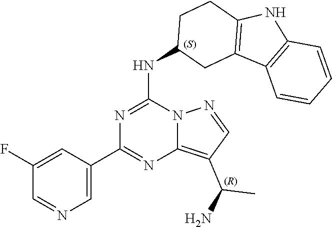 | I-32d |
| 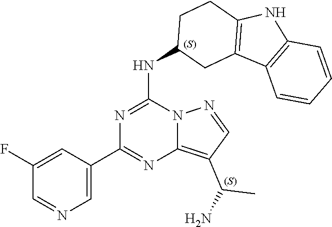 | I-32e |
| 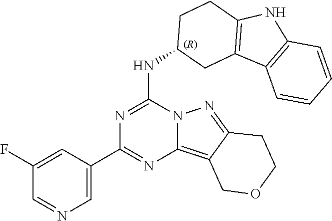 | I-33 |
| 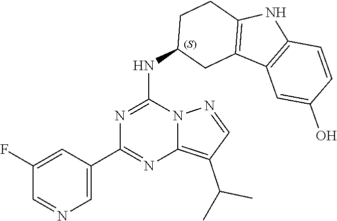 | I-34a |
| 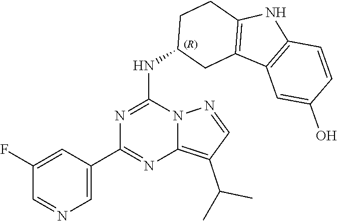 | I-34b |
| 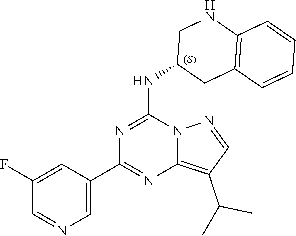 | I-35a |
| 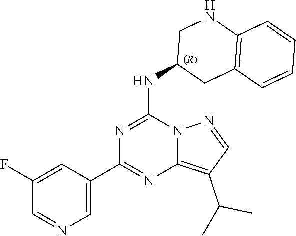 | I-35b |
| 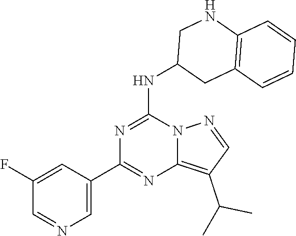 [SMILES: CC(C)c1cnn2c(NC3CNc4ccccc4C3)nc(-c3cncc(F)c3)nc12] | I-35c |
| 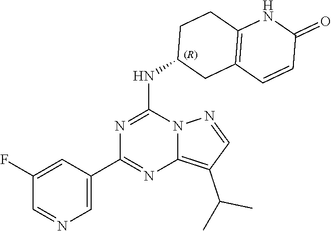 | I-36a |
| 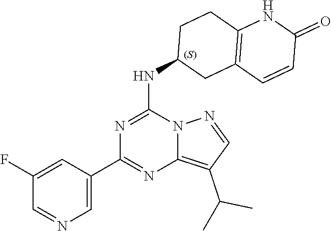 | I-36b |
| 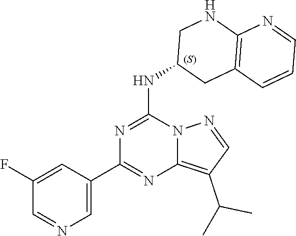 | I-37a |
| 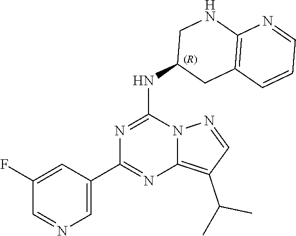 | I-37b |
| 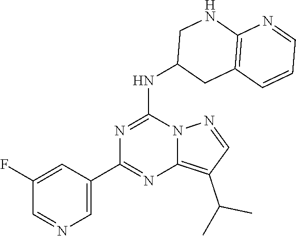 [SMILES: CC(C)c1cnn2c(NC3CNc4ncccc4C3)nc(-c3cncc(F)c3)nc12] | I-37c |
| 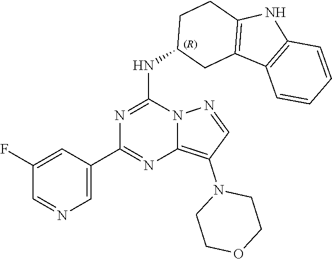 | I-38 |
| 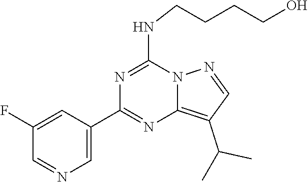 [SMILES: CC(C)c1cnn2c(NCCCCO)nc(-c3cncc(F)c3)nc12] | I-39 |
| 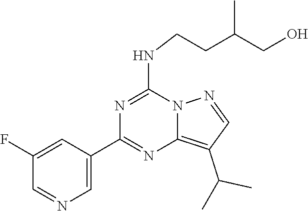 [SMILES: CC(CO)CCNc1nc(-c2cncc(F)c2)nc2c(C(C)C)cnn12] | I-40 |
| 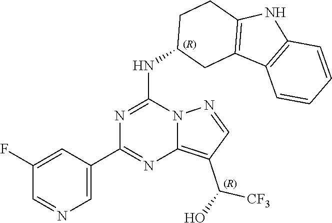 | I-41a |
| 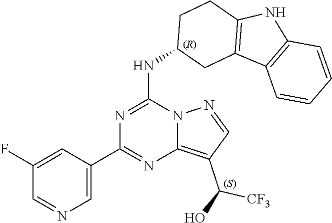 | I-41b |
| 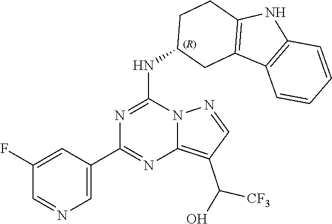 | I-41c |
| 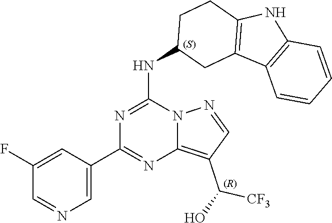 | I-41d |
| 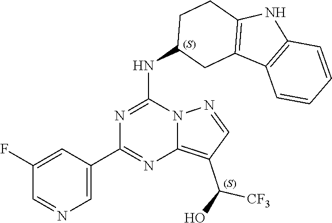 | I-41e |
| 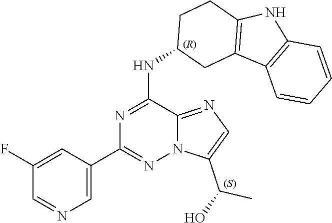 | I-42a |
| 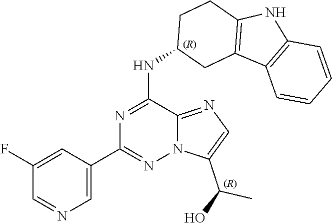 | I-42b |
| 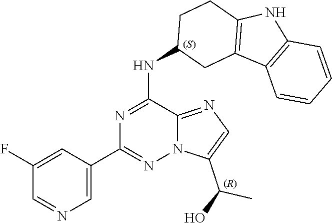 | I-42c |
| 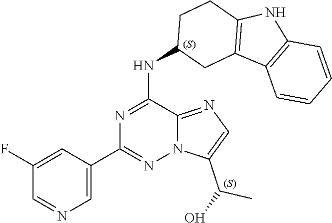 | I-42d |
| 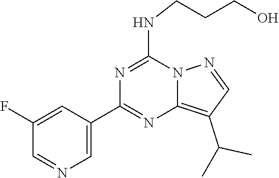 [SMILES: CC(C)c1cnn2c(NCCCO)nc(-c3cncc(F)c3)nc12] | I-43 |
| 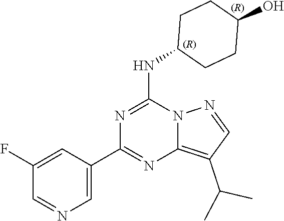 | I-44a |
| 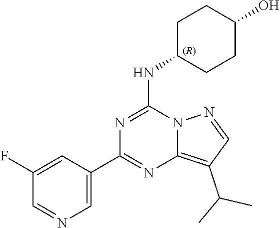 | I-44b |
| 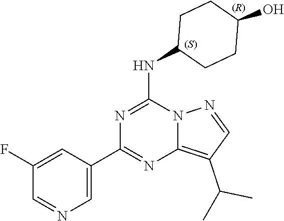 | I-44c |
| 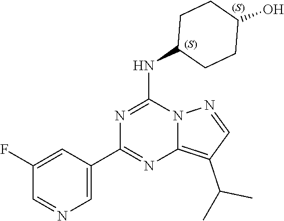 | I-44d |
| 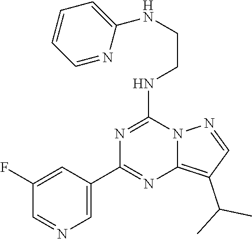 [SMILES: CC(C)c1cnn2c(NCCNc3ccccn3)nc(-c3cncc(F)c3)nc12] | I-45 |
| 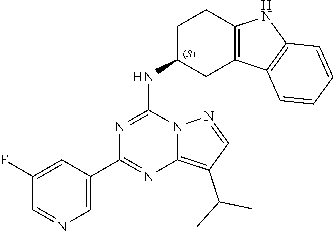 | I-47 |
| 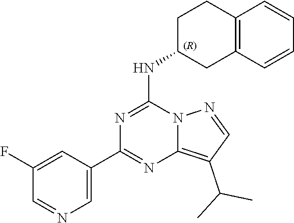 | I-48a |
| 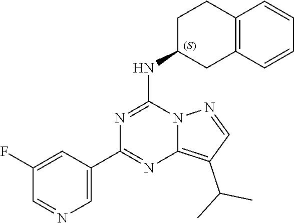 | I-48b |
| 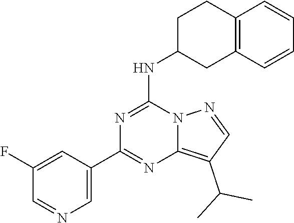 [SMILES: CC(C)c1cnn2c(NC3CCc4ccccc4C3)nc(-c3cncc(F)c3)nc12] | I-48c |
| 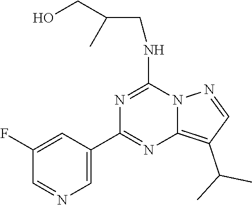 [SMILES: CC(CO)CNc1nc(-c2cncc(F)c2)nc2c(C(C)C)cnn12] | I-49 |
| 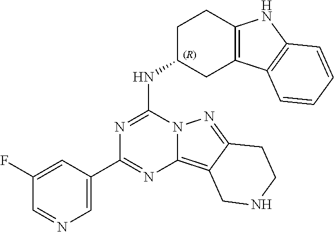 | I-50 |
| 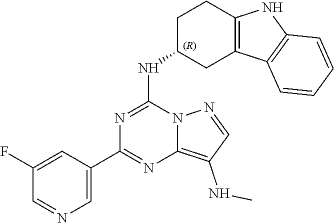 | I-51 |
| 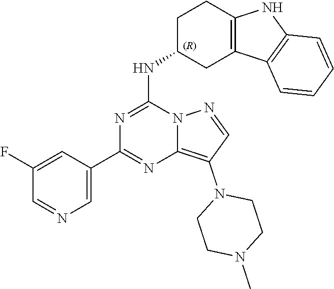 | I-52 |
| 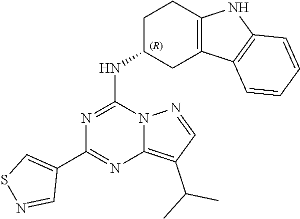 | I-53 |
| 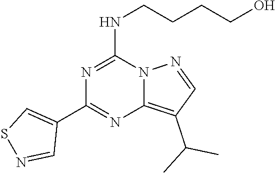 [SMILES: CC(C)c1cnn2c(NCCCCO)nc(-c3cnsc3)nc12] | I-54 |
| 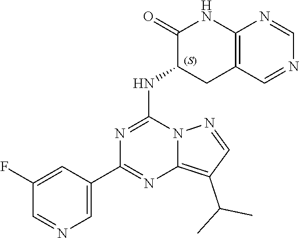 | I-55a |
| 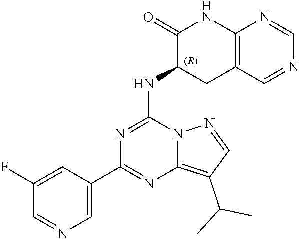 | I-55b |
| 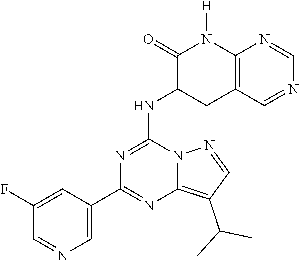 [SMILES: CC(C)c1cnn2c(NC3Cc4cncnc4NC3=O)nc(-c3cncc(F)c3)nc12] | I-55c |
| 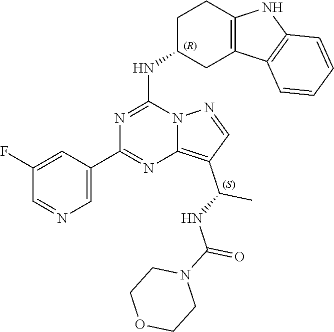 | I-56a |
| 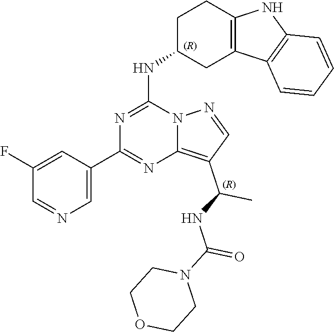 | I-56b |
| 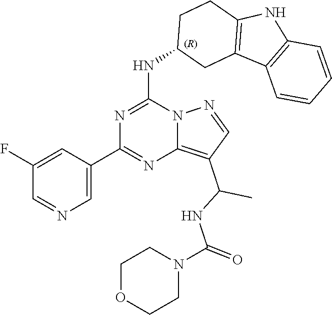 | I-56c |
| 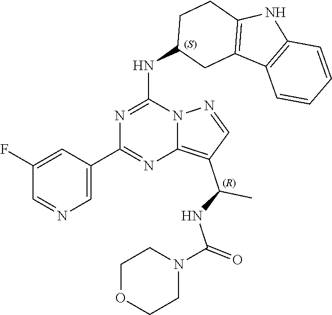 | I-56d |
| 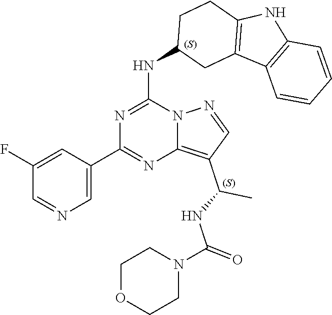 | I-56e |
| 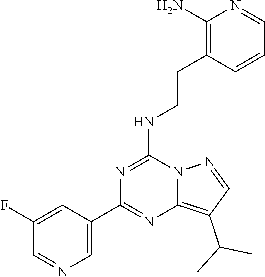 [SMILES: CC(C)c1cnn2c(NCCc3cccnc3N)nc(-c3cncc(F)c3)nc12] | I-58 |
| 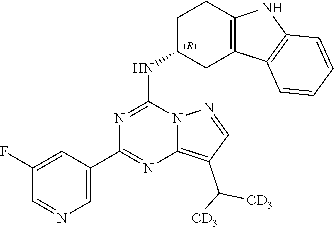 | I-59 |
| 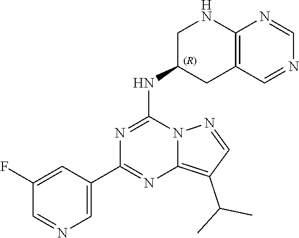 | I-60a |
| 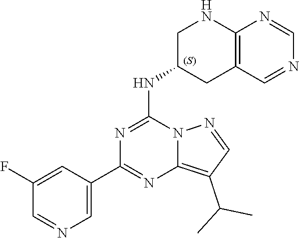 | I-60b |
| 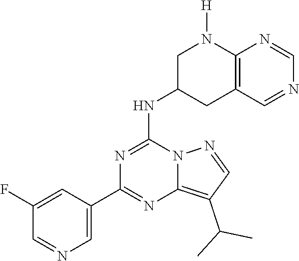 [SMILES: CC(C)c1cnn2c(NC3CNc4ncncc4C3)nc(-c3cncc(F)c3)nc12] | I-60c |
| 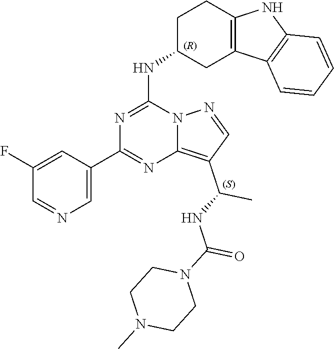 | I-61a |
| 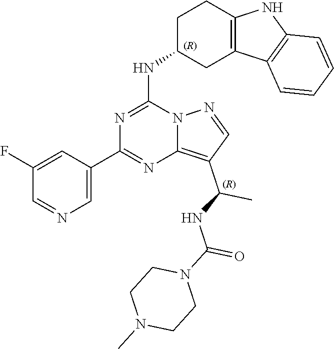 | I-61b |
| 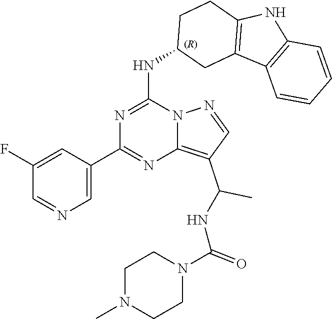 | I-61c |
| 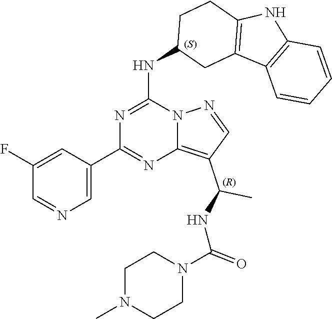 | I-61d |
| 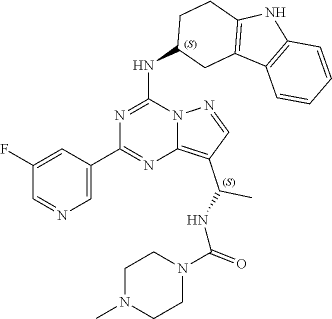 | I-61e |
| 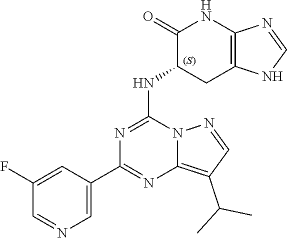 | I-62a |
| 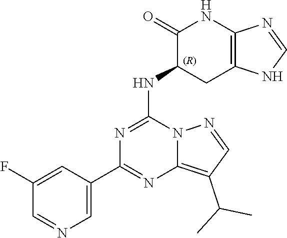 | I-62b |
| 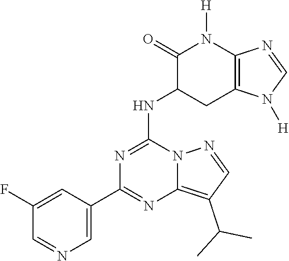 [SMILES: CC(C)c1cnn2c(NC3Cc4[nH]cnc4NC3=O)nc(-c3cncc(F)c3)nc12] | I-62c |
| 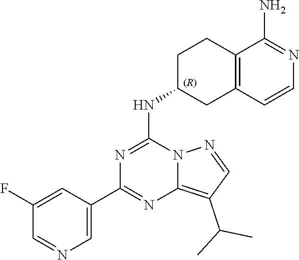 | I-63a |
| 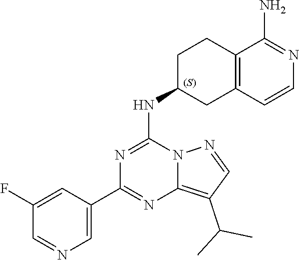 | I-63b |
| 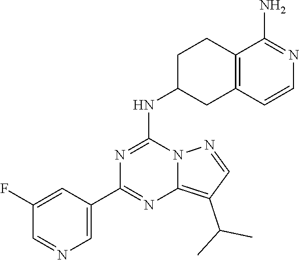 [SMILES: CC(C)c1cnn2c(NC3CCc4c(ccnc4N)C3)nc(-c3cncc(F)c3)nc12] | I-63c |
| 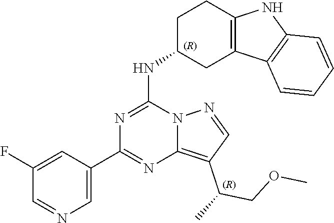 | I-64a |
| 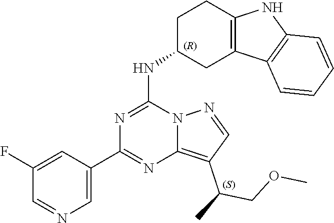 | I-64b |
| 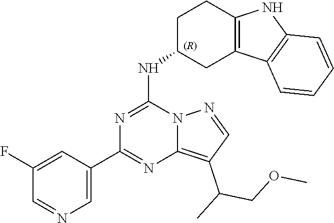 | I-64c |
| 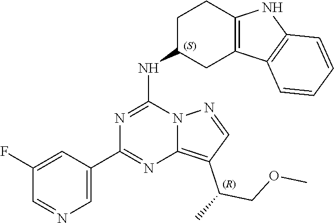 | I-64d |
| 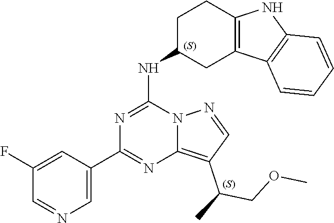 | I-64e |
| 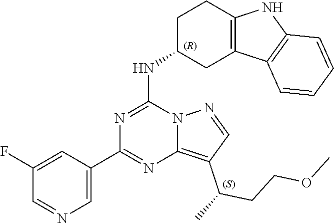 | I-65a |
| 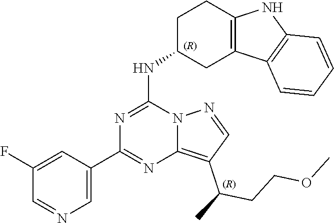 | I-65b |
| 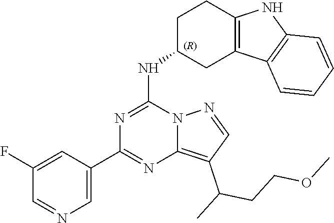 | I-65c |
| 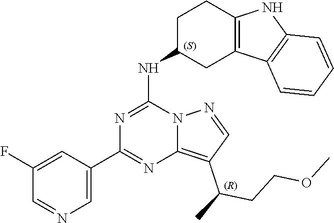 | I-65d |
| 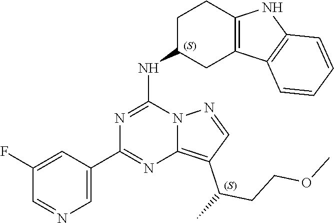 | I-65e |
| 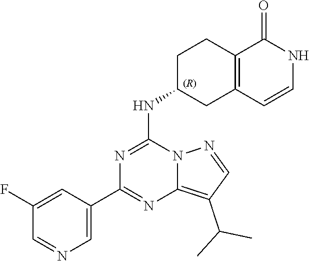 | I-66a |
| 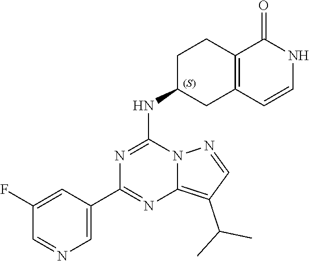 | I-66b |
| 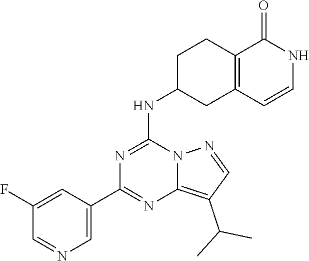 [SMILES: CC(C)c1cnn2c(NC3CCc4c(cc[nH]c4=O)C3)nc(-c3cncc(F)c3)nc12] | I-66c |
| 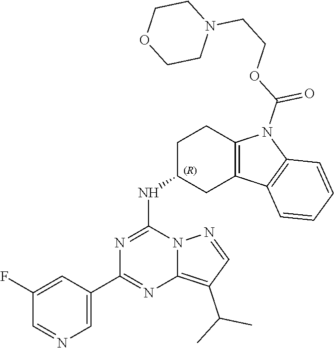 | I-67 |
| 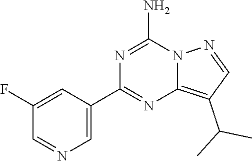 [SMILES: CC(C)c1cnn2c(N)nc(-c3cncc(F)c3)nc12] | I-68 |
| 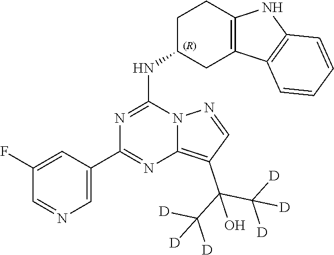 | I-69 |
| 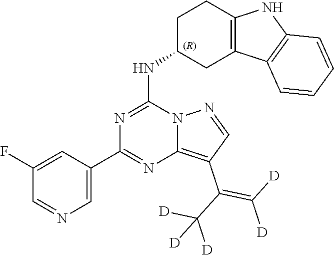 | I-70 |

In certain embodiments, the present invention provides any compound selected from those depicted in Table 1, above, or a pharmaceutically acceptable salt thereof

### 4. Uses, Formulation and Administration and Pharmaceutically Acceptable Compositions

According to another embodiment, the invention provides a composition comprising a compound of this invention or a pharmaceutically acceptable salt, ester, or salt of ester thereof and a pharmaceutically acceptable carrier, adjuvant, or vehicle. The amount of compound in compositions of this invention is such that is effective to measurably inhibit AHR, in a biological sample or in a patient. In certain embodiments, the amount of compound in compositions of this invention is such that is effective to measurably inhibit AHR, in a biological sample or in a patient. In certain embodiments, a composition of this invention is formulated for administration to a patient in need of such composition. In some embodiments, a composition of this invention is formulated for oral administration to a patient.

The term “patient,” as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this invention that, upon administration to a recipient, is capable of providing, either directly or indirectly, a compound of this invention or an inhibitorily active metabolite or residue thereof.

Compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

The amount of compounds of the present invention that may be combined with the carrier materials to produce a composition in a single dosage form will vary depending upon the host treated, the particular mode of administration. Preferably, provided compositions should be formulated so that a dosage of between 0.01-100 mg/kg body weight/day of the inhibitor can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

Uses of Compounds and Pharmaceutically Acceptable Compositions

The activity of a compound utilized in this invention as an inhibitor of AHR may be assayed in vitro or in vivo. An in vivo assessment of the efficacy of the compounds of the invention may be made using an animal model of obesity or metabolic syndrome, e.g., a rodent or primate model. Cell-based assays may be performed using, e.g., a cell line isolated from a tissue that expresses AHR. Additionally, biochemical or mechanism-based assays, e.g., transcription assays using a purified protein, Northern blot, RT-PCR, etc., may be performed. In vitro assays include assays that determine cell morphology, protein expression, and/or the cytotoxicity, enzyme inhibitory activity, and/or the subsequent functional consequences of treatment of cells with compounds of the invention. Alternate in vitro assays quantitate the ability of the inhibitor to bind to protein or nucleic acid molecules within the cell. Inhibitor binding may be measured by radiolabelling the inhibitor prior to binding, isolating the inhibitor/target molecule complex and determining the amount of radiolabel bound. Alternatively, inhibitor binding may be determined by running a competition experiment where new inhibitors are incubated with purified proteins or nucleic acids bound to known radioligands. Detailed conditions for assaying a compound utilized in this invention as an inhibitor of AHR are set forth in the Examples below. The aforementioned assays are exemplary and not intended to limit the scope of the invention. The skilled practitioner can appreciate that modifications can be made to conventional assays to develop equivalent assays that obtain the same result.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

The compounds and compositions, according to the method of the present invention, may be administered using any amount and any route of administration effective for treating or lessening the severity of a metabolic disorder or condition, cancer, a bacterial infection, a fungal infection, a parasitic infection (e.g. malaria), an autoimmune disorder, a neurodegenerative or neurological disorder, schizophrenia, a bone-related disorder, liver disease, or a cardiac disorder.

In some embodiments, the compounds and compositions, according to the method of the present invention, may be administered using any amount and any route of administration effective for treating or lessening the severity of a disease associated with AHR.

The exact amount required will vary from subject to subject, depending on the species, age, and general condition of the subject, the severity of the infection, the particular agent, its mode of administration, and the like. The compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts. The term “patient”, as used herein, means an animal, preferably a mammal, and most preferably a human.

The pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. In certain embodiments, the compounds of the invention may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

### Uses and Methods of Treatment

According to one embodiment, the invention relates to a method of inhibiting AHR in a biological sample comprising the step of contacting said biological sample with a compound of this invention, or a composition comprising said compound.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof.

Inhibition of enzymes in a biological sample is useful for a variety of purposes that are known to one of skill in the art. Examples of such purposes include, but are not limited to biological assays, gene expression studies, and biological target identification.

Another embodiment of the present invention relates to a method of inhibiting AHR in a patient comprising the step of administering to said patient a compound of the present invention, or a composition comprising said compound.

Provided compounds are inhibitors of AHR and are therefore useful for treating one or more disorders associated with activity of AHR. Thus, in certain embodiments, the present invention provides a method for treating an AHR-mediated disorder comprising the step of administering to a patient in need thereof a compound of the present invention, or pharmaceutically acceptable composition thereof.

As used herein, the terms “AHR-mediated” disorders, diseases, and/or conditions as used herein means any disease or other deleterious condition in which AHR, or a mutant thereof, are known to play a role. Accordingly, another embodiment of the present invention relates to treating or lessening the severity of one or more diseases in which AHR, or a mutant thereof, are known to play a role.

AHR mediated disorders are well established in the art. The nexus between AHR and AHR mediated disorders diseases and/or conditions as recited herein is well established in the relevant arts. For example, see: Uyttenhove et al., “Evidence for a tumoral immune resistance mechanism based on tryptophan degradation by indoleamine 2,3-dioxygenase” Nature Medicine, 2003 vol. 9(10), 1038; Murray et al., “AH RECEPTOR LIGANDS IN CANCER: FRIEND AND FOE” Nat. Rev. Cancer December 2014, vol. 14(12), pages 801-814; Moon et al., “Targeting the indoleamine 2,3-dioxygenase pathway in cancer” J. ImmunoTherapy of Cancer, 2015 vol 3, page 51; Ishida et al., “Activation of aryl hydrocarbon receptor promotes invasion of clear cell renal cell carcinoma and is associated with poor prognosis and cigarette smoke” Int. J Cancer July 2015 vol. 15, no. 137(2), pages 299-310; Ishida et al., “Activation of the aryl hydrocarbon receptor pathway enhances cancer cell invasion by upregulating the MMP expression and is associated with poor prognosis in upper urinary tract urothelial cancer” Carcinogenesis February 2010 vol. 31(2), pages 287-295. Su et al., “Prognostic value of nuclear translocation of aryl hydrocarbon receptor for non-small cell lung cancer” Anticancer Res. September 2013, vol. 33(9), pages 3953-3961; Peng et al., “Aryl hydrocarbon receptor pathway activation enhances gastric cancer cell invasiveness likely through a c-Jun-dependent induction of matrix metalloproteinase-9” BMC Cell Biol. April 2009 vol. 16; pages 10-27; Jin et al., “Aryl Hydrocarbon Receptor Activation Reduces Dendritic Cell Function during Influenza Virus Infection” Toxicol Sci. August 2010, vol. 116(2), pages 514-522; Head et al., “The aryl hydrocarbon receptor is a modulator of anti-viral immunity” Biochem. Pharmacol. February 2009 vol. 15; no. 77(4), pages 642-53; Jin et al., “New insights into the role of the aryl hydrocarbon receptor in the function of CD11c⁺ cells during respiratory viral infection” Eur. J. Immunol. June 2014, vol. 44(6), pages 1685-98; Nguyen et al., “Aryl hydrocarbon receptor and kynurenine: recent advances in autoimmune disease research” Front Immunol. October 2014, vol. 29, no. 5, page 551; Esser et al., “The aryl hydrocarbon receptor in immunity” Trends in Immunology, Vol. 30, No. 9.

In some embodiments, the present invention provides a method for treating one or more disorders, diseases, and/or conditions wherein the disorder, disease, or condition is a proliferative disease such as cancer, an inflammatory disorder, or a viral infection.

In certain embodiments, the present invention provides a method of treating cancer or another proliferative disorder, comprising administering a compound or composition of the present invention to a patient with cancer or another proliferative disorder. In certain embodiments, the method of treating cancer or another proliferative disorder comprises administering compounds and compositions of the present invention to a mammal. In certain embodiments, the mammal is a human.

As used herein, the terms “inhibition of cancer” and “inhibition of cancer cell proliferation” refer to the inhibition of the growth, division, maturation or viability of cancer cells, and/or causing the death of cancer cells, individually or in aggregate with other cancer cells, by cytotoxicity, nutrient depletion, or the induction of apoptosis.

Examples of tissues containing cancerous cells whose proliferation is inhibited by the compounds and compositions described herein and against which the methods described herein are useful include but are not limited to breast, prostate, brain, blood, bone marrow, liver, pancreas, skin, kidney, colon, ovary, lung, testicle, penis, thyroid, parathyroid, pituitary, thymus, retina, uvea, conjunctiva, spleen, head, neck, trachea, gall bladder, rectum, salivary gland, adrenal gland, throat, esophagus, lymph nodes, sweat glands, sebaceous glands, muscle, heart, and stomach.

In some embodiments, the cancer treated by compounds or compositions of the invention is a melanoma, liposarcoma, lung cancer, breast cancer, prostate cancer, leukemia, kidney cancer, esophageal cancer, brain cancer, lymphoma or colon cancer. In certain embodiments, the cancer is a primary effusion lymphoma (PEL).

Compounds of the current invention are useful in the treatment of a proliferative disease selected from a benign or malignant tumor, carcinoma of the brain, kidney, liver, adrenal gland, bladder, breast, stomach, gastric tumors, ovaries, colon, rectum, prostate, pancreas, lung, vagina, cervix, testis, genitourinary tract, esophagus, larynx, skin, bone or thyroid, sarcoma, glioblastomas, neuroblastomas, multiple myeloma or gastrointestinal cancer, especially colon carcinoma or colorectal adenoma or a tumor of the neck and head, an epidermal hyperproliferation, psoriasis, prostate hyperplasia, a neoplasia, a neoplasia of epithelial character, adenoma, adenocarcinoma, keratoacanthoma, epidermoid carcinoma, large cell carcinoma, non-small-cell lung carcinoma, lymphomas, Hodgkins and Non-Hodgkins, Waldenström's macroglobulinemia, a mammary carcinoma, follicular carcinoma, undifferentiated carcinoma, papillary carcinoma, seminoma, melanoma, an MYD88-driven disorder, DLBCL, ABC DLBCL, an IL-1-driven disorder, Smoldering of indolent multiple myeloma, or a leukemia.

Cancer includes, in some embodiments, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease or non-Hodgkin's disease), Waldenstrom's macroglobulinemia, multiple myeloma, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, glioblastoma multiforme (GBM, also known as glioblastoma), medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, and retinoblastoma).

In some embodiments, the cancer is glioma, astrocytoma, glioblastoma multiforme (GBM, also known as glioblastoma), medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, or retinoblastoma.

In some embodiments, the cancer is acoustic neuroma, astrocytoma (e.g. Grade I—Pilocytic Astrocytoma, Grade II—Low-grade Astrocytoma, Grade III—Anaplastic Astrocytoma, or Grade IV—Glioblastoma (GBM)), chordoma, CNS lymphoma, craniopharyngioma, brain stem glioma, ependymoma, mixed glioma, optic nerve glioma, subependymoma, medulloblastoma, meningioma, metastatic brain tumor, oligodendroglioma, pituitary tumors, primitive neuroectodermal (PNET) tumor, or schwannoma. In some embodiments, the cancer is a type found more commonly in children than adults, such as brain stem glioma, craniopharyngioma, ependymoma, juvenile pilocytic astrocytoma (JPA), medulloblastoma, optic nerve glioma, pineal tumor, primitive neuroectodermal tumors (PNET), or rhabdoid tumor. In some embodiments, the patient is an adult human. In some embodiments, the patient is a child or pediatric patient.

Cancer includes, in another embodiment, without limitation, mesothelioma, hepatobilliary (hepatic and billiary duct), bone cancer, pancreatic cancer, skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, non-Hodgkins's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers.

In some embodiments, the cancer is selected from hepatocellular carcinoma, ovarian cancer, ovarian epithelial cancer, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical adenoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; or medulloblastoma.

In some embodiments, the cancer is selected from hepatocellular carcinoma (HCC), hepatoblastoma, colon cancer, rectal cancer, ovarian cancer, ovarian epithelial cancer, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical adenoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In some embodiments, the cancer is a solid tumor, such as a sarcoma, carcinoma, or lymphoma. Solid tumors generally comprise an abnormal mass of tissue that typically does not include cysts or liquid areas. In some embodiments, the cancer is selected from renal cell carcinoma, or kidney cancer; hepatocellular carcinoma (HCC) or hepatoblastoma, or liver cancer; melanoma; breast cancer; colorectal carcinoma, or colorectal cancer; colon cancer; rectal cancer; anal cancer; lung cancer, such as non-small cell lung cancer (NSCLC) or small cell lung cancer (SCLC); ovarian cancer, ovarian epithelial cancer, ovarian carcinoma, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical carcinoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; or medulloblastoma.

In some embodiments, the cancer is selected from renal cell carcinoma, hepatocellular carcinoma (HCC), hepatoblastoma, colorectal carcinoma, colorectal cancer, colon cancer, rectal cancer, anal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, chondrosarcoma, anaplastic thyroid cancer, adrenocortical carcinoma, pancreatic cancer, pancreatic ductal carcinoma, pancreatic adenocarcinoma, glioma, brain cancer, neurofibromatosis-1 associated malignant peripheral nerve sheath tumors (MPNST), Waldenstrom's macroglobulinemia, or medulloblastoma.

In some embodiments, the cancer is selected from hepatocellular carcinoma (HCC), hepatoblastoma, colon cancer, rectal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical carcinoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In some embodiments, the cancer is hepatocellular carcinoma (HCC). In some embodiments, the cancer is hepatoblastoma. In some embodiments, the cancer is colon cancer. In some embodiments, the cancer is rectal cancer. In some embodiments, the cancer is ovarian cancer, or ovarian carcinoma. In some embodiments, the cancer is ovarian epithelial cancer. In some embodiments, the cancer is fallopian tube cancer. In some embodiments, the cancer is papillary serous cystadenocarcinoma. In some embodiments, the cancer is uterine papillary serous carcinoma (UPSC). In some embodiments, the cancer is hepatocholangiocarcinoma. In some embodiments, the cancer is soft tissue and bone synovial sarcoma. In some embodiments, the cancer is rhabdomyosarcoma. In some embodiments, the cancer is osteosarcoma. In some embodiments, the cancer is anaplastic thyroid cancer. In some embodiments, the cancer is adrenocortical carcinoma. In some embodiments, the cancer is pancreatic cancer, or pancreatic ductal carcinoma. In some embodiments, the cancer is pancreatic adenocarcinoma. In some embodiments, the cancer is glioma. In some embodiments, the cancer is malignant peripheral nerve sheath tumors (MPNST). In some embodiments, the cancer is neurofibromatosis-1 associated MPNST. In some embodiments, the cancer is Waldenstrom's macroglobulinemia. In some embodiments, the cancer is medulloblastoma.

In some embodiments, the cancer is Acute Lymphoblastic Leukemia (ALL), Acute Myeloid Leukemia (AML), Adrenocortical Carcinoma, Anal Cancer, Appendix Cancer, Atypical Teratoid/Rhabdoid Tumor, Basal Cell Carcinoma, Bile Duct Cancer, Bladder Cancer, Bone Cancer, Brain Tumor, Astrocytoma, Brain and Spinal Cord Tumor, Brain Stem Glioma, Central Nervous System Atypical Teratoid/Rhabdoid Tumor, Central Nervous System Embryonal Tumors, Breast Cancer, Bronchial Tumors, Burkitt Lymphoma, Carcinoid Tumor, Carcinoma of Unknown Primary, Central Nervous System Cancer, Cervical Cancer, Childhood Cancers, Chordoma, Chronic Lymphocytic Leukemia (CLL), Chronic Myelogenous Leukemia (CML), Chronic Myeloproliferative Disorders, Colon Cancer, Colorectal Cancer, Craniopharyngioma, Cutaneous T-Cell Lymphoma, Ductal Carcinoma In Situ (DCIS), Embryonal Tumors, Endometrial Cancer, Ependymoblastoma, Ependymoma, Esophageal Cancer, Esthesioneuroblastoma, Ewing Sarcoma, Extracranial Germ Cell Tumor, Extragonadal Germ Cell Tumor, Extrahepatic Bile Duct Cancer, Eye Cancer, Fibrous Histiocytoma of Bone, Gallbladder Cancer, Gastric Cancer, Gastrointestinal Carcinoid Tumor, Gastrointestinal Stromal Tumors (GIST), Germ Cell Tumor, Ovarian Germ Cell Tumor, Gestational Trophoblastic Tumor, Glioma, Hairy Cell Leukemia, Head and Neck Cancer, Heart Cancer, Hepatocellular Cancer, Histiocytosis, Langerhans Cell Cancer, Hodgkin Lymphoma, Hypopharyngeal Cancer, Intraocular Melanoma, Islet Cell Tumors, Kaposi Sarcoma, Kidney Cancer, Langerhans Cell Histiocytosis, Laryngeal Cancer, Leukemia, Lip and Oral Cavity Cancer, Liver Cancer, Lobular Carcinoma In Situ (LCIS), Lung Cancer, Lymphoma, AIDS-Related Lymphoma, Macroglobulinemia, Male Breast Cancer, Medulloblastoma, Medulloepithelioma, Melanoma, Merkel Cell Carcinoma, Malignant Mesothelioma, Metastatic Squamous Neck Cancer with Occult Primary, Midline Tract Carcinoma Involving NUT Gene, Mouth Cancer, Multiple Endocrine Neoplasia Syndrome, Multiple Myeloma/Plasma Cell Neoplasm, Mycosis Fungoides, Myelodysplastic Syndrome, Myelodysplastic/Myeloproliferative Neoplasm, Chronic Myelogenous Leukemia (CML), Acute Myeloid Leukemia (AML), Myeloma, Multiple Myeloma, Chronic Myeloproliferative Disorder, Nasal Cavity Cancer, Paranasal Sinus Cancer, Nasopharyngeal Cancer, Neuroblastoma, Non-Hodgkin Lymphoma, Non-Small Cell Lung Cancer, Oral Cancer, Oral Cavity Cancer, Lip Cancer, Oropharyngeal Cancer, Osteosarcoma, Ovarian Cancer, Pancreatic Cancer, Papillomatosis, Paraganglioma, Paranasal Sinus Cancer, Nasal Cavity Cancer, Parathyroid Cancer, Penile Cancer, Pharyngeal Cancer, Pheochromocytoma, Pineal Parenchymal Tumors of Intermediate Differentiation, Pineoblastoma, Pituitary Tumor, Plasma Cell Neoplasm, Pleuropulmonary Blastoma, Breast Cancer, Primary Central Nervous System (CNS) Lymphoma, Prostate Cancer, Rectal Cancer, Renal Cell Cancer, Clear cell renal cell carcinoma, Renal Pelvis Cancer, Ureter Cancer, Transitional Cell Cancer, Retinoblastoma, Rhabdomyosarcoma, Salivary Gland Cancer, Sarcoma, Sezary Syndrome, Skin Cancer, Small Cell Lung Cancer, Small Intestine Cancer, Soft Tissue Sarcoma, Squamous Cell Carcinoma, Squamous Neck Cancer with Occult Primary, Squamous Cell Carcinoma of the Head and Neck (HNSCC), Stomach Cancer, Supratentorial Primitive Neuroectodermal Tumors, T-Cell Lymphoma, Testicular Cancer, Throat Cancer, Thymoma, Thymic Carcinoma, Thyroid Cancer, Transitional Cell Cancer of the Renal Pelvis and Ureter, Triple Negative Breast Cancer (TNBC), Gestational Trophoblastic Tumor, Unknown Primary, Unusual Cancer of Childhood, Urethral Cancer, Uterine Cancer, Uterine Sarcoma, Waldenstrom Macroglobulinemia, or Wilms Tumor.

Compounds according to the invention are useful in the treatment of inflammatory or obstructive airways diseases, resulting, for example, in reduction of tissue damage, airways inflammation, bronchial hyperreactivity, remodeling or disease progression. Inflammatory or obstructive airways diseases to which the present invention is applicable include asthma of whatever type or genesis including both intrinsic (non-allergic) asthma and extrinsic (allergic) asthma, mild asthma, moderate asthma, severe asthma, bronchitic asthma, exercise-induced asthma, occupational asthma and asthma induced following bacterial infection. Treatment of asthma is also to be understood as embracing treatment of subjects, e.g. of less than 4 or 5 years of age, exhibiting wheezing symptoms and diagnosed or diagnosable as “wheezy infants”, an established patient category of major medical concern and now often identified as incipient or early-phase asthmatics.

Prophylactic efficacy in the treatment of asthma will be evidenced by reduced frequency or severity of symptomatic attack, e.g. of acute asthmatic or bronchoconstrictor attack, improvement in lung function or improved airways hyperreactivity. It may further be evidenced by reduced requirement for other, symptomatic therapy, such as therapy for or intended to restrict or abort symptomatic attack when it occurs, for example antiinflammatory or bronchodilatory. Prophylactic benefit in asthma may in particular be apparent in subjects prone to “morning dipping”. “Morning dipping” is a recognized asthmatic syndrome, common to a substantial percentage of asthmatics and characterised by asthma attack, e.g. between the hours of about 4 to 6 am, i.e. at a time normally substantially distant form any previously administered symptomatic asthma therapy.

Compounds of the current invention can be used for other inflammatory or obstructive airways diseases and conditions to which the present invention is applicable and include acute lung injury (ALI), adult/acute respiratory distress syndrome (ARDS), chronic obstructive pulmonary, airways or lung disease (COPD, COAD or COLD), including chronic bronchitis or dyspnea associated therewith, emphysema, as well as exacerbation of airways hyperreactivity consequent to other drug therapy, in particular other inhaled drug therapy. The invention is also applicable to the treatment of bronchitis of whatever type or genesis including, but not limited to, acute, arachidic, catarrhal, croupus, chronic or phthinoid bronchitis. Further inflammatory or obstructive airways diseases to which the present invention is applicable include pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis.

With regard to their anti-inflammatory activity, in particular in relation to inhibition of eosinophil activation, compounds of the invention are also useful in the treatment of eosinophil related disorders, e.g. eosinophilia, in particular eosinophil related disorders of the airways (e.g. involving morbid eosinophilic infiltration of pulmonary tissues) including hypereosinophilia as it effects the airways and/or lungs as well as, for example, eosinophil-related disorders of the airways consequential or concomitant to Loffler's syndrome, eosinophilic pneumonia, parasitic (in particular metazoan) infestation (including tropical eosinophilia), bronchopulmonary aspergillosis, polyarteritis nodosa (including Churg-Strauss syndrome), eosinophilic granuloma and eosinophil-related disorders affecting the airways occasioned by drug-reaction.

Compounds of the invention are also useful in the treatment of inflammatory or allergic conditions of the skin, for example psoriasis, contact dermatitis, atopic dermatitis, alopecia areata, erythema multiforma, dermatitis herpetiformis, scleroderma, vitiligo, hypersensitivity angiitis, urticaria, bullous pemphigoid, lupus erythematosus, systemic lupus erythematosus, pemphigus vulgaris, pemphigus foliaceus, paraneoplastic pemphigus, epidermolysis bullosa acquisita, acne vulgaris, and other inflammatory or allergic conditions of the skin.

Compounds of the invention may also be used for the treatment of other diseases or conditions, such as diseases or conditions having an inflammatory component, for example, treatment of diseases and conditions of the eye such as ocular allergy, conjunctivitis, keratoconjunctivitis sicca, and vernal conjunctivitis, diseases affecting the nose including allergic rhinitis, and inflammatory disease in which autoimmune reactions are implicated or having an autoimmune component or etiology, including autoimmune hematological disorders (e.g. hemolytic anemia, aplastic anemia, pure red cell anemia and idiopathic thrombocytopenia), systemic lupus erythematosus, rheumatoid arthritis, polychondritis, scleroderma, Wegener granulamatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), irritable bowel syndrome, celiac disease, periodontitis, hyaline membrane disease, kidney disease, glomerular disease, alcoholic liver disease, multiple sclerosis, endocrine opthalmopathy, Graves' disease, sarcoidosis, alveolitis, chronic hypersensitivity pneumonitis, multiple sclerosis, primary biliary cirrhosis, uveitis (anterior and posterior), Sjogren's syndrome, keratoconjunctivitis sicca and vernal keratoconjunctivitis, interstitial lung fibrosis, psoriatic arthritis, systemic juvenile idiopathic arthritis, cryopyrin-associated periodic syndrome, nephritis, vasculitis, diverticulitis, interstitial cystitis, glomerulonephritis (with and without nephrotic syndrome, e.g. including idiopathic nephrotic syndrome or minal change nephropathy), chronic granulomatous disease, endometriosis, leptospiriosis renal disease, glaucoma, retinal disease, ageing, headache, pain, complex regional pain syndrome, cardiac hypertrophy, musclewasting, catabolic disorders, obesity, fetal growth retardation, hyperchlolesterolemia, heart disease, chronic heart failure, mesothelioma, anhidrotic ecodermal dysplasia, Behcet's disease, incontinentia pigmenti, Paget's disease, pancreatitis, hereditary periodic fever syndrome, asthma (allergic and non-allergic, mild, moderate, severe, bronchitic, and exercise-induced), acute lung injury, acute respiratory distress syndrome, eosinophilia, hypersensitivities, anaphylaxis, nasal sinusitis, ocular allergy, silica induced diseases, COPD (reduction of damage, airways inflammation, bronchial hyperreactivity, remodeling or disease progression), pulmonary disease, cystic fibrosis, acid-induced lung injury, pulmonary hypertension, polyneuropathy, cataracts, muscle inflammation in conjunction with systemic sclerosis, dermatomyositis, polymyositis, inclusion body myositis, myasthenia gravis, thyroiditis, Addison's disease, lichen planus, Type 1 diabetes, or Type 2 diabetes.

In some embodiments the inflammatory disease which can be treated according to the methods of this invention is selected from acute and chronic gout, chronic gouty arthritis, psoriasis, psoriatic arthritis, rheumatoid arthritis, Juvenile rheumatoid arthritis, Systemic juvenile idiopathic arthritis (SJIA), Cryopyrin-Associated Periodic Syndromes (CAPS), or osteoarthritis.

In some embodiments, the inflammatory disease which can be treated according to the methods of this invention is selected from a TH17-mediated disease. In some embodiments, the TH17-mediated disease is selected from Systemic lupus erythematosus, Multiple sclerosis, inflammatory bowel disease including Crohn's or ulcerative colitis.

In some embodiments, the inflammatory disease which can be treated according to the methods of this invention is selected from Sjogren's syndrome allergic disorders, osteoarthritis. Conditions of the eye such as ocular allergy, conjunctivitis, keratoconjunctivitis sicca, and vernal conjunctivitis, diseases affecting the nose including allergic rhinitis.

In some embodiments, the inflammatory disease which can be treated according to the methods of this invention is selected from contact dermatitis, atopic dermatitis, alopecia areata, erythema multiforma, dermatitis herpetiformis, scleroderma, vitiligo, hypersensitivity angiitis, urticaria, bullous pemphigoid, pemphigus vulgaris, pemphigus foliaceus, paraneoplastic pemphigus, epidermolysis bullosa acquisita, and other inflammatory or allergic conditions of the skin.

In certain embodiments, a provided compound is useful for treating a viral infection, disease, or condition. In some embodiments, the present invention provides a method of treating a viral disease selected from retroviral diseases, such as, HIV-1, HIV-2, human T-cell leukemia virus-I (HTLV-I), HTLV-II, HTLV-III, simian immunodeficiency virus (SIV), lymphadenopathy-associated virus (LAV-2), simian T-lymphotrophic virus-I (STLV-I), STLV-II, STLV-III, simian B-lymphotrophic (SBL) virus, Gibbon ape leukemia virus (GALV), bovine leukemia virus (BLV), equine infectious anemia virus (EIAV), feline leukemia virus (FELV), murine leukemia virus (MuLV), avian leukosis virus (ALV); other virus infections such as hepadnaviridae (Hepatitis B); herpesviridae (Herpes simplex I, Herpes simplex II, Varicella-Zoster, Epstein-Barr virus and cytomegalovirus); parvoviridae (human parvovirus B-19); papovaviridae (human papilloma virus types 1 to 60, JC and BK viruses); pox viruses (variola major, variola minor, vaccinia, monkey pox, cowpox, paravaccinia or milker's node virus, parapox or ORF virus, molluscum contagiosum) and cancers, lymphomas and other leukemias.

### Combination Therapies

Depending upon the particular condition, or disease, to be treated, additional therapeutic agents, which are normally administered to treat that condition, may be administered in combination with compounds and compositions of this invention. As used herein, additional therapeutic agents that are normally administered to treat a particular disease, or condition, are known as “appropriate for the disease, or condition, being treated”.

In certain embodiments, a provided compound, or a composition thereof, is administered in combination with another anti-cancer, cytotoxin, or chemotherapeutic agent, to a patient in need thereof.

In certain embodiments, the anti-cancer or chemotherapeutic agents used in combination with compounds or compositions of the invention include, but are not limited to metformin, phenformin, buformin, imatinib, nilotinib, gefitinib, sunitinib, carfilzomib, salinosporamide A, retinoic acid, cisplatin, carboplatin, oxaliplatin, mechlorethamine, cyclophosphamide, chlorambucil, ifosfamide, azathioprine, mercaptopurine, doxifluridine, fluorouracil, gemcitabine, methotrexate, tioguanine, vincristine, vinblastine, vinorelbine, vindesine, podophyllotoxin, etoposide, teniposide, tafluposide, paclitaxel, docetaxel, irinotecan, topotecan, amsacrine, actinomycin, doxorubicin, daunorubicin, valrubicin, idarubicin, epirubicin, plicamycin, mitomycin, mitoxantrone, melphalan, busulfan, capecitabine, pemetrexed, epothilones, 13-cis-Retinoic Acid, 2-CdA, 2-Chlorodeoxyadenosine, 5-Azacitidine, 5-Fluorouracil, 5-FU, 6-Mercaptopurine, 6-MP, 6-TG, 6-Thioguanine, Abraxane, Accutane®, Actinomycin-D, Adriamycin®, Adrucil®, Afinitor®, Agrylin®, Ala-Cort®, Aldesleukin, Alemtuzumab, ALIMTA, Alitretinoin, Alkaban-AQ®, Alkeran®, All-transretinoic Acid, Alpha Interferon, Altretamine, Amethopterin, Amifostine, Aminoglutethimide, Anagrelide, Anandron®, Anastrozole, Arabinosylcytosine, Ara-C, Aranesp®, Aredia®, Arimidex®, Aromasin®, Arranon®, Arsenic Trioxide, Arzerra™, Asparaginase, ATRA, Avastin®, Azacitidine, BCG, BCNU, Bendamustine, Bevacizumab, Bexarotene, BEXXAR®, Bicalutamide, BiCNU, Blenoxane®, Bleomycin, Bortezomib, Busulfan, Busulfex®, C225, Calcium Leucovorin, Campath®, Camptosar®, Camptothecin-11, Capecitabine, Carac™, Carboplatin, Carmustine, Carmustine Wafer, Casodex®, CC-5013, CCI-779, CCNU, CDDP, CeeNU, Cerubidine®, Cetuximab, Chlorambucil, Citrovorum Factor, Cladribine, Cortisone, Cosmegen®, CPT-11, Cytadren®, Cytosar-U®, Cytoxan®, Dacarbazine, Dacogen, Dactinomycin, Darbepoetin Alfa, Dasatinib, Daunomycin, Daunorubicin Hydrochloride, Daunorubicin Liposomal, DaunoXome®, Decadron, Decitabine, Delta-Cortef®, Deltasone®, Denileukin, Diftitox, DepoCyt™, Dexamethasone, Dexamethasone Acetate, Dexamethasone Sodium Phosphate, Dexasone, Dexrazoxane, DHAD, DIC, Diodex, Docetaxel, Doxil®, Doxorubicin, Doxorubicin Liposomal, Droxia™, DTIC, DTIC-Dome®, Duralone®, Efudex®, Eligard™, Ellence™, Eloxatin™, Elspar®, Emcyt®, Epirubicin, Epoetin Alfa, Erbitux, Erlotinib, Erwinia L-asparaginase, Estramustine, Ethyol, Etopophos®, Etoposide, Etoposide Phosphate, Eulexin®, Everolimus, Evista®, Exemestane, Fareston®, Faslodex®, Femara®, Filgrastim, Floxuridine, Fludara®, Fludarabine, Fluoroplex®, Fluorouracil, Fluorouracil (cream), Fluoxymesterone, Flutamide, Folinic Acid, FUDR®, Fulvestrant, G-CSF, Gefitinib, Gemcitabine, Gemtuzumab, ozogamicin, Gemzar Gleevec™ Gliadel® Wafer, GM-CSF, Goserelin, Granulocyte—Colony Stimulating Factor, Granulocyte Macrophage Colony Stimulating Factor, Halotestin®, Herceptin®, Hexadrol, Hexalen®, Hexamethylmelamine, HMM, Hycamtin®, Hydrea®, Hydrocort Acetate®, Hydrocortisone, Hydrocortisone Sodium Phosphate, Hydrocortisone Sodium Succinate, Hydrocortone Phosphate, Hydroxyurea, Ibritumomab, Ibritumomab, Tiuxetan, Idamycin®, Idarubicin Ifex®, IFN-alpha, Ifosfamide, IL-11, IL-2, Imatinib mesylate, Imidazole Carboxamide, Interferon alfa, Interferon Alfa-2b (PEG Conjugate), Interleukin-2, Interleukin-11, Intron A® (interferon alfa-2b), Iressa®, Irinotecan, Isotretinoin, Ixabepilone, Ixempra™, Kidrolase®, Lanacort®, Lapatinib, L-asparaginase, LCR, Lenalidomide, Letrozole, Leucovorin, Leukeran, Leukine™, Leuprolide, Leurocristine, Leustatin™, Liposomal Ara-C, Liquid Pred®, Lomustine, L-PAM, L-Sarcolysin, Lupron®, Lupron Depot®, Matulane®, Maxidex, Mechlorethamine, Mechlorethamine Hydrochloride, Medralone®, Medrol®, Megace®, Megestrol, Megestrol Acetate, Melphalan, Mercaptopurine, Mesna, Mesnex™, Methotrexate, Methotrexate Sodium, Methylprednisolone, Meticorten®, Mitomycin, Mitomycin-C, Mitoxantrone, M-Prednisol®, MTC, MTX, Mustargen®, Mustine, Mutamycin®, Myleran®, Mylocel™, Mylotarg®, Navelbine®, Nelarabine, Neosar®, Neulasta™, Neumega®, Neupogen®, Nexavar®, Nilandron®, Nilotinib, Nilutamide, Nipent®, Nitrogen Mustard, Novaldex®, Novantrone®, Nplate, Octreotide, Octreotide acetate, Ofatumumab, Oncospar®, Oncovin®, Ontak®, Onxal™, Oprelvekin, Orapred®, Orasone®, Oxaliplatin, Paclitaxel, Paclitaxel Protein-bound, Pamidronate, Panitumumab, Panretin®, Paraplatin®, Pazopanib, Pediapred®, PEG Interferon, Pegaspargase, Pegfilgrastim, PEG-INTRON™, PEG-L-asparaginase, PEMETREXED, Pentostatin, Phenylalanine Mustard, Platinol®, Platinol-AQ®, Prednisolone, Prednisone, Prelone®, Procarbazine, PROCRIT®, Proleukin®, Prolifeprospan 20 with Carmustine Implant, Purinethol®, Raloxifene, Revlimid®, Rheumatrex®, Rituxan®, Rituximab, Roferon-A® (Interferon Alfa-2a), Romiplostim, Rubex®, Rubidomycin hydrochloride, Sandostatin®, Sandostatin LAR®, Sargramostim, Solu-Cortef®, Solu-Medrol®, Sorafenib, SPRYCEL™, STI-571, Streptozocin, SU11248, Sunitinib, Sutent®, Tamoxifen, Tarceva®, Targretin®, Tasigna®, Taxol®, Taxotere®, Temodar®, Temozolomide, Temsirolimus, Teniposide, TESPA, Thalidomide, Thalomid®, TheraCys®, Thioguanine, Thioguanine Tabloid®, Thiophosphoamide, Thioplex®, Thiotepa, TICE®, Toposar®, Topotecan, Toremifene, Torisel®, Tositumomab, Trastuzumab, Treanda®, Tretinoin, Trexall™ Trisenox®, TSPA, TYKERB®, VCR, Vectibix™, Velban®, Velcade®, VePesid®, Vesanoid®, Viadur™, Vidaza®, Vinblastine, Vinblastine Sulfate, Vincasar Pfs®, Vincristine, Vinorelbine, Vinorelbine tartrate, VLB, VM-26, Vorinostat, Votrient, VP-16, Vumon®, Xeloda®, Zanosar®, Zevalin™, Zinecard®, Zoladex®, Zoledronic acid, Zolinza, Zometa®, or combinations of any of the above.

In certain embodiments, an immuno-oncology agent can be administered with a compound as described herein for treatment of a proliferative disorder as described herein. As used herein, the term “an immuno-oncology agent” refers to an agent which is effective to enhance, stimulate, and/or up-regulate immune responses in a subject. In some embodiments, the administration of an immuno-oncology agent with a compound as described herein has a synergic effect in treating cancer.

In some embodiments, a compound as described herein is sequentially administered prior to administration of an immuno-oncology agent. In some embodiments, a compound as described herein is administered concurrently with an immuno-oncology agent. In some embodiments, a compound as described herein is sequentially administered after administration of an immuno-oncology agent.

In some embodiments, a compound as described herein may be co-formulated with an immuno-oncology agent.

An immuno-oncology agent can be, for example, a small molecule drug, an antibody, or a biologic or small molecule. Examples of biologic immuno-oncology agents include, but are not limited to, cancer vaccines, antibodies, and cytokines. In some embodiments, an antibody is a monoclonal antibody. In some embodiments, a monoclonal antibody is humanized or human.

In some embodiments, an immuno-oncology agent is (i) an agonist of a stimulatory (including a co-stimulatory) receptor or (ii) an antagonist of an inhibitory (including a co-inhibitory) signal on T cells, both of which result in amplifying antigen-specific T cell responses.

Certain of the stimulatory and inhibitory molecules are members of the immunoglobulin super family (IgSF). One important family of membrane-bound ligands that bind to co-stimulatory or co-inhibitory receptors is the B7 family, which includes B7-1, B7-2, B7-H1 (PD-L1), B7-DC (PD-L2), B7-H2 (ICOS-L), B7-H3, B7-H4, B7-H5 (VISTA), and B7-H6. Another family of membrane bound ligands that bind to co-stimulatory or co-inhibitory receptors is the TNF family of molecules that bind to cognate TNF receptor family members, which includes CD40 and CD40L, OX-40, OX-40L, CD70, CD27L, CD30, CD30L, 4-1BBL, CD137 (4-1BB), TRAIL/Apo2-L, TRAILR1/DR4, TRAILR2/DR5, TRAILR3, TRAILR4, OPG, RANK, RANKL, TWEAKR/Fn14, TWEAK, BAFFR, EDAR, XEDAR, TACI, APRIL, BCMA, LTOR, LIGHT, DcR3, HVEM, VEGI/TL1A, TRAMP/DR3, EDAR, EDA1, XEDAR, EDA2, TNFR1, Lymphotoxin α/TNFβ, TNFR2, TNFα, LTOR, Lymphotoxin α1β2, FAS, FASL, RELT, DR6, TROY, NGFR.

In some embodiments, an immuno-oncology agent is a cytokine that inhibits T cell activation (e.g., IL-6, IL-10, TGF-β, VEGF, and other immunosuppressive cytokines) or a cytokine that stimulates T cell activation, for stimulating an immune response.

In some embodiments, a combination of a compound as described herein, and an immuno-oncology agent can stimulate T cell responses. In some embodiments, an immuno-oncology agent is: (i) an antagonist of a protein that inhibits T cell activation (e.g., immune checkpoint inhibitors) such as CTLA-4, PD-1, PD-L1, PD-L2, LAG-3, TIM-3, Galectin 9, CEACAM-1, BTLA, CD69, Galectin-1, TIGIT, CD113, GPR56, VISTA, 2B4, CD48, GARP, PD1H, LAIR1, TIM-1, and TIM-4; or (ii) an agonist of a protein that stimulates T cell activation such as B7-1, B7-2, CD28, 4-1BB (CD137), 4-1BBL, ICOS, ICOS-L, OX40, OX40L, GITR, GITRL, CD70, CD27, CD40, DR3 and CD28H.

In some embodiments, an immuno-oncology agent is an antagonist of inhibitory receptors on NK cells or an agonists of activating receptors on NK cells. In some embodiments, an immuno-oncology agent is an antagonists of KIR, such as lirilumab.

In some embodiments, an immuno-oncology agent is an agent that inhibits or depletes macrophages or monocytes, including but not limited to CSF-1R antagonists such as CSF-1R antagonist antibodies including RG7155 (WO11/70024, WO11/107553, WO11/131407, WO13/87699, WO13/119716, WO13/132044) or FPA-008 (WO11/140249; WO13169264; WO14/036357).

In some embodiments, an immuno-oncology agent is selected from agonistic agents that ligate positive costimulatory receptors, blocking agents that attenuate signaling through inhibitory receptors, antagonists, and one or more agents that increase systemically the frequency of anti-tumor T cells, agents that overcome distinct immune suppressive pathways within the tumor microenvironment (e.g., block inhibitory receptor engagement (e.g., PD-L1/PD-1 interactions), deplete or inhibit Tregs (e.g., using an anti-CD25 monoclonal antibody (e.g., daclizumab) or by ex vivo anti-CD25 bead depletion), inhibit metabolic enzymes such as IDO, or reverse/prevent T cell energy or exhaustion) and agents that trigger innate immune activation and/or inflammation at tumor sites.

In some embodiments, an immuno-oncology agent is a CTLA-4 antagonist. In some embodiments, a CTLA-4 antagonist is an antagonistic CTLA-4 antibody. In some embodiments, an antagonistic CTLA-4 antibody is YERVOY (ipilimumab) or tremelimumab.

In some embodiments, an immuno-oncology agent is a PD-1 antagonist. In some embodiments, a PD-1 antagonist is administered by infusion. In some embodiments, an immuno-oncology agent is an antibody or an antigen-binding portion thereof that binds specifically to a Programmed Death-1 (PD-1) receptor and inhibits PD-1 activity. In some embodiments, a PD-1 antagonist is an antagonistic PD-1 antibody. In some embodiments, an antagonistic PD-1 antibody is OPDIVO (nivolumab), KEYTRUDA (pembrolizumab), or MEDI-0680 (AMP-514; WO2012/145493). In some embodiments, an immuno-oncology agent may be pidilizumab (CT-011). In some embodiments, an immuno-oncology agent is a recombinant protein composed of the extracellular domain of PD-L2 (B7-DC) fused to the Fc portion of IgG1, called AMP-224.

In some embodiments, an immuno-oncology agent is a PD-L1 antagonist. In some embodiments, a PD-L1 antagonist is an antagonistic PD-L1 antibody. In some embodiments, a PD-L1 antibody is MPDL3280A (RG7446; WO2010/077634), durvalumab (MEDI4736), BMS-936559 (WO2007/005874), and MSB0010718C (WO2013/79174).

In some embodiments, an immuno-oncology agent is a LAG-3 antagonist. In some embodiments, a LAG-3 antagonist is an antagonistic LAG-3 antibody. In some embodiments, a LAG3 antibody is BMS-986016 (WO10/19570, WO14/08218), or IMP-731 or IMP-321 (WO08/132601, WO009/44273).

In some embodiments, an immuno-oncology agent is a CD137 (4-1BB) agonist. In some embodiments, a CD137 (4-1BB) agonist is an agonistic CD137 antibody. In some embodiments, a CD137 antibody is urelumab or PF-05082566 (WO12/32433).

In some embodiments, an immuno-oncology agent is a GITR agonist. In some embodiments, a GITR agonist is an agonistic GITR antibody. In some embodiments, a GITR antibody is BMS-986153, BMS-986156, TRX-518 (WO006/105021, WO009/009116), or MK-4166 (WO11/028683).

In some embodiments, an immuno-oncology agent is an IDO antagonist. In some embodiments, an IDO antagonist is INCB-024360 (WO2006/122150, WO07/75598, WO08/36653, WO08/36642), indoximod, or NLG-919 (WO09/73620, WO009/1156652, WO11/56652, WO12/142237).

In some embodiments, an immuno-oncology agent is an OX40 agonist. In some embodiments, an OX40 agonist is an agonistic OX40 antibody. In some embodiments, an OX40 antibody is MEDI-6383 or MEDI-6469.

In some embodiments, an immuno-oncology agent is an OX40L antagonist. In some embodiments, an OX40L antagonist is an antagonistic OX40 antibody. In some embodiments, an OX40L antagonist is RG-7888 (WO06/029879).

In some embodiments, an immuno-oncology agent is a CD40 agonist. In some embodiments, a CD40 agonist is an agonistic CD40 antibody. In some embodiments, an immuno-oncology agent is a CD40 antagonist. In some embodiments, a CD40 antagonist is an antagonistic CD40 antibody. In some embodiments, a CD40 antibody is lucatumumab or dacetuzumab.

In some embodiments, an immuno-oncology agent is a CD27 agonist. In some embodiments, a CD27 agonist is an agonistic CD27 antibody. In some embodiments, a CD27 antibody is varlilumab.

In some embodiments, an immuno-oncology agent is MGA271 (to B7H3) (WO11/109400).

In some embodiments, an immuno-oncology agent is abagovomab, adecatumumab, afutuzumab, alemtuzumab, anatumomab mafenatox, apolizumab, atezolimab, avelumab, blinatumomab, BMS-936559, catumaxomab, durvalumab, epacadostat, epratuzumab, indoximod, inotuzumab ozogamicin, intelumumab, ipilimumab, isatuximab, lambrolizumab, MED14736, MPDL3280A, nivolumab, obinutuzumab, ocaratuzumab, ofatumumab, olatatumab, pembrolizumab, pidilizumab, rituximab, ticilimumab, samalizumab, or tremelimumab.

In some embodiments, an immuno-oncology agent is an immunostimulatory agent. For example, antibodies blocking the PD-1 and PD-L1 inhibitory axis can unleash activated tumor-reactive T cells and have been shown in clinical trials to induce durable anti-tumor responses in increasing numbers of tumor histologies, including some tumor types that conventionally have not been considered immunotherapy sensitive. See, e.g., Okazaki, T. et al. (2013) Nat. Immunol. 14, 1212-1218; Zou et al. (2016) Sci. Transl. Med. 8. The anti-PD-1 antibody nivolumab (Opdivo®, Bristol-Myers Squibb, also known as ONO-4538, MDX1106 and BMS-936558), has shown potential to improve the overall survival in patients with RCC who had experienced disease progression during or after prior anti-angiogenic therapy.

In some embodiments, the immunomodulatory therapeutic specifically induces apoptosis of tumor cells. Approved immunomodulatory therapeutics which may be used in the present invention include pomalidomide (Pomalyst®, Celgene); lenalidomide (Revlimid®, Celgene); ingenol mebutate (Picato®, LEO Pharma).

In some embodiments, an immuno-oncology agent is a cancer vaccine. In some embodiments, the cancer vaccine is selected from sipuleucel-T (Provenge®, Dendreon/Valeant Pharmaceuticals), which has been approved for treatment of asymptomatic, or minimally symptomatic metastatic castrate-resistant (hormone-refractory) prostate cancer; and talimogene laherparepvec (Imlygic®, BioVex/Amgen, previously known as T-VEC), a genetically modified oncolytic viral therapy approved for treatment of unresectable cutaneous, subcutaneous and nodal lesions in melanoma. In some embodiments, an immuno-oncology agent is selected from an oncolytic viral therapy such as pexastimogene devacirepvec (PexaVec/JX-594, SillaJen/formerly Jennerex Biotherapeutics), a thymidine kinase- (TK-) deficient vaccinia virus engineered to express GM-CSF, for hepatocellular carcinoma (NCT02562755) and melanoma (NCT00429312); pelareorep (Reolysin®, Oncolytics Biotech), a variant of respiratory enteric orphan virus (reovirus) which does not replicate in cells that are not RAS-activated, in numerous cancers, including colorectal cancer (NCT01622543); prostate cancer (NCT01619813); head and neck squamous cell cancer (NCT01166542); pancreatic adenocarcinoma (NCT00998322); and non-small cell lung cancer (NSCLC) (NCT 00861627); enadenotucirev (NG-348, PsiOxus, formerly known as ColoAd1), an adenovirus engineered to express a full length CD80 and an antibody fragment specific for the T-cell receptor CD3 protein, in ovarian cancer (NCT02028117); metastatic or advanced epithelial tumors such as in colorectal cancer, bladder cancer, head and neck squamous cell carcinoma and salivary gland cancer (NCT02636036); ONCOS-102 (Targovax/formerly Oncos), an adenovirus engineered to express GM-CSF, in melanoma (NCT03003676); and peritoneal disease, colorectal cancer or ovarian cancer (NCT02963831); GL-ONC1 (GLV-1h68/GLV-1h153, Genelux GmbH), vaccinia viruses engineered to express beta-galactosidase (beta-gal)/beta-glucoronidase or beta-gal/human sodium iodide symporter (hNIS), respectively, were studied in peritoneal carcinomatosis (NCT01443260); fallopian tube cancer, ovarian cancer (NCT 02759588); or CG0070 (Cold Genesys), an adenovirus engineered to express GM-CSF, in bladder cancer (NCT02365818).

In some embodiments, an immuno-oncology agent is selected from JX-929 (SillaJen/formerly Jennerex Biotherapeutics), a TK- and vaccinia growth factor-deficient vaccinia virus engineered to express cytosine deaminase, which is able to convert the prodrug 5-fluorocytosine to the cytotoxic drug 5-fluorouracil; TG01 and TG02 (Targovax/formerly Oncos), peptide-based immunotherapy agents targeted for difficult-to-treat RAS mutations; and TILT-123 (TILT Biotherapeutics), an engineered adenovirus designated: Ad5/3-E2F-delta24-hTNFα-IRES-hIL20; and VSV-GP (ViraTherapeutics) a vesicular stomatitis virus (VSV) engineered to express the glycoprotein (GP) of lymphocytic choriomeningitis virus (LCMV), which can be further engineered to express antigens designed to raise an antigen-specific CD8⁺ T cell response.

In some embodiments, an immuno-oncology agent is a T-cell engineered to express a chimeric antigen receptor, or CAR. The T-cells engineered to express such chimeric antigen receptor are referred to as a CAR-T cells.

CARs have been constructed that consist of binding domains, which may be derived from natural ligands, single chain variable fragments (scFv) derived from monoclonal antibodies specific for cell-surface antigens, fused to endodomains that are the functional end of the T-cell receptor (TCR), such as the CD3-zeta signaling domain from TCRs, which is capable of generating an activation signal in T lymphocytes. Upon antigen binding, such CARs link to endogenous signaling pathways in the effector cell and generate activating signals similar to those initiated by the TCR complex.

For example, in some embodiments the CAR-T cell is one of those described in U.S. Pat. No. 8,906,682 (June; hereby incorporated by reference in its entirety), which discloses CAR-T cells engineered to comprise an extracellular domain having an antigen binding domain (such as a domain that binds to CD19), fused to an intracellular signaling domain of the T cell antigen receptor complex zeta chain (such as CD3 zeta). When expressed in the T cell, the CAR is able to redirect antigen recognition based on the antigen binding specificity. In the case of CD19, the antigen is expressed on malignant B cells. Over 200 clinical trials are currently in progress employing CAR-T in a wide range of indications. [https://clinicaltrials.gov/ct2/results?term=chimeric+antigen+receptors&pg=1].

In some embodiments, an immunostimulatory agent is an activator of retinoic acid receptor-related orphan receptor γ (RORγt). RORγt is a transcription factor with key roles in the differentiation and maintenance of Type 17 effector subsets of CD4+(Th17) and CD8+(Tc17) T cells, as well as the differentiation of IL-17 expressing innate immune cell subpopulations such as NK cells. In some embodiments, an activator of RORγt is LYC-55716 (Lycera), which is currently being evaluated in clinical trials for the treatment of solid tumors (NCT02929862).

In some embodiments, an immunostimulatory agent is an agonist or activator of a toll-like receptor (TLR). Suitable activators of TLRs include an agonist or activator of TLR9 such as SD-101 (Dynavax). SD-101 is an immunostimulatory CpG which is being studied for B-cell, follicular and other lymphomas (NCT02254772). Agonists or activators of TLR8 which may be used in the present invention include motolimod (VTX-2337, VentiRx Pharmaceuticals) which is being studied for squamous cell cancer of the head and neck (NCT02124850) and ovarian cancer (NCT02431559).

Other immuno-oncology agents that may be used in the present invention include urelumab (BMS-663513, Bristol-Myers Squibb), an anti-CD137 monoclonal antibody; varlilumab (CDX-1127, Celldex Therapeutics), an anti-CD27 monoclonal antibody; BMS-986178 (Bristol-Myers Squibb), an anti-OX40 monoclonal antibody; lirilumab (IPH2102/BMS-986015, Innate Pharma, Bristol-Myers Squibb), an anti-KIR monoclonal antibody; monalizumab (IPH2201, Innate Pharma, AstraZeneca) an anti-NKG2A monoclonal antibody; andecaliximab (GS-5745, Gilead Sciences), an anti-MMP9 antibody; MK-4166 (Merck & Co.), an anti-GITR monoclonal antibody.

In some embodiments, an immunostimulatory agent is selected from elotuzumab, mifamurtide, an agonist or activator of a toll-like receptor, and an activator of RORγt.

In some embodiments, an immunostimulatory therapeutic is recombinant human interleukin 15 (rhIL-15). rhIL-15 has been tested in the clinic as a therapy for melanoma and renal cell carcinoma (NCT01021059 and NCT01369888) and leukemias (NCT02689453). In some embodiments, an immunostimulatory agent is recombinant human interleukin 12 (rhIL-12). In some embodiments, an IL-15 based immunotherapeutic is heterodimeric IL-15 (hetIL-15, Novartis/Admune), a fusion complex composed of a synthetic form of endogenous IL-15 complexed to the soluble IL-15 binding protein IL-15 receptor alpha chain (IL15:sIL-15RA), which has been tested in Phase 1 clinical trials for melanoma, renal cell carcinoma, non-small cell lung cancer and head and neck squamous cell carcinoma (NCT02452268). In some embodiments, a recombinant human interleukin 12 (rhIL-12) is NM-IL-12 (Neumedicines, Inc.), NCT02544724, or NCT02542124.

In some embodiments, an immuno-oncology agent is selected from those descripted in Jerry L. Adams ET. AL., “Big opportunities for small molecules in immuno-oncology,” Cancer Therapy 2015, Vol. 14, pages 603-622, the content of which is incorporated herein by reference in its entirety. In some embodiments, an immuno-oncology agent is selected from the examples described in Table 1 of Jerry L. Adams ET. AL. In some embodiments, an immuno-oncology agent is a small molecule targeting an immuno-oncoloby target selected from those listed in Table 2 of Jerry L. Adams ET. AL. In some embodiments, an immuno-oncology agent is a small molecule agent selected from those listed in Table 2 of Jerry L. Adams ET. AL.

In some embodiments, an immuno-oncology agent is selected from the small molecule immuno-oncology agents described in Peter L. Toogood, “Small molecule immuno-oncology therapeutic agents,” Bioorganic & Medicinal Chemistry Letters 2018, Vol. 28, pages 319-329, the content of which is incorporated herein by reference in its entirety. In some embodiments, an immuno-oncology agent is an agent targeting the pathways as described in Peter L. Toogood.

In some embodiments, an immuno-oncology agent is selected from those described in Sandra L. Ross et al., “Bispecific T cell engager (BiTE®) antibody constructs can mediate bystander tumor cell killing”, PLoS ONE 12(8): e0183390, the content of which is incorporated herein by reference in its entirety. In some embodiments, an immuno-oncology agent is a bispecific T cell engager (BiTE®) antibody construct. In some embodiments, a bispecific T cell engager (BiTE®) antibody construct is a CD19/CD3 bispecific antibody construct. In some embodiments, a bispecific T cell engager (BiTE®) antibody construct is an EGFR/CD3 bispecific antibody construct. In some embodiments, a bispecific T cell engager (BiTE®) antibody construct activates T cells. In some embodiments, a bispecific T cell engager (BiTE®) antibody construct activates T cells, which release cytokines inducing upregulation of intercellular adhesion molecule 1 (ICAM-1) and FAS on bystander cells. In some embodiments, a bispecific T cell engager (BiTE®) antibody construct activates T cells which result in induced bystander cell lysis. In some embodiments, the bystander cells are in solid tumors. In some embodiments, the bystander cells being lysed are in proximity to the BiTE®-activated T cells. In some embodiment, the bystander cells comprises tumor-associated antigen (TAA) negative cancer cells. In some embodiment, the bystander cells comprise EGFR-negative cancer cells. In some embodiments, an immuno-oncology agent is an antibody which blocks the PD-L1/PD1 axis and/or CTLA4. In some embodiments, an immuno-oncology agent is an ex-vivo expanded tumor-infiltrating T cell. In some embodiments, an immuno-oncology agent is a bispecific antibody construct or chimeric antigen receptors (CARs) that directly connect T cells with tumor-associated surface antigens (TAAs).

In certain embodiments, a combination of 2 or more therapeutic agents may be administered together with compounds of the invention. In certain embodiments, a combination of 3 or more therapeutic agents may be administered with compounds of the invention.

Other examples of agents the inhibitors of this invention may also be combined with include, without limitation: vitamins and nutritional supplements, cancer vaccines, treatments for neutropenia (e.g. G-CSF, filgrastim, lenograstim), treatments for thrombocytopenia (e.g. blood transfusion, erythropoietin), PI3 kinase (PI3K) inhibitors, MEK inhibitors, mTOR inhibitors, CPT1 inhibitors, AMPK activators, PCSK9 inhibitors, SREBP site 1 protease inhibitors, HMG CoA-reductase inhibitors, antiemetics (e.g. 5-HT3 receptor antagonists, dopamine antagonists, NK1 receptor antagonists, histamine receptor antagonists, cannabinoids, benzodiazepines, or anticholinergics), treatments for Alzheimer's Disease such as Aricept® and Excelon®; treatments for Parkinson's Disease such as L-DOPA/carbidopa, entacapone, ropinrole, pramipexole, bromocriptine, pergolide, trihexephendyl, and amantadine; agents for treating Multiple Sclerosis (MS) such as beta interferon (e.g., Avonex® and Rebif®), Copaxone®, and mitoxantrone; treatments for asthma such as albuterol and Singulair®; agents for treating schizophrenia such as zyprexa, risperdal, seroquel, and haloperidol; anti-inflammatory agents such as corticosteroids, TNF blockers, IL-1 RA, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophophamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins, fibrates, cholesterol absorption inhibitors, bile acid sequestrants, and niacin; agents for treating liver disease such as corticosteroids, cholestyramine, interferons, and anti-viral agents; agents for treating blood disorders such as corticosteroids, anti-leukemic agents, and growth factors; agents for treating immunodeficiency disorders such as gamma globulin; and anti-diabetic agents such as biguanides (metformin, phenformin, buformin), thiazolidinediones (rosiglitazone, pioglitazone, troglitazone), sulfonylureas (tolbutamide, acetohexamide, tolazamide, chlorpropamide, glipizide, glyburide, glimepiride, gliclazide), meglitinides (repaglinide, nateglinide), alpha-glucosidase inhibitors (miglitol, acarbose), incretin mimetics (exenatide, liraglutide, taspoglutide), gastric inhibitory peptide analogs, DPP-4 inhibitors (vildagliptin, sitagliptin, saxagliptin, linagliptin, alogliptin), amylin analogs (pramlintide), and insulin and insulin analogs.

In certain embodiments, compounds of the present invention, or a pharmaceutically acceptable composition thereof, are administered in combination with antisense agents, a monoclonal or polyclonal antibody or an siRNA therapeutic.

In another embodiment, the present invention provides a method of treating an inflammatory disease, disorder or condition by administering to a patient in need thereof a compound of the present invention and one or more additional therapeutic agents. Such additional therapeutic agents may be small molecules or recombinant biologic agents and include, for example, acetaminophen, non-steroidal anti-inflammatory drugs (NSAIDS) such as aspirin, ibuprofen, naproxen, etodolac (Lodine®) and celecoxib, colchicine (Colcrys®), corticosteroids such as prednisone, prednisolone, methylprednisolone, hydrocortisone, and the like, probenecid, allopurinol, febuxostat (Uloric®), sulfasalazine (Azulfidine®), antimalarials such as hydroxychloroquine (Plaquenil®) and chloroquine (Aralen®), methotrexate (Rheumatrex®), gold salts such as gold thioglucose (Solganal®), gold thiomalate (Myochrysine®) and auranofin (Ridaura®), D-penicillamine (Depen® or Cuprimine®), azathioprine (Imuran®), cyclophosphamide (Cytoxan®), chlorambucil (Leukeran®), cyclosporine (Sandimmune®), leflunomide (Arava®) and “anti-TNF” agents such as etanercept (Enbrel®), infliximab (Remicade®), golimumab (Simponi®), certolizumab pegol (Cimzia®) and adalimumab (Humira®), “anti-IL-1” agents such as anakinra (Kineret®) and rilonacept (Arcalyst®), canakinumab (Ilaris®), anti-Jak inhibitors such as tofacitinib, antibodies such as rituximab (Rituxan®), “anti-T-cell” agents such as abatacept (Orencia®), “anti-IL-6” agents such as tocilizumab (Actemra®), diclofenac, cortisone, hyaluronic acid (Synvisc® or Hyalgan®), monoclonal antibodies such as tanezumab, anticoagulants such as heparin (Calcinparine® or Liquaemin®) and warfarin (Coumadin®), antidiarrheals such as diphenoxylate (Lomotil®) and loperamide (Imodium®), bile acid binding agents such as cholestyramine, alosetron (Lotronex®), lubiprostone (Amitiza®), laxatives such as Milk of Magnesia, polyethylene glycol (MiraLax®), Dulcolax®, Correctol® and Senokot®, anticholinergics or antispasmodics such as dicyclomine (Bentyl®), Singulair®, beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), inhaled corticosteroids such as beclomethasone dipropionate (Beclovent®, Qvar®, and Vanceril®), triamcinolone acetonide (Azmacort®), mometasone (Asthmanex®), budesonide (Pulmocort®), and flunisolide (Aerobid®), Afviar®, Symbicort®, Dulera®, cromolyn sodium (Intal®), methylxanthines such as theophylline (Theo-Dur®, Theolair®, Slo-bid®, Uniphyl®, Theo-24®) and aminophylline, IgE antibodies such as omalizumab (Xolair®), nucleoside reverse transcriptase inhibitors such as zidovudine (Retrovir®), abacavir (Ziagen®), abacavir/lamivudine (Epzicom®), abacavir/lamivudine/zidovudine (Trizivir®), didanosine (Videx®), emtricitabine (Emtriva®), lamivudine (Epivir®), lamivudine/zidovudine (Combivir®), stavudine (Zerit®), and zalcitabine (Hivid®), non-nucleoside reverse transcriptase inhibitors such as delavirdine (Rescriptor®), efavirenz (Sustiva®), nevairapine (Viramune®) and etravirine (Intelence®), nucleotide reverse transcriptase inhibitors such as tenofovir (Viread®), protease inhibitors such as amprenavir (Agenerase®), atazanavir (Reyataz®), darunavir (Prezista®), fosamprenavir (Lexiva®), indinavir (Crixivan®), lopinavir and ritonavir (Kaletra®), nelfinavir (Viracept®), ritonavir (Norvir®), saquinavir (Fortovase® or Invirase®), and tipranavir (Aptivus®), entry inhibitors such as enfuvirtide (Fuzeon®) and maraviroc (Selzentry®), integrase inhibitors such as raltegravir (Isentress®), doxorubicin (Hydrodaunorubicin®), vincristine (Oncovin®), bortezomib (Velcade®), and dexamethasone (Decadron®) in combination with lenalidomide (Revlimid®), or any combination(s) thereof.

In some embodiments, a provided compound is administered in combination with an antiviral agent, including, e.g., acyclovir, pencyclovir, cidofovir, idoxuridine, zidovudine, ribavarin, amantadine, foscarnet, didanosine, acyclovir, ganciclovir, cidofovir, zalcitabine, rimantadine, calacyclovir, famiciclovir, abacavir, didanosine, emtricitabine, lamivudine, stavudine, tenofovir, zalcitabine, zidovudine, zidovudine-lamivudine, TRIZIVIR (zidovudine, lamivudine, abacavir), EPZICOM (aba-cavir-lamivudine), TRUVADA (tenofovir-emtricitabine), efavirenz, nevirapine, and delavirdine, amprenavir, atazanavir, fosamprenavir, indinavir, lopinavir-ritonavir, nelfinavir, ritonavir, saquinavir, and tipranavir. In some embodiments, the antiviral agent is anti-influenza agent including, e.g., rimantadine, amantadine, oseltamivir, and zanamivir.

Those additional agents may be administered separately from an inventive compound-containing composition, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another, normally within five hours from one another.

As used herein, the term “combination,” “combined,” and related terms refers to the simultaneous or sequential administration of therapeutic agents in accordance with this invention. For example, a compound of the present invention may be administered with another therapeutic agent simultaneously or sequentially in separate unit dosage forms or together in a single unit dosage form. Accordingly, the present invention provides a single unit dosage form comprising a compound of the present invention, an additional therapeutic agent, and a pharmaceutically acceptable carrier, adjuvant, or vehicle.

The amount of both, a provided compound and additional therapeutic agent (in those compositions which comprise an additional therapeutic agent as described above) that may be combined with the carrier materials to produce a single dosage form will vary depending upon the host treated and the particular mode of administration. Preferably, compositions of this invention should be formulated so that a dosage of between 0.01-100 mg/kg body weight/day of an inventive can be administered.

In those compositions which comprise an additional therapeutic agent, that additional therapeutic agent and the compound of this invention may act synergistically. Therefore, the amount of additional therapeutic agent in such compositions will be less than that required in a monotherapy utilizing only that therapeutic agent. In such compositions a dosage of between 0.01-100 μg/kg body weight/day of the additional therapeutic agent can be administered.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

In one embodiment, the present invention provides a composition comprising a compound of the present invention and one or more additional therapeutic agents. The therapeutic agent may be administered together with a compound of the present invention, or may be administered prior to or following administration of a compound of the present invention. Suitable therapeutic agents are described in further detail below. In certain embodiments, a compound of the present invention may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours before the therapeutic agent. In other embodiments, a compound of formula I may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours following the therapeutic agent.

In some embodiments, the present invention provides a medicament comprising at least one compound of the present invention or a pharmaceutically acceptable salt thereof and a pharmaceutically acceptable carrier.

### EXEMPLIFICATION

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein.

### Example 1A

### DRE-Luciferase Reporter Assay

AHR binds to Dioxin Responsive Elements (DRE) upstream of genes that it activates. One measure of AHR activity is activation of a reporter gene, such as luciferase, downstream of one or multiple DRE elements. Luciferase activity will reflect activation and inhibition of AHR in the cells expressing his reporter.

Murine Hepa1-6 or Hepa-1c1c7 or other murine cell line with a DRE-luciferase reporter either stably or transiently transfected were plated in media in plates (96-well, 384-well or other plates) and incubated overnight at 37° C. in a CO₂ incubator. Likewise, human HepG2 or other human cell line with a DRE-luciferase reporter either stably or transiently transfected were plated in media in plates (96-well, 384-well or other plates) and incubated overnight at 37° C. in a CO₂ incubator.

The next day, an AHR activating ligand, such as TCDD, kynurenine, ITE (2-(1H-indole-3-ylcarbonyl)-4-thiazolecarboxylic methyl ester), VAF347, BNF (beta-naphthoflavone), FICZ (6-formylindolo(3,2-b) carbazole or other AHR ligands, was added with or without AHR antagonist.

Cells were incubated for 4, 15 or 24 hours or another time point and then lysed for determination of luciferase activity as a read-out of the AHR activation or inhibition. Luciferase was measured with a commercial kit such as the Promega Luciferase kit or any kit or reagents that provide the luciferin substrate for measuring luciferase activity. The level of luciferase with only activating ligand added was the maximum signal while the luciferase with no ligand was the minimum signal. IC₅₀ values were determined as the concentration which inhibits half of the luciferase activity.

In some embodiments, compounds have an IC₅₀ of 5-20 μM. In some embodiments, compounds have an IC₅₀≤5 μM. In some embodiments, compounds have an IC₅₀≤1 μM. In some embodiments, compounds have an IC₅₀≤0.1 μM. In some embodiments, compounds have an IC₅₀≤0.01 μM. In some embodiments, compounds have an IC₅₀≤0.001 μM.

### Example 1B

### DRE-Luciferase Reporter Assay (Alternate Method)

AHR binds to Dioxin Responsive Elements (DRE) upstream of genes that it activates. One measure of AHR activity is activation of a reporter gene, such as luciferase, downstream of one or multiple DRE elements. Luciferase activity will reflect activation and inhibition of AHR in the cells expressing his reporter.

Murine Hepa1-6 or Hepa-1c1c7 or other murine cell line with a DRE-luciferase reporter either stably or transiently transfected were plated in media in plates (96-well, 384-well or other plates) and incubated overnight at 37° C. in a C02 incubator or compound and agonist were added at the time of plating. Likewise, human HepG2 or other human cell line with a DRE-luciferase reporter either stably or transiently transfected were plated in media in plates (96-well, 384-well or other plates) and incubated overnight at 37° C. in a C02 incubator or compound and agonist were added at the time of plating.

At the time that cells are plated or following incubation overnight, an AHR activating ligand, such as TCDD, kynurenine, ITE (2-(1H-indole-3-ylcarbonyl)-4-thiazolecarboxylic methyl ester), VAF347, BNF (beta-naphthoflavone), FICZ (6-formylindolo(3,2-b) carbazole or other AHR ligands, was added with or without AHR antagonist.

Cells were incubated for 4, 15 or 24 hours or another time point and then lysed for determination of luciferase activity as a read-out of the AHR activation or inhibition. Luciferase was measured with a commercial kit such as the Promega Luciferase kit or any kit or reagents that provide the luciferin substrate for measuring luciferase activity. The level of luciferase with only activating ligand added was the maximum signal while the luciferase with no ligand was the minimum signal. IC₅₀ values were determined as the concentration which inhibits half of the luciferase activity. Compounds assayed and their IC₅₀ values are shown in Table 2, below.

In some embodiments, compounds have an IC₅₀ of 5-20 μM. In some embodiments, compounds have an IC₅₀≤5 μM. In some embodiments, compounds have an IC₅₀≤1 μM. In some embodiments, compounds have an IC₅₀≤0.1 μM. In some embodiments, compounds have an IC₅₀≤0.01 μM. In some embodiments, compounds have an IC₅₀≤0.001 μM.

Activity of certain compounds of the present invention as obtained by the above assay is set forth in Table 2, below.

In Table 2, IC₅₀ values are reported as A, B, C and D, whereby A represents an IC₅₀ of <0.5 μM; B represents an IC₅₀ of between 0.5 and 1.0 μM; and C represents an IC₅₀ of between 1.0 and 1.5 μM; and D represents an IC₅₀ of >1.5 μM.

TABLE 2
IC₅₀ Values for Select
Compounds Assayed
According to Example 1B.

| Compound | IC₅₀ |
|---|---|
| I-1 | D |
| I-2 | D |
| I-3 | D |
| I-4 | A |
| I-5 | D |
| I-6 | D |
| I-7a | D |
| I-7b | C |
| I-8a | C |
| I-8b | C |
| I-9 | A |
| I-10 | A |
| I-11 | A |
| I-12 | C |
| I-13a | D |
| I-13b | D |
| I-14 | C |
| I-15a | D |
| I-15b | D |
| I-16 | D |
| I-17 | B |
| I-18 | A |
| I-19 | D |
| I-20a | C |
| I-20b | A |
| I-21 | D |
| I-22 | A |
| I-23 | A |
| I-24a | D |
| I-24b | D |
| I-25a | A |
| I-25b | A |
| I-26a | D |
| I-26b | D |
| I-27a | D |
| I-27b | D |
| I-29a | D |
| I-29b | D |
| I-30 | B |
| I-31 | A |
| I-32a | A |
| I-32b | A |
| I-33 | A |
| I-34a | D |
| I-34b | D |
| I-35a | C |
| I-35b | B |
| I-36a | D |
| I-36b | D |
| I-37a | B |
| I-37b | A |
| I-38 | A |
| I-39 | D |
| I-40 | D |
| I-41a | A |
| I-41b | A |
| I-42a | A |
| I-42b | A |
| I-43 | D |
| I-44a | D |
| I-45 | C |
| I-44b | D |
| I-47 | A |
| I-48a | D |
| I-48b | D |
| I-49 | D |
| I-50 | D |
| I-51 | A |
| I-52 | B |
| I-53 | B |
| I-54 | D |
| I-55a | D |
| I-55b | D |
| I-56a | A |
| I-56b | A |
| I-58 | A |
| I-59 | A |
| I-60a | D |
| I-60b | A |
| I-61a | A |
| I-61b | B |
| I-62a | D |
| I-62b | D |
| I-63a | D |
| I-63b | D |
| I-64a | A |
| I-64b | A |
| I-65a | A |
| I-65b | A |
| I-66a | D |
| I-66b | D |

### Example 1C

### Mouse Pharmacokinetics Study

Formulations of AHR inhibitor were administered intravenously or orally via gavage to CD-1 mice. Typically, at 0.167, 0.5, 1, 2, 4, 6, 12, and 24 hours post-dose, blood was collected and processed to plasma by centrifugation and stored at −80° C. until analysis.

Internal standard was added to each sample prior to protein precipitation with acetonitrile. The precipitates were filtered through a Phree phospholipid removal filter plate and the samples were analyzed by LC/MS/MS. A standard curve was prepared in plasma from typically from 1.0 ng/mL to 3000 ng/mL and processed in the same manner as the samples. Sample analysis was typically performed on a suitable LC/MS/MS system fitted with an analytical UPLC column and compounds eluted from the analytical column with a gradient from 30-95% 0.1% formic acid (v/v) in ACN: 0.1% formic acid (v/v) in water. Mass spectrometric detection of test compound and the internal standard was performed by MRM in positive mode. The pharmacokinetics of each compound were analyzed by Phoenix WinNonlin software (Pharsight, St. Louis, Mo.) via non-compartmental analysis.

### Example 1D

### In Vitro Mouse Liver S9 Metabolic Stability Assay

CD-I mouse liver S9 were purchased from Corning or XenoTech LLC or BioreclamationIVT, LLC or WuXi prepared. The cells were stored at −80° C. in a freezer before use. β-Nicotinamide adenine dinucleotide phosphate (NADP), Glucose 6-phosphate (G6P), Glucose 6-phosphate dehydrogenase from yeast (G6PDH), Uridine 5′-diphophoglucuronic acid trisodium salt (UDPGA) and Adenosine 3′-phosphate 5′-phosphosulfate lithium salt hydrate (PAPS) were available commercially from Sigma.

Compounds were diluted in DMSO to make 10 mM stock solution. 5 μL of this stock solution (10 mM, DMSO) was diluted with 45 μL DMSO and 450 μL 50% Methanol/Water to make intermediate stock solution (100 μM, 45% MeOH, 10% DMSO). 50 μL of intermediate stock solution was diluted with 450 μL 100 mM phosphate buffer to make a final stock solution (10 μM, 4.5% MeOH, 1% DMSO). 10 uL of final stock solution was added to 90 uL liver S9 system (final concentration of 1 μM, 0.45% MeOH, 0.1% DMSO).

Test compounds were incubated at 37° C. with liver S9 (pooled from multiple donors) at 1 μM in the presence of a NADPH regenerating system, UDPGA, and PAPS at 1 mg/mL S9 protein. Time samples (0 and 60 minutes) were removed and immediately mixed with cold acetonitrile containing internal standard (IS). Samples were analyzed by LC/MS/MS and disappearance of test compounds were assessed based on peak area ratios of analyte/IS (no standard curve). All samples were injected and analyzed using LC-MS/MS. The analyte/internal standard peak area ratios were converted to percentage remaining (% Remaining) with the following equation: % Remaining at 60 min=(Peak area ratio of analyte to IS at 60 min/Peak area ratio of analyte to IS at t=0)×100%.

### Example 1E

### In Vivo Mouse Liver and Spleen Cyp1a1 Modulation Assay

C57BL/6 mice, female, 6-8 weeks old, weighing approximately 18-20 g were purchased from Shanghai Lingchang Biological Technology Co., Ltd or other certified vendors and used in the studies. Animal husbandry, feeding and health conditions are according to animal welfare guidelines. VAG539 (30 mg/kg, po) was used as AHR agonist, and test compounds were formulated in suitable vehicles, typically 0.5% methylcellulose).

C57BL/6 mice (n=3 per group) were treated with AHR agonist alone or with AHR agonist and test compounds. Animals were sacrificed at 4 or 10 hours after treatment upon which their livers and spleens were collected and subsequently analyzed by qPCR. Normalized fold induction of cyp1a1 was determined by comparing mCYP1A1 and mGAPDH counts (ct) according to: normalized fold=₂^{-ΔΔCt}. The percent inhibition was calculated according to:

$$[ 1 - \frac{\begin{matrix}average normalized fold for \\ AHR agonist and compound treated\end{matrix} }{average normalized fold for AHR agonist treated} ] \times 100 = \% inhibition$$

### Example 1F

### T-Cell Study

Human T cells were isolated by CD3 negative selection after isolation of PBMCs from blood of human donors via ficoll density gradient centrifugation. One million T cells were activated with 25 uL of CD3/CD28 tetramer (Stemcell) in the presence or absence of compounds for 24 hours, after which media was removed and stored at −80 C for later cytokine analysis. Cells were then washed 2× with PBS, before isolating RNA according to the manufacturer's instructions for the RNAeasy mini kit (Qiagen).

RNA was converted to cDNA using VILO-IV RT mastermix (Thermofisher), and q-RT-PCR was performed to determine levels of IL-22 (Hs01574154_m1), Cyp1a1 (Hs01054797_g1), and GAPDH (Hs00266705_g1). Data was analyzed using the ddCT method whereby each sample is first normalized to GAPDH housekeeping gene before being normalized to control treatment.

Cytokine levels were determined utilizing the mesoscale discovery (MSD) platform (K15067L-2) and MSD analysis software according to the manufacturer's instructions.

CD3/CD28 activated T cells are AHR activated as measured by gene expression and cytokine production. Treatment with the AHR inhibitor lead to inhibition of cyp1a1 and IL22 gene expression and cytokine IL-22 production. AHR inhibition also increases production of the pro-inflammatory cytokine IL-2.

### Example 1G

### Efficacy Study of AHR Antagonists and Checkpoint Inhibitor Anti-PD-1 in the Mouse Colorectal Cancer Model CT26 in Balb/c Mice

CT26 is a murine colon carcinoma cell line obtained from ATCC. CT26 cells were cultured in RPMI supplemented with 10% FBS. 5×10⁵ CT26 cells in 100 μl PBS were implanted subcutaneously in 6-8 week old female, Balb/c mice. Dosing for the efficacy study starts 4 days post implant: AHR antagonist was dosed orally, every day (QD) at or 10 mg/kg for 3 weeks. Anti-PD-1 (BioXcell RMP1-14) was twice a week, intraperitoneally (IP) at 10 mg/kg for five total doses. Tumors were monitored by caliper measurement every 2-3 days and body weight measured three times per week.

### Example 1H

### Efficacy Study of AHR Antagonists and Checkpoint Inhibitor Anti-PD-1 in the Mouse Melanoma Model B16-IDO in C57BL/6 Mice

B16-IDO is a murine melanoma carcinoma cell line that has been engineered to overexpress IDO1 (Holmgaard, 2015 Cell Reports). B16-IDO cells were cultured in DMEM supplemented with 10% FBS. 2×10⁵ B16-IDO cells in 50 μl PBS were implanted intradermally in 6-8 week old female, C57BL/6 mice. Dosing for the efficacy study starts 7 days post implant: AHR antagonist I-70 was dosed orally, every day (QD) at or 10 mg/kg for 2 weeks. Anti-PD-1 (BioXcell RMP1-14) was administered every 3^{rd} day, intraperitoneally (IP) at 250 μg/mouse for five total doses. Tumors were monitored by caliper measurement every 2-3 days and body weight measured three times per week.

### Example 2

### Synthesis of Compound I-1

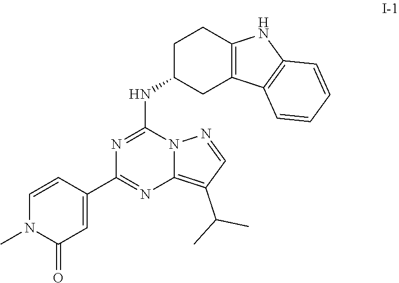 [SMILES: CC(C)c1cnn2c(N[C@@H]3CCc4[nH]c5ccccc5c4C3)nc(-c3ccn(C)c(=O)c3)nc12]

### Synthetic Scheme:

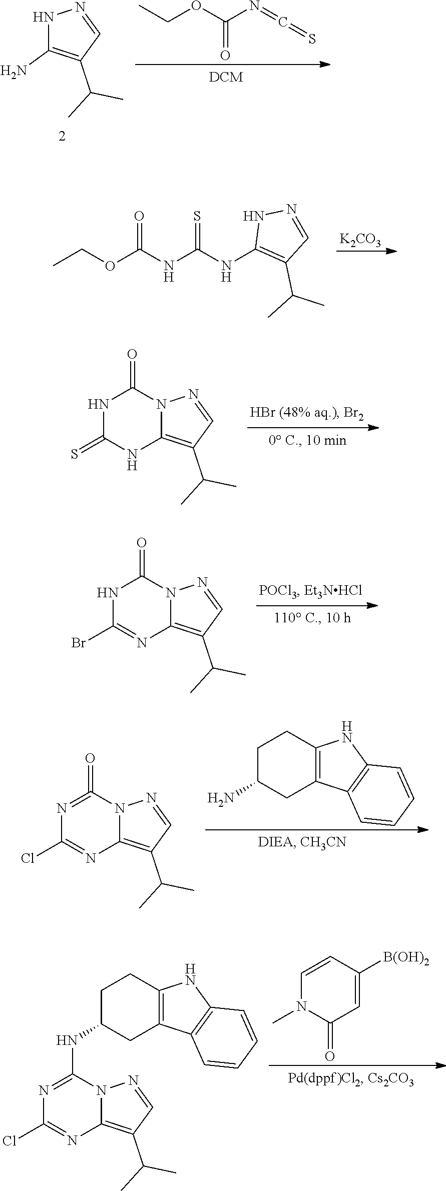

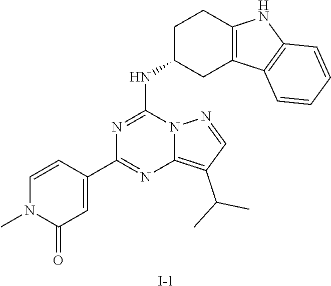 [SMILES: CC(C)c1cnn2c(N[C@@H]3CCc4[nH]c5ccccc5c4C3)nc(-c3ccn(C)c(=O)c3)nc12 ; [I-]]

### Step 1: Ethyl N-[(4-isopropyl-1H-pyrazol-5-yl)carbamothioyl]carbamate

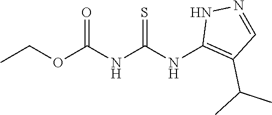 [SMILES: CCOC(=O)NC(=S)Nc1[nH]ncc1C(C)C]

To a solution of 4-isopropyl-3H-pyrazol-3-amine (9 g, 71.90 mmol, 1 eq) in DCM (100 mL) was added ethyl N-(thioxomethylene)carbamate (7.54 g, 57.52 mmol, 6.80 mL, 0.8 eq). The mixture was stirred at 20° C. for 2 h. After concentration, to the residue was added PE/EtOAc (10/1, 150 mL) and stirred at 20° C. for 2 h. The suspension was filtered and the filter cake was washed with PE/EtOAc (10/1, 30 mL×2), dried in vacuo to yield ethyl N-[(4-isopropyl-1H-pyrazol-5-yl)carbamothioyl]carbamate (12 g, 42.13 mmol, 58.6% yield, 90% purity) as a yellow solid. ¹H NMR (400 MHz, CD3OD) δ ppm 7.47 (s, 1H), 4.28 (q, J=7.1 Hz, 2H), 2.92-2.78 (m, 1H), 1.34 (t, J=7.1 Hz, 3H), 1.20 (d, J=6.8 Hz, 6H); ES-LCMS m/z 257.2 [M+H]⁺.

### Step 2: 8-Isopropyl-2-thioxo-1H-pyrazolo[1,5-a][1,3,5]triazin-4-one

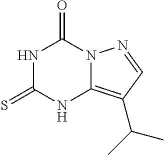

To a suspension of ethyl N-[(4-isopropyl-1H-pyrazol-5-yl)carbamothioyl]carbamate (12 g, 42.13 mmol, 1 eq) in MeCN (300 mL) was added K₂CO₃ (17.47 g, 126.40 mmol, 3 eq). The mixture was stirred at 85° C. for 12 h. The mixture diluted with water (200 mL) and adjusted pH to 7-8 with AcOH. The solvents were evaporated and the residue was suspended in water (500 mL). The solid was filtered off, washed with water (30 mL×2) and dried to yield 8-isopropyl-2-thioxo-1H-pyrazolo[1,5-a][1,3,5]triazin-4-one (8.5 g, 38.41 mmol, 91.2% yield, 95% purity) as a white solid. ¹H NMR (400 MHz, CD3OD) δ ppm 7.83 (s, 1H), 3.04 (td, J=6.9, 13.9 Hz, 1H), 1.25 (d, J=6.8 Hz, 6H); ES-LCMS m/z 211.1 [M+H]+.

### Step 3: 2-Bromo-8-isopropyl-3H-pyrazolo[1,5-a][1,3,5]triazin-4-one

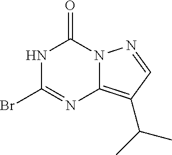

To a solution of 8-isopropyl-2-thioxo-1H-pyrazolo[1,5-a][1,3,5]triazin-4-one (0.9 g, 4.07 mmol, 1 eq) in 48% aqueous HBr (100 mL) cooled to 0° C. was added a solution of Br₂ (1.30 g, 8.13 mmol, 419.26 μL, 2 eq) in 48% aqueous HBr (20 mL) dropwise. The mixture was stirred at 0° C. for 10 min. The mixture was poured into ice cold H₂O (200 mL), neutralized with saturated aqueous Na₂CO₃ solution to pH=7-8. The mixture was extracted with EtOAc (150 mL×3). The combined organic layers were washed with brine (50 mL), dried over Na₂SO₄, filtered and concentrated to yield 2-bromo-8-isopropyl-3H-pyrazolo[1,5-a][1,3,5]triazin-4-one (1 g, 2.76 mmol, 67.9% yield, 71% purity) as a yellow solid. ¹H NMR (400 MHz, CDCl₃) δ ppm 7.93 (br s, 1H), 3.23-3.07 (m, 1H), 1.36-1.23 (m, 6H); ES-LCMS m/z 257.0, 259.0 [M+H]⁺.

### Step 4: 2,4-Dichloro-8-isopropyl-pyrazolo[1,5-a][1,3,5]triazine

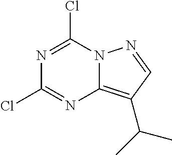

A solution of 2-bromo-8-isopropyl-3H-pyrazolo[1,5-a][1,3,5]triazin-4-one (900 mg, 2.49 mmol, 1 eq), Et₃N.HCl (1.03 g, 7.46 mmol, 3 eq) and POCl₃ (141.5 g, 922.83 mmol, 85.76 mL, 371.28 eq) was stirred at 110° C. for 12 h. The mixture was concentrated. The residue was added DCM (20 mL) and then poured into ice cold water (50 mL). The mixture was extracted with DCM (50 mL×2). The combined organic layers were washed with brine (50 mL), dried over Na₂SO₄, filtered and concentrated. The residue was purified by flash silica gel chromatography (ISCO®; 20 g SepaFlash® Silica Flash Column, Eluent of 0˜20% Ethyl acetate/Petroleum ethergradient 30 mL/min, TLC: (PE/EtOAc=5/1, R_{f}=0.5)) to yield 2,4-dichloro-8-isopropyl-pyrazolo[1,5-a][1,3,5]triazine (650 mg, 2.25 mmol, 90.5% yield, 80% purity) as yellow oil. ¹H NMR (400 MHz, CDCl₃) δ ppm 8.17 (s, 1H), 3.25 (m, 1H), 1.35 (d, J=6.8 Hz, 6H); ES-LCMS m/z 231.1, 233.1 [M+H]⁺.

### Step 5: (3R)—N-(2-Chloro-8-isopropyl-pyrazolo[1,5-a][1,3,5]triazin-4-yl)-2,3,4,9-tetrahydro-1H-carbazol-3-amine

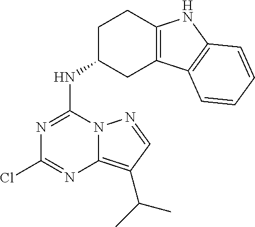 [SMILES: CC(C)c1cnn2c(N[C@@H]3CCc4[nH]c5ccccc5c4C3)nc(Cl)nc12]

To a solution of 2,4-dichloro-8-isopropyl-pyrazolo[1,5-a][1,3,5]triazine (650 mg, 2.25 mmol, 1 eq) in CH₃CN (20 mL) was added DIEA (872.48 mg, 6.75 mmol, 1.18 mL, 3 eq) and (3R)-2,3,4,9-tetrahydro-1H-carbazol-3-amine (419.12 mg, 2.25 mmol, 1 eq). The mixture was stirred at 70° C. for 2 h. The mixture was concentrated. The crude material was purified on silica gel column chromatography (from PE/EtOAc=10/1 to 2/1, TLC: PE/EtOAc=5/1, R_{f}=0.5) to yield (3R)—N-(2-chloro-8-isopropyl-pyrazolo[1,5-a][1,3,5]triazin-4-yl)-2,3,4,9-tetrahydro-1H-carbazol-3-amine (700 mg, 1.81 mmol, 80.5% yield, 98.6% purity) as a yellow solid. ¹H NMR (400 MHz, CDCl₃) δ ppm 7.84 (br s, 1H), 7.81 (s, 1H), 7.45 (d, J=7.6 Hz, 1H), 7.32 (d, J=8.1 Hz, 1H), 7.20-7.14 (m, 1H), 7.13-7.07 (m, 1H), 6.76 (d, J=8.6 Hz, 1H), 4.83 (br s, 1H), 3.29 (dd, J 4.9, 15.4 Hz, 1H), 3.20 (td, J=6.9, 13.8 Hz, 1H), 3.04-2.83 (m, 3H), 2.33-2.19 (m, 2H), 1.30 (d, J=7.1 Hz, 6H); ES-LCMS m/z 381.2 [M+H]⁺.

### Step 6: 4-[8-Isopropyl-4-[[(3R)-2,3,4,9-tetrahydro-1H-carbazol-3-yl]amino]pyrazolo[1,5-a][1,3,5]triazin-2-yl]-1-methyl-pyridin-2-one (I-1)

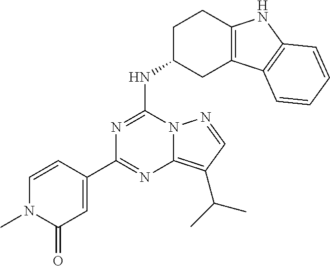 [SMILES: CC(C)c1cnn2c(N[C@@H]3CCc4[nH]c5ccccc5c4C3)nc(-c3ccn(C)c(=O)c3)nc12]

To a mixture of (3R)—N-(2-chloro-8-isopropyl-pyrazolo[1,5-a][1,3,5]triazin-4-yl)-2,3,4,9-tetrahydro-1H-carbazol-3-amine (80 mg, 207.10 μmol, 1 eq), (1-methyl-2-oxo-4-pyridyl)boronic acid (158.38 mg, 1.04 mmol, 5 eq) in 1,4-dioxane (2 mL) and H₂O (0.5 mL) was added Cs₂CO₃ (202.43 mg, 621.31 μmol, 3 eq) and Pd(dppf)Cl₂.DCM (16.91 mg, 20.71 μmol, 0.1 eq) under N₂ atmosphere. The mixture was irradiated and stirred at 110° C. for 0.5 h under microwave. The mixture was concentrated. The crude material was purified by preparative TLC (DCM/MeOH=15/1, R_{f}=0.3) to yield a crude residue, which was purified by preparative HPLC (column: Agela ASB 150*25 mm*5 μm; mobile phase: [water (0.05% HCl)−ACN]; B %: 65%-95%, 8 min), followed by lyophilization to yield 4-[8-isopropyl-4-[[(3R)-2,3,4,9-tetrahydro-1H-carbazol-3-yl]amino]pyrazolo[1,5-a][1,3,5]triazin-2-yl]-1-methyl-pyridin-2-one (17.55 mg, 35.67 μmol, 17.2% yield, 99.6% purity, HCl, optical rotation: [α]^{22.1}_{D}=+11.553 (MeOH, c=0.034 g/100 mL)) as a yellow solid. ¹H NMR (400 MHz, CD3OD+Na₂CO₃) δ ppm 7.97 (s, 1H), 7.69 (d, J=7.1 Hz, 1H), 7.61 (d, J=1.5 Hz, 1H), 7.36 (dt, J=2.7, 4.8 Hz, 2H), 7.27 (d, J=8.1 Hz, 1H), 7.03 (t, J=7.1 Hz, 1H), 6.97-6.92 (m, 1H), 4.81-4.77 (m, 1H), 3.59 (s, 3H), 3.29-3.20 (m, 2H), 3.14-2.83 (m, 3H), 2.36 (br s, 1H), 2.31-2.15 (m, 1H), 1.40 (d, J=7.1 Hz, 6H); ES-LCMS m/z 454.2 [M+H]⁺.

### Example 3

### Synthesis of Compound I-2

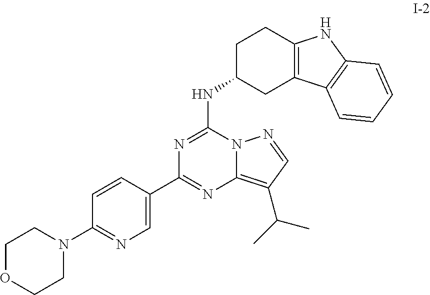 [SMILES: CC(C)c1cnn2c(N[C@@H]3CCc4[nH]c5ccccc5c4C3)nc(-c3ccc(N4CCOCC4)nc3)nc12]

### Synthetic Scheme:

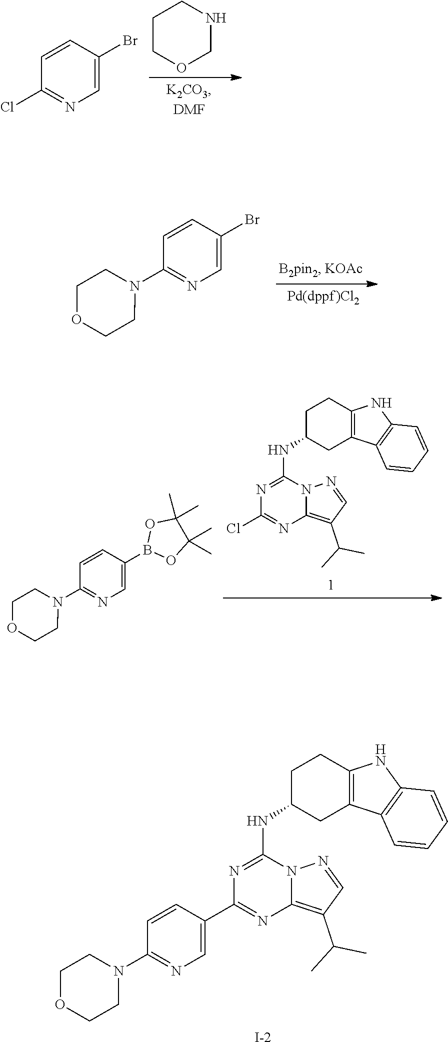

### Step 1: 4-(5-Bromo-2-pyridyl)morpholine

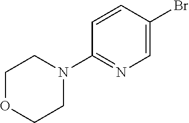 [SMILES: Brc1ccc(N2CCOCC2)nc1]

A mixture of 5-bromo-2-chloro-pyridine (850 mg, 4.42 mmol, 1 eq), morpholine (577.21 mg, 6.63 mmol, 583.04 μL, 1.5 eq) and Cs₂CO₃ (4.32 g, 13.25 mmol, 3 eq) in DMF (10 mL) was stirred at 120° C. for 12 h under N₂ atmosphere. The mixture was filtered and concentrated. The residue was purified on silica gel column chromatography (from PE/EtOAc=I/O to 3/1, TLC: PE/EtOAc=3/1, R_{f}=0.5) to yield 4-(5-bromo-2-pyridyl)morpholine (420 mg, 1.64 mmol, 37.2% yield, 95% purity) as a white solid. ¹H NMR (400 MHz, CDCl₃) δ ppm 8.19 (d, J=2.4 Hz, 1H), 7.54 (dd, J=2.6, 8.9 Hz, 1H), 6.52 (d, J=9.0 Hz, 1H), 3.79 (d, J=5.1 Hz, 4H), 3.44 (d, J=4.9 Hz, 4H); ES-LCMS m/z 243.0, 245.1 [M+H]⁺.

### Step 2: 4-[5-(4,4,5,5-Tetramethyl-1,3,2-dioxaborolan-2-yl)-2-pyridyl]morpholine

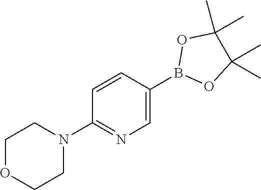 [SMILES: CC1(C)OB(c2ccc(N3CCOCC3)nc2)OC1(C)C]

A mixture of 4-(5-bromo-2-pyridyl)morpholine (370 mg, 1.45 mmol, 1 eq), 4,4,5,5-tetramethyl-2-(4,4,5,5-tetramethyl-1,3,2-dioxaborolan-2-yl)-1,3,2-dioxaborolane (403.89 μmg, 1.59 mmol, 1.1 eq), KOAc (425.70 mg, 4.34 mmol, 3 eq) and Pd(dppf)Cl₂.DCM (118.08 mg, 144.59 μmol, 0.1 eq) in 1,4-dioxane (6 mL) was stirred at 100° C. for 2 h under N₂ atmosphere. The mixture was filtered and concentrated. The residue was purified by flash silica gel chromatography (ISCO®; 12 g SepaFlash® Silica Flash Column, Eluent of 0˜30% Ethyl acetate/Petroleum ethergradient @ 40 mL/min, TLC: PE/EtOAc=3/1, R_{f}=0.4) to yield 4-[5-(4,4,5,5-tetramethyl-1,3,2-dioxaborolan-2-yl)-2-pyridyl]morpholine (380 mg, 1.18 mmol, 81.5% yield, 90% purity) as a yellow solid. ¹H NMR (400 MHz, CDCl₃) δ ppm 8.53 (d, J=1.0 Hz, 1H), 7.82 (dd, J=1.7, 8.6 Hz, 1H), 6.56 (d, J=8.3 Hz, 1H), 3.80-3.77 (m, 4H), 3.56 (d, J=5.1 Hz, 4H), 1.28-1.23 (m, 12H); ES-LCMS m/z 291.2 [M+H]⁺.

### Step 3: (3R)—N-[8-Isopropyl-2-(6-morpholino-3-pyridyl)pyrazolo[1,5-a][1,3,5]triazin-4-yl]-2,3,4,9-tetrahydro-1H-carbazol-3-amine (I-2)

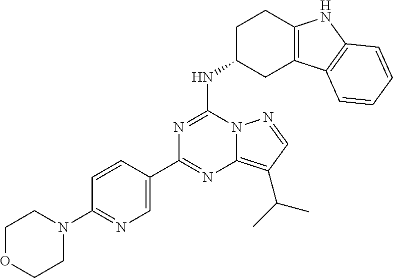 [SMILES: CC(C)c1cnn2c(N[C@@H]3CCc4[nH]c5ccccc5c4C3)nc(-c3ccc(N4CCOCC4)nc3)nc12]

A mixture of (3R)—N-(2-chloro-8-isopropyl-pyrazolo[1,5-a][1,3,5]triazin-4-yl)-2,3,4,9-tetrahydro-1H-carbazol-3-amine (100 mg, 258.88 μmol, 1 eq), 4-[5-(4,4,5,5-tetramethyl-1,3,2-dioxaborolan-2-yl)-2-pyridyl]morpholine (250.39 mg, 776.64 μmol, 3 eq), Cs₂CO₃ (253.04 mg, 776.64 μmol, 3 eq) and Pd(dppf)Cl₂ (18.94 mg, 25.89 μmol, 0.1 eq) were taken up into a microwave tube in 1,4-dioxane (4 mL) and H₂O (1 mL) under N₂ atmosphere. The sealed tube was heated at 110° C. for 1 h under microwave. The reaction mixture was filtered and concentrated to yield a residue which was purify with preparative TLC (PE/EtOAc=1/1, R_{f}=0.6) to yield as a yellow solid. The solid was purified by preparative HPLC (column: Agela ASB 150*25 mm*5 μm; mobile phase: [water (0.05% HCl)−ACN]; B %: 50%-80%, 8 min), followed by lyophilization to yield (3R)—N-[8-isopropyl-2-(6-morpholino-3-pyridyl)pyrazolo[1,5-a][1,3,5]triazin-4-yl]-2,3,4,9-tetrahydro-1H-carbazol-3-amine (50.7 mg, 87.18 μmol, 33.7% yield, 100% purity, 2 HCl) as a yellow solid. ¹H NMR (400 MHz, CD3OD) δ ppm 8.95 (dd, J=2.1, 9.7 Hz, 1H), 8.83 (d, J=2.0 Hz, 1H), 8.01 (s, 1H), 7.48 (d, J=9.8 Hz, 1H), 7.39 (d, J=7.8 Hz, 1H), 7.30 (d, J=8.0 Hz, 1H), 7.06 (t, J=7.0 Hz, 1H), 7.01-6.95 (m, 1H), 4.86-4.78 (m, 1H), 3.92-3.86 (m, 4H), 3.79-3.73 (m, 4H), 3.32-3.24 (m, 2H), 3.08-2.88 (m, 3H), 2.44-2.36 (m, 1H), 2.35-2.23 (m, 1H), 1.42 (d, J=7.0 Hz, 6H); ES-LCMS m/z 509.3 [M+H]⁺.

### Example 4

### Synthesis of Compound I-3

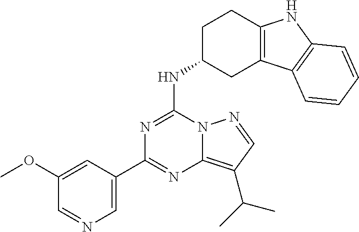 [SMILES: COc1cncc(-c2nc(N[C@@H]3CCc4[nH]c5ccccc5c4C3)n3ncc(C(C)C)c3n2)c1]

### Synthetic Scheme:

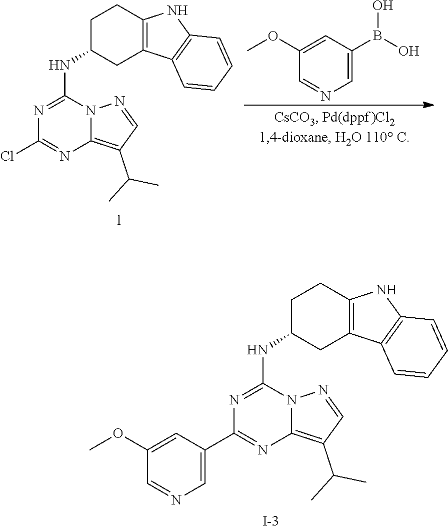

### Step 1: (3R)—N-[8-Isopropyl-2-(5-methoxy-3-pyridyl)pyrazolo[1,5-a][1,3,5]triazin-4-yl]-2,3,4,9-tetrahydro-1H-carbazol-3-amine (I-3)

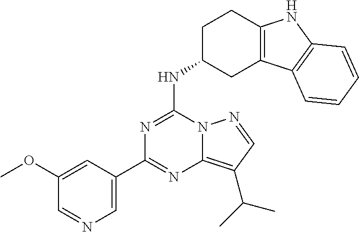 [SMILES: COc1cncc(-c2nc(N[C@@H]3CCc4[nH]c5ccccc5c4C3)n3ncc(C(C)C)c3n2)c1]

A mixture of (3R)—N-(2-chloro-8-isopropyl-pyrazolo[1,5-a][1,3,5]triazin-4-yl)-2,3,4,9-tetrahydro-1H-carbazol-3-amine (70 mg, 181.22 μmol, 1 eq), (5-methoxy-3-pyridyl)boronic acid (83.15 mg, 543.65 μmol, 3 eq), Cs₂CO₃ (177.13 mg, 543.65 umol, 3 eq) and Pd(dppf)Cl₂ (13.26 mg, 18.12 μmol, 0.1 eq) were taken up into a microwave tube in 1,4-dioxane (4 mL) and H₂O (1 mL) under N₂ atmosphere. The sealed tube was heated at 110° C. for 30 min under microwave. The mixture was filtered and concentrated. The residue was purified with preparative TLC (PE/EtOAc=3/1, R_{f}=0.6) to yield a residue which was purified by preparative HPLC (column: Agela ASB 150*25 mm*5 μm; mobile phase: [water (0.05% HCl)−ACN]; B %: 55%-85%, 8 min), followed by lyophilization to yield (3R)—N-[8-isopropyl-2-(5-methoxy-3-pyridyl)pyrazolo[1,5-a][1,3,5]triazin-4-yl]-2,3,4,9-tetrahydro-1H-carbazol-3-amine (25.42 mg, 48.28 μmol, 26.6% yield, 100% purity, 2 HCl, Optical rotation ([α]^{23.2}_{D}=+35.783, (MeOH, c=0.059 g/100 mL))) as a yellow solid. ¹H NMR (400 MHz, CD3OD) δ ppm 9.28 (s, 1H), 8.95 (d, J 1.3 Hz, 1H), 8.62 (d, J=2.8 Hz, 1H), 8.06 (s, 1H), 7.38 (d, J=7.8 Hz, 1H), 7.29 (d, J=8.0 Hz, 1H), 7.06 (t, J=7.5 Hz, 1H), 7.00-6.95 (m, 1H), 4.88 (m, 1H), 4.10 (s, 3H), 3.32 (m, 2H), 3.08-2.89 (m, 3H), 2.39 (m, 1H), 2.35-2.23 (m, 1H), 1.44 (d, J=7.0 Hz, 6H); ES-LCMS m/z 454.3 [M+H]⁺.

### Example 5

### Synthesis of Compound I-4

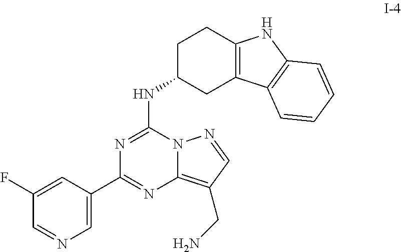 [SMILES: NCc1cnn2c(N[C@@H]3CCc4[nH]c5ccccc5c4C3)nc(-c3cncc(F)c3)nc12]

### Synthetic Scheme:

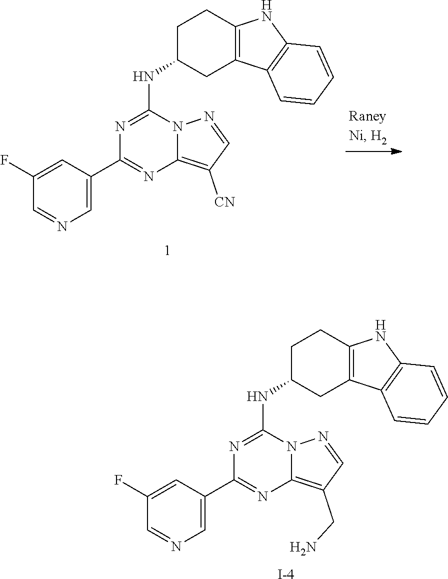

### Step 1: (3R)—N-[8-(Aminomethyl)-2-(5-fluoro-3-pyridyl)pyrazolo[1,5-a][1,3,5]triazin-4-yl]-2,3,4,9-tetrahydro-1H-carbazol-3-amine (I-4)

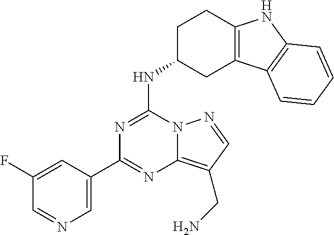 [SMILES: NCc1cnn2c(N[C@@H]3CCc4[nH]c5ccccc5c4C3)nc(-c3cncc(F)c3)nc12]

To a solution of 2-(5-fluoro-3-pyridyl)-4-[[(3R)-2,3,4,9-tetrahydro-1H-carbazol-3-yl]amino]pyrazolo[1,5-a][1,3,5]triazine-8-carbonitrile (100 mg, 218.17 mmol, 1 eq) in MeOH (10 mL) was added Raney-Ni and NH₃.H₂O (27.31 mg, 218.17 mmol, 30.01 mL, 28% purity, 1 eq). The mixture was stirred at 60° C. for 2 h under H2 atmosphere (15 psi). The mixture was filtered, washed with MeOH (10 mL). The filtrate was concentrated to yield a residue which was purified by preparative HPLC (column: Xtimate C18 150*25 mm*5 mm; mobile phase: [water (0.05% HCl)−ACN]; B %: 25%-55%, 7 min), followed by lyophilization to yield (3R)—N-[8-(aminomethyl)-2-(5-fluoro-3-pyridyl)pyrazolo[1,5-a][1,3,5]triazin-4-yl]-2,3,4,9-tetrahydro-1H-carbazol-3-amine (25 mg, 46.48 mmol, 21.3% yield, 100% purity, 3HCl, Optical rotation ([α]^{23.9}_{D}=+30.552, (MeOH, c=0.040 g/100 mL))) as a yellow solid. ¹H NMR (400 MHz, CD3OD) δ ppm 9.52 (s, 1H), 8.82-8.74 (m, 1H), 8.72 (d, J=2.2 Hz, 1H), 8.23 (s, 1H), 7.39 (d, J=7.8 Hz, 1H), 7.29 (d, J=8.1 Hz, 1H), 7.08-7.02 (m, 1H), 7.00-6.94 (m, 1H), 4.87-4.82 (m, 1H), 4.38 (s, 2H), 3.30-3.26 (m, 1H), 3.18-2.91 (m, 3H), 2.40 (m, 1H), 2.35-2.22 (m, 1H); ES-LCM S m/z 412.2 [M-NH₃+H]⁺.

### Example 6

### Synthesis of Compound I-5

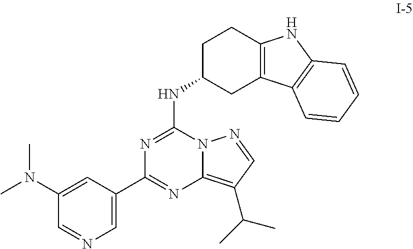 [SMILES: CC(C)c1cnn2c(N[C@@H]3CCc4[nH]c5ccccc5c4C3)nc(-c3cncc(N(C)C)c3)nc12]

### Synthetic Scheme:

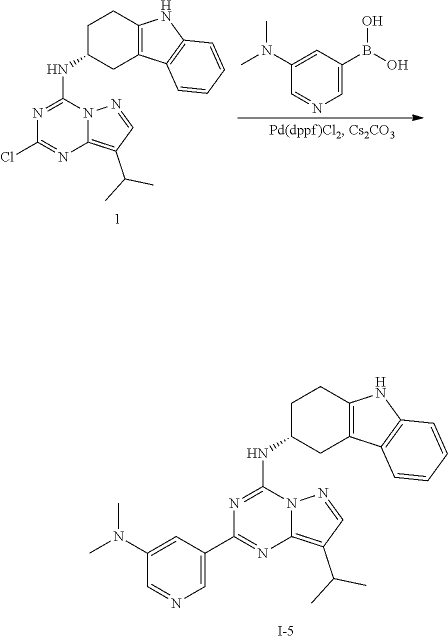

### Step 1: (3R)—N-[2-[5-(Dimethylamino)-3-pyridyl]-8-isopropyl-pyrazolo[1,5-a][1,3,5]triazin-4-yl]-2,3,4,9-tetrahydro-1H-carbazol-3-amine (I-5)

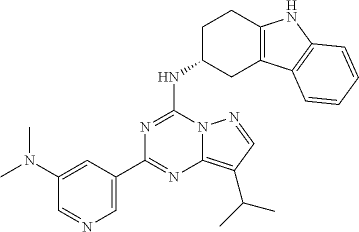 [SMILES: CC(C)c1cnn2c(N[C@@H]3CCc4[nH]c5ccccc5c4C3)nc(-c3cncc(N(C)C)c3)nc12]

To a solution of (3R)—N-(2-chloro-8-isopropyl-pyrazolo[1,5-a][1,3,5]triazin-4-yl)-2,3,4,9-tetrahydro-1H-carbazol-3-amine (60 mg, 155.33 mmol, 1 eq) and [5-(dimethylamino)-3-pyridyl]boronic acid (51.56 mg, 310.65 mmol, 2.0 eq) in 1,4-dioxane (2 mL) and H₂O (0.5 mL) was added Pd(dppf)Cl₂ (11.37 mg, 15.53 mmol, 0.1 eq) and Cs₂CO₃ (151.83 mg, 465.98 mmol, 3.0 eq). The mixture was purged with N₂ for 1 min and stirred at 110° C. for 0.5 h under microwave. The reaction mixture was filtered and concentrated under reduced pressure to yield a residue which was purified by preparative TLC (TLC: PE/EtOAc=1/2, R_{f}=0.29) and then purified by preparative HPLC (column: Xtimate C18 150*25 mm*5 um; mobile phase: [water (0.05% HCl)−ACN]; B %: 40%-70%, 8 min), followed by lyophilization to yield (3R)—N-[2-[5-(dimethylamino)-3-pyridyl]-8-isopropyl-pyrazolo[1,5-a][1,3,5]triazin-4-yl]-2,3,4,9-tetrahydro-1H-carbazol-3-amine (49.05 mg, 85.08 vmol, 54.8% yield, 99.9% purity, 3HCl, Optical rotation ([α]^{23.0}_{D}=+58.214, (MeOH, c=0.099 g/100 mL))) as a yellow solid. ¹H NMR (400 MHz, CD3OD) δ ppm 8.77 (s, 1H), 8.58 (br s, 1H), 8.10 (d, J=2.8 Hz, 1H), 7.99 (s, 1H), 7.33 (d, J=7.8 Hz, 1H), 7.24 (d, J=8.0 Hz, 1H), 7.04-6.97 (m, 1H), 6.96-6.88 (m, 1H), 4.77 (m, 1H), 3.31 (m, 1H), 3.27-3.20 (m, 1H), 3.12 (s, 6H), 3.03-2.83 (m, 3H), 2.41-2.31 (m, 1H), 2.30-2.17 (m, 1H), 1.38 (d, J=7.0 Hz, 6H); ES-LCMS m/z 467.3 [M+H]⁺.

### Example 7

### Synthesis of Compound I-6

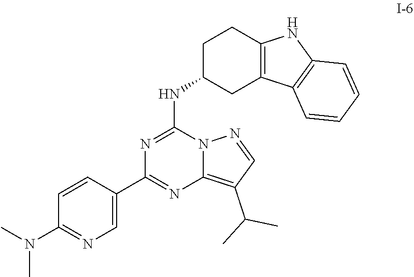 [SMILES: CC(C)c1cnn2c(N[C@@H]3CCc4[nH]c5ccccc5c4C3)nc(-c3ccc(N(C)C)nc3)nc12]

### Synthetic Scheme:

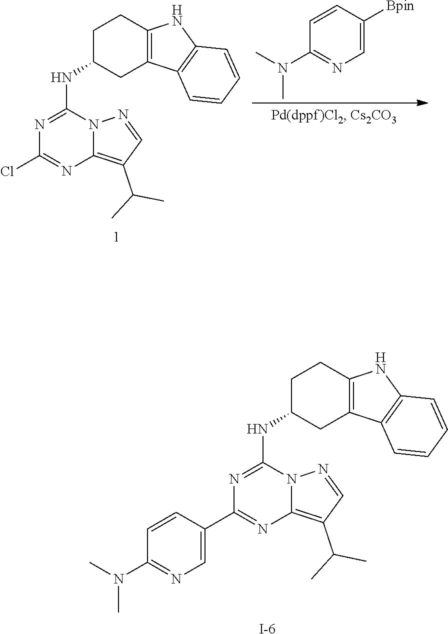

### Step 1: (3R)—N-[2-[6-(dimethylamino)-3-pyridyl]-8-isopropyl-pyrazolo[1,5-a][1,3,5]triazin-4-yl]-2,3,4,9-tetrahydro-H-carbazol-3-amine (I-6)

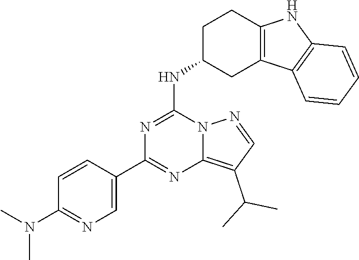 [SMILES: CC(C)c1cnn2c(N[C@@H]3CCc4[nH]c5ccccc5c4C3)nc(-c3ccc(N(C)C)nc3)nc12]

To a mixture of (3R)—N-(2-chloro-8-isopropyl-pyrazolo[1,5-a][1,3,5]triazin-4-yl)-2,3,4,9-tetrahydro-1H-carbazol-3-amine (80 mg, 207.10 mmol, 1 eq) in 1,4-dioxane (1.2 mL) and H₂O (0.3 mL) was added N,N-dimethyl-5-(4,4,5,5-tetramethyl-1,3,2-dioxaborolan-2-yl) pyridin-2-amine (51.39 mg, 207.10 mmol, 1 eq), Cs₂CO₃ (202.43 mg, 621.30 mmol, 3 eq), Pd(dppf)Cl₂ (15.15 mg, 20.71 mmol, 0.1 eq). The mixture was stirred at 110° C. for 0.5 h under microwave. The reaction mixture was filtered and concentrated under reduced pressure to yield a residue which was purified by preparative HPLC (HCl condition; column: Xtimate C18 150*25 mm*5 um; mobile phase: [water (0.05% HCl)−ACN]; B %: 45%-75%, 8 min) to yield (3R)—N-[2-[6-(dimethylamino)-3-pyridyl]-8-isopropyl-pyrazolo[1,5-a][1,3,5]triazin-4-yl]-2,3,4,9-tetrahydro-1H-carbazol-3-amine (50.43 mg, 92.63 mmol, 44.7% yield, 99.1% purity, 2HCl, [α]^{23.2}_{D}=+117.135 (MeOH, c=0.100 g/100 mL)) as a yellow solid. ¹H NMR (400 MHz, CD3OD) δ ppm 8.82 (dd, J=2.0, 9.5 Hz, 1H), 8.74 (d, J=1.7 Hz, 1H), 7.95 (s, 1H), 7.34 (d, J=7.8 Hz, 1H), 7.28-7.21 (m, 2H), 7.07-6.98 (m, 1H), 6.97-6.89 (m, 1H), 4.76 (s, 1H), 3.30 (s, 6H), 3.28-3.19 (m, 2H), 3.06-2.82 (m, 3H), 2.42-2.13 (m, 2H), 1.38 (d, J=6.8 Hz, 6H); ES-LCMS m/z 467.3 [M+H]⁺.

### Example 8

### Synthesis of Compound I-7a, I-7b and I-7c

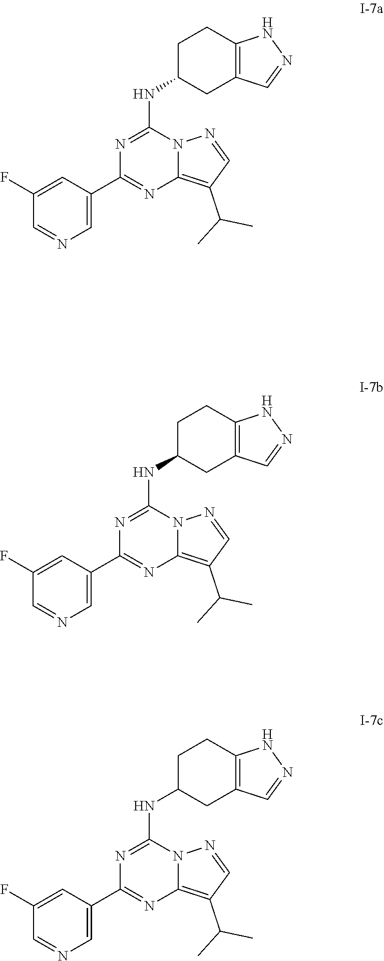 [SMILES: CC(C)c1cnn2c(N[C@@H]3CCc4[nH]ncc4C3)nc(-c3cncc(F)c3)nc12 ; CC(C)c1cnn2c(N[C@H]3CCc4[nH]ncc4C3)nc(-c3cncc(F)c3)nc12 ; CC(C)c1cnn2c(NC3CCc4[nH]ncc4C3)nc(-c3cncc(F)c3)nc12]

### Synthetic Scheme:

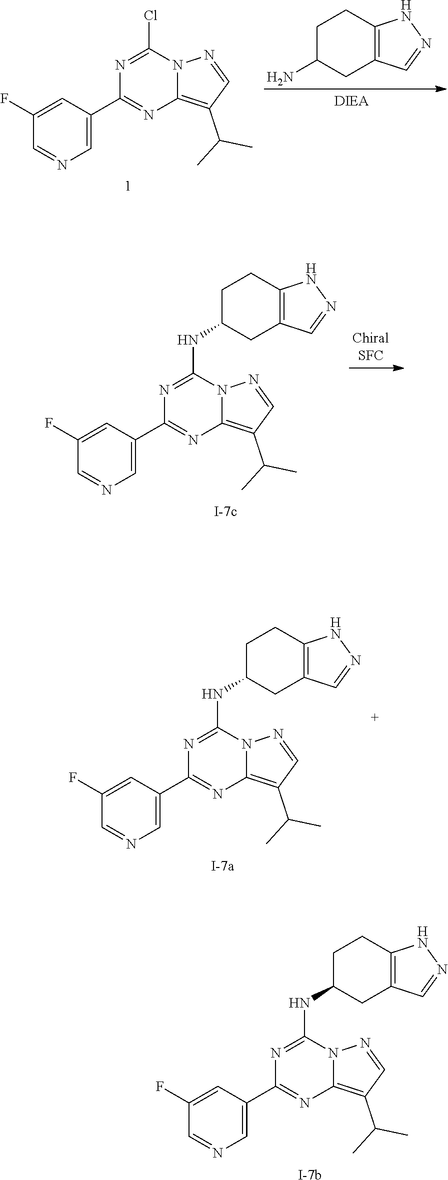

### Step 1: 2-(5-fluoro-3-pyridyl)-8-isopropyl-N-[(5R)-4,5,6,7-tetrahydro-1H-indazol-5-yl]pyrazolo[1,5-a][1,3,5]triazin-4-amine (I-7a) & 2-(5-fluoro-3-pyridyl)-8-isopropyl-N-[(5S)-4,5,6,7-tetrahydro-1H-indazol-5-yl]pyrazolo[1,5-a][1,3,5]triazin-4-amine (I-7b)

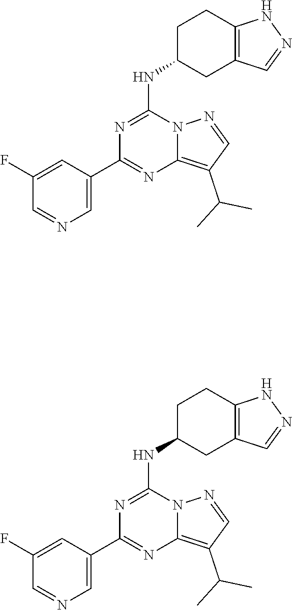 [SMILES: CC(C)c1cnn2c(N[C@@H]3CCc4[nH]ncc4C3)nc(-c3cncc(F)c3)nc12 ; CC(C)c1cnn2c(N[C@H]3CCc4[nH]ncc4C3)nc(-c3cncc(F)c3)nc12]

To a solution of 4-chloro-2-(5-fluoro-3-pyridyl)-8-isopropyl-pyrazolo[1,5-a][1,3,5]triazine (150 mg, 500.84 μmol, 1 eq) in i-PrOH (5 mL) was added DIEA (323.64 mg, 2.50 mmol, 436.17 μL, 5 eq) and 4,5,6,7-tetrahydro-1H-indazol-5-amine (110.49 mg, 525.88 μmol, 1.05 eq, 2HCl). The mixture was stirred at 60° C. for 2 h. TLC (PE/EtOAc=1/1, R_{f}=0.20) showed starting material was consumed completely and one new spot was detected. The reaction mixture was concentrated under reduced pressure to yield a residue which was purified by flash silica gel chromatography (from PE/EtOAc=100/1 to 4/3, TLC: PE/EtOAc=1/1, R_{f}=0.20) to yield the target which separated by SFC (column: DAICEL CHIRALPAK IC (250 mm*30 mm, 5 μm); mobile phase: [0.1% NH₃H₂O EtOH]; B %: 50%-50%), followed by lyophilization to yield an enantiomer (86.21 mg, 218.14 μmol, 43.6% yield, 99.3% purity, SFC: Rₜ=3.953 min, ee=100.0% and [α]^{22.7}_{D}=+2.795, CHCl₃, c=0.103 g/100 mL) as a white solid. ¹H NMR (400 MHz, CDCl₃) δ ppm 9.50 (s, 1H), 8.56 (d, J=2.8 Hz, 1H), 8.48-8.39 (m, 1H), 7.87 (s, 1H), 7.41 (s, 1H), 6.64 (d, J=8.0 Hz, 1H), 4.88-4.75 (m, 1H), 3.35-3.16 (m, 2H), 2.98-2.92 (m, 2H), 2.78 (dd, J=7.5, 15.3 Hz, 1H), 2.36-2.28 (m, 1H), 2.27-2.16 (m, 1H), 1.41 (d, J=6.8 Hz, 6H); ES-LCMS m/z 393.2 [M+H]⁺ and the other enantiomer (85.9 mg, 218.89 μmol, 43.7% yield, 100.0% purity, SFC: Rₜ=4.485 min, ee=97.18% and [α]^{22.8}_{D}=−5.324, CHCl₃, c=0.113 g/100 mL) as a white solid. ¹H NMR (400 MHz, CDCl₃) δ ppm 9.50 (s, 1H), 8.56 (d, J=2.8 Hz, 1H), 8.44 (td, J=2.2, 9.6 Hz, 1H), 7.87 (s, 1H), 7.41 (s, 1H), 6.64 (d, J=8.0 Hz, 1H), 4.81 (t, J=8.0 Hz, 1H), 3.36-3.16 (m, 2H), 2.95 (t, J=6.4 Hz, 2H), 2.78 (dd, J=7.7, 15.2 Hz, 1H), 2.38-2.16 (m, 2H), 1.41 (d, J=6.8 Hz, 6H); ES-LCMS m/z 393.2 [M+H]⁺.

### Example 9

### Synthesis of Compound I-8a, I-8b and I-8c

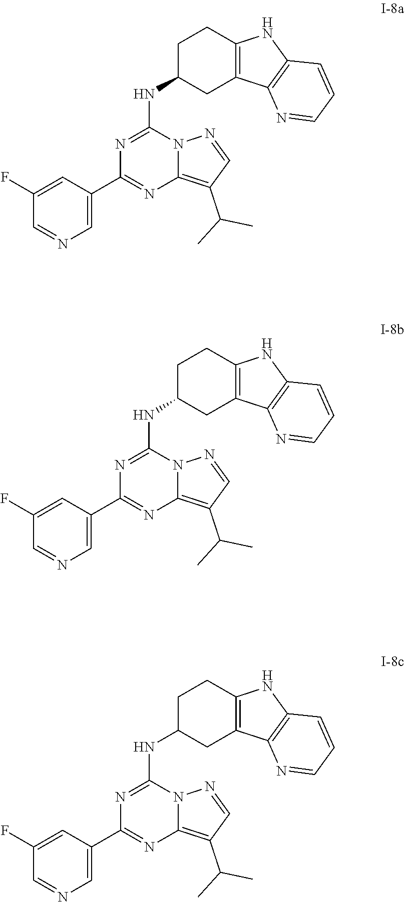 [SMILES: CC(C)c1cnn2c(N[C@H]3CCc4[nH]c5cccnc5c4C3)nc(-c3cncc(F)c3)nc12 ; CC(C)c1cnn2c(N[C@@H]3CCc4[nH]c5cccnc5c4C3)nc(-c3cncc(F)c3)nc12 ; CC(C)c1cnn2c(NC3CCc4[nH]c5cccnc5c4C3)nc(-c3cncc(F)c3)nc12]

### Synthetic Scheme:

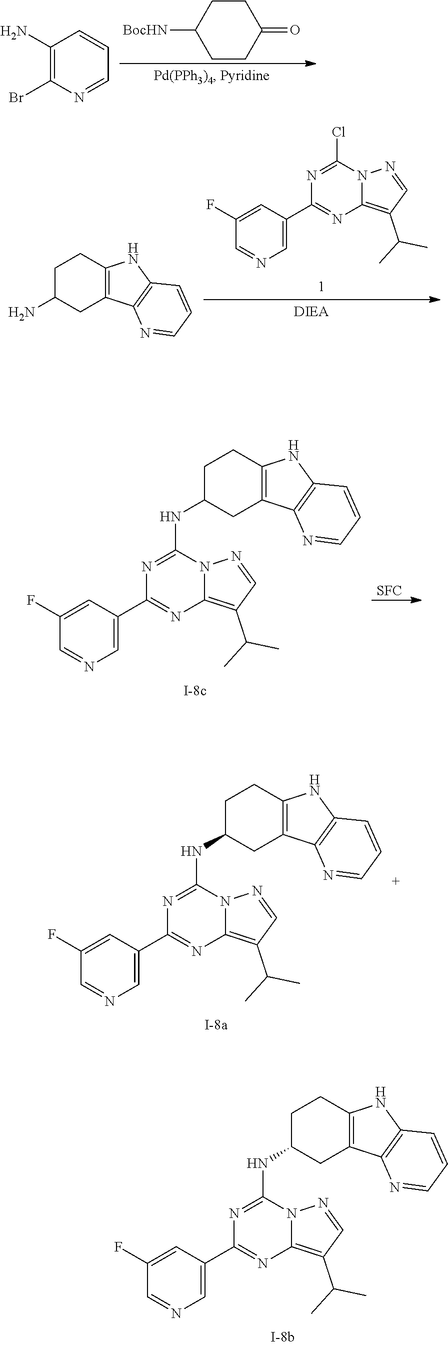

### Step 1: 6,7,8,9-Tetrahydro-5H-pyrido[3,2-b]indol-8-amine

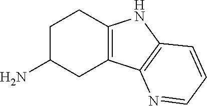 [SMILES: NC1CCc2[nH]c3cccnc3c2C1]

A solution of 2-bromopyridin-3-amine (1 g, 5.78 mmol, 1 eq) in pyridine (3.5 mL) was added Pd(PPh₃)₄ (333.96 mg, 289.00 μmol, 0.05 eq) and tert-butyl N-(4-oxocyclohexyl)carbamate (1.48 g, 6.94 mmol, 1.48 mL, 1.2 eq) was taken up into a microwave tube and then purged with N₂ for 1 min. The sealed tube was heated at 160° C. for 2.5 h under microwave (12 bar). The mixture was concentrated and then water (80 mL) was added. The mixture was extracted with EtOAc (50 mL×3). The combined water layers were concentrated, dissolved in EtOH (50 mL). The reaction mixture was filtered, and the filtrate was concentrated under pressure to yield 6,7,8,9-tetrahydro-5H-pyrido[3,2-b]indol-8-amine (1 g, 4.27 mmol, 73.9% yield, 80.0% purity) as a black solid which was used in the next step without further purification. ¹H NMR (400 MHz, CD3OD) δ ppm 8.12 (dd, J=1.2, 4.9 Hz, 1H), 7.62 (dd, J=1.0, 8.1 Hz, 1H), 7.00 (dd, J=4.9, 8.1 Hz, 1H), 4.81-4.78 (m, 1H), 3.24-3.06 (m, 2H), 2.91-2.80 (m, 2H), 2.12-2.03 (m, 1H), 1.83-1.69 (m, 1H); ES-LCMS m/z 188.1 [M+H]⁺.

### Step 2: (8S)—N-[2-(5-Fluoro-3-pyridyl)-8-isopropyl-pyrazolo[1,5-a][1,3,5]triazin-4-yl]-6,7,8,9-tetrahydro-5H-pyrido[3,2-b]indol-8-amine (I-8a) and (8R)—N-[2-(5-Fluoro-3-pyridyl)-8-isopropyl-pyrazolo[1,5-a][1,3,5]triazin-4-yl]-6,7,8,9-tetrahydro-5H-pyrido[3,2-b]indol-8-amine (I-8b)

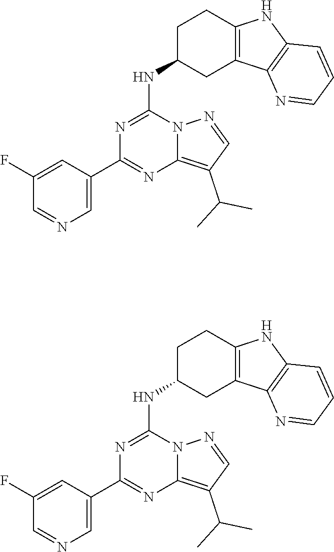 [SMILES: CC(C)c1cnn2c(N[C@H]3CCc4[nH]c5cccnc5c4C3)nc(-c3cncc(F)c3)nc12 ; CC(C)c1cnn2c(N[C@@H]3CCc4[nH]c5cccnc5c4C3)nc(-c3cncc(F)c3)nc12]

To a solution of 4-chloro-2-(5-fluoro-3-pyridyl)-8-isopropyl-pyrazolo[1,5-a][1,3,5]triazine (150 mg, 501.35 μmol, 1 eq) in i-PrOH (15 mL) was added DIEA (194.38 mg, 1.50 mmol, 261.97 μL, 3 eq) and 6,7,8,9-tetrahydro-5H-pyrido[3,2-b]indol-8-amine (140.81 mg, 601.62 μmol, 1.2 eq). The mixture was stirred at 60° C. for 2 h under N₂ atmosphere. The reaction mixture was concentrated under reduced pressure to yield a residue which was purified on silica gel column chromatography (from PE/EtOAc=1/0 to 1/1, TLC: PE/EtOAc=1/1, R_{f}=0.2) to yield a product which was purified by chiral SFC (column: YMC CHIRAL Amylose-C (250 mm*30 mm, 5 μm; mobile phase: [0.1% NH₃H₂O MeOH]; B %: 55%-55%), followed by lyophilization to yield an enantiomer (30 mg, 66.65 μmol, 13.2% yield, 98.3% purity, SFC: Rₜ=1.536 min, ee=97.2% and [α]^{22.7}_{D}=−37.469, MeOH, c=0.050 g/100 mL)) was obtained as a white solid. ¹H NMR (400 MHz, CD3OD) δ ppm 9.40 (s, 1H), 8.56-8.47 (m, 2H), 8.24 (d, J=4.4 Hz, 1H), 7.98 (s, 1H), 7.85 (d, J=7.8 Hz, 1H), 7.19 (dd, J=5.1, 8.1 Hz, 1H), 4.86-4.79 (m, 1H), 3.48 (dd, J=5.3, 15.3 Hz, 1H), 3.30-3.24 (m, 1H), 3.18-2.95 (m, 3H), 2.42 (m, 1H), 2.38-2.23 (m, 1H), 1.42 (d, J=6.8 Hz, 6H); ES-LCMS m/z 443.2 [M+H]⁺; and the other enantiomer (30 mg, 67.80 μmol, 13.5% yield, 100% purity, SFC: Rₜ=1.724 min, ee=98.2% and [α]^{22.7}_{D}=+41.698, MeOH, c=0.051 g/100 mL) was obtained as a white solid. ¹H NMR (400 MHz, CD3OD) δ ppm 9.40 (s, 1H), 8.56-8.46 (m, 2H), 8.24 (d, J=4.9 Hz, 1H), 7.98 (s, 1H), 7.85 (d, J=8.1 Hz, 1H), 7.19 (dd, J=5.0, 8.2 Hz, 1H), 4.85-4.81 (m, 1H), 3.48 (dd, J=5.4, 15.2 Hz, 1H), 3.29-3.24 (m, 1H), 3.18-2.95 (m, 3H), 2.44 (m, 1H), 2.38-2.23 (m, 1H), 1.42 (d, J=6.8 Hz, 6H); ES-LCM S m/z 443.2 [M+H]⁺.

### Example 10

### Synthesis of Compound I-9

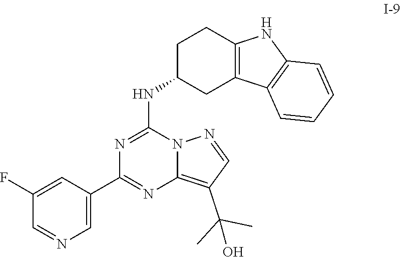 [SMILES: CC(C)(O)c1cnn2c(N[C@@H]3CCc4[nH]c5ccccc5c4C3)nc(-c3cncc(F)c3)nc12]

### Synthetic Scheme:

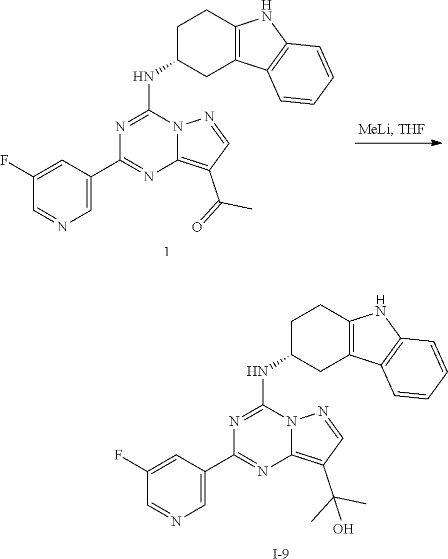

### Step 1: 2-[2-(5-Fluoro-3-pyridyl)-4-[[(3R)-2,3,4,9-tetrahydro-1H-carbazol-3-yl]amino]pyrazolo[1,5-a][1,3,5]triazin-8-yl]propan-2-ol (I-9)

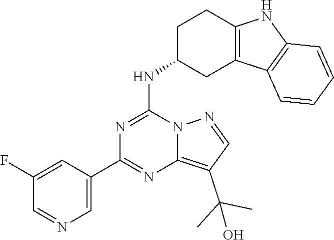 [SMILES: CC(C)(O)c1cnn2c(N[C@@H]3CCc4[nH]c5ccccc5c4C3)nc(-c3cncc(F)c3)nc12]

To MeLi (1.4 M, 10 mL, 41.20 eq) was added a solution of 1-[2-(5-fluoro-3-pyridyl)-4-[[(3R)-2,3,4,9-tetrahydro-1H-carbazol-3-yl]amino]pyrazolo[1,5-a][1,3,5]triazin-8-yl]ethanone (150 mg, 339.78 μmol, 1 eq) in THE (10 mL) dropwise under N₂ atmosphere at −30° C. Then the mixture was stirred at −5° C.˜−30° C. for 10 minutes. The reaction mixture was quenched with H₂O (10 mL) and extracted with EtOAc (15 mL×3). The organic layer was dried over Na₂SO₄, filtered and concentrated under reduced pressure to yield a residue which was purified by preparative HPLC (column: Phenomenex Gemini 150*25 mm*10 um; mobile phase: [water (0.04% NH₃.H₂O+10 mM NH₄HCO₃)−ACN]; B %: 55%-85%, 8 min). The desired fraction was lyophilized to yield 2-[2-(5-fluoro-3-pyridyl)-4-[[(3R)-2,3,4,9-tetrahydro-1H-carbazol-3-yl]amino]pyrazolo [1,5-a][1,3,5]triazin-8-yl]propan-2-ol (25.34 mg, 51.51 μmol, 15.2% yield, 93.0% purity, [α]^{23.6}_{D}=+36.238 (MeOH, c=0.100 g/100 mL)) as a white solid. ¹H NMR (400 MHz, CDCl₃) δ ppm 9.50 (s, 1H), 8.58 (d, J=2.8 Hz, 1H), 8.43-8.38 (m, 1H), 7.94-7.89 (m, 2H), 7.48 (d, J=7.6 Hz, 1H), 7.35 (d, J=8.0 Hz, 1H), 7.19 (t, J=7.2 Hz, 1H), 7.15-7.10 (m, 1H), 6.79 (d, J=8.0 Hz, 1H), 5.02-4.94 (m, 1H), 3.41-3.34 (m, 2H), 3.02-2.92 (m, 3H), 2.40-2.32 (m, 2H), 1.76 (s, 6H); ES-LCMS m/z 458.3 [M+H]⁺.

### Example 11

### Synthesis of Compound I-10

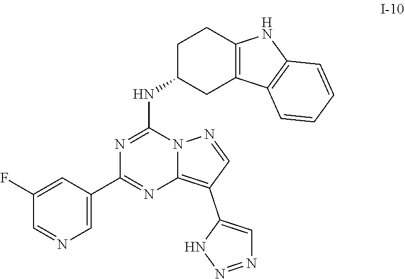 [SMILES: Fc1cncc(-c2nc(N[C@@H]3CCc4[nH]c5ccccc5c4C3)n3ncc(-c4cnn[nH]4)c3n2)c1]

### Synthetic Scheme:

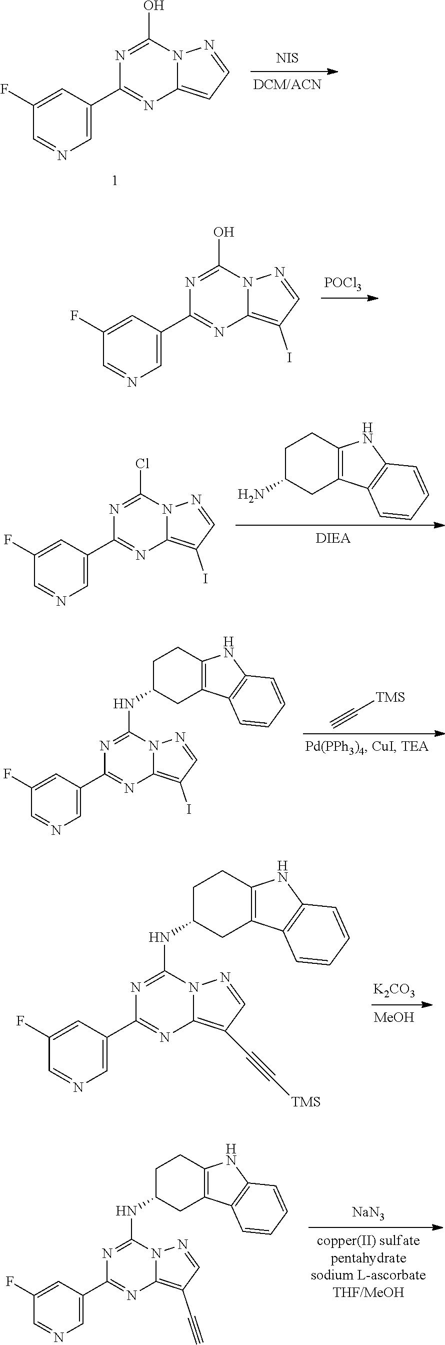

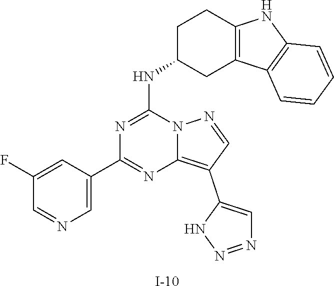

### Step 1: 2-(5-Fluoro-3-pyridyl)-8-iodo-pyrazolo[1,5-a][1,3,5]triazin-4-ol

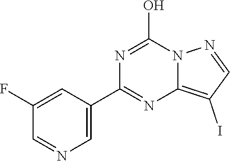 [SMILES: Oc1nc(-c2cncc(F)c2)nc2c(I)cnn12]

To a solution of 2-(5-fluoro-3-pyridyl)pyrazolo[1,5-a][1,3,5]triazin-4-ol (4.50 g, 19.46 mmol, 1 eq) in ACN (30 mL) and DCM (30 mL) was added NIS (6.57 g, 29.20 mmol, 1.5 eq). The mixture was stirred at 50° C. for 3 h under N₂ atmosphere. The reaction mixture was filtered and the cake was dried under reduced pressure to yield 2-(5-fluoro-3-pyridyl)-8-iodo-pyrazolo[1,5-a][1,3,5]triazin-4-ol (6 g, 15.96 mmol, 82.0% yield, 95.0% purity) as yellow solid which was used in the next step without further purification. ¹H NMR (400 MHz, DMSO-d₆) δ ppm 9.13 (s, 1H), 8.85 (d, J=2.7 Hz, 1H), 8.33 (br d, J=9.0 Hz, 1H), 8.23 (s, 1H); ES-LCMS m/z 358.0 [M+H]⁺.

### Step 2: 4-Chloro-2-(5-fluoro-3-pyridyl)-8-iodo-pyrazolo[1,5-a][1,3,5]triazine

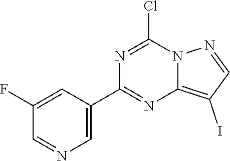 [SMILES: Fc1cncc(-c2nc(Cl)n3ncc(I)c3n2)c1]

A solution of 2-(5-fluoro-3-pyridyl)-8-iodo-pyrazolo[1,5-a][1,3,5]triazin-4-ol (5.9 g, 15.70 mmol, 1 eq) in POCl₃ (173 mL) was stirred at 130° C. for 3 h under N₂ atmosphere. LC-MS showed 40% of starting material was remained and 42% of desired compound was detected. The solution was stirred at 130° C. for 9 h under N₂ atmosphere. The mixture was concentrated and then ice water (100 mL) was added. The mixture was extracted with EtOAc (100 mL×3). The combined organic layers were dried over Na₂SO₄, filtered and concentrated to yield a residue which was purified on silica gel column chromatography (from PE/EtOAc=1/0 to 10/3, TLC: PE/EtOAc=3/1, R_{f}=0.70) to yield 4-chloro-2-(5-fluoro-3-pyridyl)-8-iodo-pyrazolo[1,5-a][1,3,5]triazine (5 g, 9.99 mmol, 63.6% yield, 75.0% purity) as a yellow solid. ¹H NMR (400 MHz, CD3OD) δ ppm 9.52 (s, 1H), 8.72 (s, 2H), 8.29 (s, 1H); ES-LCMS m/z 375.9 [M+H]⁺.

### Step 3: (3R)—N-[2-(5-Fluoro-3-pyridyl)-8-iodo-pyrazolo[1,5-a][1,3,5]triazin-4-yl]-2,3,4,9-tetrahydro-1H-carbazol-3-amine

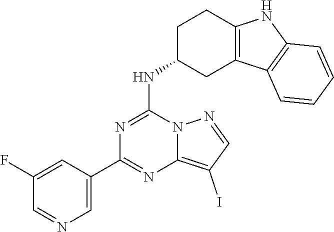 [SMILES: Fc1cncc(-c2nc(N[C@@H]3CCc4[nH]c5ccccc5c4C3)n3ncc(I)c3n2)c1]

A mixture of 4-chloro-2-(5-fluoro-3-pyridyl)-8-iodo-pyrazolo[1,5-a][1,3,5]triazine (1 g, 2.00 mmol, 1 eq), (3R)-2,3,4,9-tetrahydro-1H-carbazol-3-amine (446.38 mg, 2.40 mmol, 1.2 eq) and DIEA (774.37 mg, 5.99 mmol, 1.04 mL, 3 eq) in i-PrOH (10 mL) was degassed and purged with N₂ for 3 times. The mixture was stirred at 60° C. for 3 h under N₂ atmosphere. The reaction mixture was concentrated under reduced pressure to yield a residue which was purified on silica gel column chromatography (from PE/(EtOAc:DCM=4:1)=1/0 to 10/3, TLC: PE/EtOAc=3/1, R_{f}=0.60) to yield (3R)—N-[2-(5-fluoro-3-pyridyl)-8-iodo-pyrazolo[1,5-a][1,3,5]triazin-4-yl]-2,3,4,9-tetrahydro-1H-carbazol-3-amine (900 mg, 1.59 mmol, 79.8% yield, 93.0% purity) as a yellow solid. ¹H NMR (400 MHz, CDCl₃) δ ppm 9.54 (s, 1H), 8.60 (d, J=2.9 Hz, 1H), 8.54-8.49 (m, 1H), 8.08 (s, 1H), 7.91 (s, 1H), 7.48 (d, J=7.8 Hz, 1H), 7.36 (d, J=8.1 Hz, 1H), 7.23-7.17 (m, 1H), 7.16-7.10 (m, 1H), 6.83 (J=8.1 Hz, 1H), 4.98 (m, 1H), 3.42-3.35 (m, 1H), 3.07-2.94 (m, 3H), 2.42-2.31 (m, 2H); ES-LCMS m/z 526.1 [M+H]⁺.

### Step 4: (3R)—N-[2-(5-Fluoro-3-pyridyl)-8-(2-trimethylsilylethynyl)pyrazolo[1,5-a][1,3,5]triazin-4-yl]-2,3,4,9-tetrahydro-1H-carbazol-3-amine

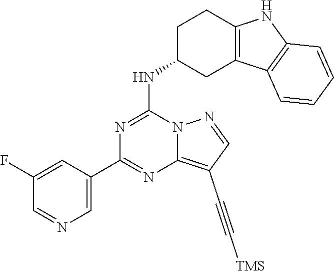 [SMILES: C[Si](C)(C)C#Cc1cnn2c(N[C@@H]3CCc4[nH]c5ccccc5c4C3)nc(-c3cncc(F)c3)nc12]

A mixture of (3R)—N-[2-(5-fluoro-3-pyridyl)-8-iodo-pyrazolo[1,5-a][1,3,5]triazin-4-yl]-2,3,4,9-tetrahydro-1H-carbazol-3-amine (150 mg, 265.55 μmol, 1 eq), CuI (10.11 mg, 53.11 μmol, 0.2 eq), Pd(PPh₃)₄ (30.69 mg, 26.56 μmol, 0.1 eq) and ethynyl(trimethyl)silane (521.65 mg, 5.31 mmol, 735.76 μL, 20 eq) in TEA (5 mL) was degassed and purged with N₂ for 3 times. The mixture was stirred at 70° C. for 5 h under N₂ atmosphere. The reaction mixture was filtered and concentrated under reduced pressure to yield a residue which was purified on silica gel column chromatography (from PE/EtOAc=1/0 to 10/3, TLC: PE/EtOAc=3/1, R_{f}=0.25) to yield (3R)—N-[2-(5-fluoro-3-pyridyl)-8-(2-trimethylsilylethynyl)pyrazolo[1,5-a][1,3,5]triazin-4-yl]-2,3,4,9-tetrahydro-1H-carbazol-3-amine (120 mg, 205.80 μmol, 77.5% yield, 85.0% purity) as a yellow solid. ¹H NMR (400 MHz, CDCl₃) δ ppm 8.53 (br s, 1H), 8.07 (s, 1H), 7.89 (s, 1H), 7.48 (J 8.0 Hz, 1H), 7.35 (d, J=7.8 Hz, 1H), 7.21-7.18 (m, 1H), 7.13 (d, J=7.8 Hz, 1H), 6.82 (s, 1H), 4.96 (m, 1H), 3.37 (m, 1H), 3.00 (m, 3H), 2.38 (m, 2H), 0.31 (s, 9H); ES-LCMS m/z 496.2 [M+H]⁺.

### Step 5: (3R)—N-[8-Ethynyl-2-(5-fluoro-3-pyridyl)pyrazolo[1,5-a][1,3,5]triazin-4-yl]-2,3,4,9-tetrahydro-1H-carbazol-3-amine

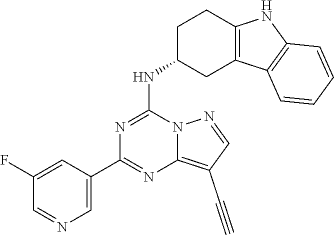 [SMILES: C#Cc1cnn2c(N[C@@H]3CCc4[nH]c5ccccc5c4C3)nc(-c3cncc(F)c3)nc12]

To a solution of (3R)—N-[2-(5-fluoro-3-pyridyl)-8-(2-trimethylsilylethynyl)pyrazolo [1,5-a][1,3,5]triazin-4-yl]-2,3,4,9-tetrahydro-1H-carbazol-3-amine (100 mg, 171.50 μmol, 1 eq) in MeOH (5 mL) was added K₂CO₃ (71.11 mg, 514.50 μmol, 3 eq). The mixture was stirred at 15° C. for 2 h under N₂ atmosphere. TLC (PE/EtOAc=3/1, R_{f}=0.25) indicated one major new spot was detected. The mixture was concentrated and then water (10 mL) was added. The mixture was extracted with EtOAc (10 mL×3). The combined organic layers were dried over Na₂SO₄, filtered and concentrated to yield the residue which was purified on silica gel column chromatography (from PE/EtOAc=1/0 to 10/3, TLC: PE/EtOAc=3/1, R_{f}=0.25) to yield (3R)—N-[8-ethynyl-2-(5-fluoro-3-pyridyl)pyrazolo[1,5-a][1,3,5]triazin-4-yl]-2,3,4,9-tetrahydro-1H-carbazol-3-amine (75 mg, 164.72 μmol, 96.1% yield, 93.0% purity) as a yellow solid. ¹H NMR (400 MHz, CDCl₃) δ ppm 9.54 (s, 1H), 8.59 (d, J=2.9 Hz, 1H), 8.52 (J 7.8 Hz, 1H), 8.08 (s, 1H), 7.89 (s, 1H), 7.48 (d, J=8.3 Hz, 1H), 7.36 (d, J=7.8 Hz, 1H), 7.20 (t, J=7.0 Hz, 1H), 7.16-7.10 (m, 1H), 6.82 (J=8.1 Hz, 1H), 4.97 (m, 1H), 3.35 (m, 2H), 3.07-2.93 (m, 3H), 2.45-2.32 (m, 2H); ES-LCMS m z 424.2 [M+H]⁺.

### Step 6: (3R)—N-[2-(5-Fluoro-3-pyridyl)-8-(1H-triazol-5-yl)pyrazolo[1,5-a][1,3,5]triazin-4-yl]-2,3,4,9-tetrahydro-1H-carbazol-3-amine (I-10)

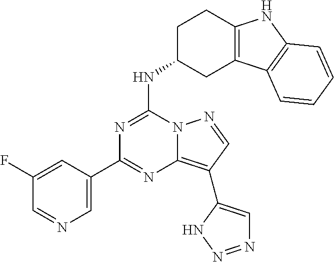 [SMILES: Fc1cncc(-c2nc(N[C@@H]3CCc4[nH]c5ccccc5c4C3)n3ncc(-c4cnn[nH]4)c3n2)c1]

A mixture of (3R)—N-[8-ethynyl-2-(5-fluoro-3-pyridyl)pyrazolo[1,5-a][1,3,5]triazin-4-yl]-2,3,4,9-tetrahydro-1H-carbazol-3-amine (75 mg, 164.72 μmol, 1 eq), sodium; (2R)-2-[(1S)-1,2-dihydroxyethyl]-4-hydroxy-5-oxo-2H-furan-3-olate (39.16 mg, 197.66 μmol, 1.2 eq), NaN₃ (12.85 mg, 197.66 umol, 1.2 eq), CuSO₄ (31.55 mg, 197.66 μmol, 1.2 eq) and (2R)-2-[(1S)-1,2-dihydroxyethyl]-3,4-dihydroxy-2H-furan-5-one (34.81 mg, 197.66 μmol, 1.2 eq) in THE (3 mL) and MeOH (3 mL) was degassed and purged with N₂ for 3 times. The mixture was stirred at 20° C. for 12 h under N₂ atmosphere. To the mixture was added DMF (2 mL), concentrated under reduced pressure to yield a residue which was purified by preparative HPLC (column: Agela ASB 150*25 mm*5 μm; mobile phase: [water (0.05% HCl)−ACN]; B %: 45%-75%, 8 min), followed by lyophilization to yield (3R)—N-[2-(5-fluoro-3-pyridyl)-8-(1H-triazol-5-yl)pyrazolo[1,5-a][1,3,5]triazin-4-yl]-2,3,4,9-tetrahydro-1H-carbazol-3-amine (24.65 mg, 42.81 μmol, 26.0% yield, 100.0% purity, 3HCl, [α]^{22.3}_{D}=+24.965 (CH₃₀H, c=0.050 g/100 mL)) as a yellow solid. ¹H NMR (400 MHz, CD3OD) δ ppm 9.66 (s, 1H), 9.17-9.76 (m, 1H), 8.94 (s, 1H), 8.61 (s, 1H), 8.56 (s, 1H), 7.36 (d, J=8.1 Hz, 1H), 7.26 (t, J=7.0 Hz, 1H), 7.04-7.00 (m, 1H), 6.96-6.92 (m, 1H), 4.98 (m, 1H), 3.31 (m, 1H), 3.07-2.91 (m, 3H), 2.44-2.38 (m, 1H), 2.31-2.27 (m, 1H); ES-LCMS m/z 424.1 [M+H]⁺.

### Example 12

### Synthesis of Compound I-11

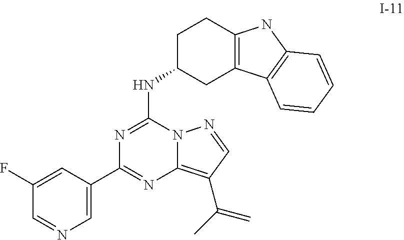 [SMILES: C=C(C)c1cnn2c(N[C@@H]3CCc4[nH]c5ccccc5c4C3)nc(-c3cncc(F)c3)nc12]

### Synthetic Scheme:

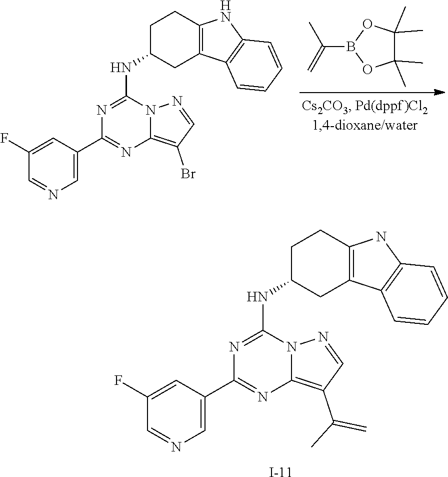

### Step 1: (3R)—N-[2-(5-Fluoro-3-pyridyl)-8-isopropenyl-pyrazolo[,5-a][1,3,5]triazin-4-yl]-2,3,4,9-tetrahydro-1H-carbazol-3-amine (I-11)

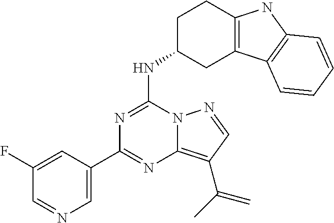 [SMILES: C=C(C)c1cnn2c(N[C@@H]3CCc4[nH]c5ccccc5c4C3)nc(-c3cncc(F)c3)nc12]

To a solution of (3R)—N-[8-bromo-2-(5-fluoro-3-pyridyl)pyrazolo[1,5-a][1,3,5]triazin-4-yl]-2,3,4,9-tetrahydro-1H-carbazol-3-amine (320 mg, 669.01 μmol, 1 eq) in 1,4-dioxane (6 mL) and water (2 mL) was added Cs₂CO₃ (653.93 mg, 2.01 mmol, 3 eq), 2-isopropenyl-4,4,5,5-tetramethyl-1,3,2-dioxaborolane (168.63 mg, 1.00 mmol, 1.5 eq) and Pd(dppf)Cl₂ (48.95 mg, 66.90 μmol, 0.1 eq) under N₂ atmosphere. The mixture was degassed and purged with N₂ for three times and stirred at 110° C. for 30 min under microwave. The reaction mixture was diluted with EtOAc (50 mL), filtered through a pad of celite, dried over Na₂SO₄ and the filtrate was concentrated to yield a residue which was purified by flash silica gel chromatography (from PE/EtOAc=100/1 to 3/1, TLC: PE/EtOAc=3/1, R_{f}=0.35) to yield (3R)—N-[2-(5-fluoro-3-pyridyl)-8-isopropenyl-pyrazolo[1,5-a][1,3,5]triazin-4-yl]-2,3,4,9-tetrahydro-1H-carbazol-3-amine (130 mg, 286.33 μmol, 42.8% yield, 96.8% purity, [α]^{24.7}_{D}=+6.152, MeOH, c=0.100 g/100 mL) as a yellow solid. ¹H NMR (400 MHz, DMSO-d₆) δ ppm 10.80 (s, 1H), 9.39 (s, 1H), 9.18 (d, J=8.8 Hz, 1H), 8.71 (d, J=2.9 Hz, 1H), 8.50-8.43 (m, 1H), 8.37 (s, 1H), 7.39-7.24 (m, 2H), 7.07-6.89 (m, 2H), 5.98 (s, 1H), 5.11 (s, 1H), 4.86 (m, 1H), 3.15-2.85 (m, 4H), 2.31-2.12 (m, 5H); ES-LCMS m/z 440.2 [M+H]⁺.

### Example 13

### Synthesis of Compound I-12

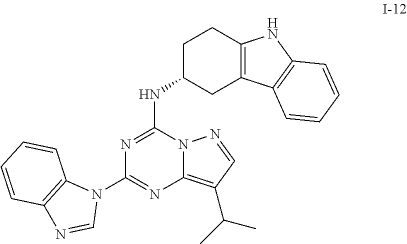 [SMILES: CC(C)c1cnn2c(N[C@@H]3CCc4[nH]c5ccccc5c4C3)nc(-n3cnc4ccccc43)nc12]

### Synthetic Scheme:

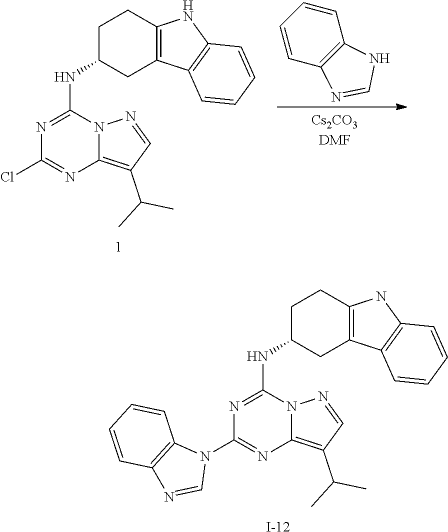

### Step 1: (3R)—N-[2-(Benzimidazol-1-yl)-8-isopropyl-pyrazolo[1,5-a][1,3,5]triazin-4-yl]-2,3,4,9-tetrahydro-1H-carbazol-3-amine (I-12)

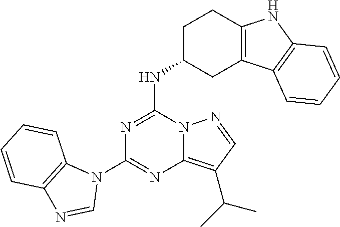 [SMILES: CC(C)c1cnn2c(N[C@@H]3CCc4[nH]c5ccccc5c4C3)nc(-n3cnc4ccccc43)nc12]

To a solution of (3R)—N-(2-chloro-8-isopropyl-pyrazolo[1,5-a][1,3,5]triazin-4-yl)-2,3,4,9-tetrahydro-1H-carbazol-3-amine (70 mg, 182.69 μmol, 1 eq) in DMF (3 mL) was added benzimidazole (215.82 mg, 1.83 mmol, 10 eq) and Cs₂CO₃ (178.57 mg, 548.06 μmol, 3 eq). The mixture was stirred at 130° C. for 1 h under microwave (1 bar). The mixture was concentrated and then EtOAc (30 mL) was added. The mixture was extracted with water (20 mL×3). The combined organic layers were washed with 5% aq. LiCi (20 mL), dried over Na₂SO₄, filtered and concentrated to yield a residue which was purified by preparative HPLC (column: Agela ASB 150*25 mm*5 μm; mobile phase: [water (0.05% HCl)−ACN]; B %: 65%-95%, 8 min), followed by lyophilization to yield (3R)—N-[2-(benzimidazol-1-yl)-8-isopropyl-pyrazolo[1,5-a][1,3,5]triazin-4-yl]-2,3,4,9-tetrahydro-1H-carbazol-3-amine (30 mg, 60.12 μmol, 32.9% yield, 100% purity, HCl, [α]^{22.3}_{D}=+31.274 (MeOH, c=0.047 g/100 mL)) as a white solid. ¹H NMR (400 MHz, CD3OD) δ ppm 9.81 (s, 1H), 8.90-8.81 (m, 1H), 8.05 (s, 1H), 7.86-7.79 (m, 1H), 7.64-7.54 (m, 2H), 7.38 (d, J=7.8 Hz, 1H), 7.28 (d, J=8.1 Hz, 1H), 7.04 (t, J=7.1 Hz, 1H), 7.00-6.91 (m, 1H), 4.84-4.80 (m, 1H), 3.35 (d, J=5.4 Hz, 1H), 3.29-3.22 (m, 1H), 3.15-2.89 (m, 3H), 2.40 (s, 1H), 2.34-2.22 (m, 1H), 1.46 (d, J=7.1 Hz, 6H); ES-LCM S m/z 463.2 [M+H]⁺.

### Example 14

### Synthesis of Compound I-13a, I-13b and I-13c

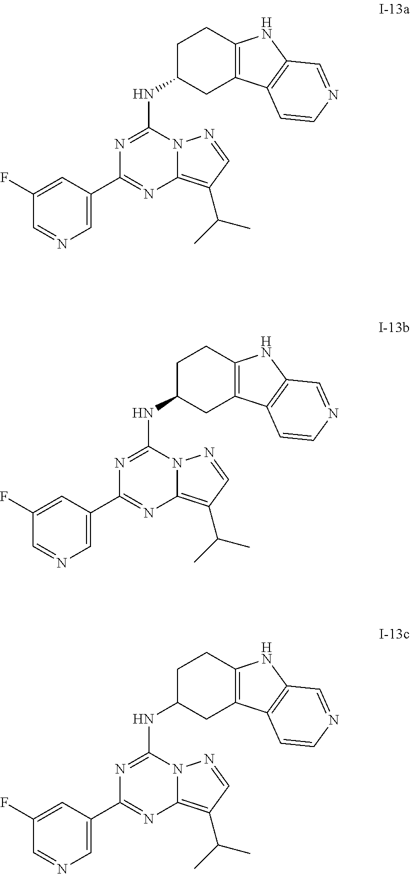 [SMILES: CC(C)c1cnn2c(N[C@@H]3CCc4[nH]c5cnccc5c4C3)nc(-c3cncc(F)c3)nc12 ; CC(C)c1cnn2c(N[C@H]3CCc4[nH]c5cnccc5c4C3)nc(-c3cncc(F)c3)nc12 ; CC(C)c1cnn2c(NC3CCc4[nH]c5cnccc5c4C3)nc(-c3cncc(F)c3)nc12]

### Synthetic Scheme:

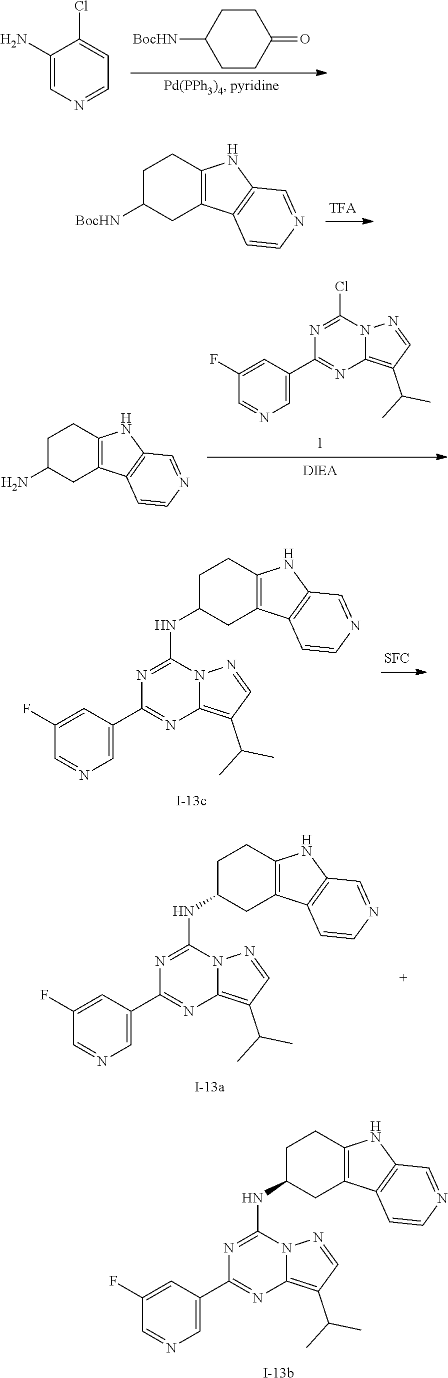

### Step 1: tert-Butyl N-(6,7,8,9-tetrahydro-5H-pyrido[3,4-b]indol-6-yl)carbamate

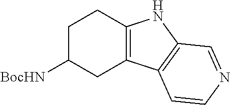 [SMILES: CC(C)(C)OC(=O)NC1CCc2[nH]c3cnccc3c2C1]

To a mixture of 4-chloropyridin-3-amine (1 g, 7.78 mmol, 1 eq) in pyridine (5 mL) was added tert-butyl N-(4-oxocyclohexyl)carbamate (1.99 g, 9.33 mmol, 1.99 mL, 1.2 eq) and Pd(PPh₃)₄ (179.77 mg, 155.57 μmol, 0.02 eq) under N₂ atmosphere. The mixture was stirred in the microwave at 160° C. for 2 h under N₂ atmosphere. The mixture was concentrated under reduced pressure to remove the solvent. The mixture was diluted with H₂O (20 mL), extracted with EtOAc (20 mL×3). The combined organic layers were washed with brine (30 mL), dried over Na₂SO₄, filtered and concentrated to yield a residue which was purified on silica gel column chromatography (from CH₂Cl₂/MeOH=100/0 to 10/1, TLC: CH₂Cl₂/MeOH=10/1, R_{f}=0.1) to yield tert-butyl N-(6,7,8,9-tetrahydro-5H-pyrido[3,4-b]indol-6-yl)carbamate (250 mg, 600.3 μmol, 7.7% yield, 69.0% purity) as a yellow solid. ¹H NMR (400 MHz, CDCl₃) δ ppm 8.64 (s, 1H), 8.07 (d, J=5.4 Hz, 1H), 7.30 (d, J=5.4 Hz, 1H), 4.69 (s, 1H), 4.03 (s, 1H), 2.90-2.82 (m, 2H), 2.39-2.22 (m, 2H), 1.97-1.85 (m, 2H), 1.40 (s, 9H); ES-LCMS m/z 288.3 [M+H]⁺.

### Step 2: 6,7,8,9-Tetrahydro-5H-pyrido[3,4-b]indol-6-amine

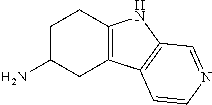 [SMILES: NC1CCc2[nH]c3cnccc3c2C1]

To a mixture of tert-butyl N-(6,7,8,9-tetrahydro-5H-pyrido[3,4-b]indol-6-yl)carbamate (200 mg, 480.24 umol, 1 eq) in DCM (6 mL) was added TFA (3.08 g, 27.01 mmol, 2 mL, 56.25 eq). The mixture was stirred at 16° C. for 1h. The reaction mixture was concentrated under reduced pressure to yield 6,7,8,9-tetrahydro-5H-pyrido[3,4-b]indol-6-amine (200 mg, 464.71 μmol, 96.8% yield, 70.0% purity, TFA) as a yellow solid which was used in next step without further purification. ¹H NMR (400 MHz, CDCl₃) δ ppm 8.88 (s, 1H), 8.16 (d, J=6.4 Hz, 1H), 7.93 (d, J=6.6 Hz, 1H), 3.78-3.70 (m, 1H), 3.19-3.13 (m, 2H), 2.86 (dd, J=9.0, 15.4 Hz, 1H), 2.41-2.35 (m, 1H), 2.17-2.06 (m, 2H); ES-LCMS m/z 188.0 [M+H]⁺.

### Step 3: (6R)—N-[2-(5-fluoro-3-pyridyl)-8-isopropyl-pyrazolo[1,5-a][1,3,5]triazin-4-yl]-6,7,8,9-tetrahydro-5H-pyrido[3,4-b]indol-6-amine (I-13a) and (6S)—N-[2-(5-fluoro-3-pyridyl)-8-isopropyl-pyrazolo[1,5-a][1,3,5]triazin-4-yl]-6,7,8,9-tetrahydro-5H-pyrido[3,4-b]indol-6-amine (I-13b)

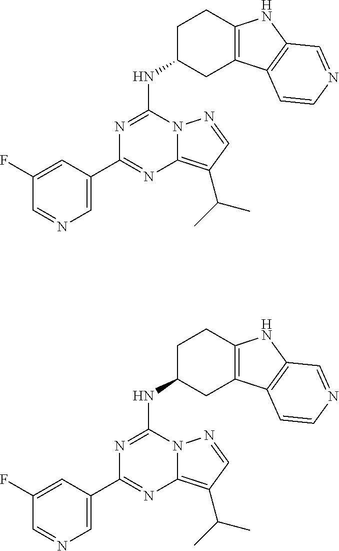 [SMILES: CC(C)c1cnn2c(N[C@@H]3CCc4[nH]c5cnccc5c4C3)nc(-c3cncc(F)c3)nc12 ; CC(C)c1cnn2c(N[C@H]3CCc4[nH]c5cnccc5c4C3)nc(-c3cncc(F)c3)nc12]

To a mixture of 6,7,8,9-tetrahydro-5H-pyrido[3,4-b]indol-6-amine (200 mg, 747.70 μmol, 1 eq) in i-PrOH (10 mL) was added 4-chloro-2-(5-fluoro-3-pyridyl)-8-isopropyl-pyrazolo [1,5-a][1,3,5]triazine (197.16 mg, 598.16 μmol, 0.8 eq) and DIEA (483.18 mg, 3.74 mmol, 651.18 μL, 5 eq). The mixture was stirred at 60° C. for 3 h. The reaction mixture was filtered and concentrated under reduced pressure to yield a residue which was purified by preparative HPLC (HCl condition; column: Xtimate C18 150*25 mm*5 um; mobile phase: [water (0.05% HCl)−ACN]; B %: 45%-75%, 8 min and SFC column: DAICEL CHIRALPAK AD-H (250 mm*30 mm, 5 um); mobile phase: [0.1% NH₃H₂O IPA]; B %: 30%-30%). The desired fraction was concentrated under reduced and lyophilized to yield an enantiomer (12.89 mg, 28.43 umol, 3.8% yield, 97.6% purity, SFC: Rt=6.022, ee=99.400%, [α]^{23.2}_{D}=+20.575 (MeOH, c=0.020 g/100 mL)) as a white solid. ¹H NMR (400 MHz, CD3OD) δ ppm 9.37 (s, 1H), 8.54 (s, 1H), 8.51 (d, J=2.7 Hz, 1H), 8.49-8.45 (m, 1H), 8.00 (d, J=5.6 Hz, 1H), 7.96 (s, 1H), 7.44 (d, J=5.1 Hz, 1H), 4.86-4.86 (m, 1H), 3.37-3.31 (m, 1H), 3.27-3.17 (m, 1H), 3.14-2.89 (m, 3H), 2.42 (d, J=12.2 Hz, 1H), 2.32-2.21 (m, 1H), 1.39 (d, J=7.1 Hz, 6H); ES-LCMS m/z 443.3 [M+H]⁺; and the other enantiomer (16.05 mg, 35.35 umol, 4.7% yield, 97.5% purity, SFC: Rt=6.671, ee=98.746%, [α]^{23.2}_{D}=−25.506 (MeOH, c=0.020 g/100 mL)) as a white solid. ¹H NMR (400 MHz, CD3OD) δ ppm 9.36 (s, 1H), 8.54 (s, 1H), 8.50 (d, J=2.7 Hz, 1H), 8.45 (d, J=9.8 Hz, 1H), 8.00 (d, J=5.6 Hz, 1H), 7.95 (s, 1H), 7.45 (d, J=5.6 Hz, 1H), 4.87-4.87 (m, 1H), 3.34 (d, J=5.4 Hz, 1H), 3.28-3.21 (m, 1H), 3.15-2.88 (m, 3H), 2.41 (d, J=13.2 Hz, 1H), 2.33-2.19 (m, 1H), 1.39 (d, J=7.1 Hz, 6H); ES-LCMS m/z 443.3 [M+H]⁺.

### Example 15

### Synthesis of Compound I-14

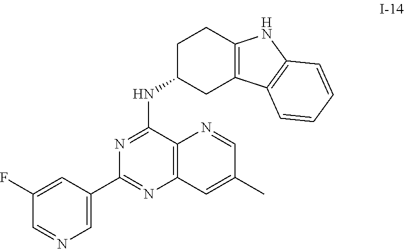 [SMILES: Cc1cnc2c(N[C@@H]3CCc4[nH]c5ccccc5c4C3)nc(-c3cncc(F)c3)nc2c1]

### Synthetic Scheme:

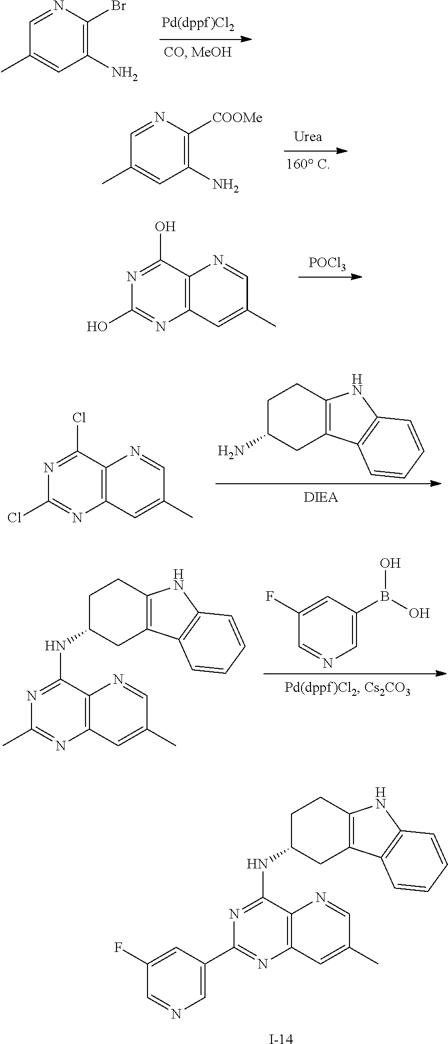

### Step 1: Methyl 3-amino-5-methyl-pyridine-2-carboxylate

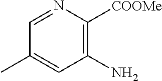

To a solution of 2-bromo-5-methyl-pyridin-3-amine (3 g, 16.04 mmol, 1 eq) in MeOH (10 mL) was added Pd(dppf)Cl₂ (1.17 g, 1.60 mmol, 0.1 eq) and TEA (1.62 g, 16.04 mmol, 2.23 mL, 1 eq). The suspension was degassed under vacuum and purged with CO several times. The mixture was stirred under CO (50 psi) at 50° C. for 48 hr. The mixture was filtered and concentrated under reduced pressure to yield a residue which was purified by column chromatography (SiO₂, From PE/EtOAc=10/1 to 1/1, TLC: PE/EtOAc=1/1, R_{f}=0.53) to yield methyl 3-amino-5-methyl-pyridine-2-carboxylate (1.5 g, crude) as a brown solid. ¹H NMR (400 MHz, CDCl₃) δ ppm 7.90 (s, 1H), 6.84 (s, 1H), 3.93 (s, 3H), 2.29 (s, 3H); ES-LCMS m/z 167.1 [M+H]⁺.

### Step 2: 7-Methylpyrido[3,2-d]pyrimidine-2,4-diol

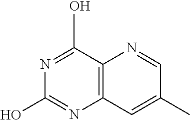

A mixture of methyl 3-amino-5-methyl-pyridine-2-carboxylate (1.5 g, 9.03 mmol, 1 eq) and urea (2.71 g, 45.13 mmol, 2.42 mL, 5 eq) was stirred at 160° C. for 12 hr. The mixture was diluted with water (20 mL) and EtOH (30 mL), stirred at 20° C. for 30 min, then filtered and washed with water (20 mL) and EtOH (30 mL×3) to yield 7-methylpyrido[3,2-d]pyrimidine-2,4-diol (381 mg, 1.94 mmol, 21.44% yield, 90% purity) as a gray solid which was used in the next step without further purification. ¹H NMR (400 MHz, DMSO-d₆) δ ppm 11.18 (br s, 2H), 8.31 (s, 1H), 7.33 (s, 1H), 2.38 (s, 3H); ES-LCMS m/z 178.0 [M+H]⁺.

### Step 3: 2,4-Dichloro-7-methyl-pyrido[3,2-d]pyrimidine

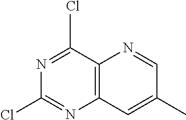

A mixture of 7-methylpyrido[3,2-d]pyrimidine-2,4-diol (150 mg, 762.02 μmol, 1 eq), DIEA (984.86 mg, 7.62 mmol, 1.33 mL, 10 eq) and POCl₃ (1.17 g, 7.62 mmol, 708.14 μL, 10 eq) in toluene (10 mL) was stirred at 110° C. for 2 hr. The mixture was concentrated under reduced pressure to yield 2,4-dichloro-7-methyl-pyrido[3,2-d]pyrimidine (170 mg, crude) as a black solid which was used in the next step without further purification. ¹H NMR (400 MHz, CDCl₃) δ ppm 8.98 (s, 1H), 8.07 (s, 1H), 2.70 (s, 3H); ES-LCMS m/z 213.9 [M+H]⁺.

### Step 4: (3R)—N-(2-chloro-7-methyl-pyrido[3,2-d]pyrimidin-4-yl)-2,3,4,9-tetrahydro-1H-carbazol-3-amine

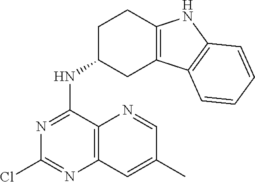 [SMILES: Cc1cnc2c(N[C@@H]3CCc4[nH]c5ccccc5c4C3)nc(Cl)nc2c1]

A mixture of 2,4-dichloro-7-methyl-pyrido[3,2-d]pyrimidine (90 mg, 420.46 μmol, 1 eq), (3R)-2,3,4,9-tetrahydro-1H-carbazol-3-amine (86.14 mg, 462.51 μmol, 1.1 eq), and DIEA (54.34 mg, 420.46 μmol, 73.24 μL, 1 eq) in MeCN (10 mL) was stirred at 70° C. for 1 hr. The mixture was concentrated under reduced pressure to yield a residue which was purified by column chromatography (SiO₂, From PE/EtOAc=10/1 to 1/1, TLC: PE/EtOAc=1/1, R_{f}=0.68) to yield (3R)—N-(2-chloro-7-methyl-pyrido[3,2-d]pyrimidin-4-yl)-2,3,4,9-tetrahydro-1H-carbazol-3-amine (74 mg, 183.05 μmol, 43.53% yield, 90% purity) as a yellow solid. ¹H NMR (400 MHz, CDCl₃) δ ppm 8.45 (d, J=2.0 Hz, 1H), 7.85 (s, 1H), 7.77 (s, 1H), 7.49 (d, J=8.4 Hz, 1H), 7.41 (d, J=8.4 Hz, 1H), 7.34 (d, J=8.0 Hz, 1H), 7.22-7.08 (m, 2H), 4.94-4.83 (m, 1H), 3.34-3.28 (m, 1H), 3.04-2.83 (m, 3H), 2.51 (s, 3H), 2.33-2.24 (m, 2H); ES-LCMS m/z 364.1 [M+H]⁺.

### Step 5: (3R)—N-[2-(5-fluoro-3-pyridyl)-7-methyl-pyrido[3,2-d]pyrimidin-4-yl]-2,3,4,9-tetrahydro-1H-carbazol-3-amine (I-14)

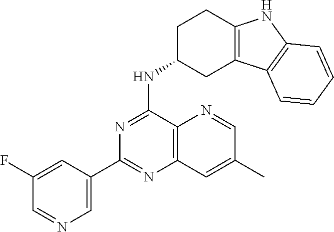 [SMILES: Cc1cnc2c(N[C@@H]3CCc4[nH]c5ccccc5c4C3)nc(-c3cncc(F)c3)nc2c1]

A mixture of (3R)—N-(2-chloro-7-methyl-pyrido[3,2-d]pyrimidin-4-yl)-2,3,4,9-tetrahydro-1H-carbazol-3-amine (74 mg, 183.05 μmol, 1 eq), (5-fluoro-3-pyridyl)boronic acid (30.95 mg, 219.66 μmol, 1.2 eq), Pd(dppf)Cl₂ (13.39 mg, 18.30 μmol, 0.1 eq) and Cs₂CO₃ (119.28 mg, 366.09 μmol, 2 eq) in 1,4-dioxane (2 mL) and H₂O (0.5 mL) was stirred at 100° C. for 0.5 hr under microwave (1 bar). The mixture was concentrated under reduced pressure to yield a residue which was purified by preparative-HPLC (column: Xtimate C18 150*25 mm*5 um; mobile phase: [water (0.05% HCl)−ACN]; B %: 50%-80%, 8 min) to yield (3R)—N-[2-(5-fluoro-3-pyridyl)-7-methyl-pyrido[3,2-d]pyrimidin-4-yl]-2,3,4,9-tetrahydro-1H-carbazol-3-amine (17.82 mg, 41.41 μmol, 22.6% yield, 98.6% purity, [α]^{23.3}_{D}=+13.629 (MeOH, c=0.071 g/100 mL)) as a brown solid. ¹H NMR (400 MHz, CD3OD) δ ppm 9.34 (s, 1H), 8.83 (d, J=1.6 Hz, 1H), 8.77 (d, J=2.4 Hz, 1H), 8.57-8.48 (m, 1H), 8.05 (s, 1H), 7.38 (d, J=7.6 Hz, 1H), 7.29 (d, J=8.0 Hz, 1H), 7.05 (t, J=7.2 Hz, 1H), 7.01-6.92 (m, 1H), 5.18-5.03 (m, 1H), 3.31 (m, 1H), 3.04-2.95 (m, 3H), 2.64 (s, 3H), 2.45-2.29 (m, 2H); ES-LCMS m/z 425.2 [M+H]⁺.

### Example 16

### Synthesis of Compound I-15a, I-15b and I-15c

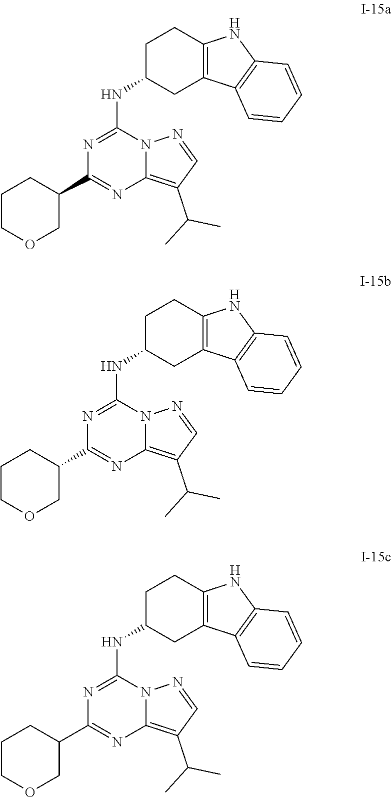

### Synthetic Scheme:

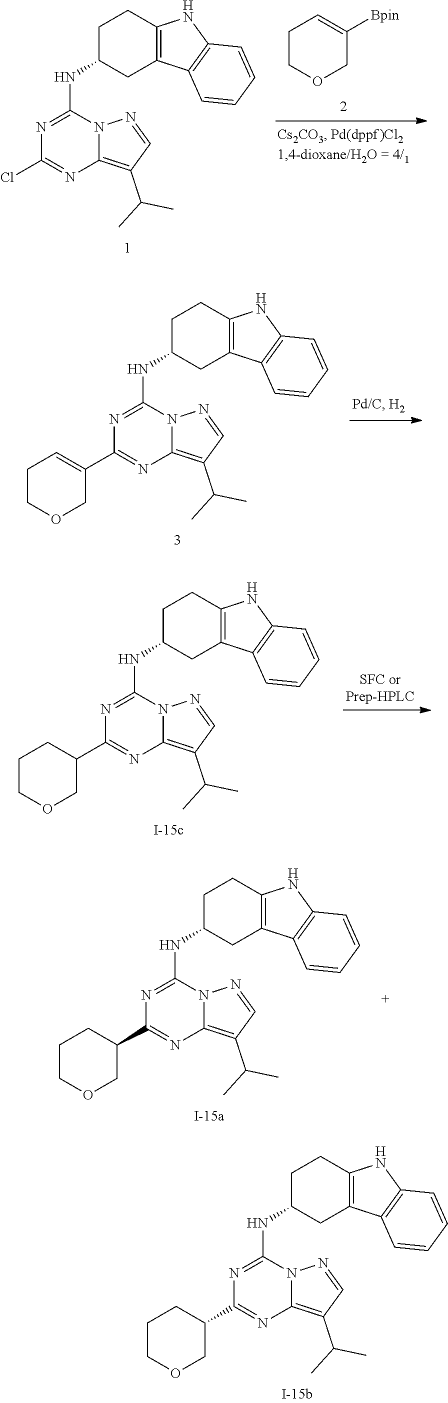

### Step 1: (3R)—N-[2-(3,6-dihydro-2H-pyran-5-yl)-8-isopropyl-pyrazolo[1,5-a][1,3,5]triazin-4-yl]-2,3,4,9-tetrahydro-1H-carbazol-3-amine

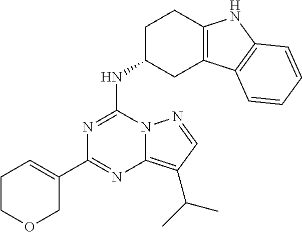 [SMILES: CC(C)c1cnn2c(N[C@@H]3CCc4[nH]c5ccccc5c4C3)nc(C3=CCCOC3)nc12]

To a suspension of (3R)—N-(2-chloro-8-isopropyl-pyrazolo[1,5-a][1,3,5]triazin-4-yl)-2,3,4,9-tetrahydro-1H-carbazol-3-amine (100 mg, 258.88 μmol, 1 eq), Pd(dppf)Cl₂ (18.94 mg, 25.89 μmol, 0.1 eq) and Cs₂CO₃ (253.04 mg, 776.64 μmol, 3 eq) in 1,4-dioxane (2 mL) and H₂O (0.5 mL) was added 2-(5,6-dihydro-2H-pyran-3-yl)-4,4,5,5-tetramethyl-1,3,2-dioxaborolane (108.77 mg, 517.76 μmol, 2 eq). The mixture was stirred under microwave at 110° C. for 0.5 h. The mixture was concentrated and water (10 mL) was added, extracted with EtOAc (30 mL×2). The combined organic layers were washed with brine (20 mL), dried over Na₂SO₄, filtered and concentrated to yield a residue which was purified by silica gel column chromatography (from pure PE to PE/EtOAc=3/1, TLC: PE/EtOAc=3/1, R_{f}=0.45) to yield (3R)—N-[2-(3,6-dihydro-2H-pyran-5-yl)-8-isopropyl-pyrazolo[1,5-a][1,3,5]triazin-4-yl]-2,3,4,9-tetrahydro-1H-carbazol-3-amine (90 mg, 189.02 μmol, 73.0% yield, 90.0% purity) as a yellow solid. ¹H NMR (400 MHz, CDCl₃) δ ppm 7.83 (s, 1H), 7.75 (s, 1H), 7.46 (d, J=7.8 Hz, 1H), 7.36-7.30 (m, 2H), 7.19-7.14 (m, 1H), 7.13-7.07 (m, 1H), 6.51 (d, J=8.1 Hz, 1H), 4.81 (d, J=8.1 Hz, 1H), 4.70 (d, J=2.0 Hz, 2H), 3.84 (t, J=5.5 Hz, 2H), 3.32 (dd, J=5.3, 15.5 Hz, 1H), 3.18 (td, J=7.0, 13.9 Hz, 1H), 2.99-2.91 (m, 2H), 2.86 (dd, J=7.3, 15.7 Hz, 1H), 2.39 (d, J=4.2 Hz, 2H), 2.35-2.22 (m, 2H), 1.34 (d, J=7.1 Hz, 6H); ES-LCMS m/z 429.2 [M+H]⁺.

### Step 2: (3R)—N-(8-isopropyl-2-tetrahydropyran-3-yl-pyrazolo[1,5-a][1,3,5]triazin-4-yl)-2,3,4,9-tetrahydro-1H-carbazol-3-amine (I-15a) and (3R)—N-[8-isopropyl-2-[(3R)-tetrahydropyran-3-yl]pyrazolo[1,5-a][1,3,5]triazin-4-yl]-2,3,4,9-tetrahydro-1H-carbazol-3-amine (I-15b)

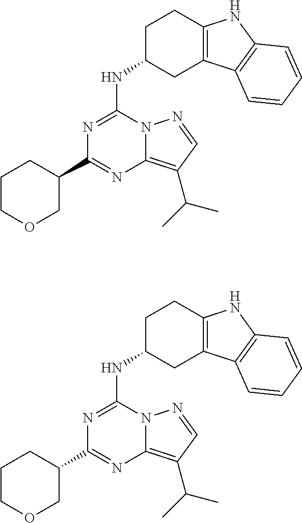 [SMILES: CC(C)c1cnn2c(N[C@@H]3CCc4[nH]c5ccccc5c4C3)nc([C@@H]3CCCOC3)nc12 ; CC(C)c1cnn2c(N[C@@H]3CCc4[nH]c5ccccc5c4C3)nc([C@H]3CCCOC3)nc12]

To a solution of (3R)—N-[2-(3,6-dihydro-2H-pyran-5-yl)-8-isopropyl-pyrazolo[1,5-a][1,3,5]triazin-4-yl]-2,3,4,9-tetrahydro-1H-carbazol-3-amine (70 mg, 147.01 μmol, 1 eq) in MeOH (3 mL) and EtOAc (3 mL) was added Pd/C (40 mg, 147.01 μmol, 10% purity, 1.00 eq). The suspension was degassed under vacuum and purged with H2 several times. The mixture was stirred under H2 (15 psi) at 20° C. for 1 h. The mixture was filtered. The filtrate was concentrated. The residue was further separated by SFC (column: DAICEL CHIRALPAK AD (250 mm*30 mm, 10 m); mobile phase: [0.1% NH₃.H₂O EtOH]; B %: 45%-45%) to yield Peak 1 and Peak 2. One of these peaks was concentrated under reduced pressure to yield a residue which was dissolved in MeCN (30 mL) and H₂O (10 mL) then lyophilized to yield an enantiomer (20.33 mg, 45.85 μmol, 18.3% yield, 97.1% purity. SFC: Rₜ=1.734 min, ee=100%. [α]^{24.5}_{D}=−7.141, THF, c=0.080 g/100 mL) as a white solid. ¹H NMR (400 MHz, CDCl₃) δ ppm 7.82 (s, 1H), 7.76 (s, 1H), 7.45 (d, J=7.6 Hz, 1H), 7.32 (d, J=7.8 Hz, 1H), 7.19-7.14 (m, 1H), 7.13-7.07 (m, 1H), 6.51 (d, J=7.8 Hz, 1H), 4.77 (s, 1H), 4.23 (d, J=8.6 Hz, 1H), 3.96 (d, J=11.0 Hz, 1H), 3.73 (t, J=10.6 Hz, 1H), 3.55-3.43 (m, 1H), 3.30 (dd, J=5.1, 15.2 Hz, 1H), 3.20 (q, J=6.8 Hz, 1H), 3.05-2.82 (m, 4H), 2.35-2.18 (m, 3H), 1.99-1.87 (m, 1H), 1.80-1.70 (m, 2H), 1.33 (d, J=6.8 Hz, 6H); ES-LCMS m/z 431.3 [M+H]⁺. The other of these peaks was concentrated under reduced pressure to yield a residue which was dissolved in MeCN (30 mL) and H₂O (10 mL) then lyophilized to yield the other enantiomer (22.05 mg, 51.05 μmol, 20.4% yield, 99.7% purity. SFC: Rₜ=2.283 min, ee=100%. [α]^{24.6}_{D}=−22.626, THF, c=0.082 g/100 mL) as a white solid. ¹H NMR (400 MHz, CDCl₃) δ ppm 7.83 (s, 1H), 7.76 (s, 1H), 7.45 (d, J=7.8 Hz, 1H), 7.32 (d, J=7.8 Hz, 1H), 7.19-7.14 (m, 1H), 7.14-7.07 (m, 1H), 6.51 (d, J=8.1 Hz, 1H), 4.78 (s, 1H), 4.28-4.20 (m, 1H), 3.97 (d, J=11.5 Hz, 1H), 3.73 (t, J=10.8 Hz, 1H), 3.48 (dt, J=3.5, 10.8 Hz, 1H), 3.30 (dd, J=5.3, 15.3 Hz, 1H), 3.20 (q, J=7.0 Hz, 1H), 3.04-2.84 (m, 4H), 2.32-2.17 (m, 3H), 1.98-1.86 (m, 1H), 1.82-1.71 (m, 2H), 1.33 (d, J=7.1 Hz, 6H); ES-LCMS m/z 431.3 [M+H]⁺.

### Example 17

### Synthesis of Compound I-16

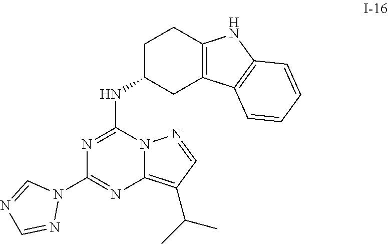 [SMILES: CC(C)c1cnn2c(N[C@@H]3CCc4[nH]c5ccccc5c4C3)nc(-n3cncn3)nc12]

### Synthetic Scheme:

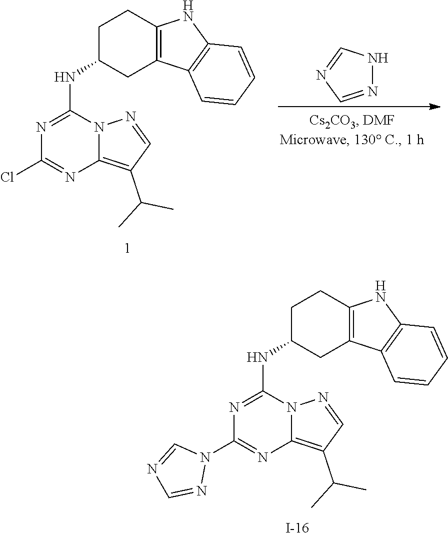

### Step 1: (3R)—N-[8-Isopropyl-2-(1,2,4-triazol-1-yl)pyrazolo[1,5-a][1,3,5]triazin-4-yl]-2,3,4,9-tetrahydro-1H-carbazol-3-amine (I-16)

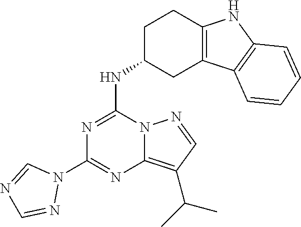 [SMILES: CC(C)c1cnn2c(N[C@@H]3CCc4[nH]c5ccccc5c4C3)nc(-n3cncn3)nc12]

To a stirred solution of (3R)—N-(2-chloro-8-isopropyl-pyrazolo[1,5-a][1,3,5]triazin-4-yl)-2,3,4,9-tetrahydro-1H-carbazol-3-amine (70 mg, 182.69 μmol, 1 eq) in DMF (3 mL) was added Cs₂CO₃ (178.57 mg, 548.06 μmol, 3 eq) and 1H-1,2,4-triazole (126.17 mg, 1.83 mmol, 10 eq). The reaction mixture was stirred at 130° C. for 1 h under microwave. The reaction mixture was filtered and the filtrate was adjusted pH to 7-8 by 1 N aq. HCl solution and diluted with MeOH (2 mL) to purify by preparative HPLC (HCl condition; column: Agela ASB 150*25 mm*5 um; mobile phase: [water (0.05% HCl)−ACN]; B %: 58%-78%, 8 min) to yield a residue which was re-purified by preparative HPLC (HCl condition; column: Agela ASB 150*25 mm*5 um; mobile phase: [water (0.05% HCl)−ACN]; B %: 58%-78%, 8 min) to remove residual DMSO. The desired fraction was lyophilized to yield (3R)—N-[8-isopropyl-2-(1,2,4-triazol-1-yl)pyrazolo[1,5-a][1,3,5]triazin-4-yl]-2,3,4,9-tetrahydro-1H-carbazol-3-amine (24.31 mg, 52.79 μmol, 28.9% yield, 97.7% purity, HCl, [α]^{25.9}_{D}=+15.468, MeOH, c=0.053 g/100 mL) as a white solid. ¹H NMR (400 MHz, CD3OD) δ ppm 9.42 (s, 1H), 8.25 (s, 1H), 8.02 (s, 1H), 7.37 (d, J=7.8 Hz, 1H), 7.26 (d, J=8.1 Hz, 1H), 7.03 (t, J=7.5 Hz, 1H), 6.98-6.92 (m, 1H), 4.84 (s, 1H), 3.30-3.23 (m, 2H), 3.13-3.01 (m, 1H), 2.99-2.87 (m, 2H), 2.40-2.17 (m, 2H), 1.37 (d, J=6.8 Hz, 6H); ES-LCMS m/z 414.2 [M+H]⁺.

### Example 18

### Synthesis of Compound I-17

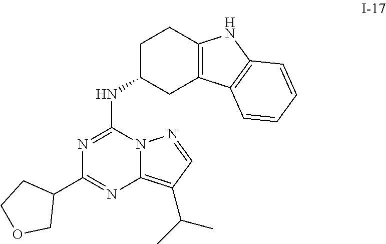 [SMILES: CC(C)c1cnn2c(N[C@@H]3CCc4[nH]c5ccccc5c4C3)nc(C3CCOC3)nc12]

### Synthetic Scheme:

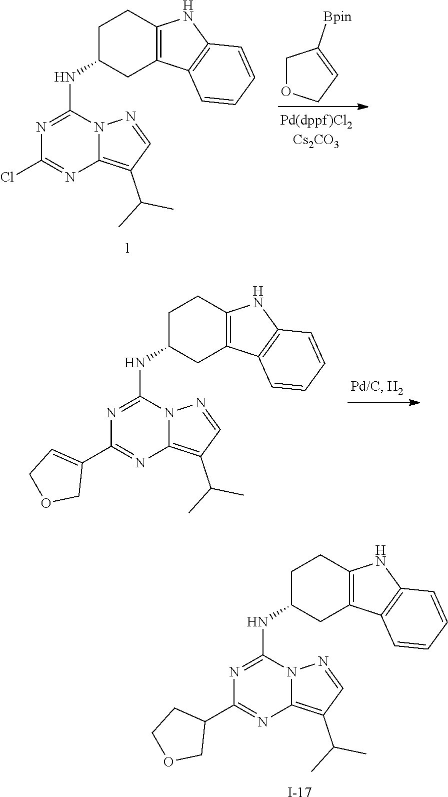

### Step 1: (3R)—N-[2-(2,5-dihydrofuran-3-yl)-8-isopropyl-pyrazolo[1,5-a][1,3,5]triazin-4-yl]-2,3,4,9-tetrahydro-1H-carbazol-3-amine

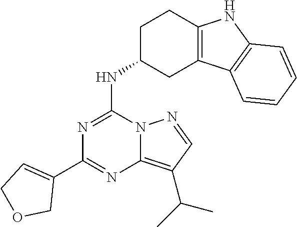 [SMILES: CC(C)c1cnn2c(N[C@@H]3CCc4[nH]c5ccccc5c4C3)nc(C3=CCOC3)nc12]

(3R)—N-(2-chloro-8-isopropyl-pyrazolo[1,5-a][1,3,5]triazin-4-yl)-2,3,4,9-tetrahydro-1H-carbazol-3-amine (200 mg, 517.76 μmol, 1 eq), 2-(2,5-dihydrofuran-3-yl)-4,4,5,5-tetramethyl-1,3,2-dioxaborolane (203.01 mg, 1.04 mmol, 2 eq), Pd(dppf)Cl₂ (37.88 mg, 51.78 μmol, 0.1 eq) and Cs₂CO₃ (506.09 mg, 1.55 mmol, 3 eq) in 1,4-dioxane (3 mL) and H₂O (1 mL) were taken up into a microwave tube and then purged with N₂ for 1 min. The sealed tube was heated at 110° C. for 1 h under microwave (1 bar). The reaction mixture was concentrated to yield a residue which was purified by flash silica gel chromatography (from pure PE to PE/EtOAc=3/1, TLC: PE/EtOAc=3/1, R_{f}=0.40) to yield (3R)—N-[2-(2,5-dihydrofuran-3-yl)-8-isopropyl-pyrazolo[1,5-a][1,3,5]triazin-4-yl]-2,3,4,9-tetrahydro-1H-carbazol-3-amine (90 mg, 214.31 μmol, 41.4% yield, 98.7% purity) as a yellow solid. ¹H NMR (400 MHz, CDCl₃) δ ppm 7.84 (s, 1H), 7.78 (s, 1H), 7.46 (d, J=7.8 Hz, 1H), 7.33 (d, J=8.0 Hz, 1H), 7.20-7.15 (m, 1H), 7.14-7.08 (m, 1H), 6.93 (t, J=2.0 Hz, 1H), 6.54 (d, J=8.3 Hz, 1H), 5.10 (dt, J=2.1, 5.0 Hz, 2H), 4.91 (dt, J=1.9, 5.0 Hz, 2H), 4.85-4.76 (m, 1H), 3.33 (dd, J=5.0, 15.6 Hz, 1H), 3.20 (td, J=7.0, 13.9 Hz, 1H), 2.99-2.84 (m, 3H), 2.37-2.21 (m, 2H), 1.34 (d, J=7.0 Hz, 6H); ES-LCMS m/z 415.2 [M+H]⁺.

### Step 2: (3R)—N-(8-isopropyl-2-tetrahydrofuran-3-yl-pyrazolo[1,5-a][1,3,5]triazin-4-yl)-2,3,4,9-tetrahydro-1H-carbazol-3-amine (I-17)

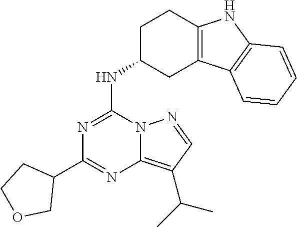 [SMILES: CC(C)c1cnn2c(N[C@@H]3CCc4[nH]c5ccccc5c4C3)nc(C3CCOC3)nc12]

To a mixture of (3R)—N-[2-(2,5-dihydrofuran-3-yl)-8-isopropyl-pyrazolo[1,5-a][1,3,5]triazin-4-yl]-2,3,4,9-tetrahydro-1H-carbazol-3-amine (80 mg, 190.49 μmol, 1 eq) and NH₃.H₂O (23.85 mg, 190.49 μmol, 26.20 μL, 28% purity, 1 eq) in MeOH (10 mL) and EtOAc (10 mL) was added Pd/C (120 mg, 10% purity). The reaction mixture was stirred at 25° C. under H2 atmosphere (15 psi) for 1 h. The reaction mixture was filtered and concentrated. The residue was purified by preparative HPLC (column: Agela ASB 150*25 mm*5 μm; mobile phase: [water (0.05% HCl)−ACN]; B %: 55%-85%, 8 min), followed by lyophilization to yield (3R)—N-(8-isopropyl-2-tetrahydrofuran-3-yl-pyrazolo[1,5-a][1,3,5] triazin-4-yl)-2,3,4,9-tetrahydro-1H-carbazol-3-amine (29.14 mg, 63.50 μmol, 33.3% yield, 98.7% purity, HCl, [α]^{27.0}_{D}=+14.485 (MeOH, c=0.095 g/100 mL)) as a white solid. ¹H NMR (400 MHz, DMSO-d₆) δ ppm 10.79 (s, 1H), 8.97 (d, J=7.3 Hz, 1H), 8.08 (s, 1H), 7.34 (d, J=7.8 Hz, 1H), 7.27 (d, J=8.0 Hz, 1H), 7.04-6.99 (m, 1H), 6.97-6.90 (m, 1H), 4.58 (dd, J=4.9, 8.7 Hz, 1H), 4.07-4.02 (m, 1H), 3.94-3.73 (m, 3H), 3.49-3.45 (m, 1H), 3.19-3.01 (m, 2H), 2.95-2.82 (m, 3H), 2.35-2.25 (m, 1H), 2.24-2.12 (m, 3H), 1.30 (d, J=6.8 Hz, 6H); ES-LCMS m/z 417.2 [M+H]⁺.

### Example 19

### Synthesis of Compound I-18

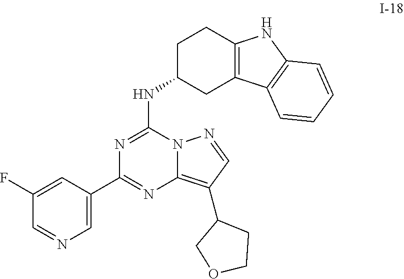 [SMILES: Fc1cncc(-c2nc(N[C@@H]3CCc4[nH]c5ccccc5c4C3)n3ncc(C4CCOC4)c3n2)c1]

### Synthetic Scheme:

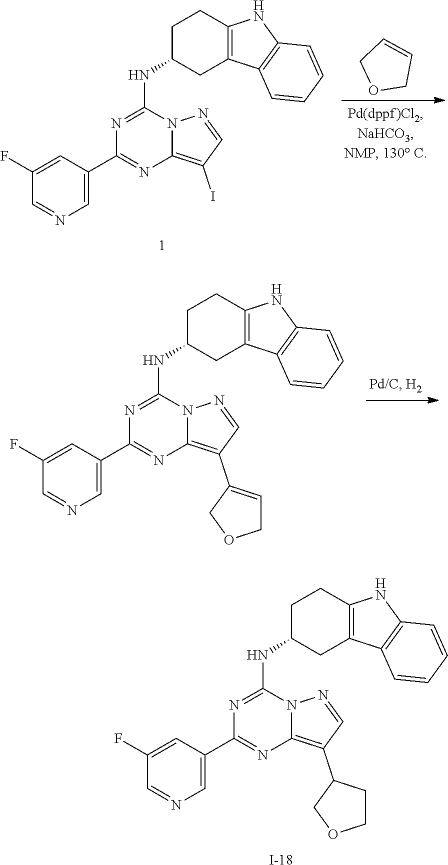

### Step 1: (3R)—N-[8-(2,5-dihydrofuran-3-yl)-2-(5-fluoro-3-pyridyl)pyrazolo[1,5-a][1,3,5]triazin-4-yl]-2,3,4,9-tetrahydro-1H-carbazol-3-amine

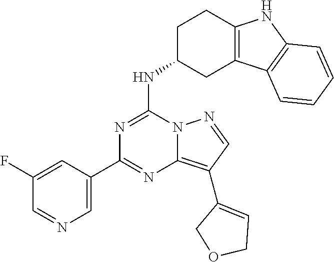 [SMILES: Fc1cncc(-c2nc(N[C@@H]3CCc4[nH]c5ccccc5c4C3)n3ncc(C4=CCOC4)c3n2)c1]

A mixture of (3R)—N-[2-(5-fluoro-3-pyridyl)-8-iodo-pyrazolo[1,5-a][1,3,5]triazin-4-yl]-2,3,4,9-tetrahydro-1H-carbazol-3-amine (150 mg, 265.55 umol, 1 eq), 2,5-dihydrofuran (18.61 mg, 265.55 μmol, 20.08 μL, 1 eq), Pd(PPh₃)₂C12 (18.64 mg, 26.56 umol, 0.1 eq), NaHCO₃ (37.93 mg, 451.44 μmol, 17.56 μL, 1.7 eq) in NMP (1 mL) was stirred at 130° C. for 2 hr under N₂. (1 bar). The mixture was filtered and concentrated under reduced pressure to yield a residue which was diluted with water (50 mL), extracted with EtOAc (50 mL×3), washed with brine (50 mL), dried over Na₂SO₄, filtered and concentrated under reduced pressure to yield a residue which was purified by column chromatography (SiO₂, From PE/EtOAc=10/1 to 1/1, TLC: PE/EtOAc=3/1, R_{f}=0.46) to yield (3R)—N-[8-(2,5-dihydrofuran-3-yl)-2-(5-fluoro-3-pyridyl)pyrazolo[1,5-a][1,3,5]triazin-4-yl]-2,3,4,9-tetrahydro-1H-carbazol-3-amine (50 mg, crude) as a yellow solid. ES-LCMS m/z 468.2 [M+H]⁺.

### Step 2: (3R)—N-[2-(5-fluoro-3-pyridyl)-8-tetrahydrofuran-3-yl-pyrazolo[1,5-a][1,3,5]triazin-4-yl]-2,3,4,9-tetrahydro-1H-carbazol-3-amine (I-18)

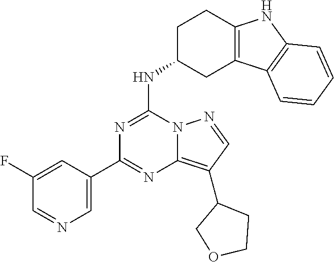 [SMILES: Fc1cncc(-c2nc(N[C@@H]3CCc4[nH]c5ccccc5c4C3)n3ncc(C4CCOC4)c3n2)c1]

A mixture of (3R)—N-[8-(2,5-dihydrofuran-3-yl)-2-(5-fluoro-3-pyridyl)pyrazolo[1,5-a][1,3,5]triazin-4-yl]-2,3,4,9-tetrahydro-1H-carbazol-3-amine (22.50 mg, 48.13 μmol, 1 eq) and Pd/C (0.05 mg, 10% purity) in THE (10 mL) was stirred at 25° C. for 0.5 hr under H2 (15 psi). The mixture was filtered and the filtrate was concentrated under reduced pressure to yield a residue which was purified by preparative HPLC (column: Agela ASB 150*25 mm*5 um; mobile phase: [water (0.05% HCl)−ACN]; B %: 60%-90%, 8 min) to yield (3R)—N-[2-(5-fluoro-3-pyridyl)-8-tetrahydrofuran-3-yl-pyrazolo[1,5-a][1,3,5]triazin-4-yl]-2,3,4,9-tetrahydro-1H-carbazol-3-amine (15.53 mg, 33.08 umol, 68.73% yield, 100% purity, [α]^{26.7}_{D}=+12.889 (MeOH, c=0.104 g/100 mL)) as a white solid. ¹H NMR (400 MHz, CD3OD) δ ppm 9.42 (br s, 1H), 8.62-8.53 (m, 2H), 8.04 (s, 1H), 7.38 (d, J=8.0 Hz, 1H), 7.28 (d, J=8.0 Hz, 1H), 7.07-7.00 (m, 1H), 6.98-6.94 (m, 1H), 4.83-4.80 (m, 1H), 4.21 (t, J=7.6 Hz, 1H), 4.12-4.10 (m, 1H), 4.03-3.95 (m, 1H), 3.89 (t, J=7.6 Hz, 1H), 3.75-3.74 (m, 1H), 3.29-3.27 (m, 1H), 3.11-2.88 (m, 3H), 2.50-2.36 (m, 2H), 2.32-2.19 (m, 2H); ES-LCMS m/z 470.3 [M+H]⁺.

### Example 20

### Synthesis of Compound I-19

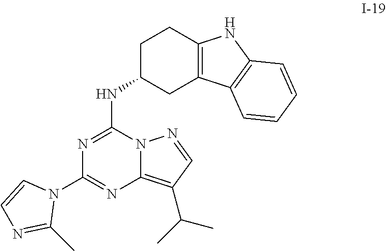 [SMILES: Cc1nccn1-c1nc(N[C@@H]2CCc3[nH]c4ccccc4c3C2)n2ncc(C(C)C)c2n1]

### Synthetic Scheme:

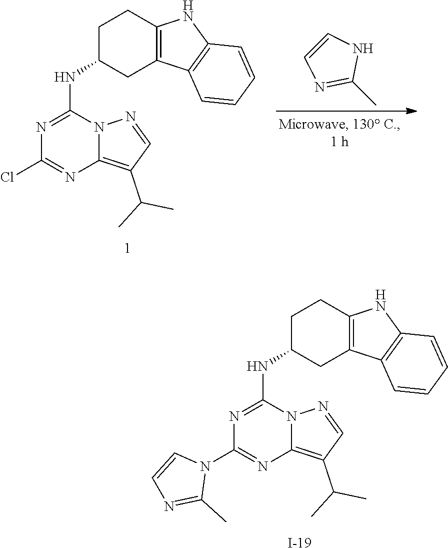

### Step 1: (3R)—N-[8-Isopropyl-2-(2-methylimidazol-1-yl)pyrazolo[1,5-a][1,3,5]triazin-4-yl]-2,3,4,9-tetrahydro-1H-carbazol-3-amine (I-19)

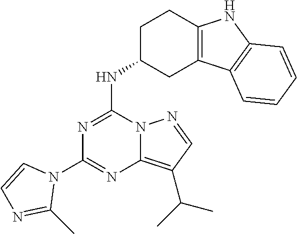 [SMILES: Cc1nccn1-c1nc(N[C@@H]2CCc3[nH]c4ccccc4c3C2)n2ncc(C(C)C)c2n1]

To a solution of (3R)—N-(2-chloro-8-isopropyl-pyrazolo[1,5-a][1,3,5]triazin-4-yl)-2,3,4,9-tetrahydro-1H-carbazol-3-amine (90 mg, 234.88 μmol, 1 eq) and 2-methyl-1H-imidazole (192.85 mg, 2.35 mmol, 10 eq) in DMF (3 mL) was added Cs₂CO₃ (229.59 mg, 704.64 μmol, 3 eq) under N₂ atmosphere. The mixture was degassed and purged with N₂ for three times and stirred at 130° C. for 1 h under microwave. The mixture was filtered, washed with MeOH (5 mL). The filtrate was concentrated to yield a residue which was purified by preparative HPLC (HCl condition; column: YMC-Actus Triart C18 100*30 mm*5 um; mobile phase: [water (0.05% HCl)−ACN]; B %: 20%-84%, 7 min). The desired fraction was lyophilized to yield (3R)—N-[8-isopropyl-2-(2-methylimidazol-1-yl)pyrazolo[1,5-a][1,3,5]triazin-4-yl]-2,3,4,9-tetrahydro-1H-carbazol-3-amine (54 mg, 116.64 μmol, 49.6% yield, 100.0% purity, HCl, [α]^{24.2}_{D}=+19.276, (MeOH, c=0.101 g/100 mL)) as a white solid. ¹H NMR (400 MHz, CD3OD) δ ppm 8.31 (d, J=2.2 Hz, 1H), 8.08 (s, 1H), 7.53 (d, J=2.2 Hz, 1H), 7.37 (d, J=7.6 Hz, 1H), 7.27 (d, J=8.1 Hz, 1H), 7.07-7.01 (m, 1H), 6.99-6.92 (m, 1H), 4.74 (m, 1H), 3.30-3.14 (m, 2H), 3.11 (s, 3H), 3.06-2.88 (m, 3H), 2.43-2.19 (m, 2H), 1.41 (d, J=7.1 Hz, 6H); ES-LCMS m/z 427.2 [M+H]⁺.

### Example 21

### Synthesis of Compound I-20a, I-20b and I-20c

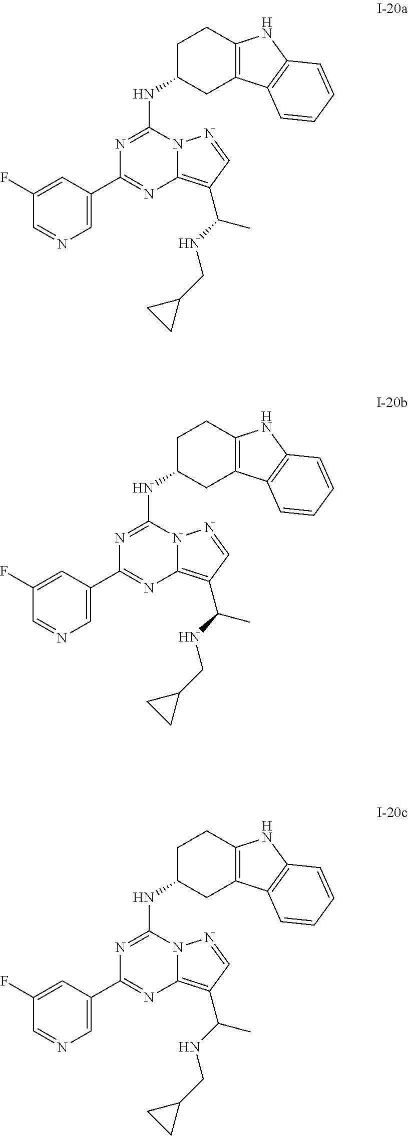 [SMILES: C[C@H](NCC1CC1)c1cnn2c(N[C@@H]3CCc4[nH]c5ccccc5c4C3)nc(-c3cncc(F)c3)nc12 ; C[C@@H](NCC1CC1)c1cnn2c(N[C@@H]3CCc4[nH]c5ccccc5c4C3)nc(-c3cncc(F)c3)nc12 ; CC(NCC1CC1)c1cnn2c(N[C@@H]3CCc4[nH]c5ccccc5c4C3)nc(-c3cncc(F)c3)nc12]

### Synthetic Scheme:

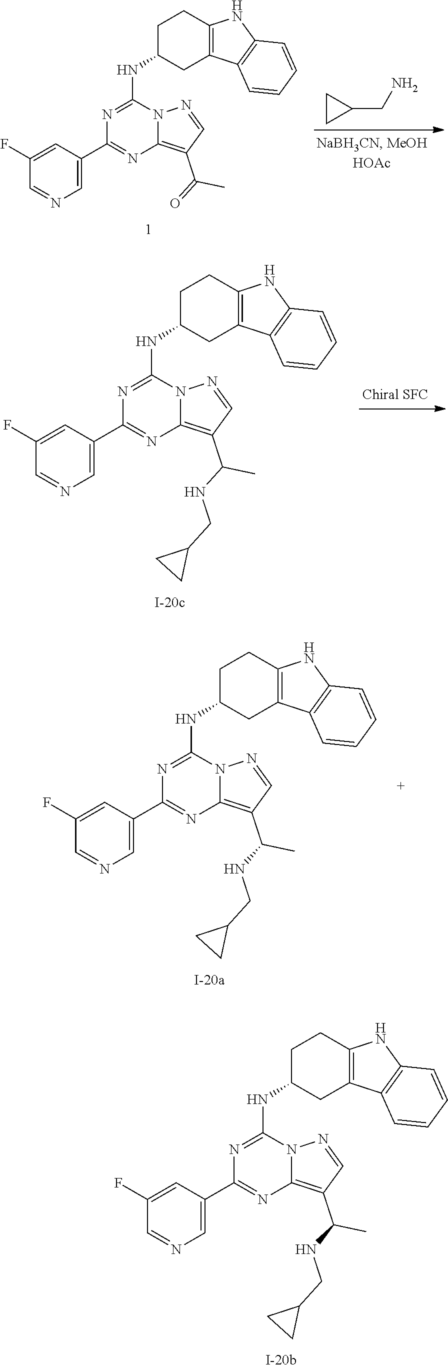

### Step 1: (3R)—N-[8-[(1S)-1-(Cyclopropylmethylamino)ethyl]-2-(5-fluoro-3-pyridyl)pyrazolo[1,5-a][1,3,5]triazin-4-yl]-2,3,4,9-tetrahydro-1H-carbazol-3-amine (I-20a) and (3R)—N-[8-[(1R)-1-(Cyclopropylmethylamino)ethyl]-2-(5-fluoro-3-pyridyl)pyrazolo[1,5-a][1,3,5]triazin-4-yl]-2,3,4,9-tetrahydro-1H-carbazol-3-amine (I-20b)

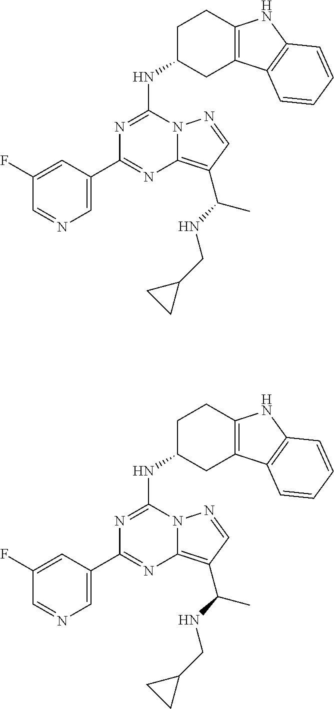 [SMILES: C[C@H](NCC1CC1)c1cnn2c(N[C@@H]3CCc4[nH]c5ccccc5c4C3)nc(-c3cncc(F)c3)nc12 ; C[C@@H](NCC1CC1)c1cnn2c(N[C@@H]3CCc4[nH]c5ccccc5c4C3)nc(-c3cncc(F)c3)nc12]

A mixture of 1-[2-(5-fluoro-3-pyridyl)-4-[[(3R)-2,3,4,9-tetrahydro-1H-carbazol-3-yl]amino] pyrazolo[1,5-a][1,3,5]triazin-8-yl]ethanone (200 μmg, 453.04 μmol, 1 eq), cyclopropylmethanamine (161.10 mg, 2.27 mmol, 5 eq) and Ti(i-PrO)₄ (386.28 mg, 1.36 mmol, 401.13 μL, 3 eq) in THF (20 mL) was degassed and purged with N₂ for 3 times then the mixture was stirred at 90° C. for 12 h under N₂ atmosphere. NaBH₄ (68.56 mg, 1.81 mmol, 4 eq) was added to the above mixture. The mixture was stirred at 25° C. for 2 h. The reaction mixture was filtrated through a pad of celite and the filtrate was concentrated to yield a residue which was purified by preparative TLC (EtOAc/MeOH=5/2, TLC: EtOAc/MeOH=5/1, R_{f}=0.40) to yield a residue which was separated by SFC (column: DAICEL CHIRALCEL OJ-H (250 mm*30 mm, 5 μm); mobile phase: [0.1% NH₃H₂O EtOH]; B %: 40%-40%). The solution after separation were concentrated to afford the crude products which were purified by preparative HPLC (HCl condition; column: YMC-Actus Triart C18 100*30 mm*5 um; mobile phase: [water (0.05% HCl)−ACN]; B %: 35%-65%, 10 min) twice, followed by lyophilization to yield an enantiomer (31.88 mg, 55.98 μmol, 12.3% yield, 100.0% purity, 2HCl, SFC: Rₜ=4.583 min, ee=99.4% and [α]^{25.5}_{D}=+11.005, MeOH, c=0.049 g/100 mL) as white solid. ¹H NMR (400 MHz, CD3OD) δ ppm 9.49 (s, 1H), 8.77-8.66 (m, 2H), 8.25 (s, 1H), 7.37 (d, J=7.8 Hz, 1H), 7.29 (d, J=8.3 Hz, 1H), 7.05 (t, J=7.6 Hz, 1H), 7.00-6.93 (m, 1H), 4.84 (m, 2H), 3.28 (m, 1H), 3.14-2.89 (m, 4H), 2.78 (dd, J=7.9, 13.1 Hz, 1H), 2.47-2.23 (m, 2H), 1.86 (d, J=7.1 Hz, 3H), 1.20-1.08 (m, 1H), 0.69 (d, J=6.8 Hz, 2H), 0.42-0.28 (m, 2H); ES-LCMS m/z 426.2 [M-C₄H₈NH]⁺; and the other enantiomer (29.55 mg, 51.89 μmol, 11.4% yield, 100.0% purity, 2HCl, SFC: Rₜ=4.980 min, ee=96.9% and [α]^{25.6}_{D}=+39.076, MeOH, c=0.050 g/100 mL) as white solid. ¹H NMR (400 MHz, CD3OD) δ ppm 9.49 (s, 1H), 8.73 (td, J=2.1, 9.5 Hz, 1H), 8.68 (d, J=2.7 Hz, 1H), 8.25 (s, 1H), 7.38 (d, J=7.8 Hz, 1H), 7.28 (d, J=8.1 Hz, 1H), 7.04 (t, J=7.1 Hz, 1H), 6.99-6.93 (m, 1H), 4.86-4.82 (m, 2H), 3.28 (m, 1H), 3.14-2.92 (m, 4H), 2.78 (dd, J=7.9, 12.8 Hz, 1H), 2.39 (s, 1H), 2.34-2.24 (m, 1H), 1.86 (d, J=7.1 Hz, 3H), 1.19-1.08 (m, 1H), 0.69 (d, J=8.1 Hz, 2H), 0.42-0.28 (m, 2H); ES-LCMS m z 426.2 [M-C₄H₈NH]⁺.

### Example 22

### Synthesis of Compound I-21

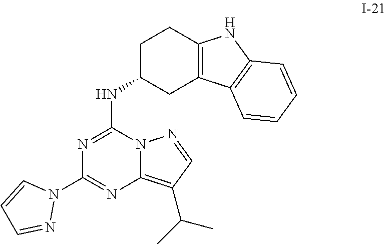 [SMILES: CC(C)c1cnn2c(N[C@@H]3CCc4[nH]c5ccccc5c4C3)nc(-n3cccn3)nc12]

### Synthetic Scheme:

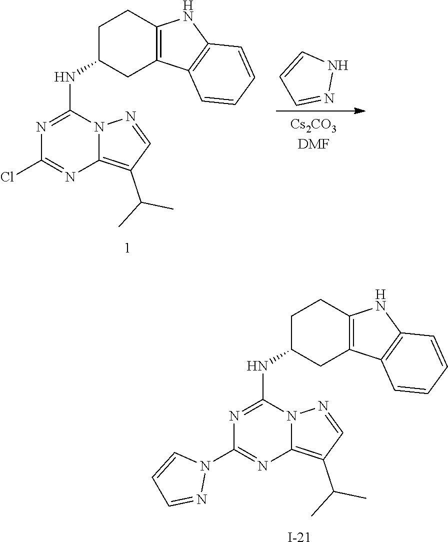

### Step 1: (3R)—N-(8-isopropyl-2-pyrazol-1-yl-pyrazolo[1,5-a][1,3,5]triazin-4-yl)-2,3,4,9-tetrahydro-1H-carbazol-3-amine (0-21)

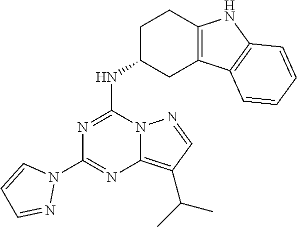 [SMILES: CC(C)c1cnn2c(N[C@@H]3CCc4[nH]c5ccccc5c4C3)nc(-n3cccn3)nc12]

To a solution of (3R)—N-(2-chloro-8-isopropyl-pyrazolo[1,5-a][1,3,5]triazin-4-yl)-2,3,4,9-tetrahydro-1H-carbazol-3-amine (70 mg, 182.69 μmol, 1 eq) in DMF (3 mL) was added 1H-pyrazole (124.37 mg, 1.83 mmol, 10 eq) and Cs₂CO₃ (178.57 mg, 548.06 μmol, 3 eq). The mixture was stirred at 130° C. for 1 h under microwave (1 bar). The reaction mixture was filtered and the filtrate was concentrated under reduced pressure to yield a residue which was purified by preparative HPLC (column: Agela ASB 150*25 mm*5 μm; mobile phase: [water (0.05% HCl)−ACN]; B %: 60%-90%, 8 min), followed by lyophilization to yield (3R)—N-(8-isopropyl-2-pyrazol-1-yl-pyrazolo[1,5-a][1,3,5]triazin-4-yl)-2,3,4,9-tetrahydro-1H-carbazol-3-amine (30 mg, 66.82 μmol, 36.6% yield, 100% purity, HCl, [α]^{25.6}_{D}=+15.272, MeOH, c=0.056 g/100 mL) as a white solid. ¹H NMR (400 MHz, DMSO-d₆) δ ppm 10.78 (s, 1H), 9.18 (d, J=8.6 Hz, 1H), 8.64 (d, J=2.7 Hz, 1H), 8.11 (s, 1H), 7.77 (s, 1H), 7.34 (d, J=7.6 Hz, 1H), 7.27 (d, J=8.1 Hz, 1H), 7.03-6.97 (m, 1H), 6.95-6.90 (m, 1H), 6.50 (dd, J=1.6, 2.6 Hz, 1H), 4.72 (s, 1H), 3.18-3.07 (m, 2H), 2.99-2.82 (m, 3H), 2.26-2.14 (m, 2H), 1.33 (s, 3H), 1.32 (s, 3H); ES-LCM S m/z 413.2 [M+H]⁺.

### Example 23

### Synthesis of Compound I-22

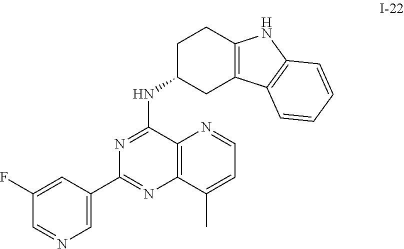 [SMILES: Cc1ccnc2c(N[C@@H]3CCc4[nH]c5ccccc5c4C3)nc(-c3cncc(F)c3)nc12]

### Synthetic Scheme:

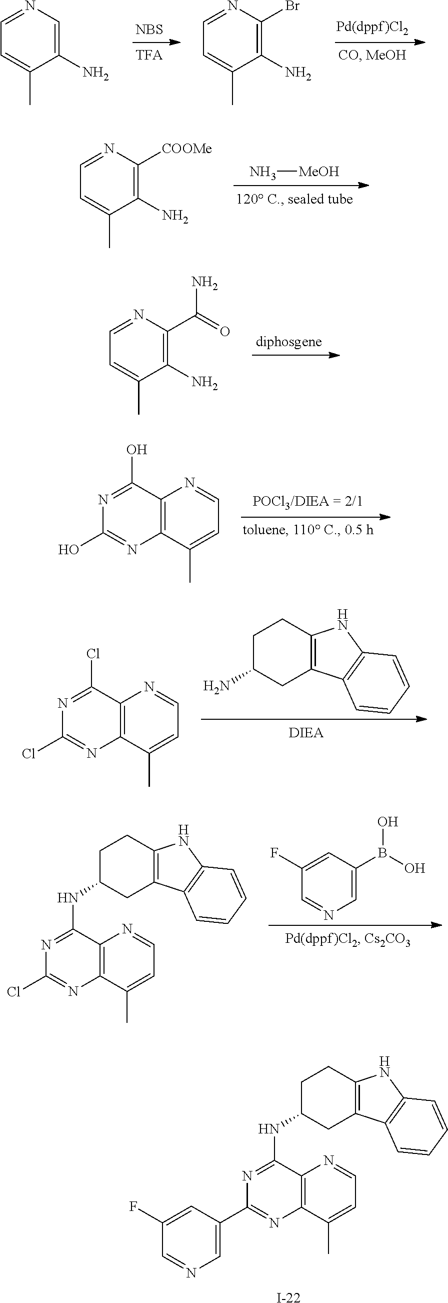

### Step 1: 2-Bromo-4-methyl-pyridin-3-amine

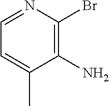

To a stirred solution of 4-methylpyridin-3-amine (3 g, 27.74 mmol, 1 eq) in TFA (40 mL) was added NBS (5.43 g, 30.52 mmol, 1.1 eq) slowly under ice-water bath. The reaction mixture was stirred at 25° C. for 12 h. The reaction mixture was diluted with water (150 mL), adjusted pH to 9 by 10% NaOH solution and extracted with EtOAc (100 mL×3). The combined organic layers were dried over Na₂SO₄ and concentrated to yield a residue which was purified by flash silica gel chromatography (from PE/EtOAc=100/1 to 1/1, TLC: PE/EtOAc=1/1, R_{f}=0.45) to yield 2-bromo-4-methyl-pyridin-3-amine (2.65 g, 14.05 mmol, 50.7% yield, 99.2% purity) as a brown solid. ¹H NMR (400 MHz, CD3OD) δ ppm 7.52 (d, J=4.6 Hz, 1H), 7.02 (d, J=4.6 Hz, 1H), 2.23 (s, 3H); ES-LCMS m/z 187.0, 189.0 [M+H]⁺.

### Step 2: Methyl 3-amino-4-methyl-pyridine-2-carboxylate

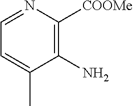

To a stirred solution of 2-bromo-4-methyl-pyridin-3-amine (2.67 g, 14.17 mmol, 1 eq) in MeOH (50 mL) was added Pd(dppf)Cl₂ (1.04 g, 1.42 mmol, 0.1 eq) and Et₃N (7.17 g, 70.84 mmol, 9.86 mL, 5 eq). The reaction mixture was degassed and purged with CO for three times and stirred at 60° C. for 48 h under CO atmosphere (50 psi). TLC (PE/EtOAc=10/1, R_{f}=0.30) showed starting material was consumed completely and one new spot was detected. The reaction mixture was concentrated to yield a residue which was purified by flash silica gel chromatography (from PE/EtOAc=100/1 to 1/1, TLC: PE/EtOAc=1/1, R_{f}=0.30) to yield methyl 3-amino-4-methyl-pyridine-2-carboxylate (2.25 g, 13.54 mmol, 95.6% yield, 100.0% purity) as a brown solid. ¹H NMR (400 MHz, CD3OD) δ ppm 7.75 (d, J=4.4 Hz, 1H), 7.20 (d, J=4.4 Hz, 1H), 3.91 (s, 3H), 2.22 (s, 3H).

### Step 3: 3-Amino-4-methyl-pyridine-2-carboxamide

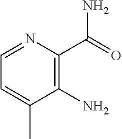

A mixture of methyl 3-amino-4-methyl-pyridine-2-carboxylate (500 mg, 3.01 mmol, 1 eq) and NH₃ (7 M in MeOH, 20 mL, 46.53 eq) was stirred at 125° C. for 12 h in a 100 mL sealed tube. The mixture was concentrated to yield 3-amino-4-methyl-pyridine-2-carboxamide (450 mg, 2.98 mmol, 98.9% yield, 100.0% purity) as a brown solid which was used in the next step without further purification. ¹H NMR (400 MHz, DMSO-d₆) δ ppm 7.99 (s, 1H), 7.70 (d, J=4.2 Hz, 1H), 7.31 (s, 1H), 7.14 (d, J=4.2 Hz, 1H), 6.76 (s, 2H), 2.13 (s, 3H); ES-LCMS m/z 152.1 [M+H]⁺.

### Step 4: 8-Methylpyrido[3,2-d]pyrimidine-2,4-diol

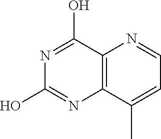

To a stirred solution of 3-amino-4-methyl-pyridine-2-carboxamide (400 mg, 2.65 mmol, 1 eq) in 1,4-dioxane (6 mL) and THE (3 mL) was added trichloromethyl carbonochloridate (628.18 mg, 3.18 mmol, 383.04 μL, 1.2 eq). The reaction mixture was stirred at 80° C. for 2 h. The mixture was washed with EtOH (30 mL×3). The solid was filtered and concentrated to yield 8-methylpyrido[3,2-d]pyrimidine-2,4-diol (450 mg, 2.54 mmol, 96.0% yield, 100.0% purity) as a gray solid which was used in the next step without further purification. ¹H NMR (400 MHz, DMSO-d₆) δ ppm 11.77 (s, 1H), 10.90 (s, 1H), 8.39 (d, J=4.6 Hz, 1H), 7.65 (d, J=4.4 Hz, 1H), 2.44 (s, 3H); ES-LCMS m/z 178.1 [M+H]⁺.

### Step 5: 2,4-Dichloro-8-methyl-pyrido[3,2-d]pyrimidine

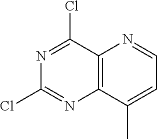

To a stirred solution of 8-methylpyrido[3,2-d]pyrimidine-2,4-diol (200 mg, 1.13 mmol, 1 eq) in toluene (5 mL) was added POCl₃ (3.70 g, 24.13 mmol, 2.24 mL, 21.37 eq) and DIEA (742.00 mg, 5.74 mmol, 1.00 mL, 5.09 eq). The reaction mixture was stirred at 110° C. for 30 min. The reaction mixture was concentrated to yield 2,4-dichloro-8-methyl-pyrido[3,2-d]pyrimidine (240 mg, crude) as brown oil which was used in the next step without further purification. ES-LCMS m/z 214.1, 216.1 [M+H]⁺.

### Step 6: (3R)—N-(2-Chloro-8-methyl-pyrido[3,2-d]pyrimidin-4-yl)-2,3,4,9-tetrahydro-1H-carbazol-3-amine

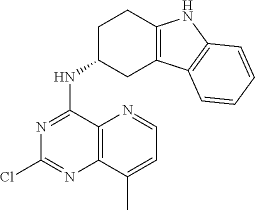 [SMILES: Cc1ccnc2c(N[C@@H]3CCc4[nH]c5ccccc5c4C3)nc(Cl)nc12]

To a stirred solution of 2,4-dichloro-8-methyl-pyrido[3,2-d]pyrimidine (240 mg, 1.12 mmol, 1 eq) in MeCN (10 mL) was added DIEA (2.70 g, 20.88 mmol, 3.64 mL, 18.62 eq) and (3R)-2,3,4,9-tetrahydro-1H-carbazol-3-amine (120 mg, 644.29 μmol, 0.58 eq). The reaction mixture was stirred at 60° C. for 3 h. The reaction mixture was concentrated to yield a residue which was purified by flash silica gel chromatography (from PE/EtOAc=100/1 to 2/1, TLC: PE/EtOAc=3/1, R_{f}=0.35) to yield (3R)—N-(2-chloro-8-methyl-pyrido[3,2-d]pyrimidin-4-yl)-2,3,4,9-tetrahydro-1H-carbazol-3-amine (150 mg, 318.68 μmol, 28.4% yield, 77.3% purity) as yellow oil. ¹H NMR (400 MHz, CD3OD) δ ppm 8.52 (d, J=4.4 Hz, 1H), 7.57 (dd, J=0.9, 4.5 Hz, 1H), 7.37 (d, J=7.8 Hz, 1H), 7.26 (d, J=8.1 Hz, 1H), 7.06-7.00 (m, 1H), 6.99-6.92 (m, 1H), 4.75-4.67 (m, 1H), 3.21 (dd, J=5.1, 14.7 Hz, 1H), 3.02-2.81 (m, 3H), 2.59 (s, 3H), 2.32-2.14 (m, 2H); ES-LCMS m/z 364.1, 366.1 [M+H]⁺.

### Step 7: 2,4-Dichloro-8-methyl-pyrido[3,2-d]pyrimidine (I-22)

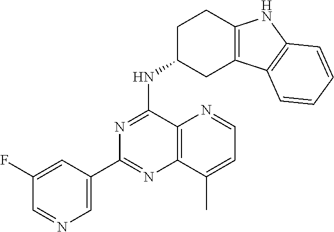 [SMILES: Cc1ccnc2c(N[C@@H]3CCc4[nH]c5ccccc5c4C3)nc(-c3cncc(F)c3)nc12]

To a solution of (3R)—N-(2-chloro-8-methyl-pyrido[3,2-d]pyrimidin-4-yl)-2,3,4,9-tetrahydro-1H-carbazol-3-amine (75 mg, 159.34 μmol, 1 eq) in 1,4-dioxane (3 mL) and water (1 mL) was added Cs₂CO₃ (155.75 mg, 478.02 μmol, 3 eq), Pd(dppf)Cl₂ (11.66 mg, 15.93 μmol, 0.1 eq) and (5-fluoro-3-pyridyl)boronic acid (56.13 mg, 398.35 μmol, 2.5 eq). The mixture was bubbled with N₂ for 3 min and stirred at 110° C. for 0.5 h under microwave. The reaction mixture was filtered through a pad of celite and concentrated to yield a residue which was purified by preparative HPLC (column: Xtimate C18 150*25 mm*5 μm; mobile phase: [water (0.05% HCl)−ACN]; B %: 65%-95%, 8 min). The desired fraction was lyophilized to yield (3R)—N-[2-(5-fluoro-3-pyridyl)-8-methyl-pyrido[3,2-d]pyrimidin-4-yl]-2,3,4,9-tetrahydro-1H-carbazol-3-amine (47.77 mg, 96.04 μmol, 60.3% yield, 100.0% purity, 2HCl, [α]^{25.2}_{D}=+15.251, MeOH, c=0.113 g/100 mL) as a yellow solid. ¹H NMR (400 MHz, CD3OD) δ ppm 9.45 (s, 1H), 8.79-8.72 (m, 2H), 8.66 (d, J=4.4 Hz, 1H), 7.71 (d, J=3.9 Hz, 1H), 7.37 (d, J=7.8 Hz, 1H), 7.28 (d, J=7.8 Hz, 1H), 7.04 (t, J=7.1 Hz, 1H), 6.99-6.92 (m, 1H), 4.98 (d, J=9.3 Hz, 1H), 3.28 (s, 1H), 3.10-2.90 (m, 3H), 2.78 (s, 3H), 2.44-2.23 (m, 2H); ES-LCMS m/z 425.3 [M+H]⁺.

### Example 24

### Synthesis of Compound I-23

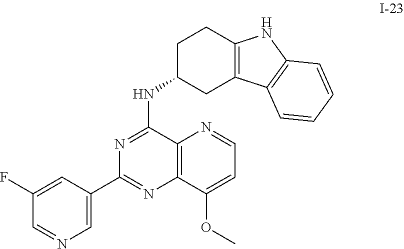 [SMILES: COc1ccnc2c(N[C@@H]3CCc4[nH]c5ccccc5c4C3)nc(-c3cncc(F)c3)nc12]

### Synthetic Scheme:

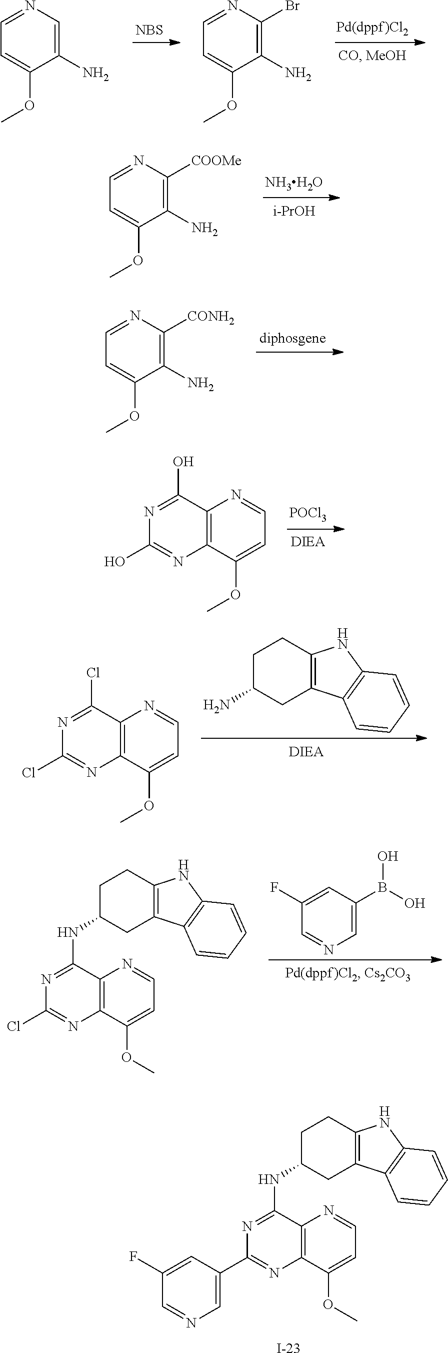

### Step 1: 2-Bromo-4-methoxy-pyridin-3-amine

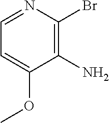

To a solution of 4-methoxypyridin-3-amine (9 g, 72.50 mmol, 1 eq) in TFA (100 mL) was stirred at ice bath temperature and was added NBS (14.19 g, 79.75 mmol, 1.1 eq) in several batches. The mixture was stirred at 20° C. for 12 h. The reaction mixture was concentrated under reduced pressure to yield a residue which was diluted with ice, then aq NaHCO₃ was added to above solution until pH=8, extracted with EtOAc (50 mL×3). The combined organic layers were dried over Na₂SO₄, filtered and concentrated under reduced pressure to yield a residue which was purified by flash silica gel chromatography (from PE/EtOAC=100/1 to 1/1, TLC: PE/EtOAc=1/1, R_{f}=0.4) to yield 2-bromo-4-methoxy-pyridin-3-amine (12 g, 59.10 mmol, 81.5% yield, 100.0% purity) as a white solid. ¹H NMR (400 MHz, CDCl₃) δ ppm 7.73 (d, J=5.1 Hz, 1H), 6.67 (d, J=5.1 Hz, 1H), 4.09 (d, J=9.5 Hz, 2H), 3.90 (s, 3H); ES-LCMS m/z 203.0, 205.0 [M+H]⁺.

### Step 2: Methyl 3-amino-4-methoxy-pyridine-2-carboxylate

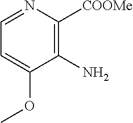

To a solution of 2-bromo-4-methoxy-pyridin-3-amine (6 g, 29.55 mmol, 1 eq) in MeOH (15 mL) was added Pd(dppf)Cl₂ (2.16 g, 2.96 mmol, 0.1 eq) and Et₃N (14.95 g, 147.76 mmol, 20.57 mL, 5 eq) under N₂ atmosphere. The mixture was degassed and purged with CO for 3 times. The mixture was stirred under CO (50 psi) at 60° C. for 48 h. The reaction mixture was concentrated under reduced pressure to yield a residue which was purified by flash silica gel chromatography (from PE/EtOAC=100/1 to 1/1, TLC: PE/EtOAc=1/1, R_{f}=0.2) to yield methyl 3-amino-4-methoxy-pyridine-2-carboxylate (4.5 g, 24.48 mmol, 82.8% yield, 99.1% purity) as a brown solid. ¹H NMR (400 MHz, CD3OD) δ ppm 7.80 (d, J=5.1 Hz, 1H), 6.96 (d, J=5.1 Hz, 1H), 3.97 (s, 3H), 3.93-3.89 (m, 3H); ES-LCMS m/z 183.1 [M+H]⁺.

### Step 3: 3-Amino-4-methoxy-pyridine-2-carboxamide

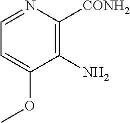

A solution of methyl 3-amino-4-methoxy-pyridine-2-carboxylate (1 g, 5.49 mmol, 1 eq) in NH₃/MeOH (7 M, 30 mL) was stirred in a sealed tube at 120° C. for 12 h. The reaction mixture was filtered and concentrated under reduced pressure to yield 3-amino-4-methoxy-pyridine-2-carboxamide (800 mg, 4.76 mmol, 86.8% yield, 99.5% purity) as a brown solid which was used in the next step without further purification. ¹H NMR (400 MHz, DMSO-d₆) δ ppm 7.92 (s, 1H), 7.74 (d, J=5.1 Hz, 1H), 7.32 (s, 1H), 6.95 (d, J=4.9 Hz, 1H), 6.49 (s, 2H), 3.87 (s, 3H); ES-LCMS m/z 168.1 [M+H]⁺.

### Step 4: N′-(4-Isopropyl-1H-pyrazol-5-yl)pyridazine-4-carboxamidine

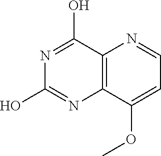

To a solution of 3-amino-4-methoxy-pyridine-2-carboxamide (500 mg, 2.98 mmol, 1 eq) in 1,4-dioxane (10 mL) and THE (5 mL) was added diphosgene (647.65 mg, 3.27 mmol, 394.91 μL, 1.1 eq). The mixture was stirred at 40° C. for 6 h. The reaction mixture was filtered and concentrated under reduced pressure to yield 8-methoxypyrido[3,2-d]pyrimidine-2,4-diol (200 mg, 783.90 μmol, 26.3% yield, 90.0% purity, HCl) as a gray solid which was used in the next step without further purification. ¹H NMR (400 MHz, DMSO-d₆) δ ppm 12.02 (s, 1H), 11.46 (s, 1H), 8.54 (d, J=6.1 Hz, 1H), 7.57 (d, J=6.1 Hz, 1H), 4.10 (s, 3H); ES-LCMS m/z 194.1 [M+H]⁺.

### Step 5: 2,4-Dichloro-8-methoxy-pyrido[3,2-d]pyrimidine

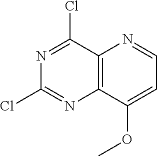

To a solution of 8-methoxypyrido[3,2-d]pyrimidine-2,4-diol (160 mg, 745.50 μmol, 1 eq) in toluene (3 mL) was added POCl₃ (1.65 g, 10.76 mmol, 1 mL, 14.43 eq) and DIEA (289.05 mg, 2.24 mmol, 389.56 μL, 3 eq). The mixture was stirred at 110° C. for 1 h. The reaction mixture was concentrated under reduced pressure to yield 2,4-dichloro-8-methoxy-pyrido[3,2-d]pyrimidine (171.5 mg, crude) as a brown solid which was used in the next step without further purification. ES-LCMS m/z 230.0, 232.1 [M+H]⁺.

### Step 6: (3R)—N-(2-Chloro-8-methoxy-pyrido[3,2-d]pyrimidin-4-yl)-2,3,4,9-tetrahydro-1H-carbazol-3-amine

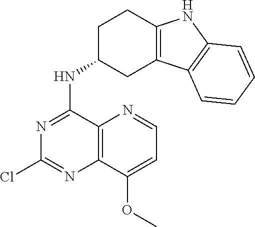 [SMILES: COc1ccnc2c(N[C@@H]3CCc4[nH]c5ccccc5c4C3)nc(Cl)nc12]

A mixture of 2,4-dichloro-8-methoxy-pyrido[3,2-d]pyrimidine (171.5 mg, 745.49 μmol, 1 eq), (3R)-2,3,4,9-tetrahydro-1H-carbazol-3-amine (145.79 mg, 782.76 μmol, 1.05 eq) and DIEA (289.05 mg, 2.24 mmol, 389.55 μL, 3 eq) in CH₃CN (4 mL) was degassed and purged with N₂ for 3 times, the mixture was stirred at 60° C. for 3 h under N₂ atmosphere. The reaction mixture was concentrated under reduced pressure to yield a residue which was purified by flash silica gel chromatography (from PE/EtOAC=100/1 to 1/1, TLC: PE/EtOAc=1/1, R_{f}=0.3) to yield (3R)—N-(2-chloro-8-methoxy-pyrido[3,2-d]pyrimidin-4-yl)-2,3,4,9-tetrahydro-1H-carbazol-3-amine (120 mg, 291.91 μmol, 39.2% yield, 92.4% purity) as a yellow solid. ¹H NMR (400 MHz, CDCl₃) δ ppm 8.46 (d, J=5.4 Hz, 1H), 7.85 (s, 1H), 7.52-7.42 (m, 2H), 7.34 (d, J=8.1 Hz, 1H), 7.22-7.09 (m, 2H), 6.99 (d, J=5.4 Hz, 1H), 5.02-4.74 (m, 1H), 4.06 (s, 3H), 3.30 (dd, J=5.0, 15.5 Hz, 1H), 3.02-2.85 (m, 3H), 2.33-2.23 (m, 2H) ES-LCMS m/z 380.2, 381.2[M+H]⁺.

### Step 7: (3R)—N-[2-(5-fluoro-3-pyridyl)-8-methoxy-pyrido[3,2-d]pyrimidin-4-yl]-2,3,4,9-tetrahydro-1H-carbazol-3-amine (I-23)

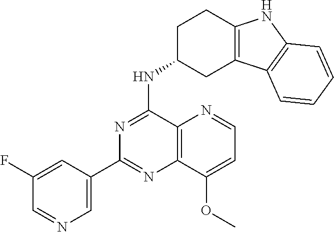 [SMILES: COc1ccnc2c(N[C@@H]3CCc4[nH]c5ccccc5c4C3)nc(-c3cncc(F)c3)nc12]

(3R)—N-(2-chloro-8-methoxy-pyrido[3,2-d]pyrimidin-4-yl)-2,3,4,9-tetrahydro-1H-carbazol-3-amine (50 mg, 121.10 μmol, 1 eq), (5-fluoro-3-pyridyl)boronic acid (20.48 mg, 145.32 μmol, 1.2 eq), Cs₂CO₃ (118.37 mg, 363.31 μmol, 3 eq) and Pd(dppf)Cl₂ (8.86 mg, 12.11 μmol, 0.1 eq) were taken up into a microwave tube in 1,4-dioxane (2 mL) and H₂O (0.5 mL). The sealed tube was heated at 110° C. for 0.5 h under microwave. The reaction mixture was filtered and concentrated under reduced pressure to yield a residue which was purified by preparative HPLC (column: Agela ASB 150*25 mm*5 um; mobile phase: [water (0.05% HCl)−ACN]; B %: 55%-85%, 8 min) followed by lyophilization to yield (3R)—N-[2-(5-fluoro-3-pyridyl)-8-methoxy-pyrido[3,2-d]pyrimidin-4-yl]-2,3,4,9-tetrahydro-1H-carbazol-3-amine (25.66 mg, 49.98 μmol, 41.3% yield, 2HCl, [α]^{25.2}_{D}=+23.669, MeOH, c=0.113 g/100 mL) as a yellow solid. ¹H NMR (400 MHz, CD3OD) δ ppm 9.31 (s, 1H), 8.72 (d, J=5.4 Hz, 1H), 8.70 (d, J=2.4 Hz, 1H), 8.55-8.50 (m, 1H), 7.45 (d, J=5.4 Hz, 1H), 7.37 (d, J=7.8 Hz, 1H), 7.28 (d, J=8.1 Hz, 1H), 7.07-7.01 (m, 1H), 6.99-6.89 (m, 1H), 5.03-4.96 (m, 1H), 4.19 (s, 3H), 3.28 (d, J=5.4 Hz, 1H), 3.08-2.91 (m, 3H), 2.43-2.25 (m, 2H); ES-LCMS m/z 441.2 [M+H]⁺.

### Example 25

### Synthesis of Compound I-24a and I-24b

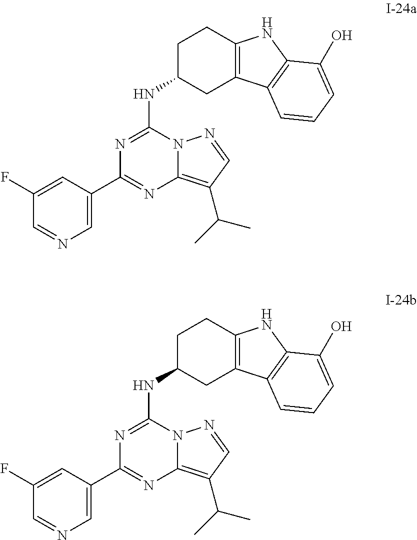 [SMILES: CC(C)c1cnn2c(N[C@@H]3CCc4[nH]c5c(O)cccc5c4C3)nc(-c3cncc(F)c3)nc12 ; CC(C)c1cnn2c(N[C@H]3CCc4[nH]c5c(O)cccc5c4C3)nc(-c3cncc(F)c3)nc12]

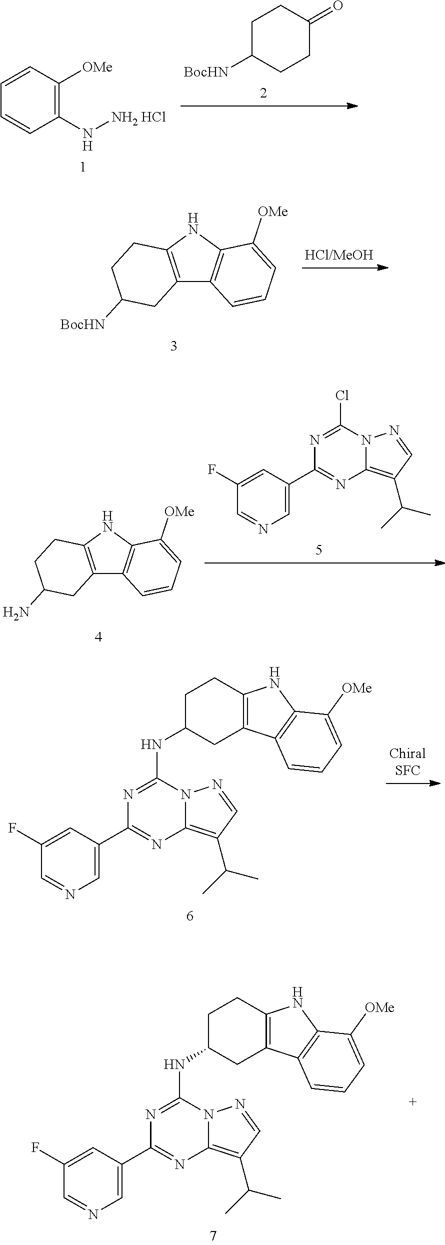

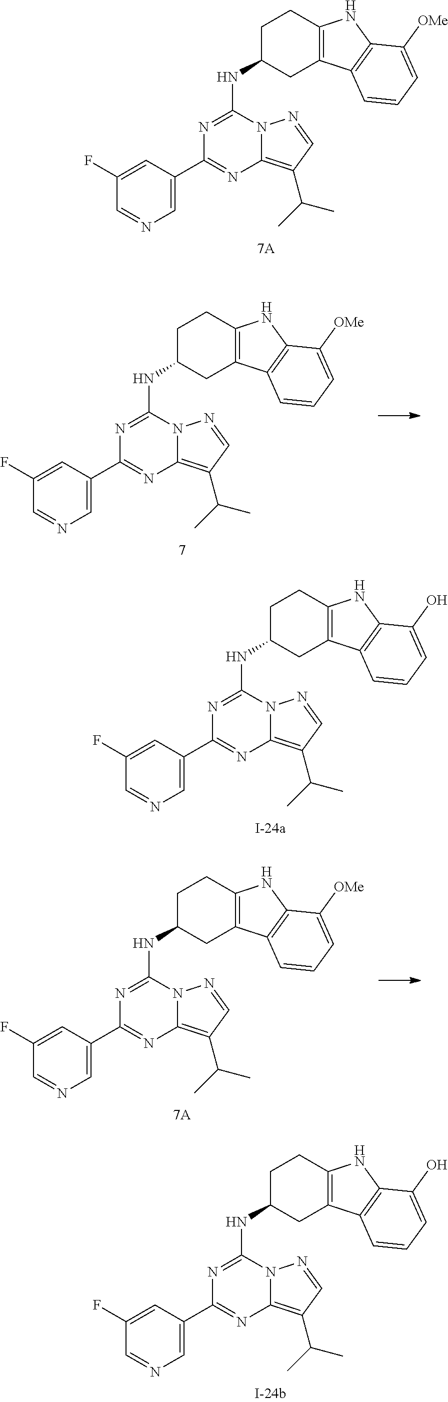 [SMILES: COc1cccc2c3c([nH]c12)CC[C@H](Nc1nc(-c2cncc(F)c2)nc2c(C(C)C)cnn12)C3 ; COc1cccc2c3c([nH]c12)CC[C@@H](Nc1nc(-c2cncc(F)c2)nc2c(C(C)C)cnn12)C3 ; CC(C)c1cnn2c(N[C@@H]3CCc4[nH]c5c(O)cccc5c4C3)nc(-c3cncc(F)c3)nc12 ; COc1cccc2c3c([nH]c12)CC[C@H](Nc1nc(-c2cncc(F)c2)nc2c(C(C)C)cnn12)C3 ; CC(C)c1cnn2c(N[C@H]3CCc4[nH]c5c(O)cccc5c4C3)nc(-c3cncc(F)c3)nc12]

### Step 1: tert-Butyl N-(8-methoxy-2,3,4,9-tetrahydro-1H-carbazol-3-yl)carbamate

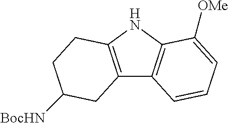 [SMILES: COc1cccc2c3c([nH]c12)CCC(NC(=O)OC(C)(C)C)C3]

To a solution of (2-methoxyphenyl)hydrazine (1.64 g, 9.38 mmol, 1 eq, HCl) in EtOH (30 mL) was added tert-butyl N-(4-oxocyclohexyl)carbamate (2 g, 9.38 mmol, 2.00 mL, 1 eq). The mixture was stirred at 70° C. for 1 h under N₂ atmosphere. The mixture was concentrated and water (20 mL) was added, extracted with EtOAc (50 mL×3). The combined organic layers were washed with brine (20 mL), dried over Na₂SO₄, filtered and concentrated. The crude material was purified on silica gel column chromatography (from pure PE to PE/EtOAc=2/1, TLC: PE/EtOAc=3/1, R_{f}=0.5) to yield tert-butyl N-(8-methoxy-2,3,4,9-tetrahydro-1H-carbazol-3-yl)carbamate (0.6 g, 1.71 mmol, 18.2% yield, 90.0% purity) as a yellow solid. ¹H NMR (400 MHz, CDCl₃) δ ppm 7.98 (s, 1H), 7.08-6.98 (m, 2H), 6.62 (d, J=7.3 Hz, 1H), 4.72 (s, 1H), 4.12 (s, 1H), 3.94 (s, 3H), 3.07 (dd, J=5.1, 15.4 Hz, 1H), 2.83 (d, J=5.6 Hz, 2H), 2.59 (dd, J=5.7, 14.8 Hz, 1H), 2.17-2.06 (m, 1H), 1.98 (d, J=6.1 Hz, 1H), 1.45 (s, 9H); ES-LCMS m/z 317.2 [M+H]⁺.

### Step 2: 8-Methoxy-2,3,4,9-tetrahydro-1H-carbazol-3-amine

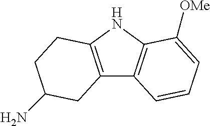 [SMILES: COc1cccc2c3c([nH]c12)CCC(N)C3]

A solution of tert-butyl N-(8-methoxy-2,3,4,9-tetrahydro-1H-carbazol-3-yl)carbamate (300 mg, 853.37 μmol, 1 eq) in HCl (4 M in MeOH, 5 mL) was stirred at 20° C. for 1 h. The mixture was concentrated to yield 8-methoxy-2,3,4,9-tetrahydro-1H-carbazol-3-amine (250 mg, 778.01 mol, 91.2% yield, 90.0% purity, 2 HCl) as a brown solid. ¹H NMR (400 MHz, CD3OD) δ ppm 7.01-6.96 (m, 1H), 6.94-6.88 (m, 1H), 6.62 (d, J=7.6 Hz, 1H), 3.93 (s, 3H), 3.65 (s, 1H), 3.17 (dd, J=5.0, 14.8 Hz, 1H), 2.94 (t, J=6.1 Hz, 2H), 2.75 (dd, J=8.6, 14.9 Hz, 1H), 2.27 (dd, J=3.4, 8.3 Hz, 1H), 2.11-1.99 (m, 1H); ES-LCMS m/z 217.1 [M+H]⁺.

### Step 3: (3R)—N-[2-(5-fluoro-3-pyridyl)-8-isopropyl-pyrazolo[1,5-a][1,3,5]triazin-4-yl]-8-methoxy-2,3,4,9-tetrahydro-1H-carbazol-3-amine and (3S)—N-[2-(5-fluoro-3-pyridyl)-8-isopropyl-pyrazolo[1,5-a][1,3,5]triazin-4-yl]-8-methoxy-2,3,4,9-tetrahydro-1H-carbazol-3-amine

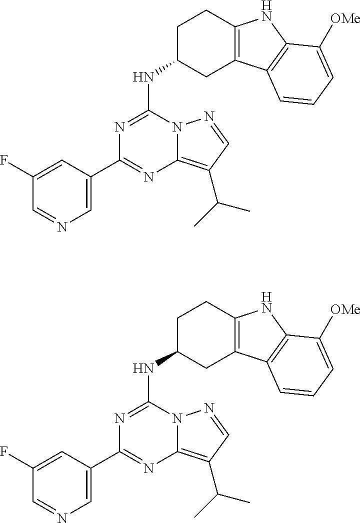 [SMILES: COc1cccc2c3c([nH]c12)CC[C@@H](Nc1nc(-c2cncc(F)c2)nc2c(C(C)C)cnn12)C3 ; COc1cccc2c3c([nH]c12)CC[C@H](Nc1nc(-c2cncc(F)c2)nc2c(C(C)C)cnn12)C3]

To a solution of 4-chloro-2-(5-fluoro-3-pyridyl)-8-isopropyl-pyrazolo[1,5-a][1,3,5]triazine (200 mg, 667.78 μmol, 1 eq) in ACN (8 mL) was added DIEA (401.50 mg, 3.11 mmol, 541.11 μL, 4.65 eq) and 8-methoxy-2,3,4,9-tetrahydro-1H-carbazol-3-amine (214.58 mg, 667.78 μmol, 1 eq, 2 HCl). The mixture was stirred at 50° C. for 2 h. The mixture was concentrated and water (20 mL) was added, extracted with EtOAc (40 mL×3). The combined organic layers were washed with brine (15 mL), dried over Na₂SO₄, filtered and concentrated. The residue was further separated by SFC (column: DAICEL CHIRALCEL OD (250 mm*30 mm, 10 μm); mobile phase: [0.1% NH₃.H₂OMeOH]; B %: 35%-35%) to yield Peak 1 and Peak 2. One of these peaks was concentrated to yield an intermediate enantiomer (110 mg, 233.28 μmol, 34.9% yield, 100.0% purity. SFC: Rₜ=5.628 min, ee=100%) as a white solid. ¹H NMR (400 MHz, CDCl₃) δ ppm 9.51 (s, 1H), 8.55 (d, J=2.7 Hz, 1H), 8.44 (d, J=9.5 Hz, 1H), 8.10 (s, 1H), 7.84 (s, 1H), 7.11-6.98 (m, 2H), 6.75-6.61 (m, 2H), 4.93 (s, 1H), 3.97 (s, 3H), 3.39-3.21 (m, 2H), 3.05-2.89 (m, 3H), 2.42-2.25 (m, 2H), 1.40 (d, J=7.1 Hz, 6H); ES-LCMS m/z 472.2 [M+H]⁺. The other of these peaks was concentrated to yield the other intermediate enantiomer (100 mg, 208.23 μmol, 31.2% yield, 98.2% purity. SFC: Rₜ=4.728 min, ee=98.698%) as a white solid. ¹H NMR (400 MHz, CDCl₃) δ ppm 9.51 (s, 1H), 8.55 (d, J=2.4 Hz, 1H), 8.44 (d, J=9.5 Hz, 1H), 8.09 (s, 1H), 7.84 (s, 1H), 7.11-6.97 (m, 2H), 6.74-6.58 (m, 2H), 4.93 (s, 1H), 3.97 (s, 3H), 3.38-3.20 (m, 2H), 3.04-2.87 (m, 3H), 2.42-2.27 (m, 2H), 1.40 (d, J=7.1 Hz, 6H); ES-LCMS m/z 472.2 [M+H]⁺.

### Step 4: (6R)-6-[[2-(5-fluoro-3-pyridyl)-8-isopropyl-pyrazolo[1,5-a][1,3,5]triazin-4-yl]amino]-6,7,8,9-tetrahydro-5H-carbazol-1-ol (I-24a)

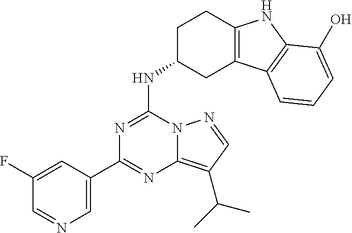 [SMILES: CC(C)c1cnn2c(N[C@@H]3CCc4[nH]c5c(O)cccc5c4C3)nc(-c3cncc(F)c3)nc12]

To a solution of one of the intermediate enantiomers (105.00 mg, 222.68 μmol, 1 eq) in DCM (12 mL) was added AlCl₃ (890.77 mg, 6.68 mmol, 365.07 μL, 30 eq) under N₂ atmosphere. The mixture was stirred at 40° C. for 12 h under N₂ atmosphere. To the mixture was added water (40 mL), extracted with EtOAc (60 mL×2). The combined organic layers were washed with brine (50 mL), dried over Na₂SO₄, filtered and concentrated. The residue was purified by preparative HPLC (column: Agela ASB 150*25 mm*5 μm; mobile phase: [water (0.05% HCl)−ACN]; B %: 70%-100%, 8 min) and lyophilized to yield an enantiomer (22.22 mg, 41.15 μmol, 18.5% yield, 98.2% purity, 2 HCl, SFC: Rₜ=3.727 min, ee=99.69%. [α]^{24.7}_{D}=+8.932, MeOH, c=0.054 g/100 mL) as a white solid. ¹H NMR (400 MHz, DMSO-d₆) δ ppm 10.56 (s, 1H), 9.37 (s, 1H), 9.00 (d, J=8.5 Hz, 1H), 8.70 (d, J=2.8 Hz, 1H), 8.49-8.35 (m, 1H), 8.14 (s, 1H), 6.90-6.75 (m, 1H), 6.72 (t, J=7.7 Hz, 1H), 6.45 (d, J=7.3 Hz, 1H), 4.79 (s, 1H), 3.23 (td, J=7.0, 13.9 Hz, 1H), 3.11-2.81 (m, 4H), 2.18 (d, J=4.5 Hz, 2H), 1.37 (d, J=7.0 Hz, 6H); ES-LCMS m/z 458.3 [M+H]⁺.

### Step 5: (6S)-6-[[2-(5-fluoro-3-pyridyl)-8-isopropyl-pyrazolo[1,5-a][1,3,5]triazin-4-yl]amino]-6,7,8,9-tetrahydro-5H-carbazol-1-ol (I-24b)

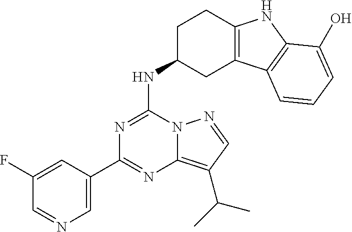 [SMILES: CC(C)c1cnn2c(N[C@H]3CCc4[nH]c5c(O)cccc5c4C3)nc(-c3cncc(F)c3)nc12]

To a solution of the other intermediate enantiomer (95 mg, 197.82 μmol, 1 eq) in DCM (12 mL) was added AlCl₃ (791.30 mg, 5.93 mmol, 324.30 μL, 30 eq) under N₂ atmosphere. The mixture was stirred at 40° C. for 12 h under N₂ atmosphere. To the mixture was added water (40 mL), extracted with EtOAc (60 mL×2). The combined organic layers were washed with brine (50 mL), dried over Na₂SO₄, filtered and concentrated. The residue was purified by preparative HPLC (column: Agela ASB 150*25 mm*5 μm; mobile phase: [water (0.05% HCl)−ACN]; B %: 70%-100%, 8 min) and lyophilized to yield the other enantiomer (21.35 mg, 40.25 μmol, 20.4% yield, 100.0% purity, 2HCl, SFC: Rₜ=4.584 min, ee=98.126%. [α]^{24.7}_{D}=−56.292, MeOH, c=0.056 g/100 mL) as a white solid. ¹H NMR (400 MHz, DMSO-d₆) δ ppm 10.57 (s, 1H), 9.37 (s, 1H), 9.00 (d, J=8.8 Hz, 1H), 8.70 (d, J=2.8 Hz, 1H), 8.49-8.35 (m, 1H), 8.15 (s, 1H), 6.84-6.78 (m, 1H), 6.75-6.69 (m, 1H), 6.45 (d, J=7.0 Hz, 1H), 4.89-4.73 (m, 1H), 3.23 (td, J=6.9, 14.0 Hz, 1H), 3.11-2.82 (m, 4H), 2.18 (d, J=3.8 Hz, 2H), 1.37 (d, J=6.8 Hz, 6H); ES-LCMS m/z 458.2 [M+H]⁺.

### Example 26

### Synthesis of Compound I-25a and I-25b

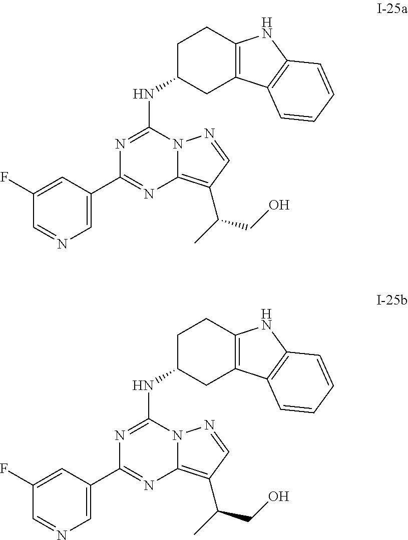 [SMILES: C[C@@H](CO)c1cnn2c(N[C@@H]3CCc4[nH]c5ccccc5c4C3)nc(-c3cncc(F)c3)nc12 ; C[C@H](CO)c1cnn2c(N[C@@H]3CCc4[nH]c5ccccc5c4C3)nc(-c3cncc(F)c3)nc12]

### Synthetic Scheme:

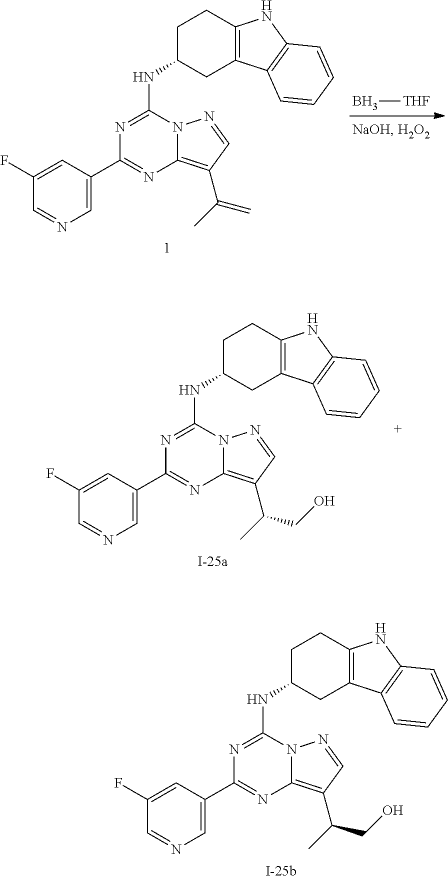

### Step 1: (2R)-2-[2-(5-Fluoro-3-pyridyl)-4-[[(3R)-2,3,4,9-tetrahydro-1H-carbazol-3-yl]amino]pyrazolo[1,5-a][1,3,5]triazin-8-yl]propan-1-ol (I-25a) and (2S)-2-[2-(5-Fluoro-3-pyridyl)-4-[[(3R)-2,3,4,9-tetrahydro-1H-carbazol-3-yl]amino]pyrazolo[1,5-a][1,3,5]triazin-8-yl]propan-1-ol (I-25b)

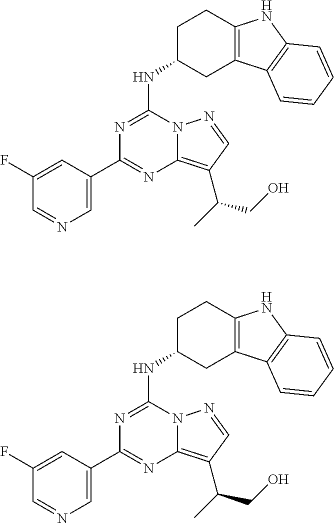 [SMILES: C[C@@H](CO)c1cnn2c(N[C@@H]3CCc4[nH]c5ccccc5c4C3)nc(-c3cncc(F)c3)nc12 ; C[C@H](CO)c1cnn2c(N[C@@H]3CCc4[nH]c5ccccc5c4C3)nc(-c3cncc(F)c3)nc12]

To a solution of (3R)—N-[2-(5-fluoro-3-pyridyl)-8-isopropenyl-pyrazolo[1,5-a][1,3,5]triazin-4-yl]-2,3,4,9-tetrahydro-1H-carbazol-3-amine (280 mg, 637.11 μmol, 1 eq) in THE (6 mL) was added BH₃.THF (1 M, 1.91 mL, 3 eq) at 0° C. The mixture was stirred at 0° C. for 2 h. Then NaOH (1 M, 3.65 mL, 5.73 eq) and H₂O₂ (4.31 g, 38.01 mmol, 3.65 mL, 30.0% purity, 59.66 eq) were added to the above mixture. The mixture was stirred at 25° C. for 3 h. The reaction mixture was quenched by addition aqueous solution of Na₂S203 (50 mL), extracted with EtOAc (20 mL×3). The combined organic layers were dried over Na₂SO₄, filtered and concentrated under reduced pressure to yield a residue which was purified by flash silica gel chromatography (from PE/EtOAc=100/1 to 1/1, TLC: PE/EtOAc=1/1, R_{f}=0.35) to yield a residue which was separated by SFC (column: DAICEL CHIRALPAK AD (250 mm*30 mm, 10 um); mobile phase: [0.1% NH₃H₂O MeOH]; B %: 50%-50%) followed by lyophilization to yield an enantiomer (38.56 mg, 83.95 umol, 13.18% yield, 99.6% purity, SFC: Rₜ=1.105 min, ee=99.0%, [(a]^{23.9}_{D}=−49.248, CHCl₃, c=0.052 g/100 mL) as a white solid. ¹H NMR (400 MHz, CD3OD) δ ppm 9.40 (s, 1H), 8.57-8.46 (m, 2H), 8.00 (s, 1H), 7.38 (d, J=7.6 Hz, 1H), 7.27 (d, J=7.8 Hz, 1H), 7.08-7.00 (m, 1H), 6.99-6.92 (m, 1H), 4.89 (m, 1H), 3.92-3.83 (m, 1H), 3.76 (dd, J=6.7, 10.6 Hz, 1H), 3.30-3.25 (m, 2H), 3.07-2.88 (m, 3H), 2.45-2.33 (m, 1H), 2.32-2.19 (m, 1H), 1.43 (d, J=7.1 Hz, 3H); ES-LCMS m/z 458.2 [M+H]⁺; and the other enantiomer (28.99 mg, 63.37 umol, 9.95% yield, 100% purity, SFC: Rₜ 1.388 min, ee=99.8%, [α]²⁴.OD=−61.368, CHCl₃, c=0.052 g/100 mL) as a white solid. ¹H NMR (400 MHz, CD3OD) δ ppm 9.40 (s, 1H), 8.56-8.46 (m, 2H), 8.00 (s, 1H), 7.38 (d, J=7.6 Hz, 1H), 7.27 (d, J=8.1 Hz, 1H), 7.08-7.00 (m, 1H), 6.99-6.92 (m, 1H), 4.89 (m, 1H), 3.88 (dd, J=6.4, 10.5 Hz, 1H), 3.76 (dd, J=6.6, 10.5 Hz, 1H), 3.30-3.25 (m, 2H), 3.08-2.88 (m, 3H), 2.44-2.34 (m, 1H), 2.32-2.19 (m, 1H), 1.43 (d, J=7.1 Hz, 3H); ES-LCMS m/z 458.2 [M+H]⁺.

### Example 27

### Synthesis of Compound I-26a and I-26b

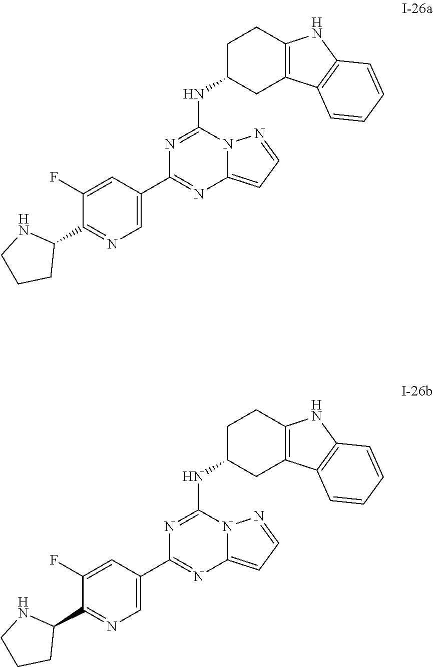 [SMILES: Fc1cc(-c2nc(N[C@@H]3CCc4[nH]c5ccccc5c4C3)n3nccc3n2)cnc1[C@@H]1CCCN1 ; Fc1cc(-c2nc(N[C@@H]3CCc4[nH]c5ccccc5c4C3)n3nccc3n2)cnc1[C@H]1CCCN1]

### Synthetic Scheme:

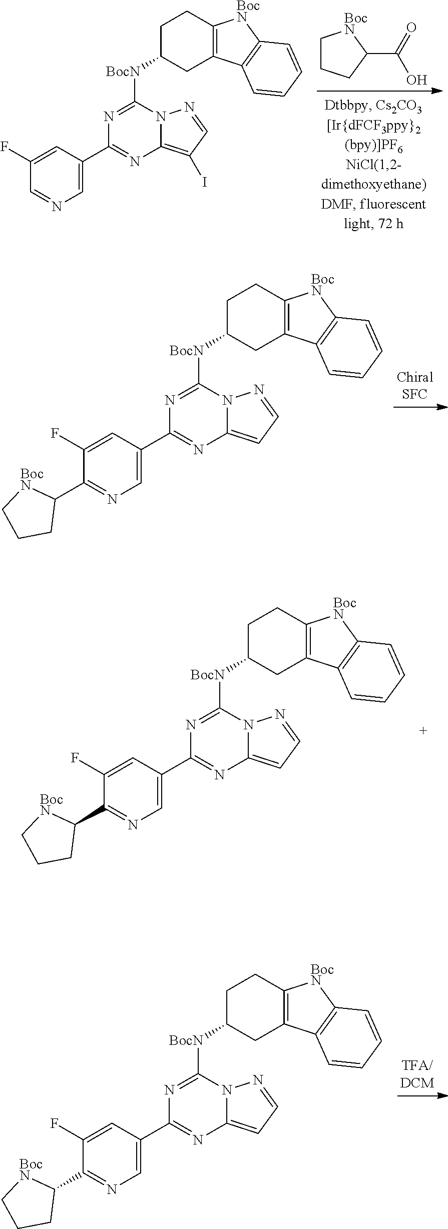

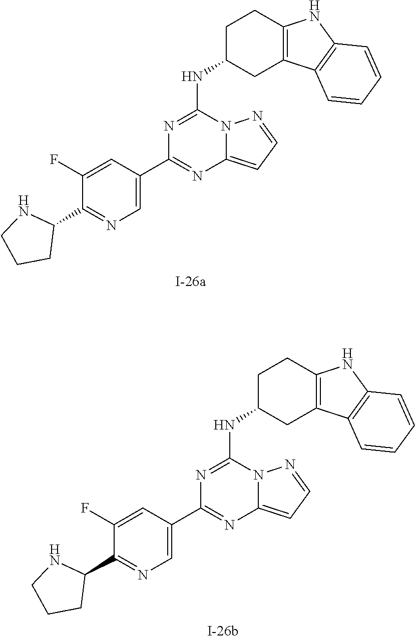 [SMILES: Fc1cc(-c2nc(N[C@@H]3CCc4[nH]c5ccccc5c4C3)n3nccc3n2)cnc1[C@@H]1CCCN1 ; Fc1cc(-c2nc(N[C@@H]3CCc4[nH]c5ccccc5c4C3)n3nccc3n2)cnc1[C@H]1CCCN1]

### Step 1: tert-Butyl (3R)-3-[tert-butoxycarbonyl-[2-[6-[(2S)-1-tert-butoxycarbonylpyrrolidin-2-yl]-5-fluoro-3-pyridyl]pyrazolo[1,5-a][1,3,5]triazin-4-yl]amino]-1,2,3,4-tetrahydrocarbazole-9-carboxylate & tert-butyl (3R)-3-[tert-butoxycarbonyl-[2-[6-[(2R)-1-tert-butoxycarbonylpyrrolidin-2-yl]-5-fluoro-3-pyridyl]pyrazolo[1,5-a][1,3,5]triazin-4-yl]amino]-1,2,3,4-tetrahydrocarbazole-9-carboxylate

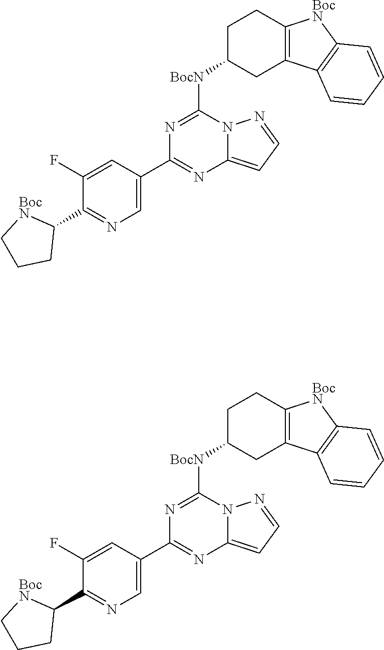 [SMILES: CC(C)(C)OC(=O)N1CCC[C@H]1c1ncc(-c2nc(N(C(=O)OC(C)(C)C)[C@@H]3CCc4c(c5ccccc5n4C(=O)OC(C)(C)C)C3)n3nccc3n2)cc1F ; CC(C)(C)OC(=O)N1CCC[C@@H]1c1ncc(-c2nc(N(C(=O)OC(C)(C)C)[C@@H]3CCc4c(c5ccccc5n4C(=O)OC(C)(C)C)C3)n3nccc3n2)cc1F]

Dichloronickel; 1,2-dimethoxyethane (11.74 mg, 53.42 μmol, 0.1 eq) and 4-tert-butyl-2-(4-tert-butyl-2-pyridyl)pyridine (14.34 mg, 53.42 μmol, 0.1 eq) were added into DMF (3 mL). The mixture was stirred at 50° C. under N₂ atmosphere until a green solution was obtained. [Ir{dFCF₃ppy}2(bpy)]PF₆ (5.99 mg, 5.34 μmol, 0.01 eq), Cs₂CO₃ (348.12 mg, 1.07 mmol, 2 eq), 1-tert-butoxycarbonylpyrrolidine-2-carboxylic acid (1.47 g, 6.81 mmol, 12.74 eq) and a solution of tert-butyl (3R)-3-[tert-butoxycarbonyl-[2-(5-fluoro-3-pyridyl)-8-iodo-pyrazolo[1,5-a][1,3,5]triazin-4-yl]amino]-1,2,3,4-tetrahydrocarbazole-9-carboxylate (400 mg, 534.22 μmol, 1 eq) in DMF (3 mL) were added into the above mixture under N₂ atmosphere. The resulting mixture was stirred and irradiated with a standard 72 W LED strip light bulb at 25° C. for 48 h. The reaction mixture was diluted with EtOAc (50 mL) and washed with 5% LiOH solution (30 mL×3). The organic layer was concentrated to yield a residue which was purified by flash silica gel chromatography (from PE/EtOAc=100/1 to 3/1, TLC: PE/EtOAc=3/1, R_{f}=0.45) and separated by SFC (column: DAICEL CHIRALPAK IC (250 mm*30 mm, 5 μm); mobile phase: [0.1% NH₃H₂O EtOH]; B %: 45%-45%) to yield an intermediate enantiomer (75 mg, 86.62 μmol, 16.2% yield, 88.8% purity, SFC: Rₜ=4.012 min) as a white solid. ¹H NMR (400 MHz, CDCl₃) δ ppm 9.44-9.33 (m, 1H), 8.31 (d, J=10.8 Hz, 1H), 8.26-8.22 (m, 1H), 8.11 (d, J=8.1 Hz, 1H), 7.40-7.31 (m, 1H), 7.24 (d, J=7.1 Hz, 1H), 7.22-7.16 (m, 1H), 6.76-6.69 (m, 1H), 5.32-5.13 (m, 1H), 4.99-4.85 (m, 1H), 3.78-3.51 (m, 3H), 3.37-3.12 (m, 3H), 2.50 (td, J=6.2, 12.0 Hz, 1H), 2.45-2.27 (m, 2H), 2.10 (dd, J=6.1, 11.7 Hz, 1H), 2.03-1.86 (m, 2H), 1.67 (s, 9H), 1.38 (t, J=9.0 Hz, 9H), 1.35-1.13 (m, 9H); ES-LCMS m/z 769.4 [M+H]⁺ and the other intermediate enantiomer (65 mg, 69.66 μmol, 13.04% yield, 82.4% purity, SFC: Rₜ=4.590 min) as a white solid. ¹H NMR (400 MHz, CDCl₃) δ ppm 9.45-9.33 (m, 1H), 8.31 (dd, J=1.5, 10.8 Hz, 1H), 8.27-8.15 (m, 1H), 8.14-8.08 (m, 1H), 7.39-7.31 (m, 1H), 7.26-7.22 (m, 1H), 7.21-7.15 (m, 1H), 6.76-6.62 (m, 1H), 5.31-5.13 (m, 1H), 4.99-4.87 (m, 1H), 3.76-3.66 (m, 3H), 3.38-3.10 (m, 3H), 2.62-2.49 (m, 1H), 2.36 (d, J=6.6 Hz, 2H), 2.10 (dd, J=6.2, 11.1 Hz, 1H), 2.03-1.86 (m, 2H), 1.68 (s, 9H), 1.38 (t, J=8.6 Hz, 9H), 1.27-1.23 (m, 4H), 1.21-1.16 (m, 5H); ES-LCMS m/z 769.4 [M+H]⁺.

### Step 2: (3R)—N-[2-[5-Fluoro-6-[(2S)-pyrrolidin-2-yl]-3-pyridyl]-8-[(2R)-pyrrolidin-2-yl]pyrazolo[1,5-a][1,3,5]triazin-4-yl]-2,3,4,9-tetrahydro-1H-carbazol-3-amine (I-26a)

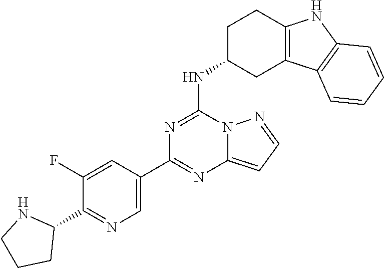 [SMILES: Fc1cc(-c2nc(N[C@@H]3CCc4[nH]c5ccccc5c4C3)n3nccc3n2)cnc1[C@@H]1CCCN1]

One of the intermediate enantiomers (75 mg, 70.99 μmol, 1 eq) was added to the mixture of TFA (2 mL) and DCM (6 mL). The reaction mixture was stirred at 25° C. for 1 h. The reaction mixture was concentrated to yield a residue which was purified by preparative HPLC (HCl condition; column: YMC-Actus Triart C18 100*30 mm*5 um; mobile phase: [water (0.05% HCl)−ACN]; B %: 25%-55%, 10 min). The desired fraction was lyophilized to yield an enantiomer (31.27 mg, 48.33 μmol, 68.1% yield, 100.0% purity, 3HCl, SFC: Rₜ=1.230 min, ee=99.6%, [α]^{25.6}_{D}=+96.138, MeOH, c=0.050 g/100 mL) as a white solid. ¹H NMR (400 MHz, CD3OD) δ ppm 9.43 (s, 1H), 8.57 (dd, J=1.5, 10.8 Hz, 1H), 8.11 (d, J=2.2 Hz, 1H), 7.38 (d, J=7.6 Hz, 1H), 7.29 (d, J=8.1 Hz, 1H), 7.05 (t, J=7.6 Hz, 1H), 6.99-6.94 (m, 1H), 6.53 (d, J=2.2 Hz, 1H), 5.13 (t, J=7.7 Hz, 1H), 4.84 (m, 1H), 3.62-3.53 (m, 1H), 3.51-3.45 (m, 1H), 3.35 (m, 1H), 3.15-2.90 (m, 3H), 2.68-2.57 (m, 1H), 2.39 (m, 1H), 2.36-2.26 (m, 1H), 2.25-2.07 (m, 3H); ES-LCMS m/z 469.2 [M+H]⁺.

### Step 3: (3R)—N-[2-[5-Fluoro-6-[(2R)-pyrrolidin-2-yl]-3-pyridyl]pyrazolo[1,5-a][1,3,5]triazin-4-yl]-2,3,4,9-tetrahydro-1H-carbazol-3-amine (I-26b)

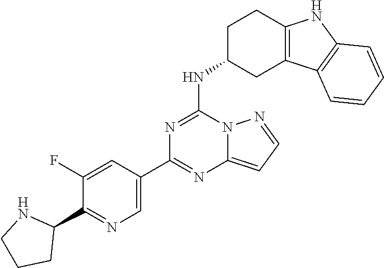 [SMILES: Fc1cc(-c2nc(N[C@@H]3CCc4[nH]c5ccccc5c4C3)n3nccc3n2)cnc1[C@H]1CCCN1]

The other intermediate enantiomer (65 mg, 69.66 μmol, 1 eq) was added to TFA (2 mL) and DCM (6 mL). The reaction mixture was stirred at 25° C. for 1 h. The reaction mixture was concentrated to yield a residue which was purified by preparative HPLC (HCl condition; column: YMC-Actus Triart C18 100*30 mm*5 um; mobile phase: [water (0.05% HCl)−ACN]; B %: 25%-55%, 10 min). The desired fraction was lyophilized to yield the other neantiomer (26.79 mg, 46.36 μmol, 66.6% yield, 100.0% purity, 3HCl, SFC: Rₜ=0.655 min, ee=100.0%, [α]^{25.6}_{D}=+23.602, MeOH, c=0.050 g/100 mL) as a white solid. ¹H NMR (400 MHz, CD3OD) δ ppm 9.43 (s, 1H), 8.57 (dd, J=1.5, 10.8 Hz, 1H), 8.11 (d, J=2.2 Hz, 1H), 7.38 (d, J=7.6 Hz, 1H), 7.29 (d, J=8.3 Hz, 1H), 7.09-7.01 (m, 1H), 7.00-6.93 (m, 1H), 6.53 (d, J=2.2 Hz, 1H), 5.14 (t, J=7.6 Hz, 1H), 4.83 (m, 1H), 3.61-3.54 (m, 1H), 3.51-3.45 (m, 1H), 3.35 (m, 1H), 3.16-2.90 (m, 3H), 2.69-2.56 (m, 1H), 2.38 (s, 1H), 2.36-2.25 (m, 1H), 2.25-2.07 (m, 3H); ES-LCMS m/z 469.2 [M+H]⁺.

### Example 28

### Synthesis of Compound I-27a

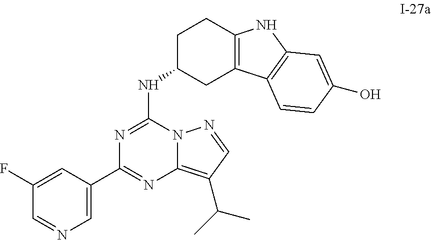 [SMILES: CC(C)c1cnn2c(N[C@@H]3CCc4[nH]c5cc(O)ccc5c4C3)nc(-c3cncc(F)c3)nc12]

### Synthetic Scheme:

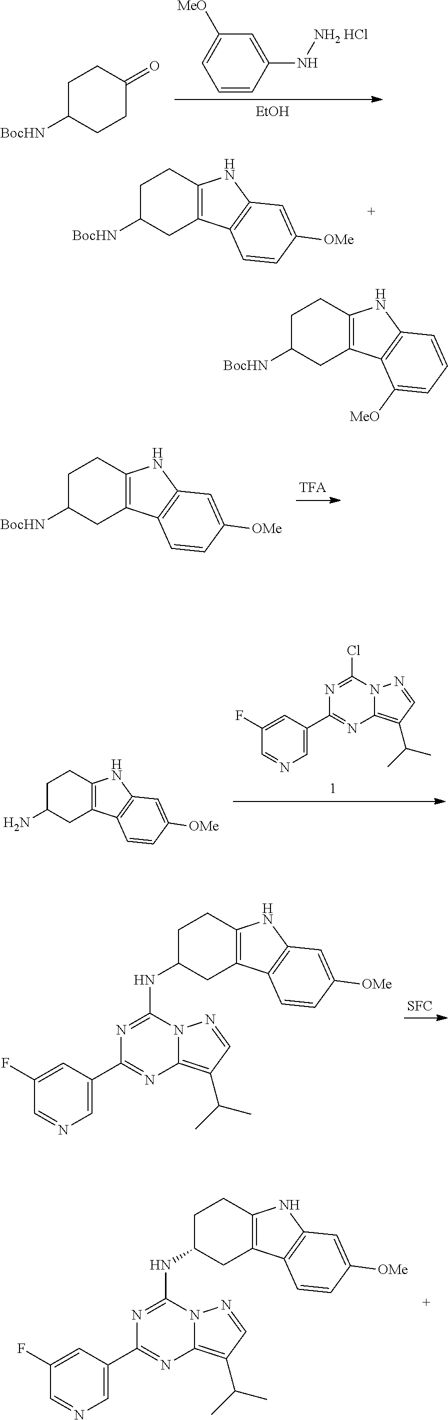

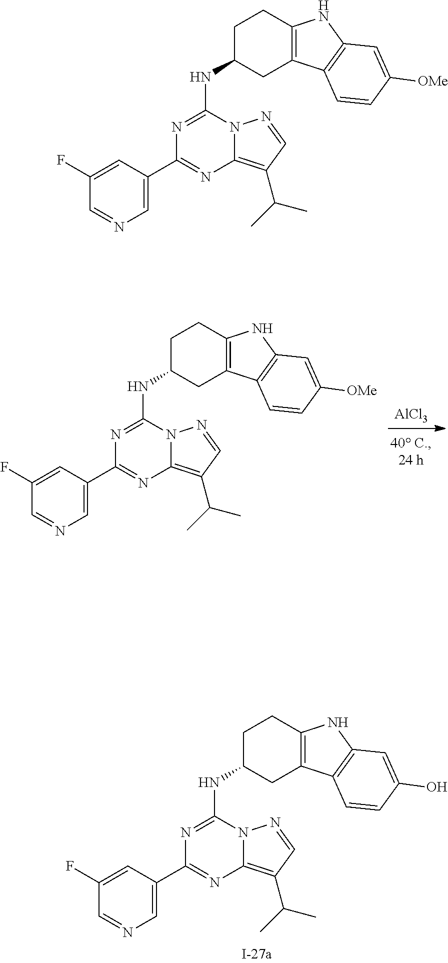

### Step 1: tert-Butyl N-(5-m ethoxy-2,3,4,9-tetrahydro-1H-carbazol-3-yl)carbamate & tert-Butyl N-(7-m ethoxy-2,3,4,9-tetrahydro-1H-carbazol-3-yl)carbamate

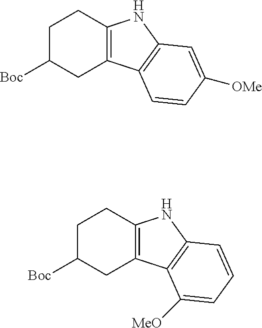 [SMILES: COc1ccc2c3c([nH]c2c1)CCC(C(=O)OC(C)(C)C)C3 ; COc1cccc2[nH]c3c(c12)CC(C(=O)OC(C)(C)C)CC3]

A mixture of tert-butyl N-(4-oxocyclohexyl)carbamate (2 g, 9.38 mmol, 2.00 mL, 1 eq) and (3-methoxyphenyl)hydrazine; hydrochloride (1.64 g, 9.38 mmol, 1 eq) in EtOH (40 mL) was stirred at 70° C. for 1 h. The reaction mixture was concentrated. The residue was purified by silica gel column chromatography (from pure PE to PE/EtOAc=3/1) to yield tert-butyl N-(5-methoxy-2,3,4,9-tetrahydro-1H-carbazol-3-yl)carbamate (360 mg, 1.10 mmol, 11.7% yield, 96.5% purity) as a yellow solid (TLC: PE/EtOAc=3/1, R_{f}=0.40). ¹H NMR (400 MHz, CDCl₃) δ ppm 7.72 (s, 1H), 7.05-6.99 (m, 1H), 6.89 (d, J=8.0 Hz, 1H), 6.46 (d, J=7.8 Hz, 1H), 4.71 (s, 1H), 4.08 (s, 1H), 3.88 (s, 3H), 3.31 (dd, J=5.3, 16.1 Hz, 1H), 2.88-2.74 (m, 3H), 2.14-2.04 (m, 1H), 1.98-1.89 (m, 1H), 1.47-1.43 (m, 9H); ES-LCMS m/z 261.1 [M−t-Bu+H]⁺. And tert-butyl N-(7-methoxy-2,3,4,9-tetrahydro-1H-carbazol-3-yl)carbamate (1.1 g, 3.37 mmol, 35.9% yield, 96.9% purity) as a yellow solid. ¹H NMR (400 MHz, CDCl₃) δ ppm 7.61 (s, 1H), 7.28 (d, J=8.6 Hz, 1H), 6.80 (d, J=2.2 Hz, 1H), 6.73 (dd, J=2.3, 8.7 Hz, 1H), 4.68 (m, 1H), 4.10 (m, 1H), 3.82 (s, 3H), 3.03 (dd, J=5.0, 15.3 Hz, 1H), 2.82-2.72 (m, 2H), 2.54 (dd, J=6.8, 15.2 Hz, 1H), 2.07 (d, J=6.4 Hz, 1H), 1.95 (d, J=5.6 Hz, 1H), 1.44 (s, 9H); ES-LCMS m/z 261.2 [M−t-Bu+H]⁺.

### Step 2: 7-Methoxy-2,3,4,9-tetrahydro-1H-carbazol-3-amine

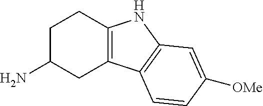 [SMILES: COc1ccc2c3c([nH]c2c1)CCC(N)C3]

To a solution of tert-butyl N-(7-methoxy-2,3,4,9-tetrahydro-1H-carbazol-3-yl)carbamate (250 mg, 765.66 μmol, 1 eq) in DCM (8 mL) was added TFA (3.08 g, 27.01 mmol, 2 mL, 35.28 eq). The reaction mixture was stirred at 20° C. for 0.5 h. The reaction mixture was concentrated to yield 7-methoxy-2,3,4,9-tetrahydro-1H-carbazol-3-amine (270 mg, 735.69 umol, 96.1% yield, 90.0% purity, TFA) as yellow oil which was used in the next step without purification. ¹H NMR (400 MHz, CDCl₃) δ ppm 7.38 (s, 2H), 7.29 (s, 1H), 7.19 (s, 1H), 6.83 (s, 1H), 6.77 (dd, J=2.1, 8.7 Hz, 1H), 3.85 (s, 3H), 3.14 (d, J=13.6 Hz, 1H), 2.88 (s, 3H), 2.27-2.04 (m, 3H); ES-LCMS m/z 217.2 [M+H]⁺.

### Step 3: (3R)—N-[2-(5-Fluoro-3-pyridyl)-8-isopropyl-pyrazolo[1,5-a][1,3,5]triazin-4-yl]-7-methoxy-2,3,4,9-tetrahydro-1H-carbazol-3-amine & (3S)—N-[2-(5-Fluoro-3-pyridyl)-8-isopropyl-pyrazolo[1,5-a][1,3,5]triazin-4-yl]-7-methoxy-2,3,4,9-tetrahydro-1H-carbazol-3-amine

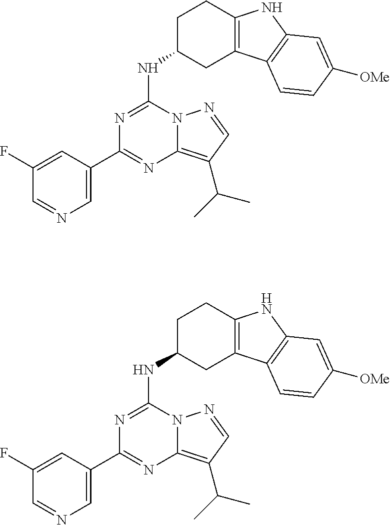 [SMILES: COc1ccc2c3c([nH]c2c1)CC[C@@H](Nc1nc(-c2cncc(F)c2)nc2c(C(C)C)cnn12)C3 ; COc1ccc2c3c([nH]c2c1)CC[C@H](Nc1nc(-c2cncc(F)c2)nc2c(C(C)C)cnn12)C3]

A mixture of 4-chloro-2-(5-fluoro-3-pyridyl)-8-isopropyl-pyrazolo[1,5-a][1,3,5]triazine (170 mg, 513.42 μmol, 1 eq), 7-methoxy-2,3,4,9-tetrahydro-1H-carbazol-3-amine (230.65 mg, 628.48 μmol, 1.22 eq, TFA) and DIEA (265.42 mg, 2.05 mmol, 357.71 μL, 4 eq) in CH₃CN (20 mL) was stirred at 70° C. for 2 h. The reaction mixture was concentrated to yield a residue which was purified by silica gel column chromatography (from PE/EtOAc=5/1 to 2/1, TLC: PE/EtOAc=3/1, R_{f}=0.2) then separated by chiral SFC (column: DAICEL CHIRALPAK AD-H (250 mm*30 mm, 5 um); mobile phase: [0.1% NH₁₃H₂OIPA]; B %: 40%-40%) to yield peak 1 (3R)—N-[2-(5-fluoro-3-pyridyl)-8-isopropyl-pyrazolo[1,5-a][1,3,5]triazin-4-yl]-7-methoxy-2,3,4,9-tetrahydro-1H-carbazol-3-amine (70 mg, 139.55 μmol, 27.2% yield, 94.0% purity, SFC: Rₜ=2.566 min) as a yellow solid. ¹H NMR (400 MHz, CDCl₃) δ ppm 9.51 (s, 1H), 8.55 (s, 1H), 8.44 (d, J=9.8 Hz, 1H), 7.84 (s, 1H), 7.73 (s, 1H), 7.33 (d, J=8.8 Hz, 1H), 6.86 (d, J=2.3 Hz, 1H), 6.78 (dd, J=2.3, 8.5 Hz, 1H), 6.70 (d, J=8.3 Hz, 1H), 4.94 (m, 1H), 3.86 (s, 3H), 3.36-3.27 (m, 2H), 3.00-2.86 (m, 3H), 2.38-2.26 (m, 2H), 1.41 (d, J=7.0 Hz, 6H); ES-LCMS m/z 472.3 [M+H]⁺. and peak 2 (3S)—N-[2-(5-fluoro-3-pyridyl)-8-isopropyl-pyrazolo[1,5-a][1,3,5]triazin-4-yl]-7-methoxy-2,3,4,9-tetrahydro-1H-carbazol-3-amine (110 mg, 228.62 μmol, 44.5% yield, 98.0% purity, Rₜ=3.445 min) as a yellow solid. ¹H NMR (400 MHz, CDCl₃) δ ppm 9.51 (s, 1H), 8.55 (d, J=2.8 Hz, 1H), 8.45 (td, J=2.2, 9.6 Hz, 1H), 7.84 (s, 1H), 7.73 (s, 1H), 7.33 (d, J=8.5 Hz, 1H), 6.86 (d, J=2.3 Hz, 1H), 6.78 (dd, J=2.3, 8.5 Hz, 1H), 6.71 (d, J=8.0 Hz, 1H), 4.93 (s, 1H), 3.86 (s, 3H), 3.36-3.25 (m, 2H), 3.03-2.87 (m, 3H), 2.40-2.25 (m, 2H), 1.40 (d, J=6.8 Hz, 6H); ES-LCMS m/z 472.3 [M+H]⁺.

### Step 4: (6R)-6-[[2-(5-Fluoro-3-pyridyl)-8-isopropyl-pyrazolo[1,5-a][1,3,5]triazin-4-yl]amino]-6,7,8,9-tetrahydro-5H-carbazol-2-ol (I-27a)

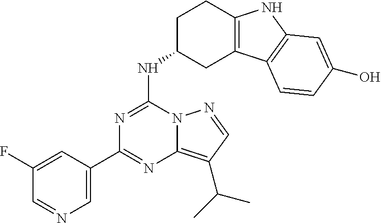 [SMILES: CC(C)c1cnn2c(N[C@@H]3CCc4[nH]c5cc(O)ccc5c4C3)nc(-c3cncc(F)c3)nc12]

To a solution of (3R)—N-[2-(5-fluoro-3-pyridyl)-8-isopropyl-pyrazolo[1,5-a][1,3,5]triazin-4-yl]-7-methoxy-2,3,4,9-tetrahydro-1H-carbazol-3-amine (70 mg, 139.55 μmol, 1 eq) in DCM (15 mL) was added AlCl₃ (1 g, 7.50 mmol, 409.84 μL, 53.74 eq). The reaction mixture was stirred at 40° C. for 12 h. The mixture was extracted with DCM (50 mL×3). The combined organic layers were washed with brine (50 mL), dried over Na₂SO₄, filtered and concentrated. The residue was purified by preparative HPLC (column: Xtimate C18 150*25 mm*5 um; mobile phase: [water (0.05% HCl)−ACN]; B %: 55%-85%, 8 min), followed by lyophilization to yield (6R)-6-[[2-(5-fluoro-3-pyridyl)-8-isopropyl-pyrazolo[1,5-a][1,3,5]triazin-4-yl]amino]-6,7,8,9-tetrahydro-5H-carbazol-2-ol (19.20 mg, 36.20 μmol, 25.9% yield, 100.0% purity, 2HCl, SFC: Rₜ=1.170 min, ee=97.588%, [α]^{25.6}_{D}=+5.799 (MeOH, c=0.051 g/100 mL)) as a yellow solid. ¹H NMR (400 MHz, CD3OD) δ ppm 9.44 (s, 1H), 8.72-8.65 (m, 2H), 8.00 (s, 1H), 7.19 (d, J=8.5 Hz, 1H), 6.74 (d, J=2.0 Hz, 1H), 6.56 (dd, J=2.3, 8.3 Hz, 1H), 4.78 (m, 1H), 3.30-3.22 (m, 2H), 3.01-2.85 (m, 3H), 2.41-2.33 (m, 1H), 2.30-2.20 (m, 1H), 1.42 (d, J=7.0 Hz, 6H); ES-LCMS m/z 458.3 [M+H]⁺.

### Example 29

### Synthesis of Compound I-27b

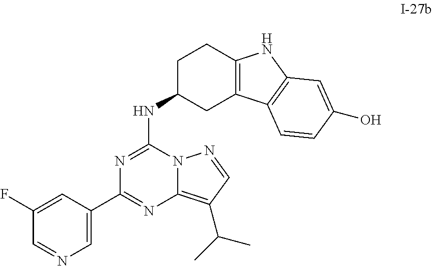 [SMILES: CC(C)c1cnn2c(N[C@H]3CCc4[nH]c5cc(O)ccc5c4C3)nc(-c3cncc(F)c3)nc12]

### Synthetic Scheme:

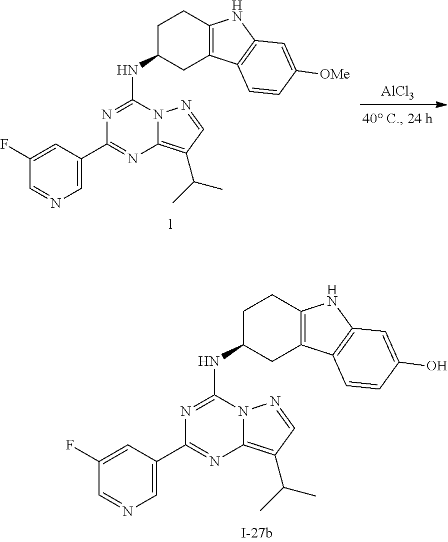

### Step 1: (6S)-6-[[2-(5-Fluoro-3-pyridyl)-8-isopropyl-pyrazolo[1,5-a][1,3,5]triazin-4-yl]amino]-6,7,8,9-tetrahydro-5H-carbazol-2-ol (I-27b)

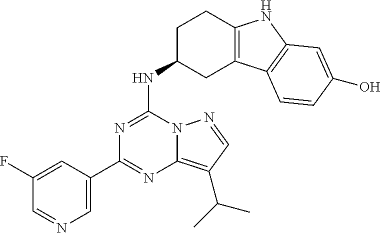 [SMILES: CC(C)c1cnn2c(N[C@H]3CCc4[nH]c5cc(O)ccc5c4C3)nc(-c3cncc(F)c3)nc12]

(3S)—N-[2-(5-fluoro-3-pyridyl)-8-isopropyl-pyrazolo[1,5-a][1,3,5]triazin-4-yl]-7-methoxy-2,3,4,9-tetrahydro-1H-carbazol-3-amine (90 mg, 187.05 μmol, 1 eq) in DCM (15 mL) was added AlCl₃ (1 g, 7.50 mmol, 409.84 μL, 40.09 eq). The reaction mixture was stirred at 40° C. for 12 h. The mixture was extracted with DCM (50 mL×3). The combined organic layers were washed with brine (50 mL), dried over Na₂SO₄, filtered and concentrated. The residue was purified by preparative HPLC (column: Xtimate C18 150*25 mm*5 um; mobile phase: [water (0.05% HCl)−ACN]; B %: 55%-85%, 8 min) followed by lyophilization to yield (6S)-6-[[2-(5-fluoro-3-pyridyl)-8-isopropyl-pyrazolo[1,5-a][1,3,5]triazin-4-yl]amino]-6,7,8,9-tetrahydro-5H-carbazol-2-ol (17.76 mg, 33.48 μmol, 17.9% yield, 100.0% purity, 2HCl, SFC: Rₜ=0.861 min, [α]^{25.6}_{D}=−19.229 (MeOH, c=0.051 g/100 mL)) as a yellow solid. ¹H NMR (400 MHz, CD3OD) δ ppm 9.45 (s, 1H), 8.73-8.66 (m, 2H), 8.00 (s, 1H), 7.19 (d, J=8.5 Hz, 1H), 6.74 (d, J=2.0 Hz, 1H), 6.56 (dd, J=2.1, 8.4 Hz, 1H), 4.82 (m, 1H), 3.30-3.22 (m, 2H), 3.03-2.85 (m, 3H), 2.36 (d, J=6.8 Hz, 1H), 2.30-2.21 (m, 1H), 1.43 (d, J=7.0 Hz, 6H); ES-LCMS m/z 458.3 [M+H]⁺.

### Example 30

### Synthesis of Compound I-29a, I-29b and I-29c

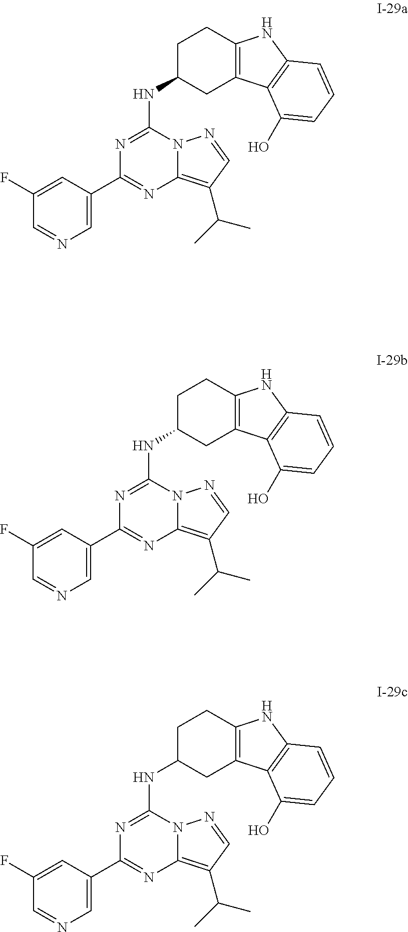 [SMILES: CC(C)c1cnn2c(N[C@H]3CCc4[nH]c5cccc(O)c5c4C3)nc(-c3cncc(F)c3)nc12 ; CC(C)c1cnn2c(N[C@@H]3CCc4[nH]c5cccc(O)c5c4C3)nc(-c3cncc(F)c3)nc12 ; CC(C)c1cnn2c(NC3CCc4[nH]c5cccc(O)c5c4C3)nc(-c3cncc(F)c3)nc12]

### Synthetic Scheme:

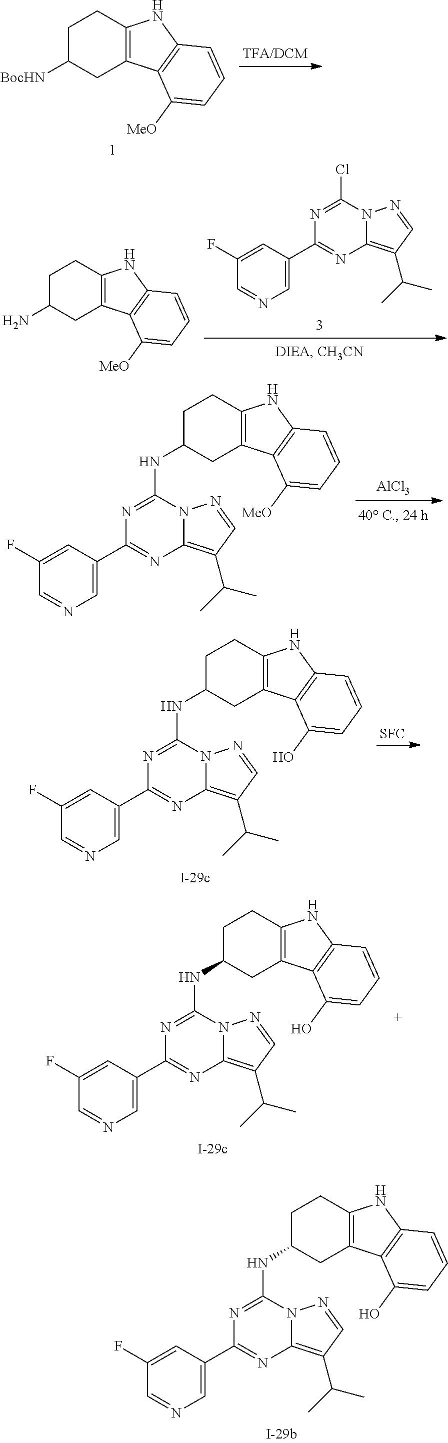

### Step 1: 5-Methoxy-2,3,4,9-tetrahydro-1H-carbazol-3-amine

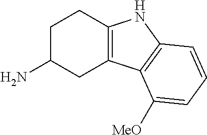 [SMILES: COc1cccc2[nH]c3c(c12)CC(N)CC3]

To a solution of tert-butyl N-(5-methoxy-2,3,4,9-tetrahydro-1H-carbazol-3-yl)carbamate (170 mg, 526.56 μmol, 1 eq) in DCM (8 mL) was added TFA (3.08 g, 27.01 mmol, 2 mL, 51.30 eq). The reaction mixture was stirred at 20° C. for 0.5 h. The reaction mixture was concentrated to yield 5-methoxy-2,3,4,9-tetrahydro-1H-carbazol-3-amine (180 mg, 511.17 μmol, 97.1% yield, 93.8% purity, TFA) as brown oil. The product was used in the next step without purification. ¹H NMR (400 MHz, CD3OD) δ ppm 6.99-6.93 (m, 1H), 6.90-6.87 (m, 1H), 6.46 (d, J=7.5 Hz, 1H), 3.87 (s, 3H), 3.64-3.59 (m, 1H), 3.49 (dd, J=5.1, 15.7 Hz, 1H), 3.01-2.90 (m, 3H), 2.30-2.23 (m, 1H), 2.06-2.01 (m, 1H); ES-LCMS m/z 217.3 [M+H]⁺.

### Step 2: N-[2-(5-Fluoro-3-pyridyl)-8-isopropyl-pyrazolo[1,5-a][1,3,5]triazin-4-yl]-5-methoxy-2,3,4,9-tetrahydro-1H-carbazol-3-amine

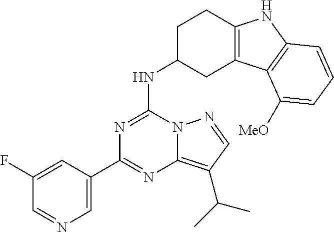 [SMILES: COc1cccc2[nH]c3c(c12)CC(Nc1nc(-c2cncc(F)c2)nc2c(C(C)C)cnn12)CC3]

A mixture of 4-chloro-2-(5-fluoro-3-pyridyl)-8-isopropyl-pyrazolo[1,5-a][1,3,5]triazine (120 mg, 362.41 μmol, 1 eq), 5-methoxy-2,3,4,9-tetrahydro-1H-carbazol-3-amine (153.14 mg, 434.90 μmol, 1.2 eq, TFA) and DIEA (187.35 mg, 1.45 mmol, 252.50 μL, 4 eq) in CH₃CN (10 mL) was stirred at 70° C. for 2 h. The reaction mixture was concentrated. The brown solid (200 mg) was added PE/EtOAc (1/1, 20 mL) and stirred at 20° C. for 1 h. The suspension was filtered and solid was collected, washed with PE/EtOAc (2/1, 5 mL×2), treated under vacuum to yield N-[2-(5-fluoro-3-pyridyl)-8-isopropyl-pyrazolo[1,5-a][1,3,5]triazin-4-yl]-5-methoxy-2,3,4,9-tetrahydro-1H-carbazol-3-amine (180 mg, 335.93 μmol, 92.7% yield, 88.0% purity) as a yellow solid. ¹H NMR (400 MHz, DMSO-d₆) δ ppm 10.78 (s, 1H), 9.38 (s, 1H), 8.99 (d, J=8.5 Hz, 1H), 8.82 (s, 2H), 8.70 (d, J=2.8 Hz, 1H), 8.47-8.40 (m, 1H), 8.15 (s, 1H), 6.40 (dd, J=1.8, 6.8 Hz, 1H), 4.78 (d, J=8.8 Hz, 1H), 3.75 (s, 3H), 3.61 (d, J=4.0 Hz, 2H), 3.13 (d, J=3.0 Hz, 2H), 2.83 (d, J=15.8 Hz, 1H), 2.16 (d, J=4.3 Hz, 2H), 1.38 (d, J=7.0 Hz, 6H); ES-LCMS m/z 472.3 [M+H]⁺.

### Step 3: (6S)-6-[[2-(5-Fluoro-3-pyridyl)-8-isopropyl-pyrazolo[1,5-a][1,3,5]triazin-4-yl]amino]-6,7,8,9-tetrahydro-5H-carbazol-4-ol (I-29a) & (6R)-6-[[2-(5-Fluoro-3-pyridyl)-8-isopropyl-pyrazolo[1,5-a][1,3,5]triazin-4-yl]amino]-6,7,8,9-tetrahydro-5H-carbazol-4-ol (I-29b)

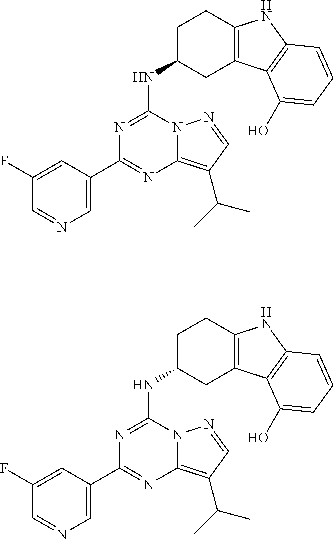 [SMILES: CC(C)c1cnn2c(N[C@H]3CCc4[nH]c5cccc(O)c5c4C3)nc(-c3cncc(F)c3)nc12 ; CC(C)c1cnn2c(N[C@@H]3CCc4[nH]c5cccc(O)c5c4C3)nc(-c3cncc(F)c3)nc12]

To a solution of N-[2-(5-fluoro-3-pyridyl)-8-isopropyl-pyrazolo[1,5-a][1,3,5]triazin-4-yl]-5-methoxy-2,3,4,9-tetrahydro-1H-carbazol-3-amine (170 mg, 317.27 μmol, 1 eq) in DCM (20 mL) was added AlCl₃ (2 g, 15.00 mmol, 819.67 μL, 47.28 eq). The reaction mixture was stirred at 40° C. for 12 h. The reaction mixture was extracted with DCM (50 mL×3). The combined organic layers were washed with brine (50 mL), dried over Na₂SO₄, filtered and concentrated to yield a residue which was purified by preparative HPLC (column: Xtimate C18 150*25 mm*5 um; mobile phase: [water (0.05% HCl)−ACN]; B %: 55%-85%, 8 min), then separated by chiral SFC (column: DAICEL CHIRALPAK AD (250 mm*30 mm, 10 um); mobile phase: [0.1% NH₃.H₂OEtOH]; B %: 50%-50%) to yield an enantiomer (15.72 mg, 34.36 μmol, 10.8% yield, 100.0% purity, SFC: Rₜ=1.284 min, [α]^{26.1}_{D}=−19.164 (MeOH, c=0.022 g/100 mL)) as a yellow solid. ¹H NMR (400 MHz, CD3OD) δ ppm 9.42 (s, 1H), 8.56-8.49 (m, 2H), 7.98 (s, 1H), 6.85-6.78 (m, 2H), 6.33 (dd, J=1.8, 6.8 Hz, 1H), 4.80 (m, 1H), 3.62 (dd, J=5.3, 15.8 Hz, 1H), 3.30-3.26 (m, 1H), 3.16 (dd, J=8.7, 15.4 Hz, 1H), 3.03-2.90 (m, 2H), 2.36 (d, J=6.5 Hz, 1H), 2.28-2.19 (m, 1H), 1.42 (d, J=7.0 Hz, 6H); ES-LCMS m/z 458.3 [M+H]⁺; and the other enantiomer (15.15 mg, 33.11 μmol, 10.4% yield, 100.0% purity, SFC: Rₜ=1.853 min, [α]^{25.8}_{D}=+16.454 (MeOH, c=0.007 g/100 mL)) as a yellow solid. ¹H NMR (400 MHz, CD3OD) δ ppm 9.42 (s, 1H), 8.56-8.49 (m, 2H), 7.98 (s, 1H), 6.86-6.78 (m, 2H), 6.33 (dd, J=1.6, 6.7 Hz, 1H), 4.80 (m, 1H), 3.62 (dd, J=5.0, 15.3 Hz, 1H), 3.30-3.25 (m, 1H), 3.16 (dd, J=8.5, 15.8 Hz, 1H), 3.06-2.89 (m, 2H), 2.36 (d, J=4.8 Hz, 1H), 2.29-2.18 (m, 1H), 1.42 (d, J=6.8 Hz, 6H); ES-LCMS m/z 458.2 [M+H]⁺.

### Example 31

### Synthesis of Compound I-30

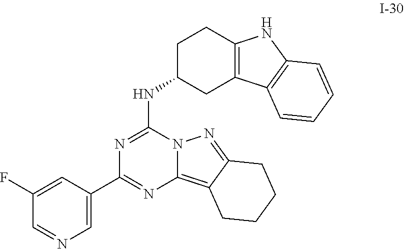 [SMILES: Fc1cncc(-c2nc(N[C@@H]3CCc4[nH]c5ccccc5c4C3)n3nc4c(c3n2)CCCC4)c1]

### Synthetic Scheme:

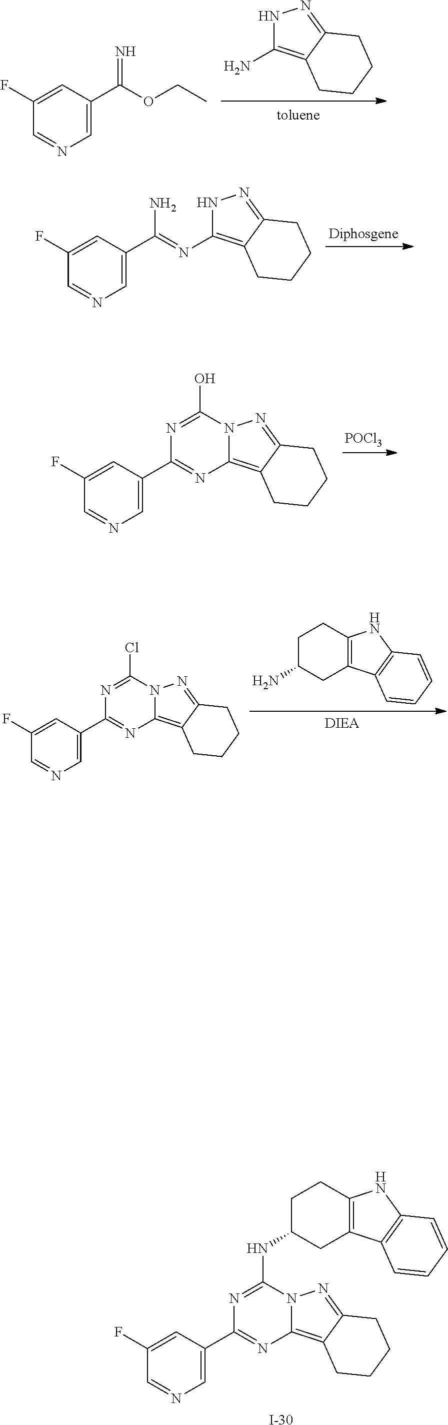

### Step 1: 5-Fluoro-N′-(4,5,6,7-tetrahydro-2H-indazol-3-yl)pyridine-3-carboxamidine

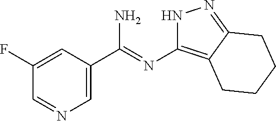 [SMILES: N/C(=N\c1[nH]nc2c1CCCC2)c1cncc(F)c1]

To a stirred solution of ethyl 5-fluoropyridine-3-carboximidate (100 mg, 564.91 μmol, 1 eq) in toluene (5 mL) was added 4,5,6,7-tetrahydro-2H-indazol-3-amine (98.09 mg, 564.91 μmol, 1 eq, HCl). The reaction mixture was stirred at 120° C. for 12 h under N₂ atmosphere. The reaction mixture was cooled to 0° C. under ice-water bath and filtered. The filtered cake was concentrated to yield 5-fluoro-N′-(4,5,6,7-tetrahydro-2H-indazol-3-yl)pyridine-3-carboxamidine (139 mg, 482.49 μmol, 85.4% yield, 90.0% purity) as a yellow solid which was used in the next step without further purification. ¹H NMR (400 MHz, DMSO-d₆) δ ppm 12.08 (s, 1H), 10.62 (s, 1H), 10.17 (s, 1H), 8.97-8.84 (m, 2H), 8.31 (td, J=2.3, 9.0 Hz, 1H), 2.60 (t, J=5.7 Hz, 2H), 2.49 (m, 2H), 1.70 (dd, J=6.7, 13.6 Hz, 4H); ES-LCMS m/z 260.1 [M+H]⁺.

### Step 2: 2-(5-Fluoro-3-pyridyl)-7,8,9,10-tetrahydro-[1,3,5]triazino[1,2-b]indazol-4-ol

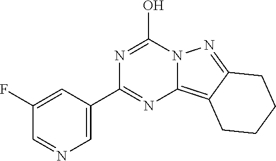 [SMILES: Oc1nc(-c2cncc(F)c2)nc2c3c(nn12)CCCC3]

To a stirred solution of 5-fluoro-N′-(4,5,6,7-tetrahydro-2H-indazol-3-yl)pyridine-3-carboxamidine (139 mg, 482.49 μmol, 1 eq) in 1,4-dioxane (2.5 mL) and THE (2.5 mL) was added triphosgene (114.54 mg, 578.98 μmol, 69.84 μL, 1.2 eq). The reaction mixture was stirred at 80° C. for 12 h. The reaction mixture was concentrated to yield 2-(5-fluoro-3-pyridyl)-7,8,9,10-tetrahydro-[1,3,5]triazino[1,2-b]indazol-4-ol (135 mg, crude) as yellow oil which was used in the next step without further purification. ¹H NMR (400 MHz, CD3OD) δ ppm 9.14 (s, 1H), 8.79 (d, J=2.8 Hz, 1H), 8.41 (td, J=2.1, 9.3 Hz, 1H), 2.82 (t, J=6.1 Hz, 2H), 2.76 (t, J=6.1 Hz, 2H), 1.87 (dd, J=8.7, 15.2 Hz, 4H); ES-LCMS m/z 286.1 [M+H]⁺.

### Step 3: 4-Chloro-2-(5-fluoro-3-pyridyl)-7,8,9,10-tetrahydro-[1,3,5]triazino[1,2-b]indazole

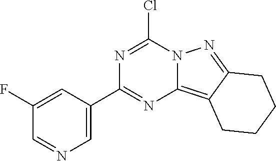 [SMILES: Fc1cncc(-c2nc(Cl)n3nc4c(c3n2)CCCC4)c1]

To a stirred solution of 2-(5-fluoro-3-pyridyl)-7,8,9,10-tetrahydro-[1,3,5]triazino[1,2-b] indazol-4-ol (135 mg, 473.23 μmol, 1 eq) in toluene (6 mL) was added DIEA (183.48 mg, 1.42 mmol, 247.28 μL, 3 eq) and POCl₃ (742.50 mg, 4.84 mmol, 450.00 μL, 10.23 eq). The reaction mixture was stirred at 120° C. for 2 h. The reaction mixture was concentrated to yield 4-chloro-2-(5-fluoro-3-pyridyl)-7,8,9,10-tetrahydro-[1,3,5]triazino[1,2-b]indazole (150 mg, crude, 2HCl) as brown oil which was used in the next step without further purification. ES-LCMS m/z 304.0, 306.1 [M+H]⁺.

### Step 4: (3R)—N-[8-Isopropyl-2-(1,2,4-triazol-1-yl)pyrazolo[1,5-a][1,3,5]triazin-4-yl]-2,3,4,9-tetrahydro-1H-carbazol-3-amine (I-30)

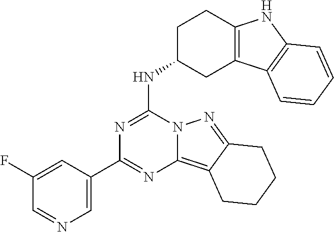 [SMILES: Fc1cncc(-c2nc(N[C@@H]3CCc4[nH]c5ccccc5c4C3)n3nc4c(c3n2)CCCC4)c1]

To a solution of 4-chloro-2-(5-fluoro-3-pyridyl)-7,8,9,10-tetrahydro-[1,3,5]triazino [1,2-b]indazole (150 mg, 398.25 μmol, 1 eq, 2HCl) in ACN (10 mL) was added DIEA (257.35 mg, 1.99 mmol, 346.84 μL, 5 eq) and (3R)-2,3,4,9-tetrahydro-1H-carbazol-3-amine (74.18 mg, 398.25 μmol, 1 eq). The mixture was stirred at 60° C. for 2 h. The reaction mixture was concentrated to yield a residue which was purified by preparative HPLC (HCl condition; column: DuraShell 150*25 mm*5 um; mobile phase: [water (0.05% HCl)−ACN]; B %: 59%-89%, 8 min). The desired fraction was lyophilized to yield 2-(5-fluoro-3-pyridyl)-N-[(3R)-2,3,4,9-tetrahydro-1H-carbazol-3-yl]-7,8,9,10-tetrahydro-[1,3,5]triazino[1,2-b]indazol-4-amine (60.53 mg, 114.98 μmol, 28.9% yield, 100.0% purity, 2HCl, [α]^{30.4}_{D}=+8.157 (DMSO, c=0.108 g/100 mL) as a yellow solid. ¹H NMR (400 MHz, CD3OD) δ ppm 9.40 (s, 1H), 8.61-8.54 (m, 2H), 7.38 (d, J=7.6 Hz, 1H), 7.28 (d, J=8.1 Hz, 1H), 7.04 (t, J=7.1 Hz, 1H), 6.99-6.93 (m, 1H), 4.84 (m, 1H), 3.28 (d, J=4.4 Hz, 1H), 3.11-2.88 (m, 3H), 2.81 (td, J=5.9, 11.7 Hz, 4H), 2.43-2.20 (m, 2H), 1.91 (dd, J=7.1, 13.9 Hz, 4H); ES-LCMS m/z 454.2 [M+H]⁺.

### Example 32

### Synthesis of Compound I-31

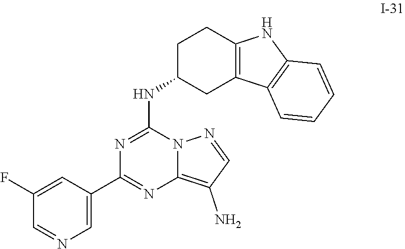 [SMILES: Nc1cnn2c(N[C@@H]3CCc4[nH]c5ccccc5c4C3)nc(-c3cncc(F)c3)nc12]

### Synthetic Scheme:

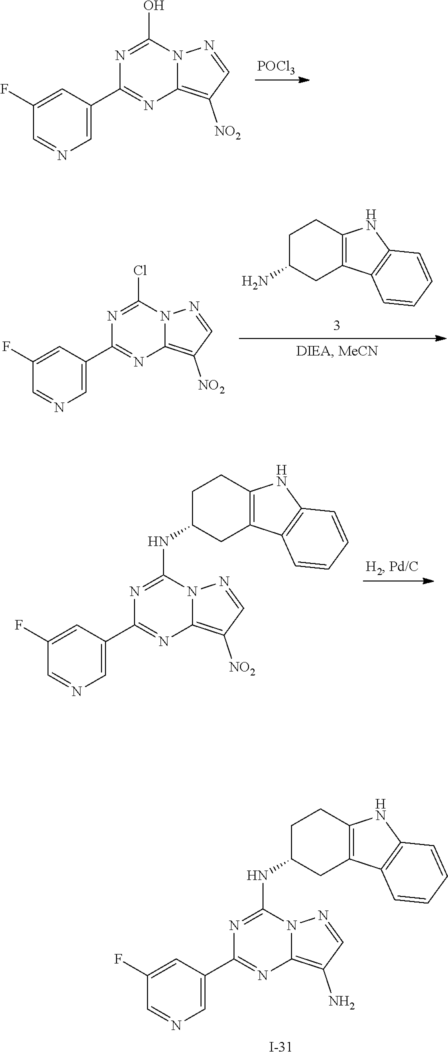

### Step 1: 4-Chloro-2-(5-fluoro-3-pyridyl)-8-nitro-pyrazolo[1,5-a][1,3,5]triazine

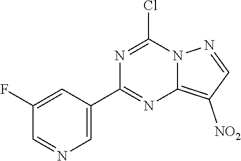 [SMILES: O=[N+]([O-])c1cnn2c(Cl)nc(-c3cncc(F)c3)nc12]

To a solution of 2-(5-fluoro-3-pyridyl)-8-nitro-pyrazolo[1,5-a][1,3,5]triazin-4-ol (500 mg, 1.81 mmol, 1 eq) in toluene (10 mL) was added POCl₃ (33.00 g, 215.22 mmol, 20 mL, 118.88 eq) and DIEA (701.94 mg, 5.43 mmol, 946.01 μL, 3 eq). The mixture was stirred at 120° C. for 12 h. The reaction mixture was concentrated under reduced pressure to yield 4-chloro-2-(5-fluoro-3-pyridyl)-8-nitro-pyrazolo[1,5-a][1,3,5]triazine (533.39 mg, crude) as a brown solid which was used in the next step without further purification. ES-LCMS m/z 295.0, 297.0 [M+H]⁺.

### Step 2: (3R)—N-[2-(5-Fluoro-3-pyridyl)-8-nitro-pyrazolo[1,5-a][1,3,5]triazin-4-yl]-2,3,4,9-tetrahydro-1H-carbazol-3-amine)

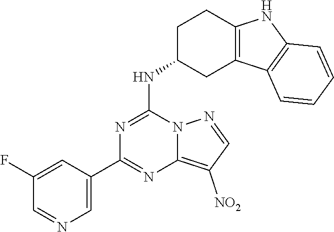 [SMILES: O=[N+]([O-])c1cnn2c(N[C@@H]3CCc4[nH]c5ccccc5c4C3)nc(-c3cncc(F)c3)nc12]

To a solution of 4-chloro-2-(5-fluoro-3-pyridyl)-8-nitro-pyrazolo[1,5-a][1,3,5]triazine (300 mg, 1.02 mmol, 1 eq) in CH₃CN (30 mL) was added DIEA (526.40 mg, 4.07 mmol, 709.43 μL, 4 eq) and (3R)-2,3,4,9-tetrahydro-1H-carbazol-3-amine (189.65 mg, 1.02 mmol, 1 eq). The mixture was stirred at 60° C. for 3 h. The reaction mixture was concentrated under reduced pressure to yield a residue which was purified by flash silica gel chromatography (from PE/EtOAC=100/1 to 1/1, TLC: PE/EtOAc=1/1, R_{f}=0.3) to yield (3R)—N-[2-(5-fluoro-3-pyridyl)-8-nitro-pyrazolo[1,5-a][1,3,5]triazin-4-yl]-2,3,4,9-tetrahydro-1H-carbazol-3-amine (100 mg, 225.01 μmol, 22.1% yield, 100.0% purity) as a yellow solid. ¹H NMR (400 MHz, CDCl₃) δ ppm 9.59 (s, 1H), 8.66 (d, J=2.7 Hz, 1H), 8.60-8.53 (m, 2H), 7.96 (s, 1H), 7.48 (d, J=7.8 Hz, 1H), 7.36 (d, J=8.1 Hz, 1H), 7.21 (t, J=7.1 Hz, 1H), 7.16-7.10 (m, 1H), 7.02 (s, 1H), 5.03 (m, 1H), 3.40 (dd, J=4.9, 15.7 Hz, 1H), 3.11-2.93 (m, 3H), 2.44-2.33 (m, 2H); ES-LCMS m/z 445.1 [M+H]⁺.

### Step 3: 2-(5-Fluoro-3-pyridyl)-N4-[(3R)-2,3,4,9-tetrahydro-1H-carbazol-3-yl]pyrazolo[1,5-a][1,3,5]triazine-4,8-diamine (I-31)

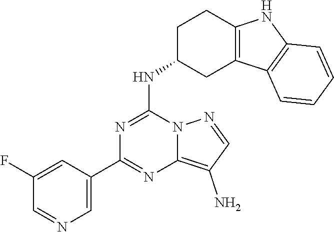 [SMILES: Nc1cnn2c(N[C@@H]3CCc4[nH]c5ccccc5c4C3)nc(-c3cncc(F)c3)nc12]

A mixture of (3R)—N-[2-(5-fluoro-3-pyridyl)-8-nitro-pyrazolo[1,5-a][1,3,5]triazin-4-yl]-2,3,4,9-tetrahydro-1H-carbazol-3-amine (60 mg, 135.01 μmol, 1 eq), Zn (353.13 mg, 5.40 mmol, 40 eq) and NH₄Cl (288.87 mg, 5.40 mmol, 40 eq) in H₂O (5 mL) and EtOH (20 mL) was degassed and purged with N₂ for 3 times, the mixture was stirred at 25° C. for 12 h under N₂ atmosphere. The reaction mixture was filtered and the filtrate was concentrated under reduced pressure to yield a residue which was purified by preparative HPLC (column: YMC-Actus Triart C18 100*30 mm*5 um; mobile phase: [water (0.05% HCl)−ACN]; B %: 30%-58%, 9 min), followed by lyophilization to yield 2-(5-fluoro-3-pyridyl)-N4-[(3R)-2,3,4,9-tetrahydro-1H-carbazol-3-yl]pyrazolo[1,5-a][1,3,5]triazine-4,8-diamine (17.03 mg, 39.37 μmol, 29.2% yield, 95.8% purity, [α]^{29.6}_{D}=+20.592 (DMSO, c=0.038 g/100 mL) as a brown solid. ¹H NMR (400 MHz, CD3OD) δ ppm 9.48 (s, 1H), 8.70-8.61 (m, 2H), 8.24 (s, 1H), 7.37 (d, J=7.6 Hz, 1H), 7.28 (d, J=7.8 Hz, 1H), 7.04 (t, J=7.5 Hz, 1H), 6.99-6.92 (m, 1H), 4.95 (m, 1H), 3.28 (s, 1H), 3.16-2.90 (m, 3H), 2.40 (s, 1H), 2.35-2.21 (in, 1H); ES-LCMS m/z 415.2 [M+H]⁺.

### Example 33

### Synthesis of Compound I-32a, I-32b and I-32c

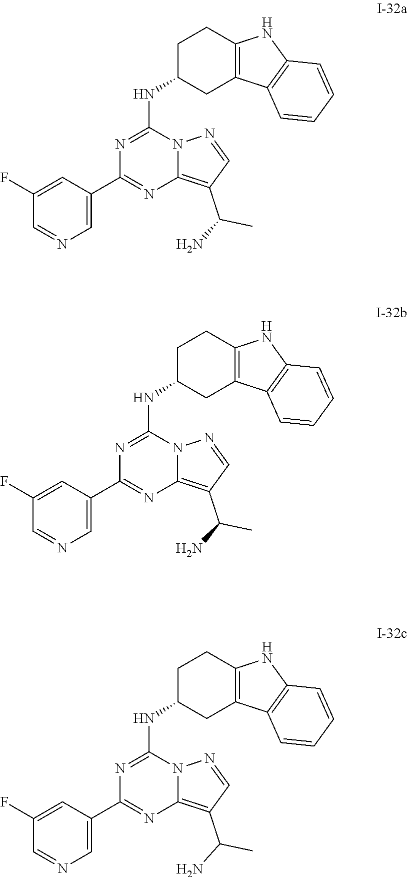 [SMILES: C[C@H](N)c1cnn2c(N[C@@H]3CCc4[nH]c5ccccc5c4C3)nc(-c3cncc(F)c3)nc12 ; C[C@@H](N)c1cnn2c(N[C@@H]3CCc4[nH]c5ccccc5c4C3)nc(-c3cncc(F)c3)nc12 ; CC(N)c1cnn2c(N[C@@H]3CCc4[nH]c5ccccc5c4C3)nc(-c3cncc(F)c3)nc12]

### Synthetic Scheme:

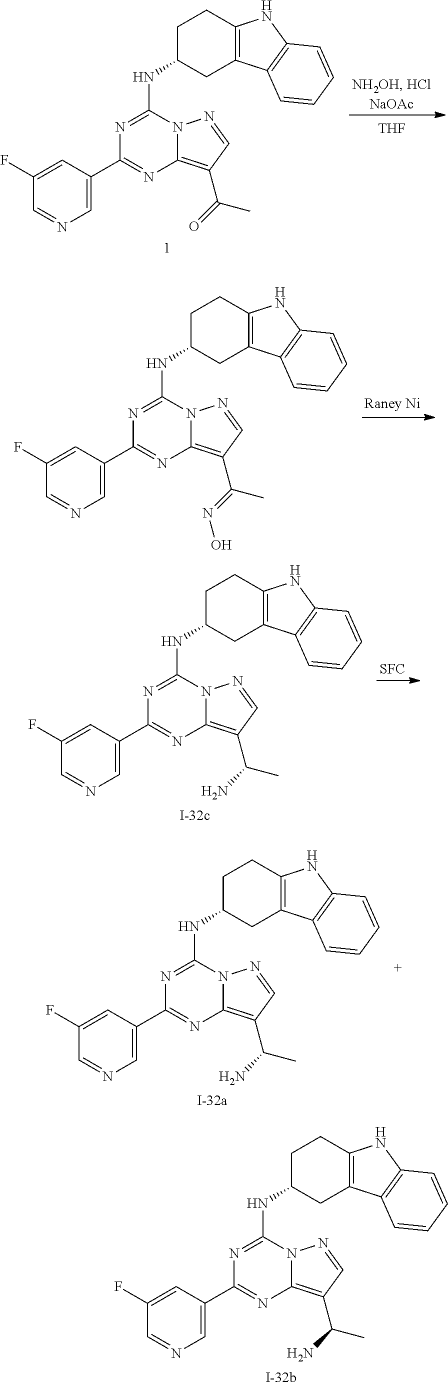

### Step 1: 1-[2-(5-Fluoro-3-pyridyl)-4-[[(3R)-2,3,4,9-tetrahydro-1H-carbazol-3-yl]amino]pyrazolo[1,5-a][1,3,5]triazin-8-yl]ethanone oxime

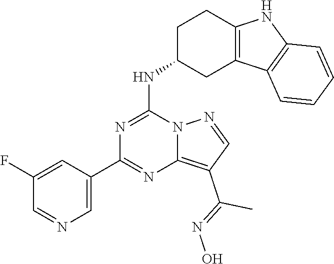 [SMILES: C/C(=N\O)c1cnn2c(N[C@@H]3CCc4[nH]c5ccccc5c4C3)nc(-c3cncc(F)c3)nc12]

To a solution of 1-[2-(5-fluoro-3-pyridyl)-4-[[(3R)-2,3,4,9-tetrahydro-1H-carbazol-3-yl] amino]pyrazolo[1,5-a][1,3,5]triazin-8-yl]ethanone (200 mg, 453.04 μmol, 1 eq) in THE (20 mL) was added NH₂OH HCl (37.78 mg, 543.65 μmol, 1.2 eq) and NaOAc (55.75 mg, 679.56 μmol, 1.5 eq). The mixture was stirred at 60° C. for 12 h under N₂ atmosphere. The mixture was concentrated and water (20 mL) was added. The mixture was extracted with EtOAc (30 mL×3). The combined organic layers were washed with brine (20 mL), dried over Na₂SO₄, filtered and concentrated to yield 1-[2-(5-fluoro-3-pyridyl)-4-[[(3R)-2,3,4,9-tetrahydro-1H-carbazol-3-yl]amino]pyrazolo[1,5-a][1,3,5]triazin-8-yl]ethanone oxime (200 mg, 413.17 μmol, 91.2% yield, 94.3% purity) as a yellow solid which was used in the next step without further purification. ¹H NMR (400 MHz, CD3OD) δ ppm 9.38 (s, 1H), 8.56-8.52 (m, 1H), 8.51-8.41 (m, 1H), 8.30 (s, 1H), 7.37 (d, J=7.8 Hz, 1H), 7.26 (d, J=8.1 Hz, 1H), 7.06-6.99 (m, 1H), 6.97-6.89 (m, 1H), 4.84-4.81 (m, 1H), 3.28 (d, J=5.1 Hz, 1H), 3.09-2.88 (m, 3H), 2.59-2.51 (m, 3H), 2.42-2.20 (m, 2H); ES-LCMS m/z 457.2 [M+H]⁺.

### Step 2: (3R)—N-[8-[(1S)-1-Aminoethyl]-2-(5-fluoro-3-pyridyl)pyrazolo[1,5-a][1,3,5]triazin-4-yl]-2,3,4,9-tetrahydro-1H-carbazol-3-amine (I-32a) & (3R)—N-[8-[(1R)-1-aminoethyl]-2-(5-fluoro-3-pyridyl)pyrazolo[1,5-a][1,3,5]triazin-4-yl]-2,3,4,9-tetrahydro-1H-carbazol-3-amine (I-32b)

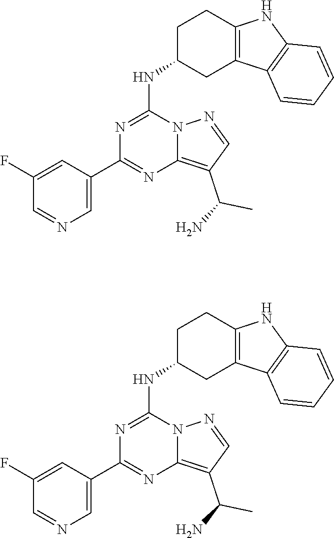 [SMILES: C[C@H](N)c1cnn2c(N[C@@H]3CCc4[nH]c5ccccc5c4C3)nc(-c3cncc(F)c3)nc12 ; C[C@@H](N)c1cnn2c(N[C@@H]3CCc4[nH]c5ccccc5c4C3)nc(-c3cncc(F)c3)nc12]

A mixture of 1-[2-(5-fluoro-3-pyridyl)-4-[[(3R)-2,3,4,9-tetrahydro-1H-carbazol-3-yl]amino]pyrazolo[1,5-a][1,3,5]triazin-8-yl]ethanone oxime (80 mg, 165.27 μmol, 1 eq), Raney-Ni (100 mg, need to remove water) and NH₃/MeOH (7 M, 2.40 mL) in MeOH (30 mL) was stirred under H₂ (15 Psi) at 25° C. for 3 h. The reaction mixture was filtered. The filtrate was concentrated under reduced pressure to yield a residue which was separated by chiral SFC (column: Phenomenex-Amylose-1 (250 mm*30 mm, 5 um); mobile phase: [0.1% NH₃.H₂OEtOH]; B %: 40%-40%) to yield Peak 1 and Peak 2. One of the peaks was concentrated under reduced pressure. The residue was purified by preparative HPLC (column: Boston Prime C18 150*30 mm Sum; mobile phase: [water (0.05% ammonia hydroxide v/v)−ACN]; B %: 43%-73%, 9 min). The desired fraction was lyophilized to yield a residue which was purified by preparative TLC (DCM/MeOH=8/1, R_{f}=0.30) to yield an enantiomer (14.53 mg, 32.84 μmol, 19.9% yield, 100.0% purity, SFC: Rₜ=2.832, ee=98.162%, [α]^{28.9}_{D}=−32.128 (MeOH, c=0.020 g/100 mL)) as a yellow solid. ¹H NMR (400 MHz, CD3OD) δ ppm 9.41 (s, 1H), 8.53 (d, J=2.4 Hz, 2H), 8.08 (s, 1H), 7.36 (d, J=7.6 Hz, 1H), 7.26 (d, J=8.0 Hz, 1H), 7.02 (t, J=7.6 Hz, 1H), 6.97-6.92 (m, 1H), 4.80-4.68 (m, 1H), 4.48 (d, J=6.8 Hz, 1H), 3.28-3.18 (m, 1H), 3.09-2.87 (m, 3H), 2.37 (s, 1H), 2.26 (d, J=5.2 Hz, 1H), 1.59 (d, J=6.4 Hz, 3H); ES-LCMS m/z 426.2 [M−NH₂]⁺. The other of these peaks was concentrated under reduced pressure to yield a residue which was separated by chiral SFC (column: Phenomenex-Amylose-1 (250 mm*30 mm, 5 um); mobile phase: [0.1% NH₃.H₂OEtOH]; B %: 40%-40%). The desired fraction was concentrated under reduced pressure to yield a residue which was purified by preparative TLC (DCM/MeOH=8/1, R_{f}=0.30) to yield the other enantiomer (4.48 mg, 9.70 μmol, 5.9% yield, 95.8% purity, SFC: Rₜ=3.640, ee=96.1388%, [α]^{29.4}_{D}=−24.575 (MeOH, c=0.006 g/100 mL)) as a yellow solid. ¹H NMR (400 MHz, CD3OD) δ ppm 9.40 (s, 1H), 8.53 (s, 2H), 8.07 (s, 1H), 7.36 (d, J=7.6 Hz, 1H), 7.26 (d, J=7.6 Hz, 1H), 7.05-6.99 (m, 1H), 6.97-6.91 (m, 1H), 4.79-4.70 (m, 1H), 4.50-4.40 (m, 1H), 3.28-3.20 (m, 1H), 3.13-2.84 (m, 3H), 2.40-2.30 (m, 1H), 2.30-2.20 (m, 1H), 1.57 (d, J=6.4 Hz, 3H); ES-LCMS m/z 426.2 [M−NH₂]⁺.

### Example 34

### Synthesis of Compound I-33

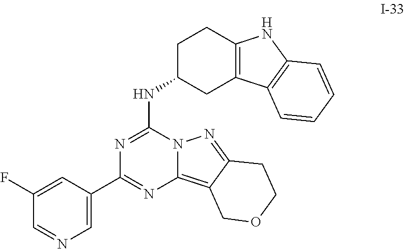 [SMILES: Fc1cncc(-c2nc(N[C@@H]3CCc4[nH]c5ccccc5c4C3)n3nc4c(c3n2)COCC4)c1]

### Synthetic Scheme:

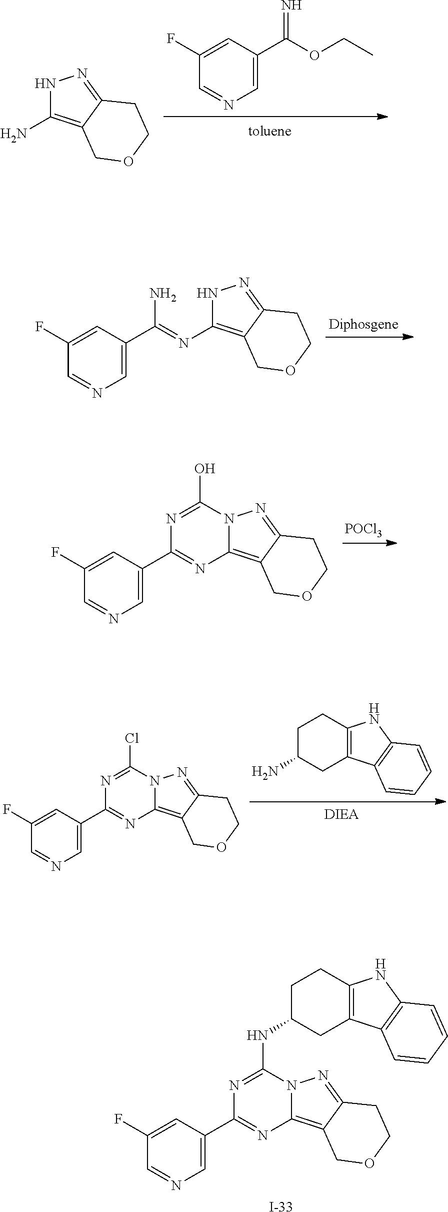

### Step 1: 5-Fluoro-N′-(2,4,6,7-tetrahydropyrano[4,3-c]pyrazol-3-yl)pyridine-3-carboxamidine

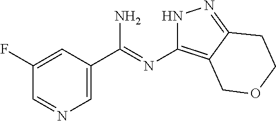 [SMILES: N/C(=N\c1[nH]nc2c1COCC2)c1cncc(F)c1]

To a stirred solution of ethyl 5-fluoropyridine-3-carboximidate (381.63 mg, 2.16 mmol, 1 eq) in toluene (8 mL) was added 2,4,6,7-tetrahydropyrano[4,3-c]pyrazol-3-amine (300 mg, 2.16 mmol, 1 eq). The reaction mixture was stirred at 120° C. for 12 h under N₂ atmosphere. The reaction mixture was cooled to 0° C. under ice-water bath and filtered. The filtered cake was concentrated to yield 5-fluoro-N-(2,4,6,7-tetrahydropyrano[4,3-c]pyrazol-3-yl)pyridine-3-carboxamidine (415 mg, 1.54 mmol, 71.3% yield, 96.8% purity) as a yellow solid which was used in the next step without further purification. ¹H NMR (400 MHz, CD3OD) δ ppm 8.89 (s, 1H), 8.58 (d, J=2.7 Hz, 1H), 8.11-8.04 (m, 1H), 4.71 (s, 2H), 3.93 (t, J=5.5 Hz, 2H), 2.75 (t, J=5.5 Hz, 2H); ES-LCMS m/z 262.1 [M+H]⁺.

### Step 2: 2-(5-Fluoro-3-pyridyl)-8,10-dihydro-7H-pyrano[2,3]pyrazolo[2,4-c][1,3,5]triazin-4-ol

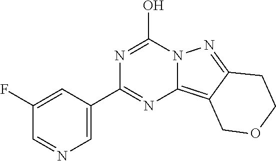 [SMILES: Oc1nc(-c2cncc(F)c2)nc2c3c(nn12)CCOC3]

To a stirred solution of 5-fluoro-N-(2,4,6,7-tetrahydropyrano[4,3-c]pyrazol-3-yl)pyridine-3-carboxamidine (415 mg, 1.54 mmol, 1 eq) in 1,4-dioxane (2.5 mL) and THE (2.5 mL) was added diphosgene (760.49 mg, 3.84 mmol, 463.71 μL, 2.5 eq). The reaction mixture was stirred at 80° C. for 12 h. The mixture was cooled to 28° C. and filtered. The filtered cake was concentrated to yield 2-(5-fluoro-3-pyridyl)-8,10-dihydro-7H-pyrano[2,3]pyrazolo[2,4-c][1,3,5]triazin-4-ol (450 mg, crude, HCl) as a brown solid which was used in the next step without further purification. ¹H NMR (400 MHz, CD3OD) δ ppm 9.16 (s, 1H), 8.82 (d, J=2.7 Hz, 1H), 8.46 (td, J=2.3, 8.8 Hz, 1H), 4.87 (s, 2H), 4.03 (t, J=5.7 Hz, 2H), 2.92 (t, J=5.6 Hz, 2H); ES-LCMS m/z 288.1 [M+H]⁺.

### Step 3: 4-Chloro-2-(5-fluoro-3-pyridyl)-8,10-dihydro-7H-pyrano[2,3]pyrazolo[2,4-c][1,3,5]triazine

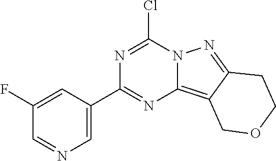 [SMILES: Fc1cncc(-c2nc(Cl)n3nc4c(c3n2)COCC4)c1]

To a stirred solution of 2-(5-fluoro-3-pyridyl)-8,10-dihydro-7H-pyrano[2,3]pyrazolo [2,4-c][1,3,5]triazin-4-ol (150 mg, 463.38 μmol, 1 eq, HCl) in toluene (5 mL) was added DIEA (179.66 mg, 1.39 mmol, 242.13 μL, 3 eq) and POCl₃ (726.84 mg, 4.74 mmol, 440.51 μL, 10.23 eq). The reaction mixture was stirred at 120° C. for 2 h. The reaction mixture was concentrated to yield 4-chloro-2-(5-fluoro-3-pyridyl)-8,10-dihydro-7H-pyrano[2,3]pyrazolo[2,4-c][1,3,5]triazine (160 mg, crude, 2HCl) as a brown solid which was used in the next step without further purification. ES-LCMS m/z 306.1, 308.0 [M+H]⁺.

### Step 4: (3R)—N-[8-Isopropyl-2-(1,2,4-triazol-1-yl)pyrazolo[1,5-a][1,3,5]triazin-4-yl]-2,3,4,9-tetrahydro-1H-carbazol-3-amine (I-33)

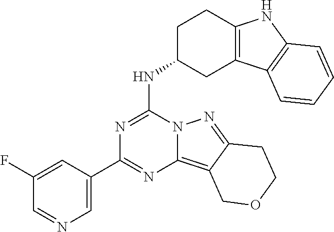 [SMILES: Fc1cncc(-c2nc(N[C@@H]3CCc4[nH]c5ccccc5c4C3)n3nc4c(c3n2)COCC4)c1]

To a solution of 4-chloro-2-(5-fluoro-3-pyridyl)-8,10-dihydro-7H-pyrano[2,3]pyrazolo[2,4-c][1,3,5]triazine (160 mg, 422.59 μmol, 1 eq, 2HCl) in ACN (10 mL) was added DIEA (273.08 mg, 2.11 mmol, 368.03 μL, 5 eq) and (3R)-2,3,4,9-tetrahydro-1H-carbazol-3-amine (82.64 mg, 443.72 μmol, 1.05 eq). The mixture was stirred at 60° C. for 1 h. The reaction mixture was concentrated to yield a residue which was purified by preparative HPLC (HCl condition; column: DuraShell 150*25 mm*5 um; mobile phase: [water (0.04% HCl)−ACN]; B %: 52%-82%, 8 min). The desired fraction was lyophilized to yield 2-(5-fluoro-3-pyridyl)-N-[(3R)-2,3,4,9-tetrahydro-1H-carbazol-3-yl]-8,10-dihydro-7H-pyrano[2,3]pyrazolo[2,4-c][1,3,5]triazin-4-amine (34.1 mg, 64.53 μmol, 15.3% yield, 100.0% purity, 2HCl, [α]^{28.6}_{D}=+17.400 (DMSO, c=0.050 g/100 mL) as a yellow solid. ¹H NMR (400 MHz, CD3OD) δ ppm 9.41 (s, 1H), 8.80-8.71 (m, 2H), 7.36 (d, J=7.8 Hz, 1H), 7.27 (d, J=8.0 Hz, 1H), 7.03 (t, J=7.0 Hz, 1H), 6.98-6.91 (m, 1H), 4.91 (s, 2H), 4.86-4.85 (m, 1H), 4.04 (t, J=5.6 Hz, 2H), 3.29-3.24 (m, 1H), 3.08-2.87 (m, 5H), 2.36 (d, J=6.3 Hz, 1H), 2.32-2.19 (m, 1H); ES-LCMS m/z 456.2 [M+H]⁺.

### Example 35

### Synthesis of Compound I-34a

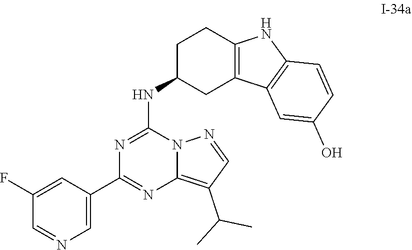 [SMILES: CC(C)c1cnn2c(N[C@H]3CCc4[nH]c5ccc(O)cc5c4C3)nc(-c3cncc(F)c3)nc12]

### Synthetic Scheme:

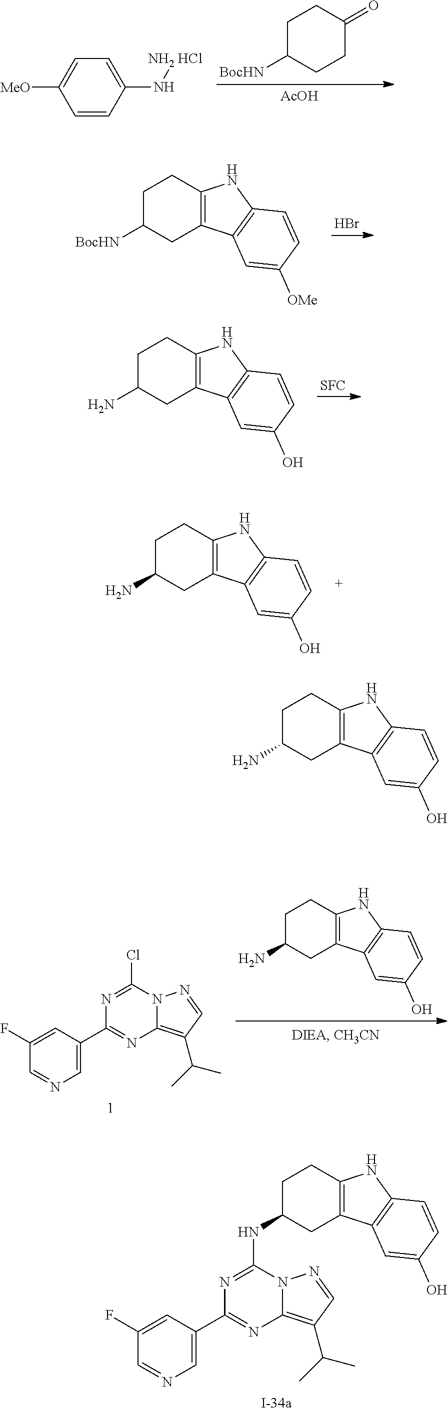

### Step 1: tert-Butyl N-(6-methoxy-2,3,4,9-tetrahydro-1H-carbazol-3-yl)carbamate

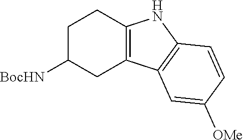 [SMILES: COc1ccc2[nH]c3c(c2c1)CC(NC(=O)OC(C)(C)C)CC3]

To a solution of (4-methoxyphenyl)hydrazine (2 g, 11.45 mmol, 1 eq, HCl) in EtOH (40 mL) was added tert-butyl N-(4-oxocyclohexyl)carbamate (2.44 g, 11.45 mmol, 2.44 mL, 1 eq) and then the mixture was stirred at 70° C. for 1 h. The mixture was concentrated to yield a residue which was purified on silica gel column chromatography (from PE/EtOAc=100/1 to 3/1, TLC: PE/EtOAc=3/1, R_{f}=0.4) to yield tert-butyl N-(6-methoxy-2,3,4,9-tetrahydro-1H-carbazol-3-yl)carbamate (2.9 g, 8.71 mmol, 76.1% yield, 95.0% purity) as a yellow solid. ¹H NMR (400 MHz, CD3OD) δ ppm 7.11 (d, J=8.8 Hz, 1H), 6.85 (d, J=2.4 Hz, 1H), 6.66 (dd, J=2.4, 8.8 Hz, 1H), 3.88-3.81 (m, 1H), 3.79 (s, 3H), 2.97 (dd, J=5.3, 14.8 Hz, 1H), 2.86-2.75 (m, 2H), 2.48 (dd, J=9.2, 14.8 Hz, 1H), 2.11 (dd, J=3.3, 8.2 Hz, 1H), 1.88-1.75 (m, 1H), 1.46 (s, 9H); ES-LCMS m z 317.2 [M+H]⁺.

### Step 2: (6S)-6-Amino-6,7,8,9-tetrahydro-5H-carbazol-3-ol & (6R)-6-Amino-6,7,8,9-tetrahydro-5H-carbazol-3-ol

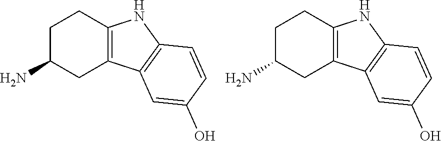

A solution of tert-butyl N-(6-methoxy-2,3,4,9-tetrahydro-1H-carbazol-3-yl)carbamate (500 mg, 1.50 mmol, 1 eq) in HBr (10 mL) was stirred at 120° C. for 2 h. TLC (PE/EtOAc=3/1, R_{f}=0.40) indicated starting material was disappeared, and one major new spot with larger polarity was detected. The reaction mixture was concentrated to yield a residue which was separated by chiral SFC (column: DAICEL CHIRALPAK AD (250 mm*30 mm, 10 um); mobile phase: [0.1% NH₃.H₂OEtOH]; B %: 50%-50%) to yield (6S)-6-amino-6,7,8,9-tetrahydro-5H-carbazol-3-ol (90 mg, 432.98 μmol, 28.9% yield, 97.3% purity, SFC: Rₜ=3.057) as a gray solid. ¹H NMR (400 MHz, CD3OD) δ ppm 7.04 (d, J=8.6 Hz, 1H), 6.73 (d, J=2.2 Hz, 1H), 6.56 (dd, J=2.4, 8.6 Hz, 1H), 3.23-3.11 (m, 1H), 2.92 (dd, J=4.6, 14.7 Hz, 1H), 2.85-2.75 (m, 2H), 2.37 (dd, J=8.8, 14.7 Hz, 1H), 2.11-2.02 (m, 1H), 1.75 (dd, J=7.9, 10.2, 12.6 Hz, 1H); ES-LCMS m/z 203.2 [M+H]⁺. And (6R)-6-amino-6,7,8,9-tetrahydro-5H-carbazol-3-ol (90 mg, 435.20 μmol, 29.0% yield, 97.8% purity, SFC: Rₜ=3.863) as a gray solid. ¹H NMR (400 MHz, CD3OD) δ ppm 7.04 (d, J=8.6 Hz, 1H), 6.73 (d, J=2.2 Hz, 1H), 6.56 (dd, J=2.2, 8.6 Hz, 1H), 3.23-3.12 (m, 1H), 2.92 (dd, J=5.0, 15.0 Hz, 1H), 2.84-2.76 (m, 2H), 2.38 (dd, J=8.8, 14.9 Hz, 1H), 2.07 (td, J=4.2, 8.2 Hz, 1H), 1.83-1.69 (m, 1H); ES-LCMS m/z 203.2 [M+H]⁺.

### Step 3: (6S)-6-[[2-(5-Fluoro-3-pyridyl)-8-isopropyl-pyrazolo[1,5-a][1,3,5]triazin-4-yl]amino]-6,7,8,9-tetrahydro-5H-carbazol-3-ol (I-34a)

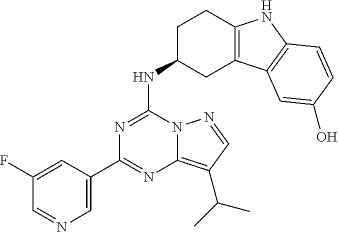 [SMILES: CC(C)c1cnn2c(N[C@H]3CCc4[nH]c5ccc(O)cc5c4C3)nc(-c3cncc(F)c3)nc12]

A mixture of 4-chloro-2-(5-fluoro-3-pyridyl)-8-isopropyl-pyrazolo[1,5-a][1,3,5]triazine (100 mg, 336.29 μmol, 1 eq), (6S)-6-amino-6,7,8,9-tetrahydro-5H-carbazol-3-ol (69.90 mg, 336.29 μmol, 1 eq) and DIEA (173.85 mg, 1.35 mmol, 234.30 μL, 4 eq) in ACN (10 mL) was stirred at 80° C. for 10 h. The reaction mixture was concentrated to yield a residue which was purified by preparative HPLC (column: Agela ASB 150*25 mm*5 um; mobile phase: [water (0.05% HCl)−ACN]; B %: 55%-85%, 8 min), followed by lyophilization to yield (6S)-6-[[2-(5-fluoro-3-pyridyl)-8-isopropyl-pyrazolo[1,5-a][1,3,5]triazin-4-yl]amino]-6,7,8,9-tetrahydro-5H-carbazol-3-ol (16.09 mg, 28.70 μmol, 8.5% yield, 94.6% purity, 2HCl, SFC: Rₜ=2.082, ee=97.2%, [α]^{31.0}_{D}=−54.724, DMSO, c=0.040 g/100 mL) as a yellow solid. ¹H NMR (400 MHz, DMSO-d₆) δ ppm 10.45 (s, 1H), 9.38 (s, 1H), 8.98 (d, J=8.5 Hz, 1H), 8.70 (d, J=2.8 Hz, 1H), 8.44 (dd, J=1.5, 9.8 Hz, 1H), 8.15 (s, 1H), 7.07 (d, J=8.5 Hz, 1H), 6.67 (d, J=2.3 Hz, 1H), 6.53 (dd, J=2.3, 8.5 Hz, 1H), 4.80 (m, 1H), 3.23 (td, J=6.8, 13.7 Hz, 1H), 3.04-2.95 (m, 2H), 2.92-2.81 (m, 2H), 2.19 (d, J=3.3 Hz, 2H), 1.38 (d, J=7.0 Hz, 6H); ES-LCMS m/z 458.2 [M+H]⁺.

### Example 36

### Synthesis of Compound I-34b

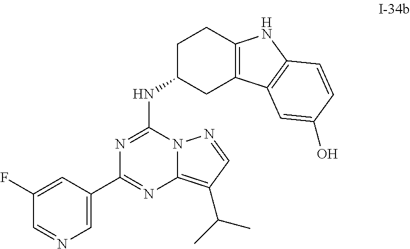 [SMILES: CC(C)c1cnn2c(N[C@@H]3CCc4[nH]c5ccc(O)cc5c4C3)nc(-c3cncc(F)c3)nc12]

### Synthetic Scheme:

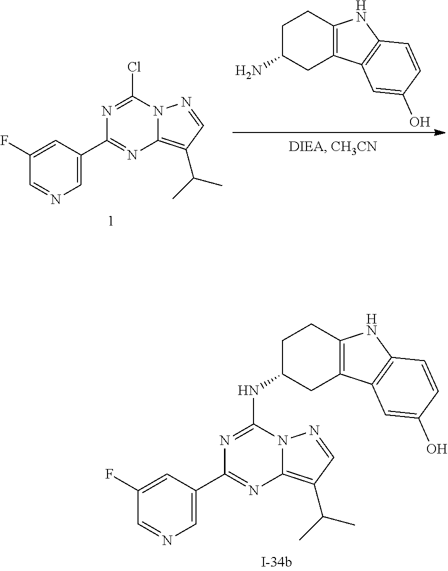 [SMILES: CC(C)c1cnn2c(Cl)nc(-c3cncc(F)c3)nc12 ; N[C@@H]1CCc2[nH]c3ccc(O)cc3c2C1 ; CC(C)c1cnn2c(N[C@@H]3CCc4[nH]c5ccc(O)cc5c4C3)nc(-c3cncc(F)c3)nc12]

### Step 1: (6R)-6-[[2-(5-Fluoro-3-pyridyl)-8-isopropyl-pyrazolo[1,5-a][1,3,5]triazin-4-yl]amino]-6,7,8,9-tetrahydro-5H-carbazol-3-ol (I-34b)

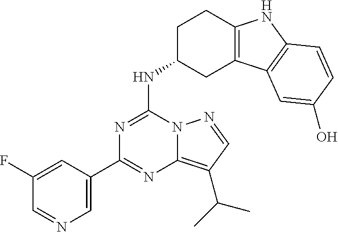 [SMILES: CC(C)c1cnn2c(N[C@@H]3CCc4[nH]c5ccc(O)cc5c4C3)nc(-c3cncc(F)c3)nc12]

A mixture of 4-chloro-2-(5-fluoro-3-pyridyl)-8-isopropyl-pyrazolo[1,5-a][1,3,5]triazine (100 mg, 336.29 μmol, 1 eq), (6R)-6-amino-6,7,8,9-tetrahydro-5H-carbazol-3-ol (69.55 mg, 336.29 μmol, 1 eq) and DIEA (173.85 mg, 1.35 mmol, 234.30 μL, 4 eq) in ACN (10 mL) was stirred at 80° C. for 10 h. The reaction mixture was concentrated. The residue was purified by preparative HPLC (column: Agela ASB 150*25 mm*5 um; mobile phase: [water (0.05% HCl)−ACN]; B %: 55%-85%, 8 min) followed by lyophilization to yield (6R)-6-[[2-(5-fluoro-3-pyridyl)-8-isopropyl-pyrazolo[1,5-a][1,3,5]triazin-4-yl]amino]-6,7,8,9-tetrahydro-5H-carbazol-3-ol (15.02 mg, 27.13 μmol, 8.1% yield, 95.8% purity, 2HCl, SFC: Rₜ=3.105, [α]^{30.9}_{D}=+14.611, DMSO, c=0.022 g/100 mL) as a yellow solid. ¹H NMR (400 MHz, DMSO-d₆) δ ppm 10.45 (s, 1H), 9.38 (s, 1H), 8.98 (d, J=8.5 Hz, 1H), 8.71 (d, J=2.8 Hz, 1H), 8.44 (dd, J=1.6, 9.9 Hz, 1H), 8.15 (s, 1H), 7.09-7.04 (m, 1H), 6.66 (d, J=2.3 Hz, 1H), 6.53 (dd, J=2.3, 8.5 Hz, 1H), 4.80 (m, 1H), 3.24 (td, J=7.0, 13.9 Hz, 1H), 3.04-2.95 (m, 2H), 2.91-2.81 (m, 2H), 2.18 (s, 2H), 1.38 (d, J=6.8 Hz, 6H); ES-LCMS m/z 458.2 [M+H]⁺.

### Example 37

### Synthesis of Compound I-35a

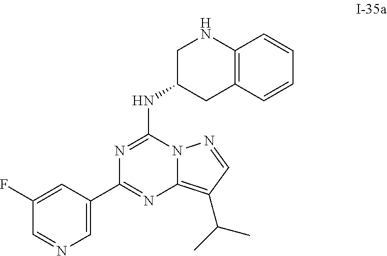 [SMILES: CC(C)c1cnn2c(N[C@@H]3CNc4ccccc4C3)nc(-c3cncc(F)c3)nc12]

### Synthetic Scheme:

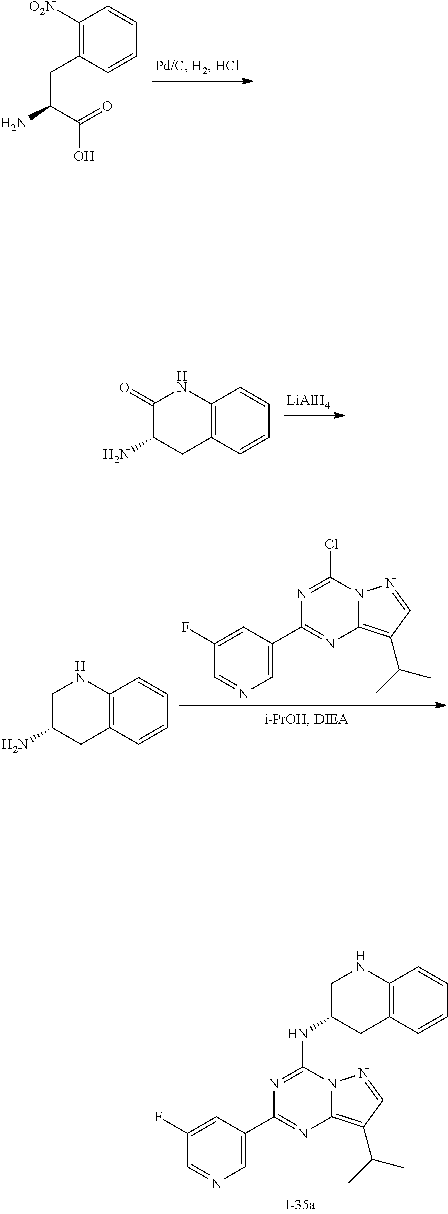

### Step 1: (3S)-3-Amino-3,4-dihydro-1H-quinolin-2-one

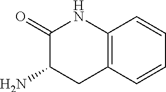 [SMILES: N[C@H]1Cc2ccccc2NC1=O]

To a solution of (2S)-2-amino-3-(2-nitrophenyl)propanoic acid (500 mg, 2.38 mmol, 1 eq) in MeOH (20 mL) was added HCl (12 M, 198.24 μL, 1 eq) and Pd/C (1 g, 10% purity). The reaction mixture was stirred at 40° C. under H₂ atmosphere (50 psi) for 16 h. The reaction mixture was filtered and concentrated to yield (3S)-3-amino-3,4-dihydro-1H-quinolin-2-one (420 mg, 2.33 mmol, 98.0% yield, 90.0% purity) as a yellow solid which was used in the next step without purification. ¹H NMR (400 MHz, CD3OD) δ ppm 7.29-7.23 (m, 2H), 7.09-7.03 (m, 1H), 6.93 (d, J=7.8 Hz, 1H), 4.20 (dd, J=6.7, 14.7 Hz, 1H), 3.29-3.23 (m, 1H), 3.19-3.10 (m, 1H); ES-LCMS m/z 163.3 [M+H]⁺.

### Step 2: (3S)-1,2,3,4-Tetrahydroquinolin-3-amine

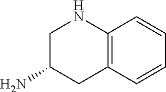 [SMILES: N[C@@H]1CNc2ccccc2C1]

To a solution of (3S)-3-amino-3,4-dihydro-1H-quinolin-2-one (120 mg, 665.89 μmol, 1 eq) in THF (15 mL) was added LAH (126.35 mg, 3.33 mmol, 5 eq) at 0° C. in portions. The reaction mixture was stirred at 40° C. for 2 h. H₂O (1 mL), 10% NaOH solution (1 mL) and H₂O (3 mL) were added slowly to quench the reaction. Then MgSO₄ was added and filtered. The filtrate was concentrated to yield (3S)-1,2,3,4-tetrahydroquinolin-3-amine (120 mg, 603.22 μmol, 90.6% yield, 74.5% purity) as a yellow solid which was used in the next step without purification. ¹H NMR (400 MHz, CD3OD) δ ppm 6.95-6.88 (m, 2H), 6.60-6.50 (m, 2H), 3.32-3.30 (m, 1H), 3.20 (ddt, J=3.5, 4.7, 7.8 Hz, 1H), 3.01-2.93 (m, 2H), 2.57 (dd, J=7.5, 15.8 Hz, 1H); ES-LCMS m z 149.1 [M+H]⁺.

### Step 3: (3S)—N-[2-(5-Fluoro-3-pyridyl)-8-isopropyl-pyrazolo[1,5-a][1,3,5]triazin-4-yl]-1,2,3,4-tetrahydroquinolin-3-amine (I-35a)

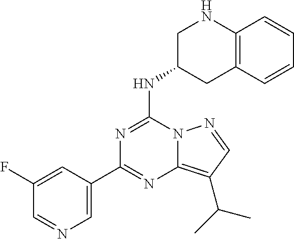 [SMILES: CC(C)c1cnn2c(N[C@@H]3CNc4ccccc4C3)nc(-c3cncc(F)c3)nc12]

A mixture of 4-chloro-2-(5-fluoro-3-pyridyl)-8-isopropyl-pyrazolo[1,5-a][1,3,5]triazine (100 mg, 336.29 μmol, 1 eq), (3S)-1,2,3,4-tetrahydroquinolin-3-amine (66.90 mg, 336.29 μmol, 1 eq) and DIEA (173.85 mg, 1.35 mmol, 234.30 μL, 4 eq) in ACN (10 mL) was stirred at 80° C. for 2 h. The reaction mixture was concentrated to a residue which was purified by preparative HPLC (column: Agela ASB 150*25 mm*5 um; mobile phase: [water (0.05% HCl)−ACN]; B %: 65%-95%, 8 min), followed by lyophilization to yield (3S)—N-[2-(5-fluoro-3-pyridyl)-8-isopropyl-pyrazolo[1,5-a][1,3,5]triazin-4-yl]-1,2,3,4-tetrahydroquinolin-3-amine (69.33 mg, 132.86 μmol, 39.5% yield, 98.3% purity, 3HCl, SFC: Rₜ=4.759, ee=99%, [α]^{29.2}_{D}=5.265, DMSO, c=0.032 g/100 mL) as a yellow solid. ¹H NMR (400 MHz, CD3OD) δ ppm 9.47 (s, 1H), 8.67-8.59 (m, 2H), 8.02 (s, 1H), 7.33-7.26 (m, 2H), 7.21-7.13 (m, 1H), 7.10 (d, J=8.0 Hz, 1H), 5.06 (s, 1H), 3.83 (d, J=11.3 Hz, 1H), 3.68-3.59 (m, 1H), 3.47-3.38 (m, 1H), 3.32-3.21 (m, 2H), 1.43 (d, J=7.0 Hz, 6H); ES-LCMS m/z 404.2 [M+H]⁺.

### Example 38

### Synthesis of Compound I-35b

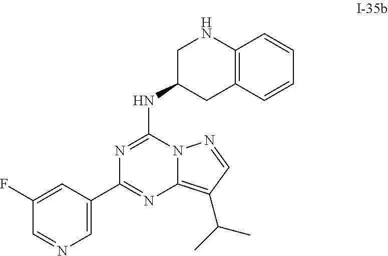 [SMILES: CC(C)c1cnn2c(N[C@H]3CNc4ccccc4C3)nc(-c3cncc(F)c3)nc12]

### Synthetic Scheme:

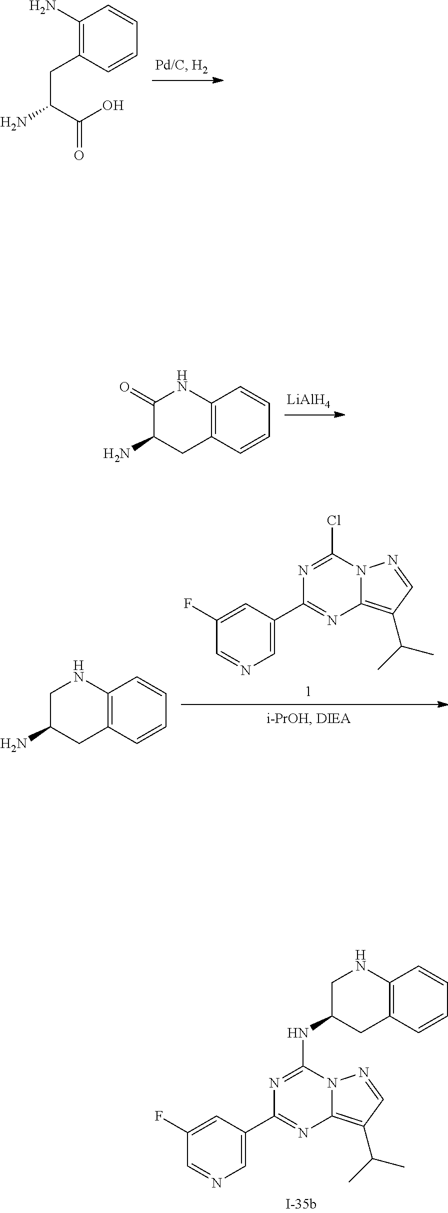

### Step 1: (3R)-3-Amino-3,4-dihydro-1H-quinolin-2-one

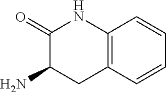 [SMILES: N[C@@H]1Cc2ccccc2NC1=O]

To a solution of (2R)-2-amino-3-(2-nitrophenyl)propanoic acid (500.00 mg, 2.38 mmol, 1 eq) in MeOH (20 mL) and con HCl (12 M, 0.1 mL, 36% purity, 0.5 eq) was added Pd/C (50 mg, 2.38 mmol, 10% purity) under N₂ atmosphere. The mixture was stirred for 12 h at 40° C. under H₂ atmosphere. The mixture was filtered and the filtrate was concentrated to yield (3R)-3-amino-3,4-dihydro-1H-quinolin-2-one (500 mg, 2.27 mmol, 95.2% yield, 90.0% purity, HCl) as an off white solid which was used in the next step without purification. ¹H NMR (400 MHz, CD3OD) δ ppm 7.31-7.24 (m, 2H), 7.11-7.03 (m, 1H), 6.95 (d, J=7.8 Hz, 1H), 4.22 (dd, J=6.7, 14.7 Hz, 1H), 3.31-3.25 (m, 1H), 3.21-3.12 (m, 1H); ES-LCMS m/z 163.3 [M+H]⁺.

### Step 2: (R)-1,2,3,4-Tetrahydroquinolin-3-amine

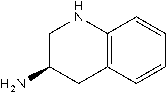 [SMILES: N[C@H]1CNc2ccccc2C1]

To a solution of (3R)-3-amino-3,4-dihydro-1H-quinolin-2-one (150 mg, 832.36 μmol, 1 eq, HCl) in THE (20 mL) was added LAH (157.94 mg, 4.16 mmol, 5 eq) under N₂ atmosphere at 0° C. The mixture was stirred at 40° C. for 3 h. H₂O (0.5 mL), 10% NaOH solution (0.5 mL) and H₂O (1.5 mL) were added slowly in sequence to quench the reaction. Then Na₂SO₄ was added and filtered. The filtrate was concentrated to yield (3R)-1,2,3,4-tetrahydroquinolin-3-amine (100 mg, crude) as brown oil which was used in the next step without purification. ¹H NMR (400 MHz, CD3OD) δ ppm 6.93-6.86 (m, 2H), 6.57-6.48 (m, 2H), 3.29-3.26 (m, 1H), 3.17 (s, 1H), 2.99-2.91 (m, 2H), 2.55 (dd, J=7.6, 16.4 Hz, 1H); ES-LCMS m/z 149.1 [M+H]⁺.

### Step 3: (R)—N-(2-(5-Fluoropyridin-3-yl)-8-isopropylpyrazolo[1,5-a][1,3,5]triazin-4-yl)-1,2,3,4-tetrahydroquinolin-3-amine (I-35b)

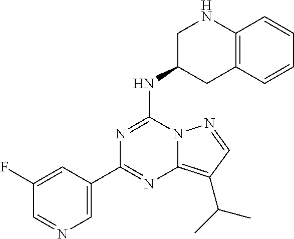 [SMILES: CC(C)c1cnn2c(N[C@H]3CNc4ccccc4C3)nc(-c3cncc(F)c3)nc12]

To a solution of (3R)-1,2,3,4-tetrahydroquinolin-3-amine (100 mg, 674.74 μmol, 1 eq) in CH₃CN (15 mL) was added 4-chloro-2-(5-fluoro-3-pyridyl)-8-isopropyl-pyrazolo[1,5-a][1,3,5]triazine (200.64 mg, 674.74 μmol, 1 eq) and DIEA (261.62 mg, 2.02 mmol, 352.58 μL, 3 eq). The mixture was stirred at 60° C. for 2 h. The mixture was concentrated to yield a residue which was separated by SFC (column: DAICEL CHIRALPAK AD-H (250 mm*30 mm, 5 μm); mobile phase: [0.1% NH₃.H₂OEtOH]; B %: 35%-35%, 8 min). The desired fraction was concentrated to yield (3R)—N-[2-(5-fluoro-3-pyridyl)-8-isopropyl-pyrazolo[1,5-a][1,3,5]triazin-4-yl]-1,2,3,4-tetrahydroquinolin-3-amine (21.48 mg, 53.24 μmol, 7.8% yield, 100% purity, SFC: Rt=5.123 min, ee=99.58%, [α]30.2_{D}=−37.52 (MeOH, c=0.045 g/100 mL)) as a yellow solid. ¹H NMR (400 MHz, CD3OD) δ ppm 9.43 (s, 1H), 8.60-8.49 (m, 2H), 7.94 (s, 1H), 7.03-6.92 (m, 2H), 6.66-6.58 (m, 2H), 4.94 (m, 1H), 3.59 (d, J=11.5 Hz, 1H), 3.41 (dd, J=6.6, 11.2 Hz, 1H), 3.30-3.20 (m, 2H), 3.14-3.01 (m, 1H), 1.40 (d, J=6.8 Hz, 6H); ES-LCMS m/z 404.2 [M+H]⁺.

### Example 39

### Synthesis of Compound I-36a and I-36b

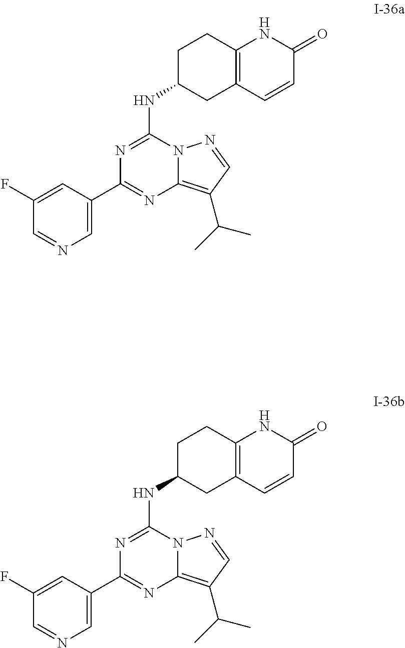 [SMILES: CC(C)c1cnn2c(N[C@@H]3CCc4[nH]c(=O)ccc4C3)nc(-c3cncc(F)c3)nc12 ; CC(C)c1cnn2c(N[C@H]3CCc4[nH]c(=O)ccc4C3)nc(-c3cncc(F)c3)nc12]

### Synthetic Scheme:

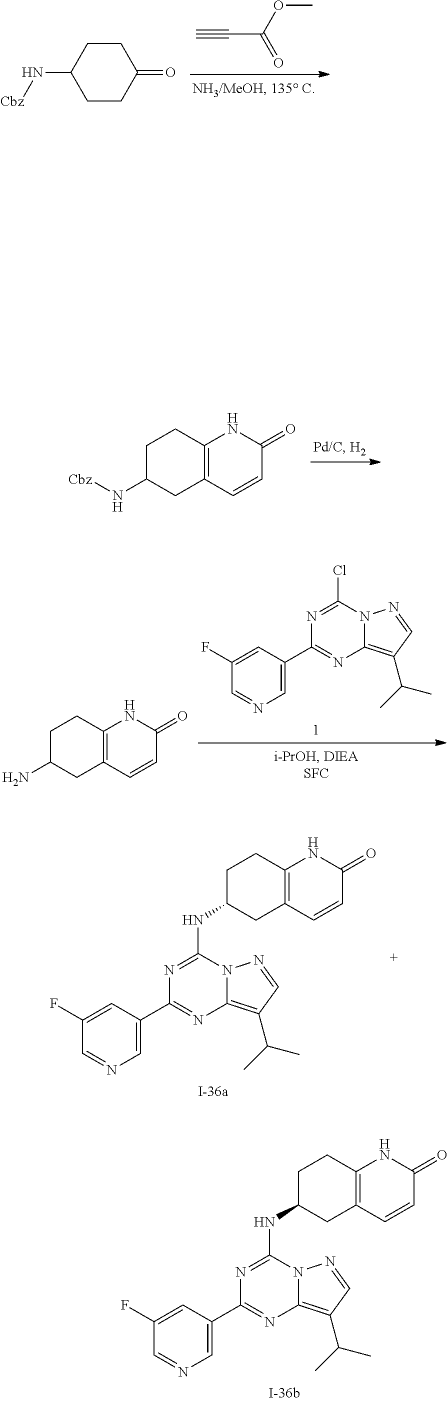

### Step 1: Benzyl N-(2-oxo-5,6,7,8-tetrahydro-1H-quinolin-6-yl)carbamate

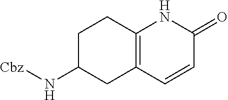 [SMILES: O=C(NC1CCc2[nH]c(=O)ccc2C1)OCc1ccccc1]

A mixture of benzyl N-(4-oxocyclohexyl)carbamate (2 g, 8.09 mmol, 1 eq), methyl prop-2-ynoate (1.43 g, 16.98 mmol, 1.41 mL, 2.1 eq), NH₃/MeOH (7 M, 20 mL, 17.31 eq) in i-PrOH (15 mL) was stirred at 135° C. for 12 h. The reaction mixture was concentrated under reduced pressure to yield a residue which was added MeOH (10 mL), stirred for 0.5 h. The slurry was filtered, rinsed with MeOH (5 mL×2), collected and dried in vacuo to yield benzyl N-(2-oxo-5,6,7,8-tetrahydro-1H-quinolin-6-yl)carbamate (900 mg, 3.02 mmol, 37.3% yield, 100% purity) as a light yellow solid. ¹H NMR (400 MHz, DMSO-d₆) δ ppm 7.41-7.31 (m, 5H), 7.13 (d, J=9.3 Hz, 1H), 6.10 (d, J=9.0 Hz, 1H), 5.02 (s, 2H), 3.65 (m, 1H), 2.68-2.52 (m, 3H), 2.30 (dd, J=8.8, 15.7 Hz, 1H), 1.88 (dd, J=4.2, 8.3 Hz, 1H), 1.68-1.49 (m, 1H); ES-LCMS m/z 299.2 [M+H]⁺.

### Step 2: 6-Amino-5,6,7,8-tetrahydro-1H-quinolin-2-one

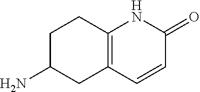 [SMILES: NC1CCc2[nH]c(=O)ccc2C1]

To a solution of benzyl N-(2-oxo-5,6,7,8-tetrahydro-1H-quinolin-6-yl)carbamate (300 mg, 1.01 mmol, 1 eq) in THE (10 mL) and MeOH (10 mL) was added Pd/C (80 mg, 10%) under N₂ atmosphere. The mixture was stirred at 25° C. for 12 h under H₂ (50 psi). The mixture was filtered, the cake was rinsed with MeOH (30 mL×2). The filtrate was concentrated under reduced pressure to yield 6-amino-5,6,7,8-tetrahydro-1H-quinolin-2-one (165 mg, 897.33 μmol, 89.2% yield, 89.3% purity) as a white solid which was used in the next step without further purification. ¹H NMR (400 MHz, CD3OD) δ ppm 7.34 (d, J=9.3 Hz, 1H), 6.34 (d, J=9.0 Hz, 1H), 3.09 (m, 1H), 2.80-2.67 (m, 3H), 2.32 (dd, J=9.2, 15.7 Hz, 1H), 2.01 (br s, 1H), 1.73-1.53 (m, 1H); ES-LCMS m/z 165.2 [M+H]⁺.

### Step 3: (R)-6-((2-(5-Fluoropyridin-3-yl)-8-isopropylpyrazolo[1,5-a][1,3,5]triazin-4-yl)amino)-5,6,7,8-tetrahydroquinolin-2(1H)-one (I-36a) & (S)-6-((2-(5-Fluoropyridin-3-yl)-8-isopropylpyrazolo[1,5-a][1,3,5]triazin-4-yl)amino)-5,6,7,8-tetrahydroquinolin-2(1H)-one (I-36b)

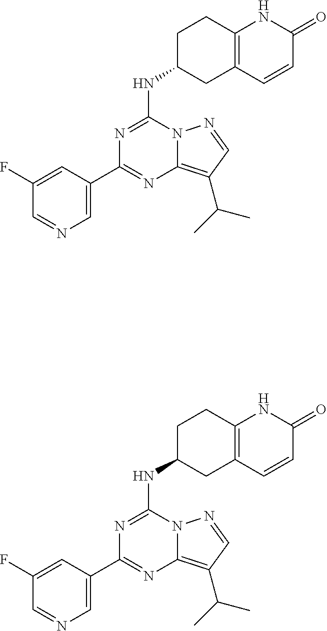 [SMILES: CC(C)c1cnn2c(N[C@@H]3CCc4[nH]c(=O)ccc4C3)nc(-c3cncc(F)c3)nc12 ; CC(C)c1cnn2c(N[C@H]3CCc4[nH]c(=O)ccc4C3)nc(-c3cncc(F)c3)nc12]

To a solution of 4-chloro-2-(5-fluoro-3-pyridyl)-8-isopropyl-pyrazolo[1,5-a][1,3,5]triazine (266.83 mg, 897.33 μmol, 1 eq) in i-PrOH (10 mL) was added DIEA (347.91 mg, 2.69 mmol, 468.88 μL, 3 eq) and 6-amino-5,6,7,8-tetrahydro-1H-quinolin-2-one (165 mg, 897.33 μmol, 1 eq). The mixture was stirred at 60° C. for 12 h. The reaction mixture was concentrated to yield a residue which was purified by flash silica gel chromatography (from EtOAc to EtOAc/MeOH=10/1, TLC: DCM/MeOH=10/1, R_{f}=0.54) to yield the crude product which was separated by SFC (column: DAICEL CHIRALPAK AD-H (250 mm*30 mm, 5 μm); mobile phase: [0.1% NH₃—H₂OIPA]; B %: 40%-40%). The desired fractions were concentrated to yield two crude products. One of the crude products was purified by preparative HPLC (HCl condition; column: DuraShell 150*25 mm*5 μm; mobile phase: [water (0.05% HCl)−ACN]; B %: 57%-87%, 8 min), followed by lyophilization to yield an enantiomer (64.26 mg, 126.33 μmol, 21.5% yield, 96.8% purity, 2 HCl, SFC: Rₜ=1.870 min, ee=96.48%, [α]^{29.7}_{D}=+11.184 (MeOH, c=0.048 g/100 mL)) as a yellow solid. ¹H NMR (400 MHz, DMSO-d₆) δ ppm 12.42-11.51 (m, 1H), 9.41-9.38 (m, 1H), 9.01 (d, J=8.5 Hz, 1H), 8.71 (d, J=2.8 Hz, 1H), 8.50-8.44 (m, 1H), 8.14 (s, 1H), 7.32 (d, J=9.3 Hz, 1H), 6.28 (d, J=9.3 Hz, 1H), 4.71 (m, 1H), 3.27-3.15 (m, 1H), 2.91-2.68 (m, 4H), 2.15-1.90 (m, 2H), 1.36 (d, J=7.0 Hz, 6H); ES-LCMS m/z 420.2 [M+H]⁺. The other of these crude products was purified by preparative HPLC (HCl condition; column: DuraShell 150*25 mm*5 μm; mobile phase: [water (0.05% HCl)−ACN]; B %: 57%-87%, 8 min), followed by lyophilization to yield the other enantiomer (38.10 mg, 77.38 μmol, 13.2% yield, 100.0% purity, 2 HCl, SFC: Rt=2.055 min, ee=95.44%, [α]^{29.8}_{D}=−13.070 (MeOH, c=0.044 g/100 mL)) as a yellow solid. ¹H NMR (400 MHz, DMSO-d₆) δ ppm 11.72 (s, 1H), 9.44-9.37 (m, 1H), 9.00 (d, J=8.8 Hz, 1H), 8.71 (d, J=3.0 Hz, 1H), 8.51-8.43 (m, 1H), 8.14 (s, 1H), 7.27 (d, J=9.3 Hz, 1H), 6.22 (d, J=9.3 Hz, 1H), 4.71 (m, 1H), 3.21 (td, J=7.0, 13.9 Hz, 1H), 2.92-2.66 (m, 4H), 2.13-1.90 (m, 2H), 1.36 (d, J=7.0 Hz, 6H); ES-LCMS m/z 420.2 [M+H]⁺.

### Example 40

### Synthesis of Compound I-37a, I-37b and I-37c

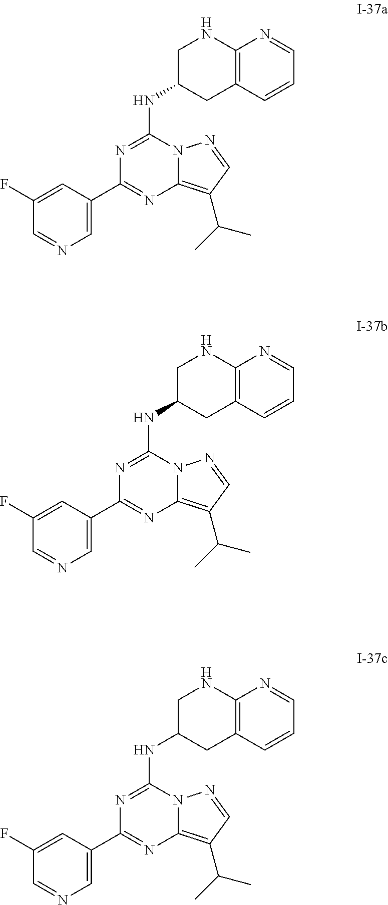 [SMILES: CC(C)c1cnn2c(N[C@@H]3CNc4ncccc4C3)nc(-c3cncc(F)c3)nc12 ; CC(C)c1cnn2c(N[C@H]3CNc4ncccc4C3)nc(-c3cncc(F)c3)nc12 ; CC(C)c1cnn2c(NC3CNc4ncccc4C3)nc(-c3cncc(F)c3)nc12]

### Synthetic Scheme:

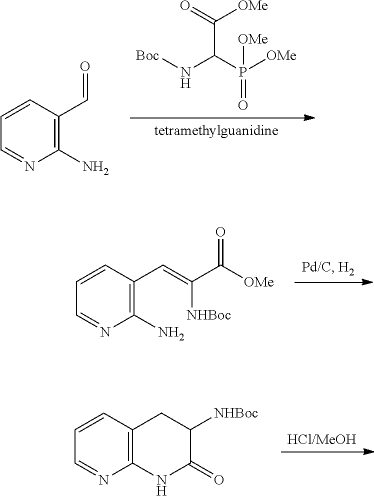

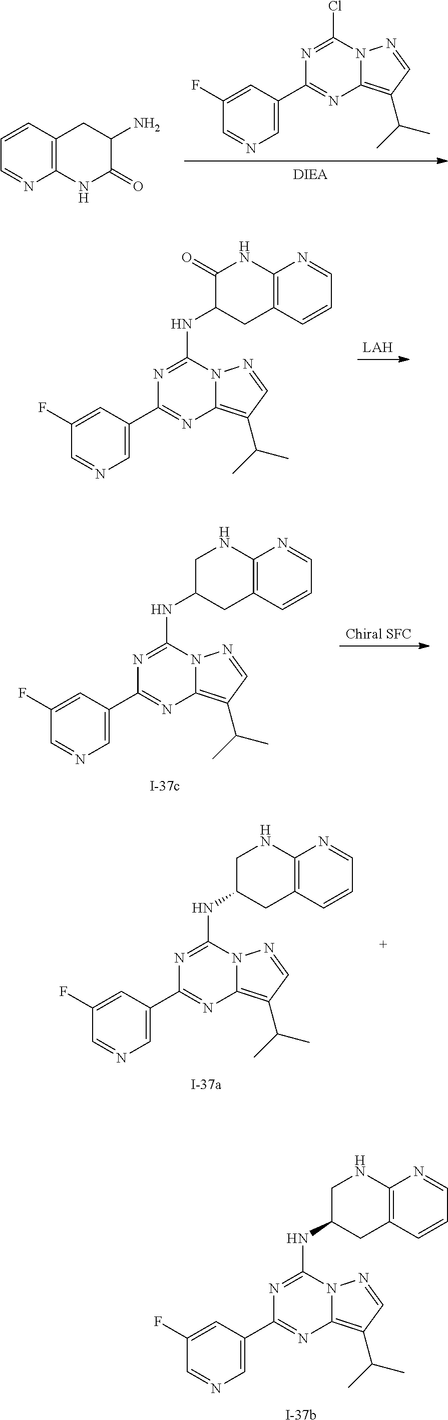

### Step 1: Methyl (Z)-3-(2-amino-3-pyridyl)-2-(tert-butoxycarbonylamino)prop-2-enoate

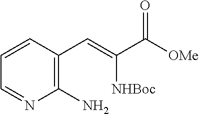 [SMILES: COC(=O)/C(=C/c1cccnc1N)NC(=O)OC(C)(C)C]

To a solution of methyl 2-(tert-butoxycarbonylamino)-2-dimethoxyphosphoryl-acetate (5.48 g, 18.42 mmol, 1 eq) in THE (135 mL) was added tetramethylguanidine (2.23 g, 19.34 mmol, 2.42 mL, 1.05 eq) slowly at −70° C. under N₂. After being stirred for 15 min, a solution of 2-aminopyridine-3-carbaldehyde (2.25 g, 18.42 mmol, 1 eq) in THF (50 mL) was added drop-wise at −70° C. The resulting mixture was stirred for 12 h at 25° C. The reaction mixture was quenched by addition of water (100 mL), extracted with EtOAc (100 mL×3). The combined organic layers were washed with brine (50 mL), dried over Na₂SO₄, filtered and concentrated under reduced pressure to give a residue, which was purified by flash silica gel chromatography (from PE/EtOAc=1/0 to 0/1, TLC: PE/EtOAc=1/1, R_{f}=0.24) to yield methyl (Z)-3-(2-amino-3-pyridyl)-2-(tert-butoxycarbonylamino)prop-2-enoate (3.1 g, 10.04 mmol, 54.5% yield, 95.0% purity) as a yellow solid. ¹H NMR (400 MHz, DMSO-d₆) δ ppm 8.75-8.35 (m, 1H), 7.93 (dd, J=1.7, 4.9 Hz, 1H), 7.64 (dd, J=1.5, 7.6 Hz, 1H), 7.02 (br s, 1H), 6.58 (dd, J=4.9, 7.6 Hz, 1H), 6.10 (s, 2H), 3.72 (s, 3H), 1.39 (s, 9H); ES-LCMS m/z 294.1 [M+H]⁺.

### Step 2: tert-Butyl N-(2-oxo-3,4-dihydro-1H-1,8-naphthyridin-3-yl)carbamate

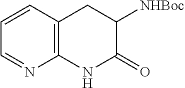 [SMILES: CC(C)(C)OC(=O)NC1Cc2cccnc2NC1=O]

To a solution of methyl (Z)-3-(2-amino-3-pyridyl)-2-(tert-butoxycarbonylamino) prop-2-enoate (2.1 g, 6.80 mmol, 1 eq) in EtOH (60 mL) was added Pd/C (400 mg, 10% purity) under H₂ (30 psi). The mixture was stirred at 25° C. for 24 h. The reaction mixture was filtered and the filtrate was concentrated under reduced pressure to yield tert-butyl N-(2-oxo-3,4-dihydro-1H-1,8-naphthyridin-3-yl)carbamate (1.6 g, crude) as a white solid which was used in the next step without further purification. ¹H NMR (400 MHz, DMSO-d₆) δ ppm 10.61 (br s, 1H), 8.11 (d, J=4.2 Hz, 1H), 7.60 (d, J=7.3 Hz, 1H), 7.11-6.89 (m, 2H), 4.31-4.15 (m, 1H), 3.08-2.84 (m, 2H), 1.41 (s, 9H); ES-LCMS m/z 264.2 [M+H]⁺.

### Step 3: 3-Amino-3,4-dihydro-1H-1,8-naphthyridin-2-one

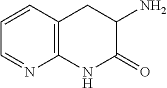 [SMILES: NC1Cc2cccnc2NC1=O]

To a solution of tert-butyl N-(2-oxo-3,4-dihydro-1H-1,8-naphthyridin-3-yl)carbamate (500 mg, 1.90 mmol, 1 eq) in DCM (5 mL) was added HCl/1,4-dioxane (4 M, 5 mL). The mixture was stirred at 25° C. for 12 h. The reaction mixture was concentrated under reduced pressure to yield 3-amino-3,4-dihydro-1H-1,8-naphthyridin-2-one (450 mg, crude, 2HCl) as a white solid which was used in the next step without further purification. ¹H NMR (400 MHz, DMSO-d₆) δ ppm 11.14 (s, 1H), 8.72-8.55 (m, 2H), 8.18 (d, J=4.3 Hz, 1H), 7.73 (d, J=7.5 Hz, 1H), 7.05 (dd, J 4.9, 7.4 Hz, 1H), 4.46-4.27 (m, 1H), 3.26-3.06 (m, 2H).

### Step 4: 3-[[2-(5-Fluoro-3-pyridyl)-8-isopropyl-pyrazolo[1,5-a][1,3,5]triazin-4-yl]amino]-3,4-dihydro-1H-1,8-naphthyridin-2-one (I-37c)

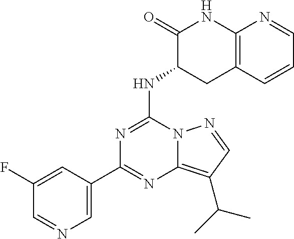 [SMILES: CC(C)c1cnn2c(N[C@H]3Cc4cccnc4NC3=O)nc(-c3cncc(F)c3)nc12]

To a solution of 4-chloro-2-(5-fluoro-3-pyridyl)-8-isopropyl-pyrazolo[1,5-a][1,3,5]triazine (396.74 mg, 1.33 mmol, 0.7 eq) in i-PrOH (20 mL) was added DIEA (985.34 mg, 7.62 mmol, 1.33 mL, 4 eq) and 3-amino-3,4-dihydro-1H-1,8-naphthyridin-2-one (450 mg, 1.91 mmol, 1 eq, 2HCl). The mixture was stirred at 60° C. for 12 h. The reaction mixture was concentrated under reduced pressure to yield a residue which was purified by flash silica gel chromatography (from PE/EtOAc=1/0 to 0/1, TLC: PE/EtOAc=1/1, R_{f}=0.24) to yield 3-[[2-(5-fluoro-3-pyridyl)-8-isopropyl-pyrazolo[1,5-a][1,3,5]triazin-4-yl]amino]-3,4-dihydro-1H-1,8-naphthyridin-2-one (280 mg, 659.14 μmol, 34.5% yield, 98.5% purity) as a white solid. ¹H NMR (400 MHz, CD3OD) δ ppm 9.36 (s, 1H), 8.54-8.41 (m, 2H), 8.21 (d, J=4.6 Hz, 1H), 8.00 (s, 1H), 7.69 (s, 1H), 7.08 (dd, J=5.1, 7.6 Hz, 1H), 5.30 (dd, J=7.0, 14.3 Hz, 1H), 3.57-3.49 (m, 1H), 3.47-3.34 (m, 2H), 1.42 (d, J=6.8 Hz, 6H); ES-LCMS m/z 419.2 [M+H]⁺.

### Step 5: (3S)—N-[2-(5-Fluoro-3-pyridyl)-8-isopropyl-pyrazolo[1,5-a][1,3,5]triazin-4-yl]-1,2,3,4-tetrahydro-1,8-naphthyridin-3-amine (I-37a) & (3R)—N-[2-(5-Fluoro-3-pyridyl)-8-isopropyl-pyrazolo[1,5-a][1,3,5]triazin-4-yl]-1,2,3,4-tetrahydro-1,8-naphthyridin-3-amine (I-37b)

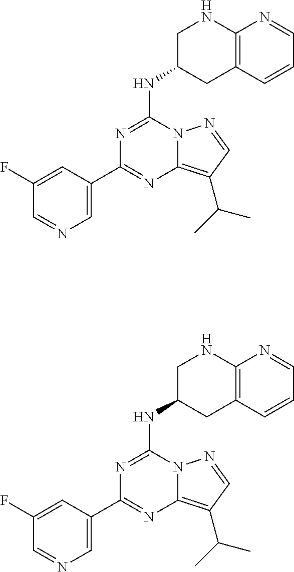 [SMILES: CC(C)c1cnn2c(N[C@@H]3CNc4ncccc4C3)nc(-c3cncc(F)c3)nc12 ; CC(C)c1cnn2c(N[C@H]3CNc4ncccc4C3)nc(-c3cncc(F)c3)nc12]

To a solution of 3-[[2-(5-fluoro-3-pyridyl)-8-isopropyl-pyrazolo[1,5-a][1,3,5]triazin-4-yl] amino]-3,4-dihydro-1H-1,8-naphthyridin-2-one (230 mg, 541.43 μmol, 1 eq) in THE (25 mL) was added LAH (102.75 mg, 2.71 mmol, 5 eq) at 0° C. The mixture was stirred at 0° C. for 2 h. The reaction mixture was diluted with THE (100 mL), quenched by addition of water (0.11 mL), 10% aq. NaOH (0.11 mL), water (0.33 mL) in sequence at 0° C. After being stirred for 30 min, the mixture was filtered and the filtrate was concentrated under reduced pressure to yield a residue which was purified by flash silica gel chromatography (from PE/EtOAc=1/0 to 1/1, TLC: PE/EtOAc=1/1, R_{f}=0.49) to yield a product (150 mg). The product was separated by SFC (Phenomenex-Cellulose-2 (250 mm*30 mm, 10 μm); mobile phase: [0.1% NH₃H₂O MeOH]; B %: 40%-40%). The residues were purified by preparative HPLC (column: Phenomenex Gemini 150*25 mm*10 μm; mobile phase: [water (0.05% HCl)−ACN]; B %: 51%-81%, 10 min), followed by lyophilization to yield an enantiomer (17.52 mg, 34.10 umol, 6.3% yield, 100% purity, 3HCl, SFC: Rₜ=1.914, ee=100%, [α]^{30.0}_{D}=+7.067 (MeOH, c=0.065 g/100 mL)) as a yellow solid. ¹H NMR (400 MHz, CD3OD) δ ppm 9.52 (s, 1H), 8.90-8.83 (m, 1H), 8.80 (d, J=1.7 Hz, 1H), 8.04 (s, 1H), 7.87-7.79 (m, 2H), 6.91 (t, J=6.7 Hz, 1H), 5.10-5.02 (m, 1H), 3.93 (dd, J=2.8, 12.6 Hz, 1H), 3.75 (dd, J=6.7, 12.8 Hz, 1H), 3.42-3.32 (m, 2H), 3.30-3.24 (m, 1H), 1.41 (d, J=6.8 Hz, 6H); ES-LCMS m/z 405.2 [M+H]⁺; and the other enantiomer (18.38 mg, 35.77 umol, 6.6% yield, 100% purity, 3HCl, SFC: Rₜ=1.560, ee=99.2%, [α]^{29.8}_{D}=−17.487 (MeOH, c=0.060 g/100 mL)) as a yellow solid. ¹H NMR (400 MHz, CD3OD) δ ppm 9.49 (s, 1H), 8.80-8.70 (m, 2H), 8.03 (s, 1H), 7.88-7.77 (m, 2H), 6.91 (t, J=6.8 Hz, 1H), 5.09-5.00 (m, 1H), 3.97-3.88 (m, 1H), 3.75 (dd, J=7.0, 12.8 Hz, 1H), 3.43-3.32 (m, 2H), 3.30-3.24 (m, 1H), 1.41 (d, J=7.1 Hz, 6H); ES-LCMS m/z 405.2 [M+H]⁺.

### Example 41

### Synthesis of Compound I-38

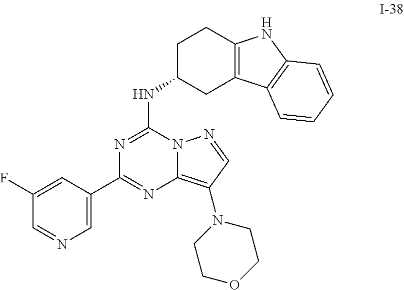 [SMILES: Fc1cncc(-c2nc(N[C@@H]3CCc4[nH]c5ccccc5c4C3)n3ncc(N4CCOCC4)c3n2)c1]

### Synthetic Scheme:

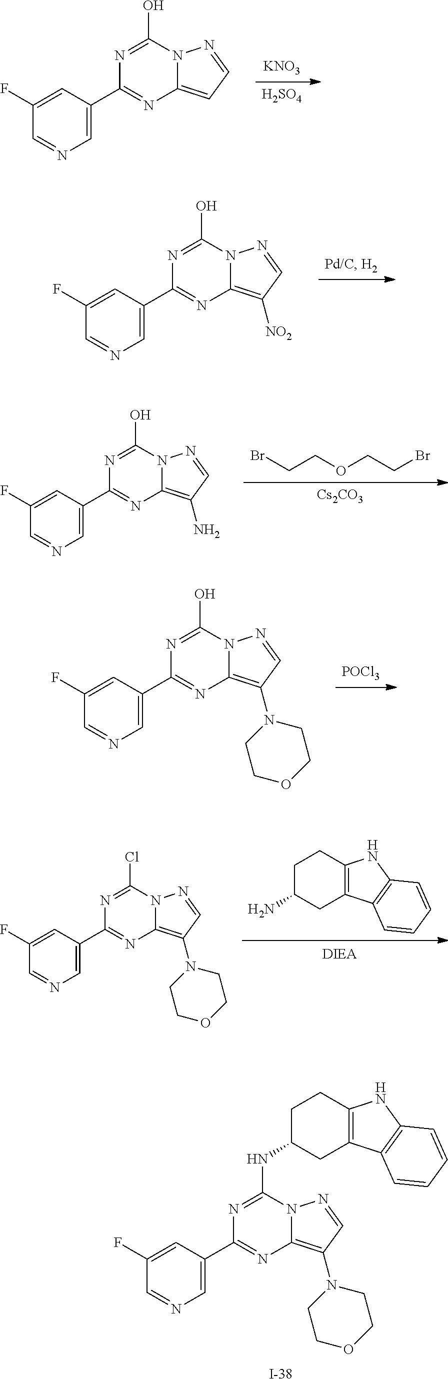

### Step 1: 2-(5-Fluoro-3-pyridyl)-8-nitro-pyrazolo[1,5-a][1,3,5]triazin-4-ol

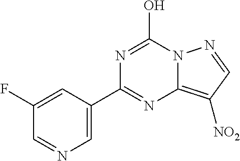 [SMILES: O=[N+]([O-])c1cnn2c(O)nc(-c3cncc(F)c3)nc12]

To a mixture of 2-(5-fluoro-3-pyridyl)pyrazolo[1,5-a][1,3,5]triazin-4-ol (9.2 g, 32.95 mmol, 1 eq) in H₂SO₄ (90 mL) was added KNO₃ (9.99 g, 98.85 mmol, 3 eq) partwise at 0° C. The mixture was stirred at 25° C. for 12 h. TLC (EtOAc, R_{f}=0.05) showed the starting material was consumed completely. The reaction mixture was poured into ice-water (500 mL), adjusted to pH˜3 by NaOH solid and filtered. The solid was washed with water (100 mL), dried under reduced pressure to yield 2-(5-fluoro-3-pyridyl)-8-nitro-pyrazolo[1,5-a][1,3,5]triazin-4-ol (8 g, 21.38 mmol, 64.9% yield, 100.0% purity, H₂SO₄) as a yellow solid which was used in the next step without further purification. ¹H NMR (400 MHz, DMSO-d₆) δ ppm 9.20 (s, 1H), 8.84 (d, J=2.8 Hz, 1H), 8.74 (s, 1H), 8.38-8.34 (m, 1H); ES-LCMS m/z 277.1 [M+H]⁺.

### Step 2: 8-Amino-2-(5-fluoro-3-pyridyl)pyrazolo[1,5-a][1,3,5]triazin-4-ol

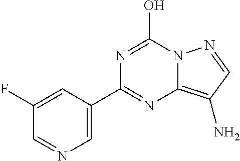 [SMILES: Nc1cnn2c(O)nc(-c3cncc(F)c3)nc12]

A mixture of 2-(5-fluoro-3-pyridyl)-8-nitro-pyrazolo[1,5-a][1,3,5]triazin-4-ol (8 g, 28.97 mmol, 1 eq) and Pd/C (1 g, 10% purity) in MeOH (80 mL) and THE (80 mL) was stirred under H₂ (30 Psi) at 25° C. for 12 h. To the mixture was added water (300 mL). The mixture was acidified with aqueous HCl (2 M) until pH=3 and filtered. The filtrate was concentrated under reduced pressure to yield 8-amino-2-(5-fluoro-3-pyridyl)pyrazolo[1,5-a][1,3,5]triazin-4-ol (7.5 g, 26.27 mmol, 90.7% yield, 99.0% purity, HCl) as an off-white solid which was used in the next step without further purification. ¹H NMR (400 MHz, DMSO-d₆) δ ppm 9.16 (s, 1H), 8.83 (d, J=2.8 Hz, 1H), 8.37 (td, J=2.4, 9.6 Hz, 1H), 8.22 (s, 1H); ES-LCMS m/z 247.2 [M+H]⁺.

### Step 3: 2-(5-Fluoro-3-pyridyl)-8-morpholino-pyrazolo[1,5-a][1,3,5]triazin-4-ol

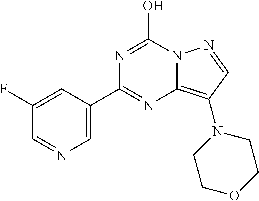 [SMILES: Oc1nc(-c2cncc(F)c2)nc2c(N3CCOCC3)cnn12]

To a stirred solution of 8-amino-2-(5-fluoro-3-pyridyl)pyrazolo[1,5-a][1,3,5]triazin-4-ol (500 mg, 1.75 mmol, 1 eq, HCl) in DMF (30 mL) was added Cs₂CO₃ (2.85 g, 8.76 mmol, 5 eq) and 1-bromo-2-(2-bromoethoxy)ethane (446.74 mg, 1.93 mmol, 241.48 μL, 1.1 eq). The reaction mixture was stirred at 80° C. for 12 h. The reaction mixture was concentrated to yield a residue which was purified by flash silica gel chromatography (from DCM/MeOH=100/1 to 10/1, TLC: DCM/MeOH=10/1, R_{f}=0.15) to yield 2-(5-fluoro-3-pyridyl)-8-morpholino-pyrazolo[1,5-a][1,3,5]triazin-4-ol (400 mg, 862.50 μmol, 49.3% yield, 68.2% purity) as a brown oil. ¹H NMR (400 MHz, CD3OD) δ ppm 9.11 (s, 1H), 8.64 (d, J=2.7 Hz, 1H), 8.30-8.26 (m, 1H), 7.89 (s, 1H), 3.89-3.85 (m, 4H), 3.39-3.35 (m, 4H); ES-LCMS m/z 317.2 [M+H]⁺.

### Step 4: 4-[4-Chloro-2-(5-fluoro-3-pyridyl)pyrazolo[1,5-a][1,3,5]triazin-8-yl]morpholine

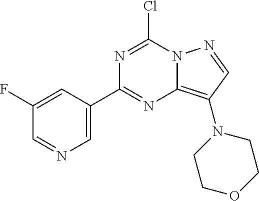 [SMILES: Fc1cncc(-c2nc(Cl)n3ncc(N4CCOCC4)c3n2)c1]

To a stirred solution of 2-(5-fluoro-3-pyridyl)-8-morpholino-pyrazolo[1,5-a][1,3,5]triazin-4-ol (400 mg, 862.50 μmol, 1 eq) in toluene (5 mL) was added DIEA (334.41 mg, 2.59 mmol, 450.69 μL, 3 eq) and POCl₃ (1.32 g, 8.62 mmol, 801.50 μL, 10 eq). The reaction mixture was stirred at 120° C. for 2 h. The reaction mixture was concentrated to yield 4-[4-chloro-2-(5-fluoro-3-pyridyl)pyrazolo[1,5-a][1,3,5]triazin-8-yl]morpholine (300 mg, 390.77 μmol, 45.3% yield, 53.1% purity, 2HCl) as brown oil which was used in the next step without further purification. ES-LCMS m/z 335.1, 337.1 [M+H]⁺.

### Step 5: (3R)—N-[2-(5-Fluoro-3-pyridyl)-8-morpholino-pyrazolo[1,5-a][1,3,5]triazin-4-yl]-2,3,4,9-tetrahydro-1H-carbazol-3-amine (I-38)

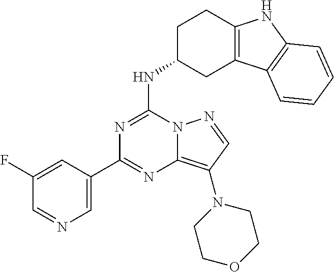 [SMILES: Fc1cncc(-c2nc(N[C@@H]3CCc4[nH]c5ccccc5c4C3)n3ncc(N4CCOCC4)c3n2)c1]

To a solution of 4-[4-chloro-2-(5-fluoro-3-pyridyl)pyrazolo[1,5-a][1,3,5]triazin-8-yl]morpholine (300 mg, 390.77 μmol, 1 eq, 2HCl) in ACN (5 mL) was added DIEA (252.52 mg, 1.95 mmol, 340.32 μL, 5 eq) and (3R)-2,3,4,9-tetrahydro-1H-carbazol-3-amine (72.78 mg, 390.77 μmol, 1 eq). The mixture was stirred at 60° C. for 1 h. The reaction mixture was concentrated to yield a residue which was purified by preparative HPLC (HCl condition; column: Agela ASB 150*25 mm*5 μm; mobile phase: [water (0.05% HCl)−ACN]; B %: 53%-83%, 8 min). The desired fraction was lyophilized to yield (3R)—N-[2-(5-fluoro-3-pyridyl)-8-morpholino-pyrazolo[1,5-a][1,3,5]triazin-4-yl]-2,3,4,9-tetrahydro-1H-carbazol-3-amine (29.64 mg, 49.91 μmol, 12.8% yield, 100.0% purity, 3HCl, [α]^{29.0}_{D}=+5.657 (DMSO, c=0.045 g/100 mL)) as a yellow solid. ¹H NMR (400 MHz, CD3OD) δ ppm 9.41 (s, 1H), 8.63 (s, 2H), 8.07 (s, 1H), 7.38 (d, J=7.6 Hz, 1H), 7.28 (d, J=8.1 Hz, 1H), 7.07-7.01 (m, 1H), 6.99-6.93 (m, 1H), 4.92 (m, 1H), 3.99 (m, 4H), 3.72-3.45 (m, 4H), 3.28 (d, J=5.1 Hz, 1H), 3.11-2.91 (m, 3H), 2.39 (d, J=9.8 Hz, 1H), 2.32-2.21 (m, 1H); ES-LCMS m/z 485.3 [M+H]⁺.

### Example 42

### Synthesis of Compound I-39

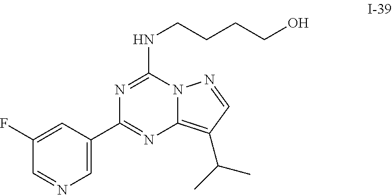 [SMILES: CC(C)c1cnn2c(NCCCCO)nc(-c3cncc(F)c3)nc12]

### Synthetic Scheme:

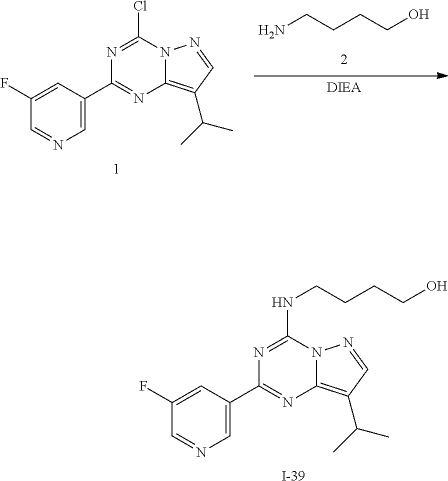

### Step 1: 4-[[2-(5-Fluoro-3-pyridyl)-8-isopropyl-pyrazolo[1,5-a][1,3,5]triazin-4-yl]amino]butan-1-ol (I-39)

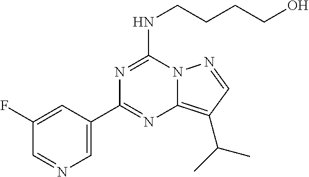 [SMILES: CC(C)c1cnn2c(NCCCCO)nc(-c3cncc(F)c3)nc12]

To a solution of 4-chloro-2-(5-fluoro-3-pyridyl)-8-isopropyl-pyrazolo[1,5-a][1,3,5]triazine (50 mg, 168.15 μmol, 1 eq) in i-PrOH (3 mL) was added DIEA (65.19 mg, 504.44 μmol, 87.86 μL, 3 eq) and 4-aminobutan-1-ol (29.98 mg, 336.29 μmol, 31.22 μL, 2 eq). The mixture was stirred at 90° C. for 12 h. The reaction mixture was concentrated under reduced pressure to yield a residue which was purified by preparative HPLC (column: Agela ASB 150*25 mm*5 μm; mobile phase: [water (0.05% HCl)−ACN]; B %: 45%-75%, 8 min), followed by lyophilization to yield 4-[[2-(5-fluoro-3-pyridyl)-8-isopropyl-pyrazolo[1,5-a][1,3,5]triazin-4-yl]amino]butan-1-ol (38.4 mg, 92.02 μmol, 54.7% yield, 100% purity, 2HCl) as a yellow solid. ¹H NMR (400 MHz, CD3OD) δ ppm 9.42 (s, 1H), 8.61-8.43 (m, 2H), 7.96 (s, 1H), 3.80 (t, J=7.0 Hz, 2H), 3.64 (t, J=6.5 Hz, 2H), 3.29-3.23 (m, 1H), 1.94-1.82 (m, 2H), 1.75-1.64 (m, 2H), 1.41 (d, J=7.1 Hz, 6H); ES-LCMS m/z 345.2 [M+H]⁺.

### Example 43

### Synthesis of Compound I-40

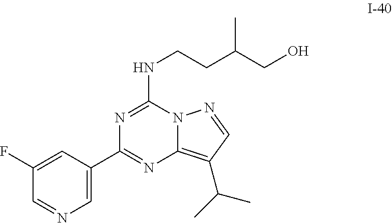 [SMILES: CC(CO)CCNc1nc(-c2cncc(F)c2)nc2c(C(C)C)cnn12]

### Synthetic Scheme:

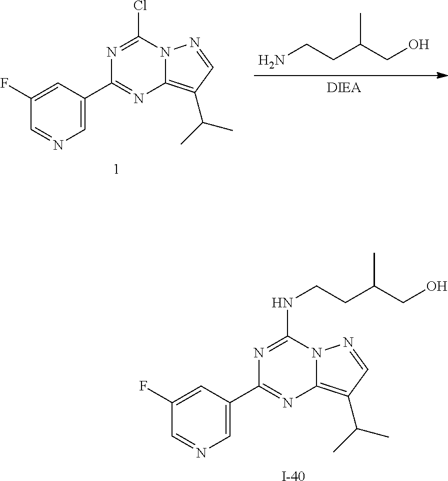

### Step 1: 4-[[2-(5-Fluoro-3-pyridyl)-8-isopropyl-pyrazolo[1,5-a][1,3,5]triazin-4-yl]amino]-2-methyl-butan-1-ol (I-40)

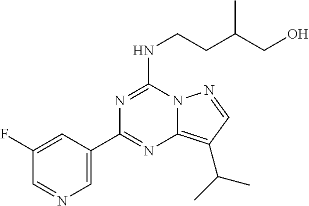 [SMILES: CC(CO)CCNc1nc(-c2cncc(F)c2)nc2c(C(C)C)cnn12]

To a solution of 4-chloro-2-(5-fluoro-3-pyridyl)-8-isopropyl-pyrazolo[1,5-a][1,3,5]triazine (50.97 mg, 171.40 μmol, 1 eq) in ACN (5 mL) was added DIEA (110.76 mg, 857.01 μmol, 149.27 μL, 5 eq) and 4-amino-2-methyl-butan-1-ol (18.57 mg, 179.97 μmol, 1.05 eq). The mixture was stirred at 60° C. for 1 h. The reaction mixture was concentrated to yield a residue which was purified by preparative HPLC (HCl condition; column: Agela ASB 150*25 mm*5 μm; mobile phase: [water (0.05% HCl)−ACN]; B %: 50%-80%, 8 min). The desired fraction was lyophilized to yield 4-[[2-(5-fluoro-3-pyridyl)-8-isopropyl-pyrazolo[1,5-a][1,3,5]triazin-4-yl]amino]-2-methyl-butan-1-ol (44.62 mg, 103.45 μmol, 60.4% yield, 100.0% purity, 2HCl) as a yellow solid. ¹H NMR (400 MHz, CD3OD) δ ppm 9.43 (s, 1H), 8.62-8.54 (m, 2H), 7.96 (s, 1H), 3.90-3.75 (m, 2H), 3.48 (d, J=6.1 Hz, 2H), 3.30-3.23 (m, 1H), 2.02-1.90 (m, 1H), 1.85-1.72 (m, 1H), 1.65-1.54 (m, 1H), 1.41 (d, J=6.8 Hz, 6H), 1.05 (d, J=6.8 Hz, 3H); ES-LCMS m/z 359.2 [M+H]⁺.

### Example 44

### Synthesis of Compound I-41a, I-41b and I-41c

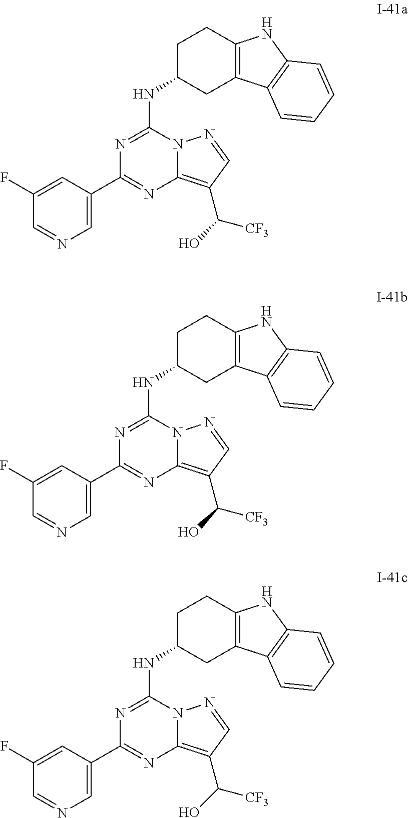 [SMILES: O[C@H](c1cnn2c(N[C@@H]3CCc4[nH]c5ccccc5c4C3)nc(-c3cncc(F)c3)nc12)C(F)(F)F ; O[C@@H](c1cnn2c(N[C@@H]3CCc4[nH]c5ccccc5c4C3)nc(-c3cncc(F)c3)nc12)C(F)(F)F ; OC(c1cnn2c(N[C@@H]3CCc4[nH]c5ccccc5c4C3)nc(-c3cncc(F)c3)nc12)C(F)(F)F]

### Synthetic Scheme:

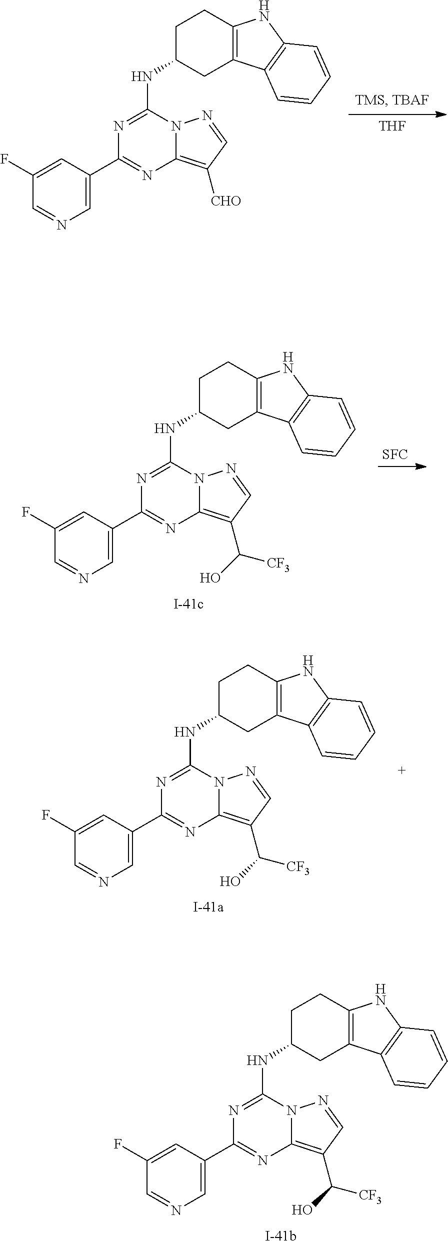

### Step 1: (1R)-2,2,2-Trifluoro-1-[2-(5-fluoro-3-pyridyl)-4-[[(3R)-2,3,4,9-tetrahydro-1H-carbazol-3-yl]amino]pyrazolo[1,5-a][1,3,5]triazin-8-yl]ethanol (I-41a) & (1S)-2,2,2-trifluoro-1-[2-(5-fluoro-3-pyridyl)-4-[[(3R)-2,3,4,9-tetrahydro-1H-carbazol-3-yl]amino]pyrazolo[1,5-a][1,3,5]triazin-8-yl]ethanol (I-41b)

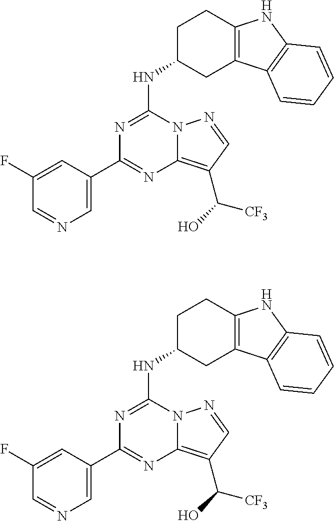 [SMILES: O[C@H](c1cnn2c(N[C@@H]3CCc4[nH]c5ccccc5c4C3)nc(-c3cncc(F)c3)nc12)C(F)(F)F ; O[C@@H](c1cnn2c(N[C@@H]3CCc4[nH]c5ccccc5c4C3)nc(-c3cncc(F)c3)nc12)C(F)(F)F]

To a solution of 2-(5-fluoro-3-pyridyl)-4-[[(3R)-2,3,4,9-tetrahydro-1H-carbazol-3-yl]amino] pyrazolo[1,5-a][1,3,5]triazine-8-carbaldehyde (700 mg, 1.54 mmol, 1 eq) in THE (14 mL) was cooled to 0° C. and added TBAF (1 M, 11.81 mL, 7.67 eq). Then TMS-CF₃ (3.79 g, 26.62 mmol, 3.94 mL, 17.29 eq) was added to the above mixture at 0° C. The mixture was warmed to 28° C. and stirred for 24 h. The reaction mixture was added to water (50 mL) and extracted with EtOAc (50 mL×3). The combined organic layers were washed with brine (20 mL), dried over Na₂SO₄ and concentrated to yield a residue which was purified by preparative HPLC (basic condition; column: Boston Prime C18 150*30 mm Sum; mobile phase: [water (0.05% ammonia hydroxide v/v)−ACN]; B %: 50%-70%, 9 min) to yield an enantiomer as pure (24.44 mg, 46.72 μmol, 3.0% yield, 95.1% purity, SFC: Rₜ=5.190 min, ee=97.4%, [α]^{28.7}_{D}=+33.394, MeOH, c=0.044 g/100 mL) as a white solid; ¹H NMR (400 MHz, CDCl₃) δ ppm 9.48 (s, 1H), 8.59 (d, J=3.0 Hz, 1H), 8.40 (td, J=2.2, 9.4 Hz, 1H), 8.04 (s, 1H), 7.92 (s, 1H), 7.48 (d, J=7.8 Hz, 1H), 7.36 (d, J=8.0 Hz, 1H), 7.20 (t, J=7.3 Hz, 1H), 7.16-7.10 (m, 1H), 6.88 (d, J=7.8 Hz, 1H), 5.42 (q, J=6.4 Hz, 1H), 4.99 (m, 1H), 4.28 (m, 1H), 3.38 (dd, J=5.4, 15.2 Hz, 1H), 3.08-2.93 (m, 3H), 2.43-2.30 (m, 2H); ES-LCMS m/z 498.2 [M+H]⁺ and the other enantiomer as crude which was re-purified by preparative TLC (PE/EtOAc=2/1, TLC: PE/EtOAc=2/1, R_{f}=0.50) to yield the other enantiomer as pure (12.28 mg, 23.75 μmol, 1.5% yield, 96.2% purity, SFC: Rₜ=5.839 min, ee=100.0%, [α]^{28.6}_{D}=+23.768, MeOH, c=0.042 g/100 mL) as a white solid; ¹H NMR (400 MHz, CDCl₃) δ ppm 9.48 (s, 1H), 8.60 (d, J=2.8 Hz, 1H), 8.43-8.37 (m, 1H), 8.05 (s, 1H), 7.90 (s, 1H), 7.48 (d, J=7.5 Hz, 1H), 7.36 (d, J=8.0 Hz, 1H), 7.22-7.17 (m, 1H), 7.16-7.11 (m, 1H), 6.86 (d, J=8.5 Hz, 1H), 5.43 (t, J=6.8 Hz, 1H), 4.99 (m, 1H), 4.08 (d, J=7.0 Hz, 1H), 3.39 (dd, J=4.9, 15.4 Hz, 1H), 3.09-2.93 (m, 3H), 2.43-2.33 (m, 2H); ES-LCMS m/z 498.2 [M+H]⁺.

### Example 45

### Synthesis of Compound I-42a and I-42b

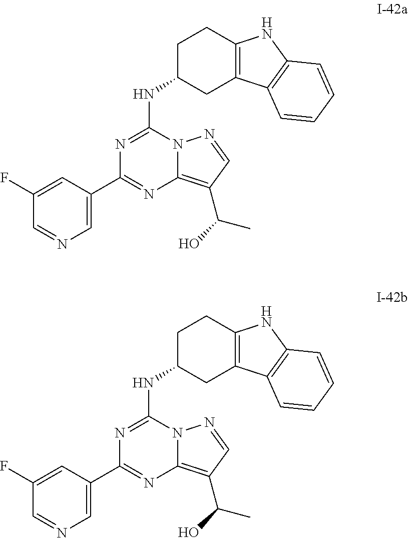 [SMILES: C[C@H](O)c1cnn2c(N[C@@H]3CCc4[nH]c5ccccc5c4C3)nc(-c3cncc(F)c3)nc12 ; C[C@@H](O)c1cnn2c(N[C@@H]3CCc4[nH]c5ccccc5c4C3)nc(-c3cncc(F)c3)nc12]

### Synthetic Scheme:

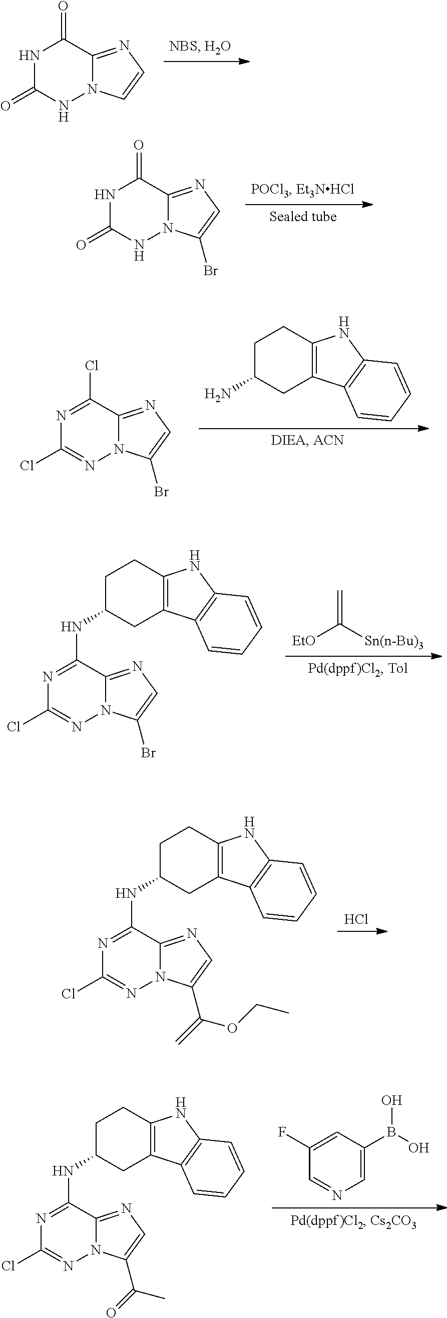

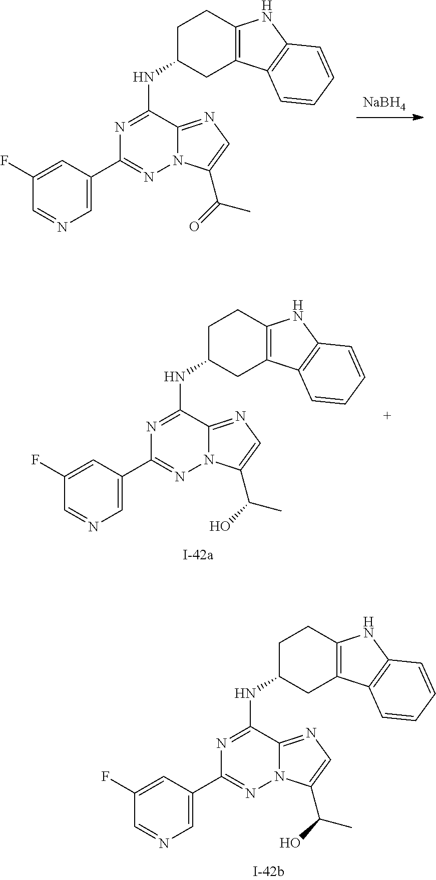 [SMILES: CC(=O)c1cnc2c(N[C@@H]3CCc4[nH]c5ccccc5c4C3)nc(-c3cncc(F)c3)nn12 ; "NaBH4" ; C[C@H](O)c1cnc2c(N[C@@H]3CCc4[nH]c5ccccc5c4C3)nc(-c3cncc(F)c3)nn12 ; C[C@@H](O)c1cnc2c(N[C@@H]3CCc4[nH]c5ccccc5c4C3)nc(-c3cncc(F)c3)nn12]

### Step 1: 7-Bromo-1H-imidazo[2,1-f][1,2,4]triazine-2,4-dione

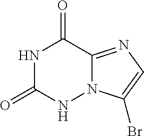 [SMILES: O=c1[nH]c(=O)c2ncc(Br)n2[nH]1]

To a solution of 1H-imidazo[2,1-f][1,2,4]triazine-2,4-dione (1.64 g, 9.70 mmol, 1 eq) in H₂O (40 mL) was added NBS (1.21 g, 6.79 mmol, 0.7 eq) at 0° C. The reaction mixture was stirred at 25° C. for 1 h. The reaction mixture was concentrated. MeOH (30 mL) was added and stirred for 1 h. The mixture was filtered and the residue was concentrated to yield 7-bromo-1H-imidazo[2,1-f][1,2,4]triazine-2,4-dione (2.3 g, 8.96 mmol, 92.4% yield, 90.0% purity) as a white solid. ¹H NMR (400 MHz, DMSO-d₆) δ ppm 7.20-7.06 (m, 1H); ES-LCMS m/z 231.1, 233.1 [M+H]⁺.

### Step 2: 7-Bromo-2,4-dichloro-imidazo[2,1-f][1,2,4]triazine

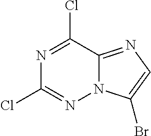 [SMILES: Clc1nc(Cl)c2ncc(Br)n2n1]

A mixture of 7-bromo-1H-imidazo[2,1-f][1,2,4]triazine-2,4-dione (2.3 g, 8.96 mmol, 1 eq), Et₃N HCl (2.47 g, 17.92 mmol, 2 eq) and POCl₃ (132.00 g, 860.89 mmol, 80 mL, 96.07 eq) was stirred in a sealed tube at 135° C. for 24 h. The reaction mixture was concentrated and to the residue was added DCM (60 mL). The mixture was poured into ice cold water (20 mL), extracted with DCM (60 mL×2). The combined organic layers were washed with brine (20 mL), dried over Na₂SO₄, filtered and concentrated to yield 7-bromo-2,4-dichloro-imidazo[2,1-f][1,2,4]triazine (2.8 g, 8.36 mmol, 93.3% yield, 80.0% purity) as a brown solid which was used in the next step without purification. ¹H NMR (400 MHz, CDCl₃) δ ppm 8.01 (s, 1H); ES-LCMS m/z 267.0, 269.0, 271.0 [M+H]⁺.

### Step 3: (3R)—N-(7-Bromo-2-chloro-imidazo[2,1-f][1,2,4]triazin-4-yl)-2,3,4,9-tetrahydro-1H-carbazol-3-amine

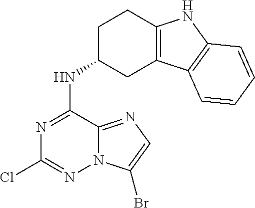 [SMILES: Clc1nc(N[C@@H]2CCc3[nH]c4ccccc4c3C2)c2ncc(Br)n2n1]

A mixture of 7-bromo-2,4-dichloro-imidazo[2,1-f][1,2,4]triazine (1.5 g, 4.48 mmol, 1 eq), (3R)-2,3,4,9-tetrahydro-1H-carbazol-3-amine (834.28 mg, 4.48 mmol, 1 eq) and DIEA (2.32 g, 17.92 mmol, 3.12 mL, 4 eq) in CH₃CN (40 mL) was stirred at 80° C. for 2 h. The reaction mixture was concentrated to yield a residue which was purified on silica gel column chromatography (from PE/EtOAc=5/1 to 3/1, TLC: PE/EtOAc=3/1, R_{f}=0.30) to yield (3R)—N-(7-bromo-2-chloro-imidazo[2,1-f][1,2,4]triazin-4-yl)-2,3,4,9-tetrahydro-1H-carbazol-3-amine (1.3 g, 2.72 mmol, 60.8% yield, 87.5% purity) as a yellow solid. ¹H NMR (400 MHz, CDCl₃) δ ppm 7.85 (s, 1H), 7.48-7.42 (m, 2H), 7.32 (d, J=7.8 Hz, 1H), 7.17 (dt, J=1.1, 7.5 Hz, 1H), 7.14-7.09 (m, 1H), 6.79 (d, J=8.5 Hz, 1H), 4.94-4.85 (m, 1H), 3.29 (dd, J=5.1, 15.4 Hz, 1H), 2.99-2.84 (m, 3H), 2.30-2.23 (m, 2H); ES-LCMS m/z 417.0, 419.0 [M+H]⁺.

### Step 4: (3R)—N-[2-Chloro-7-(1-ethoxyvinyl)imidazo[2,1-f][1,2,4]triazin-4-yl]-2,3,4,9-tetrahydro-1H-carbazol-3-amine

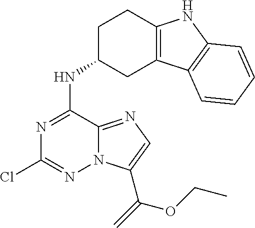 [SMILES: C=C(OCC)c1cnc2c(N[C@@H]3CCc4[nH]c5ccccc5c4C3)nc(Cl)nn12]

To a mixture of (3R)—N-(7-bromo-2-chloro-imidazo[2,1-f][1,2,4]triazin-4-yl)-2,3,4,9-tetrahydro-1H-carbazol-3-amine (500 mg, 1.05 mmol, 1 eq) and tributyl(1-ethoxyvinyl)stannane (945.69 mg, 2.62 mmol, 883.83 μL, 2.5 eq) in toluene (20 mL) was added Pd(dppf)Cl₂ (153.28 mg, 209.49 μmol, 0.2 eq) under N₂ atmosphere. The mixture was stirred at 120° C. for 24 h. The mixture was concentrated to yield a residue which was purified on silica gel column chromatography (from PE/EtOAc=5/1 to 2/1 to PE/EtOAc/DCM=1/1/1, TLC: PE/EtOAc=3/1, R_{f}=0.36) to yield (3R)—N-[2-chloro-7-(1-ethoxyvinyl)imidazo[2,1-f][1,2,4]triazin-4-yl]-2,3,4,9-tetrahydro-1H-carbazol-3-amine (160 mg, 313.05 μmol, 29.9% yield, 80.0% purity) as a yellow solid. ¹H NMR (400 MHz, CDCl₃) δ ppm 7.99 (br s, 1H), 7.57 (s, 1H), 7.37 (dd, J=7.7, 14.6 Hz, 2H), 7.25-7.14 (m, 2H), 7.13-6.96 (m, 3H), 4.77 (d, J=6.8 Hz, 1H), 3.88 (q, J=6.8 Hz, 2H), 3.52-3.42 (m, 1H), 3.30-3.09 (m, 2H), 2.86-2.71 (m, 3H), 1.33 (t, J=7.0 Hz, 3H); ES-LCMS m z 409.2 [M+H]⁺.

### Step 5: 1-[2-Chloro-4-[[(3R)-2,3,4,9-tetrahydro-1H-carbazol-3-yl]amino]imidazo[2,1-f][1,2,4]triazin-7-yl]ethanone

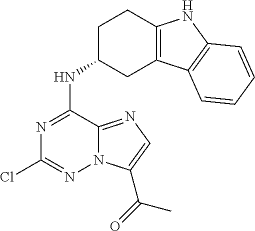 [SMILES: CC(=O)c1cnc2c(N[C@@H]3CCc4[nH]c5ccccc5c4C3)nc(Cl)nn12]

To a solution of (3R)—N-[2-chloro-7-(1-ethoxyvinyl)imidazo[2,1-f][1,2,4]triazin-4-yl]-2,3,4,9-tetrahydro-1H-carbazol-3-amine (300 mg, 586.96 μmol, 1 eq) in DCM (15 mL) was added HCl/MeOH (4 M, 1 mL, 6.81 eq). The mixture was stirred at 25° C. for 1 h. TLC (PE/EtOAc=2/1, R_{f}=0.35) showed the reaction was completed. The mixture was concentrated. To the crude material was added EtOAc (20 mL) and neutralized with saturated aqueous Na₂CO₃ solution to pH=7-8. The mixture was extracted with EtOAc (20 mL×2). The combined organic layers were washed with brine (50 mL), dried over Na₂SO₄, filtered and concentrated to yield 1-[2-chloro-4-[[(3R)-2,3,4,9-tetrahydro-1H-carbazol-3-yl]amino]imidazo[2,1-f][1,2,4]triazin-7-yl]ethanone (250 mg, 525.17 μmol, 89.5% yield, 80.0% purity) as a yellow solid. ¹H NMR (400 MHz, CDCl₃) δ ppm 8.00 (s, 1H), 7.80 (br s, 1H), 7.38 (d, J=7.6 Hz, 1H), 7.25 (d, J=7.8 Hz, 1H), 7.10 (t, J=7.1 Hz, 1H), 7.07-7.01 (m, 1H), 6.97 (d, J=8.1 Hz, 1H), 4.82 (br s, 1H), 3.23 (dd, J=4.9, 15.7 Hz, 1H), 2.96-2.77 (m, 3H), 2.68 (s, 3H), 2.24-2.16 (m, 2H); ES-LCMS m/z 381.1 [M+H]⁺.

### Step 6: 1-[2-(5-Fluoro-3-pyridyl)-4-[[(3R)-2,3,4,9-tetrahydro-1H-carbazol-3-yl]amino]imidazo[2,1-f][1,2,4]triazin-7-yl]ethanone

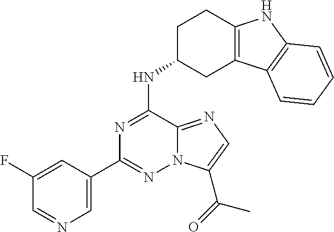 [SMILES: CC(=O)c1cnc2c(N[C@@H]3CCc4[nH]c5ccccc5c4C3)nc(-c3cncc(F)c3)nn12]

To a mixture of 1-[2-chloro-4-[[(3R)-2,3,4,9-tetrahydro-1H-carbazol-3-yl]amino]imidazo[2,1-f][1,2,4]triazin-7-yl]ethanone (230 mg, 483.15 μmol, 1 eq) and (5-fluoro-3-pyridyl)boronic acid (204.24 mg, 1.45 mmol, 3 eq) in 1,4-dioxane (12 mL) and H₂O (3 mL) was added Cs₂CO₃ (472.26 mg, 1.45 mmol, 3 eq) and Pd(dppf)Cl₂ (17.68 mg, 24.16 μmol, 0.05 eq) under N₂ atmosphere. The mixture was irradiated and stirred at 110° C. for 1 h under microwave. TLC (PE/EtOAc=1/1, R_{f}=0.35) showed the reaction was completed. To the mixture was added H₂O (5 mL), extracted with EA (30 mL×3). The combined organic layers were concentrated to yield a residue was purified with preparative TLC (PE/EtOAc=1/1, R_{f}=0.35) to afford 1-[2-(5-fluoro-3-pyridyl)-4-[[(3R)-2,3,4,9-tetrahydro-1H-carbazol-3-yl]amino]imidazo[2,1-f][1,2,4]triazin-7-yl]ethanone (130 mg, 235.58 μmol, 48.8% yield, 80% purity) as a yellow solid. ¹H NMR (400 MHz, CDCl₃) δ ppm 9.36 (s, 1H), 8.53 (d, J=2.7 Hz, 1H), 8.30-8.22 (m, 1H), 8.08 (s, 1H), 7.89-7.79 (m, 1H), 7.40-7.38 (m, 1H), 7.27 (d, J=8.1 Hz, 1H), 7.13-7.08 (m, 1H), 7.07-7.01 (m, 1H), 6.91 (d, J=8.1 Hz, 1H), 4.96 (br s, 1H), 3.29 (dd, J=4.4, 15.2 Hz, 1H), 2.97-2.84 (m, 3H), 2.81 (s, 3H), 2.33-2.20 (m, 2H); ES-LCMS m/z 442.2 [M+H]⁺.

### Step 7: (1S)-1-[2-(5-Fluoro-3-pyridyl)-4-[[(3R)-2,3,4,9-tetrahydro-1H-carbazol-3-yl]amino]imidazo[2,1-f][1,2,4]triazin-7-yl]ethanol (I-42a) and (1R)-1-[2-(5-Fluoro-3-pyridyl)-4-[[(3R)-2,3,4,9-tetrahydro-1H-carbazol-3-yl]amino]imidazo[2,1-f][1,2,4]triazin-7-yl]ethanol (I-42b)

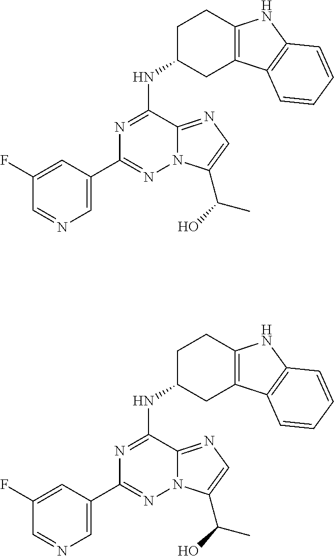 [SMILES: C[C@H](O)c1cnc2c(N[C@@H]3CCc4[nH]c5ccccc5c4C3)nc(-c3cncc(F)c3)nn12 ; C[C@@H](O)c1cnc2c(N[C@@H]3CCc4[nH]c5ccccc5c4C3)nc(-c3cncc(F)c3)nn12]

To a solution of 1-[2-(5-fluoro-3-pyridyl)-4-[[(3R)-2,3,4,9-tetrahydro-1H-carbazol-3-yl] amino]imidazo[2,1-f][1,2,4]triazin-7-yl]ethanone (130 mg, 235.58 μmol, 1 eq) in MeOH (10 mL) was added NaBH₄ (71.30 mg, 1.88 mmol, 8 eq) in portions. The mixture was stirred at 25° C. for 15 min. TLC (PE/EtOAc=1/1, R_{f}=0.3) showed the reaction was completed. The mixture was quenched with H₂O (20 mL), extracted with EtOAc (30 mL×3). The combined organic layers were washed with brine (50 mL), dried over Na₂SO₄, filtered and concentrated to yield a residue which was purified by preparative HPLC (column: Boston Prime C18 150*30 mm 5 μm; mobile phase: [water (0.04% NH₃.H₂O+10 mM NH₄HCO₃)−ACN]; B %: 50%-80%, 8 min), followed by lyophilization to yield an enantiomer (18.53 mg, 41.78 μmol, 17.8% yield, 100% purity, SFC: Rₜ=4.780, ee=99.96%, [α]^{30.1}_{D}=−88.261 (CHCl₃, c=0.030 g/100 mL)) as a white solid. ¹H NMR (400 MHz, CDCl₃) δ ppm 9.33 (s, 1H), 8.55 (d, J=2.5 Hz, 1H), 8.27 (d, J=9.5 Hz, 1H), 7.93 (br s, 1H), 7.49-7.40 (m, 2H), 7.32 (d, J=8.0 Hz, 1H), 7.16 (t, J=7.3 Hz, 1H), 7.12-7.06 (m, 1H), 7.02 (d, J=7.8 Hz, 1H), 5.41 (d, J=6.0 Hz, 1H), 4.98-4.96 (m, 1H), 3.80 (br s, 1H), 3.30 (dd, J=4.6, 15.2 Hz, 1H), 3.03-2.82 (m, 3H), 2.39-2.16 (m, 2H), 1.76 (d, J=6.5 Hz, 3H); ES-LCMS m/z 444.1 [M+H]⁺; and the other enantiomer which was purified by preparative HPLC (column: Agela Durashell C18 150*25 5 u; mobile phase: [water (10 mM NH₄HCO₃)−ACN]; B %: 42%-72%, 9 min) again, followed by lyophilization to yield the other enantiomer (19.20 mg, 42.98 μmol, 18.3% yield, 99.3% purity, SFC: Rₜ=4.509, ee=100%, [α]^{30.1}_{D}=−1.514 (CHCl₃, c=0.031 g/100 mL)) as a white solid. ¹H NMR (400 MHz, CDCl₃) δ ppm 9.29 (br s, 1H), 8.49 (br s, 1H), 8.21 (d, J=9.0 Hz, 1H), 7.84 (br s, 1H), 7.43-7.33 (m, 2H), 7.26 (d, J=7.6 Hz, 1H), 7.10 (t, J 7.3 Hz, 1H), 7.04 (d, J=7.1 Hz, 1H), 6.90 (d, J=7.3 Hz, 1H), 5.36-5.33 (m, 1H), 4.93-4.90 (m, 1H), 3.48 (m, 1H), 3.25 (d, J=15.2 Hz, 1H), 2.98-2.79 (m, 3H), 2.34-2.14 (m, 2H), 1.69 (d, J=6.4 Hz, 3H); ES-LCMS m/z 444.1 [M+H]⁺.

### Example 46

### Synthesis of Compound I-43

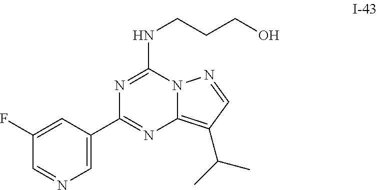 [SMILES: CC(C)c1cnn2c(NCCCO)nc(-c3cncc(F)c3)nc12]

### Synthetic Scheme:

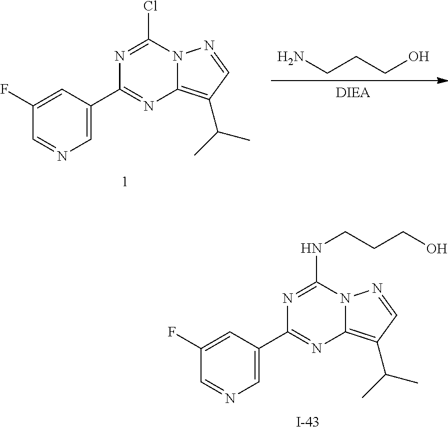

### Step 1: 3-[[2-(5-Fluoro-3-pyridyl)-8-isopropyl-pyrazolo[1,5-a][1,3,5]triazin-4-yl]amino]propan-1-ol (I-43)

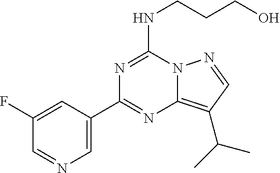 [SMILES: CC(C)c1cnn2c(NCCCO)nc(-c3cncc(F)c3)nc12]

A mixture of 4-chloro-2-(5-fluoro-3-pyridyl)-8-isopropyl-pyrazolo[1,5-a][1,3,5]triazine (50 mg, 161.46 μmol, 1 eq), DIEA (41.73 mg, 322.92 μmol, 56.25 μL, 2 eq) and 3-aminopropan-1-ol (13.34 mg, 177.61 μmol, 13.70 μL, 1.1 eq) in i-PrOH (10 mL) was stirred at 70° C. for 10 h. The mixture was concentrated to yield a residue which was purified on silica gel column chromatography (from PE/EtOAc=5/1 to 1/1 to PE/EtOAc/DCM=1/2/2, TLC: PE/EtOAc=2/1, R_{f}=0.25) to yield 3-[[2-(5-fluoro-3-pyridyl)-8-isopropyl-pyrazolo[1,5-a][1,3,5]triazin-4-yl]amino]propan-1-ol (27.46 mg, 83.12 μmol, 51.5% yield, 100% purity) as a white solid. H NMR (400 MHz, CDCl₃) δ ppm 9.48-9.42 (m, 1H), 8.53 (d, J=2.9 Hz, 1H), 8.42-8.35 (m, 1H), 7.87 (s, 1H), 7.04 (br s, 1H), 3.94 (q, J=6.3 Hz, 2H), 3.83 (q, J=5.5 Hz, 2H), 3.26 (td, J=7.0, 13.9 Hz, 1H), 2.39 (t, J=5.1 Hz, 1H), 2.04-1.94 (m, 2H), 1.38 (d, J=6.8 Hz, 6H); ES-LCMS m/z 331.2 [M+H]⁺.

### Example 47

### Synthesis of Compound I-44a

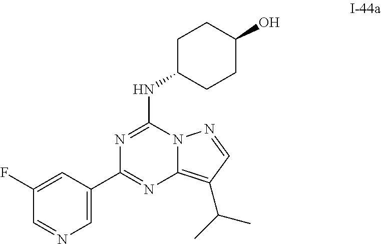 [SMILES: CC(C)c1cnn2c(N[C@H]3CC[C@H](O)CC3)nc(-c3cncc(F)c3)nc12]

### Synthetic Scheme:

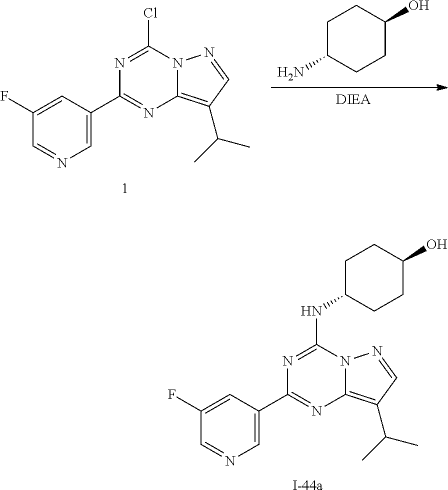

### Step 1: 4-[[2-(5-Fluoro-3-pyridyl)-8-isopropyl-pyrazolo[1,5-a][1,3,5]triazin-4-yl]amino]cyclohexanol (I-44a)

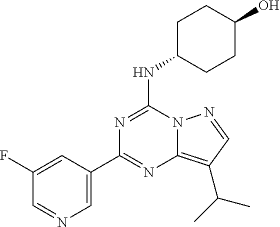 [SMILES: CC(C)c1cnn2c(N[C@H]3CC[C@H](O)CC3)nc(-c3cncc(F)c3)nc12]

To a solution of 4-chloro-2-(5-fluoro-3-pyridyl)-8-isopropyl-pyrazolo[1,5-a][1,3,5]triazine (70 mg, 235.40 μmol, 1 eq) in i-PrOH (10 mL) was added 4-aminocyclohexanol (32.53 mg, 282.49 μmol, 1.2 eq) and DIEA (152.12 mg, 1.18 mmol, 205.02 μL, 5 eq). The mixture was stirred at 70° C. for 12 h. The reaction mixture was concentrated to yield a residue which was purified by preparative HPLC (HCl condition; column: Agela ASB 150*25 mm*5 μm; mobile phase: [water (0.05% HCl)−ACN]; B %: 51%-81%, 8 min), followed by lyophilization to yield 4-[[2-(5-fluoro-3-pyridyl)-8-isopropyl-pyrazolo[1,5-a][1,3,5]triazin-4-yl]amino] cyclohexanol (43.33 mg, 97.73 μmol, 41.5% yield, 100% purity, 2 HCl) as a yellow solid. ¹H NMR (400 MHz, CD3OD) δ ppm 9.46 (s, 1H), 8.80-8.74 (m, 2H), 7.99 (s, 1H), 4.35-4.26 (m, 1H), 3.71-3.62 (m, 1H), 3.30-3.23 (m, 1H), 2.17 (d, J=11.0 Hz, 2H), 2.08 (d, J=11.5 Hz, 2H), 1.72-1.60 (m, 2H), 1.58-1.47 (m, 2H), 1.41 (d, J=6.8 Hz, 6H); ES-LCMS m/z 371.3 [M+H]⁺.

### Example 48

### Synthesis of Compound I-45

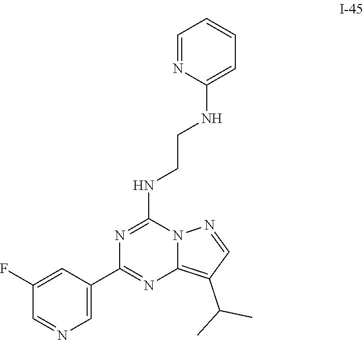 [SMILES: CC(C)c1cnn2c(NCCNc3ccccn3)nc(-c3cncc(F)c3)nc12]

### Synthetic Scheme:

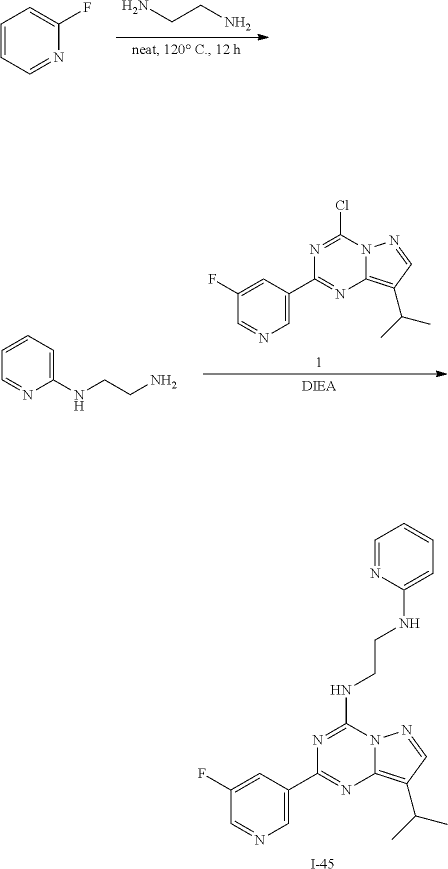

### Step 1: N′-(2-Pyridyl)ethane-1,2-diamine

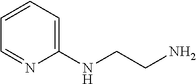

A mixture of 2-fluoropyridine (5 g, 51.50 mmol, 4.42 mL, 1 eq) and ethane-1,2-diamine (2.79 g, 46.35 mmol, 3.10 mL, 0.9 eq) was stirred at 120° C. for 12 h. To the reaction mixture was added EtOAc (50 mL) and stirred at 25° C. for 4 h. The suspension was filtered and solid was collected, washed with EtOAc (10 mL×2), treated under vacuum to yield N′-(2-pyridyl)ethane-1,2-diamine (3.7 g, 2.70 mmol, 5.2% yield, 10% purity) as a white solid. ¹H NMR (400 MHz, DMSO-d₆) δ ppm 7.96 (d, J=4.0 Hz, 1H), 7.35 (t, J=6.9 Hz, 1H), 6.58 (br s, 1H), 6.46-6.43 (m, 1H), 3.34-3.30 (m, 2H), 2.53-2.51 (m, 2H); ES-LCMS m/z 138.1 [M+H]⁺.

### Step 2: N′-[2-(5-Fluoro-3-pyridyl)-8-isopropyl-pyrazolo[1,5-a][1,3,5]triazin-4-yl]-N-(2-pyridyl)ethane-1,2-diamine (I-45)

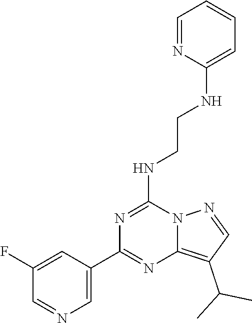 [SMILES: CC(C)c1cnn2c(NCCNc3ccccn3)nc(-c3cncc(F)c3)nc12]

To a solution of N′-(2-pyridyl)ethane-1,2-diamine (483.51 mg, 352.46 μmol, 1.2 eq) and 4-chloro-2-(5-fluoro-3-pyridyl)-8-isopropyl-pyrazolo[1,5-a][1,3,5]triazine (90 mg, 293.72 μmol, 1 eq) in i-PrOH (5 mL) was added DIEA (189.80 mg, 1.47 mmol, 255.80 μL, 5 eq). The mixture was stirred at 70° C. for 12 h. The reaction mixture was concentrated. The residue was purified by preparative HPLC (HCl condition; column: Agela ASB 150*25 mm*5 μm; mobile phase: [water (0.05% HCl)−ACN]; B %: 25%-55%, 8 min), followed by lyophilization to yield N′-[2-(5-fluoro-3-pyridyl)-8-isopropyl-pyrazolo[1,5-a][1,3,5]triazin-4-yl]-N-(2-pyridyl)ethane-1,2-diamine (29.84 mg, 57.87 μmol, 19.7% yield, 97.3% purity, 3 HCl) as a yellow solid. ¹H NMR (400 MHz, CD3OD) δ ppm 9.42 (s, 1H), 8.88 (s, 1H), 8.83-8.78 (m, 1H), 8.04 (s, 1H), 7.86-7.77 (m, 2H), 7.04 (br s, 1H), 6.85 (t, J=6.7 Hz, 1H), 4.11 (t, J=5.6 Hz, 2H), 3.83 (t, J=5.6 Hz, 2H), 3.30-3.23 (m, 1H), 1.40 (d, J=6.8 Hz, 6H); ES-LCMS m/z 393.3 [M+H]⁺.

### Example 49

### Synthesis of Compound I-44b

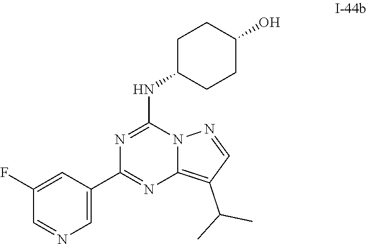 [SMILES: CC(C)c1cnn2c(N[C@H]3CC[C@@H](O)CC3)nc(-c3cncc(F)c3)nc12]

### Synthetic Scheme:

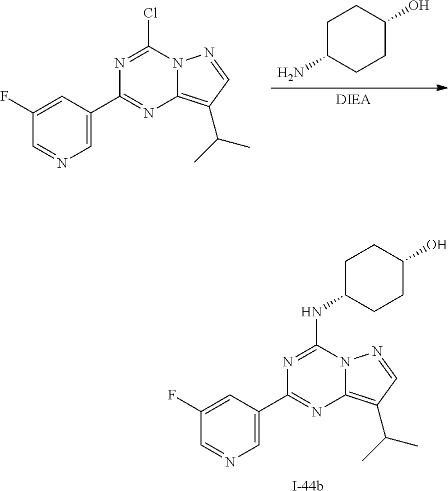

### Step 1: 4-[[2-(5-Fluoro-3-pyridyl)-8-isopropyl-pyrazolo[1,5-a][1,3,5]triazin-4-yl]amino]cyclohexanol (I-44b)

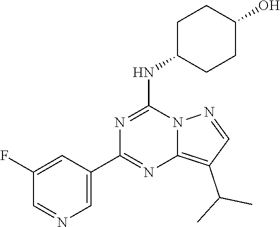 [SMILES: CC(C)c1cnn2c(N[C@H]3CC[C@@H](O)CC3)nc(-c3cncc(F)c3)nc12]

A mixture of 4-chloro-2-(5-fluoro-3-pyridyl)-8-isopropyl-pyrazolo[1,5-a][1,3,5]triazine (60 mg, 195.81 μmol, 1 eq), 4-aminocyclohexanol (22.55 mg, 195.81 μmol, 1 eq) and DIEA (75.92 mg, 587.43 μmol, 102.32 μL, 3 eq) in i-PrOH (2 mL) was stirred at 60° C. for 4 h. The reaction mixture was concentrated under reduced pressure to yield a residue which was purified by preparative HPLC (column: Agela ASB 150*25 mm*5 um; mobile phase: [water (0.05% HCl)−ACN]; B %: 52%-82%, 8 min). The desired fraction was lyophilized to yield 4-[[2-(5-fluoro-3-pyridyl)-8-isopropyl-pyrazolo[1,5-a][1,3,5]triazin-4-yl]amino] cyclohexanol (38.92 mg, 87.79 μmol, 44.8% yield, 100.0% purity, 2HCl) as a yellow solid. ¹H NMR (400 MHz, CD3OD) δ 9.39 (s, 1H), 8.55 (d, J=2.7 Hz, 1H), 8.53-8.47 (m, 1H), 7.96 (s, 1H), 4.39-4.29 (m, 1H), 4.00-3.90 (m, 1H), 3.27-3.22 (m, 1H), 2.04-1.94 (m, 2H), 1.93-1.76 (m, 6H), 1.39 (d, J=7.1 Hz, 6H); ES-LCMS m/z 371.2 [M+H]⁺.

### Example 50

### Synthesis of Compound I-47

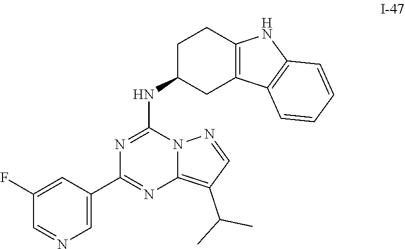 [SMILES: CC(C)c1cnn2c(N[C@H]3CCc4[nH]c5ccccc5c4C3)nc(-c3cncc(F)c3)nc12]

### Synthetic Scheme:

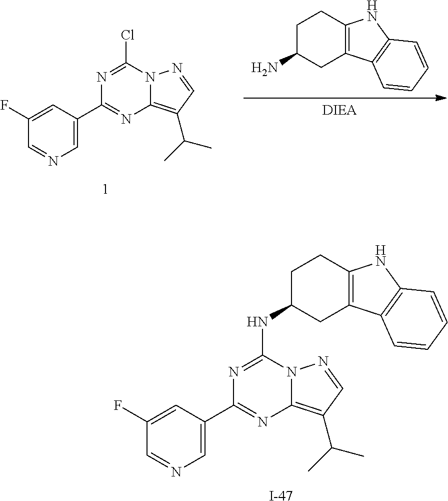

### Step 1: (3S)—N-[2-(5-Fluoro-3-pyridyl)-8-isopropyl-pyrazolo[1,5-a][1,3,5]triazin-4-yl]-2,3,4,9-tetrahydro-1H-carbazol-3-amine (I-47)

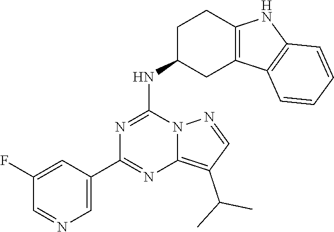 [SMILES: CC(C)c1cnn2c(N[C@H]3CCc4[nH]c5ccccc5c4C3)nc(-c3cncc(F)c3)nc12]

To a solution of 4-chloro-2-(5-fluoro-3-pyridyl)-8-isopropyl-pyrazolo[1,5-a][1,3,5]triazine (150 mg, 484.38 μmol, 1 eq) in i-PrOH (10 mL) was added DIEA (313.02 mg, 2.42 mmol, 421.85 μL, 5 eq) and (3S′)-2,3,4,9-tetrahydro-1H-carbazol-3-amine (94.73 mg, 508.60 μmol, 1.05 eq). The mixture was stirred at 60° C. for 1 h. The reaction mixture was concentrated to yield a residue which was added to MeOH (5 mL) and stirred at 29° C. for 30 min. The mixture was filtered and the filtered cake was dissolved in MeOH (100 mL) and stirred at 70° C. for 15 min. Then the solution was concentrated under vacuum by oil pump at 60° C. for 1 h to yield (3S)—N-[2-(5-fluoro-3-pyridyl)-8-isopropyl-pyrazolo[1,5-a][1,3,5]triazin-4-yl]-2,3,4,9-tetrahydro-1H-carbazol-3-amine (111.58 mg, 252.73 μmol, 52.2% yield, 100.0% purity, [α]^{27.4}_{D}=−17.220 (MeOH, c=0.050 g/100 mL)) as a white solid. ¹H NMR (400 MHz, CDCl₃) δ ppm 9.52 (s, 1H), 8.56 (d, J=2.8 Hz, 1H), 8.49-8.42 (m, 1H), 7.88 (s, 1H), 7.85 (s, 1H), 7.48 (d, J=7.8 Hz, 1H), 7.35 (d, J=8.0 Hz, 1H), 7.22-7.16 (m, 1H), 7.15-7.10 (m, 1H), 6.71 (d, J=8.0 Hz, 1H), 4.97 (m, 1H), 3.45-3.24 (m, 2H), 3.08-2.91 (m, 3H), 2.46-2.27 (m, 2H), 1.41 (d, J=6.8 Hz, 6H); ES-LCMS m/z 442.2 [M+H]⁺.

### Example 51

### Synthesis of Compound I-48a, I-48b and I-48c

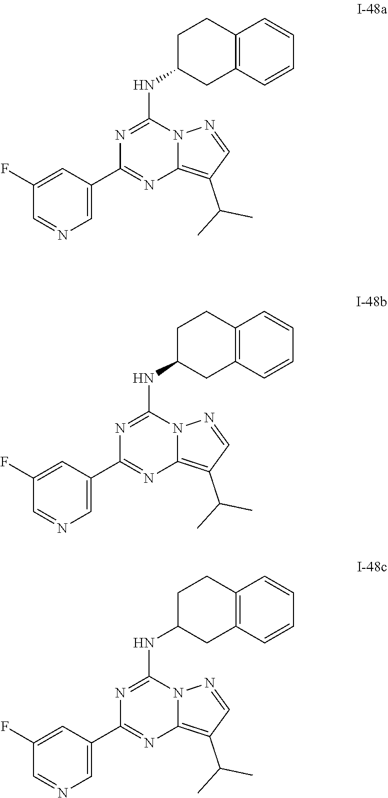 [SMILES: CC(C)c1cnn2c(N[C@@H]3CCc4ccccc4C3)nc(-c3cncc(F)c3)nc12 ; CC(C)c1cnn2c(N[C@H]3CCc4ccccc4C3)nc(-c3cncc(F)c3)nc12 ; CC(C)c1cnn2c(NC3CCc4ccccc4C3)nc(-c3cncc(F)c3)nc12]

### Synthetic Scheme:

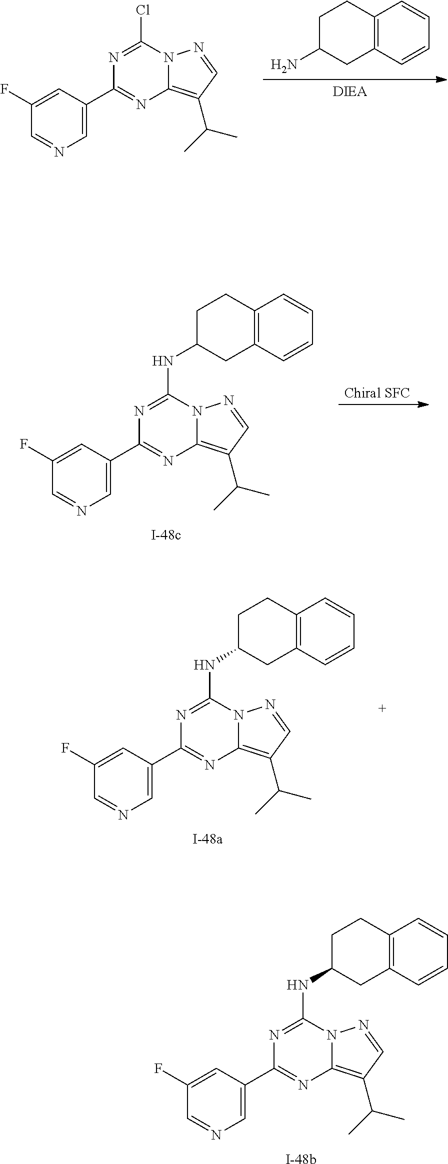

### Step 1: 2-(5-Fluoro-3-pyridyl)-8-isopropyl-N-[(2R)-tetralin-2-yl]pyrazolo[1,5-a][1,3,5]triazin-4-amine (I-48a) & 2-(5-Fluoro-3-pyridyl)-8-isopropyl-N-[(2S)-tetralin-2-yl]pyrazolo[1,5-a][1,3,5]triazin-4-amine (I-48b)

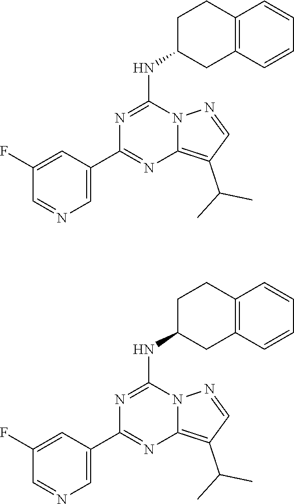 [SMILES: CC(C)c1cnn2c(N[C@@H]3CCc4ccccc4C3)nc(-c3cncc(F)c3)nc12 ; CC(C)c1cnn2c(N[C@H]3CCc4ccccc4C3)nc(-c3cncc(F)c3)nc12]

A mixture of 4-chloro-2-(5-fluoro-3-pyridyl)-8-isopropyl-pyrazolo[1,5-a][1,3,5]triazine (150 mg, 484.38 μmol, 1 eq), tetralin-2-amine (88.97 mg, 484.38 μmol, 1 eq, HCl) and DIEA (187.81 mg, 1.45 mmol, 253.11 μL, 3 eq) in i-PrOH (10 mL) was stirred at 60° C. for 12 h. The reaction mixture was cooled to 25° C. and filtered. The solid was washed with i-PrOH (10 mL) and dried under reduced pressure. The residue was separated by chiral SFC (column: DAICEL CHIRALPAK AD-H (250 mm*30 mm, 5 um); mobile phase: [0.1% NH₃H₂O IPA]; B %: 30%-30%) to yield peak 1 and peak 2. One of these peaks was concentrated under reduced pressure to yield a residue which was lyophilized to yield an enantiomer (49.73 mg, 123.56 μmol, 25.5% yield, 100.0% purity, SFC: Rₜ=4.600, ee=99.758%, [α]^{26.6}_{D}=+13.824 (CHCl₃, c=0.106 g/100 mL)) as a white solid. ¹H NMR (400 MHz, CDCl₃) δ ppm 9.50 (s, 1H), 8.55 (d, J=2.8 Hz, 1H), 8.47-8.39 (m, 1H), 7.87 (s, 1H), 7.21-7.12 (m, 4H), 6.62 (d, J=7.6 Hz, 1H), 4.85-4.73 (m, 1H), 3.40 (dd, J=5.2, 16.4 Hz, 1H), 3.34-3.24 (m, 1H), 3.14-3.02 (m, 2H), 2.98 (dd, J=8.4, 16.4 Hz, 1H), 2.40-2.31 (m, 1H), 2.08 (dtd, J=6.4, 9.2, 12.8 Hz, 1H), 1.42 (d, J=7.2 Hz, 6H); ES-LCMS m/z 403.2 [M+H]⁺. The other of these peaks was concentrated under reduced pressure to yield a residue which was lyophilized to yield the other enantiomer (49.48 mg, 122.94 μmol, 25.4% yield, 100.0% purity, SFC: Rₜ=4.931, ee=98.946%, [α]^{26.6}_{D}=−18.677 (CHCl₃, c=0.110 g/100 mL)) as a white solid. ¹H NMR (400 MHz, CDCl₃) δ ppm 9.53-9.47 (m, 1H), 8.55 (d, J=2.8 Hz, 1H), 8.49-8.38 (m, 1H), 7.87 (s, 1H), 7.20-7.12 (m, 4H), 6.62 (d, J=7.6 Hz, 1H), 4.87-4.73 (m, 1H), 3.39 (dd, J=5.2, 16.0 Hz, 1H), 3.30 (td, J=7.2, 13.6 Hz, 1H), 3.13-3.02 (m, 2H), 2.98 (dd, J=8.4, 16.4 Hz, 1H), 2.40-2.31 (m, 1H), 2.13-2.01 (m, 1H), 1.42 (d, J=7.2 Hz, 6H); ES-LCMS m z 403.2 [M+H]⁺.

### Example 52

### Synthesis of Compound I-49

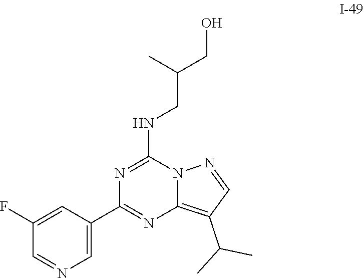 [SMILES: CC(CO)CNc1nc(-c2cncc(F)c2)nc2c(C(C)C)cnn12]

### Synthetic Scheme:

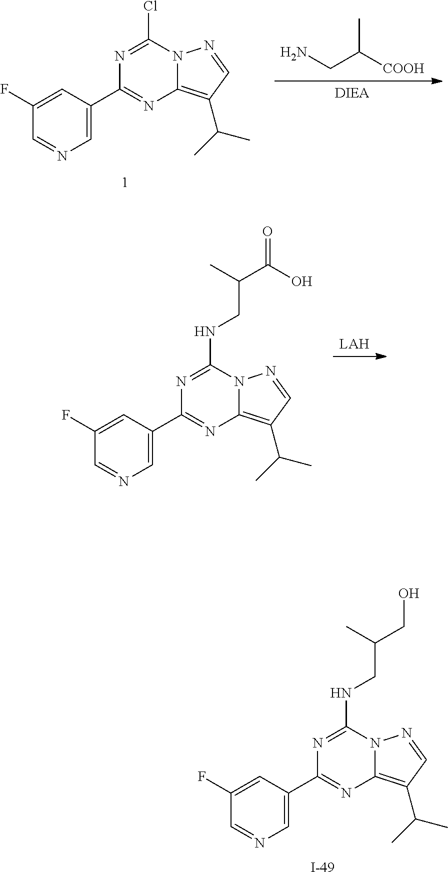

### Step 1: 3-[[2-(5-Fluoro-3-pyridyl)-8-isopropyl-pyrazolo[1,5-a][1,3,5]triazin-4-yl]amino]-2-methyl-propanoic acid

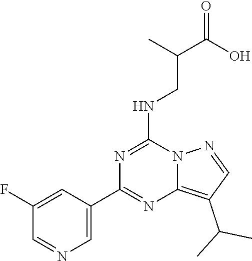 [SMILES: CC(CNc1nc(-c2cncc(F)c2)nc2c(C(C)C)cnn12)C(=O)O]

To a solution of 4-chloro-2-(5-fluoro-3-pyridyl)-8-isopropyl-pyrazolo[1,5-a][1,3,5]triazine (200 mg, 658.87 μmol, 1 eq) in i-PrOH (5 mL) was added DIEA (85.15 mg, 658.87 μmol, 114.76 μL, 1 eq) and 3-amino-2-methyl-propanoic acid (71.34 mg, 691.82 μmol, 1.05 eq). The mixture was stirred at 60° C. for 1 h. The reaction mixture was concentrated to yield 3-[[2-(5-fluoro-3-pyridyl)-8-isopropyl-pyrazolo[1,5-a][1,3,5]triazin-4-yl]amino]-2-methyl-propanoic acid (240 mg, 470.80 μmol, 71.5% yield, 70.3% purity) as a yellow solid which was used in the next step without further purification. ¹H NMR (400 MHz, DMSO-d₆) δ ppm 9.38 (t, J=1.4 Hz, 1H), 8.96 (s, 1H), 8.72 (d, J=2.8 Hz, 1H), 8.45-8.39 (m, 1H), 8.12 (s, 1H), 3.56 (m, 1H), 3.20 (m, 1H), 3.08 (d, J=7.3 Hz, 1H), 2.96-2.90 (m, 1H), 1.35 (d, J=6.8 Hz, 6H), 1.15 (d, J=7.3 Hz, 3H); ES-LCMS m/z 444.2 [M+H]⁺.

### Step 2: 3-[[2-(5-Fluoro-3-pyridyl)-8-isopropyl-pyrazolo[1,5-a][1,3,5]triazin-4-yl]amino]-2-methyl-propan-1-ol (I-49)

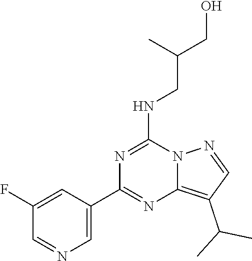 [SMILES: CC(CO)CNc1nc(-c2cncc(F)c2)nc2c(C(C)C)cnn12]

To a solution of 3-[[2-(5-fluoro-3-pyridyl)-8-isopropyl-pyrazolo[1,5-a][1,3,5]triazin-4-yl] amino]-2-methyl-propanoic acid (240 mg, 470.80 μmol, 1 eq) in THE (10 mL) was cooled to 0° C. and added LAH (89.33 mg, 2.35 mmol, 5 eq) under ice-water bath. The mixture was stirred at 0° C. for 2 h under ice-water bath. Water (0.5 mL) and 10% NaOH solution (0.5 mL) was added to the reaction mixture. Then water (0.5 mL) was added to the above mixture and stirred for 15 min. The mixture was filtered and the filtered cake was washed with THE (10 mL×2). The combined organic layers were concentrated to yield a residue which was purified by preparative HPLC (HCl condition; column: Agela ASB 150*25 mm*5 μm; mobile phase: [water (0.05% HCl)−ACN]; B %: 52%-82%, 8 min). The desired fraction was lyophilized to yield 3-[[2-(5-fluoro-3-pyridyl)-8-isopropyl-pyrazolo[1,5-a][1,3,5]triazin-4-yl]amino]-2-methyl-propan-1-ol (61.3 mg, 146.89 μmol, 31.2% yield, 100.0% purity, 2HCl) as a yellow solid. ¹H NMR (400 MHz, CD3OD) δ ppm 9.49-9.41 (m, 1H), 8.70-8.62 (m, 2H), 7.98 (s, 1H), 3.83-3.65 (m, 2H), 3.64-3.54 (m, 2H), 3.30-3.24 (m, 1H), 2.16 (d, J=6.4, 12.8 Hz, 1H), 1.41 (d, J=7.0 Hz, 6H), 1.06 (d, J=7.0 Hz, 3H); ES-LCMS m/z 345.2 [M+H]⁺.

### Example 53

### Synthesis of Compound I-50

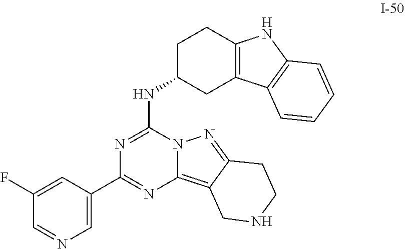 [SMILES: Fc1cncc(-c2nc(N[C@@H]3CCc4[nH]c5ccccc5c4C3)n3nc4c(c3n2)CNCC4)c1]

### Synthetic Scheme:

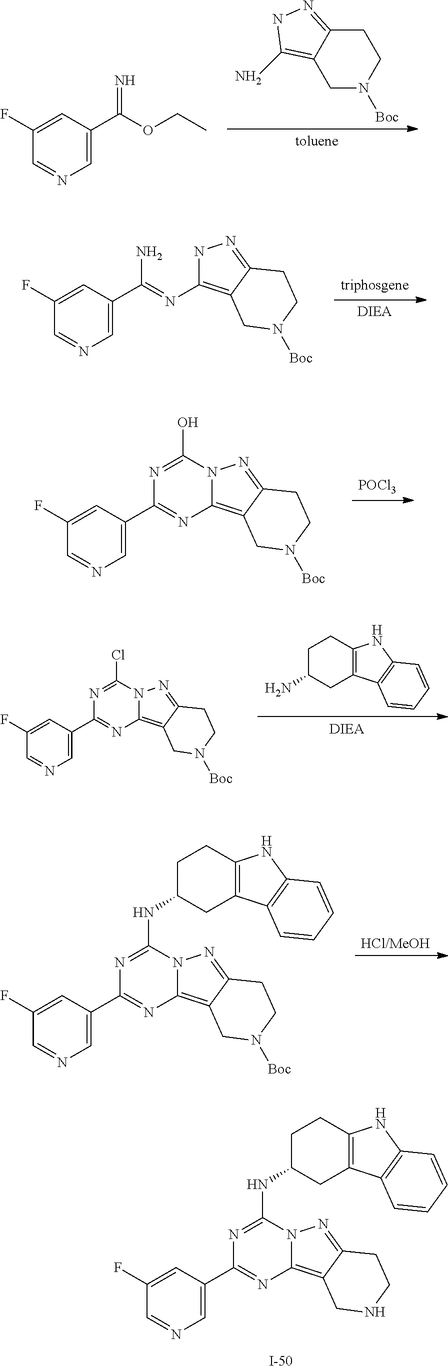

### Step 1: tert-Butyl 3-[(Z)-[amino-(5-fluoro-3-pyridyl)methylene]amino]-2,4,6,7-tetra hydropyrazolo[4,3-c]pyridine-5-carboxylate

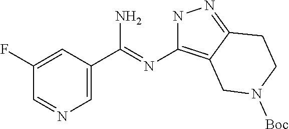 [SMILES: CC(C)(C)OC(=O)N1CCc2n[nH]c(/N=C(\N)c3cncc(F)c3)c2C1]

To a solution of ethyl 5-fluoropyridine-3-carboximidate (742.88 mg, 4.20 mmol, 1 eq) in toluene (20 mL) was added tert-butyl 3-amino-2,4,6,7-tetrahydropyrazolo[4,3-c] pyridine-5-carboxylate (1 g, 4.20 mmol, 1 eq). The mixture was stirred at 110° C. for 12 h. The mixture was concentrated to yield a residue. To the residue was added PE/EtOAc (5/1, 50 mL), stirred at 25° C. for 1 h. The slurry was filtered, the cake was rinsed with PE (30 mL×2), collected and dried in vacuo to yield tert-butyl 3-[(Z)-[amino-(5-fluoro-3-pyridyl) methylene]amino]-2,4,6,7-tetrahydropyrazolo[4,3-c]pyridine-5-carboxylate (1.3 g, 3.51 mmol, 83.6% yield, 97.3% purity) as a white solid which was used in the next step without further purification. ¹H NMR (400 MHz, DMSO-d₆) δ ppm 12.20 (s, 1H), 9.04 (br s, 1H), 8.67 (d, J=2.7 Hz, 1H), 8.18 (d, J=9.0 Hz, 1H), 4.35 (s, 2H), 3.61 (t, J=5.7 Hz, 2H), 2.78-2.57 (m, 2H), 1.43 (s, 9H); ES-LCMS m/z 361.2 [M+H]⁺.

### Step 2: tert-Butyl 2-(5-fluoro-3-pyridyl)-4-hydroxy-8,10-dihydro-7H-pyrido[2,3]pyrazolo [2,4-c][1,3,5]triazine-9-carboxylate

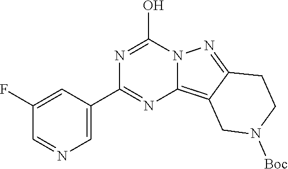 [SMILES: CC(C)(C)OC(=O)N1CCc2nn3c(O)nc(-c4cncc(F)c4)nc3c2C1]

To a solution of tert-butyl 3-[(Z)-[amino-(5-fluoro-3-pyridyl)methylene]amino]-2,4,6,7-tetrahydropyrazolo[4,3-c]pyridine-5-carboxylate (600 mg, 1.62 mmol, 1 eq) in THE (15 mL) and 1,4-dioxane (15 mL) was added triphosgene (480.71 mg, 1.62 mmol, 1 eq) and DIEA (418.72 mg, 3.24 mmol, 564.31 μL, 2 eq). The mixture was stirred at 80° C. for 2 h. The reaction mixture was concentrated under reduced pressure to yield a residue which was purified by flash silica gel chromatography (DCM/MeOH=10/1, DCM/MeOH=10/1, R_{f}=0.29) to yield tert-butyl 2-(5-fluoro-3-pyridyl)-4-hydroxy-8,10-dihydro-7H-pyrido[2,3]pyrazolo[2,4-c][1,3,5]triazine-9-carboxylate (180 mg, 335.42 μmol, 20.7% yield, 72% purity) as a yellow solid. ¹H NMR (400 MHz, CDCl₃) δ ppm 9.25 (br s, 1H), 8.66-8.43 (m, 1H), 8.33 (br s, 1H), 4.68 (m, 2H), 3.78 (m, 2H), 3.02-2.77 (m, 2H), 1.51 (br s, 9H); ES-LCMS m/z 387.2 [M+H]⁺.

### Step 3: 4-Chloro-2-(5-fluoro-3-pyridyl)-8,10-dihydro-7H-pyrido[2,3]pyrazolo[2,4-c][1,3,5]triazine-9-carboxylate

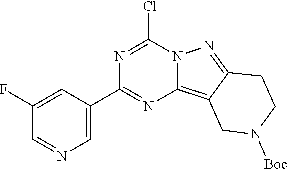 [SMILES: CC(C)(C)OC(=O)N1CCc2nn3c(Cl)nc(-c4cncc(F)c4)nc3c2C1]

To a solution of tert-butyl 2-(5-fluoro-3-pyridyl)-4-hydroxy-8,10-dihydro-7H-pyrido[2,3] pyrazolo[2,4-c][1,3,5]triazine-9-carboxylate (150 mg, 279.52 μmol, 1 eq) in toluene (15 mL) was added POCl₃ (428.58 mg, 2.80 mmol, 259.75 μL, 10 eq) and DIEA (361.26 mg, 2.80 mmol, 486.87 μL, 10 eq). The mixture was stirred at 130° C. for 2 h. The reaction mixture was cooled to 0° C., diluted with EtOAc (100 mL), quenched with cold 18% aq. K₂HPO₄. 3H₂O (20 mL). The solution was separated and extracted with EtOAc (30 mL×3). The organic layer was washed with brine (50 mL), dried over Na₂SO₄, filtered, concentrated under reduced pressure to yield tert-butyl 4-chloro-2-(5-fluoro-3-pyridyl)-8,10-dihydro-7H-pyrido[2,3]pyrazolo[2,4-c][1,3,5]triazine-9-carboxylate (113 mg, crude) as a yellow solid which was used in the next step without further purification. ¹H NMR (400 MHz, CDCl₃) δ ppm 9.49 (s, 1H), 8.63 (d, J=2.8 Hz, 1H), 8.44 (d, J=9.3 Hz, 1H), 4.80 (m, 2H), 3.86 (m, 2H), 3.07-3.04 (m, 2H), 1.52 (s, 9H); ES-LCMS m/z 405.1, 407.1 [M+H]⁺.

### Step 4: 2-(5-Fluoro-3-pyridyl)-4-[[(3R)-2,3,4,9-tetrahydro-1H-carbazol-3-yl]amino]-8,10-dihydro-7H-pyrido[2,3]pyrazolo[2,4-c][1,3,5]triazine-9-carboxylate

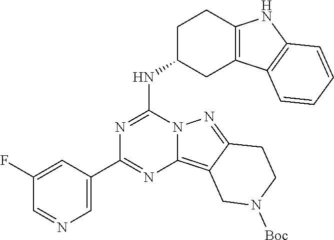 [SMILES: CC(C)(C)OC(=O)N1CCc2nn3c(N[C@@H]4CCc5[nH]c6ccccc6c5C4)nc(-c4cncc(F)c4)nc3c2C1]

To a solution of tert-butyl 4-chloro-2-(5-fluoro-3-pyridyl)-8,10-dihydro-7H-pyrido[2,3] pyrazolo[2,4-c][1,3,5]triazine-9-carboxylate (113 mg, 279.13 μmol, 1 eq) in i-PrOH (10 mL) was added DIEA (108.23 mg, 837.40 μmol, 145.86 μL, 3 eq) and (3R)-2,3,4,9-tetrahydro-1H-carbazol-3-amine (51.99 mg, 279.13 μmol, 1 eq). The mixture was stirred at 60° C. for 2 h. TLC (PE/EtOAc=2/1, R_{f}=0.17) showed the starting material was consumed and one new spot formed. The reaction mixture was concentrated under reduced pressure to yield a residue which was purified by flash silica gel chromatography (from PE/EtOAc=1/0 to 1/1, TLC: PE/EtOAc=3/1, R_{f}=0.17) to yield tert-butyl 2-(5-fluoro-3-pyridyl)-4-[[(3R)-2,3,4,9-tetrahydro-1H-carbazol-3-yl]amino]-8,10-dihydro-7H-pyrido[2,3]pyrazolo[2,4-c][1,3,5]triazine-9-carboxylate (80 mg, 124.05 μmol, 44.4% yield, 86% purity) as a yellow solid. ¹H NMR (400 MHz, CDCl₃) δ ppm 9.49 (s, 1H), 8.57 (d, J=2.8 Hz, 1H), 8.44 (d, J=9.3 Hz, 1H), 7.94 (s, 1H), 7.47 (d, J=7.8 Hz, 1H), 7.35 (d, J=8.0 Hz, 1H), 7.22-7.09 (m, 2H), 6.67 (d, J=7.0 Hz, 1H), 5.04-4.85 (m, 1H), 4.73 (br s, 2H), 3.81 (br s, 2H), 3.37 (dd, J=5.1, 15.4 Hz, 1H), 3.15-2.82 (m, 5H), 2.47-2.23 (m, 2H), 1.52 (s, 9H); ES-LCMS m/z 555.2 [M+H]⁺.

### Step 5: 2-(5-Fluoro-3-pyridyl)-N-[(3R)-2,3,4,9-tetrahydro-1H-carbazol-3-yl]-7,8,9,10-tetrahydropyrido[2,3]pyrazolo[2,4-c][1,3,5]triazin-4-amine (I-50)

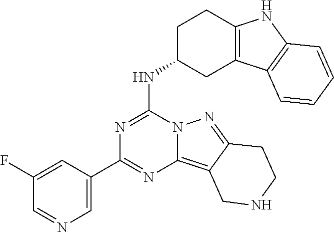 [SMILES: Fc1cncc(-c2nc(N[C@@H]3CCc4[nH]c5ccccc5c4C3)n3nc4c(c3n2)CNCC4)c1]

To a solution of tert-butyl 2-(5-fluoro-3-pyridyl)-4-[[(3R)-2,3,4,9-tetrahydro-1H-carbazol-3-yl] amino]-8,10-dihydro-7H-pyrido[2,3]pyrazolo[2,4-c][1,3,5]triazine-9-carboxylate (75 mg, 116.30 μmol, 1 eq) in DCM (10 mL) was added HCl/MeOH (4 M, 3 mL, 103.18 eq). The mixture was stirred at 25° C. for 1 h. The reaction mixture was concentrated under reduced pressure to yield a residue. To the residue was added DCM (10 mL), stirred at 25° C. for 0.5 h. The slurry was filtered, the filter cake was rinsed with DCM (5 mL×2). The solid was dissolved in MeCN (10 mL) and H₂O (10 mL), followed by lyophilization to yield 2-(5-fluoro-3-pyridyl)-N-[(3R)-2,3,4,9-tetrahydro-1H-carbazol-3-yl]-7,8,9,10-tetrahydropyrido[2,3]pyrazolo[2,4-c][1,3,5]triazin-4-amine (53.32 mg, 94.56 μmol, 81.3% yield, 100% purity, 3HCl, [α]^{22.1}_{D}=+15.441 (DMSO, c=0.109 g/100 mL) as a yellow solid. ¹H NMR (400 MHz, CD3OD) δ ppm 9.41 (s, 1H), 8.65-8.59 (m, 2H), 7.35 (d, J=7.8 Hz, 1H), 7.26 (d, J=7.8 Hz, 1H), 7.06-7.00 (m, 1H), 6.97-6.92 (m, 1H), 4.96-4.91 (m, 1H), 4.51 (s, 2H), 3.66 (t, J=6.2 Hz, 2H), 3.28-3.20 (m, 3H), 3.13-2.87 (m, 3H), 2.42-2.20 (m, 2H); ES-LCMS m/z 455.2 [M+H]⁺.

### Example 54

### Synthesis of Compound I-51

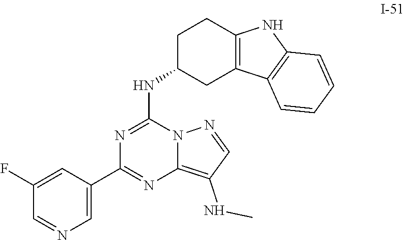 [SMILES: CNc1cnn2c(N[C@@H]3CCc4[nH]c5ccccc5c4C3)nc(-c3cncc(F)c3)nc12]

### Synthetic Scheme:

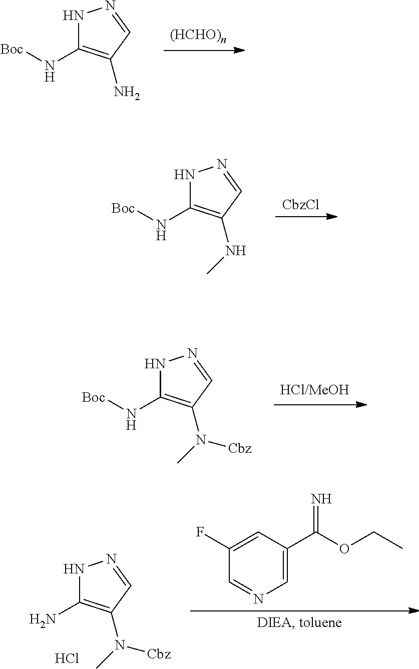

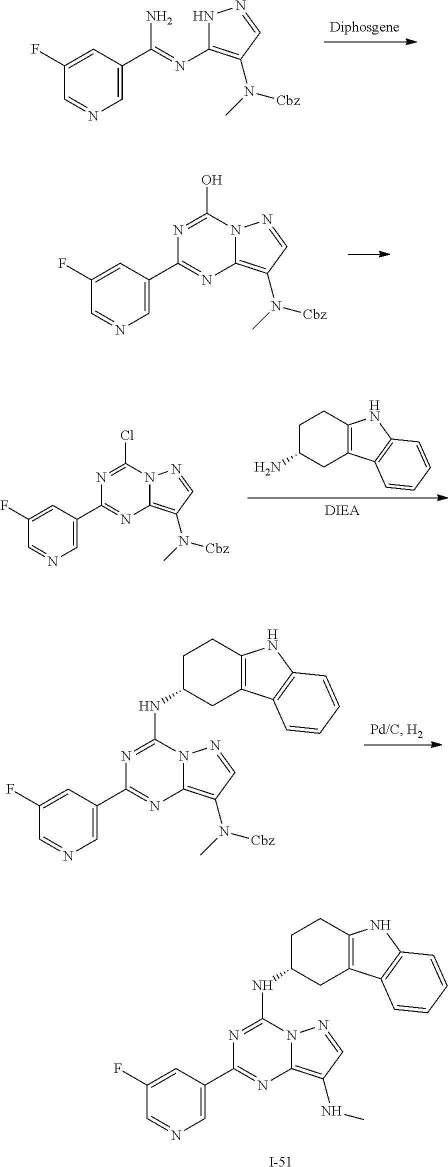

### Step 1: tert-Butyl N-[4-(methylamino)-1H-pyrazol-5-yl]carbamate

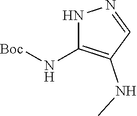

To a solution of tert-butyl N-(4-amino-1H-pyrazol-5-yl)carbamate (6 g, 28.76 mmol, 1 eq) in MeOH (60 mL) was added a solution of HCHO (2.33 g, 28.76 mmol, 2.14 mL, 37% purity, 1 eq). Then mixture was stirred at 50° C. for 11 h. Then NaBH₃CN (5.42 g, 86.27 mmol, 3 eq) was added mixture. Then mixture was stirred at 50° C. for 1 h. The reaction mixture was concentrated under reduced pressure to yield a residue which was added sat. aq. NaHCO₃ (100 mL) and EtOAc (100 mL). Then the mixture was extracted with EtOAc (100 mL×3), dried over Na₂SO₄, filtered and concentrated under reduced pressure to yield a residue which was purified on silica gel column chromatography (from pure PE to PE/EtOAc=1/1, TLC: PE/EtOAc=1/1, R_{f}=0.2) to yield tert-butyl N-[4-(methylamino)-1H-pyrazol-5-yl]carbamate (2.6 g, 8.57 mmol, 29.8% yield, 70.0% purity) as a yellow solid. ¹H NMR (400 MHz, CDCl₃) δ ppm 7.35 (s, 1H), 6.96 (s, 1H), 2.71 (s, 3H), 1.45 (s, 9H); ES-LCMS m/z 157.2 [M−t-Bu+H]⁺.

### Step 2: Benzyl N-[5-(tert-butoxycarbonylamino)-1H-pyrazol-4-yl]-N-methyl-carbamate

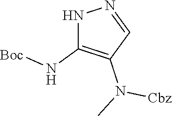 [SMILES: CN(C(=O)OCc1ccccc1)c1cn[nH]c1NC(=O)OC(C)(C)C]

To a solution of tert-butyl N-[4-(methylamino)-1H-pyrazol-5-yl]carbamate (2.6 g, 8.57 mmol, 1 eq) in DCM (70 mL) was added CbzCl (1.76 g, 10.29 mmol, 1.46 mL, 1.2 eq) and Et₃N (2.60 g, 25.72 mmol, 3.58 mL, 3 eq). Then the mixture was stirred at 30° C. for 1 h. The reaction mixture was concentrated under reduced pressure to yield a residue which was added sat. aq. NaHCO₃ (30 mL) and EtOAc (30 mL). Then the mixture was extracted with EtOAc (30 mL×3), dried over Na₂SO₄, filtered and concentrated to yield a residue which was purified on silica gel column chromatography (from PE/EtOAc=1/0 to 1/1, TLC: PE/EtOAc=1/1, R_{f}=0.5) to yield benzyl N-[5-(tert-butoxycarbonylamino)-1H-pyrazol-4-yl]-N-methyl-carbamate (1.6 g, 4.16 mmol, 48.5% yield, 90.0% purity) as yellow oil. ¹H NMR (400 MHz, CDCl₃) δ ppm 7.12-7.44 (m, 6H), 4.99-5.31 (m, 2H), 3.24 (s, 3H), 1.39-1.55 (m, 9H); ES-LCMS m/z 291.1 [M−t-Bu+H]⁺.

### Step 3: Benzyl N-(5-amino-1H-pyrazol-4-yl)-N-methyl-carbamate

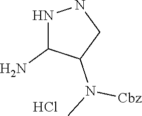 [SMILES: CN(C(=O)OCc1ccccc1)C1CNNC1N ; Cl]

To a solution of benzyl N-[5-(tert-butoxycarbonylamino)-1H-pyrazol-4-yl]-N-methyl-carbamate (1.6 g, 4.16 mmol, 1 eq) in DCM (27 mL) was added HCl/MeOH (4 M, 9 mL, 8.66 eq). Then mixture was stirred at 30° C. for 1 h. The reaction mixture was concentrated to yield benzyl N-(5-amino-1H-pyrazol-4-yl)-N-methyl-carbamate (1.1 g, 3.89 mmol, 93.6% yield, crude, HCl) as yellow oil which was used in next step without further purification. ¹H NMR (400 MHz, CDCl₃) δ ppm 7.48 (s, 1H), 7.22-7.36 (m, 5H), 5.03-5.17 (m, 2H), 3.02-3.26 (m, 3H); ES-LCMS m/z 247.2 [M+H]⁺.

### Step 4: Benzyl N-[5-[(Z)-[amino-(5-fluoro-3-pyridyl)methylene]amino]-1H-pyrazol-4-yl]-N-methyl-carbamate

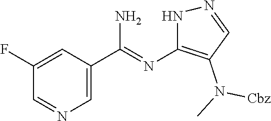 [SMILES: CN(C(=O)OCc1ccccc1)c1cn[nH]c1/N=C(\N)c1cncc(F)c1]

To a solution of benzyl N-(5-amino-1H-pyrazol-4-yl)-N-methyl-carbamate (1.1 g, 4.47 mmol, 1 eq) in toluene (20 mL) was added ethyl 5-fluoropyridine-3-carboximidate (790.70 mg, 4.47 mmol, 1 eq). The mixture was stirred at 120° C. for 12 h. The reaction mixture was concentrated under reduced to yield a residue which was purified on silica gel column chromatography (from pure PE to PE/EtOAc=10/1, TLC: PE/EtOAc=1/10, R_{f}=0.5) to yield benzyl N-[5-[(Z)-[amino-(5-fluoro-3-pyridyl)methylene]amino]-1H-pyrazol-4-yl]-N-methyl-carbamate (730 mg, 1.88 mmol, 48.4% yield, 95.0% purity) as yellow oil. ES-LCMS m/z 369.2 [M+H]⁺.

### Step 5: Benzyl N-[2-(5-fluoro-3-pyridyl)-4-hydroxy-pyrazolo[1,5-a][1,3,5]triazin-8-yl]-N-methyl-carbamate

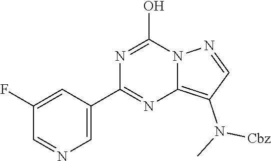 [SMILES: CN(C(=O)OCc1ccccc1)c1cnn2c(O)nc(-c3cncc(F)c3)nc12]

To a solution of benzyl N-[5-[(Z)-[amino-(5-fluoro-3-pyridyl)methylene]amino]-1H-pyrazol-4-yl]-N-methyl-carbamate (300 mg, 732.97 μmol, 1 eq) in 1,4-dioxane (15 mL) and THE (15 mL) was added diphosgene (435.02 mg, 2.20 mmol, 265.25 μL, 3 eq). The mixture was stirred at 80° C. for 12 h. After filtration, the filter cake was washed with PE/EtOAc (2/1, 5 mL×2) and dried in vacuo to yield benzyl N-[2-(5-fluoro-3-pyridyl)-4-hydroxy-pyrazolo[1,5-a][1,3,5]triazin-8-yl]-N-methyl-carbamate (200 mg, 405.72 μmol, 55.4% yield, 80% purity) as a yellow solid. ¹H NMR (400 MHz, DMSO-d₆) δ ppm 9.04 (s, 1H), 8.77 (d, J=2.7 Hz, 1H), 8.25 (d, J=7.8 Hz, 1H), 8.17 (br s, 1H), 7.24 (br s, 5H), 5.07 (br s, 2H), 3.45-3.38 (m, 3H); ES-LCMS m/z 395.2 [M+H]⁺.

### Step 6: Benzyl N-[4-chloro-2-(5-fluoro-3-pyridyl)pyrazolo[1,5-a][1,3,5]triazin-8-yl]-N-methyl-carbamate

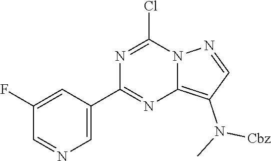 [SMILES: CN(C(=O)OCc1ccccc1)c1cnn2c(Cl)nc(-c3cncc(F)c3)nc12]

To a mixture of benzyl N-[2-(5-fluoro-3-pyridyl)-4-hydroxy-pyrazolo[1,5-a][1,3,5]triazin-8-yl]-N-methyl-carbamate (200 mg, 405.72 μmol, 1 eq) and DIEA (262.18 mg, 2.03 mmol, 353.35 μL, 5 eq) in toluene (6 mL) was added POCl₃ (3.6 g, 23.48 mmol, 2.18 mL, 57.87 eq). The mixture was stirred at 130° C. for 3 h. The mixture was concentrated to yield benzyl N-[4-chloro-2-(5-fluoro-3-pyridyl)pyrazolo[1,5-a][1,3,5]triazin-8-yl]-N-methyl-carbamate (150 mg, 247.05 μmol, 60.9% yield, 80% purity, 2 HCl) as a yellow solid. ¹H NMR (400 MHz, CDCl₃) δ ppm 9.49 (br s, 1H), 8.69 (m, 2H), 7.37-7.22 (m, 5H), 7.13-7.11 (m, 1H), 5.26-5.07 (m, 2H), 3.54-3.43 (m, 3H); ES-LCMS m/z 413.1 [M+H]⁺.

### Step 7: Benzyl N-[2-(5-fluoro-3-pyridyl)-4-[[(3R)-2,3,4,9-tetrahydro-1H-carbazol-3-yl]amino]pyrazolo[1,5-a][1,3,5]triazin-8-yl]-N-methyl-carbamate

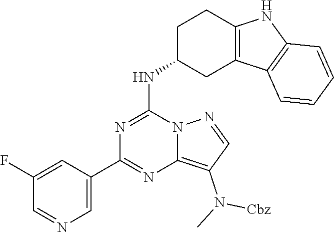 [SMILES: CN(C(=O)OCc1ccccc1)c1cnn2c(N[C@@H]3CCc4[nH]c5ccccc5c4C3)nc(-c3cncc(F)c3)nc12]

A mixture of benzyl N-[4-chloro-2-(5-fluoro-3-pyridyl)pyrazolo[1,5-a][1,3,5]triazin-8-yl]-N -methyl-carbamate (140 mg, 230.58 μmol, 1 eq, 2 HCl), DIEA (149.01 mg, 1.15 mmol, 200.82 μL, 5 eq) and (3R)-2,3,4,9-tetrahydro-1H-carbazol-3-amine (42.95 mg, 230.58 μmol, 1 eq) in CH₃CN (10 mL) was stirred at 70° C. for 2 h. The mixture was concentrated to yield a residue was purified with preparative TLC (PE/EtOAc=3/2, R_{f}=0.5) to yield benzyl N-[2-(5-fluoro-3-pyridyl)-4-[[(3R)-2,3,4,9-tetrahydro-1H-carbazol-3-yl]amino]pyrazolo[1,5-a][1,3,5]triazin-8-yl]-N-methyl-carbamate (120 mg, 202.63 μmol, 87.88% yield, 95% purity) as a yellow solid. ¹H NMR (400 MHz, CDCl₃) δ ppm 9.41 (s, 1H), 8.50 (br s, 1H), 8.33 (d, J=9.5 Hz, 1H), 7.82 (s, 1H), 7.40 (d, J=7.3 Hz, 3H), 7.27 (d, J=7.8 Hz, 3H), 7.14-7.01 (m, 2H), 6.72 (br s, 1H), 5.23-5.06 (m, 2H), 4.89 (m, 1H), 3.52-3.38 (m, 3H), 3.33-3.23 (m, 1H), 2.97-2.85 (m, 3H), 2.34-2.21 (m, 2H); ES-LCMS m/z 563.3 [M+H]⁺.

### Step 8: 2-(5-Fluoro-3-pyridyl)-N₈-methyl-N₄-[(3R)-2,3,4,9-tetrahydro-1H-carbazol-3-yl]pyrazolo[1,5-a][1,3,5]triazine-4,8-diamine (I-51)

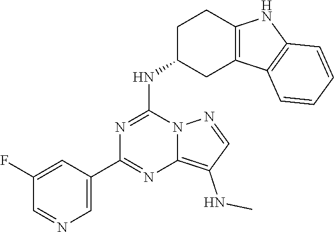 [SMILES: CNc1cnn2c(N[C@@H]3CCc4[nH]c5ccccc5c4C3)nc(-c3cncc(F)c3)nc12]

To a solution of benzyl N-[2-(5-fluoro-3-pyridyl)-4-[[(3R)-2,3,4,9-tetrahydro-1H-carbazol-3-yl]amino]pyrazolo[1,5-a][1,3,5]triazin-8-yl]-N-methyl-carbamate (110 mg, 185.75 μmol, 1 eq) in MeOH (8 mL) and THE (8 mL) was added Pd/C (50 mg, 185.75 μmol, 10% purity, 1.00 eq) under N₂ atmosphere. The mixture was stirred under H₂ atmosphere (15 psi) at 30° C. for 3 h. After filtration, the filtrate was concentrated to yield a residue which was purified by preparative HPLC (column: Agela ASB 150*25 mm*5 μm; mobile phase: [water (0.05% HCl)−ACN]; B %: 36%-66%, 8 min), followed by lyophilization to yield 2-(5-fluoro-3-pyridyl)-N8-methyl-N4-[(3R)-2,3,4,9-tetrahydro-1H-carbazol-3-yl]pyrazolo[1,5-a][1,3,5]triazine-4,8-diamine (6.44 mg, 11.83 μmol, 6.37% yield, 98.81% purity, 3HCl, [α]^{27.9}_{D}=+6.304 (MeOH, c=0.021 g/100 mL) as a yellow solid. ¹H NMR (400 MHz, CD3OD) δ ppm 9.49 (br s, 1H), 8.76-8.69 (m, 2H), 8.31 (s, 1H), 7.35 (d, J=7.8 Hz, 1H), 7.26 (d, J=8.1 Hz, 1H), 7.02 (t, J=7.2 Hz, 1H), 6.96-6.90 (m, 1H), 4.95-4.92 (m, 1H), 3.55-3.34 (m, 1H), 3.26 (s, 3H), 3.12-3.01 (m, 1H), 3.01-2.89 (m, 2H), 2.43-2.33 (m, 1H), 2.32-2.20 (m, 1H); ES-LCMS m/z 429.2 [M+H]⁺.

### Example 55

### Synthesis of Compound I-52

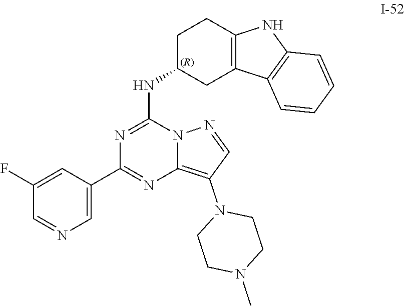

### Synthetic Scheme:

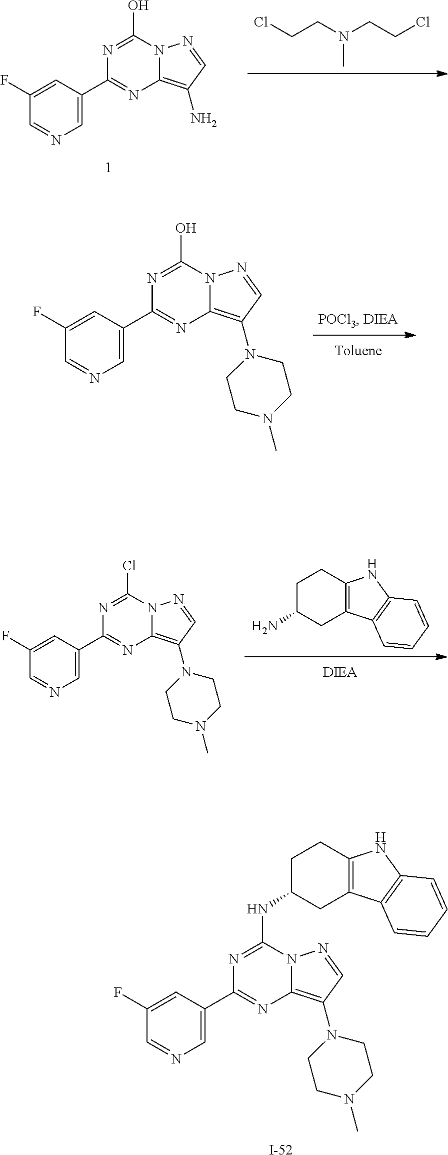

### Step 1: 2-(5-Fluoro-3-pyridyl)-8-(4-methylpiperazin-1-yl)pyrazolo[1,5-a][1,3,5]triazin-4-ol

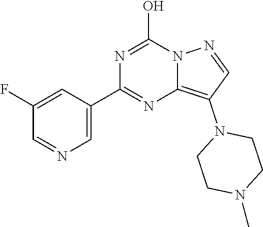 [SMILES: CN1CCN(c2cnn3c(O)nc(-c4cncc(F)c4)nc23)CC1]

To a stirred solution of 8-amino-2-(5-fluoro-3-pyridyl)pyrazolo[1,5-a][1,3,5]triazin-4-ol (800 mg, 2.80 mmol, 1 eq, HCl) in DMF (30 mL) was added Cs₂CO₃ (4.56 g, 14.01 mmol, 5 eq), NaI (1.26 g, 8.41 mmol, 3 eq) and 2-chloro-N-(2-chloroethyl)-N-methyl-ethanamine (1.08 g, 5.60 mmol, 2 eq, HCl). The reaction mixture was stirred at 90° C. for 12 h. The reaction mixture was concentrated to yield a residue under vacuum by oil pump. The residue was purified by preparative HPLC (basic condition; column: Xtimate C18 150*25 mm*5 um; mobile phase: [water (0.04% NH₃H₂O+10 mM NH₄HCO₃)−ACN]; B %: 0%-37%, 7.5 min) to yield 2-(5-fluoro-3-pyridyl)-8-(4-methylpiperazin-1-yl)pyrazolo[1,5-a][1,3,5]triazin-4-ol (75 mg, 182.19 μmol, 6.5% yield, 80.0% purity) as a yellow solid by lyophilization. ¹H NMR (400 MHz, CD3OD) δ ppm 9.25 (s, 1H), 8.52 (d, J=2.8 Hz, 1H), 8.41-8.38 (m, 1H), 7.74 (s, 1H), 4.82 (s, 1H), 3.56-3.45 (m, 4H), 3.13 (s, 4H), 2.70 (s, 3H).

### Step 2: 4-Chloro-2-(5-fluoro-3-pyridyl)-8-(4-methylpiperazin-1-yl)pyrazolo[1,5-a][1,3,5]triazine

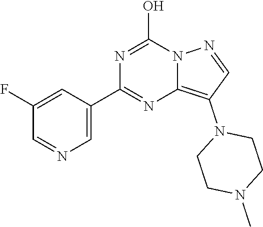 [SMILES: CN1CCN(c2cnn3c(O)nc(-c4cncc(F)c4)nc23)CC1]

A solution of 2-(5-fluoro-3-pyridyl)-8-(4-methylpiperazin-1-yl)pyrazolo[1,5-a][1,3,5]triazin-4-ol (75 mg, 182.19 μmol, 1 eq) in POCl₃ (8.27 g, 53.94 mmol, 5.01 mL, 296.04 eq) was stirred at 130° C. for 12 h. The reaction mixture was concentrated to yield a residue which was added to DCM (30 mL) and diluted with ice-water (50 mL). The mixture was extracted with DCM (30 mL×2) and washed with saturated KH₂PO₄ solution (20 mL×2). The combined organic layers were dried over Na₂SO₄, filtered and the filtrate was concentrated to yield 4-chloro-2-(5-fluoro-3-pyridyl)-8-(4-methylpiperazin-1-yl)pyrazolo[1,5-a][1,3,5]triazine (25 mg, 65.56 umol, 36.0% yield, 91.2% purity) as brown solid which was used in the next step without further purification. ES-LCMS m/z 348.1, 350.0 [M+H]⁺.

### Step 3: (3R)—N-[2-(5-Fluoro-3-pyridyl)-8-(4-methylpiperazin-1-yl)pyrazolo[1,5-a][1,3,5]triazin-4-yl]-2,3,4,9-tetrahydro-1H-carbazol-3-amine (I-52)

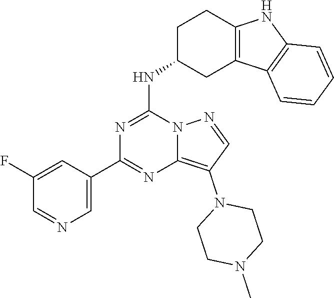 [SMILES: CN1CCN(c2cnn3c(N[C@@H]4CCc5[nH]c6ccccc6c5C4)nc(-c4cncc(F)c4)nc23)CC1]

To a solution of 4-chloro-2-(5-fluoro-3-pyridyl)-8-(4-methylpiperazin-1-yl)pyrazolo[1,5-a][1,3,5]triazine (25 mg, 65.56 μmol, 1 eq) in i-PrOH (3 mL) was added DIEA (42.36 mg, 327.80 μmol, 57.09 μL, 5 eq) and (3R)-2,3,4,9-tetrahydro-1H-carbazol-3-amine (12.82 mg, 68.84 μmol, 1.05 eq). The mixture was stirred at 60° C. for 1 h. The reaction mixture was concentrated to yield a residue which was purified by preparative HPLC (HCl condition; column: Agela ASB 150*25 mm*5 um; mobile phase: [water (0.05% HCl)−ACN]; B %: 30%-60%, 8 min). The desired fraction was lyophilized to yield (3R)—N-[2-(5-fluoro-3-pyridyl)-8-(4-methylpiperazin-1-yl)pyrazolo[1,5-a][1,3,5]triazin-4-yl]-2,3,4,9-tetrahydro-1H-carbazol-3-amine (12.22 mg, 18.99 μmol, 29.0% yield, 100.0% purity, 4HCl, [α]^{24.8}_{D}=+4.55 (MeOH, c=0.044 g/100 mL)) as a yellow solid. ¹H NMR (400 MHz, CD3OD) δ ppm 9.49 (s, 1H), 8.93 (d, J=9.3 Hz, 1H), 8.83 (s, 1H), 7.98 (s, 1H), 7.37 (d, J=7.6 Hz, 1H), 7.28 (d, J=8.1 Hz, 1H), 7.08-7.01 (m, 1H), 6.99-6.93 (m, 1H), 4.94 (s, 1H), 4.13 (d, J=13.0 Hz, 2H), 3.64 (d, J=11.5 Hz, 2H), 3.42-3.32 (m, 3H), 3.27 (s, 2H), 3.16-2.88 (m, 6H), 2.37 (d, J=3.2 Hz, 1H), 2.33-2.20 (m, 1H); ES-LCMS m/z 498.3 [M+H]⁺.

### Example 56

### Synthesis of Compound I-53

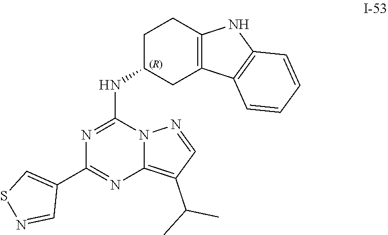

### Synthetic Scheme:

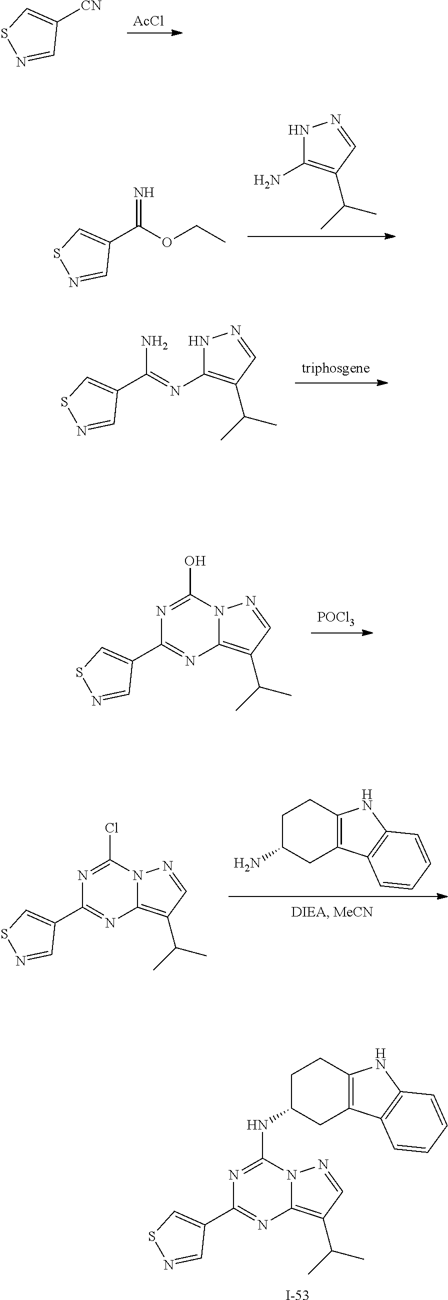

### Step 1: Ethyl isothiazole-4-carboximidate

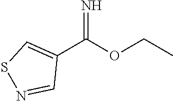

To DCM (4 mL) and EtOH (8 mL) was added acetyl chloride (4.56 g, 58.11 mmol, 4.15 mL, 8 eq) dropwise under N₂ atmosphere at 0° C. The mixture was stirred under N₂ atmosphere at 0° C. for 1 h. A solution of isothiazole-4-carbonitrile (800 mg, 7.26 mmol, 1 eq) in DCM (8 mL) was added dropwise under N₂ atmosphere at 0° C. The mixture was stirred under N₂ atmosphere at 25° C. for 12 h. TLC (PE/EtOAc=3/1, R_{f}=0.16) showed the starting material was consumed completely. The reaction mixture was concentrated under reduced pressure. The residue was diluted in EtOAc (50 mL) and poured into saturated aqueous NaHCO₃ (50 mL). The mixture was extracted with EtOAc (50 mL×3). The organic layer was dried over Na₂SO₄, filtered and concentrated under reduced pressure to yield ethyl isothiazole-4-carboximidate (973 mg, 5.62 mmol, 77.4% yield, 90.2% purity) as colorless oil, which was used in the next step without further purification. ¹H NMR (400 MHz, CDCl₃) δ ppm 8.98 (s, 1H), 8.78 (s, 1H), 4.30 (q, J=7.2 Hz, 2H), 1.42 (t, J=7.2 Hz, 3H); ES-LCMS m/z 157.2 [M+H]⁺.

### Step 2: N′-(4-Isopropyl-1H-pyrazol-5-yl)isothiazole-4-carboxamidine

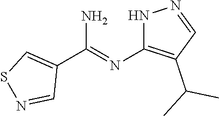 [SMILES: CC(C)c1cn[nH]c1/N=C(\N)c1cnsc1]

A mixture of ethyl isothiazole-4-carboximidate (973 mg, 5.62 mmol, 1 eq) and 4-isopropyl-1H-pyrazol-5-amine (703.28 mg, 5.62 mmol, 1 eq) in toluene (15 mL) was stirred at 115° C. for 12 h. TLC (PE/EtOAc=2/3, R_{f}=0.39) showed the starting material was consumed completely. The reaction mixture was concentrated under reduced pressure to yield a residue which was purified by flash silica gel chromatography (from PE/EtOAc=20/1 to 1/1, TLC: PE/EtOAc=2/3, R_{f}=0.39) to yield N-(4-isopropyl-1H-pyrazol-5-yl)isothiazole-4-carboxamidine (1.16 g, 4.76 mmol, 84.8% yield, 96.6% purity) as a colorless gum. ¹H NMR (400 MHz, CDCl₃) δ ppm 9.02 (s, 1H), 8.95 (s, 1H), 7.27 (s, 1H), 3.21-3.08 (m, 1H), 1.29 (d, J=6.8 Hz, 6H); ES-LCMS m/z 236.2 [M+H]⁺.

### Step 3: 8-Isopropyl-2-isothiazol-4-yl-pyrazolo[1,5-a][1,3,5]triazin-4-ol

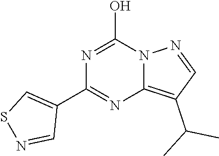 [SMILES: CC(C)c1cnn2c(O)nc(-c3cnsc3)nc12]

To a solution of N-(4-isopropyl-1H-pyrazol-5-yl)isothiazole-4-carboxamidine (1.16 g, 4.76 mmol, 1 eq) in 1,4-dioxane (10 mL) and THE (10 mL) was added diphosgene (984.00 mg, 4.97 mmol, 0.6 mL, 1.04 eq) dropwise at 25° C. The mixture was stirred at 25° C. for 15 minutes and then at 80° C. for 12 h. The reaction mixture was concentrated under reduced pressure. To the residue was added EtOAc (10 mL) and PE (50 mL). The mixture was stirred at 25° C. for 1 h and filtered. The solid was washed with PE/EtOAc (5/1, 10 mL) and dried under reduced pressure to yield 8-isopropyl-2-isothiazol-4-yl-pyrazolo[1,5-a][1,3,5]triazin-4-ol (1.15 g, 3.75 mmol, 78.6% yield, 97.1% purity, HCl) as a yellow solid. ¹H NMR (400 MHz, DMSO-d₆) δ ppm 12.72 (br s, 1H), 9.85 (s, 1H), 9.14 (s, 1H), 8.02 (s, 1H), 3.10 (m, 1H), 1.27 (d, J=6.8 Hz, 6H); ES-LCMS m z 262.1 [M+H]⁺.

### Step 4: 4-(4-Chloro-8-isopropyl-pyrazolo[1,5-a][1,3,5]triazin-2-yl)isothiazole

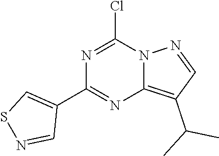 [SMILES: CC(C)c1cnn2c(Cl)nc(-c3cnsc3)nc12]

To a mixture of 8-isopropyl-2-isothiazol-4-yl-pyrazolo[1,5-a][1,3,5]triazin-4-ol (200 mg, 743.20 μmol, 1 eq) and DIEA (960.53 mg, 7.43 mmol, 1.29 mL, 10 eq) in toluene (10 mL) was added POCl₃ (1.14 g, 7.43 mmol, 690.64 μL, 10 eq) at 25° C. The mixture was stirred at 130° C. for 2 h. The residue was dissolved in EtOAc (30 mL), poured into saturated aqueous KH₂PO₃ (30 mL) and extracted with EtOAc (30 mL×3). The organic layer was dried over Na₂SO₄, filtered and concentrated under reduced pressure to yield 4-(4-chloro-8-isopropyl-pyrazolo[1,5-a][1,3,5]triazin-2-yl)isothiazole (140 mg, 472.42 μmol, 63.6% yield, 94.4% purity) as a brown solid, which was used in the next step without further purification. ¹H NMR (400 MHz, CDCl₃) δ ppm 9.50 (s, 1H), 9.28 (s, 1H), 8.16 (s, 1H), 3.40-3.28 (m, 1H), 1.43 (d, J=7.2 Hz, 6H); ES-LCMS m/z 280.1, 282.1 [M+H]⁺.

### Step 5: (3R)—N-(8-Isopropyl-2-isothiazol-4-yl-pyrazolo[1,5-a][1,3,5]triazin-4-yl)-2,3,4,9-tetrahydro-1H-carbazol-3-amine (I-53)

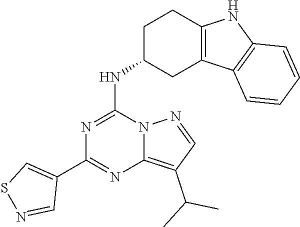 [SMILES: CC(C)c1cnn2c(N[C@@H]3CCc4[nH]c5ccccc5c4C3)nc(-c3cnsc3)nc12]

A mixture of 4-(4-chloro-8-isopropyl-pyrazolo[1,5-a][1,3,5]triazin-2-yl)isothiazole (80 mg, 269.96 μmol, 1 eq), (3R)-2,3,4,9-tetrahydro-1H-carbazol-3-amine (56 mg, 300.67 μmol, 1.11 eq) and DIEA (174.45 mg, 1.35 mmol, 235.11 μL, 5 eq) in MeCN (10 mL) was stirred at 60° C. for 12 h. The reaction mixture was concentrated under reduced pressure to yield a residue which was purified by preparative HPLC (column: Agela ASB 150*25 mm*5 um; mobile phase: [water (0.05% HCl)−ACN]; B %: 68%-98%, 8 min) to yield (3R)—N-(8-isopropyl-2-isothiazol-4-yl-pyrazolo[1,5-a][1,3,5]triazin-4-yl)-2,3,4,9-tetrahydro-1H-carbazol-3-amine (42.45 mg, 91.09 μmol, 33.7% yield, 100.0% purity, HCl, [α]^{24.6}_{D}=+33.96 (DMSO, c=0.106 g/100 mL)) as a yellow solid. ¹H NMR (400 MHz, CD3OD) δ ppm 9.60 (s, 1H), 9.18 (s, 1H), 7.98 (s, 1H), 7.36 (d, J=8.0 Hz, 1H), 7.25 (d, J=8.0 Hz, 1H), 7.05-6.99 (m, 1H), 6.96-6.91 (m, 1H), 4.85-4.82 (m, 1H), 3.28-3.22 (m, 2H), 3.07-2.88 (m, 3H), 2.42-2.31 (m, 1H), 2.29-2.18 (m, 1H), 1.37 (d, J=6.8 Hz, 6H); ES-LCMS m/z 430.2 [M+H]⁺.

### Example 57

### Synthesis of Compound I-54

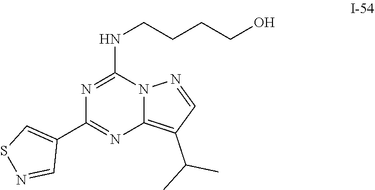 [SMILES: CC(C)c1cnn2c(NCCCCO)nc(-c3cnsc3)nc12]

### Synthetic Scheme:

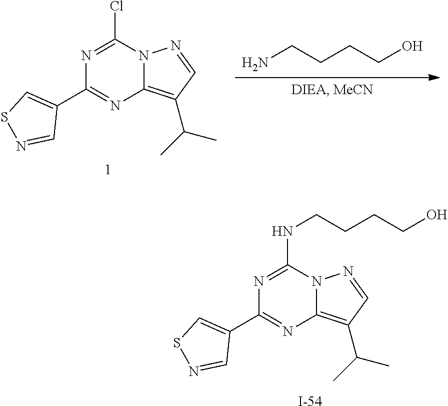

### Step 1: 4-[(8-Isopropyl-2-isothiazol-4-yl-pyrazolo[1,5-a][1,3,5]triazin-4-yl)amino]butan-1-ol (I-54)

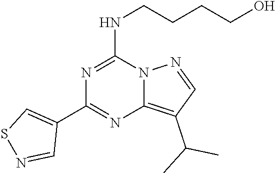 [SMILES: CC(C)c1cnn2c(NCCCCO)nc(-c3cnsc3)nc12]

A mixture of 4-(4-chloro-8-isopropyl-pyrazolo[1,5-a][1,3,5]triazin-2-yl)isothiazole (60 mg, 202.47 μmol, 1 eq), 4-aminobutan-1-ol (20 mg, 224.38 μmol, 20.83 μL, 1.11 eq) and DIEA (130.84 mg, 1.01 mmol, 176.33 μL, 5 eq) in MeCN (10 mL) was stirred at 60° C. for 12 h. The reaction mixture was concentrated under reduced pressure to yield a residue which was purified by preparative HPLC (column: Agela ASB 150*25 mm*5 um; mobile phase: [water (0.05% HCl)−ACN]; B %: 45%-75%, 8 min) to yield 4-[(8-isopropyl-2-isothiazol-4-yl-pyrazolo[1,5-a][1,3,5]triazin-4-yl)amino]butan-1-ol (48.10 mg, 130.39 μmol, 64.4% yield, 100.0% purity, HCl) as a white solid. ¹H NMR (400 MHz, CD3OD) δ ppm 9.82 (s, 1H), 9.24 (s, 1H), 8.08 (s, 1H), 3.83 (t, J=7.2 Hz, 2H), 3.62 (t, J=6.4 Hz, 2H), 3.34-3.30 (m, 1H), 1.90-1.83 (m, 2H), 1.71-1.63 (m, 2H), 1.36 (d, J=7.2 Hz, 6H); ES-LCMS m/z 333.2 [M+H]⁺.

### Example 58

### Synthesis of Compound I-55a, I-55b and I-55c

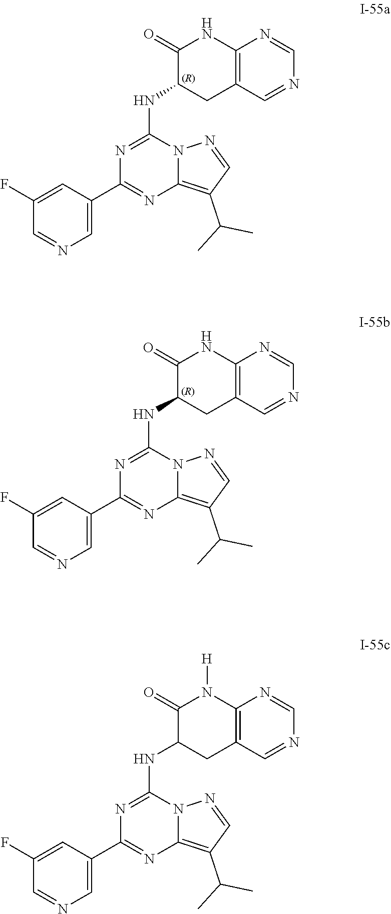

### Synthetic Scheme:

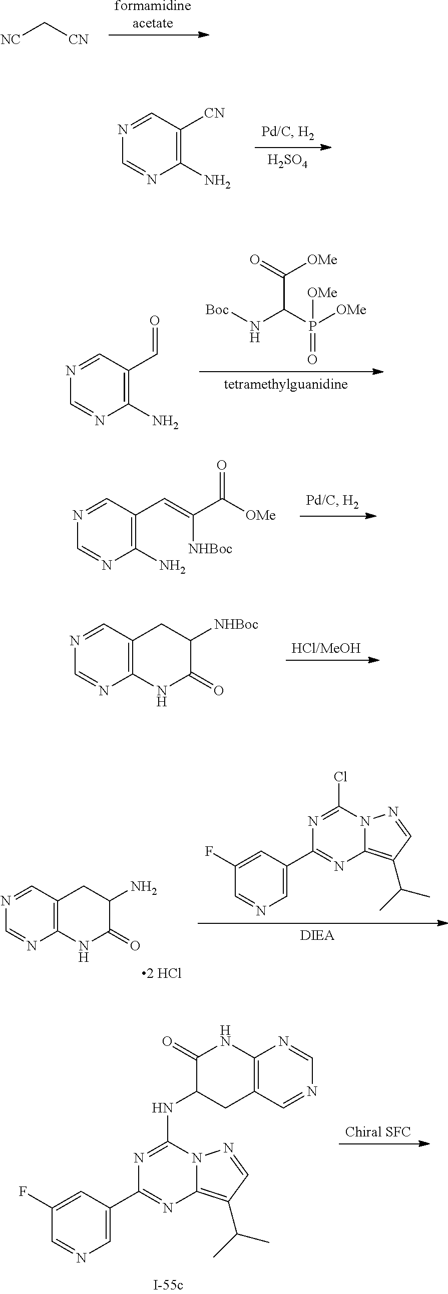

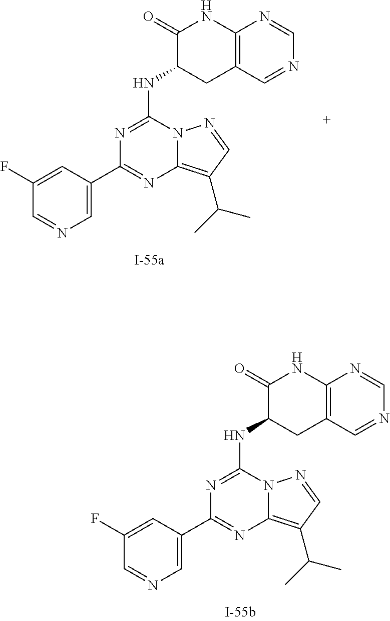 [SMILES: CC(C)c1cnn2c(N[C@H]3Cc4cncnc4NC3=O)nc(-c3cncc(F)c3)nc12 ; CC(C)c1cnn2c(N[C@@H]3Cc4cncnc4NC3=O)nc(-c3cncc(F)c3)nc12]

### Step 1: 4-Aminopyrimidine-5-carbonitrile

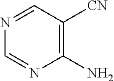

To MeOH (900 mL) was added Na (17.40 g, 756.87 mmol, 17.94 mL, 2 eq) partwise at 0° C. The mixture was stirred at 0° C. until Na disappeared to yield NaOMe solution. To a mixture of propanedinitrile (25 g, 378.44 mmol, 23.81 mL, 1 eq) and formamidine acetate (78.80 g, 756.87 mmol, 2 eq) in MeOH (100 mL) was added above NaOMe solution dropwise at 25° C. The mixture was stirred at 25° C. for 15 h. The mixture was concentrated under reduced pressure to about half of the original and then stirred at 25° C. for 15 h. The reaction mixture was filtered. The solid was washed with MeOH (150 mL) and then dried under reduced pressure to yield 4-aminopyrimidine-5-carbonitrile (23.4 g, 194.82 mmol, 51.5% yield) as a yellow solid, which was used in the next step without further purification. ¹H NMR (400 MHz, DMSO-d₆) δ ppm 8.60 (s, 1H), 8.54 (s, 1H), 7.95 (br s, 2H).

### Step 2: 4-Aminopyrimidine-5-carbaldehyde

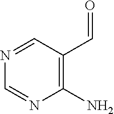

To 4-aminopyrimidine-5-carbonitrile (1 g, 8.33 mmol, 1 eq) was added H₂O (10 mL) and H₂SO₄ (2 mL) at 25° C. Then to the solution was added Pd/C (200 mg, 10% purity) at 25° C. under N₂ atmosphere. The mixture was stirred under H₂ (15 Psi) at 25° C. for 12 h. TLC (PE/EtOAc=1/1, R_{f}=0.52) showed the starting material was consumed completely. The mixture was filtered through a bed of Celite to remove the catalyst. The clear, pale yellow filtrate was treated with concentrated ammonium hydroxide to neutralize the acid. After chilling, the solid was collected, washed with cold water (15 mL) and dried under reduced pressure to yield 4-aminopyrimidine-5-carbaldehyde (0.16 g, 1.30 mmol, 15.6% yield) as a yellow solid, which was used in the next step without further purification. ¹H NMR (400 MHz, DMSO-d₆) δ ppm 9.87 (s, 1H), 8.74 (s, 1H), 8.56 (s, 1H), 8.28 (br s, 1H), 7.98 (br s, 1H).

### Step 3: Methyl (Z)-3-(4-aminopyrimidin-5-yl)-2-(tert-butoxycarbonylamino)prop-2-enoate

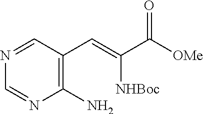 [SMILES: COC(=O)/C(=C/c1cncnc1N)NC(=O)OC(C)(C)C]

To a solution of methyl 2-(tert-butoxycarbonylamino)-2-dimethoxyphosphoryl-acetate (2.41 g, 8.12 mmol, 1 eq) in THE (80 mL) was added tetramethylguanidine (935.54 mg, 8.12 mmol, 1 eq) slowly at −70° C. under N₂. After being 15 min, a solution of 4-aminopyrimidine-5-carbaldehyde (1 g, 8.12 mmol, 1 eq) in THE (60 mL) was added drop-wise at −70° C. The resulting mixture was stirred for 12 h at 25° C. The reaction mixture was quenched by addition of water (50 mL), extracted with EtOAc (40 mL×3). The combined organic layers were washed with brine (40 mL), dried over Na₂SO₄, filtered and concentrated under reduced pressure to give a residue which was purified by flash silica gel chromatography (from PE/EtOAc=1/0 to 0/1, TLC: PE/EtOAc=1/1, R_{f}=0.15) to yield methyl (Z)-3-(4-aminopyrimidin-5-yl)-2-(tert-butoxycarbonylamino)prop-2-enoate (1.14 g, 3.33 mmol, 41.1% yield, 86.0% purity) as a yellow solid. ¹H NMR (400 MHz, DMSO-d₆) δ ppm 8.73 (d, J=12.5 Hz, 1H), 8.31 (s, 1H), 8.28 (s, 1H), 7.07-6.91 (m, 3H), 3.74 (s, 3H), 1.37 (s, 9H); ES-LCMS m/z 295.2 [M+H]⁺.

### Step 4: tert-Butyl N-(7-oxo-6,8-dihydro-5H-pyrido[2,3-d]pyrimidin-6-yl)carbamate

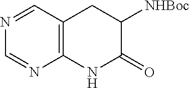 [SMILES: CC(C)(C)OC(=O)NC1Cc2cncnc2NC1=O]

To a solution of methyl (Z)-3-(4-aminopyrimidin-5-yl)-2-(tert-butoxycarbonylamino)prop-2-enoate (240 mg, 815.48 μmol, 1 eq) in EtOH (15 mL) was added Pd/C (50 mg, 2.38 mmol, 10% purity) under N₂ atmosphere. The mixture was stirred for 16 h at 25° C. under H₂ (30 psi) atmosphere. The mixture was filtered and the filtrate was concentrated to yield tert-butyl N-(7-oxo-6,8-dihydro-5H-pyrido[2,3-d]pyrimidin-6-yl)carbamate (150 mg, 539.20 μmol, 66.1% yield, 95.0% purity) as an off-white solid, which was used in the next step without purification. ¹H NMR (400 MHz, CDCl₃) δ ppm 8.76 (s, 1H), 8.41 (s, 1H), 5.58-5.48 (m, 1H), 4.43-4.29 (m, 1H), 3.54 (d, J=9.0 Hz, 1H), 2.76 (t, J=14.7 Hz, 1H), 1.42 (s, 9H); ES-LCMS m/z 265.1 [M+H]⁺.

### Step 5: 6-Amino-6,8-dihydro-5H-pyrido[2,3-d]pyrimidin-7-one

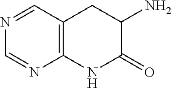 [SMILES: NC1Cc2cncnc2NC1=O]

To a solution of tert-butyl N-(7-oxo-6,8-dihydro-5H-pyrido[2,3-d]pyrimidin-6-yl)carbamate (150 mg, 539.20 μmol, 1 eq) in CH₂Cl₂ (6 mL) was added HCl/MeOH (4 M, 3 mL) and the mixture was stirred at 25° C. for 1 h. TLC (PE/EtOAc=1/1, R_{f}=0.1) showed the starting material was consumed completely. The reaction mixture was concentrated under reduced pressure to yield 6-amino-6,8-dihydro-5H-pyrido[2,3-d]pyrimidin-7-one (83.7 mg, 353.04 μmol, 65.5% yield, N/A purity, 2HCl) as a white solid, which was used in the next step without further purification. ¹H NMR (400 MHz, DMSO-d₆) δ ppm 11.69 (s, 1H), 8.80 (s, 1H), 8.77 (br s, 2H), 8.63 (s, 1H), 4.49-4.36 (m, 1H), 3.33 (dd, J=7.0, 15.6 Hz, 1H), 3.18-3.06 (m, 1H).

### Step 6: 6S)-6-[[2-(5-Fluoro-3-pyridyl)-8-isopropyl-pyrazolo[1,5-a][1,3,5]triazin-4-yl]amino]-6,8-dihydro-5H-pyrido[2,3-d]pyrimidin-7-one (I-55a) and (6R)-6-[[2-(5-fluoro-3-pyridyl)-8-isopropyl-pyrazolo[1,5-a][1,3,5]triazin-4-yl]amino]-6,8-dihydro-5H-pyrido[2,3-d]pyrimidin-7-one (I-55b)

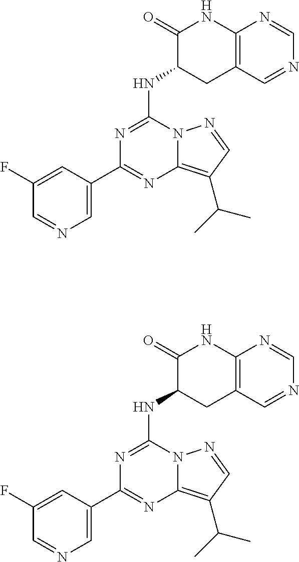 [SMILES: CC(C)c1cnn2c(N[C@H]3Cc4cncnc4NC3=O)nc(-c3cncc(F)c3)nc12 ; CC(C)c1cnn2c(N[C@@H]3Cc4cncnc4NC3=O)nc(-c3cncc(F)c3)nc12]

A mixture of 6-amino-6,8-dihydro-5H-pyrido[2,3-d]pyrimidin-7-one (80 mg, 337.43 μmol, 1 eq, 2HCl), 4-chloro-2-(5-fluoro-3-pyridyl)-8-isopropyl-pyrazolo[1,5-a][1,3,5]triazine (103.39 mg, 337.43 μmol, 1 eq), DIEA (218.05 mg, 1.69 mmol, 293.86 μL, 5 eq) in i-PrOH (10 mL) was stirred at 60° C. for 2 h under N₂ atmosphere. The reaction mixture was concentrated under reduced pressure to yield a residue which was purified by flash silica gel chromatography (from PE/EtOAc=1/0 to 1/1, TLC: PE/EtOAc=1/1, R_{f}=0.12). The product was separated by chiral SFC (column: DAICEL CHIRALPAK AD (250 mm*30 mm, 10 um); mobile phase: [0.1% NH₃.H₂O/EtOH]; B %: 50%-50%) to yield Peak 1 and Peak 2. One of these peaks was concentrated under reduced pressure at 25° C. to yield an enantiomer (34 mg, 81.07 μmol, 24.1% yield, 100.0% purity, SFC: Rₜ=4.176, ee=75.726%, [α]^{25.1}_{D}=6.67 (DMSO, c=0.030 g/100 mL)) as a white solid. ¹H NMR (400 MHz, DMSO-d₆) δ ppm 11.23 (br s, 1H), 9.39 (s, 1H), 9.14 (br s, 1H), 8.71 (s, 1H), 8.67 (d, J=2.9 Hz, 1H), 8.54-8.47 (m, 2H), 8.18 (s, 1H), 5.76-5.66 (m, 1H), 3.43 (t, J=14.1 Hz, 1H), 3.24-3.18 (m, 2H), 1.35 (d, J=6.8 Hz, 6H); ES-LCMS m/z 420.1 [M+H]⁺. The other of these peaks was concentrated under reduced pressure at 25° C. to yield the other enantiomer (26 mg, 61.99 μmol, 18.4% yield, 100.0% purity, SFC: Rₜ=5.927, ee=48.742%, [α]25.2_{D}=−13.33 (DMSO, c=0.030 g/100 mL)) as a white solid. ¹H NMR (400 MHz, DMSO-d₆) δ ppm 11.22 (br s, 1H), 9.39 (s, 1H), 9.14 (br s, 1H), 8.71 (s, 1H), 8.66 (d, J=2.7 Hz, 1H), 8.54-8.46 (m, 2H), 8.18 (s, 1H), 5.76-5.66 (m, 1H), 3.48-3.35 (m, 1H), 3.24-3.16 (m, 2H), 1.35 (d, J=6.8 Hz, 6H); ES-LCMS m/z 420.2 [M+H]⁺.

### Example 59

### Synthesis of Compound I-56a, I-56b and I-56c

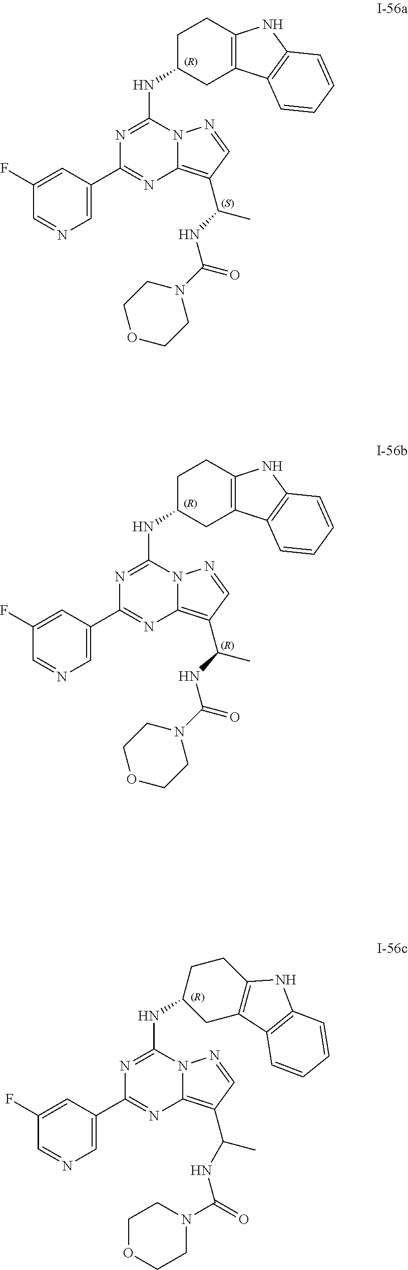

### Synthetic Scheme:

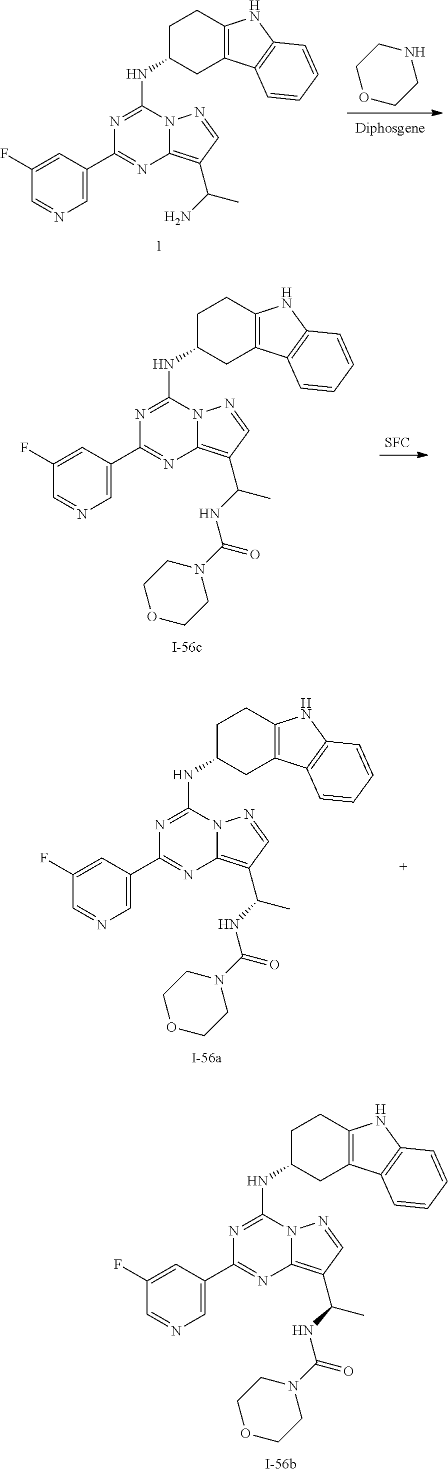

### Step 1: N-[(1S)-1-[2-(5-Fluoro-3-pyridyl)-4-[[(3R)-2,3,4,9-tetrahydro-1H-carbazol-3-yl]amino]pyrazolo[1,5-a][1,3,5]triazin-8-yl]ethyl]morpholine-4-carboxamide (I-56a) and N-[(1R)-1-[2-(5-Fluoro-3-pyridyl)-4-[[(3R)-2,3,4,9-tetrahydro-1H-carbazol-3-yl]amino]pyrazolo[1,5-a][1,3,5]triazin-8-yl]ethyl]morpholine-4-carboxamide (I-56b)

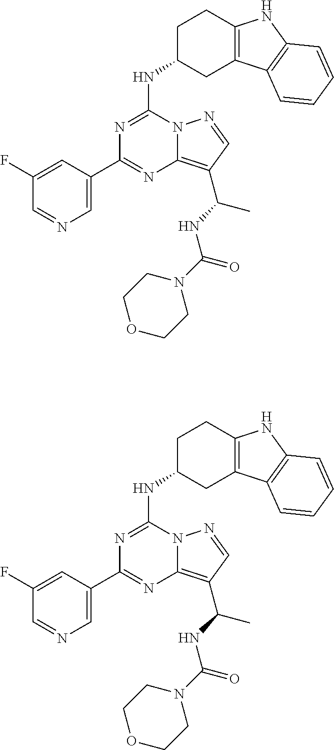 [SMILES: C[C@H](NC(=O)N1CCOCC1)c1cnn2c(N[C@@H]3CCc4[nH]c5ccccc5c4C3)nc(-c3cncc(F)c3)nc12 ; C[C@@H](NC(=O)N1CCOCC1)c1cnn2c(N[C@@H]3CCc4[nH]c5ccccc5c4C3)nc(-c3cncc(F)c3)nc12]

To a stirred solution of (3R)—N-[8-(1-aminoethyl)-2-(5-fluoro-3-pyridyl)pyrazolo[1,5-a][1,3,5]triazin-4-yl]-2,3,4,9-tetrahydro-1H-carbazol-3-amine (210 mg, 407.20 μmol, 1 eq) in THE (10 mL) was added trichloromethyl carbonochloridate (402.78 mg, 2.04 mmol, 245.60 μL, 5 eq). The reaction mixture was stirred at 55° C. for 3 h. Morpholine (2.72 g, 31.26 mmol, 2.75 mL, 76.77 eq) was added to the above reaction mixture and stirred at 55° C. for 12 h. The reaction mixture was concentrated to give a residue which was purified by preparative HPLC (basic condition; column: Boston Green ODS 150*30 5 u; mobile phase: [water (0.04% NH₃H₂O+10 mM NH₄HCO₃)−ACN]; B %: 45%-75%, 8 min) to yield the product which was separated by SFC (column: DAICEL CHIRALCEL OJ (250 mm*30 mm, 10 um); mobile phase: [0.1% NH₃H₂O ETOH]; B %: 40%-40%, min) to yield an enantiomer (15.15 mg, 26.72 μmol, 6.6% yield, 98.0% purity, SFC: Rₜ=2.759 min, ee=98.0%, [α]^{22.7}_{D}=+2.0, MeOH, c=0.100 g/100 mL) by lyophilization as a white solid; ¹H NMR (400 MHz, CD3OD) δ ppm 9.42 (s, 1H), 8.60-8.51 (m, 2H), 8.04 (s, 1H), 7.38 (d, J=7.6 Hz, 1H), 7.27 (d, J=8.1 Hz, 1H), 7.06-7.01 (m, 1H), 6.98-6.93 (m, 1H), 5.30 (q, J=7.0 Hz, 1H), 4.92 (s, 1H), 3.66-3.62 (m, 4H), 3.41 (q, J=4.6 Hz, 4H), 3.28 (s, 1H), 3.15-2.89 (m, 3H), 2.37 (s, 1H), 2.32-2.21 (m, 1H), 1.66 (d, J=7.1 Hz, 3H); ES-LCMS m/z 556.3 [M+H]⁺ and the other enantiomer (42.97 mg, 75.87 μmol, 18.6% yield, 98.1% purity, SFC: Rₜ=3.583 min, ee=100.0%, [α]^{22.7}_{D}=+16.5, MeOH, c=0.097 g/100 mL) by lyophilization as a white solid; ¹H NMR (400 MHz, CD3OD) δ ppm 9.42 (s, 1H), 8.60-8.50 (m, 2H), 8.04 (s, 1H), 7.38 (d, J=7.8 Hz, 1H), 7.27 (d, J=8.1 Hz, 1H), 7.07-7.00 (m, 1H), 6.99-6.92 (m, 1H), 5.30 (q, J=6.8 Hz, 1H), 4.96-4.89 (m, 1H), 3.66-3.61 (m, 4H), 3.45-3.37 (m, 4H), 3.28 (d, J=5.1 Hz, 1H), 3.10-2.89 (m, 3H), 2.38 (d, J=6.4 Hz, 1H), 2.32-2.20 (m, 1H), 1.66 (d, J=7.1 Hz, 3H); ES-LCMS m/z 556.3 [M+H]⁺.

### Example 60

### Synthesis of Compound I-58

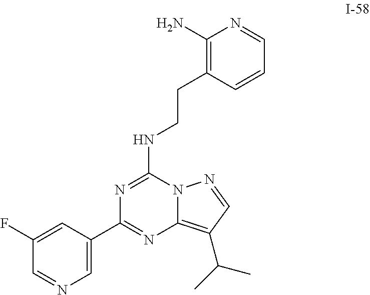 [SMILES: CC(C)c1cnn2c(NCCc3cccnc3N)nc(-c3cncc(F)c3)nc12]

### Synthetic Scheme:

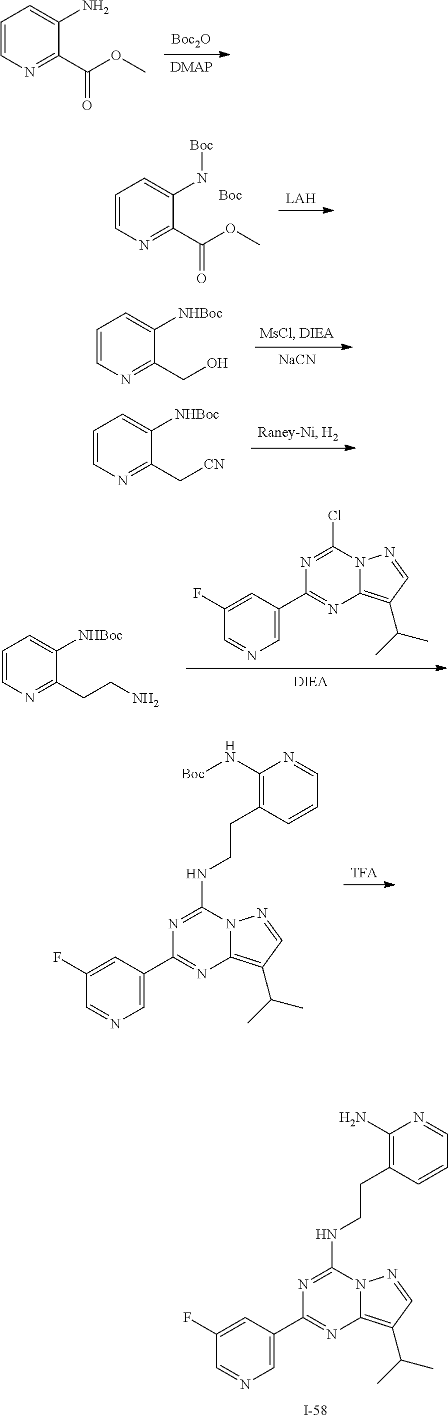

### Step 1: Methyl 3-[bis(tert-butoxycarbonyl)amino]pyridine-2-carboxylate

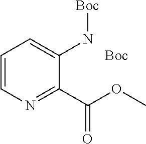 [SMILES: COC(=O)c1ncccc1NC(=O)OC(C)(C)C ; "Boc"]

To a solution of methyl 3-aminopyridine-2-carboxylate (5 g, 32.86 mmol, 1 eq) in DCM (100 mL) were added Boc₂O (21.52 g, 98.59 mmol, 22.65 mL, 3 eq) and DMAP (200.74 mg, 1.64 mmol, 0.05 eq). The mixture was stirred at 25° C. for 36 h. The reaction mixture was concentrated under reduced pressure. The residue was diluted with water (50 mL) and extracted with EtOAc (50 mL×2). The combined organic layers were washed with brine (30 mL), dried over Na₂SO₄, filtered and concentrated under reduced pressure to give a residue which was purified by flash silica gel chromatography (from PE/EtOAc=1/0 to 5/1) to yield methyl 3-[bis(tert-butoxycarbonyl)amino]pyridine-2-carboxylate (7.4 g, 18.90 mmol, 57.5% yield, 90.0% purity) as a white solid. ¹H NMR (400 MHz, CD3OD) δ ppm 8.63 (dd, J=4.8, 2.0 Hz, 1H), 8.45 (dd, J=7.8, 1.9 Hz, 1H), 7.56 (dd, J=7.8, 5.0 Hz, 1H), 3.90 (s, 3H), 1.32 (s, 18H).

### Step 2: tert-Butyl (2-(hydroxymethyl)pyridin-3-yl)carbamate

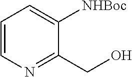 [SMILES: CC(C)(C)OC(=O)Nc1cccnc1CO]

To a solution of methyl 3-[bis(tert-butoxycarbonyl)amino]pyridine-2-carboxylate (5.3 g, 13.54 mmol, 1 eq) in THE (100 mL) was added LiAlH₄ (770.56 mg, 20.30 mmol, 1.5 eq). The mixture was stirred at 0° C. for 2 h. The reaction mixture was quenched by addition water (30 mL) at 0° C., and then diluted with water (50 mL) and extracted with EtOAc (50 mL×3). The combined organic layers were washed with brine (30 mL), dried over Na₂SO₄, filtered and concentrated under reduced pressure to yield tert-butyl [2-(hydroxymethyl)-3-pyridyl]carbamate (3.1 g, 12.44 mmol, 91.9% yield, 90.0% purity) as a white solid. ¹H NMR (400 MHz, CD3OD) δ ppm 8.23 (dd, J=5.0, 1.6 Hz, 1H), 7.87-7.82 (m, 1H), 7.20 (dd, J=7.6, 5.0 Hz, 1H), 4.59 (s, 2H), 1.50 (s, 9H).

### Step 3: tert-Butyl (2-(cyanomethyl)pyridin-3-yl)carbamate

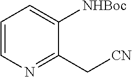 [SMILES: CC(C)(C)OC(=O)Nc1cccnc1CC#N]

To a solution of tert-butyl N-[2-(hydroxymethyl)-3-pyridyl]carbamate (1.9 g, 7.63 mmol, 1 eq) in DCM (20 mL) were added DIEA (2.3 g, 17.80 mmol, 3.10 mL, 2.33 eq) and MsCl (1.72 g, 15.02 mmol, 1.16 mL, 1.97 eq). The mixture was stirred at 0° C. for 10 min, then the mixture was concentrated under reduced pressure. The residue was dissolved in DMF (20 mL) and NaCN (2.60 g, 53.05 mmol, 6.96 eq) was added. The mixture was stirred at 25° C. for 12 h. The reaction mixture was basified pH˜8 with sat. NaHCO₃ (˜50 mL), diluted with water (100 mL) and extracted with EtOAc (50 mL×2). The combined organic layers were washed with brine (30 mL), dried over Na₂SO₄, filtered and concentrated under reduced pressure to give a residue which was purified by flash silica gel chromatography (from PE/EtOAc=1/0 to 5/1) to yield compound tert-butyl N-[2-(cyanomethyl)-3-pyridyl]carbamate (600 mg, 2.31 mmol, 30.4% yield, 90.0% purity) as a white solid. ¹H NMR (400 MHz, CD3OD) δ ppm 8.36-8.30 (m, 1H), 7.90 (d, J=7.7 Hz, 1H), 7.30 (dd, J=7.7, 4.8 Hz, 1H), 3.93 (s, 2H), 1.51 (s, 9H).

### Step 4: tert-Butyl N-[2-(2-aminoethyl)-3-pyridyl]carbamate

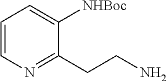 [SMILES: CC(C)(C)OC(=O)Nc1cccnc1CCN]

To a solution of tert-butyl N-[2-(cyanomethyl)-3-pyridyl]carbamate (600 mg, 2.31 mmol, 1 eq) in MeOH (15 mL) was added raney nickel (198.32 mg, 2.31 mmol, 1 eq) under Ar. The suspension was degassed under vacuum and purged with H₂ several times. The mixture was stirred under H₂ (15 psi) at 25° C. for 12 h. The reaction mixture was filtered through a ped of celite. The filtrate was concentrated under reduced pressure to yield a residue which was purified by preparative TLC (DCM/MeOH=10/1, R_{f}=0.10) to yield compound tert-butyl N-[2-(2-aminoethyl)-3-pyridyl]carbamate (75 mg, 284.45 μmol, 12.3% yield, 90.0% purity) as a yellow solid. ¹H NMR (400 MHz, CD3OD) δ ppm 8.25 (d, J=5.1 Hz, 1H), 7.76-7.68 (m, 1H), 7.23 (dd, J=7.5, 4.8 Hz, 1H), 2.91-2.98 (m, 2H), 2.86-2.77 (m, 2H), 1.50 (s, 9H); ES-LCMS m/z 238.3 [M+H]⁺.

### Step 5: tert-Butyl N-[3-[2-[[2-(5-fluoro-3-pyridyl)-8-isopropyl-pyrazolo[1,5-a][1,3,5]triazin-4-yl]amino]ethyl]-2-pyridyl]carbamate

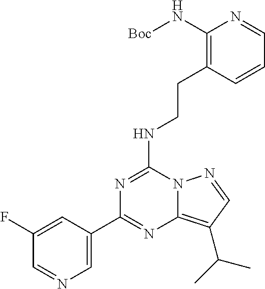 [SMILES: CC(C)c1cnn2c(NCCc3cccnc3NC(=O)OC(C)(C)C)nc(-c3cncc(F)c3)nc12]

To a solution of 4-chloro-2-(5-fluoro-3-pyridyl)-8-isopropyl-pyrazolo[1,5-a][1,3,5]triazine (130 μmg, 445.65 μmol, 1.57 eq), tert-butyl N-[2-(2-aminoethyl)-3-pyridyl]carbamate (75 mg, 284.45 μmol, 1 eq) in i-PrOH (6 mL) was added DIEA (148.40 mg, 1.15 mmol, 200 μL, 4.04 eq). The mixture was stirred at 60° C. for 1 h. The reaction mixture was concentrated under reduced pressure to yield a residue which was purified by preparative TLC (PE/EtOAc=1/1) to yield tert-butyl N-[3-[2-[[2-(5-fluoro-3-pyridyl)-8-isopropyl-pyrazolo[1,5-a][1,3,5]triazin-4-yl]amino]ethyl]-2-pyridyl]carbamate (75 mg, 152.27 μmol) as a yellow solid. ES-LCMS m/z 493.3 [M+H]⁺.

### Step 6: N-[2-(2-Amino-3-pyridyl)ethyl]-2-(5-fluoro-3-pyridyl)-8-isopropyl-pyrazolo[1,5-a][1,3,5]triazin-4-amine (I-58)

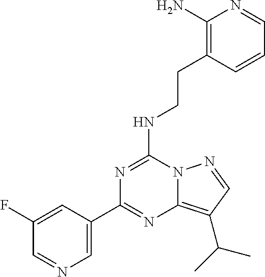 [SMILES: CC(C)c1cnn2c(NCCc3cccnc3N)nc(-c3cncc(F)c3)nc12]

To a solution of tert-butyl N-[3-[2-[[2-(5-fluoro-3-pyridyl)-8-isopropyl-pyrazolo[1,5-a][1,3,5]triazin-4-yl]amino]ethyl]-2-pyridyl]carbamate (75 mg, 152.27 μmol, 1 eq) in DCM (6 mL) was added TFA (3.85 g, 33.77 mmol, 2.5 mL, 221.75 eq). The mixture was stirred at 25° C. for 1 h. The reaction mixture was concentrated under reduced pressure to give a residue which was purified by preparative HPLC (column: Xtimate C18 150*25 mm*5 um; mobile phase: [water (0.05% HCl)−ACN]; B %: 28%-58%, 8 min) followed by freeze drying to yield N-[2-(2-amino-3-pyridyl)ethyl]-2-(5-fluoro-3-pyridyl)-8-isopropyl-pyrazolo[1,5-a][1,3,5]triazin-4-amine (15.66 mg, 31.21 μmol, 20.5% yield, 100.0% purity, 3 HCl) as a yellow solid. ¹H NMR (400 MHz, CD3OD) δ ppm 9.45 (s, 1H), 9.01-8.88 (m, 2H), 8.00 (s, 1H), 7.78 (d, J=7.3 Hz, 1H), 7.63 (d, J=6.4 Hz, 1H), 6.72 (t, J=6.8 Hz, 1H), 4.08 (t, J=6.4 Hz, 2H), 3.19-3.27 (m, 1H), 3.10 (t, J=6.4 Hz, 2H), 1.38 (d, J=7.1 Hz, 6H); ES-LCMS m/z 393.2 [M+H]⁺.

### Example 61

### Synthesis of Compound I-59

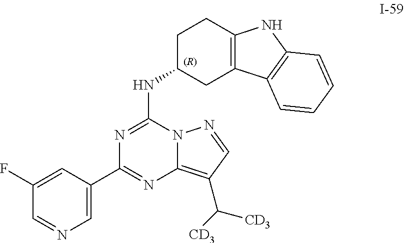

### Synthetic Scheme:

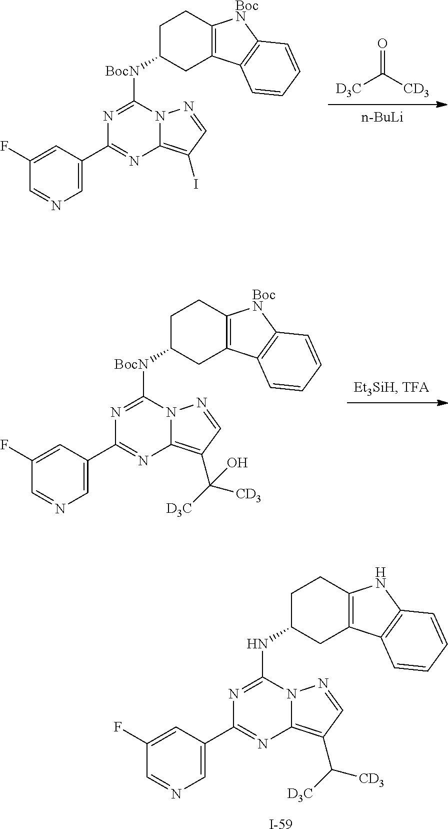

### Step 1: tert-Butyl (3R)-3-[tert-butoxycarbonyl-[2-(5-fluoro-3-pyridyl)-8-[2,2,2-trideuterio-1-hydroxy-1-(trideuteriomethyl)ethyl]pyrazolo[1,5-a][1,3,5]triazin-4-yl]amino]-1,2,3,4-tetrahydrocarbazole-9-carboxylate

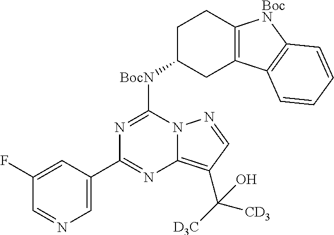 [SMILES: [2H]C([2H])([2H])C(O)(c1cnn2c(N(C(=O)OC(C)(C)C)[C@@H]3CCc4c(c5ccccc5n4C(=O)OC(C)(C)C)C3)nc(-c3cncc(F)c3)nc12)C([2H])([2H])[2H]]

To a solution of tert-butyl (3R)-3-[tert-butoxycarbonyl-[2-(5-fluoro-3-pyridyl)-8-iodo-pyrazolo[1,5-a][1,3,5]triazin-4-yl]amino]-1,2,3,4-tetrahydrocarbazole-9-carboxylate (500.00 mg, 689.13 μmol, 1 eq) in THE (20 mL) was added n-BuLi (2.5 M, 0.4 mL, 1.45 eq) dropwise under N₂ atmosphere at −78° C. The mixture was stirred under N₂ atmosphere at −78° C. for 0.5 h. 1,1,1,3,3,3-hexadeuteriopropan-2-one (130 mg, 2.03 mmol, 149.08 μL, 2.94 eq) was added dropwise under N₂ atmosphere at −78° C. The mixture was stirred under N₂ atmosphere at −78° C. for 1 h. TLC (PE/EtOAc=3/1, R_{f}=0.40) showed the starting material was consumed completely. The reaction mixture was quenched with water (30 mL) and extracted with EtOAc (30 mL×3). The organic layer was dried over Na₂SO₄, filtered and concentrated under reduced pressure to yield a residue which was purified by flash silica gel chromatography (from PE/EtOAc=20/1 to 4/1, TLC: PE/EtOAc=3/1, R_{f}=0.40) to yield tert-butyl (3R)-3-[tert-butoxycarbonyl-[2-(5-fluoro-3-pyridyl)-8-[2,2,2-trideuterio-1-hydroxy-1-(trideuteriomethyl)ethyl]pyrazolo[1,5-a][1,3,5]triazin-4-yl]amino]-1,2,3,4-tetrahydrocarbazole-9-carboxylate (280 mg, 168.73 μmol, 24.5% yield, 40.0% purity) as a white solid. ¹H NMR (400 MHz, CD3OD) δ ppm 9.30 (s, 1H), 8.57 (d, J=2.4 Hz, 1H), 8.42-8.36 (m, 1H), 8.31 (s, 1H), 7.95 (d, J=8.4 Hz, 1H), 7.24-7.16 (m, 1H), 7.14-7.06 (m, 1H), 7.03-6.98 (m, 1H), 4.82-4.76 (m, 1H), 3.40-3.30 (m, 2H), 3.12-3.02 (m, 2H), 2.60-2.50 (m, 1H), 2.30-2.20 (m, 1H), 1.65 (s, 9H), 1.31 (s, 9H); ES-LCMS m/z 646.3 [M−H₂O+H]⁺.

### Step 2: (3R)—N-[2-(5-Fluoro-3-pyridyl)-8-[2,2,2-trideuterio-1-(trideuteriomethyl)ethyl]pyrazolo[1,5-a][1,3,5]triazin-4-yl]-2,3,4,9-tetrahydro-1H-carbazol-3-amine (I-59)

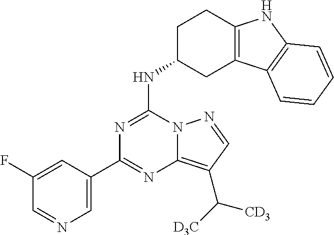 [SMILES: [2H]C([2H])([2H])C(c1cnn2c(N[C@@H]3CCc4[nH]c5ccccc5c4C3)nc(-c3cncc(F)c3)nc12)C([2H])([2H])[2H]]

To a solution of tert-butyl (3R)-3-[tert-butoxycarbonyl-[2-(5-fluoro-3-pyridyl)-8-[2,2,2-trideuterio-1-hydroxy-1-(trideuteriomethyl)ethyl]pyrazolo[1,5-a][1,3,5]triazin-4-yl]amino]-1,2,3,4-tetrahydrocarbazole-9-carboxylate (280 mg, 168.73 μmol, 1 eq) in DCM (50 mL) was added Et₃SiH (291.21 mg, 2.50 mmol, 400.01 μL, 14.84 eq), TFA (615.98 mg, 5.40 mmol, 399.99 μL, 32.02 eq) and BF₃.Et₂O (460.01 mg, 3.24 mmol, 400.01 μL, 19.21 eq). The mixture was stirred at 20° C. for 1 h. The reaction mixture was diluted with water (50 mL), basified with saturated aqueous NaHCO₃ until pH=8 and extracted with EtOAc (50 mL×3). The organic layer was dried over Na₂SO₄, filtered and concentrated under reduced pressure to yield a residue which was purified by preparative HPLC (column: Venusil ASB Phenyl 250*50 10 u; mobile phase: [water (0.05% HCl)−ACN]; B %: 70%-100%, 10 min). The desired fraction was lyophilized. To the residue was added water (10 mL). The mixture was extracted with EtOAc (10 mL×3). The organic layer was dried over Na₂SO₄, filtered and concentrated under reduced pressure to yield (3R)—N-[2-(5-fluoro-3-pyridyl)-8-[2,2,2-trideuterio-1-(trideuteriomethyl)ethyl]pyrazolo[1,5-a][1,3,5]triazin-4-yl]-2,3,4,9-tetrahydro-1H-carbazol-3-amine (29.02 mg, 63.81 μmol, 37.8% yield, 98.4% purity, [α]24.9_{D}=+10.5 (MeOH, c=0.038 g/100 mL)) as a yellow solid. ¹H NMR (400 MHz, CDCl₃) δ ppm 9.52 (s, 1H), 8.56 (d, J=2.4 Hz, 1H), 8.45 (d, J=9.6 Hz, 1H), 7.95-7.77 (m, 2H), 7.47 (d, J=7.6 Hz, 1H), 7.35 (d, J=8.0 Hz, 1H), 7.22-7.16 (m, 1H), 7.15-7.09 (m, 1H), 6.72 (d, J=8.4 Hz, 1H), 5.00-4.90 (m, 1H), 3.37 (dd, J=5.2, 15.2 Hz, 1H), 3.30-3.24 (m, 1H), 3.08-2.90 (m, 3H), 2.43-2.25 (m, 2H); ES-LCMS m/z 448.3 [M+H]⁺.

### Example 62

### Synthesis of Compound I-60a, I-60b and I-60c

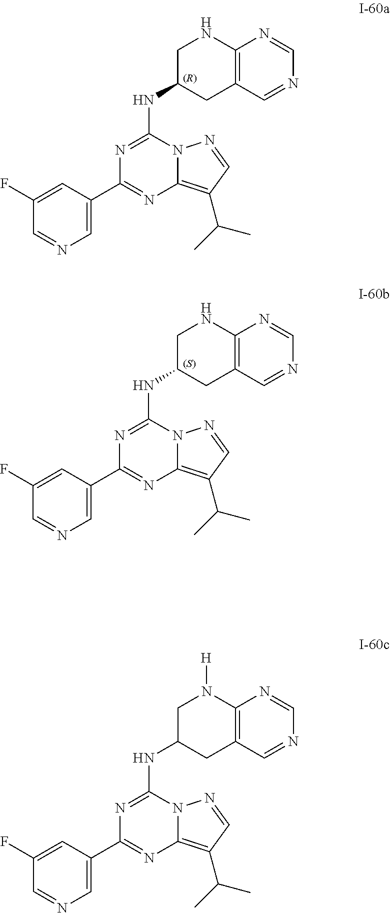

### Synthetic Scheme:

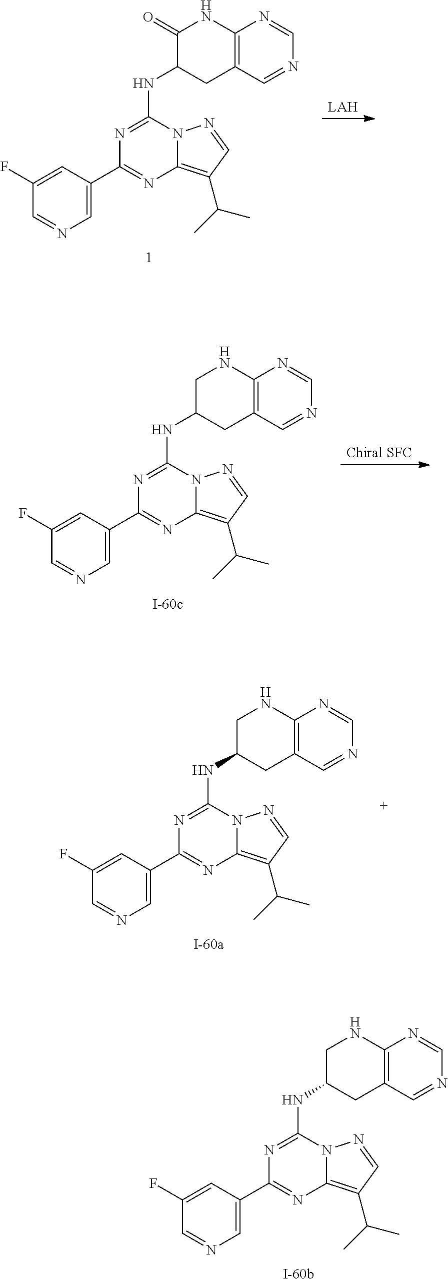

### Step 1: (6R)—N-[2-(5-Fluoro-3-pyridyl)-8-isopropyl-pyrazolo[1,5-a][1,3,5]triazin-4-yl]-5,6,7,8-tetrahydropyrido[2,3-d]pyrimidin-6-amine (I-60a) and (6S)—N-[2-(5-fluoro-3-pyridyl)-8-isopropyl-pyrazolo[1,5-a][1,3,5]triazin-4-yl]-5,6,7,8-tetrahydropyrido[2,3-d]pyrimidin-6-amine (I-60b)

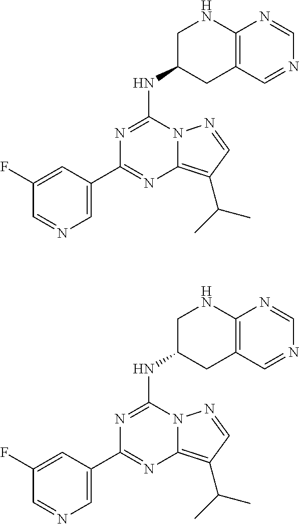 [SMILES: CC(C)c1cnn2c(N[C@H]3CNc4ncncc4C3)nc(-c3cncc(F)c3)nc12 ; CC(C)c1cnn2c(N[C@@H]3CNc4ncncc4C3)nc(-c3cncc(F)c3)nc12]

To a mixture of LiAlH₄ (450.00 mg, 11.86 mmol, 16.25 eq) in THE (50 mL) was added a solution of 6-[[2-(5-fluoro-3-pyridyl)-8-isopropyl-pyrazolo[1,5-a][1,3,5]triazin-4-yl]amino]-6,8-dihydro-5H-pyrido[2,3-d]pyrimidin-7-one (340 mg, 729.59 μmol, 1 eq) in THE (10 mL) dropwise at 0° C. The mixture was stirred at 0° C. for 1 h. The reaction mixture was quenched with water (1 mL), aqueous NaOH (3 mL, 15%), water (1 mL) and filtered. The filtrate was concentrated under reduced pressure to yield a residue which was purified by preparative HPLC (column: Xtimate C18 150*25 mm*5 um; mobile phase: [water (0.05% HCl)−ACN]; B %: 20%-50%, 8 min). The desired fraction was lyophilized and then separated by chiral SFC (column: DAICEL CHIRALPAK AD (250 mm*30 mm, 10 um); mobile phase: [0.1% NH₃.H₂O/EtOH]; B %: 45%-45%) to yield peak 1 and peak 2. One of these peaks was concentrated under reduced pressure. The residue was lyophilized to yield an enantiomer (33.13 mg, 81.32 μmol, 11.2% yield, 99.5% purity, SFC: Rₜ=2.105, ee=100%, [α]^{26.5}_{D}=−20.0 (DMSO, c=0.075 g/100 mL)) as a white solid. ¹H NMR (400 MHz, CD3OD) δ ppm 9.40 (s, 1H), 8.55 (d, J=2.8 Hz, 1H), 8.52 (dd, J=1.6, 9.6 Hz, 1H), 8.25 (s, 1H), 8.00-7.93 (m, 2H), 4.92-4.88 (m, 1H), 3.79 (dd, J=2.8, 12.4 Hz, 1H), 3.61 (dd, J=7.2, 12.4 Hz, 1H), 3.27-3.15 (m, 2H), 3.12-3.04 (m, 1H), 1.39 (d, J=7.2 Hz, 6H); ES-LCMS m/z 406.2 [M+H]⁺. The other of these peaks was concentrated under reduced pressure. The residue was lyophilized to yield the other enantiomer (43.44 mg, 104.18 μmol, 14.3% yield, 97.2% purity, SFC: Rₜ=3.503, ee=98.062%, [α]^{26.5}_{D}=+21.2 (DMSO, c=0.085 g/100 mL)) as a white solid. ¹H NMR (400 MHz, CD3OD) δ ppm 9.39 (s, 1H), 8.55 (d, J=2.8 Hz, 1H), 8.53-8.49 (m, 1H), 8.25 (s, 1H), 7.99-7.94 (m, 2H), 4.92-4.88 (m, 1H), 3.79 (dd, J=2.4, 12.4 Hz, 1H), 3.61 (dd, J=6.8, 12.4 Hz, 1H), 3.26-3.15 (m, 2H), 3.12-3.05 (m, 1H), 1.39 (d, J=6.8 Hz, 6H); ES-LCMS m/z 406.2 [M+H]⁺.

### Example 63

### Synthesis of Compound I-61a, I-61b and I-61c

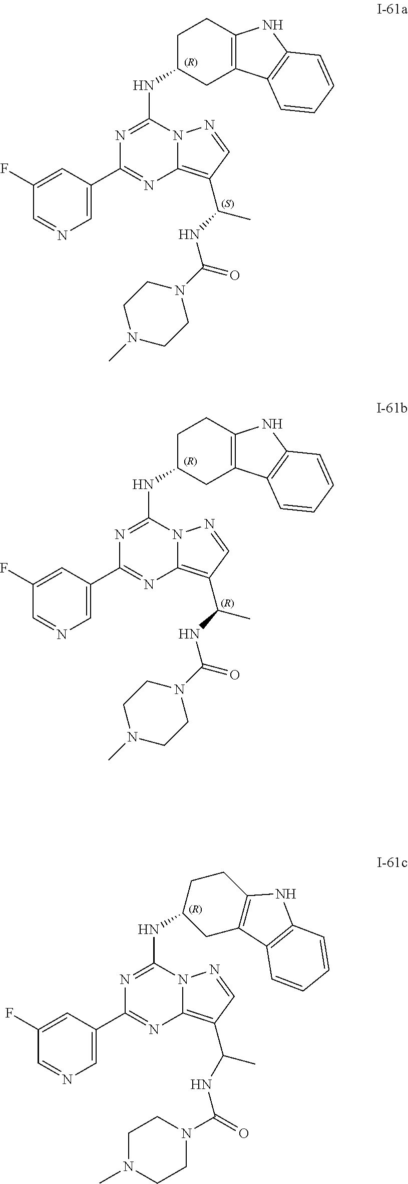

### Synthetic Scheme:

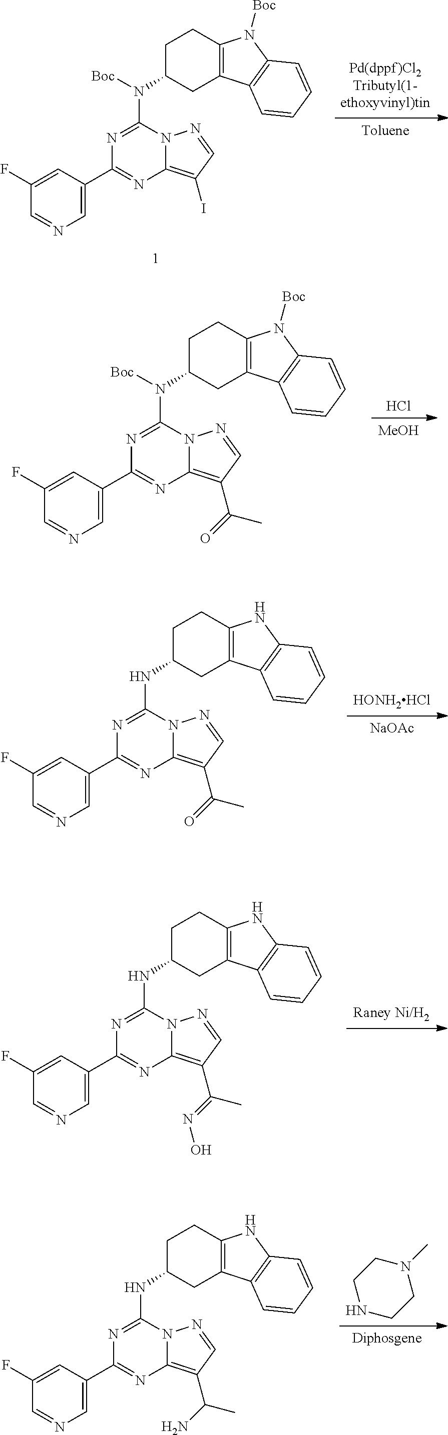

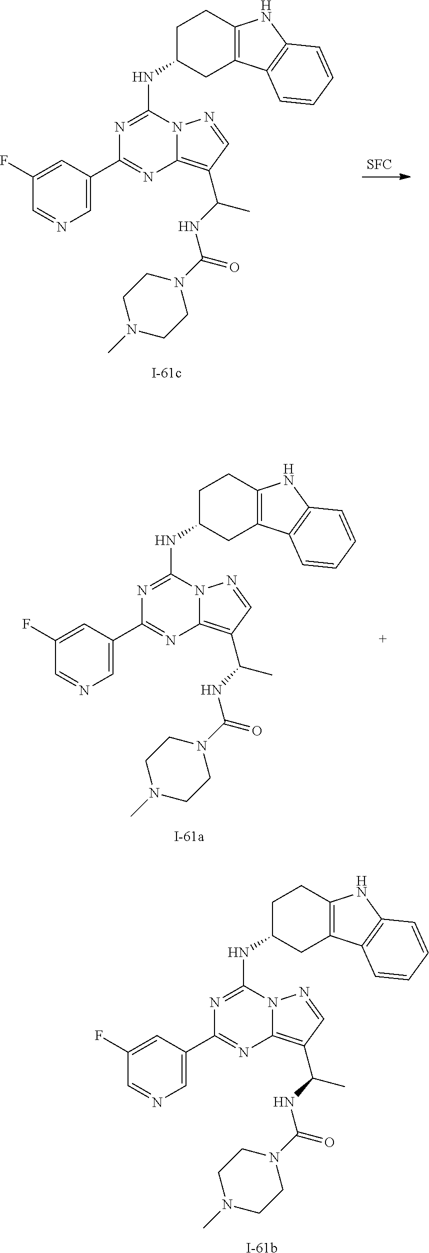

### Step 1: tert-Butyl (3R)-3-[[8-acetyl-2-(5-fluoro-3-pyridyl)pyrazolo[1,5-a][1,3,5]triazin-4-yl]-tert-butoxycarbonyl-amino]-1,2,3,4-tetrahydrocarbazole-9-carboxylate

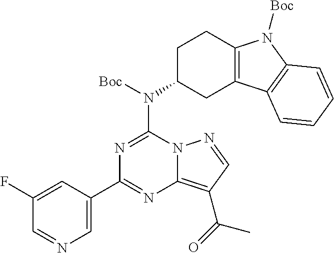 [SMILES: CC(=O)c1cnn2c(N(C(=O)OC(C)(C)C)[C@@H]3CCc4c(c5ccccc5n4C(=O)OC(C)(C)C)C3)nc(-c3cncc(F)c3)nc12]

To a solution of tert-butyl (3R)-3-[tert-butoxycarbonyl-[2-(5-fluoro-3-pyridyl)-8-iodo-pyrazolo[1,5-a][1,3,5]triazin-4-yl]amino]-1,2,3,4-tetrahydrocarbazole-9-carboxylate (2.5 g, 3.45 mmol, 1 eq), tributyl(1-ethoxyvinyl)stannane (5.66 g, 15.67 mmol, 5.29 mL, 4.55 eq) in toluene (50 mL) was added Pd(dppf)Cl₂ (252.12 mg, 344.57 μmol, 0.1 eq). The mixture was degassed and purged with N₂ for three times and stirred at 120° C. for 12 h under N₂ atmosphere. After cooled, to the mixture was added saturated KF solution (80 mL) and stirred for 10 min. The mixture was extracted with EtOAc (100 mL×3). The combined organic layers were washed with brine (100 mL), dried over Na₂SO₄, filtered and concentrated to yield a residue which was purified by flash silica gel chromatography (from PE/EtOAc=100/1 to 3/1, TLC: PE/EtOAc=3/1, R_{f}=0.50) to yield tert-butyl (3R)-3-[[8-acetyl-2-(5-fluoro-3-pyridyl)pyrazolo[1,5-a][1,3,5]triazin-4-yl]-tert-butoxycarbonyl-amino]-1,2,3,4-tetrahydrocarbazole-9-carboxylate (1.25 g, 1.95 mmol, 56.5% yield, 100.0% purity) as a yellow solid. ¹H NMR (400 MHz, DMSO-d₆) δ ppm 9.42 (s, 1H), 8.87 (s, 1H), 8.83 (d, J=2.7 Hz, 1H), 8.54-8.48 (m, 1H), 8.00 (d, J=8.3 Hz, 1H), 7.41 (d, J=7.6 Hz, 1H), 7.25-7.19 (m, 1H), 7.17-7.11 (m, 1H), 4.81 (s, 1H), 3.33-3.19 (m, 3H), 3.16-3.00 (m, 2H), 2.80 (s, 3H), 2.27 (s, 1H), 1.60 (s, 9H), 1.31 (s, 9H); ES-LCMS m/z 642.3 [M+H]⁺.

### Step 2: 1-[2-(5-Fluoro-3-pyridyl)-4-[[(3R)-2,3,4,9-tetrahydro-1H-carbazol-3-yl]amino]pyrazolo[1,5-a][1,3,5]triazin-8-yl]ethanone

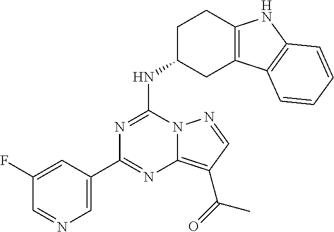 [SMILES: CC(=O)c1cnn2c(N[C@@H]3CCc4[nH]c5ccccc5c4C3)nc(-c3cncc(F)c3)nc12]

To a stirred solution of tert-butyl (3R)-3-[[8-acetyl-2-(5-fluoro-3-pyridyl)pyrazolo[1,5-a][1,3,5]triazin-4-yl]-tert-butoxycarbonyl-amino]-1,2,3,4-tetrahydrocarbazole-9-carboxylate (1.25 g, 1.95 mmol, 1 eq) in MeOH (40 mL) was added HCl (12 M, 30 mL, 184.81 eq). The reaction mixture was stirred at 40° C. for 3.5 h. The reaction mixture was diluted with water (50 mL) then adjusted pH to 8-9 by saturated NaHCO₃ solution, extracted with EtOAc (100 mL×3). The combined organic layers were dried over anhydrous Na₂SO₄ then filtered. The filtrate was concentrated to yield 1-[2-(5-fluoro-3-pyridyl)-4-[[(3R)-2,3,4,9-tetrahydro-1H-carbazol-3-yl]amino]pyrazolo[1,5-a][1,3,5]triazin-8-yl]ethanone (800 mg, 1.81 mmol, 93.0% yield, 100.0% purity) as a yellow solid which was used in the next step without further purification. ¹H NMR (400 MHz, DMSO-d₆) δ ppm 10.80 (s, 1H), 9.54-9.40 (m, 2H), 8.77 (d, J=2.9 Hz, 1H), 8.61-8.49 (m, 2H), 7.34 (d, J=7.6 Hz, 1H), 7.27 (d, J=8.1 Hz, 1H), 7.04-6.98 (m, 1H), 6.96-6.89 (m, 1H), 4.87 (s, 1H), 3.15-3.09 (m, 1H), 3.07-2.99 (m, 1H), 2.99-2.86 (m, 2H), 2.74 (s, 3H), 2.21 (d, J=3.4 Hz, 2H); ES-LCMS m/z 442.2 [M+H]⁺.

### Step 3: 1-[2-(5-Fluoro-3-pyridyl)-4-[[(3R)-2,3,4,9-tetrahydro-1H-carbazol-3-yl]amino]pyrazolo[1,5-a][1,3,5]triazin-8-yl]ethanone oxime

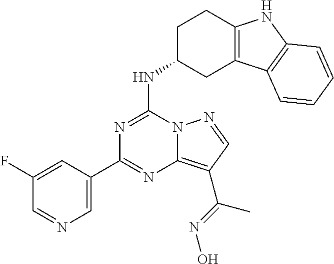 [SMILES: C/C(=N\O)c1cnn2c(N[C@@H]3CCc4[nH]c5ccccc5c4C3)nc(-c3cncc(F)c3)nc12]

To a stirred solution of 1-[2-(5-fluoro-3-pyridyl)-4-[[(3R)-2,3,4,9-tetrahydro-1H-carbazol-3-yl]amino]pyrazolo[1,5-a][1,3,5]triazin-8-yl]ethanone (800 mg, 1.81 mmol, 1 eq) in THE (15 mL) was added NH₂OH HCl (251.86 mg, 3.62 mmol, 2 eq) and NaOAc (445.96 mg, 5.44 mmol, 3 eq). The reaction mixture was stirred at 60° C. for 12 h. The mixture was added water (50 mL), extracted with EtOAc (50 mL×3). The combined organic layers were washed with brine (50 mL), dried over Na₂SO₄, filtered and concentrated to yield a residue which was purified by flash silica gel chromatography (from PE/EtOAc=100/1 to 1/1, TLC: PE/EtOAc=1/1, R_{f}=0.25) to yield 1-[2-(5-fluoro-3-pyridyl)-4-[[(3R)-2,3,4,9-tetrahydro-1H-carbazol-3-yl]amino]pyrazolo [1,5-a][1,3,5]triazin-8-yl]ethanone oxime (800 mg, 1.75 mmol, 96.7% yield, 100.0% purity) as a yellow solid. ¹H NMR (400 MHz, CDCl₃) δ ppm 9.60-9.50 (m, 1H), 8.66-8.58 (m, 2H), 7.93 (s, 1H), 7.47 (d, J=7.8 Hz, 1H), 7.34 (d, J=8.0 Hz, 1H), 7.19 (t, J=7.4 Hz, 1H), 7.14-7.09 (m, 1H), 6.97-6.84 (m, 1H), 4.97 (s, 1H), 3.38 (dd, J=5.0, 15.3 Hz, 1H), 3.08-2.91 (m, 3H), 2.68-2.54 (m, 3H), 2.43-2.28 (m, 2H); ES-LCMS m/z 457.2 [M+H]⁺.

### Step 4: (3R)—N-[8-(1-Aminoethyl)-2-(5-fluoro-3-pyridyl)pyrazolo[1,5-a][1,3,5]triazin-4-yl]-2,3,4,9-tetrahydro-1H-carbazol-3-amine

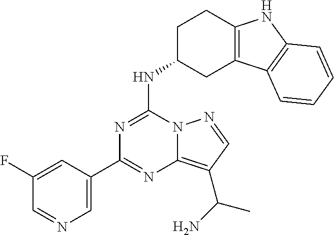 [SMILES: CC(N)c1cnn2c(N[C@@H]3CCc4[nH]c5ccccc5c4C3)nc(-c3cncc(F)c3)nc12]

To a stirred solution of 1-[2-(5-fluoro-3-pyridyl)-4-[[(3R)-2,3,4,9-tetrahydro-1H-carbazol-3-yl]amino]pyrazolo[1,5-a][1,3,5]triazin-8-yl]ethanone oxime (250 mg, 547.68 μmol, 1 eq) in a solution of THF (6 mL) and MeOH (6 mL) was added Raney-Ni (558.66 mg, 6.52 mmol, 11.91 eq) (washed by anhydrous MeOH for three times to reduce water) and a solution of NH₃ (7 M, 391.20 μL, 5 eq) in MeOH under N₂ atmosphere. The reaction mixture was degassed and purged with H₂ for three times and stirred at 30° C. for 12h under H₂ atmosphere. The reaction mixture was filtered through a pad of celite and the filtrate was concentrated to yield a residue was purified by flash silica gel chromatography (from DCM/MeOH=100/1 to 10/1, TLC: DCM/MeOH=10/1, R_{f}=0.25) to yield (3R)—N-[8-(1-aminoethyl)-2-(5-fluoro-3-pyridyl)pyrazolo[1,5-a][1,3,5]triazin-4-yl]-2,3,4,9-tetrahydro-1H-carbazol-3-amine (130 μmg, 281.45 umol, 51.4% yield, 95.8% purity) as a yellow solid. ¹H NMR (400 MHz, CD3OD) δ ppm 9.47 (s, 1H), 8.67-8.57 (m, 2H), 8.20 (s, 1H), 7.38 (d, J=7.6 Hz, 1H), 7.28 (d, J=8.1 Hz, 1H), 7.05 (t, J=7.5 Hz, 1H), 6.99-6.94 (m, 1H), 4.90-4.81 (m, 2H), 3.28 (s, 1H), 3.13-2.92 (m, 3H), 2.45-2.36 (m, 1H), 2.34-2.22 (m, 1H), 1.81 (d, J=6.8 Hz, 3H); ES-LCMS m/z 426.2 [M−NH₂]⁺.

### Step 5: N-[(1S)-1-[2-(5-Fluoro-3-pyridyl)-4-[[(3R)-2,3,4,9-tetrahydro-1H-carbazol-3-yl]amino]pyrazolo[1,5-a][1,3,5]triazin-8-yl]ethyl]-4-methyl-piperazine-1-carboxamide (I-61a) and N-[(1R)-1-[2-(5-Fluoro-3-pyridyl)-4-[[(3R)-2,3,4,9-tetrahydro-1H-carbazol-3-yl]amino]pyrazolo[1,5-a][1,3,5]triazin-8-yl]ethyl]-4-methyl-piperazine-1-carboxamide (I-61b)

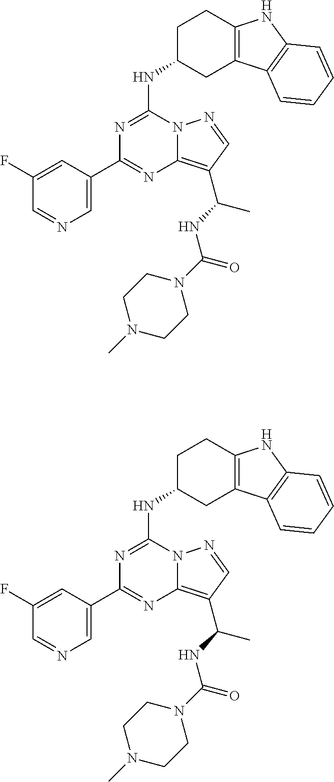 [SMILES: C[C@H](NC(=O)N1CCN(C)CC1)c1cnn2c(N[C@@H]3CCc4[nH]c5ccccc5c4C3)nc(-c3cncc(F)c3)nc12 ; C[C@@H](NC(=O)N1CCN(C)CC1)c1cnn2c(N[C@@H]3CCc4[nH]c5ccccc5c4C3)nc(-c3cncc(F)c3)nc12]

To a stirred solution of (3R)—N-[8-(1-aminoethyl)-2-(5-fluoro-3-pyridyl)pyrazolo[1,5-a][1,3,5]triazin-4-yl]-2,3,4,9-tetrahydro-1H-carbazol-3-amine (130 mg, 281.45 μmol, 1 eq) in THE (6 mL) was added trichloromethyl carbonochloridate (334.08 mg, 1.69 mmol, 203.71 μL, 6 eq). The reaction mixture was stirred at 60° C. for 2 h. 1-Methylpiperazine (903.00 mg, 9.02 mmol, 1 mL, 32.03 eq) was added to the above reaction mixture and stirred at 60° C. for 12 h. The reaction mixture was concentrated to yield a residue which was purified by preparative HPLC (basic condition; column: Boston Green ODS 150*30 5 u; mobile phase: [water (0.04% NH₃H₂O+10 mM NH₄HCO₃)−ACN]; B %: 40%-70%, 8 min) to yield the product which was separated by SFC (basic condition; column: DAICEL CHIRALPAK AS-H (250 mm*30 mm, 5 um); mobile phase: [0.1% NH₃H₂O ETOH]; B %: 40%-40%, min) to yield an enantiomer (59 mg, 103.76 μmol, 36.9% yield, 100.0% purity, SFC: Rₜ=3.560 min, ee=99.7%, [α]^{24.0}_{D}=+30.0, MeOH, c=0.02 g/100 mL) as a white solid; ¹H NMR (400 MHz, CD3OD) δ ppm 9.42 (s, 1H), 8.61-8.51 (m, 2H), 8.04 (s, 1H), 7.38 (d, J=7.8 Hz, 1H), 7.27 (d, J=8.0 Hz, 1H), 7.06-7.01 (m, 1H), 6.99-6.93 (m, 1H), 5.29 (q, J=6.9 Hz, 1H), 4.86 (s, 1H), 3.54-3.41 (m, 4H), 3.28 (d, J=5.3 Hz, 1H), 3.11-2.89 (m, 3H), 2.49-2.35 (m, 5H), 2.32-2.20 (m, 4H), 1.66 (d, J=6.8 Hz, 3H); ES-LCMS m/z 569.3 [M+H]⁺ and the other enantiomer (19.3 mg, 33.94 μmol, 12.1% yield, 100.0% purity, SFC: Rₜ=3.990 min, ee=99.1%, [α]^{23.9}_{D}=+20.0, MeOH, c=0.02 g/100 mL) as a white solid; ¹H NMR (400 MHz, CD3OD) δ ppm 9.42 (s, 1H), 8.60-8.52 (m, 2H), 8.03 (s, 1H), 7.38 (d, J=7.5 Hz, 1H), 7.27 (d, J=8.0 Hz, 1H), 7.06-7.01 (m, 1H), 6.98-6.93 (m, 1H), 5.29 (q, J=6.9 Hz, 1H), 4.87 (s, 1H), 3.56-3.42 (m, 4H), 3.28 (d, J=5.3 Hz, 1H), 3.11-2.88 (m, 3H), 2.47 (t, J=4.9 Hz, 4H), 2.37 (s, 1H), 2.32 (s, 3H), 2.30-2.20 (m, 1H), 1.66 (d, J=7.0 Hz, 3H); ES-LCMS m/z 569.3 [M+H]⁺.

### Example 64

### Synthesis of Compound I-62a, I-62b and I-62c

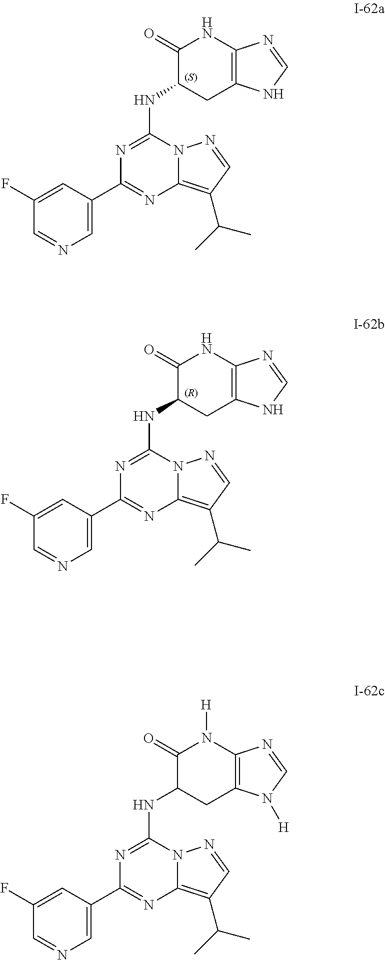

### Synthetic Scheme:

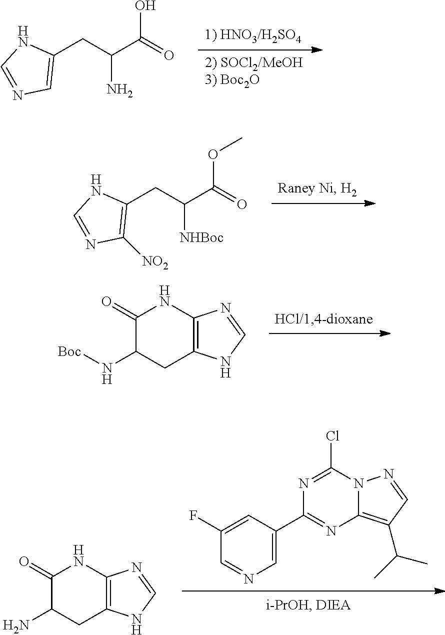

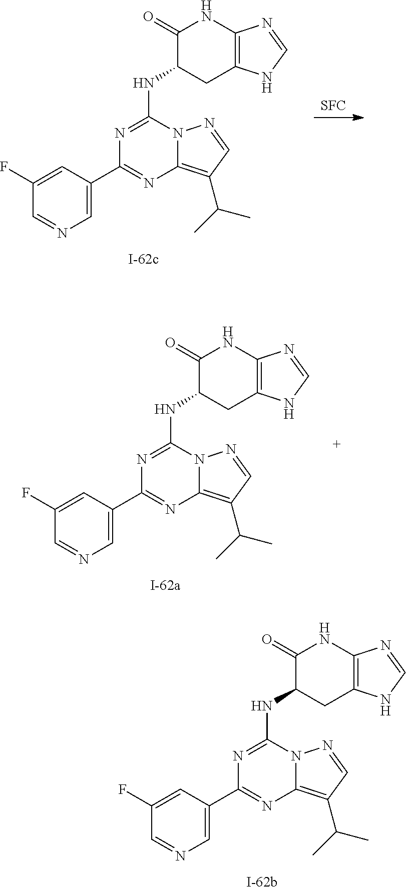

### Step 1: Methyl 2-(tert-butoxycarbonylamino)-3-(4-nitro-1H-imidazol-5-yl)propanoate

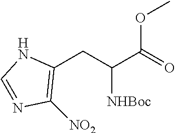 [SMILES: COC(=O)C(Cc1[nH]cnc1[N+](=O)[O-])NC(=O)OC(C)(C)C]

To a solution of 2-amino-3-(1H-imidazol-5-yl)propanoic acid (2 g, 12.89 mmol, 1 eq) in conc. H₂SO₄ (12 mL) was added nitric acid (5.49 g, 87.14 mmol, 3.92 mL, 6.76 eq) at 0° C. The mixture was stirred at 25° C. for 1 h, then was added to MeOH (80 mL) dropwise. After being stirred at 60° C. for 3 h, the mixture was poured into cold H₂O (240 mL), neutralized with NaOH (solid) to pH=7-8 at 0° C., concentrated to remove MeOH. To the aqueous residue was added THE (80 mL), Na₂CO₃ (4.10 g, 38.67 mmol, 3 eq) and tert-butoxycarbonyl tert-butyl carbonate (2.81 g, 12.89 mmol, 2.96 mL, 1.0 eq). The resulting mixture was stirred at 25° C. for 12 h. The reaction mixture was extracted with EtOAc (100 mL×3). The combined organic layers were washed with brine (50 mL), dried over Na₂SO₄, filtered and concentrated under reduced pressure to yield a residue which was purified by flash silica gel chromatography (from PE/EtOAc=1/0 to 0/1, TLC: EtOAc, R_{f}=0.50) to yield methyl 2-(tert-butoxycarbonylamino)-3-(4-nitro-1H-imidazol-5-yl)propanoate (1.5 g, 4.61 mmol, 35.7% yield, 96.6% purity) as a white solid. ¹H NMR (400 MHz, DMSO-d₆) δ ppm 13.01 (br s, 1H), 7.73 (s, 1H), 7.35 (d, J=8.1 Hz, 1H), 4.42-4.33 (m, 1H), 3.59 (s, 3H), 3.43 (dd, J=6.6, 14.2 Hz, 1H), 3.29-3.20 (m, 1H), 1.33 (s, 9H); ES-LCMS m/z 259.1 [M−t-Bu+H]⁺.

### Step 2: tert-Butyl N-(5-oxo-1,4,6,7-tetrahydroimidazo[4,5-b]pyridin-6-yl)carbamate

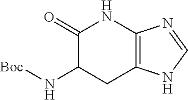 [SMILES: CC(C)(C)OC(=O)NC1Cc2[nH]cnc2NC1=O]

To a solution of methyl 2-(tert-butoxycarbonylamino)-3-(4-nitro-1H-imidazol-5-yl)propanoate (800 mg, 2.46 mmol, 1 eq) in MeOH (100 mL) was added Raney-Ni (1.0 g, 11.67 mmol, 4.75 eq) under H₂ (35 psi). The mixture was stirred at 60° C. for 40 h. The mixture was filtered through celite, then the cake was rinsed with MeOH (100 mL×2). The filtrate was concentrated to yield a residue (500 mg crude) which was purified by preparative HPLC (column: Boston Green ODS 150*30 5 μm; mobile phase: [water (0.04% NH₃.H₂O+10 mM NH₄HCO₃)-ACN]; B %: 15%-45%, 8 min), followed by lyophilization to yield tert-butyl N-(5-oxo-1,4,6,7-tetrahydroimidazo[4,5-b]pyridin-6-yl)carbamate (130 mg, 513.78 μmol, 20.9% yield, 99.7% purity) as a white solid. ¹H NMR (400 MHz, DMSO-d₆) δ ppm 11.84 (br s, 1H), 9.98 (br s, 1H), 7.29 (s, 1H), 6.91 (d, J=8.3 Hz, 1H), 4.31-4.08 (m, 1H), 2.97 (dd, J=8.1, 15.2 Hz, 1H), 2.78-2.68 (m, 1H), 1.40 (s, 9H); ES-LCMS m/z 253.2 [M+H]⁺.

### Step 3: 6-Amino-1,4,6,7-tetrahydroimidazo[4,5-b]pyridin-5-one

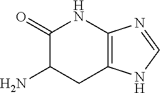 [SMILES: NC1Cc2[nH]cnc2NC1=O]

To a solution of tert-butyl N-(5-oxo-1,4,6,7-tetrahydroimidazo[4,5-b]pyridin-6-yl)carbamate (110 mg, 434.73 μmol, 1 eq) in THF (10 mL) was added HCl/1,4-dioxane (4 M, 2 mL, 18.40 eq). The mixture was stirred at 15° C. for 12 h. The reaction mixture was concentrated to yield 6-amino-1,4,6,7-tetrahydroimidazo[4,5-b]pyridin-5-one (97.8 mg, crude, 2HCl) as a white solid, which was used in the next step without further purification. H NMR (400 MHz, DMSO-d₆) δ ppm 11.38 (s, 1H), 8.78 (br s, 3H), 8.48 (s, 1H), 4.46-4.36 (m, 1H), 3.41-3.35 (m, 1H), 3.12-3.00 (m, 1H); ES-LCMS m/z 153.2 [M+H]⁺.

### Step 4: 6-[[2-(5-Fluoro-3-pyridyl)-8-isopropyl-pyrazolo[1,5-a][1,3,5]triazin-4-yl]amino]-1,4,6,7-tetrahydroimidazo[4,5-b]pyridin-5-one

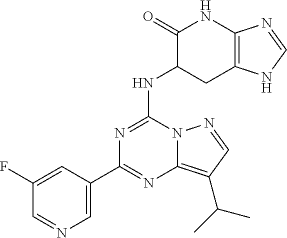 [SMILES: CC(C)c1cnn2c(NC3Cc4[nH]cnc4NC3=O)nc(-c3cncc(F)c3)nc12]

To a solution of 6-amino-1,4,6,7-tetrahydroimidazo[4,5-b]pyridin-5-one (97.8 mg, 434.52 μmol, 1 eq, 2HCl) in i-PrOH (10 mL) was added DIEA (168.47 mg, 1.30 mmol, 227.05 μL, 3 eq) and 4-chloro-2-(5-fluoro-3-pyridyl)-8-isopropyl-pyrazolo[1,5-a][1,3,5]triazine (126.75 mg, 434.52 μmol, 1 eq). The mixture was stirred at 50° C. for 1 h. The reaction mixture was concentrated to yield a residue which was purified by flash silica gel chromatography (from EtOAc/MeOH=1/0 to 10/1, TLC: EtOAc/MeOH=10/1, R_{f}=0.49) to yield 6-[[2-(5-fluoro-3-pyridyl)-8-isopropyl-pyrazolo[1,5-a][1,3,5]triazin-4-yl]amino]-1,4,6,7-tetrahydroimidazo[4,5-b]pyridin-5-one (150 mg, 360.09 μmol, 82.8% yield, 97.8% purity) as a white solid. ¹H NMR (400 MHz, DMSO-d₆) δ ppm 11.95 (br s, 1H), 10.25 (s, 1H), 9.35 (s, 1H), 8.97 (d, J=8.6 Hz, 1H), 8.69 (d, J=2.9 Hz, 1H), 8.46 (d, J=10.0 Hz, 1H), 8.18 (s, 1H), 7.37 (s, 1H), 5.46-5.29 (m, 1H), 3.29-3.26 (m, 2H), 3.23-3.15 (m, 1H), 1.37 (d, J=6.8 Hz, 6H); ES-LCMS m/z 408.2 [M+H]⁺.

### Step 5: (6S)-6-[[2-(5-Fluoro-3-pyridyl)-8-isopropyl-pyrazolo[1,5-a][1,3,5]triazin-4-yl]amino]-1,4,6,7-tetrahydroimidazo[4,5-b]pyridin-5-one (I-62a) and (6R)-6-[[2-(5-fluoro-3-pyridyl)-8-isopropyl-pyrazolo[1,5-a][1,3,5]triazin-4-yl]amino]-1,4,6,7-tetrahydroimidazo [4,5-b]pyridin-5-one (I-62b)

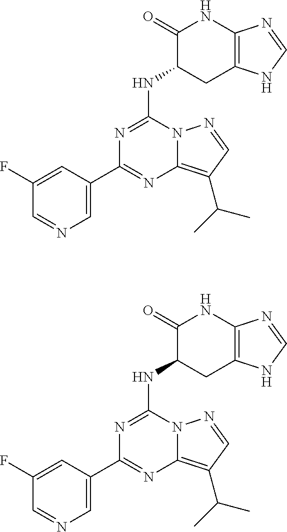 [SMILES: CC(C)c1cnn2c(N[C@H]3Cc4[nH]cnc4NC3=O)nc(-c3cncc(F)c3)nc12 ; CC(C)c1cnn2c(N[C@@H]3Cc4[nH]cnc4NC3=O)nc(-c3cncc(F)c3)nc12]

6-[[2-(5-Fluoro-3-pyridyl)-8-isopropyl-pyrazolo[1,5-a][1,3,5]triazin-4-yl]amino]-1,4,6,7-tetrahydroimidazo[4,5-b]pyridin-5-one (100 mg, 240.06 μmol, 1 eq) was separated by SFC (column: YMC CHIRAL Amylose-C (250 mm*30 mm, 10 μm; mobile phase: [0.1% NH₃.H₂O EtOH]; B %: 45%-45%, Peak 1=2.726; Peak 2=3.912) to yield peak 1 and peak 2. One of these peaks was concentrated to yield a residue which was added MeCN (30 mL) and water (30 mL), followed by lyophilization to yield an enantiomer (17.4 mg, 41.98 μmol, 17.4% yield, 98.3% purity, SFC: Rₜ=2.721, ee=99.44%, [α]^{27.1}_{D}=+69.23 (MeOH, c=0.026 g/100 mL)) as a yellow solid. ¹H NMR (400 MHz, CD3OD) δ ppm 9.37 (s, 1H), 8.55 (d, J=2.7 Hz, 1H), 8.49 (dd, J=1.6, 9.7 Hz, 1H), 8.04 (s, 1H), 7.45 (s, 1H), 5.33 (dd, J=8.3, 13.2 Hz, 1H), 3.60 (dd, J=8.4, 15.3 Hz, 1H), 3.30-3.20 (m, 2H), 1.43 (d, J=7.1 Hz, 6H); ES-LCMS m/z 408.2 [M+H]⁺. The other of these peaks was concentrated to yield a residue which was added MeCN (30 mL) and water (30 mL), followed by lyophilization to yield the other enantiomer (16.1 mg, 39.44 μmol, 16.4% yield, 99.8% purity, SFC: Rₜ=3.908, ee=98.6%, [α]^{27.2}_{D}=−63.64 (MeOH, c=0.022 g/100 mL)) as a yellow solid. ¹H NMR (400 MHz, CD3OD) δ ppm 9.35 (s, 1H), 8.53 (d, J=2.7 Hz, 1H), 8.49-8.43 (m, 1H), 8.02 (s, 1H), 7.43 (s, 1H), 5.31 (dd, J=8.6, 13.2 Hz, 1H), 3.58 (dd, J=8.3, 14.9 Hz, 1H), 3.28-3.18 (m, 2H), 1.41 (d, J=7.1 Hz, 6H); ES-LCMS m/z 408.2 [M+H]⁺.

### Example 65

### Synthesis of Compound I-63a, I-63b and I-63c

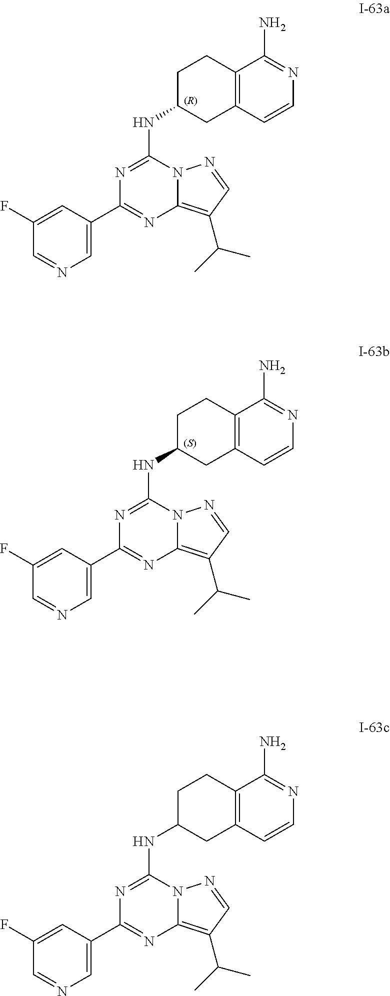

### Synthetic Scheme:

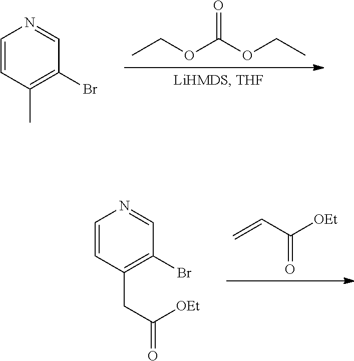

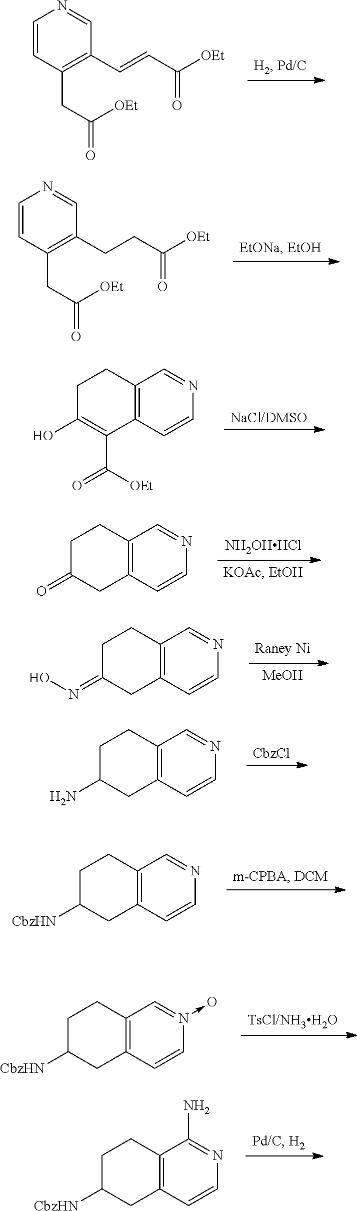

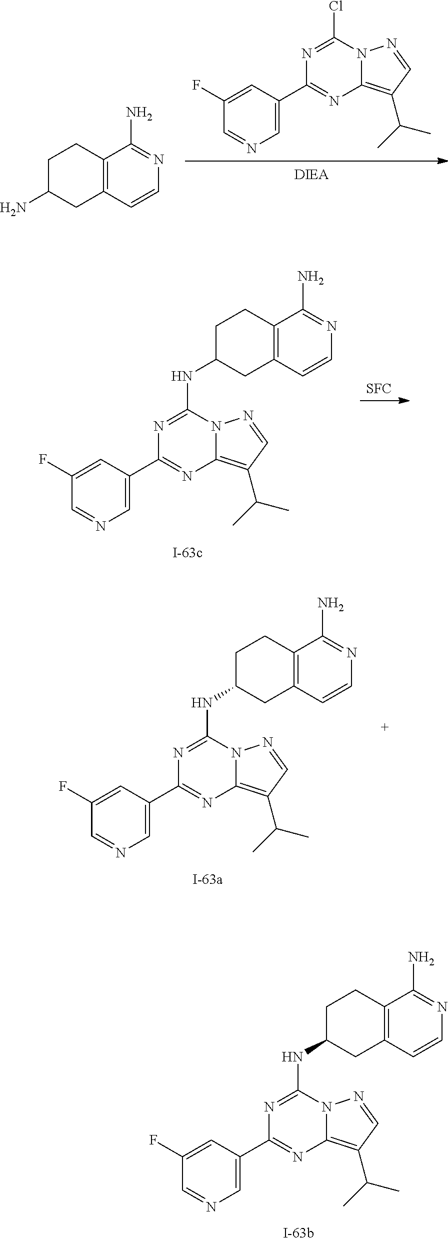

### Step 1: Ethyl 2-(3-bromo-4-pyridyl)acetate

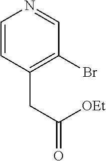 [SMILES: CCOC(=O)Cc1ccncc1Br]

To a solution of 3-bromo-4-methyl-pyridine (15.50 g, 90.10 mmol, 10 mL, 1 eq) and diethyl carbonate (12.59 g, 106.61 mmol, 12.92 mL, 1.18 eq) in THE (150 mL) was added LiHMDS (1 M, 142.08 mL, 1.58 eq) dropwise under N₂ atmosphere at 0° C. The mixture was stirred under N₂ atmosphere at 0° C. for 3 h. TLC (PE/EtOAc=5/1, R_{f}=0.3) showed the starting material was consumed completely. The reaction mixture was quenched with saturated aq. NH₄C1 solution (100 mL) slowly and extracted with EtOAc (100 mL×3). The organic layer was dried over Na₂SO₄, filtered and concentrated under reduced pressure to yield a residue which was purified by flash silica gel chromatography (from PE/EtOAc=0/1 to 5/1, TLC: PE/EtOAc=5/1, R_{f}=0.3) to yield ethyl 2-(3-bromo-4-pyridyl)acetate (15 g, 61.45 mmol, 68.2% yield) as brown oil. ¹H NMR (400 MHz, CDCl₃) δ ppm 8.73 (s, 1H), 8.49 (d, J=4.8 Hz, 1H), 7.28 (d, J=4.8 Hz, 1H), 4.21 (q, J=6.8 Hz, 2H), 3.79 (s, 2H), 1.29 (t, J=6.8 Hz, 3H); ES-LCMS m/z 244.0, 246.0 [M+H]⁺.

### Step 2: Ethyl (E)-3-[4-(2-ethoxy-2-oxo-ethyl)-3-pyridyl]prop-2-enoate

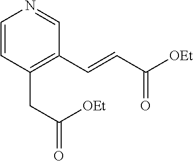 [SMILES: CCOC(=O)/C=C/c1cnccc1CC(=O)OCC]

A mixture of ethyl 2-(3-bromo-4-pyridyl)acetate (17 g, 69.65 mmol, 1 eq), ethyl prop-2-enoate (35 g, 349.60 mmol, 38.00 mL, 5.02 eq), Pd(OAc)₂ (781.83 mg, 3.48 mmol, 0.05 eq), tri-o-tolylphosphine (2.12 g, 6.96 mmol, 0.1 eq) and Et₃N (109.05 g, 1.08 mol, 150 mL, 15.47 eq) was stirred under N₂ atmosphere at 95° C. for 12 h. The reaction mixture was concentrated under reduced pressure to yield a residue which was purified by flash silica gel chromatography (from PE/EtOAc=20/1 to 1/1, TLC: PE/EtOAc=1/1, R_{f}=0.30) to yield ethyl (E)-3-[4-(2-ethoxy-2-oxo-ethyl)-3-pyridyl]prop-2-enoate (5.7 g, 5.41 mmol, 7.77% yield, 25% purity) as colorless oil. H NMR (400 MHz, CDCl₃) δ ppm 8.79 (s, 1H), 8.55 (d, J=5.2 Hz, 2H), 7.88 (d, J=16.0 Hz, 1H), 6.46 (d, J=16.0 Hz, 1H), 4.29 (q, J=7.2 Hz, 2H), 4.12 (q, J=7.2 Hz, 2H), 3.76 (s, 2H), 1.35 (t, J=7.2 Hz, 3H), 1.27 (t, J=7.2 Hz, 3H); ES-LCMS m/z 264.1 [M+H]⁺.

### Step 3: Ethyl 3-[4-(2-ethoxy-2-oxo-ethyl)-3-pyridyl]propanoate

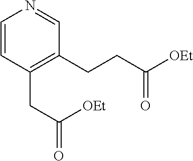 [SMILES: CCOC(=O)CCc1cnccc1CC(=O)OCC]

A mixture of ethyl (E)-3-[4-(2-ethoxy-2-oxo-ethyl)-3-pyridyl]prop-2-enoate (9 g, 10.25 mmol, 1 eq) and Pd/C (1 g, 10% purity) in EtOH (50 mL) and EtOAc (50 mL) was stirred under H₂ (30 Psi) at 30° C. for 12 h. The mixture was filtered. The filtrate was concentrated under reduced pressure to yield a residue which was purified by flash silica gel chromatography (from PE/EtOAc=20/1 to 5/1, TLC: PE/EtOAc=1/1, R_{f}=0.40) to yield ethyl 3-[4-(2-ethoxy-2-oxo-ethyl)-3-pyridyl]propanoate (2.7 g, 10.04 mmol, 98.0% yield, 98.7% purity) as colorless oil. ¹H NMR (400 MHz, CDCl₃) δ ppm 8.46 (s, 1H), 8.42 (d, J=5.2 Hz, 1H), 7.16 (d, J=4.8 Hz, 1H), 4.16 (dd, J=7.2, 14.4 Hz, 4H), 3.69 (s, 2H), 3.00 (t, J=7.6 Hz, 2H), 2.63 (t, J=7.6 Hz, 2H), 1.27-1.23 (m, 6H); ES-LCMS m/z 266.2 [M+H]⁺.

### Step 4: Ethyl 6-hydroxy-7,8-dihydroisoquinoline-5-carboxylate

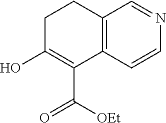 [SMILES: CCOC(=O)C1=C(O)CCc2cnccc21]

To EtOH (20 mL) was added Na (1.15 g, 50.22 mmol, 1.19 mL, 5 eq) at 20° C. The mixture was stirred at 20° C. until Na disappeared. To a mixture of ethyl 3-[4-(2-ethoxy-2-oxo-ethyl)-3-pyridyl]propanoate (2.7 g, 10.04 mmol, 1 eq) in EtOH (100 mL) was added above NaOMe solution at 20° C. The mixture was stirred at 90° C. for 1 h. TLC (PE/EtOAc=1/1, R_{f}=0.20) showed the starting material was consumed completely. The reaction mixture was cooled to 20° C., poured into aqueous HCl (30 mL, 2M), basified with saturated aqueous NaHCO₃ until pH=8 and concentrated under reduced pressure. The residue was diluted with water (20 mL) and extracted with DCM (50 mL×3). The organic layer was dried over Na₂SO₄, filtered and concentrated under reduced pressure to yield ethyl 6-hydroxy-7,8-dihydroisoquinoline-5-carboxylate (1.2 g, 5.47 mmol, 54.5% yield, 100.0% purity) as a white solid, which was used in the next step without further purification. ¹H NMR (400 MHz, CDCl₃) δ ppm 13.81 (s, 1H), 8.38 (d, J=5.6 Hz, 1H), 8.32 (s, 1H), 7.64 (d, J=5.6 Hz, 1H), 4.43 (q, J=7.2 Hz, 2H), 2.87-2.80 (m, 2H), 2.66-2.58 (m, 2H), 1.44 (t, J=7.2 Hz, 3H); ES-LCMS m/z 220.1 [M+H]⁺.

### Step 5: 7,8-Dihydro-5H-isoquinolin-6-one

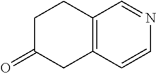 [SMILES: O=C1CCc2cnccc2C1]

To a solution of HCl (12 M, 15 mL, 32.89 eq) in H₂O (120 mL) was added ethyl 6-oxo-7,8-dihydro-5H-isoquinoline-5-carboxylate (1.2 g, 5.47 mmol, 1 eq). The mixture was stirred at 120° C. for 4 h. The reaction mixture was concentrated under reduced pressure to yield 7,8-dihydro-5H-isoquinolin-6-one (1 g, 5.45 mmol, 99.5% yield, N/A purity, HCl) as a brown solid, which was used in the next step without further purification. ¹H NMR (400 MHz, CD3OD) δ ppm 8.61 (s, 1H), 8.52 (d, J=6.0 Hz, 1H), 8.21-8.16 (m, 3H), 7.80 (d, J=6.0 Hz, 1H), 7.26 (d, J=6.4 Hz, 2H), 3.04 (s, 2H), 2.59 (t, J=8.4 Hz, 3H), 2.11 (t, J=6.8 Hz, 2H); ES-LCMS m/z 148.1 [M+H]⁺.

### Step 6: 7,8-Dihydro-5H-isoquinolin-6-one oxime

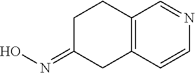 [SMILES: O/N=C1\CCc2cnccc2C1]

A mixture of 7,8-dihydro-5H-isoquinolin-6-one (1 g, 5.45 mmol, 1 eq, HCl), NH₂OH HCl (600 mg, 8.63 mmol, 1.59 eq) and KOAc (2.67 g, 27.23 mmol, 5 eq) in EtOH (50 mL) was stirred at 20° C. for 2 h. The reaction mixture was concentrated under reduced pressure to yield 7,8-dihydro-5H-isoquinolin-6-one oxime (4 g, crude) as an off-white solid, which was used in the next step without further purification. ES-LCMS m/z 163.1 [M+H]⁺.

### Step 7: 5,6,7,8-Tetrahydroisoquinolin-6-amine

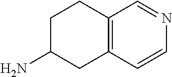 [SMILES: NC1CCc2cnccc2C1]

A mixture of 7,8-dihydro-5H-isoquinolin-6-one oxime (4 g, 24.66 mmol, 1 eq), Raney-Ni (500 mg) and NH₃/MeOH (7 M, 5 mL) in MeOH (100 mL) was stirred under H₂ (15 Psi) at 45° C. for 12 h. The reaction mixture was filtered. The filtrate was concentrated under reduced pressure to yield 5,6,7,8-tetrahydroisoquinolin-6-amine (3.5 g, crude) as an off-white solid, which was used in the next step without further purification. ES-LCMS m/z 147.1 [M+H]⁺.

### Step 8: Benzyl N-(5,6,7,8-tetrahydroisoquinolin-6-yl)carbamate

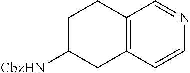 [SMILES: O=C(NC1CCc2cnccc2C1)OCc1ccccc1]

To a solution of 5,6,7,8-tetrahydroisoquinolin-6-amine (3.5 g, 23.62 mmol, 1 eq) and Na₂CO₃ (3 g, 28.30 mmol, 1.20 eq) in DCM (50 mL) and H₂O (50 mL) was added CbzCl (1.8 g, 10.55 mmol, 1.50 mL, 4.47e-1 eq) dropwise at 20° C. The mixture was stirred at 20° C. for 2 h. The reaction mixture was extracted with DCM (50 mL×3). The organic layer was dried over Na₂SO₄, filtered and concentrated under reduced pressure to yield a residue which was purified by flash silica gel chromatography (from PE/EtOAc=20/1 to 1/2, TLC: PE/EtOAc=1/1, R_{f}=0.10) to yield benzyl N-(5,6,7,8-tetrahydroisoquinolin-6-yl)carbamate (700 mg, 2.27 mmol, 9.6% yield, 91.6% purity) as a colorless gum. ¹H NMR (400 MHz, CD3OD) δ ppm 8.24 (s, 1H), 8.17 (d, J=5.2 Hz, 1H), 7.35-7.25 (m, 5H), 7.12 (d, J=5.2 Hz, 1H), 5.07 (s, 2H), 3.87 (d, J=3.6 Hz, 1H), 3.09 (dd, J=5.2, 17.2 Hz, 1H), 2.93-2.83 (m, 2H), 2.69 (dd, J=9.4, 17.2 Hz, 1H), 2.17-2.04 (m, 1H), 1.74 (dtd, J=6.0, 10.4, 12.8 Hz, 1H); ES-LCMS m/z 283.1 [M+H]⁺.

### Step 9: Benzyl N-(2-oxido-5,6,7,8-tetrahydroisoquinolin-2-ium-6-yl)carbamate

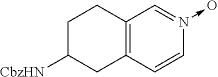 [SMILES: O=C(NC1CCc2c[n+]([O-])ccc2C1)OCc1ccccc1]

To a solution of benzyl N-(5,6,7,8-tetrahydroisoquinolin-6-yl)carbamate (700 mg, 2.27 mmol, 1 eq) in DCM (30 mL) was added m-CPBA (922.13 mg, 4.54 mmol, 85% purity, 2 eq) at 20° C. The mixture was stirred at 20° C. for 1 h. The reaction mixture was quenched with saturated aqueous Na₂S203 (20 mL), basified with saturated aqueous NaHCO₃ until pH=8 and extracted with DCM (50 mL×3). The organic layer was dried over Na₂SO₄, filtered and concentrated under reduced pressure to yield benzyl N-(2-oxido-5,6,7,8-tetrahydroisoquinolin-2-ium-6-yl)carbamate (600 mg, 2.01 mmol, 88.6% yield, N/A purity) as a white solid, which was used in the next step without further purification. ¹H NMR (400 MHz, CD3OD) δ ppm 8.13 (s, 1H), 8.05 (d, J=6.4 Hz, 1H), 7.35-7.25 (m, 6H), 5.08 (s, 2H), 3.96-3.84 (m, 1H), 3.12 (dd, J=5.4, 17.6 Hz, 1H), 2.94-2.84 (m, 2H), 2.70 (dd, J=8.8, 17.6 Hz, 1H), 2.14-2.02 (m, 1H), 1.84-1.70 (m, 1H); ES-LCMS m z 299.1 [M+H]⁺.

### Step 10: Benzyl N-(1-amino-5,6,7,8-tetrahydroisoquinolin-6-yl)carbamate

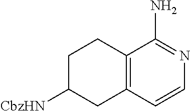 [SMILES: Nc1nccc2c1CCC(NC(=O)OCc1ccccc1)C2]

To a mixture of benzyl N-(2-oxido-5,6,7,8-tetrahydroisoquinolin-2-ium-6-yl)carbamate (580 mg, 1.94 mmol, 1 eq) and NH₃.H₂O (2.73 g, 21.81 mmol, 3 mL, 28% purity, 11.22 eq) in CHCl₃ (50 mL) was added TosCl (550 mg, 2.88 mmol, 1.48 eq). The mixture was stirred at 20° C. for 12 h. The reaction mixture was concentrated under reduced pressure. The residue was diluted with H₂O (50 mL) and extracted with EtOAc (50 mL×3). The organic layer was dried over Na₂SO₄, filtered and concentrated under reduced pressure to yield a residue which was purified by flash silica gel chromatography (from PE/EtOAc=10/1 to 0/1 and then EtOAc/MeOH=1/0 to 3/1, TLC: EtOAc, R_{f}=0.15) to yield benzyl N-(1-amino-5,6,7,8-tetrahydroisoquinolin-6-yl)carbamate (300 mg, 838.40 μmol, 43.1% yield, 83.1% purity) as a white solid. ¹H NMR (400 MHz, CD3OD) δ ppm 7.62 (d, J=5.4 Hz, 1H), 7.39-7.25 (m, 5H), 6.39 (d, J=5.6 Hz, 1H), 5.08 (s, 2H), 3.88-3.71 (m, 1H), 2.96-2.87 (m, 1H), 2.63-2.53 (m, 2H), 2.52-2.39 (m, 1H), 2.14 (dd, J=3.2, 9.6 Hz, 1H), 1.80-1.67 (m, 1H); ES-LCMS m/z 298.1 [M+H]⁺.

### Step 11: 5,6,7,8-Tetrahydroisoquinoline-1,6-diamine

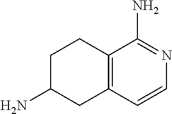 [SMILES: Nc1nccc2c1CCC(N)C2]

A mixture of benzyl N-(1-amino-5,6,7,8-tetrahydroisoquinolin-6-yl)carbamate (300 mg, 838.40 μmol, 1 eq) and Pd/C (100 mg, 10% purity) in MeOH (30 mL) was stirred under H₂ (15 Psi) at 20° C. for 12 h. The reaction mixture was filtered. The filtrate was concentrated under reduced pressure to yield 5,6,7,8-tetrahydroisoquinoline-1,6-diamine (135 mg, crude) as a white solid, which was used in the next step without further purification. ¹H NMR (400 MHz, CD3OD) δ ppm 7.62 (d, J=5.4 Hz, 1H), 6.39 (d, J=5.4 Hz, 1H), 3.08-2.98 (m, 1H), 2.85 (dd, J=4.2, 16.8 Hz, 1H), 2.61-2.52 (m, 1H), 2.50-2.42 (m, 2H), 2.14-2.05 (m, 1H), 1.60 (dtd, J=6.0, 10.4, 12.8 Hz, 1H).

### Step 12: (6R)—N₆-[2-(5-Fluoro-3-pyridyl)-8-isopropyl-pyrazolo[1,5-a][1,3,5]triazin-4-yl]-5,6,7,8-tetrahydroisoquinoline-1,6-diamine (I-63a) and (6S)—N₆-[2-(5-fluoro-3-pyridyl)-8-isopropyl-pyrazolo[1,5-a][1,3,5]triazin-4-yl]-5,6,7,8-tetrahydroisoquinoline-1,6-diamine (I-63b)

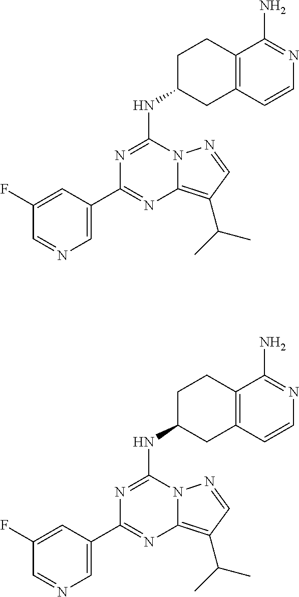 [SMILES: CC(C)c1cnn2c(N[C@@H]3CCc4c(ccnc4N)C3)nc(-c3cncc(F)c3)nc12 ; CC(C)c1cnn2c(N[C@H]3CCc4c(ccnc4N)C3)nc(-c3cncc(F)c3)nc12]

A mixture of 5,6,7,8-tetrahydroisoquinoline-1,6-diamine (135 mg, 827.11 μmol, 1 eq), 4-chloro-2-(5-fluoro-3-pyridyl)-8-isopropyl-pyrazolo[1,5-a][1,3,5]triazine (300 mg, 1.03 mmol, 1.24 eq) and DIEA (534.49 mg, 4.14 mmol, 720.34 μL, 5 eq) in i-PrOH (30 mL) was stirred at 60° C. for 2 h. The reaction mixture was concentrated under reduced pressure to yield a residue which was purified by flash silica gel chromatography (from PE/EtOAc=10/1 to 0/1 and then EtOAc/MeOH=95/5, TLC: EtOAc/MeOH=10/1, R_{f}=0.34). The desired fraction was concentrated under reduced pressure to yield the desired compound (200 mg). 140 mg of the desired compound was separated by chiral SFC (column: YMC CHIRAL Amylos-C (250 mm*30 mm, 10 um; mobile phase: [0.1% NH₃.H₂O/EtOH]; B %: 45%-45%) to yield peak 1 and peak 2. One of the peaks was concentrated under reduced pressure to yield a residue which was purified by preparative HPLC (column: Phenomenex Synergi C18 150*30 mm*4 um; mobile phase: [water (0.05% HCl)−ACN]; B %: 36%-66%, 10 min). The desired fraction was lyophilized to yield an enantiomer (47.51 mg, 89.65 μmol, 10.8% yield, 99.6% purity, 3HCl, SFC: Rₜ=2.717, ee=100%, [α]^{23.8}_{D}=−22.6 (DMSO, c=0.053 g/100 mL)) as an off-white solid. ¹H NMR (400 MHz, DMSO-d₆) δ ppm 13.75 (br s, 1H), 9.37 (s, 1H), 9.04 (d, J=8.4 Hz, 1H), 8.70 (d, J=2.8 Hz, 1H), 8.48 (dd, J=1.6, 9.6 Hz, 1H), 8.12 (s, 1H), 7.94 (br s, 2H), 7.77 (d, J=5.2 Hz, 1H), 6.70 (d, J=6.4 Hz, 1H), 4.78-4.76 (m, 1H), 3.18 (q, J=7.2 Hz, 1H), 3.14-3.01 (m, 2H), 2.68-2.57 (m, 2H), 2.19 (d, J=11.2 Hz, 1H), 2.05-1.93 (m, 1H), 1.33 (d, J=7.2 Hz, 6H); ES-LCMS m/z 419.2 [M+H]⁺. The other of the peaks was concentrated under reduced pressure to yield a residue which was purified by preparative HPLC (column: Phenomenex Synergi C18 150*30 mm*4 um; mobile phase: [water (0.05% HCl)−ACN]; B %: 32%-62%, 10 min). The desired fraction was lyophilized to yield the other enantiomer (53.69 mg, 101.71 μmol, 12.3% yield, 100.0% purity, 3HCl, SFC: Rₜ=3.575, ee=99.358%, [α]^{23.9}_{D}=+52.5 (DMSO, c=0.061 g/100 mL)) as an off-white solid. ¹H NMR (400 MHz, DMSO-d₆) δ ppm 13.63 (br s, 1H), 9.38 (t, J=1.6 Hz, 1H), 9.05 (d, J=8.4 Hz, 1H), 8.70 (d, J=2.85 Hz, 1H), 8.51-8.42 (m, 1H), 8.13 (s, 1H), 7.91 (s, 2H), 7.77 (br s, 1H), 6.71 (d, J=6.4 Hz, 1H), 4.78-4.76 (m, 1H), 3.19 (q, J=6.8 Hz, 1H), 3.15-3.01 (m, 2H), 2.69-2.60 (m, 2H), 2.20 (d, J=9.2 Hz, 1H), 2.07-1.92 (m, 1H), 1.34 (d, J=6.8 Hz, 6H); ES-LCMS m/z 419.2 [M+H]⁺.

### Example 66

### Synthesis of Compound I-64a, I-64b and I-64c

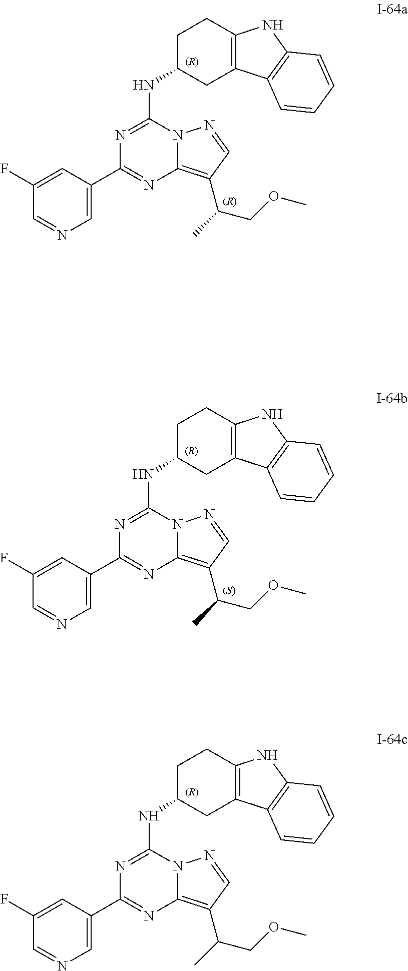

### Synthetic Scheme:

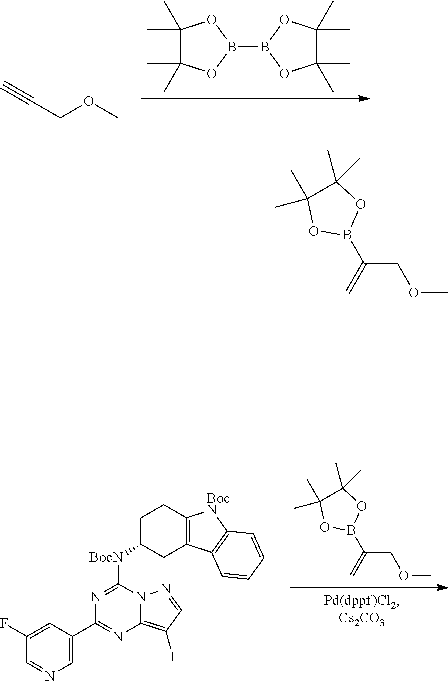

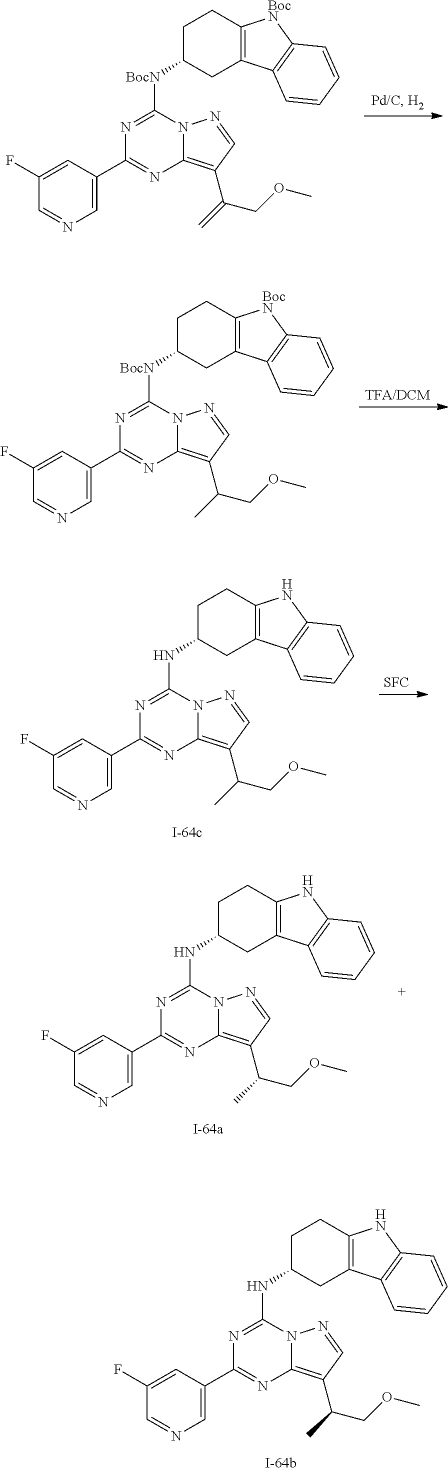

### Step 1. 2-[1-(Methoxymethyl)vinyl]-4,4,5,5-tetramethyl-1,3,2-dioxaborolane

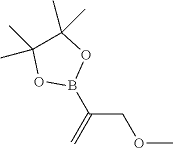

To a mixture of 3-methoxyprop-1-yne (500 mg, 7.13 mmol, 588.24 μL, 1 eq) and CuCl (70.59 mg, 713.00 μmol, 0.1 eq), 4,4,5,5-tetramethyl-2-(4,4,5,5-tetramethyl-1,3,2-dioxaborolan-2-yl)-1,3,2-dioxaborolane (1.81 g, 7.13 mmol, 1 eq), t-BuONa (102.78 mg, 1.07 mmol, 0.15 eq), t-Bu₃PBF₄ (173.10 mg, 855.60 μmol, 200.81 μL, 0.12 eq) in toluene (10 mL) was added MeOH (456.92 mg, 14.26 mmol, 577.06 μL, 2 eq) dropwise under N₂ atmosphere at 0° C. The mixture was stirred for 2 h at 20° C. The mixture was quenched by MeOH (2 mL) and filtered. The filtrate was concentrated to yield a residue which was purified by column chromatography (SiO₂, Petroleum ether/Ethyl acetate=100/1 to 10/1, R_{f}=0.75) to yield 2-[1-(methoxymethyl)vinyl]-4,4,5,5-tetramethyl-1,3,2-dioxaborolane (190 mg, 863.35 μmol, 12.1% yield, 90% purity) as colorless oil. ¹H NMR (400 MHz, CDCl₃) δ ppm 5.97-5.86 (m, 2H), 4.04 (t, J=1.8 Hz, 2H), 3.41-3.32 (m, 3H), 1.27 (s, 12H); ES-LCMS m/z 199.2 [M+H]⁺.

### Step 2: tert-Butyl (3R)-3-[tert-butoxycarbonyl-[2-(5-fluoro-3-pyridyl)-8-[1-(methoxy methyl)vinyl]pyrazolo[1,5-a][1,3,5]triazin-4-yl]amino]-1,2,3,4-tetrahydrocarbazole-9-carboxylate

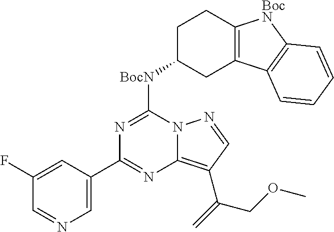 [SMILES: C=C(COC)c1cnn2c(N(C(=O)OC(C)(C)C)[C@@H]3CCc4c(c5ccccc5n4C(=O)OC(C)(C)C)C3)nc(-c3cncc(F)c3)nc12]

To a solution of tert-butyl (3R)-3-[tert-butoxycarbonyl-[2-(5-fluoro-3-pyridyl)-8-iodo-pyrazolo[1,5-a][1,3,5]triazin-4-yl]amino]-1,2,3,4-tetrahydrocarbazole-9-carboxylate (560 mg, 771.83 μmol, 1 eq) and 2-[1-(methoxymethyl)vinyl]-4,4,5,5-tetramethyl-1,3,2-dioxaborolane (169.86 mg, 771.83 μmol, 1 eq) in 1,4-dioxane (10 mL) and H₂O (2 mL) was added Cs₂CO₃ (502.95 mg, 1.54 mmol, 2 eq) and Pd(dppf)Cl₂ (56.47 mg, 77.18 μmol, 0.1 eq). The mixture was stirred at 80° C. for 2 h. The reaction mixture was concentrated under reduced pressure to yield a residue which was purified by flash silica gel chromatography (from PE/EtOAc=100/1 to 5/1, TLC: PE/EtOAc=5/1, R_{f}=0.56) to yield tert-butyl (3R)-3-[tert-butoxycarbonyl-[2-(5-fluoro-3-pyridyl)-8-[1-(methoxymethyl)vinyl]pyrazolo[1,5-a][1,3,5]triazin-4-yl]amino]-1,2,3,4-tetrahydrocarbazole-9-carboxylate (260 mg, 368.80 μmol, 47.7% yield, 95.0% purity) as a yellow solid. ¹H NMR (400 MHz, CDCl₃) δ ppm 9.49 (s, 1H), 8.60 (d, J=3.0 Hz, 1H), 8.41 (s, 1H), 8.10 (d, J=7.3 Hz, 1H), 7.34 (d, J=7.5 Hz, 2H), 7.25 (s, 1H), 6.24 (s, 1H), 5.54 (s, 1H), 4.92 (s, 2H), 4.50 (s, 2H), 3.45 (s, 3H), 3.19 (s, 4H), 2.53-2.46 (m, 1H), 2.33 (s, 1H), 1.67 (s, 9H), 1.39 (s, 9H); ES-LCMS m/z 670.3 [M+H]⁺.

### Step 3: tert-Butyl (3R)-3-[tert-butoxycarbonyl-[2-(5-fluoro-3-pyridyl)-8-(2-methoxy-1-methyl-ethyl)pyrazolo[1,5-a][1,3,5]triazin-4-yl]amino]-1,2,3,4-tetrahydrocarbazole-9-carboxylate

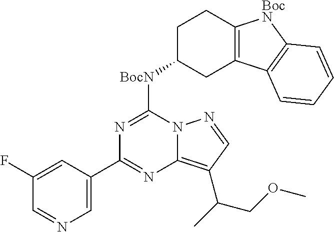 [SMILES: COCC(C)c1cnn2c(N(C(=O)OC(C)(C)C)[C@@H]3CCc4c(c5ccccc5n4C(=O)OC(C)(C)C)C3)nc(-c3cncc(F)c3)nc12]

To a solution of tert-butyl (3R)-3-[tert-butoxycarbonyl-[2-(5-fluoro-3-pyridyl)-8-[1-(methoxymethyl)vinyl]pyrazolo[1,5-a][1,3,5]triazin-4-yl]amino]-1,2,3,4-tetrahydrocarbazole-9-carboxylate (220 mg, 312.06 μmol, 1 eq) in THE (10 mL) was added Pd/C (200 mg, 10% purity) and H₂ (30 psi). The mixture was stirred at 20° C. for 1 h. The reaction mixture was filtered and the filtrate was concentrated under reduced pressure to yield a residue which was purified by preparative TLC (PE/EtOAc=0/1, R_{f}=0.35, 0.31) to yield tert-butyl (3R)-3-[tert-butoxycarbonyl-[2-(5-fluoro-3-pyridyl)-8-(2-methoxy-1-methyl-ethyl)pyrazolo[1,5-a][1,3,5]triazin-4-yl]amino]-1,2,3,4-tetrahydrocarbazole-9-carboxylate (150 mg, 196.50 μmol, 62.9% yield, 88.0% purity) as a yellow solid. ¹H NMR (400 MHz, CDCl₃) δ ppm 9.47 (s, 1H), 8.57 (d, J=2.5 Hz, 1H), 8.41 (d, J=8.3 Hz, 1H), 8.17 (s, 1H), 8.10 (d, J=8.0 Hz, 1H), 7.35 (d, J=7.8 Hz, 1H), 7.26-7.14 (m, 2H), 4.90 (s, 1H), 3.80-3.69 (m, 1H), 3.65-3.60 (m, 1H), 3.57-3.46 (m, 2H), 3.43-3.39 (m, 1H), 3.43-3.28 (m, 3H), 3.28-3.04 (m, 3H), 2.55-2.44 (m, 1H), 2.37 (s, 1H), 1.67 (s, 9H), 1.48 (d, J=6.8 Hz, 4H), 1.37 (s, 9H); ES-LCMS m/z 672.4 [M+H]⁺.

### Step 4: (3R)—N-[2-(5-Fluoro-3-pyridyl)-8-(2-methoxy-1-methyl-ethyl)pyrazolo[1,5-a][1,3,5]triazin-4-yl]-2,3,4,9-tetrahydro-1H-carbazol-3-amine

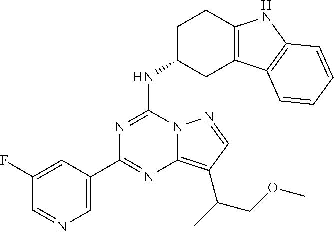 [SMILES: COCC(C)c1cnn2c(N[C@@H]3CCc4[nH]c5ccccc5c4C3)nc(-c3cncc(F)c3)nc12]

To a solution of tert-butyl (3R)-3-[tert-butoxycarbonyl-[2-(5-fluoro-3-pyridyl)-8-(2-methoxy-1-methyl-ethyl)pyrazolo[1,5-a][1,3,5]triazin-4-yl]amino]-1,2,3,4-tetrahydrocarbazole-9-carboxylate (140 mg, 183.40 μmol, 1 eq) in DCM (20 mL) was added TFA (6.18 g, 54.19 mmol, 4.01 mL, 295.46 eq). The mixture was stirred at 25° C. for 2 h. To the mixture was added water (30 mL) and extracted with DCM (30 mL×3). The combined organic phase was washed with brine (20 mL), dried with anhydrous Na₂SO₄, filtered, concentrated in vacuum to yield a residue which was purified by preparative TLC (PE/EtOAc=1/1, R_{f}=0.50) to yield (3R)—N-[2-(5-fluoro-3-pyridyl)-8-(2-methoxy-1-methyl-ethyl)pyrazolo[1,5-a][1,3,5]triazin-4-yl]-2,3,4,9-tetrahydro-1H-carbazol-3-amine (80 mg, 135.73 μmol, 74.0% yield, 80.0% purity) as a yellow solid. ¹H NMR (500 MHz, CDCl₃) δ ppm 9.52 (s, 1H), 8.57 (s, 1H), 8.47 (d, J=9.0 Hz, 1H), 7.93-7.85 (m, 2H), 7.52-7.43 (m, 1H), 7.35 (d, J=7.9 Hz, 1H), 7.19 (t, J=7.2 Hz, 1H), 7.14-7.10 (m, 1H), 6.73 (d, J=7.9 Hz, 1H), 5.00-4.90 (s, 1H), 3.81-3.69 (m, 2H), 3.65-3.56 (m, 1H), 3.40 (d, J=0.9 Hz, 4H), 3.03-2.91 (m, 3H), 2.41-2.26 (m, 2H), 1.43 (dd, J=1.7, 7.0 Hz, 3H); ES-LCMS m/z 472.2 [M+H]⁺.

### Step 5: (3R)—N-[2-(5-Fluoro-3-pyridyl)-8-[(1R)-2-methoxy-1-methyl-ethyl]pyrazolo[1,5-a][1,3,5]triazin-4-yl]-2,3,4,9-tetrahydro-1H-carbazol-3-amine (I-64a) and (3R)—N-[2-(5-fluoro-3-pyridyl)-8-[(1S)-2-methoxy-1-methyl-ethyl]pyrazolo[1,5-a][1,3,5]triazin-4-yl]-2,3,4,9-tetrahydro-1H-carbazol-3-amine (I-64b)

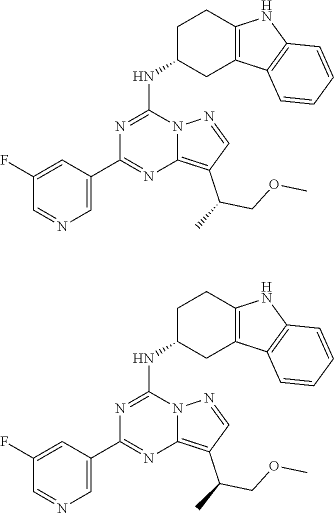 [SMILES: COC[C@H](C)c1cnn2c(N[C@@H]3CCc4[nH]c5ccccc5c4C3)nc(-c3cncc(F)c3)nc12 ; COC[C@@H](C)c1cnn2c(N[C@@H]3CCc4[nH]c5ccccc5c4C3)nc(-c3cncc(F)c3)nc12]

To a solution of (3R)—N-[2-(5-fluoro-3-pyridyl)-8-[1-(methoxymethyl) vinyl]pyrazolo[1,5-a][1,3,5]triazin-4-yl]-2,3,4,9-tetrahydro-1H-carbazol-3-amine (200 μmg, 404.67 μmol, 1 eq) in EtOAc (15 mL) was added Pd/C (200 mg, 10% purity) and H₂ (30 psi). The mixture was stirred at 20° C. for 1 h. The reaction mixture was filtered and concentrated under reduced pressure to yield a residue. To the residue was added water (30 mL) and extracted with ethyl acetate (30 mL×3). The combined organic phase was washed with brine (20 mL), dried with anhydrous Na₂SO₄, filtered and concentrated in vacuum to yield a residue which was purified by preparative HPLC (column: Phenomenex Synergi C18 150*30 mm*4 μm; mobile phase: [water (0.05% HCl)−ACN]; B %: 66%-86%, 10 min), followed by lyophilization to yield product which was separated by SFC (column: DAICEL CHIRALCEL OJ-H (250 mm*30 mm, 5 μm); mobile phase: [0.1% NH₃—H₂OEtOH]; B %: 40%-40%, min) to yield Peak 1 and Peak 2. One of these peaks was concentrated under reduced pressure to yield a residue which was dissolved in MeCN (20 mL) and H₂O (40 mL) then lyophilizzation to yield an enantiomer (19.7 mg, 41.78 μmol, 10.3% yield, 100.0% purity, Rₜ=5.188, ee=99.76%, [α]^{23.9}_{D}=+61.81 (DMSO, c=0.11 g/100 mL)) as a white solid. ¹H NMR (400 MHz, CDCl₃) δ ppm 9.52 (s, 1H), 8.56 (d, J=3.1 Hz, 1H), 8.44 (td, J=2.3, 9.5 Hz, 1H), 7.96-7.87 (m, 2H), 7.47 (d, J=7.8 Hz, 1H), 7.34 (d, J=7.8 Hz, 1H), 7.20-7.09 (m, 2H), 6.74 (d, J=8.2 Hz, 1H), 5.00-4.90 (m, 1H), 3.77-3.70 (m, 1H), 3.60 (dd, J=6.7, 9.4 Hz, 1H), 3.47-3.33 (m, 5H), 3.05-2.91 (m, 3H), 2.39-2.25 (m, 2H), 1.43 (d, J=7.0 Hz, 3H); ES-LCMS m/z 472.2 [M+H]⁺. The other of these peaks was concentrated to yield a residue which was dissolved in MeCN (20 mL) and H₂O (40 mL) then lyophilization to yield the other enantiomer (18.2 mg, 38.60 μmol, 9.5% yield, 100% purity, Rₜ=5.706 min, ee=99.23%, [α]^{23.9}_{D}=+37.89 (DMSO, c=0.095 g/100 mL)) as a white solid. ¹H NMR (400 MHz, CDCl₃) δ ppm 9.52 (s, 1H), 8.56 (d, J=2.3 Hz, 1H), 8.44 (d, J=8.6 Hz, 1H), 7.89 (s, 2H), 7.48 (d, J=7.4 Hz, 1H), 7.34 (d, J=8.2 Hz, 1H), 7.21-7.09 (m, 2H), 6.73 (d, J=8.2 Hz, 1H), 5.00-4.90 (m, 1H), 3.74 (dd, J=6.3, 9.0 Hz, 1H), 3.59 (dd, J=6.7, 9.0 Hz, 1H), 3.46-3.33 (m, 5H), 3.09-2.90 (m, 3H), 2.42-2.27 (m, 2H), 1.43 (d, J=7.0 Hz, 3H); ES-LCMS m/z 472.2 [M+H]⁺.

### Example 67

### Synthesis of Compound I-65a, I-65b and I-65c

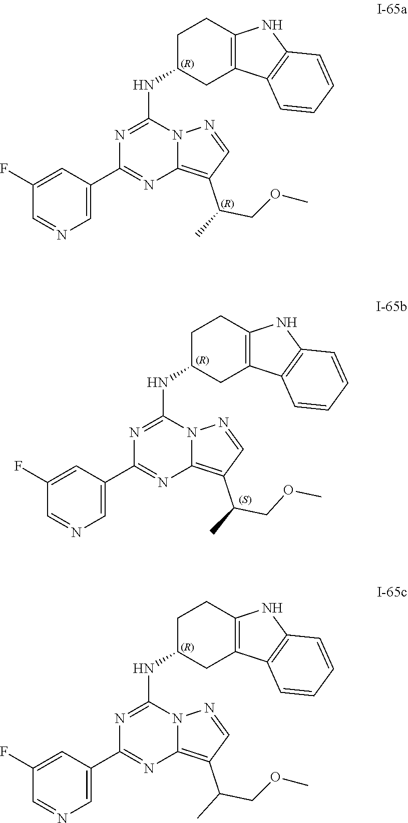

### Synthetic Scheme:

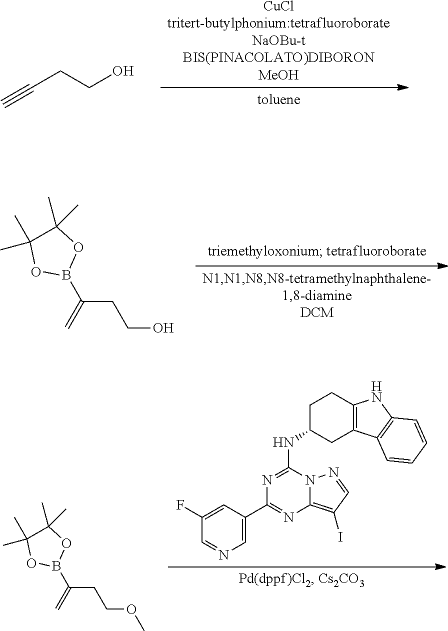

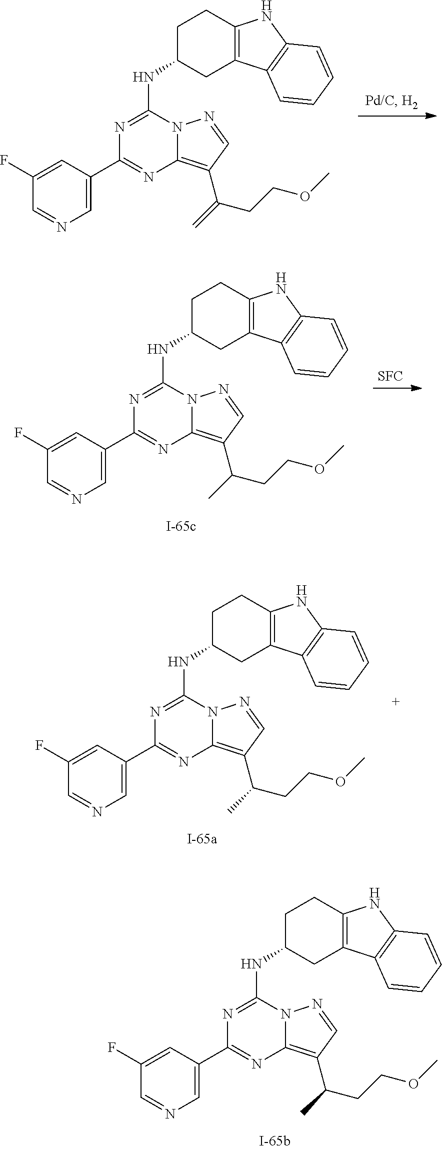

### Step 1: 3-(4,4,5,5-Tetramethyl-1,3,2-dioxaborolan-2-yl)but-3-en-1-ol

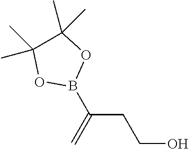

To a solution of but-3-yn-1-ol (3 g, 42.80 mmol, 3.24 mL, 1 eq) in toluene (40 mL) was added CuCl (423.74 mg, 4.28 mmol, 0.1 eq), t-BuONa (617.02 mg, 6.42 mmol, 0.15 eq) and bis(pinacolato)diboron (11.96 g, 47.08 mmol, 1.1 eq). The mixture was degassed and purged with N₂ for three times then tritert-butylphosphonium; tetrafluoroborate (1.49 g, 5.14 mmol, 0.12 eq) was added the reaction mixture. The mixture was degassed and purged with N₂ for three times. Then to the mixture was added MeOH (2.74 g, 85.60 mmol, 3.46 mL, 2 eq) dropwise at 0° C. under N₂ atmosphere. The whole mixture was stirred at 0° C. for 1 h. The reaction was quenched with MeOH (10 mL), filtered through a pad of celite and washed with DCM (10 mL×2). The filtrate was concentrated to yield a residue which was purified by flash silica gel chromatography (from PE/EtOAc=10/1 to 5/1, TLC: PE/EtOAc=1/1, R_{f}=0.50) to yield 3-(4,4,5,5-tetramethyl-1,3,2-dioxaborolan-2-yl)but-3-en-1-ol (1.6 g, 8.08 mmol, 18.9% yield) as white oil. ¹H NMR (400 MHz, CDCl₃) δ ppm 5.91 (s, 1H), 5.72 (s, 1H), 3.69 (s, 2H), 2.44 (s, 2H), 1.28 (s, 12H); ES-LCMS m z 199.1 [M+H]⁺.

### Step 2: 2-(3-Methoxy-1-methylene-propyl)-4,4,5,5-tetramethyl-1,3,2-dioxaborolane

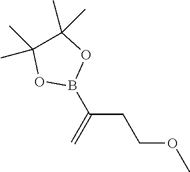

To a solution of 3-(4,4,5,5-tetramethyl-1,3,2-dioxaborolan-2-yl)but-3-en-1-ol (1.6 g, 8.08 mmol, 1 eq) in DCM (20 mL) was added trimethyloxonium; tetrafluoroborate (1.43 g, 9.69 mmol, 1.2 eq) and N1,N1,N8,N8-tetramethylnaphthalene-1,8-diamine (2.08 g, 9.69 mmol, 1.2 eq). The mixture was stirred at 20° C. for 12 h. The mixture was filtered and the filtered cake was washed with DCM (10 mL×2). The filtrate was concentrated to yield a residue which was purified by flash silica gel chromatography (from PE/EtOAc=10/1 to 3/1, TLC: PE/EtOAc=3/1, R_{f}=0.60) to yield 2-(3-methoxy-1-methylene-propyl)-4,4,5,5-tetramethyl-1,3,2-dioxaborolane (970 mg, 3.98 mmol, 49.3% yield, 87.0% purity) as yellow oil. ¹H NMR (400 MHz, CDCl₃) δ ppm 5.84 (d, J=2.7 Hz, 1H), 5.69 (s, 1H), 3.47 (t, J=7.0 Hz, 2H), 3.33 (d, J=1.2 Hz, 3H), 2.44 (t, J=6.7 Hz, 2H), 1.27 (s, 12H); ES-LCMS m/z 213.1 [M+H]⁺.

### Step 3: (3R)—N-[2-(5-Fluoro-3-pyridyl)-8-(3-methoxy-1-methylene-propyl)pyrazolo[1,5-a][1,3,5]triazin-4-yl]-2,3,4,9-tetrahydro-1H-carbazol-3-amine

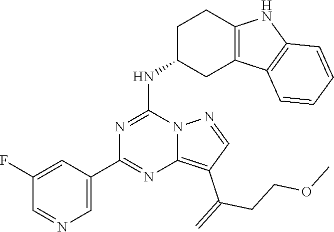 [SMILES: C=C(CCOC)c1cnn2c(N[C@@H]3CCc4[nH]c5ccccc5c4C3)nc(-c3cncc(F)c3)nc12]

To a solution of 2-(3-methoxy-1-methylene-propyl)-4,4,5,5-tetramethyl-1,3,2-dioxaborolane (350 mg, 1.44 mmol, 1.10 eq), (3R)—N-[2-(5-fluoro-3-pyridyl)-8-iodo-pyrazolo[1,5-a][1,3,5]triazin-4-yl]-2,3,4,9-tetrahydro-1H-carbazol-3-amine (800 mg, 1.31 mmol, 1 eq) and Cs₂CO₃ (853.44 mg, 2.62 mmol, 2 eq) in 1,4-dioxane (9 mL) and H₂O (3 mL) was added Pd(dppf)Cl₂ (95.83 mg, 130.97 μmol, 0.1 eq). The mixture was degassed and purged with N₂ for three times then stirred at 110° C. for 30 min under N₂ atmosphere. The reaction mixture was combined with another batch in page ES10655-104. To the mixture was added water (20 mL) and extracted with EtOAc (30 mL×3), dried with anhydrous Na₂SO₄, filtered and concentrated in vacuum to yield a residue which was purified by flash silica gel chromatography (from PE/EtOAc=100/1 to 20/1, TLC: PE/EtOAc=1/1, R_{f}=0.60) to yield (3R)—N-[2-(5-fluoro-3-pyridyl)-8-(3-methoxy-1-methylene-propyl)pyrazolo[1,5-a][1,3,5]triazin-4-yl]-2,3,4,9-tetrahydro-1H-carbazol-3-amine (430 mg, 835.03 μmol, 63.8% yield, 93.9% purity) as a yellow solid. ¹H NMR (400 MHz, CDCl₃) δ ppm 9.53 (s, 1H), 8.58 (s, 1H), 8.45 (d, J=1.5, 9.5 Hz, 1H), 8.15-8.04 (m, 1H), 7.92 (s, 1H), 7.48 (d, J=7.6 Hz, 1H), 7.35 (d, J=7.8 Hz, 1H), 7.19 (t, J=7.6 Hz, 1H), 7.16-7.10 (m, 1H), 6.78 (d, J=7.6 Hz, 1H), 6.01-5.89 (m, 1H), 5.21 (s, 1H), 4.98 (s, 1H), 3.73-3.63 (m, 2H), 3.43-3.36 (m, 4H), 3.04-2.95 (m, 5H), 2.42-2.33 (m, 2H); ES-LCMS m/z 484.4 [M+H]⁺.

### Step 4: (3R)—N-[2-(5-Fluoro-3-pyridyl)-8-[(1S)-3-methoxy-1-methyl-propyl]pyrazolo[1,5-a][1,3,5]triazin-4-yl]-2,3,4,9-tetrahydro-1H-carbazol-3-amine (I-65a) and (3R)—N-[2-(5-fluoro-3-pyridyl)-8-[(1R)-3-methoxy-1-methyl-propyl]pyrazolo[1,5-a][1,3,5]triazin-4-yl]-2,3,4,9-tetrahydro-1H-carbazol-3-amine (I-65b)

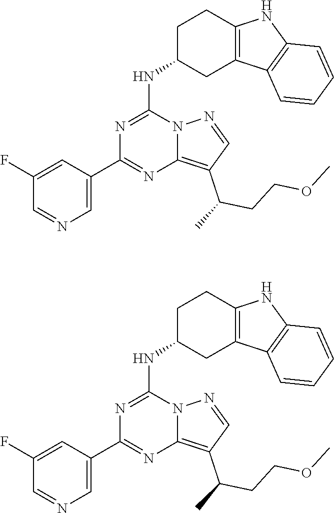 [SMILES: COCC[C@H](C)c1cnn2c(N[C@@H]3CCc4[nH]c5ccccc5c4C3)nc(-c3cncc(F)c3)nc12 ; COCC[C@@H](C)c1cnn2c(N[C@@H]3CCc4[nH]c5ccccc5c4C3)nc(-c3cncc(F)c3)nc12]

To a solution of (3R)—N-[2-(5-fluoro-3-pyridyl)-8-(3-methoxy-1-methylene-propyl)pyrazolo[1,5-a][1,3,5]triazin-4-yl]-2,3,4,9-tetrahydro-1H-carbazol-3-amine (430 μmg, 835.03 μmol, 1 eq) in EtOH (5 mL) and EtOAc (5 mL) was added Pd/C (400 mg, 10% purity) under N₂ atmosphere and the mixture was stirred under H₂ (30 psi) atmosphere. The mixture was stirred at 20° C. for 1 h. The mixture was filtered and the filtered cake was washed with EtOAc (15 mL×3) then the filtrate was concentrated to yield a residue which was purified by flash silica gel chromatography (from PE/EtOAc=100/1 to 20/1, TLC: PE/EtOAc=1/1, R_{f}=0.6) to yield the product which was separated by SFC (basic condition; column: DAICEL CHIRALPAK AS-H (250 mm*30 mm, 5 um); mobile phase: [0.1% NH₃.H₂OEtOH]; B %: 40%-40%) to yield peak 1 and peak 2. One of these peaks was concentrated and then lyophilization to yield an enantiomer (72.33 mg, 144.57 μmol, 17.3% yield, 97.1% purity, Rₜ=6.493, ee=100.0%, [α]^{19.5}_{D}=+22.64 (MeOH, c=0.106 g/100 mL)) as a white solid. ¹H NMR (400 MHz, CD3OD) δ ppm 9.39 (s, 1H), 8.52 (d, J=2.7 Hz, 1H), 8.48 (td, J=2.0, 9.7 Hz, 1H), 7.95 (s, 1H), 7.37 (d, J=7.6 Hz, 1H), 7.27 (d, J=8.1 Hz, 1H), 7.03 (t, J=7.2 Hz, 1H), 6.98-6.93 (m, 1H), 4.85-4.81 (m, 1H), 3.43-3.34 (m, 2H), 3.30-3.23 (m, 5H), 3.07-2.88 (m, 3H), 2.41-2.34 (m, 1H), 2.29-2.20 (m, 1H), 2.12-2.03 (m, 1H), 1.96 (d, J=6.7, 13.5 Hz, 1H), 1.40 (d, J=7.0 Hz, 3H); ES-LCMS m/z 486.2. The other of these peaks was concentrated under reduced pressure and then lyophilization to yield the other enantiomer (79.71 mg, 157.09 μmol, 18.8% yield, 95.7% purity, Rₜ=6.912, ee=96.96%, [α]^{19.6}_{D}=+18.00 (MeOH, c=0.100 g/100 mL)) as a white solid. ¹H NMR (400 MHz, CD3OD) δ ppm 9.42 (s, 1H), 8.52 (s, 2H), 7.98 (d, J=3.4 Hz, 1H), 7.39 (d, J=7.6 Hz, 1H), 7.28 (d, J=8.1 Hz, 1H), 7.06-7.02 (m, 1H), 6.98-6.94 (m, 1H), 4.85 (s, 1H), 3.43-3.36 (m, 2H), 3.29 (d, J=1.2 Hz, 5H), 3.07-2.91 (m, 3H), 2.39 (s, 1H), 2.36-2.35 (m, 1H), 2.12-2.06 (m, 1H), 2.02-1.96 (m, 1H), 1.43-1.40 (m, 3H); ES-LCMS m/z 486.3 [M+H]⁺.

### Example 68

### Synthesis of Compound I-66a, I-66b and I-66c

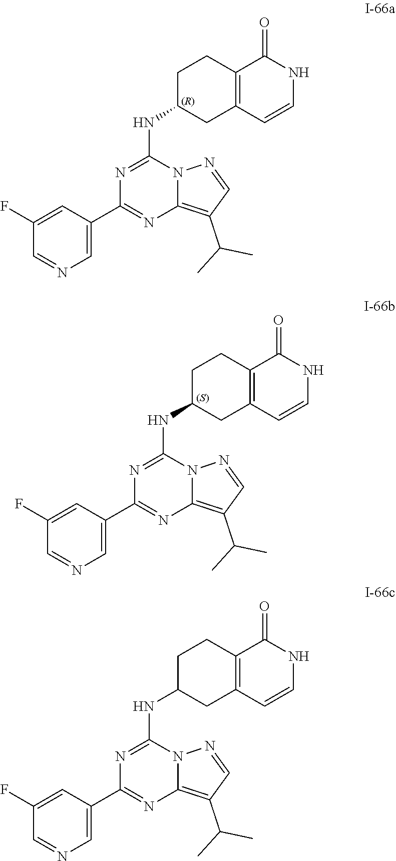

### Synthetic Scheme:

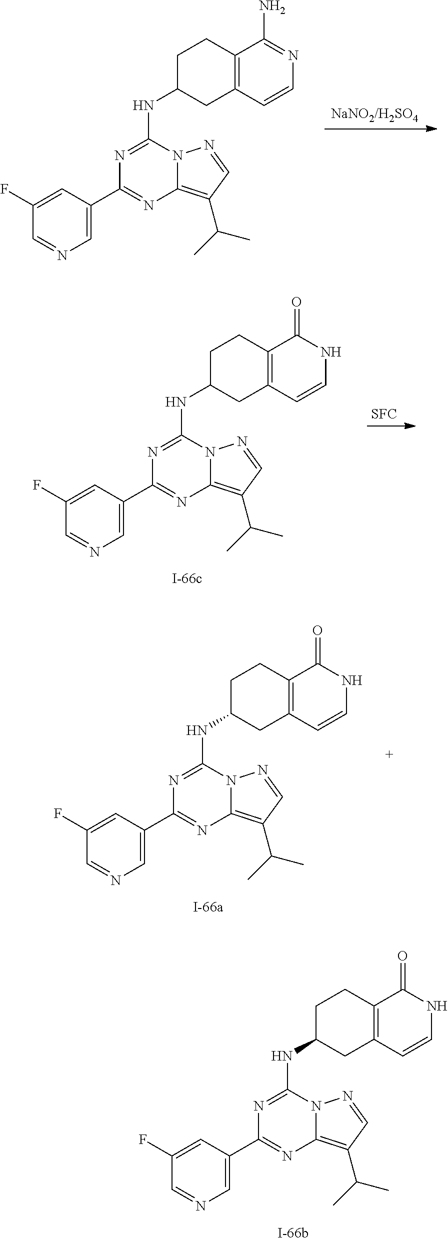

### Step 1: (6R)-6-[[2-(5-Fluoro-3-pyridyl)-8-isopropyl-pyrazolo[1,5-a][1,3,5]triazin-4-yl]amino]-5,6,7,8-tetrahydro-2H-isoquinolin-1-one (I-66a) and (6S)-6-[[2-(5-fluoro-3-pyridyl)-8-isopropyl-pyrazolo[1,5-a][1,3,5]triazin-4-yl]amino]-5,6,7,8-tetrahydro-2H-isoquinolin-1-one (I-66b)

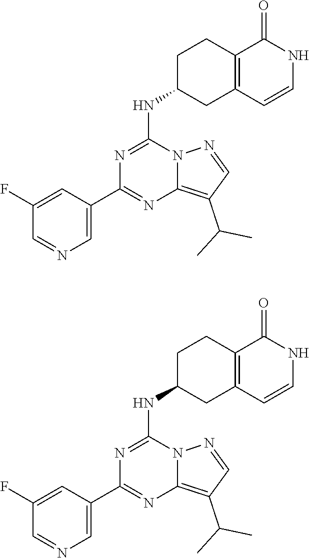 [SMILES: CC(C)c1cnn2c(N[C@@H]3CCc4c(cc[nH]c4=O)C3)nc(-c3cncc(F)c3)nc12 ; CC(C)c1cnn2c(N[C@H]3CCc4c(cc[nH]c4=O)C3)nc(-c3cncc(F)c3)nc12]

To H₂O (2 mL) was added H₂SO₄ (11.04 g, 112.56 mmol, 6.00 mL, 785.07 eq). N6-[2-(5-fluoro-3-pyridyl)-8-isopropyl-pyrazolo[1,5-a][1,3,5]triazin-4-yl]-5,6,7,8-tetrahydro isoquinoline-1,6-diamine (60 mg, 143.38 μmol, 1 eq) was added. The solution was cooled to 0° C. and NaNO₂ (9.89 mg, 143.38 μmol, 1 eq) was added. The mixture was stirred at 0° C. for 0.5 h and then at 75° C. for 12 h. The reaction mixture was diluted with H₂O (20 mL), basified with aqueous NaOH (15%) until pH=8 and extracted with EtOAc (30 mL×3). The organic layer was dried over Na₂SO₄, filtered and concentrated under reduced pressure. The residue was separated by chiral SFC (column: DAICEL CHIRALPAK AD-H (250 mm*30 mm, 5 um); mobile phase: [0.1% NH₃.H₂O/IPA]; B %: 35%-35%) to yield peak 1 and peak 2. One of these peaks was concentrated to yield a residue which was purified by preparative HPLC (column: Phenomenex Synergi C18 150*30 mm*4 um; mobile phase: [water (0.05% HCl)−ACN]; B %: 49%-69%, 10 min). The desired fraction was concentrated under reduced pressure. The residue was diluted with water (20 mL), basified with saturated aqueous Na₂CO₃ until pH=9 and extracted with EtOAc (50 mL×5). The organic layer was dried over Na₂SO₄, filtered and concentrated under reduced pressure. The residue was lyophilized to yield an enantiomer (22.78 mg, 35.14% yield, 100% purity, SFC: Rₜ=5.546, ee=100.000%, [α]^{20.8}_{D}=−27.6 (DMSO, c=0.058 g/100 mL)) as a white solid. ¹H NMR (400 MHz, DMSO-d₆) δ ppm 11.29 (br s, 1H), 9.36 (s, 1H), 8.93 (d, J=8.4 Hz, 1H), 8.68 (d, J=2.8 Hz, 1H), 8.43 (d, J=9.6 Hz, 1H), 8.11 (s, 1H), 7.12 (d, J=6.0 Hz, 1H), 5.93 (d, J=6.8 Hz, 1H), 4.70-4.60 (m, 1H), 3.30-3.26 (m, 1H), 3.20-3.10 (m, 1H), 2.86 (d, J=7.2 Hz, 2H), 2.70-2.60 (m, 1H), 2.10-2.00 (m, 1H), 1.93-1.80 (m, 1H), 1.33 (d, J=7.2 Hz, 6H); ES-LCMS m z 420.2 [M+H]⁺. The other of these peaks was concentrated under reduced pressure to yield a residue which was purified by preparative HPLC (column: Phenomenex Synergi C18 150*30 mm*4 um; mobile phase: [water (0.05% HCl)−ACN]; B %: 49%-69%, 10 min). The desired fraction was concentrated under reduced pressure. The residue was diluted with water (20 mL), basified with saturated aqueous Na₂CO₃ until pH=9 and extracted with EtOAc (50 mL×5). The organic layer was dried over Na₂SO₄, filtered and concentrated under reduced pressure. The residue was lyophilized to yield the other enantiomer (21.73 mg, SFC: Rₜ=5.863, ee=100.000%, [α]21.0=+21.9 (DMSO, c=0.064 g/100 mL)) as a white solid. ¹H NMR (400 MHz, DMSO-d₆) δ ppm 11.29 (br s, 1H), 9.36 (s, 1H), 8.93 (d, J=8.4 Hz, 1H), 8.68 (d, J=2.8 Hz, 1H), 8.43 (d, J=8.8 Hz, 1H), 8.11 (s, 1H), 7.12 (s, 1H), 5.94 (d, J=6.8 Hz, 1H), 4.70-4.60 (m, 1H), 3.30-3.26 (m, 1H), 3.20-3.10 (m, 1H), 2.86 (d, J=7.2 Hz, 2H), 2.70-2.60 (m, 1H), 2.12-2.04 (m, 1H), 1.94-1.81 (m, 1H), 1.34 (d, J=7.2 Hz, 6H); ES-LCMS m/z 420.2 [M+H]⁺.

### Example 69

### Synthesis of Compound I-67

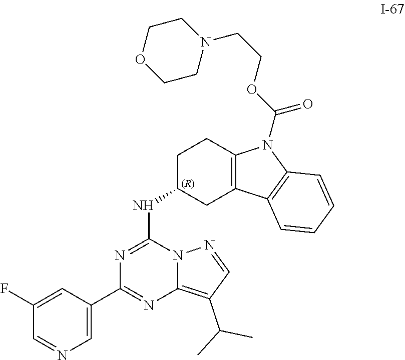

### Synthetic Scheme:

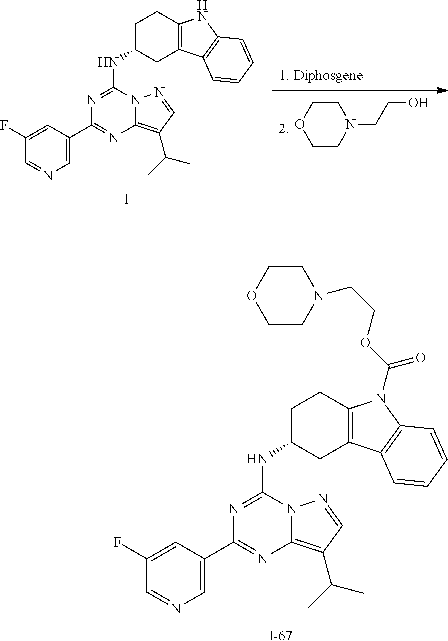

### Step 1. 2-Morpholinoethyl (3R)-3-[[2-(5-fluoro-3-pyridyl)-8-isopropyl-pyrazolo[1,5-a][1,3,5]triazin-4-yl]amino]-1,2,3,4-tetrahydrocarbazole-9-carboxylate

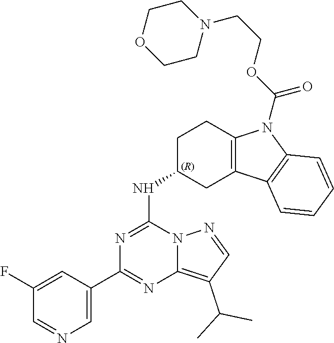

To a solution of (3R)—N-[2-(5-fluoro-3-pyridyl)-8-isopropyl-pyrazolo[1,5-a][1,3,5]triazin-4-yl]-2,3,4,9-tetrahydro-1H-carbazol-3-amine (784.00 mg, 1.78 mmol, 1 eq) in THE (3 mL) was added NaH (106.54 mg, 2.66 mmol, 60% purity, 1.5 eq). The mixture was stirred at 0° C. for 0.5 h. Then trichloromethyl carbonochloridate (1.05 g, 5.33 mmol, 642.62 μL, 3 eq) was added. The mixture was stirred at 25° C. for 12 h. To the mixture was added 2-morpholinoethanol (465.86 mg, 3.55 mmol, 435.39 μL, 2 eq). The mixture was stirred at 60° C. for 12 h. The reaction mixture was diluted with H₂O (30 mL) and extracted with EtOAc (30 mL×3). The combined organic layers were washed with brine (30 mL), dried over Na₂SO₄, filtered and concentrated under reduced pressure to give a residue which was purified by flash silica gel chromatography (from PE/EtOAc=100/1 to 0/1). The desired fraction was concentrated to yield a residue which was purified by preparative HPLC (column: Agela DuraShell C18 250*25 mm*10 um; mobile phase: [water (0.04% NH₃.H₂O+10 mM NH₄HCO₃)−ACN]; B %: 72%-100%, 10 min). The desired fraction was lyophilized to yield 2-morpholinoethyl (3R)-3-[[2-(5-fluoro-3-pyridyl)-8-isopropyl-pyrazolo[1,5-a][1,3,5]triazin-4-yl]amino]-1,2,3,4-tetrahydro carbazole-9-carboxylate (29.21 mg, 48.06 μmol, 38.2% yield, 98.5% purity, [α]^{19.6}_{D}=−10.0 (MeOH, c=0.020 g/100 mL), [α]^{20.2}_{D}=−9.1 (DMSO, c=0.022 g/100 mL)) as a white solid. ¹H NMR (500 MHz, CD3OD) δ ppm 9.43 (s, 1H), 8.63-8.47 (m, 2H), 8.22 (d, J=8.0 Hz, 1H), 8.01 (s, 1H), 7.46 (d, J=7.2 Hz, 1H), 7.33-7.20 (m, 2H), 4.90-4.88 (m, 1H), 4.66-4.52 (m, 2H), 3.72 (t, J=4.3 Hz, 4H), 3.30 (d, J=6.7 Hz, 4H), 3.01-2.82 (m, 3H), 2.63 (s, 4H), 2.42 (s, 1H), 2.27 (s, 1H), 1.44 (d, J=7.0 Hz, 6H); ES-LCMS m/z 599.4 [M+H]⁺.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

1. A compound of formula I: 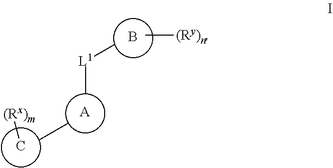
or a pharmaceutically acceptable salt thereof, wherein:
Ring A is selected from: 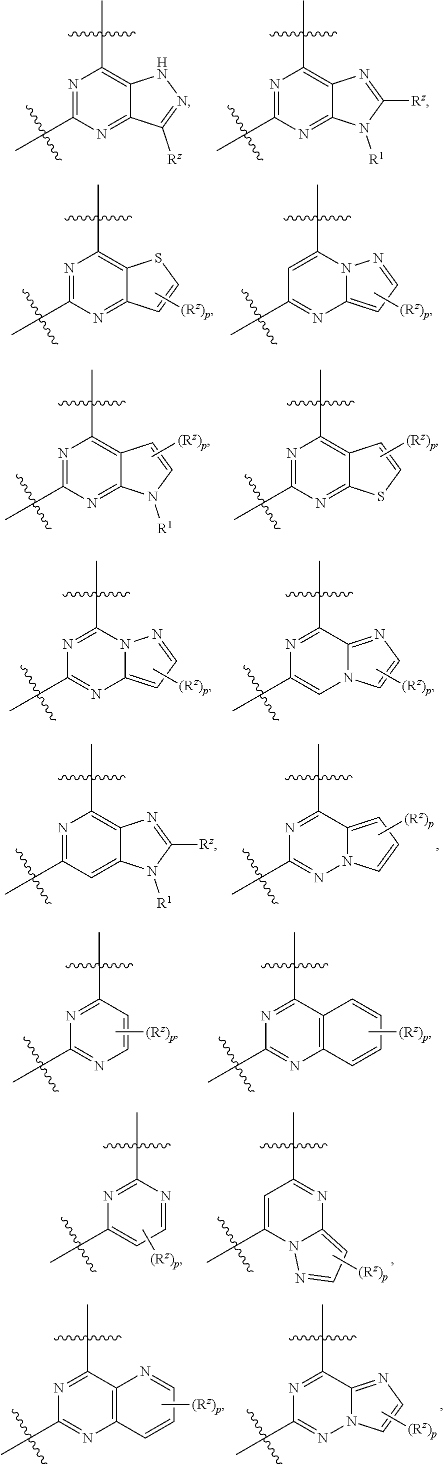 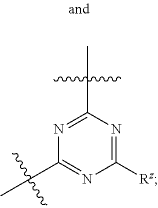
each p is independently 0, 1, or 2, as valency will allow;
each R¹ is independently selected from R, —C(O)R, —C(O)OR, —SO₂R, —C(O)N(R)₂, or —SO₂RN(R)₂;
each R is independently hydrogen, or an optionally substituted group selected from C₁₋₆ aliphatic, a 3-8 membered saturated or partially unsaturated monocyclic carbocyclic ring, phenyl, an 8-10 membered bicyclic aromatic carbocyclic ring; a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur; or two R on the same nitrogen are taken together with their intervening atoms to form a 4-7 membered saturated, partially unsaturated, or aromatic ring having 1-2 heteroatoms in addition to the nitrogen independently selected from oxygen, nitrogen, or sulfur;
each or R^{x}, R^{y}, and R^{z} is independently selected from R, halogen, cyano, nitro, —OR, —SR, —N(R)₂, —N(R)C(O)R, —C(O)N(R)₂, —C(O)N(R)OR, —N(R)C(O)N(R)₂, —N(R)C(O)OR, —OC(O)N(R)₂, —N(R)SO₂R, —SO₂RN(R)₂, —C(O)R, —C(O)OR, —OC(O)R, —C(O)OR, —S(O)R, or —SO₂R, or:
two R^{z} on adjacent atoms are taken together with their intervening atoms to form a 3-7 membered saturated, partially unsaturated, or aromatic ring having 0-2 heteroatoms independently selected from nitrogen, oxygen, or sulfur;
two R^{x} on the same carbon are taken together to form ═O or ═S; or:
two R^{y} on the same carbon are taken together to form ═O or ═S;
each of m and n is independently 1, 2, 3, 4, or 5;
Ring B is absent or a 4-8 membered saturated or partially unsaturated carbocyclic ring; phenyl, a 7-10 membered bicyclic partially unsaturated or aromatic carbocyclic ring, a 5-6 membered monocyclic heteroaromatic ring having 1-4 heteroatoms independently selected from nitrogen, oxygen, or sulfur, an 8-10 membered bicyclic partially unsaturated or heteroaryl ring having 1-4 heteroatoms independently selected from nitrogen, oxygen, or sulfur, or a 12-15 membered partially unsaturated or aromatic tricyclic ring having 1-4 heteroatoms independently selected from nitrogen, oxygen, or sulfur;
Ring C is phenyl, a 5-6 membered saturated, partially unsaturated or heteroaryl ring having 1-4 heteroatoms independently selected from nitrogen, oxygen, or sulfur, or an 8-10 membered bicyclic partially unsaturated or heteroaryl ring having 1-4 heteroatoms independently selected from nitrogen, oxygen, or sulfur;
L¹ is a covalent bond or an optionally substituted C₁₋₆ membered straight or branched bivalent hydrocarbon chain wherein a methylene unit of L¹ is optionally replaced with -Cy-, —O—, —S—, —NR—, —C(O)—, —C(O)O—, —OC(O)—, —C(O)N(R)—, —N(R)C(O)—, —SO₂—, —N(R)SO₂—, or —SO₂N(R)—S; and
-Cy- is a 3-8 membered bivalent saturated, partially unsaturated, or aromatic monocyclic ring having 0-2 heteroatoms independently selected from nitrogen, oxygen, or sulfur, or an 8-10 membered bivalent saturated, partially unsaturated, or aromatic bicyclic ring having 0-3 heteroatoms independently selected from nitrogen, oxygen, or sulfur;
with the proviso that when Ring A is 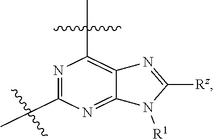
Ring B is not 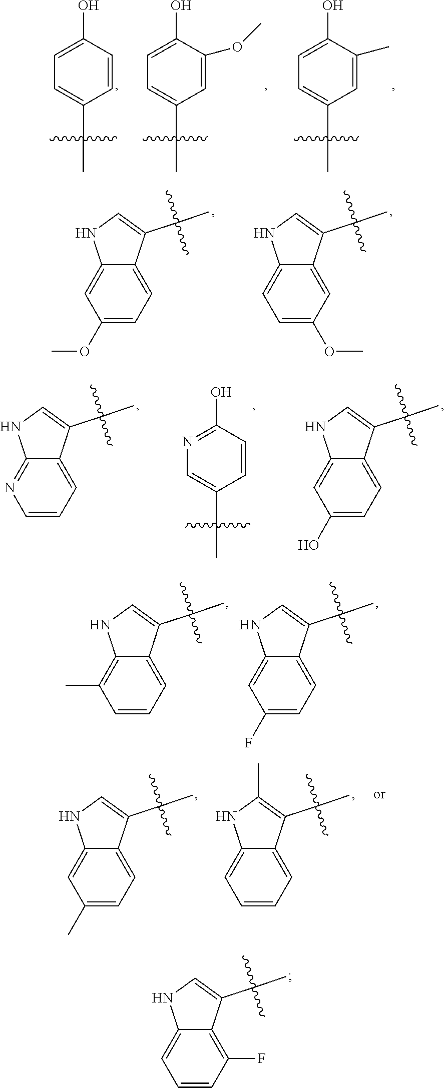
and/or Ring C is not 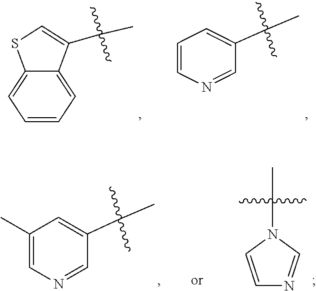
and/or R¹ is not

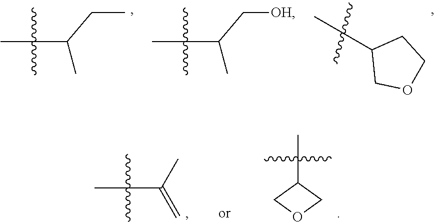

2. The compound according to claim 1 wherein the compound is selected from any of formulae I-a, I-b, I-c, I-d, I-e, I-f, I-g, I-h, I-i, I-j, I-k, I-l, I-m, I-n, I-o, I-p, I-q, I-r, I-s, I-t and I-u: 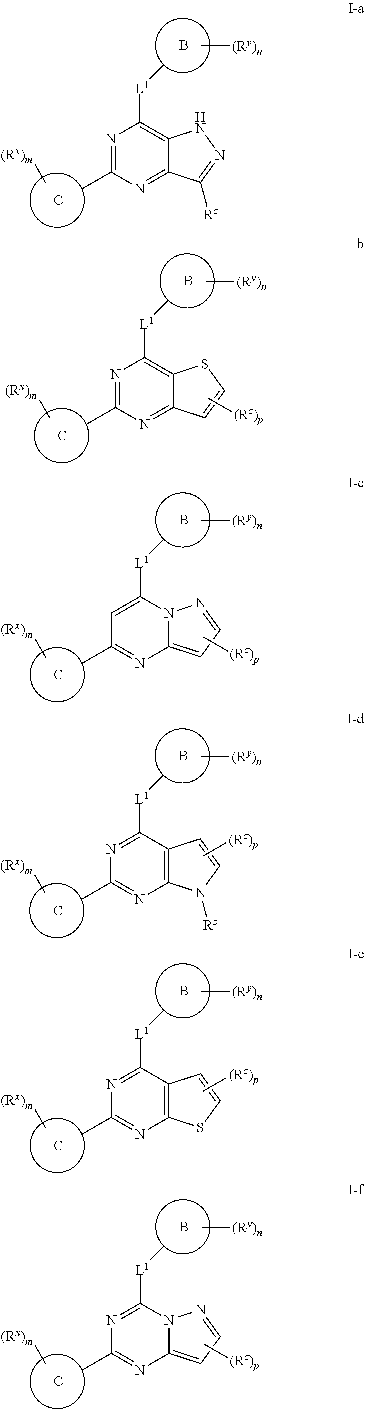 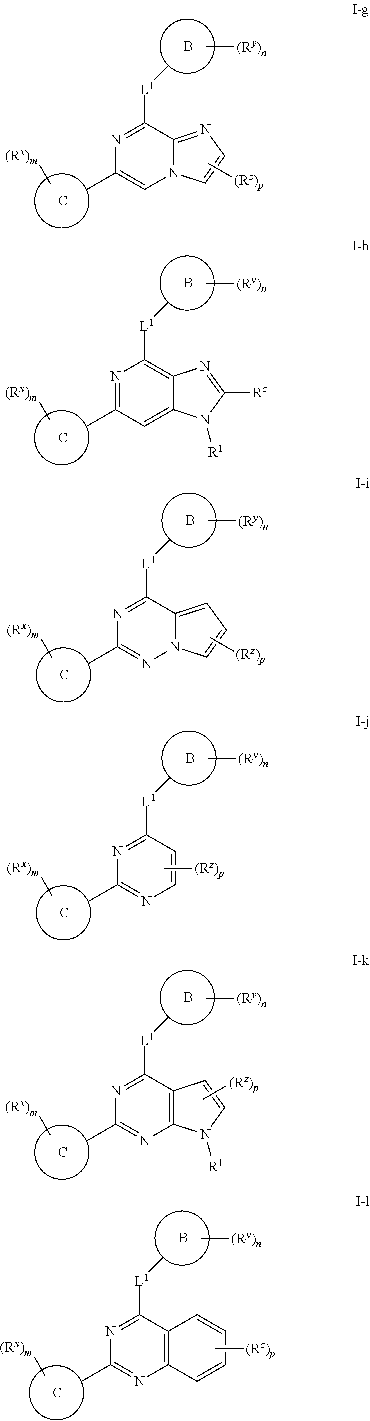 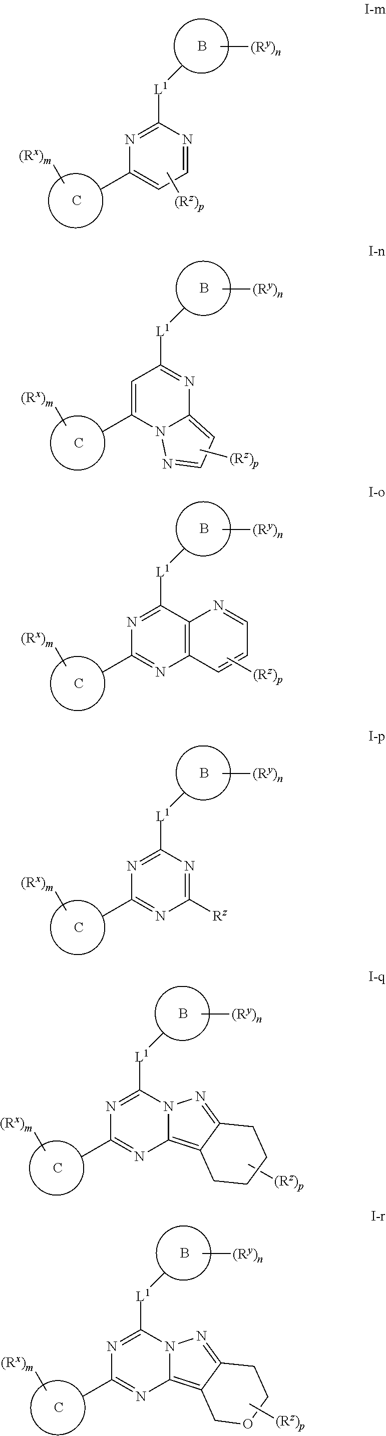 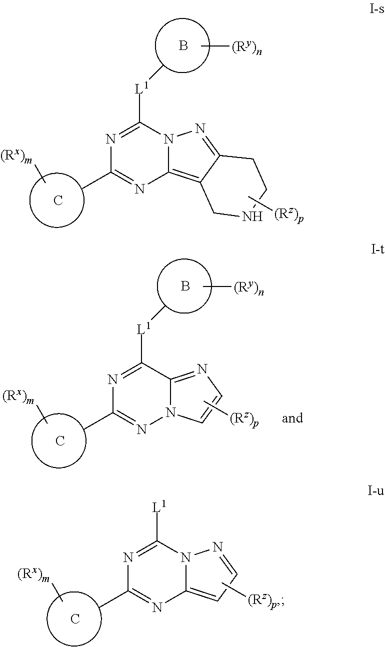
or a pharmaceutically acceptable salt thereof.

3. The compound according to claim 1 wherein the compound is selected from any of formulae II-a, II-b, II-c, II-d, II-e, II-f, II-g, II-h, II-i, II-j, II-k, II-l, II-m, II-n, II-o, II-p, II-q, II-r, II-s, II-t and II-u: 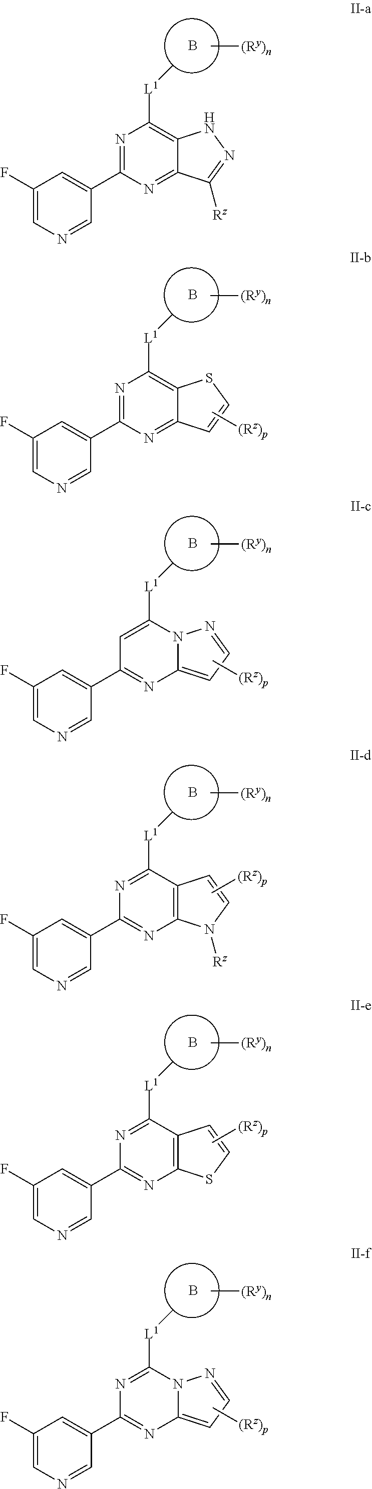 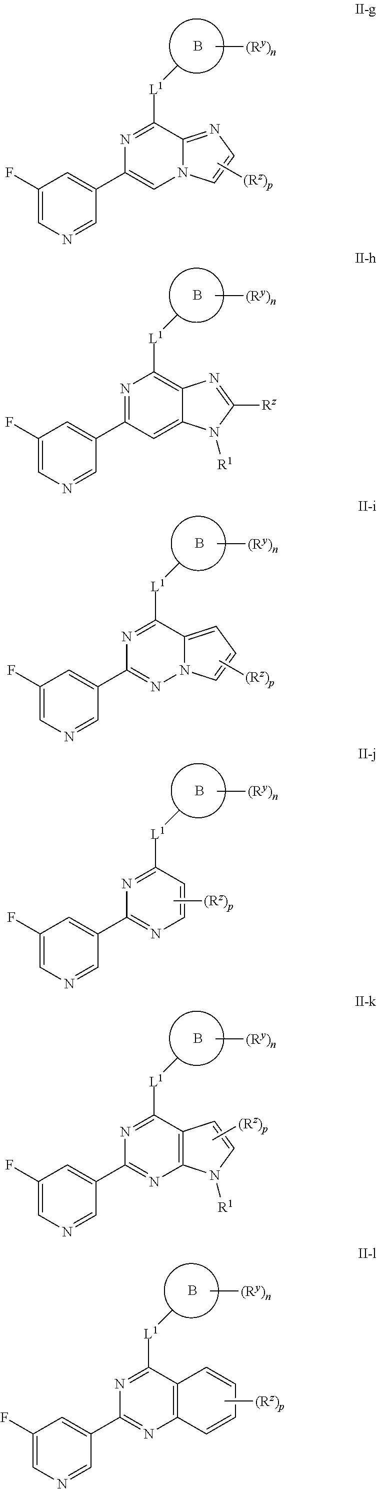 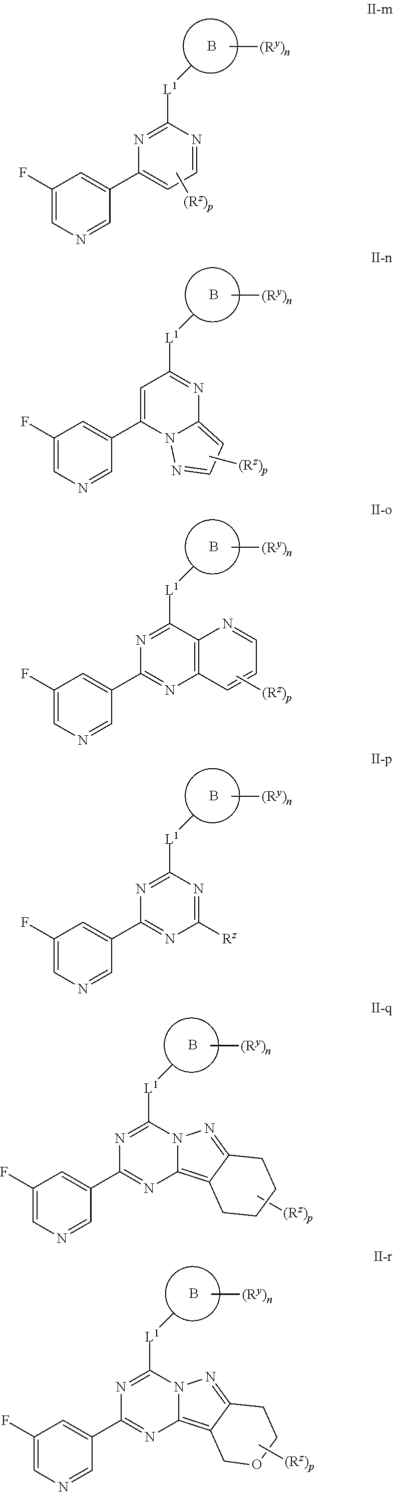 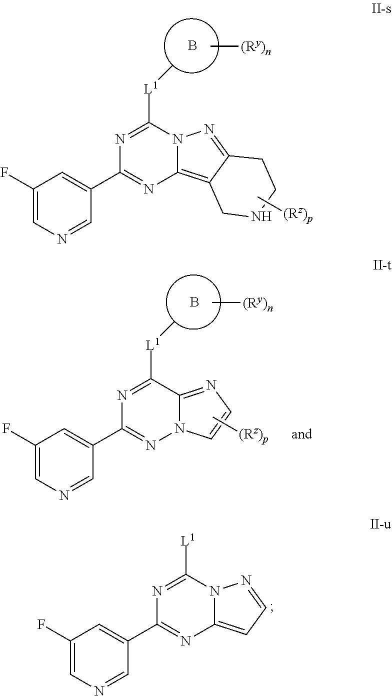
or a pharmaceutically acceptable salt thereof.

4. The compound according to claim 1 wherein the compound is selected from any of formulae III-a, III-b, III-c, III-d, III-e, III-f, III-g, III-h, III-i, III-j, III-k, III-l, III-m, III-n, III-o, III-p, III-q, III-r, III-s, III-t, III-u, III-v, III-w, III-x, III-y, III-z, III-aa, III-bb, III-cc, III-dd, III-ee, III-ff, III-gg, III-hh, III-ii, III-jj and III-kk: 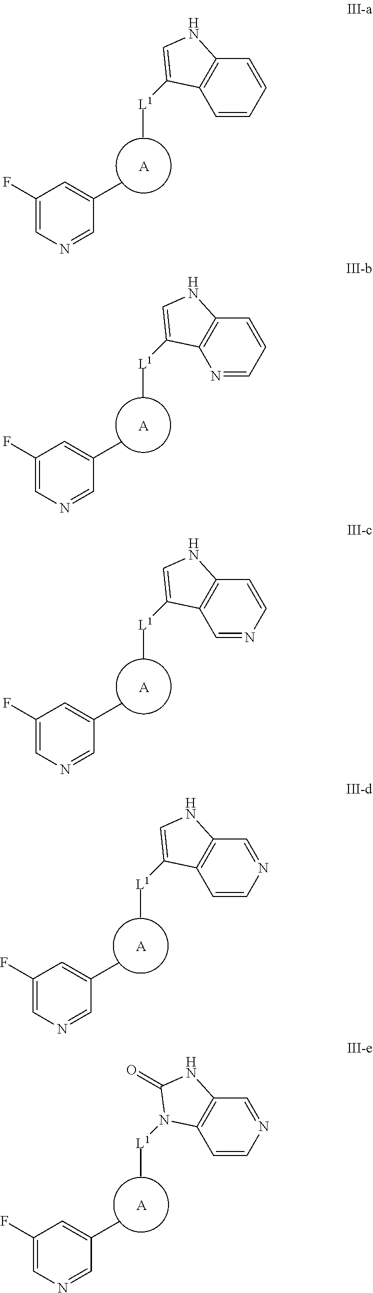 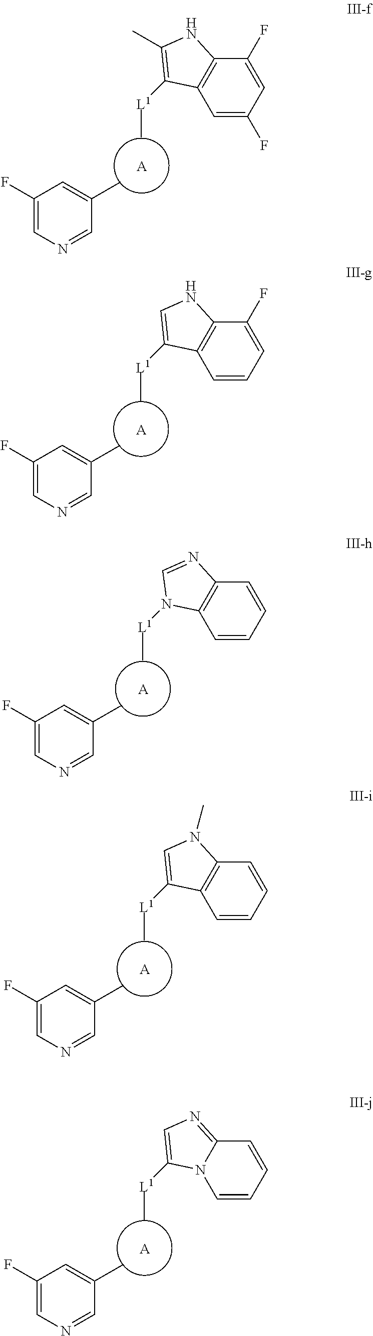 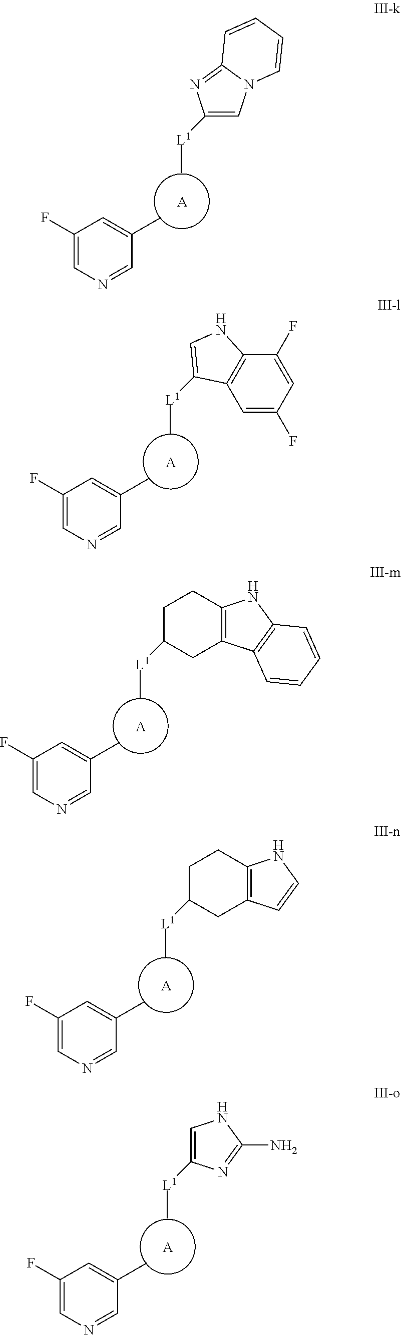 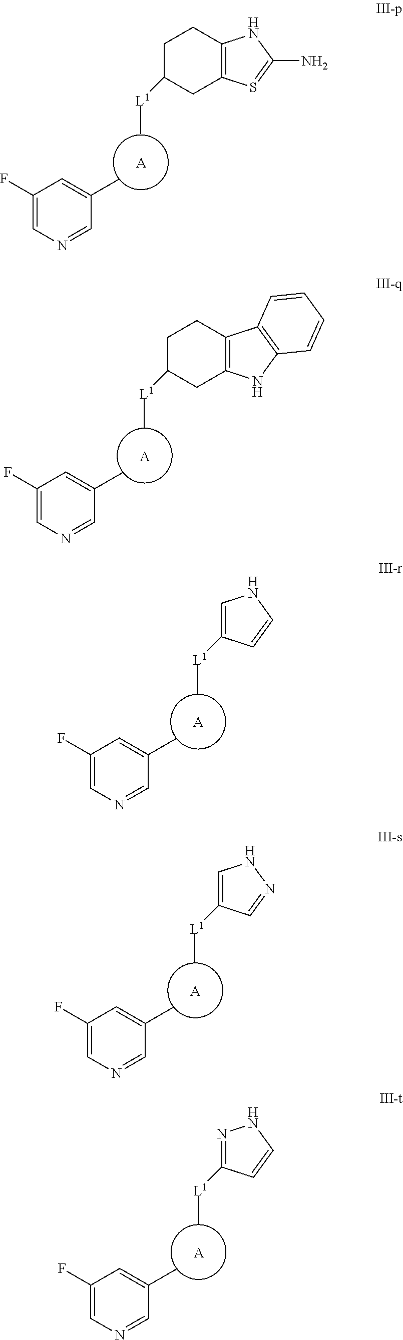 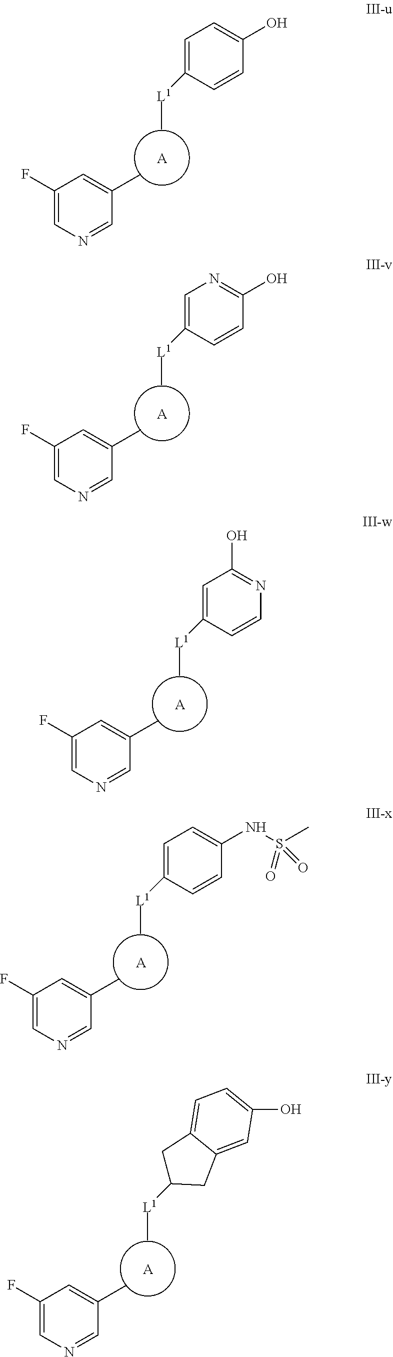 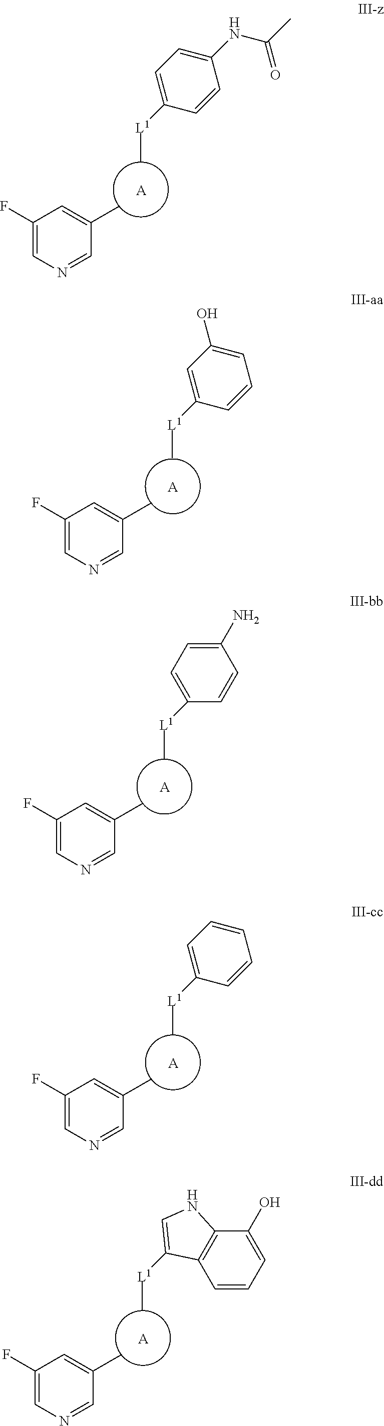 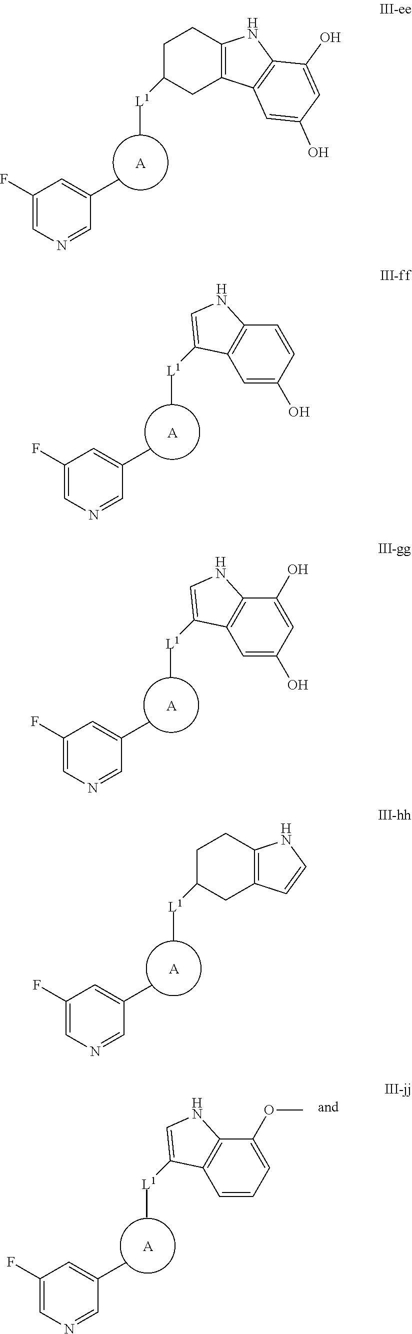 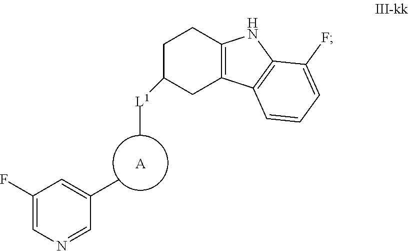
or a pharmaceutically acceptable salt thereof.

5. The compound according to claim 1 wherein the compound is selected from any of formulae IV-a, IV-b, IV-c, IV-d, IV-e, IV-f, IV-g, IV-h, IV-i, IV-j, IV-k, IV-l, IV-m, IV-n, IV-o, IV-p and IV-q: 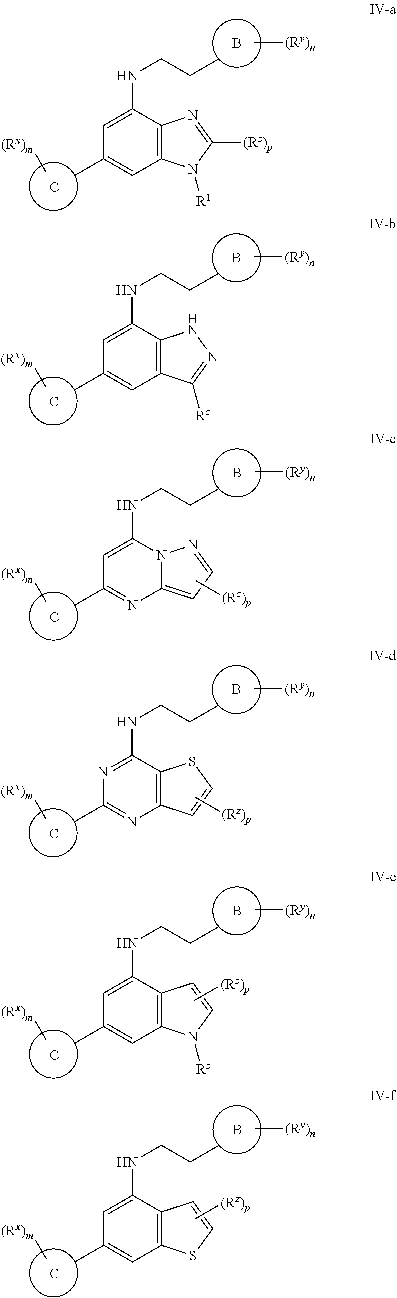 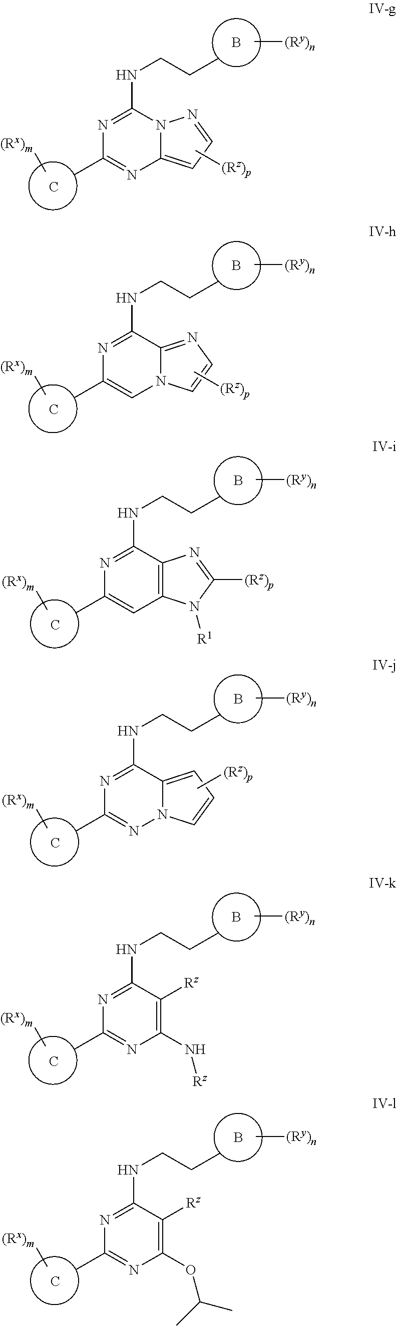 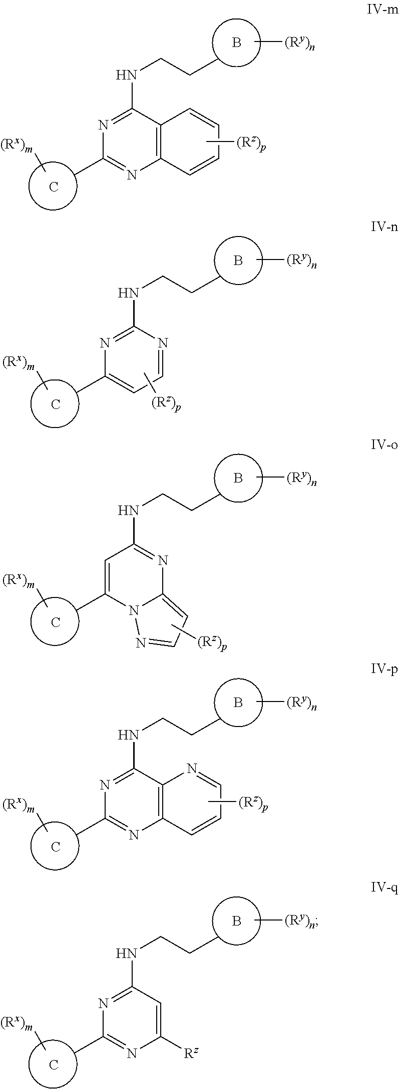
or a pharmaceutically acceptable salt thereof

6. The compound according to claim 1 wherein the compound is selected from any of formulae V-a, V-b, V-c, V-d, V-e, V-f, V-g, V-h, V-i, V-j, V-k and V-l: 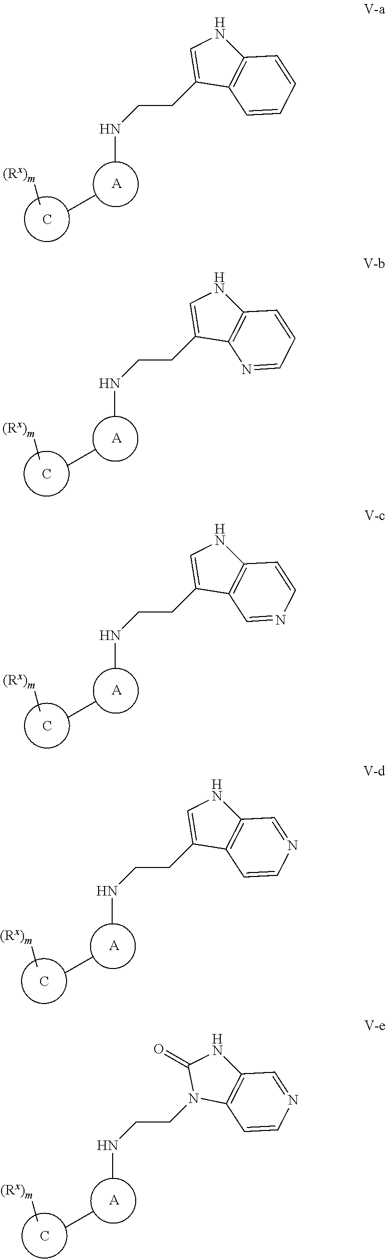 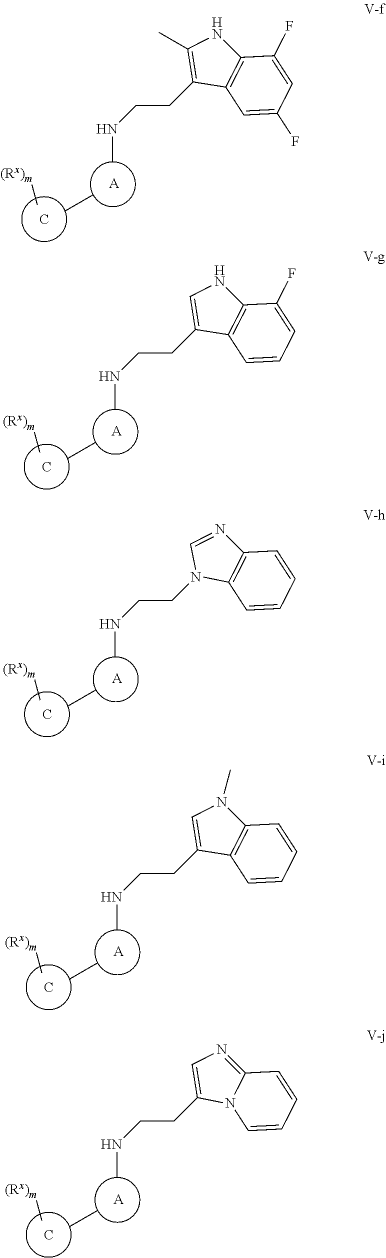 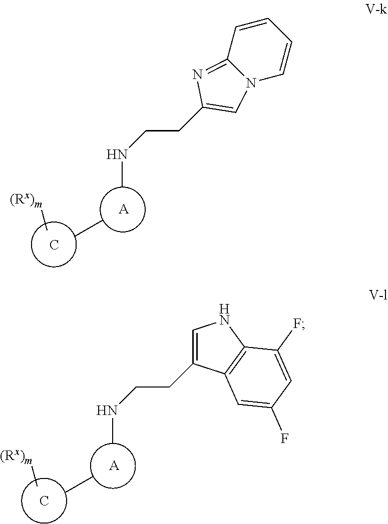
or a pharmaceutically acceptable salt thereof.

7. The compound according to claim 1 wherein the compound is selected from any of formulae VI-a, VI-b, VI-c, VI-d, VI-e, VI-f, VI-g, VI-h, VI-i, VI-j and VI-k: 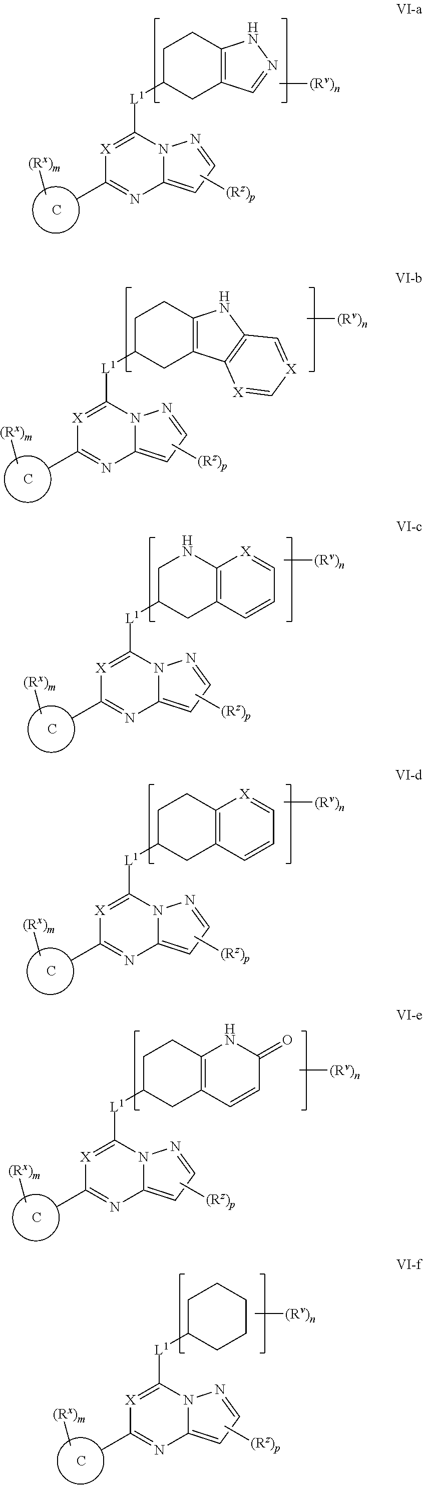 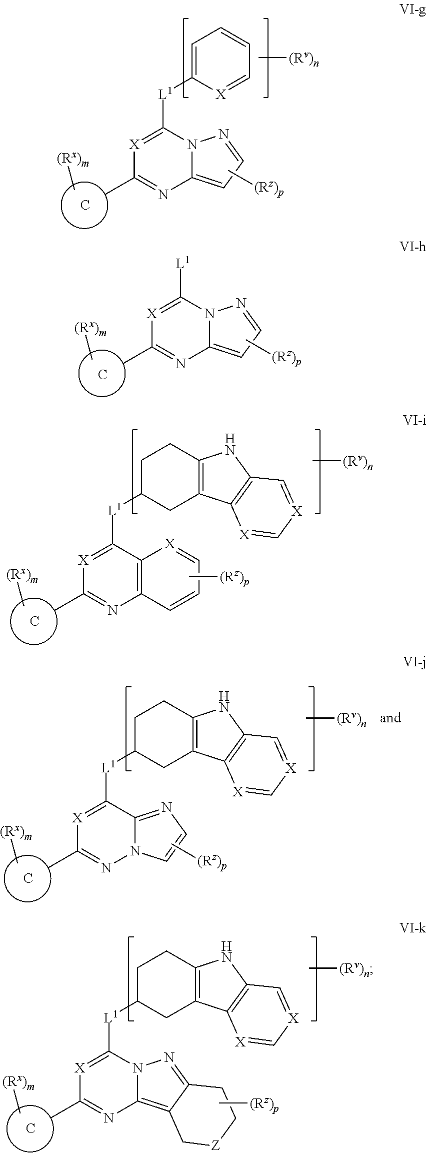
or a pharmaceutically acceptable salt thereof, wherein X is N or CH and Z is CH₂, NH or O.

8. The compound according to claim 1 wherein the compound is selected from any of formulae VII-a, VII-b, VII-c, VII-d, VII-e, VITI-f, VII-g, VITI-h, VII-i and VII-j: 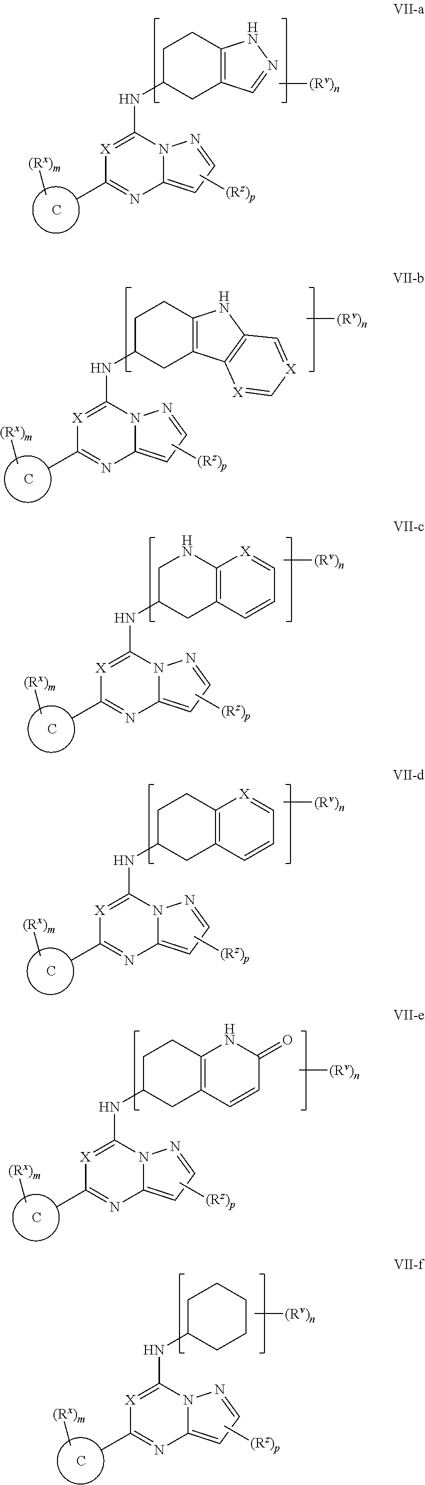 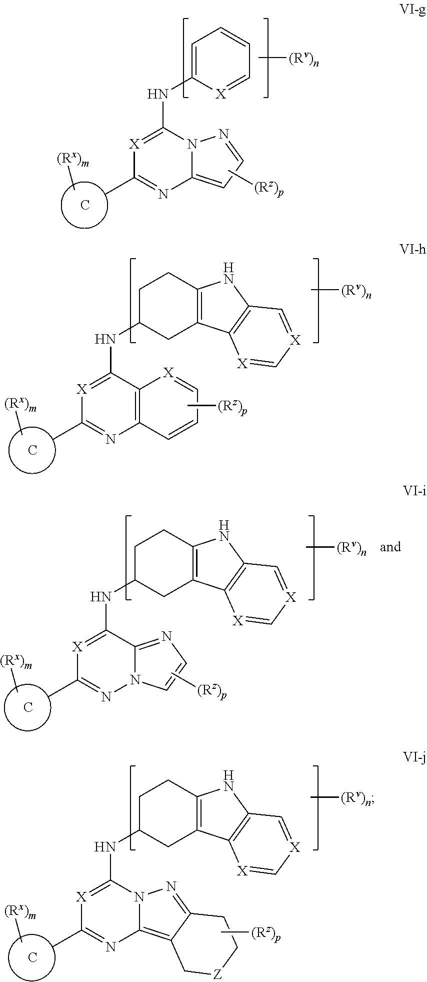
or a pharmaceutically acceptable salt thereof wherein X is N or CH and Z is CH₂, NH or O.

9. The compound according to claim 1 wherein the compound is selected from any of formulae VIII-a, VIII-b, VII-c, VIII-d, VIII-e, VIII-f, VII-g, VIII-h, VIII-i, VIII-j, VIII-k, VIII-l, VIII-m, VIII-n, VIII-o, VIII-p, VIII-q and VIII-r: 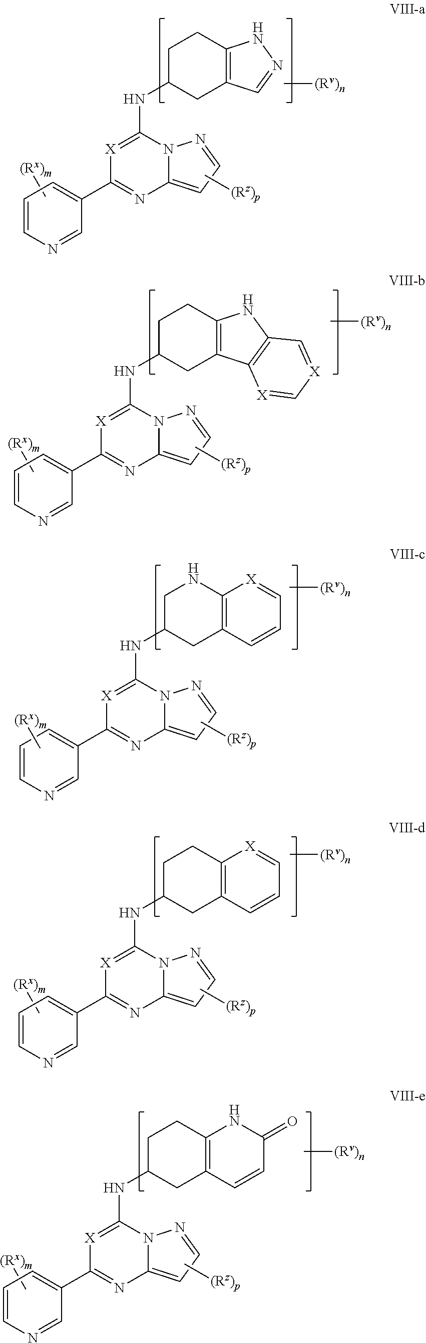 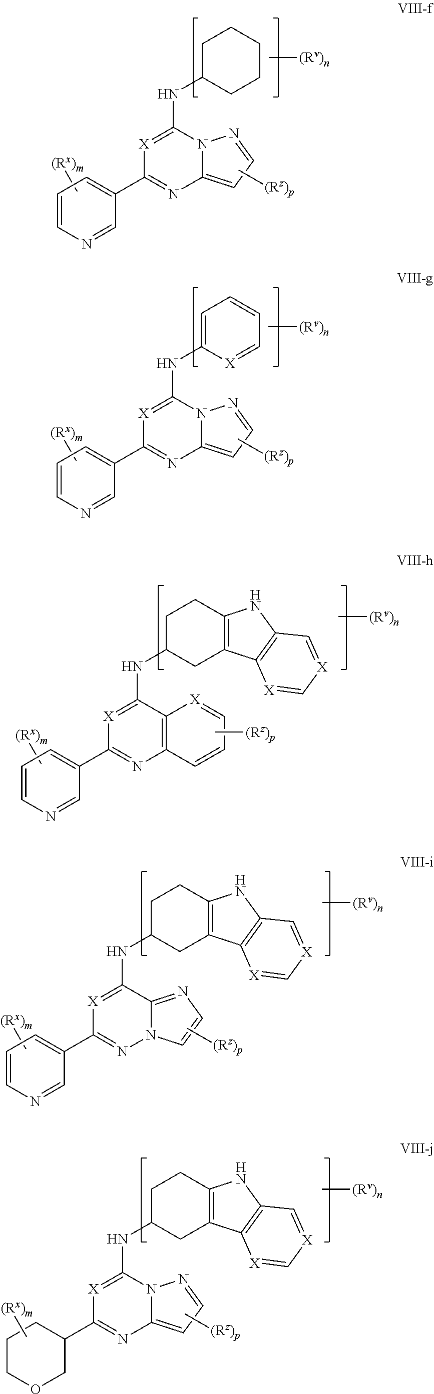 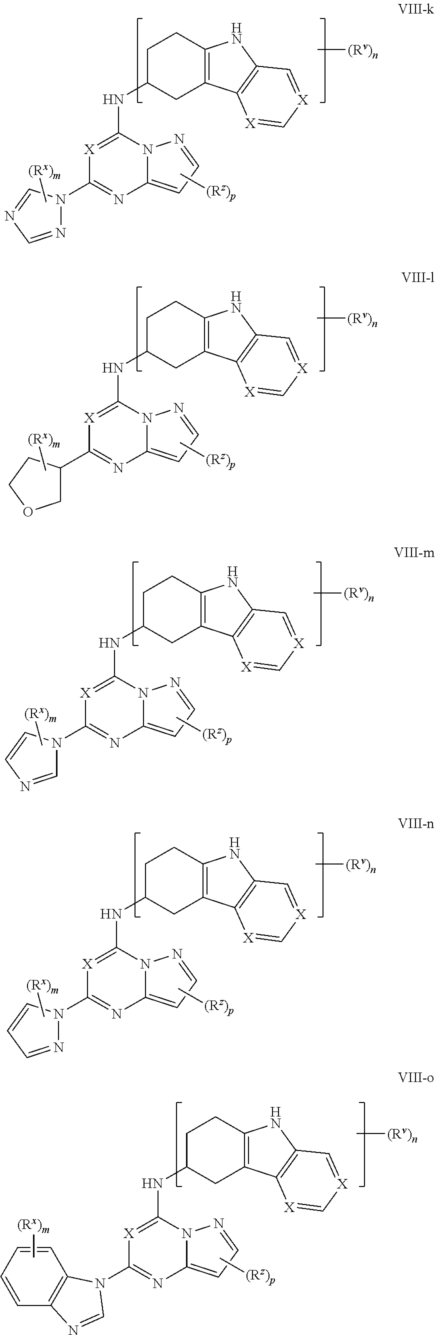 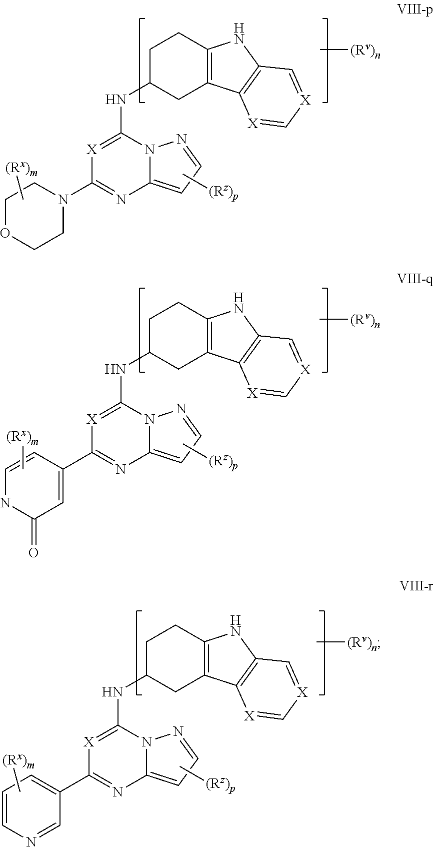
or a pharmaceutically acceptable salt thereof wherein X is N or CH and Z is CH₂, NH or O.

10. The compound according to claim 1 wherein the compound is selected from any of formulae TX-a, TX-b, TX-c, TX-d, TX-e, TX-f, IX-g, TX-h, TX-i, TX-j, TX-k, TX-l, TX-m, TX-n, IX-o, TX-p, TX-q, TX-r, TX-s and TX-t: 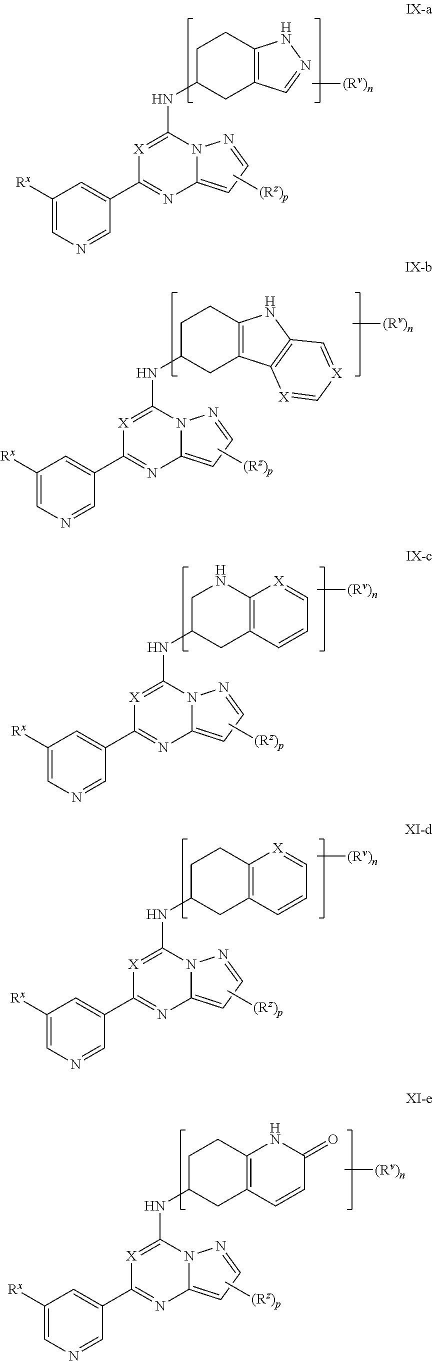 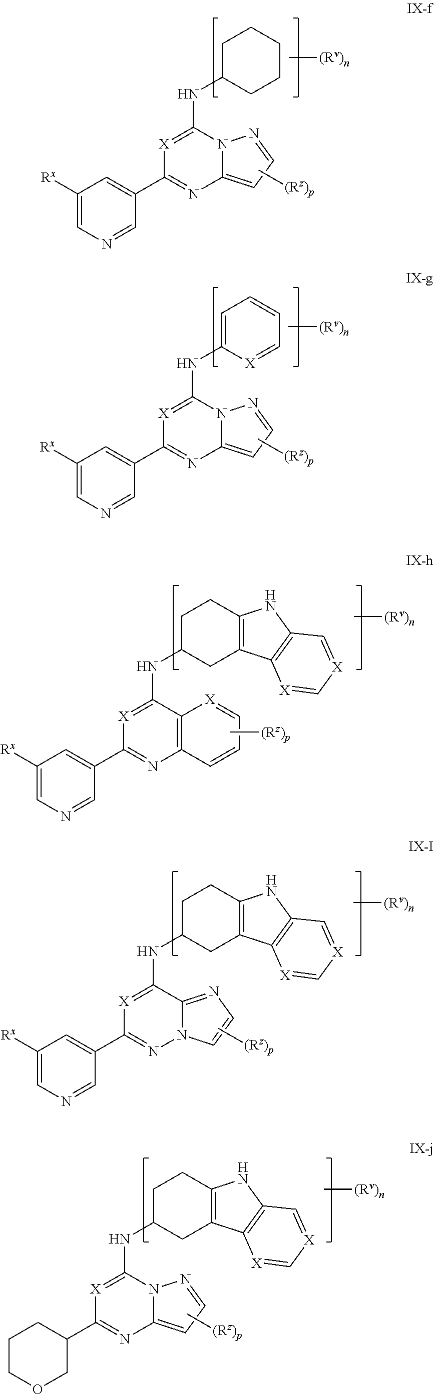 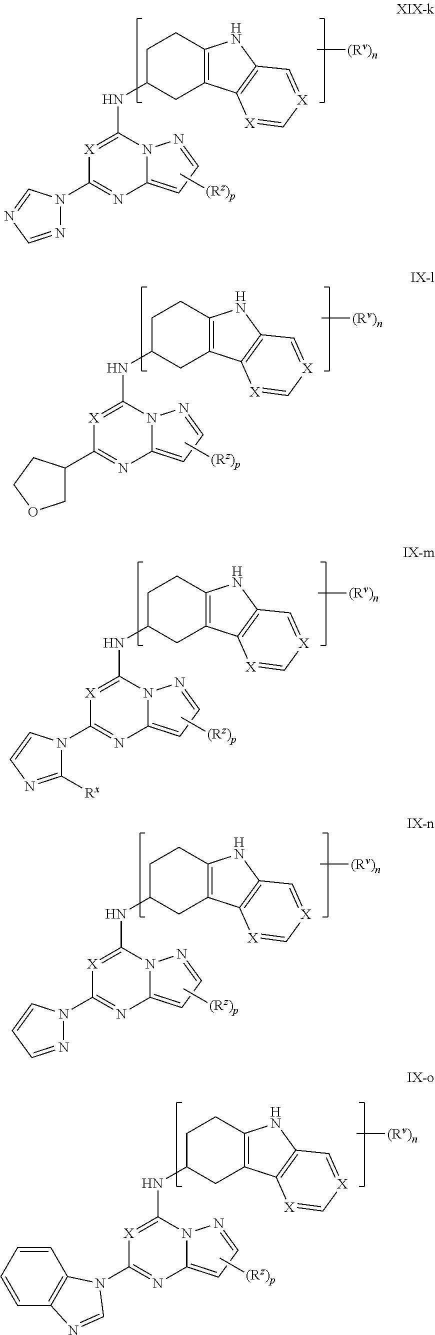 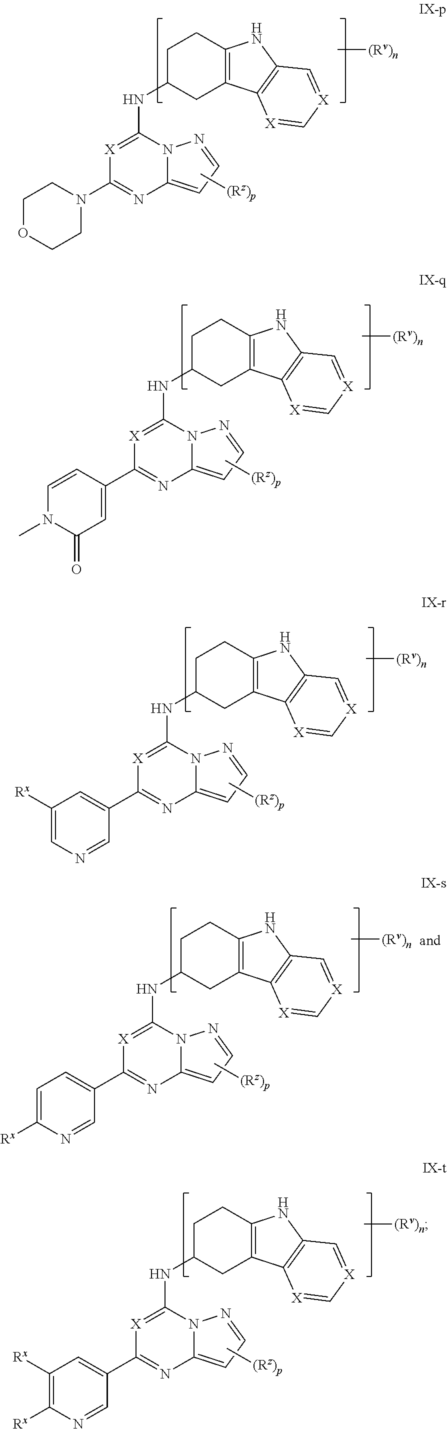
or a pharmaceutically acceptable salt thereof wherein X is N or CH and Z is CH₂, NH or O.

11. The compound according to claim 1 wherein the compound is selected from any of formulae X-a, X-b, X-c, X-d, X-e, X-f, X-g, X-h, X-i, X-j, X-k, X-l, X-m, X-n, X-o, X-p, X-q, X-r, X-s, X-t and X-u: 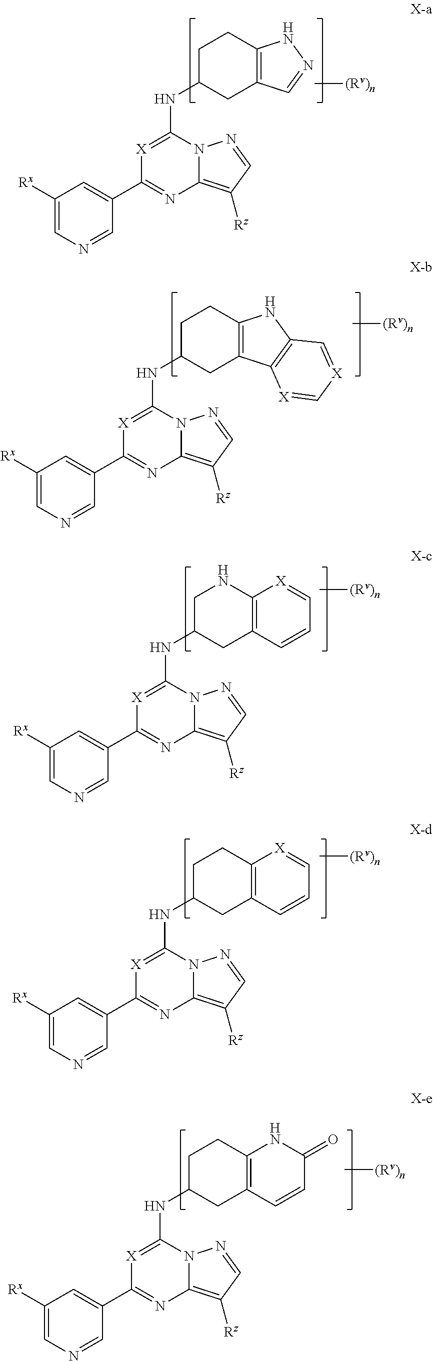 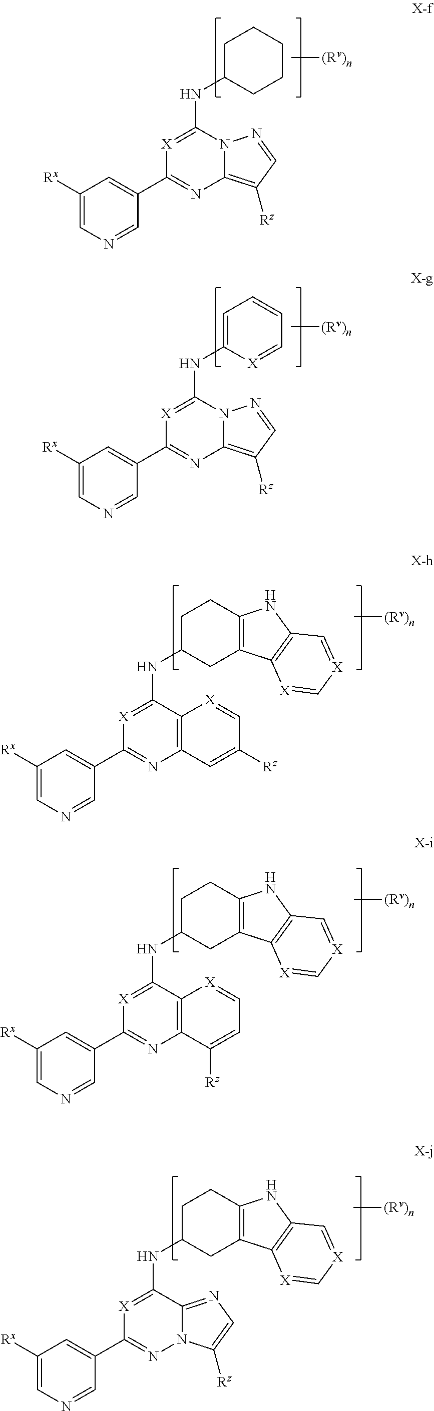 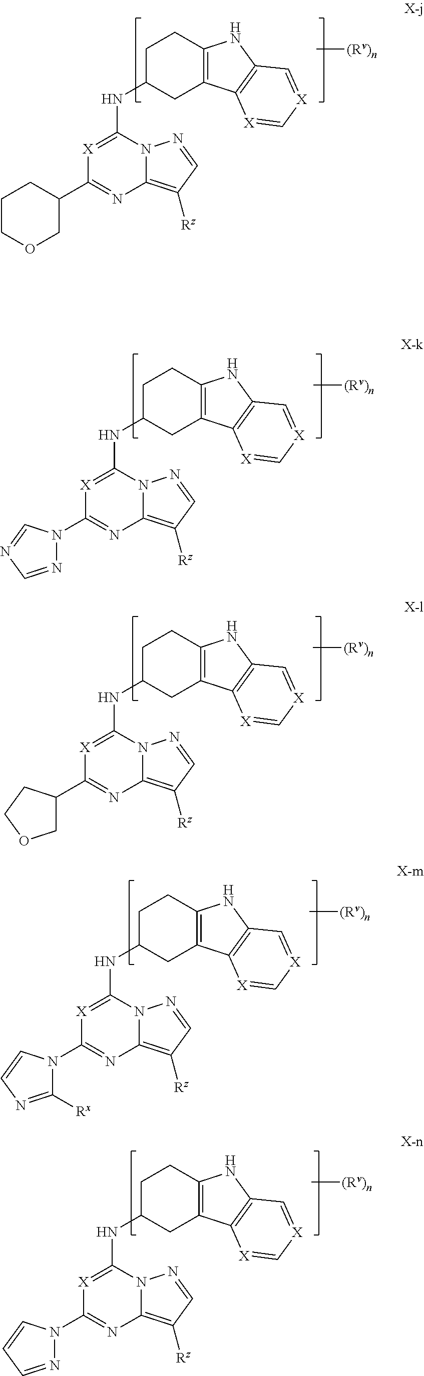 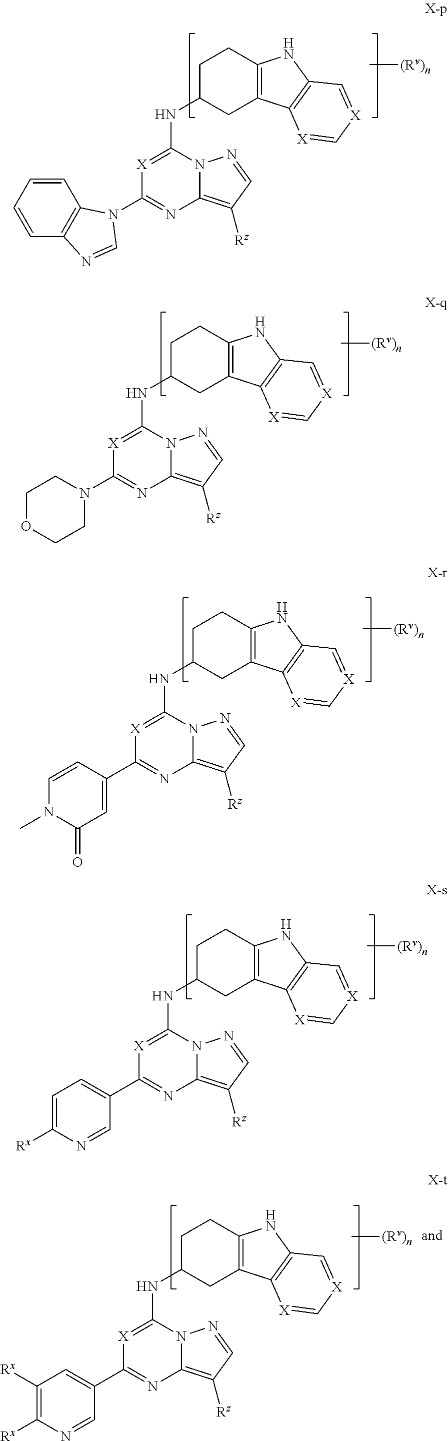 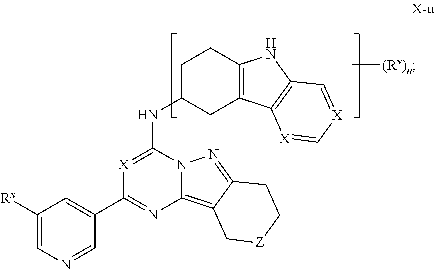
or a pharmaceutically acceptable salt thereof wherein X is N or CH and Z is CH₂, NH or O.

12. The compound according to claim 1 wherein the compound is selected from any of formulae XI-a, XI-b, XI-c and XI-d: 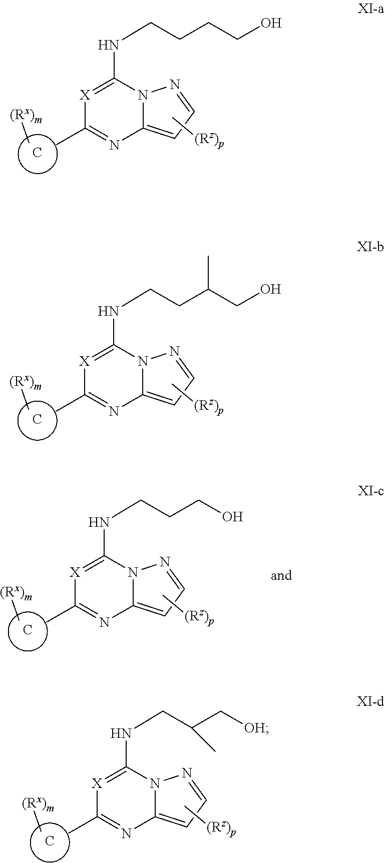
or a pharmaceutically acceptable salt thereof wherein X is N or CH.

13. The compound according to claim 1 wherein the compound is selected from those depicted in Table 1, or a pharmaceutically acceptable salt thereof.

14. A composition comprising the compound according to claim 1, or a pharmaceutically acceptable salt thereof, and a pharmaceutically acceptable carrier, adjuvant, or vehicle.

15. A method of inhibiting AHR in a patient in need thereof, comprising administering to said patient the compound according to claim 1, or a pharmaceutically acceptable salt thereof.

16. A method of inhibiting AHR in a biological sample, comprising contacting the biological sample with the compound according to claim 1, or a pharmaceutically acceptable salt thereof.

17. A method for treating an AHR-mediated disorder in a patient in need thereof, comprising administering to said patient the compound according to claim 1, or a pharmaceutically acceptable salt thereof.

18. The method according to claim 17, wherein the AHR-mediated disorder is a cancer.

19. The method according to claim 17, wherein the AHR-mediated disorder is an inflammatory disorder.

20-22. (canceled)
